(19) United States
(12) Patent Application Publication
(10) Pub. No.: US 2021/0174902 A1
(43) Pub. Date: Jun. 10, 2021

(54) RECOMBINASE DISCOVERY

(71) Applicant: Homodeus, Inc., Guilford, CT (US)

(72) Inventors: Harry Kemble, Paris (FR); Spencer Glantz, West Hartford, CT (US); Jonathan M. Rothberg, Guilford, CT (US)

(73) Assignee: Homodeus, Inc., Guilford, CT (US)

(21) Appl. No.: 17/117,921

(22) Filed: Dec. 10, 2020

Related U.S. Application Data
(60) Provisional application No. 62/946,196, filed on Dec. 10, 2019.

(51) Int. Cl. G16B 30/10; G16B 40/00

(52) U.S. Cl. CPC G16B 30/10; G16B 40/00

(57) ABSTRACT

The present disclosure provides methods, compositions, kits, and systems for identifying recombinases and cognate site-specific recombinase recognition sites as well as method for using the identified recombinase/recognition site pairs.

### RELATED APPLICATION

This application claims the benefit under 35 U.S.C. § 119(e) of U.S. provisional application No. 62/946,196, filed Dec. 10, 2019, which is incorporated by reference herein in its entirety.

### BACKGROUND

Site-specific recombinases are enzymes that catalyze precise DNA rearrangements, or recombination events, at specific DNA target site pairs (e.g., 30-150 nucleotides long each site). Each individual natural recombinase has evolved to act with some degree of specificity at its own unique recognition sites and not at other “off-target” DNA sites. DNA recombination events involve DNA breakage, strand exchange between homologous segments, and rejoining of the DNA. Site-specific recombinases can vastly differ in their overall amino acid composition, however, recombinases have individual sub-regions (domains), that are highly conserved across recombinase family members. To find new putative recombinases, one can simply search candidate genomic sequences for the presence of those conserved domains.

### SUMMARY

Provided herein, in some aspects, are methods that may be used to (i) identify genes that encode site-specific recombinases and (ii) predict the cognate recognition site pairs within target genomes that the recombinases recognize and recombine.

Some aspects of the present disclosure provide methods (e.g., computer implemented methods) comprising mining from a protein database (e.g., Conserved Domain Database (CDD)) putative recombinase sequences based on conserved recombinase domain architecture, linking the putative recombinase sequences to prokaryotic genomic sequences containing their corresponding coding sequences, scanning those genomic sequences to identify prophage sequences (using e.g., PHAST or PHASTER) containing the coding sequences, aligning those prophage sequences and their boundary-flanking sequences with homologous genomic sequences from the same genus to produce sequence alignments (e.g., using MegaBLAST), and automatically solving for putative cognate recombinase recognition sites by detecting overlapping sequences in the sequence alignments.

Other aspects of the present disclosure provide a computer readable medium on which is stored a computer program which, when implemented by a computer processor, causes the processor to mine from a protein database putative recombinase sequences based on conserved recombinase domain architecture or other measure of homology to known recombinases, link the putative recombinase sequences to prokaryotic genomic sequences containing their corresponding coding sequences, scan those genomic sequences to identify prophage sequences containing the coding sequences, align the prophage sequences and their boundary-flanking sequences with homologous genomic sequences from the same genus to produce sequence alignments, and automatically solve for putative cognate recombinase recognition sites by detecting overlapping sequences in the sequence alignments.

In some embodiments, the mining is based on a precisely ordered recombinase domain superfamily architecture.

In some embodiments, the linking includes accessing a database (e.g., Entrez Nucleotide database) that comprises annotated records.

In some embodiments, the linking includes automatically removing uninformative nucleotide sequences from the genomic coding sequences.

In some embodiments, the genomic coding sequences includes at least 2, at least 5, at least 10, at least 25, at least 50, or at least 100 annotated genomic coding sequences.

In some embodiments, the boundary-flanking sequences have a length of at least 20 kilobases (kb). For example, the boundary-flanking sequences may have a length of 20, 25, 30, 35, 40, 45, or 50 kb.

In some embodiments, the automatically solving includes defining multiple putative cognate recombinase recognition sites for a single recombinase.

In some embodiments, the automatically solving includes implementation of an algorithm that includes a measure of confidence in each predicted recombinase recognition site set, optionally in the form of ambiguity scores.

In some embodiments, the method is automated.

In some embodiments, the methods further comprise continuously updating the solved recombinase list as the protein database is updated.

In some embodiments, the methods further comprise verifying that all putative cognate recombinase recognition sites solved flank a sequence encoding at least one of the putative recombinase sequences.

In some embodiments, the putative recombinase sequences comprise tyrosine and/or serine recombinase sequences. In some embodiments, the serine recombinase sequences comprise resolvase and/or integrase sequences.

In some embodiments, the recombinases are thermostable. In some embodiments, the recombinases amino acid sequences contain one or more sub-sequences (e.g. nuclear localization signals) that collectively result in the transportation of the folded protein to a eukaryotic cell nucleus.

### BRIEF DESCRIPTION OF THE DRAWINGS

FIG. 1 is a flow diagram of the steps of an illustrative process for discovering recombinases and cognate recognition site pairs.

FIG. 2 is a block diagram of an illustrative implementation of a computer system for discovering recombinases and cognate recognition site pairs.

FIG. 3 is a schematic showing clustering of protein sequences by their homology to the cluster “centroid,” where all proteins in a given cluster share more than some threshold (e.g., 30%) degree of homology to the centroid, and are closer in homology space to their assigned cluster centroid than to any other cluster centroid.

FIG. 4 is a schematic showing recombinases cluster together in families according to their shared sequence homology. Clusters are defined in this figure as recombinases that give BLAST alignment e-values of <10E-10. Recombinases disclosed herein that have newly discovered recognition sites are light gray colored, and recombinases with previously published DNA target sites are medium gray colored.

FIG. 5 is a schematic comparing recombinase targets not yet present (left) and already present (right) at a desired recombination site.

### DETAILED DESCRIPTION

Making specific changes to nucleic acids in vitro, in cells, and in multicellular living organisms has been a major focus of the biotechnology community for decades. Precision DNA editing is important to the research community, which seeks to understand the role that the genome plays in cellular and organismal biology across the many kingdoms of life. Genome editing is also relevant to healthcare because it can serve as the basis for many therapeutic strategies. For example, gene editing tools may be used, among many other applications, to reprogram immune cells to seek out and eliminate cancer cells, make specific edits to patients' genomes to correct for disease-causing mutations, and/or engineer bacteriophage viruses such that they seek out and eliminate bacterial infections. Further, genome editing is important for the biotechnology industry as a whole. The agricultural industry has made genetically-engineered crops designed to better withstand harsh environmental conditions, such as drought or the presence of pathogens, and the genomes of domesticated animals have been modified to facilitate safe food production.

New site-specific recombinases that recombine DNA at previously unknown target (recognition) sites are useful as each one can unlock the power to make precise DNA edits at new genomic locations and enable at least the aforementioned applications. Unlike any of the other genome engineering enzymes commercially available today, including transposases and nucleases, site-specific recombinases can perform precision integration, excision, inversion, translocation, and cassette exchange with minimal off-targeting. In aggregate, having a large collection of recombinases and cognate recognition site pairs is also useful for enhancing our understanding of recombinase structure/function, which will, in turn, enable the design of new, engineered recombinases that edit DNA with high efficiency at target sites never before recombined in nature.

Aspects of the present disclosure uniquely combine two advantageous approaches for predicting the DNA recognition sites for a putative site-specific recombinase: in vitro assays used to quantify the physical interaction between a recombinase and a library of potential candidate DNA recognition sites and in silico methods used to identify genomic evidence of recombination by a particular recombinase at a particular DNA site. Unlike current methods, the methods of the present disclosure, in some embodiments, (i) include algorithmic advancements that improve the identification of new recombinases and cognate recognition site pairs, and/or (ii) are fully automated, thus providing consistent, predictable, fast and high-throughput performance, and/or (iii) include quality control steps for improved accuracy, and/or (iv) continuously access and scan public databases to identify new recombinases and cognate recognition site pairs as new sequencing data is deposited.

The in vitro methods depend on the availability of purified recombinase protein, and thus, have been low-throughput to date with respect to the numbers of unique recombinase: recognition site pairs that can be solved. Furthermore, in vitro assays designed to identify potential recognition sites among unbiased (all possible) DNA target (recognition) sites only consider recombinase:DNA binding and cannot make predictions regarding which sites will permit actual recombination. An in vitro method that does consider DNA recombination at a library of candidate sites requires the use of a biased DNA recognition site library that is based upon an excellent starting prediction as to the actual recognition site, and thus could not be used in cases where the recognition site must be predicted ab initio.

In silico methods are available for the prediction of recognition site pairs for the Cre-like subtype of the tyrosine recombinase family and the phage large serine integrase subtype of the serine recombinase family. Recognition site pair prediction for the latter is enabled by the known biology of phage large serine integrases: during the natural course of bacterial infection by a temperate bacteriophage, recombinase genes in the phage genome may be expressed. Phage-produced recombinase enzyme can then facilitate the insertion of the phage genome into the host bacterial genome at a specific bacterial DNA site. Therefore, sequencing data that reveals the presence of a prophage integrated into a bacterial genome contains evidence as to the DNA targets at which that recombination event occurred.

Large serine integrases, a particular type of serine recombinases, perform recombination between four (4) DNA target sites (attL, attR, attB and attP) with no known motif or bias, and so their discovery is all the more difficult. If a recombinase gene can be identified within an integrated prophage, and the sequence of the prophage in the context of its integration into the host bacterial genome is known, and the sequence of a similar host genome in the absence of prophage integration is known, the original DNA target sites (also known as “substrates”) can be predicted and matched with the site-specific recombinase that performed the integration at that precise genomic location.

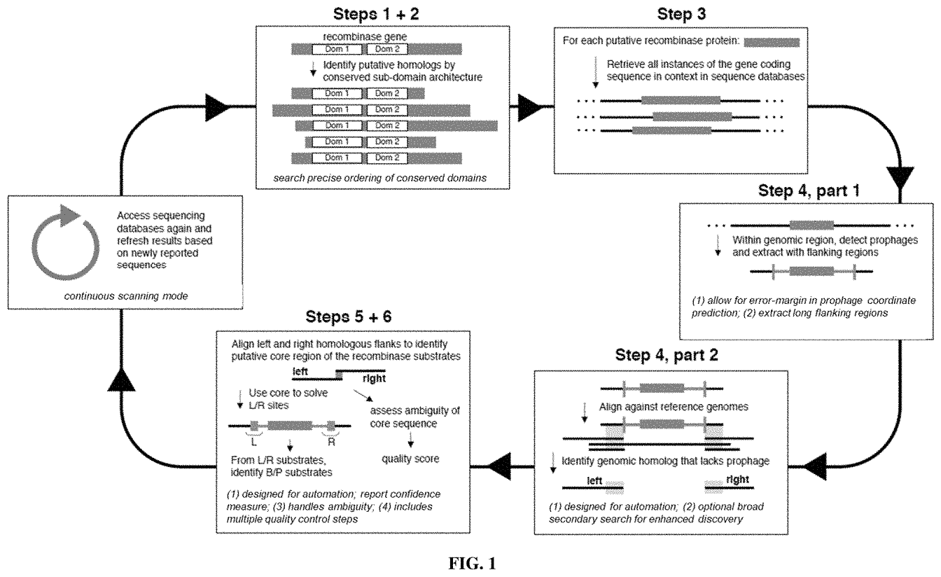

Aspects of the present disclosure comprise (1) mining from a protein database putative recombinase sequences based on conserved recombinase domain architecture, (2) linking the putative recombinase sequences to prokaryotic genomic sequences containing their corresponding coding sequences, (3) scanning those genomic sequences to identify prophage sequences containing the coding sequences, (4) aligning the prophage sequences and their boundary-flanking sequences with homologous genomic sequences from the same genus to produce sequence alignments, and/or (5) solving (e.g., automatically solving) for putative cognate recombinase recognition sites by detecting overlapping sequences in the sequence alignments. A flow chart of an exemplary method of the present disclosure is provided in FIG. 1. At least some of these steps may be implemented in software which can be carried out by a computing device. Thus, provided herein, in some embodiments, is a dynamic pipeline that, as sequencing databases grow in volume, continuously identifies recombinase genes and solves their cognate recognition sites (their associated DNA target sites) and improves the prediction quality for ambiguous target sites. In contrast to executing the method once at single point in time, a continuously operating pipeline results in increased recombinase and recombinase target site identification by constantly taking advantage of newly deposited sequences in sequencing databases.

Mining Protein Database(s)

In some embodiments, the methods comprise mining (e.g., automatically mining) from a protein database putative recombinase sequences based on conserved recombinase domain architecture. A set of precisely ordered conserved domain superfamily architectures characteristic of several known recombinase members may be defined, for example, by performing a conserved domain database search of the amino acid sequences of the known recombinase members. It should be understood that while described with respect to particular databases, the conserved domain database search is not limited to said particular databases. In some embodiments, the conserved domain database search is performed using any now known or later developed databases, each of which are contemplated to be within the scope of the present disclosure. Use, in some embodiments, of such a precisely ordered conserved domain architecture search to identify new recombinase genes (as opposed to a non-ordered conserved domain search) increases the probability that the identified putative recombinase sequences represent valid, functional recombinases. This in turn increases algorithmic speed by avoiding recognition site searches for low-quality, non-valid recombinases.

A protein (e.g., recombinase) domain is a conserved subsequence of a protein that can fold, function, and exist at least somewhat independently of the rest of the protein chain or structure. A domain architecture is the sequential order of conserved domains (functional units) in a protein sequence. Protein domains classified by CATH (class, architecture, topology, homology), for example, include Class 1 alpha-helices and Class 2 beta-sheets, e.g., α Horseshoes, α solenoides, aa barrels, 5-bladed β propellers, 3-layer (βββ) sandwiches, α/β super-rolls, 3-layer (βαβ) sandwiches, and α/β prisms (see, e.g., Nucleic Acids Res. 2009 January; 37 (Database issue): D310-D314). In some embodiments, a conserved recombinase domain is selected from members of the National Center for Biotechnology Information (NCBI) Conserved Domain (CD) Ser_Recombinase Superfamily (c102788) (comprising e.g., the NCBI CD Ser_Recombinase domain (cd00338), the SMART Resolvase domain (smart00857) and the Pfam Resolvase domain (pfam00239)), members of the NCBI CD PinE Superfamily (c134383) (comprising, e.g., the COG Site-specific recombinases, DNA invertase Pin homologs domain COG1961), members of the NCBI CD Recombinase Superfamily (c106512) (comprising e.g., the Pfam Recombinase domain (pfam07508)), members of the NCBI CD Zn_ribbon_recom Superfamily (c119592) (comprising e.g., the Pfam Zn_ribbon_recom domain (pfam13408), the Pfam Ogr_Delta domain (pfam04606) and the NCBI Protein Clusters domain PRK09678), members of the NCBI CD DNA_BRE_C Superfamily (c100213) (comprising e.g., the NCBI Protein Clusters domains PHA02731, PRK09870 and PRK09871, the Pfam Integrase_1 domain (pfam12835), the Pfam Phage_integrase domain (pfam00589), the Pfam Phage_integr_3 domain (pfam16795), and the Pfam Topoisom_I domain (pfam01028)), members of the NCBI CD XerC Superfamily (c128330) (comprising, e.g., the COG XerC domains COG0582 and COG4973, the COG XerD domain COG4974, the NCBI Protein Clusters domains PRK15417, PHA02601, PRK00236, PRK00283, PRK01287, PRK02436 and PRK05084, the TIGRFAMs recomb_XerC domain (TIGR02224) and the TIGRFAMs recomb_XerD domain (TIGR02225)), members of the NCBI CD Phage_int_SAM_1 Superfamily (c112235) (comprising, e.g., the Pfam Phage_int_SAM_1 domain (pfam02899) and the Pfam Phage_int_SAM_4 domain (pfam13495)), and members of the NCBI CD Arm-DNA-bind_l Superfamily (c107565) (comprising, e.g., the Pfam Arm-DNA-bind_l domain (pfam09003)) (see, e.g., Smith M C, Thorpe H M. Mol Microbiol. 2002; 44:299-307; Li W, et al. Science. 2005; 309:1210-1215; and Rutheford K, et al. Nucleic Acids Res. 2013; 41:8341-8356). In some embodiments, a conserved recombinase domain superfamily architecture is defined as an N-terminal NCBI CD Ser_Recombinase Superfamily (c102788), followed by NCBI CD Recombinase Superfamily (c106512), followed by any conserved domain(s) or no conserved domain, or by a sequence containing a coiled-coil motif.

The protein database used to mine putative recombinase sequences, in some embodiments, is the Conserved Domain Database (CDD) (ncbi nlm nih gov/Structure/cdd/cdd_help.shtml). The CDD can be used in some embodiments to identify protein similarities across significant evolutionary distances using sensitive domain profiles rather than direct sequence similarity. In some embodiments, given one or more protein query sequences, such as recombinase sequences, CD-Search (ncbi nlm nih gov/Structure/cdd/cdd_help.shtml#CDSearch_help_contents), Batch CD-search (ncbi nlm nih gov/Structure/cdd/cdd_help.shtml#BatchCDSearch_help_contents) or CDART (ncbi nlm nih gov/Structure/lexington/docs/cdart_about.html) can be used to reveal the conserved domains that make up a protein, as identified by RPS-BLAST. In some embodiments, CDART can be further be used to list proteins with a similar conserved domain architecture. In some embodiments, a query is submitted as a (a) protein sequence (in the form of a sequence identifier or as sequence data), (b) set of conserved domains (in the form of superfamily cluster IDs, conserved domain accession numbers, or PSSM IDs), or as (c) multiple queries.

In other embodiments, a protein sequence record is retrieved from another protein database, such as the Entrez Protein database, which is a collection of sequences from several sources, including translations from annotated coding regions in GenBank, RefSeq and Third Party Annotation (TPA), as well as records from SwissProt, the Protein Information Resource (PIR), Programmed Ribosomal Frameshift Database (PRFdb), and the Protein Data Bank (PDB) (www.ncbi.nlm nih.gov/protein).

Linking Recombinases to Coding Sequences

In some embodiments, the methods comprise linking (e.g., automatically linking) the putative recombinase sequences to corresponding genomic coding sequences. For each putative recombinase protein, more than one gene, and in some embodiments, all genes encoding the putative recombinase are identified (e.g., from sequenced genomes in the NCBI Entrez Nucleotide database). In some embodiments, at least 5, at least 10, at least 25, at least 50, at least 100, or at least 1000 genes encoding the putative recombinase are identified. Retrieving many or even all annotated coding sequences for each putative site-specific recombinase gene (as opposed to just a single coding sequence) increases the probability of detecting one or more instances where sufficient genetic information is available for the recombinase's recognition site to be solved. Multiple examples also open up the possibility of solving several sets of DNA target sites for a single putative integrase encoded from different genetic contexts, providing biological replicates. This additional information improves the quality of the recognition site prediction by suggesting the specificity of a recombinase for its recognition sites.

The linking step(s), in some embodiments, includes accessing a database that comprises annotated records of genomes assembled from long-read nucleotide sequences (e.g., technology from PacBio or Nanopore), short-read nucleotide sequences (e.g., Illumina next-generation sequencing reads), or a combination of long- and short-read nucleotide sequences, or directly annotated records of long-read nucleotide sequences. The database may be, for example, the Identical Protein Groups database, which is a resource that contains a single entry for each protein translation found in several sources at NCBI, including annotated coding regions in GenBank and RefSeq, as well as records from SwissProt and PDB.

In some embodiments, an automated filtering process is used to filter unusable putative recombinase coding sequences (e.g., engineered variants). For example, genomic sequences carrying already known integrase genes, or those derived from plasmids or non-integrated phages may be removed.

Scanning Prophage Database(s)

In some embodiments, the methods comprise scanning (e.g., automatically scanning) the prokaryotic genomic sequences containing the putative integrase coding sequences for signals of prophages, to identify and locate prophage sequences. In some embodiments, prophage sequences are identified using a prophage-detection program (web-based or locally executable) selected from PHASTER, PHAST, Prophage Hunter, Prophinder, and PhiSpy (see, e.g., Arndt D et al. Nucleic Acids Res. 2016 Jul. 8; 44(W1):W16-21; Zhou Y et al. Nucleic Acids Res. 2011 July; 39(Web Server issue):W347-52; Song W et al. Nucleic Acids Research, 2019; 47(W1): W74-W80; Lima-Mendez G et al. Bioinformatics. 2008 Mar. 15; 24(6):863-5; Akhter S et al. Nucleic Acids Res. 2012 September; 40(16): e126). In some embodiments, default program parameters are used. For locally-executable programs, FASTA files, for example, containing all the unique nucleotide sequences named in the filtered IPG record tables can be first downloaded to use as the input for the prophage-detection program, using, for example, the Entrez Utilities command, EFetch (with parameters: db=“nuccore”, id=[Nucleotide record accession.version], retype=“FASTA”).

For each putative prophage predicted to contain one or more of the putative recombinase coding sequences, the DNA sequence containing the putative prophage region and at least 10, at least 15, or at least 20 kilobases (kb) upstream and downstream of the putative prophage region is extracted and searched for alignments against all the non-redundant homologous genomes belonging to the same genus as the putative prophage host. In some embodiments, for each putative prophage predicted to contain one or more of the putative recombinase coding sequences, the DNA sequence containing the putative prophage region and approximately 20 kb upstream and downstream of the putative prophage region is extracted. In some embodiments, this alignment is done using the NCBI Megablast program, optionally with default parameters. The process of identifying genus-specific reference genomes may be automated, for example, enabling a more comprehensive search in less time. In some embodiments, an error-margin is allowed in the initial prediction of prophage coordinates, as opposed to a more stringent coordinate setting. This error-margin increases the probability that recombinase target sites can be solved by avoiding premature discounting of recombinase coding sequences that do not lie within the originally predicted prophage coordinates but may later be discovered to indeed lie within the precisely solved prophage coordinates. Further, by increasing the error-margin allowance in identification of prophage-flanking regions used for reference genome searching, for example, extracting at least 20 kb of sequence flanking the prophage region for alignment against reference sequences increases the chance of correctly finding the prophage boundaries and thus improves the hit rate of target site solving (compared to allowing smaller error-margins and extracting, e.g., ˜10 kb flanking sequences).

In the event that a genus-specific reference genome search fails, a broader reference genome set (all whole genome prokaryotic sequences in the sequencing database) may be searched (rather than simply marking the attempt a failure after the primary, narrower search). This secondary, broad reference genome search increases the probability that recombinase substrates can be identified even for recombinase genes embedded in prophages integrated into host genomes that do not have a readily available identifiable reference genome already annotated at the genus level.

Aligning Prophage Sequences

In some embodiments, the methods comprise aligning (e.g., automatically aligning) the prophage sequences and their boundary-flanking sequences with homologous genomic sequences from the same genus to produce sequence alignments. If a homologous genomic sequence lacking the integrated prophage is present in the alignment reference database, the precise prophage boundaries in the query sequence may be detected as a small (e.g., 2-18 base pairs (bp)) overlap between multiple alignment ranges in a reference genomic sequence, corresponding to the left and right prophage-flanking regions. In some embodiments, the overlap of the phage boundary alignment ranges is 2-50 base pairs (bp). For example, the overlap of the phage boundary alignment ranges may be 2-40, 2-30, 2-20, 5-40, 5-30, 5-20, 10-40, 10-30, or 10-20 bp. Putative recombinase recognition sites (e.g., attL, attR, attB and attP) may be inferred from the, e.g., 59-66 bp, sequences centered on the core sequence defined by this overlap. In some embodiments, putative recombinase recognition sites are inferred from 30-100 bp sequences centered on the core sequence. For example, putative recombinase recognition sites may be inferred from 30-90, 30-80, 30-70, 30-60, 40-90, 40-80, 40-70, 40-60, 50-90, 50-80, 50-70, or 50-60 bp sequences centered on the core sequence.

In some embodiments, a strategy is applied to extract useful information from (relatively common) cases where the sequences of a “left overlap” and “right overlap” are non-identical. This increases the probability of obtaining target site information for a given recombinase (see, e.g., FIG. 1, Steps 4-6).

Further, instead of basing att site inferences on just a single alignment, in some embodiments, multiple or all pairs of “left overlap” and “right overlap” detected from the alignment output can be considered to potentially define a list of att core sequences associated with a given prophage. This increases the chances of defining an unambiguous core sequence for a given prophage's att sites, as well as provides other information relating to the confidence in the inferred att sites of a given prophage.

Solving Recombinase Recognition Site(s)

In some embodiments, the methods comprise solving (e.g., automatically solving) for putative cognate recombinase recognition sites by detecting overlapping sequences in the sequence alignments. In some embodiments, this step involves fully automated application of a rapid and sensitive algorithm for solving recombinase target sites from the boundary regions of host genome-integrated prophages using alignments.

The algorithm may also assess the number of total integrase genes harbored within a given prophage, which provides a measure of confidence as to the likelihood of any particular integrase acting on the associated prophage boundary substrates, increasing the accuracy of the overall algorithm. The algorithm used for solving putative cognate recombinase recognition sites includes, in some embodiments, a measure of confidence in each predicted recombinase recognition site set, in the form of ambiguity scores, which increase the quality of the prediction by providing an assessment of its validity.

In some embodiments, a verification step is included to ensure that a putative recombinase is only ascribed to a particular target pair if it has a coding sequence located within the precisely solved prophage boundaries (not just the imprecise original initial estimate of the prophage boundaries computed earlier in the pipeline). This verification step increases the accuracy of recombinase and cognate target recognition site prediction by eliminating unlikely pairings.

Recombinases and Recombination Recognition Sequences

Recombinases are enzymes that mediate site-specific recombination (site-specific recombinases) by binding to nucleic acids via conserved DNA recognition sites (e.g., between 30 and 100 base pairs (bp)) and mediating at least one of the following forms of DNA rearrangement: integration, excision/resolution, inversion, translocation, and/or cassette exchange.

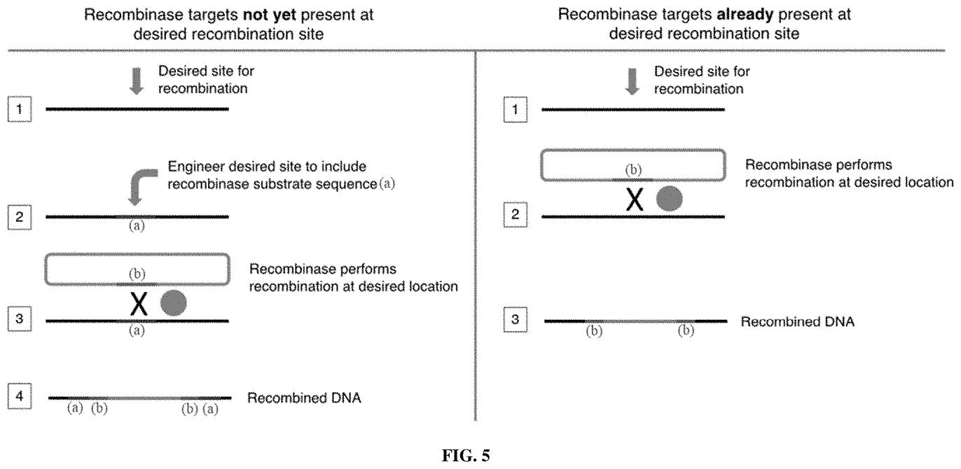

A site-specific recombinase may be used outside of its natural context in at least two ways: (1) one or more recombinase recognition sites are first engineered into one or more target nucleic acids and then a recombinase is used to perform the desired rearrangement, or (2) a recombinase is used to recombine one or more nucleic acids at their recognition site(s), which were already present in the target nucleic acid (see, e.g., FIG. 5). The latter approach is more elegant, involves time and cost savings, and thus is preferable, in some instances. To the extent that new site-specific recombinases and more potential DNA substrates are identified, each increases the likelihood that one can perform recombination at a target site of interest without having to first introduce the DNA substrate sequence.

Recombinases can be classified into two distinct families: serine recombinases (e.g., resolvases and invertases) and tyrosine recombinases (e.g., integrases), based on distinct biochemical properties. Serine recombinases and tyrosine recombinases are further divided into bidirectional recombinases and unidirectional recombinases. Examples of bidirectional serine recombinases include, without limitation, β-six, CinH, ParA and γδ; and examples of unidirectional serine recombinases include, without limitation, Bxb1, ϕC31, TP901, TG1, φBT1, R4, φRV1, φFC1, MR11, A118, U153 and gp29. Examples of bidirectional tyrosine recombinases include, without limitation, Cre, FLP, and R; and unidirectional tyrosine recombinases include, without limitation, Lambda, HK101, HK022 and pSAM2. The serine and tyrosine recombinase names stem from the conserved nucleophilic amino acid residue that the recombinase uses to attack the DNA and which becomes covalently linked to the DNA during strand exchange. Recombinases have been used for numerous standard biological applications, including the creation of gene knockouts and the solving of sorting problems.

The outcome of recombination depends, in part, on the location and orientation of two short DNA sequences that are to be recombined (typically less than 60 bp long). Recombinases bind to these target sequences, which are specific to each recombinase, and are herein referred to as recombinase recognition sites. Recombinases may recombine two identical, repeated recognition sites or two dissimilar, non-identical recognition sites. Thus, as used herein, a recombinase is specific for a pair of recombinase recognition sites when the recombinase can mediate intramolecular inversion, intramolecular excision or intramolecular circularization between two recognition DNA sequences or when the recombinase can mediate intermolecular translocation, or intermolecular integration for two DNA sequences, each containing to one of the two DNA recognition sequences. As used herein, a recombinase may also be said to be specific for a recombinase recognition site when two simultaneous intermolecular translocation reactions are used to drive intermolecular cassette exchange between two recognition DNA sequences on two different DNA molecules. As used herein, a recombinase may also be said to recognize its cognate recombinase recognition sites, which flank or are adjacent to an intervening piece of DNA (e.g., a gene of interest or other genetic element). A piece of DNA is said to be flanked by a pair of recombinase recognition sites when the piece of DNA is located between and immediately adjacent to the sites.

A subset of the site-specific recombinases provided herein have DNA target sites that are exact or near matches to sequences in natural prokaryotic genomes. Thus, these recombinases can be used directly to engineer the genome of the prokaryotic organism with no prior engineering work. This is particularly valuable, for example, for the introduction of new DNA into a genome (e.g., for research, therapeutic or industrial purposes) and especially for organisms that are otherwise challenging to manipulate with current genetic engineering approaches, such as gram-positive bacteria. Co-transformation of an engineered nucleic acid vector that results in the expression of a recombinase and a donor DNA vector that contains one recombinase recognition site could be used to integrate the donor DNA specifically into the natural bacterial genome at the precise location that naturally contains the second recombinase recognition sequence.

Having more and new site-specific recombinases also increases the probability of identifying a set of multiple, “orthogonal” site-specific recombinases that act on distinct enough target pair sites that there is no recombination cross-talk. Sets of orthogonal site-specific recombinases are highly useful for engineering genetic “logic circuits” where a logical output (e.g., gene expression, orientation of primer-binding sites, etc.) can be computed by the rearrangement of DNA segments located between unique pairs of recombinase target sites.

While many site-specific recombinases are known to exhibit recombination activity in vitro, their relative efficiencies differ with respect to recombination in cells or in an organism (in vivo). Site-specific recombinases that are thermostable, and/or contain nuclear localization signals (NLS), have been shown to perform with higher efficiency in vivo, and are therefore of high value, especially if they act on previously unknown target sequences.

Making specific changes to nucleic acids in vitro, in cells and in multicellular living organisms has been a major focus of the biotechnology community for decades. Precision DNA editing is incredibly important to the research community, which seeks to understand the role that the genome plays in cellular and organismal biology across the many kingdoms of life. Genome editing is also relevant to healthcare because it can serve as the basis for many therapeutic strategies. For example, gene editing tools may be used to re-program immune cells in order that they seek out and eliminate cancer cells; make specific edits to patients' genomes to correct for disease-causing mutations; and engineer bacteriophage viruses such that they seek out and eliminate bacterial infections, among many other applications. Lastly, genome editing is important for the biotechnology industry as a whole. The agricultural industry has made genetically-engineered crops designed to better withstand harsh environmental conditions, such as drought or the presence of pathogens, and the genomes of domesticated animals have been modified to facilitate safe food production, for example.

Inversion recombination happens between a pair of short recombinase target DNA sequences on the same molecule in “head-to-head” relative orientation. A DNA loop formation brings the two target sequences together at a point of strand-exchange. The end result of such an inversion recombination event is that the stretch of DNA between the target sites inverts (i.e., the stretch of DNA reverses orientation). In such reactions, the DNA is conserved with no net gain or loss of DNA or its bonds.

Conversely, excision recombination occurs between two short DNA target sequences on the same molecule that are oriented in the same direction. In this case, the intervening DNA is excised/removed as a DNA circle. Thus, excision recombination may be used to circularize an intervening DNA sequence that is flanked by DNA recognition sequences while simultaneously resulting in excision of the intervening DNA sequence from the parent DNA molecule, which may be linear or circular.

Translocation recombination occurs between two short DNA recognition sequences that are oriented in the same direction but are located on two distinct DNA molecules. In this case, the DNA sequence that is located downstream of the 3′ end of one of the recognition sequences is exchanged with the DNA located downstream of the 3′ end of the other corresponding recognition sequence on a second DNA molecule. Thus, translocation recombinase may be used to generate chimeric DNA molecules consisting of sub-sequences that originated from distinct parent DNA molecules.

Integrating recombination occurs between two short DNA recognition sequences that are oriented in the same direction, but are located on two distinct DNA molecules, and where at least one of the DNA molecules is circular. In this case, recombination results in the integration of the circular “donor” DNA in its entirety into the second DNA molecule, which may be circular or linear, at the recognition sequence site.

Intermolecular cassette exchange occurs between 4 short DNA recognition sequences that are all oriented in the same direction, but where 2 short recognition sequences flank an intervening DNA sequence on one molecule and the other 2 short recognition sequences flank an intervening DNA sequence on a second DNA molecule. The 4 short recognition sequences can consist of two identical pairs of recognition sites for a given site-specific recombinase or can consist of two distinct recognition site pairs, where one pairing is at the 5′ end of the intervening DNA sequence on both molecules and one pair is at the 3′ end of the intervening DNA sequence on both molecules. Simultaneous or serial translocation reactions result in the precise intermolecular exchange of the intervening DNA sequence between the two pairs of flanking recognition sequences. Thus, cassette exchange may be used to replace a particular stretch of DNA with new donor DNA without requiring the integration of the complete donor DNA molecule, as what occurs in integrating recombination.

Recombinases can also be classified as irreversible or reversible. An irreversible recombinase refers to a recombinase that can catalyze recombination between two complementary recombination sites, but cannot catalyze recombination between the hybrid sites that are formed by this recombination without the assistance of an additional factor. Thus, an irreversible recognition site is a recombinase recognition site that can serve as the first of two DNA recognition sequences for an irreversible recombinase and that is modified to a hybrid recognition site following recombination at that site. A complementary irreversible recognition site is a recombinase recognition site that can serve as the second of two DNA recognition sequences for an irreversible recombinase and that is modified to a hybrid recombination site following recombination at that site. For example, attB and attP, are the irreversible recombination sites for Bxb1 and phiC31 recombinases—attB is the complementary irreversible recombination site of attP, and vice versa. The attB/attP sites can be mutated to create orthogonal B/P pairs that only interact with each other but not the other mutants. This allows a single recombinase to control the excision or integration or inversion of multiple orthogonal B/P pairs.

The phiC31 (φC31) integrase, for example, catalyzes only the attB x attP reaction in the absence of an additional factor not found in eukaryotic cells. The recombinase cannot mediate recombination between the attL and attR hybrid recombination sites that are formed upon recombination between attB and attP. Because recombinases such as the phiC31 integrase cannot alone catalyze the reverse reaction, the phiC31 attB x attP recombination is stable.

Irreversible recombinases, and nucleic acids that encode the irreversible recombinases, are described in the art and can be obtained using routine methods. Examples of irreversible recombinases include, without limitation, phiC31 (φC31) recombinase, coliphage P4 recombinase, coliphage lambda integrase, Listeria A118 phage recombinase, and actinophage R4 Sre recombinase, HK101, HK022, pSAM2, Bxb1, TP901, TG1, φBT1, φRV1, φFC1, MR11, U153 and gp29.

Conversely, a reversible recombinase is a recombinase that can catalyze recombination between two complementary recombinase recognition sites and, without the assistance of an additional factor, can catalyze recombination between the sites that are formed by the initial recombination event, thereby reversing it. The product-sites generated by recombination are themselves substrates for subsequent recombination. Examples of reversible recombinase systems include, without limitation, the Cre-lox and the Flp-frt systems, R, β-six, CinH, ParA and γδ.

The recombinases provided herein are not meant to be exclusive examples of recombinases that can be used in embodiments of the present disclosure. The complexity of logic and memory systems of the present disclosure can be expanded by mining databases for new orthogonal recombinases or designing synthetic recombinases with defined DNA specificities. Other examples of recombinases that are useful are known to those of skill in the art, and any new recombinase that is discovered or generated is expected to be able to be used in the different embodiments of the present disclosure.

In some embodiments, the recombinase is serine or tyrosine integrase. Thus, in some embodiments, the recombinase is considered to be irreversible. In some embodiments, the recombinase is a serine or tyrosine invertase, resolvase or transposase. Thus, in some embodiments, the recombinase is considered to be reversible. Unidirectional recombinases bind to non-identical recognition sites and therefore mediate irreversible recombination. Examples of unidirectional recombinase recognition sites include attB, attP, attL, attR, pseudo attB, and pseudo attP. In some embodiments, the circuits described herein comprise unidirectional recombinases.

Examples of unidirectional recombinases include but are not limited to Bxb1, PhiC31, TP901, HK022, HP1, R4, Int1, Int2, Int3, Int4, Int5, Int6, Int7, Int8, Int9, Int10, Int11, Int12, Int13, Int14, Int15, Int16, Int17, Int18, Int19, Int20, Int21, Int22, Int23, Int24, Int25, Int26, Int27, Int28, Int29, Int30, Int31, Int32, Int33, and Int34. Further unidirectional recombinases may be identified using the methods disclosed in Yang et al., Nature Methods, October 2014; 11(12), pp. 1261-1266, herein incorporated by reference in its entirety.

Examples of bidirectional recombinases include, but are not limited to, Cre, FLP, R, IntA, Tn3 resolvase, Hin invertase and Gin invertase.

In some embodiments, a recombinase is a bacterial recombinase. Non-limiting examples of bacterial recombinases include FimE, FimB, FimA and HbiF. HbiF is a recombinase that reverses recombination sites that have been inverted by Fim recombinases. Bacterial recombinases can recognize inverted repeat sequences, termed inverted repeat right (IRR) and inverted repeat left (IRL).

Some aspects of the present disclosure provide engineered recombinases comprising an amino acid sequence having at least 70% identity to an amino acid sequence of any one of SEQ ID NOs: 1-395. For example, an engineered recombinase may comprise an amino acid sequence having at least 75%, at least 80%, at least 85%, at least 90%, at least 95%, at least 96%, at least 97%, at least 98%, at least 99%, or 100% identity to an amino acid sequence of any one of SEQ ID NOs: 1-395. In some embodiments, an engineered recombinase comprises an amino acid sequence having 70%-80%, 70%-90%, 70%-100%, 80%-90%, 80%400%, or 90%-100% identity to an amino acid sequence of any one of SEQ ID NOs: 1-395.

“Identity” refers to a relationship between the sequences of two or more polypeptides (e.g. recombinases) or polynucleotides (nucleic acids), as determined by comparing the sequences. Identity also refers to the degree of sequence relatedness between or among sequences as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”) Identity of related polypeptides or nucleic acids can be readily calculated by known methods. “Percent (%) identity” as it applies to polypeptide or polynucleotide sequences is defined as the percentage of residues (amino acid residues or nucleic acid residues) in the candidate amino acid or nucleic acid (nucleotide) sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. It is understood that identity depends on a calculation of percent identity but may differ in value due to gaps and penalties introduced in the calculation. Generally, a particular polynucleotide or polypeptide (e.g., recombinase) has at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al (1997), “Gapped BLAST and PSI-BLAST: a new generation of protein database search programs”, Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197). A general global alignment technique based on dynamic programming is the Needleman-Wunsch algorithm (Needleman, S. B. & Wunsch, C. D. (1970) “A general method applicable to the search for similarities in the amino acid sequences of two proteins.” J. Mol. Biol. 48:443-453). More recently a Fast Optimal Global Sequence Alignment Algorithm (FOGSAA) has been developed that purportedly produces global alignment of nucleotide and protein sequences faster than other optimal global alignment methods, including the Needleman-Wunsch algorithm.

Engineered Nucleic Acids

Aspects of the present disclosure provide engineered nucleic acids encoding a recombinase as described herein. In some embodiments, an engineered nucleic encodes a recombinase comprising an amino acid sequence having at least 70% identity to an amino acid sequence of any one of SEQ ID NOs: 1-395. For example, an engineered nucleic may encode a recombinase comprising an amino acid sequence having at least 75%, at least 80%, at least 85%, at least 90%, at least 95%, at least 96%, at least 97%, at least 98%, at least 99%, or 100% identity to an amino acid sequence of any one of SEQ ID NOs: 1-395. In some embodiments, an engineered nucleic encodes a recombinase comprising an amino acid sequence having 70%-80%, 70%-90%, 70%-100%, 80%-90%, 80%-100%, or 90%-100% identity to an amino acid sequence of any one of SEQ ID NOs: 1-395.

A nucleic acid is at least two nucleotides covalently linked together, and in some instances, may contain phosphodiester bonds (e.g., a phosphodiester “backbone”). An engineered nucleic acid is a nucleic acid that does not occur in nature. It should be understood, however, that while an engineered nucleic acid as a whole is not naturally-occurring, it may include nucleotide sequences that occur in nature. In some embodiments, an engineered nucleic acid comprises nucleotide sequences from different organisms (e.g., from different species). For example, in some embodiments, an engineered nucleic acid includes a murine nucleotide sequence, a bacterial nucleotide sequence, a human nucleotide sequence, and/or a viral nucleotide sequence. Engineered nucleic acids include recombinant nucleic acids and synthetic nucleic acids. A recombinant nucleic acid is a molecule that is constructed by joining nucleic acids (e.g., isolated nucleic acids, synthetic nucleic acids or a combination thereof) and, in some embodiments, can replicate in a living cell. A synthetic nucleic acid is a molecule that is amplified or chemically, or by other means, synthesized. A synthetic nucleic acid includes those that are chemically modified, or otherwise modified, but can base pair with naturally-occurring nucleic acid molecules. Recombinant and synthetic nucleic acids also include those molecules that result from the replication of either of the foregoing.

In some embodiments, a nucleic acid of the present disclosure is considered to be a nucleic acid analog, which may contain, at least in part, other backbones comprising, for example, phosphoramide, phosphorothioate, phosphorodithioate, O-methylphophoroamidite linkages and/or peptide nucleic acids. A nucleic acid may be single-stranded (ss) or double-stranded (ds), as specified, or may contain portions of both single-stranded and double-stranded sequence. In some embodiments, a nucleic acid may contain portions of triple-stranded sequence. A nucleic acid may be DNA, both genomic and/or cDNA, RNA or a hybrid, where the nucleic acid contains any combination of deoxyribonucleotides and ribonucleotides (e.g., artificial or natural), and any combination of bases, including uracil, adenine, thymine, cytosine, guanine, inosine, xanthine, hypoxanthine, isocytosine and isoguanine.

Engineered nucleic acids of the present disclosure may include one or more genetic elements. A genetic element is a particular nucleotide sequence that has a role in nucleic acid expression (e.g., promoter, enhancer, terminator) or encodes a discrete product of an engineered nucleic acid.

Engineered nucleic acids of the present disclosure may be produced using standard molecular biology methods (see, e.g., Green and Sambrook, Molecular Cloning, A Laboratory Manual, 2012, Cold Spring Harbor Press).

In some embodiments, engineered nucleic acids are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the 3′ extension activity of a DNA polymerase and DNA ligase activity. The 5′ exonuclease activity chews back the 5′ end sequences and exposes the complementary sequence for annealing. The polymerase activity then fills in the gaps on the annealed regions. A DNA ligase then seals the nick and covalently links the DNA fragments together. The overlapping sequence of adjoining fragments is much longer than those used in Golden Gate Assembly, and therefore results in a higher percentage of correct assemblies.

Also provided herein are vectors comprising engineered nucleic acids. A vector is a nucleic acid (e.g., DNA) used as a vehicle to artificially carry genetic material (e.g., an engineered nucleic acid) into another cell where, for example, it can be replicated and/or expressed. In some embodiments, a vector is an episomal vector (see, e.g., Van Craenenbroeck K. et al. Eur. J. Biochem. 267, 5665, 2000, incorporated by reference herein). A non-limiting example of a vector is a plasmid. Plasmids are double-stranded generally circular DNA sequences that are capable of automatically replicating in a host cell. Plasmid vectors typically contain an origin of replication that allows for semi-independent replication of the plasmid in the host and also the transgene insert. Plasmids may have more features, including, for example, a multiple cloning site, which includes nucleotide overhangs for insertion of a nucleic acid insert, and multiple restriction enzyme consensus sites to either side of the insert. Another non-limiting example of a vector is a viral vector.

A nucleic acid, in some embodiments, comprises a promoter operably linked to a nucleotide sequence encoding the recombinase. A promoter is a control region of a nucleic acid sequence at which initiation and rate of transcription of the remainder of a nucleic acid sequence are controlled. A promoter may also contain sub-regions at which regulatory proteins and molecules may bind, such as RNA polymerase and other transcription factors. Promoters may be constitutive, inducible, activatable, repressible, tissue-specific or any combination thereof.

A promoter drives expression or drives transcription of the nucleic acid sequence that it regulates. Herein, a promoter is considered to be operably linked when it is in a correct functional location and orientation in relation to a nucleotide sequence it regulates to control (“drive”) transcriptional initiation and/or expression of that sequence.

A promoter may be one naturally associated with a gene or sequence, as may be obtained by isolating the 5′ non-coding sequences located upstream of the coding segment of a given gene or sequence. Such a promoter is referred to as an endogenous promoter.

In some embodiments, a coding nucleic acid sequence may be positioned under the control of a recombinant or heterologous promoter, which refers to a promoter that is not normally associated with the encoded sequence in its natural environment. Such promoters may include promoters of other genes; promoters isolated from any other cell; and synthetic promoters or enhancers that are not naturally occurring such as, for example, those that contain different elements of different transcriptional regulatory regions and/or mutations that alter expression through methods of genetic engineering that are known in the art. In addition to producing nucleic acid sequences of promoters and enhancers synthetically, sequences may be produced using recombinant cloning and/or nucleic acid amplification technology, including polymerase chain reaction (PCR) (see U.S. Pat. Nos. 4,683,202 and 5,928,906).

Contemplated herein, in some embodiments, are RNA pol II and RNA pol III promoters. Promoters that direct accurate initiation of transcription by an RNA polymerase II are referred to as RNA pol II promoters. Examples of RNA pol II promoters for use in accordance with the present disclosure include, without limitation, human cytomegalovirus promoters, human ubiquitin promoters, human histone H2A1 promoters and human inflammatory chemokine CXCL 1 promoters. Other RNA pol II promoters are also contemplated herein. Promoters that direct accurate initiation of transcription by an RNA polymerase III are referred to as RNA pol III promoters. Examples of RNA pol III promoters for use in accordance with the present disclosure include, without limitation, a U6 promoter, a H1 promoter and promoters of transfer RNAs, 5S ribosomal RNA (rRNA), and the signal recognition particle 7SL RNA.

Promoters of an engineered nucleic acids may be inducible promoters, which are promoters that are characterized by regulating (e.g., initiating or activating) transcriptional activity when in the presence of, influenced by or contacted by an inducer signal. An inducer signal may be endogenous or a normally exogenous condition (e.g., light), compound (e.g., chemical or non-chemical compound) or protein that contacts an inducible promoter in such a way as to be active in regulating transcriptional activity from the inducible promoter. An inducible promoter of the present disclosure may be induced by (or repressed by) one or more physiological condition(s), such as changes in light, pH, temperature, radiation, osmotic pressure, saline gradients, cell surface binding, and the concentration of one or more extrinsic or intrinsic inducing agent(s). Non-limiting examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells). Other inducible promoter systems are known in the art and may be used in accordance with the present disclosure.

An engineered nucleic acid, in some embodiments, comprises a gene of interest flanked by recombinase recognition sites. In some embodiments, the gene of interest is a marker gene encoding, for example, a detectable marker protein or a selectable marker protein. Examples of detectable marker proteins include, without limitation, fluorescent proteins (e.g., GFP, EGFP, sfGFP, TagGFP, Turbo GFP, AcGFP, ZsGFP, Emerald, Azami green, mWasabi, T-Sapphire, EBFP, EBFP2, Azurite, mTagBFP, ECFP, mECFP, Cerulean, mTurquoise, CyPet, AmCyanl, Midori-ishi Cyan, TagCFP, mTFP1, EYFP, Topaz, Venus, mCitrine, YPET, TagYFP, PhiYFP, ZsYellow1, mBanana, Kusabira Orange, Orange2, mOrange, mOrange2, dTomato, dTomato-Tandem, TagRFP, TagRFP-T, DsRed, DsRed2, DsRed-Express (T1), DsRed-Monomer, mTangerine, mRuby, mApple, mStrawberry, AsRed2, mRFP1, JRed, mCherry, HcRedl, mRaspberry, dKeima-Tandem, HcRed-Tandem, mPlum, AQ143 and variants thereof). Examples of selectable marker proteins include, without limitation, dihydrofolate reductase, glutamine synthetase, hygromycin phosphotransferase, puromycin N-acetyltransferase, and neomycin phosphotransferase.

Cells

Some aspects of the present disclosure provide cell comprising and/or expressing the engineered recombinase, engineered nucleic acid, and/or vector described herein. In some embodiments, engineered nucleic acids of the present disclosure are expressed in a broad range of cell types. In other embodiments, the recombinases and their cognate recognition site pairs are used to modify a broad range of cell types. In some embodiments, engineered nucleic acids are expressed in and/or the recombinases are used to modify plants cells, bacterial cells, yeast cells, insect cells, mammalian cells, or other types of cells. Any one of the foregoing types of cells may be transgenic cells.

Plants have been increasingly used as alternative recombinant protein expression system. There are three broad plant production systems: whole plant, culture of organized plant tissues and plant cell culture. All these three systems are able to produce recombinant proteins with complex glycosylation patterns and post-translational modification. Thus, plants and plant cells may be used to produce the recombinases described herein. Alternatively (or in addition), the recombinases and their cognate recognitions site pairs may be used to genetically modified plants (e.g., crops) used in agriculture, for example, to introduce a new trait to the plant.

Bacterial cells of the present disclosure include bacterial subdivisions of Eubacteria and Archaebacteria. Eubacteria can be further subdivided into gram-positive and gram-negative Eubacteria, which depend upon a difference in cell wall structure. Also included herein are those classified based on gross morphology alone (e.g., cocci, bacilli). In some embodiments, the bacterial cells are Gram-negative cells, and in some embodiments, the bacterial cells are Gram-positive cells. Examples of bacterial cells of the present disclosure include, without limitation, cells from Yersinia spp., Escherichia spp., Klebsiella spp., Acinetobacter spp., Bordetella spp., Neisseria spp., Aeromonas spp., Franciesella spp., Corynebacterium spp., Citrobacter spp., Chlamydia spp., Hemophilus spp., Brucella spp., Mycobacterium spp., Legionella spp., Rhodococcus spp., Pseudomonas spp., Helicobacter spp., Salmonella spp., Vibrio spp., Bacillus spp., Erysipelothrix spp., Salmonella spp., Streptomyces spp., Bacteroides spp., Prevotella spp., Clostridium spp., Bifidobacterium spp., or Lactobacillus spp. In some embodiments, the bacterial cells are from Bacteroides thetaiotaomicron, Bacteroides fragilis, Bacteroides distasonis, Bacteroides vulgatus, Clostridium leptum, Clostridium coccoides, Staphylococcus aureus, Bacillus subtilis, Clostridium butyricum, Brevibacterium lactofermentum, Streptococcus agalactiae, Lactococcus lactis, Leuconostoc lactis, Actinobacillus actinobycetemcomitans, cyanobacteria, Escherichia coli, Helicobacter pylori, Selnomonas ruminatium, Shigella sonnei, Zymomonas mobilis, Mycoplasma mycoides, Treponema denticola, Bacillus thuringiensis, Staphylococcus lugdunensis, Leuconostoc oenos, Corynebacterium xerosis, Lactobacillus plantarum, Lactobacillus rhamnosus, Lactobacillus casei, Lactobacillus acidophilus, Streptococcus spp., Enterococcus faecalis, Bacillus coagulans, Bacillus ceretus, Bacillus popillae, Synechocystis strain PCC6803, Bacillus liquefaciens, Pyrococcus abyssi, Selenomonas nominantium, Lactobacillus hilgardii, Streptococcus ferus, Lactobacillus pentosus, Bacteroides fragilis, Staphylococcus epidermidis, Zymomonas mobilis, Streptomyces phaechromogenes, or Streptomyces ghanaenis. Endogenous bacterial cells refer to non-pathogenic bacteria that are part of a normal internal ecosystem such as bacterial flora.

In some embodiments, bacterial cells of the disclosure are anaerobic bacterial cells (e.g., cells that do not require oxygen for growth). Anaerobic bacterial cells include facultative anaerobic cells such as, for example, Escherichia coli, Shewanella oneidensis and Listeria monocytogenes. Anaerobic bacterial cells also include obligate anaerobic cells such as, for example, Bacteroides and Clostridium species. In humans, for example, anaerobic bacterial cells are most commonly found in the gastrointestinal tract.

In some embodiments, the cells are mammalian cells. Non-limiting examples of mammalian cells include human cells, primate cells (e.g., vero cells), rat cells (e.g., GH3 cells, 0C23 cells), and mouse cells (e.g., MC3T3 cells). There are a variety of human cell lines, including, without limitation, human embryonic kidney (HEK) cells, HeLa cells, cancer cells from the National Cancer Institute's 60 cancer cell lines (NCI60), DU145 (prostate cancer) cells, Lncap (prostate cancer) cells, MCF-7 (breast cancer) cells, MDA-MB-438 (breast cancer) cells, PC3 (prostate cancer) cells, T47D (breast cancer) cells, THP-1 (acute myeloid leukemia) cells, U87 (glioblastoma) cells, SHSYSY human neuroblastoma cells (cloned from a myeloma) and Saos-2 (bone cancer) cells. In some embodiments, the cells are human embryonic kidney (HEK) cells (e.g., HEK 293 or HEK 293T cells). In some embodiments, the cells are stem cells (e.g., human stem cells) such as, for example, pluripotent stem cells (e.g., human pluripotent stem cells including human induced pluripotent stem cells (hiPSCs)). A stem cell is a cell with the ability to divide for indefinite periods in culture and to give rise to specialized cells. A pluripotent stem cell refers to a type of stem cell that is capable of differentiating into all tissues of an organism, but not alone capable of sustaining full organismal development. A human induced pluripotent stem cell refers to a somatic (e.g., mature or adult) cell that has been reprogrammed to an embryonic stem cell-like state by being forced to express genes and factors important for maintaining the defining properties of embryonic stem cells (see, e.g., Takahashi and Yamanaka, Cell 126 (4): 663-76, 2006, incorporated by reference herein). Human induced pluripotent stem cell cells express stem cell markers and are capable of generating cells characteristic of all three germ layers (ectoderm, endoderm, mesoderm).

Additional non-limiting examples of cell lines that may be used in accordance with the present disclosure include 293-T, 293-T, 3T3, 4T1, 721, 9L, A-549, A172, A20, A253, A2780, A2780ADR, A2780cis, A431, ALC, B16, B35, BCP-1, BEAS-2B, bEnd.3, BHK-21, BR 293, BxPC3, C2C12, C3H-10T1/2, C6, C6/36, Cal-27, CGR8, CHO, CML T1, CMT, COR-L23, COR-L23/5010, COR-L23/CPR, COR-L23/R23, COS-7, COV-434, CT26, D17, DH82, DU145, DuCaP, E14Tg2a, EL4, EM2, EM3, EMT6/AR1, EMT6/AR10.0, FM3, H1299, H69, HB54, HB55, HCA2, Hepa1c1c7, High Five cells, HL-60, HMEC, HT-29, HUVEC, J558L cells, Jurkat, JY cells, K562 cells, KCL22, KG1, Ku812, KYO1, LNCap, Ma-Mel 1, 2, 3 . . . 48, MC-38, MCF-10A, MCF-7, MDA-MB-231, MDA-MB-435, MDA-MB-468, MDCK II, MG63, MONO-MAC 6, MOR/0.2R, MRCS, MTD-1A, MyEnd, NALM-1, NCI-H69/CPR, NCI-H69/LX10, NCI-H69/LX20, NCI-H69/LX4, NIH-3T3, NW-145, OPCN/OPCT Peer, PNT-1A/PNT 2, PTK2, Raji, RBL cells, RenCa, RIN-5F, RMA/RMAS, S2, Saos-2 cells, Sf21, Sf9, SiHa, SKBR3, SKOV-3, T-47D, T2, T84, THP1, U373, U87, U937, VCaP, WM39, WT-49, X63, YAC-1 and YAR cells.

Cells of the present disclosure, in some embodiments, are engineered (e.g., genetically modified). An engineered cell contains an exogenous nucleic acid or a nucleic acid that does not occur in nature (e.g., a modified nucleic acid). In some embodiments, an engineered cell contains a mutation in a genomic nucleic acid. In some embodiments, an engineered cell contains an exogenous independently replicating nucleic acid (e.g., an engineered nucleic acid present on an episomal vector). In some embodiments, an engineered cell is produced by introducing a foreign or exogenous nucleic acid (e.g., expressing a recombinase) into a cell. A nucleic acid may be introduced into a cell by conventional methods, such as, for example, electroporation (see, e.g., Heiser W. C. Transcription Factor Protocols: Methods in Molecular Biology™ 2000; 130: 117-134), chemical (e.g., calcium phosphate or lipid) transfection (see, e.g., Lewis W. H., et al., Somatic Cell Genet. 1980 May; 6(3): 333-47; Chen C., et al., Mol Cell Biol. 1987 August; 7(8): 2745-2752), fusion with bacterial protoplasts containing recombinant plasmids (see, e.g., Schaffner W. Proc Natl Acad Sci USA. 1980 April; 77(4): 2163-7), transduction, conjugation, or microinjection of purified DNA directly into the nucleus of the cell (see, e.g., Capecchi M. R. Cell. 1980 November; 22(2 Pt 2): 479-88).

In some embodiments, a cell is modified to express a reporter molecule. In some embodiments, a cell is modified to express an inducible promoter operably linked to a reporter molecule (e.g., a fluorescent protein such as green fluorescent protein (GFP) or other reporter molecule).

In some embodiments, a cell is modified to overexpress a recombinase (e.g., via introducing or modifying a promoter or other regulatory element near the endogenous gene that encodes the recombinase to increase its expression level). In some embodiments, a cell is modified by site-specific recombination using the molecules identified herein.

In some embodiments, an engineered nucleic acid construct may be codon-optimized, for example, for expression in mammalian cells (e.g., human cells) or other types of cells. Codon optimization is a technique to maximize the protein expression in living organism by increasing the translational efficiency of gene of interest by transforming a DNA sequence of nucleotides of one species into a DNA sequence of nucleotides of another species. Methods of codon optimization are well-known.

Engineered nucleic acid constructs of the present disclosure may be transiently expressed or stably expressed. Transient cell expression refers to expression by a cell of a nucleic acid that is not integrated into the nuclear genome of the cell. By comparison, stable cell expression refers to expression by a cell of a nucleic acid that remains in the nuclear genome of the cell and its daughter cells. Typically, to achieve stable cell expression, a cell is co-transfected with a marker gene and an exogenous nucleic acid (e.g., engineered nucleic acid) that is intended for stable expression in the cell. The marker gene gives the cell some selectable advantage (e.g., resistance to a toxin, antibiotic, or other factor). Few transfected cells will, by chance, have integrated the exogenous nucleic acid into their genome. If a toxin, for example, is then added to the cell culture, only those few cells with a toxin-resistant marker gene integrated into their genomes will be able to proliferate, while other cells will die. After applying this selective pressure for a period of time, only the cells with a stable transfection remain and can be cultured further. Examples of marker genes and selection agents for use in accordance with the present disclosure include, without limitation, dihydrofolate reductase with methotrexate, glutamine synthetase with methionine sulphoximine, hygromycin phosphotransferase with hygromycin, puromycin N-acetyltransferase with puromycin, and neomycin phosphotransferase with Geneticin, also known as G418. Other marker genes/selection agents are contemplated herein.

Expression of nucleic acids in transiently-transfected and/or stably-transfected cells may be constitutive or inducible. Inducible promoters for use as provided herein are described above.

Some aspects of the present disclosure provide cells that comprises 1 to 10 engineered nucleic acids (e.g., engineered nucleic acids encoding recombinases). In some embodiments, a cell comprises 1, 2, 3, 4, 5, 6, 7, 8, 9, 10 or more engineered nucleic acids. It should be understood that a cell that comprises an engineered nucleic acid is a cell that comprises copies (more than one) of an engineered nucleic acid. Thus, a cell that comprises at least two engineered nucleic acids is a cell that comprises copies of a first engineered nucleic acid and copies of a second engineered nucleic acid, wherein the first engineered nucleic acid is different from the second engineered nucleic acid. Two engineered nucleic acids may differ from each other with respect to, for example, sequence composition (e.g., type, number and arrangement of nucleotides), length, or a combination of sequence composition and length.

Some aspects of the present disclosure provide cells that comprises 1 to 10 episomal vectors, or more, each vector comprising, for example, an engineered nucleic acids (e.g., engineered nucleic acids encoding gRNAs). In some embodiments, a cell comprises 1, 2, 3, 4, 5, 6, 7, 8, 9, 10 or more vectors.

Also provided herein, in some aspects, are methods that comprise introducing into a cell an (e.g., at least one, at least two, at least three, or more) engineered nucleic acid or an episomal vector (e.g., comprising an engineered nucleic acid). As discussed elsewhere herein, an engineered nucleic acid may be introduced into a cell by conventional methods, such as, for example, electroporation, chemical (e.g., calcium phosphate or lipid) transfection, fusion with bacterial protoplasts containing recombinant plasmids, transduction, conjugation, or microinjection of purified DNA directly into the nucleus of the cell.

In some embodiments, a cell comprises a genomic sequence flanked by recombinase recognition sites cognate to the engineered recombinase.

Animal Models

Some aspects of the present disclosure provide animal models comprising cells expressing a recombinase described herein. Other aspects provide methods of producing animal models using the recombinases and cognate recognition site pairs described herein. In some embodiments, an animal model is a rodent model, such as a rat model or a mouse model. In some embodiments, an animal model is a primate model.

Computer Implementation

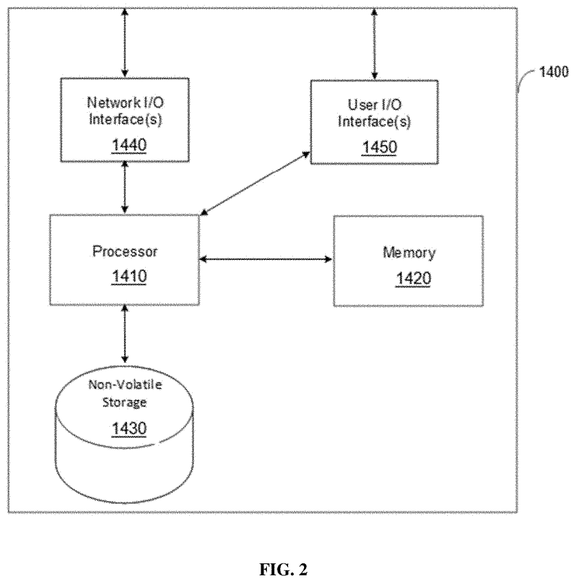

Some aspects of the present disclosure provide a computer implemented process. For example, at least some of the steps of the methods described herein (e.g., FIG. 1) may be implemented in software and carried out by a computing device. The software can be written in any suitable programming language and stored on any suitable recording medium including a computing system hard drive, computing system local memory, a computing network server, a cloud storage, and/or any computer readable medium. In an embodiment, the software may include an artificial intelligence machine learning algorithm, trained on initial data, which learns as more data is fed into the system. The method may be performed by any hardware processor capable of implementing the software steps, such as that of a general purpose computer, as illustrated in block diagram form in FIG. 2.

In some embodiments, a computer implemented method comprises: mining from a protein database putative recombinase sequences based on conserved recombinase domain architecture or other measure of homology to known recombinases; linking the putative recombinase sequences to prokaryotic genomic sequences containing their corresponding coding sequences; scanning those genomic sequences to identify prophage sequences containing the coding sequences; aligning the prophage sequences and their boundary-flanking sequences with homologous genomic sequences from the same genus to produce sequence alignments; and automatically solve for putative cognate recombinase recognition sites by detecting overlapping sequences in the sequence alignments.

In some embodiments, the mining is based on a precisely ordered recombinase domain superfamily architecture or other measure of homology to known recombinases.

In some embodiments, the linking includes accessing a database that comprises annotated records of genomes assembled from long-read nucleotide sequences, short-read nucleotide sequences, or a combination of long- and short-read nucleotide sequences, or directly annotated records of long-read nucleotide sequences.

In some embodiments, the linking includes automatically removing uninformative nucleotide sequences from the genomic coding sequences.

In some embodiments, the genomic coding sequences includes at least 2, at least 5, at least 10, at least 25, at least 50, or at least 100 annotated genomic coding sequences.

In some embodiments, the flanking boundary sequences have a length of at least 20 kilobases.

In some embodiments, the automatically solving includes defining multiple putative cognate recombinase recognition sites for a single recombinase.

In some embodiments, the method further comprises verifying that all putative cognate recombinase recognition sites solved flank a sequence encoding at least one of the putative recombinase sequences.

In an embodiment, the putative recombinase sequences comprise tyrosine and/or serine recombinase, the serine recombinase sequences comprise resolvase and/or integrase sequences.

Some aspects of the present disclosure provide a computer readable medium on which is stored a computer program which, when implemented by a computer processor, causes the processor to: mine from a protein database putative recombinase sequences based on conserved recombinase domain architecture or other measure of homology to known recombinases; link the putative recombinase sequences to prokaryotic genomic sequences containing their corresponding coding sequences; scan those genomic sequences to identify prophage sequences containing the coding sequences; align the prophage sequences and their boundary-flanking sequences with homologous genomic sequences from the same genus to produce sequence alignments; and automatically solve for putative cognate recombinase recognition sites by detecting overlapping sequences in the sequence alignments.

FIG. 1 is a flow chart of an illustrative process for discovering recombinases and cognate recognition site pairs, in accordance with some embodiments of the technology described herein. The process may be performed on any suitable computing device(s) (e.g., a single computing device, multiple computing devices co-located in a single physical location or located in multiple physical locations remote from one another, one or more computing devices part of a cloud computing system, etc.), as aspects of the technology described herein are not limited in this respect.

Step 1 includes identifying putative homologs of recombines genes by precise ordering of conserved domains (domain architecture). Step 2 includes retrieving putative recombinase coding sequence(s) in sequence database(s). Step 3 includes detecting prophages containing the putative recombinase coding sequence(s) within genomic region(s) and extracting these sequences with long flanking regions (allowing for an error-margin in prophage coordinate prediction). Step 4 (optionally designed for automation) includes aligning the extracted sequences against reference genomes and identifying genomic homologs that lack prophages, and optionally a broad secondary search for enhanced discovery. Steps 5 and 6 include automatically searching for overlaps between left and right prophage alignment ranges to identify putative core region(s) of recombinase substrates (Step 5), and solving for complete cognate recombination sites, while reporting confidence measures, handling ambiguity, and including multiple quality control steps (Step 6). Steps 1-6 may be implemented in a continuous scanning mode whereby sequencing databases are accessed routinely and the results refreshed based on newly reported/deposited sequences.

An illustrative implementation of a computer system 1400 that may be used in connection with any of the embodiments of the technology described herein is shown in FIG. 2. The computer system 1400 includes one or more processors 1410 and one or more articles of manufacture that comprise non-transitory computer-readable storage media (e.g., memory 1420 and one or more non-volatile storage media 1430). The processor 1410 may control writing data to and reading data from the memory 1420 and the non-volatile storage device 1430 in any suitable manner, as the aspects of the technology described herein are not limited in this respect. To perform any of the functionality described herein, the processor 1410 may execute one or more processor-executable instructions stored in one or more non-transitory computer-readable storage media (e.g., the memory 1420), which may serve as non-transitory computer-readable storage media storing processor-executable instructions for execution by the processor 1410.

Computing device 1400 may also include a network input/output (I/O) interface 1440 via which the computing device may communicate with other computing devices (e.g., over a network), and may also include one or more user I/O interfaces 1450, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The above-described embodiments can be implemented in any of numerous ways. For example, the embodiments may be implemented using hardware, software or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-discussed functions of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

Applications

One application of the present disclosure includes natural recombinase:recognition site pair discovery for training a machine learning model that learns the relationship between a recombinase's amino acid sequence and the DNA substrates it recognizes and recombines. The generation of engineered (re-programmed) recombinases that recombine at DNA targets not previously known to be targeted in nature is a long-standing challenge in protein design. Prior to the implementation of the present method, there were not enough examples from nature for a machine learning model of recombinase:recognition site pair to be successfully trained. However, as this continuously-operating, fully-automated method discovers new, naturally occurring recombinase:recognition site pairs, it is assembling a training set from nature that is indeed big enough to train a machine learning algorithm on this dataset. This model could then be used to predict the amino acid sequence of one or more candidate recombinase enzymes that would recognize arbitrary DNA targets of a user's choosing. The model could also be used to predict the amino acid sequence of a recombinase that would avoid and have no activity on one or more arbitrary DNA targets of a user's choosing. Machine-generated predictions may be explicitly tested such that an empirical target specificity profile and/or quantitative recombinase assay measurement is gathered for each machine-generated recombinase sequence. Empirical data describing the activity of machine-generated recombinases on recognition site pairs of interest may be use to further train and refine the model. In this manner, over iterative cycles of (i) prediction, and (ii) experimentation, the model's performance will be enhanced such that it can make increasingly accurate and predictions of recombinase amino acid sequences that have high specificity for a recognition site of interest. In some embodiments, the aforementioned machine learning model that predicts new recombinase sequences is a generative model that is informed, at least in part, by the three-dimensional structure of a recombinase enzyme, or recombinase enzyme sub-type (e.g. large phage serine integrase), such that newly predicted sequences have increased likelihood of folding into a recombinase-like structure and therefore, having recombinase-like function.

Another application of the present disclosure includes identifying ideal starting protein variants for directed evolution of re-programmable recombinases. The generation of engineered (re-programmed) recombinases that recombine at DNA targets not previously known to be targeted in nature is a long-standing challenge in protein design. Prior to the implementation of the present method, practitioners of directed evolution for recombinases performed directed evolution on a small number of site-specific recombinases, regardless of how far their native sequences deviated from the desired target sequence. The more divergent a target sequence is from the native sequence on which a recombinase has activity, the more arduous engineering is likely required to reprogram the DNA recognition. Therefore, generation of a long list of natural recombinase:recognitoin site pairs offers more flexibility in that one may choose a natural recombinase with a target site as close as possible to a desirable site, necessitating less engineering during reprogramming.

Yet another application of the present disclosure includes modifying the genome of cells using any of the engineered recombinases described herein.

Kits

Some aspects of the present disclosure provide kits. The kits may comprise, for example, an engineered recombinase, engineered nucleic acid, and/or vector described herein. In some embodiments, the kits further comprise a cell transfection reagent.

The kits described herein may include one or more containers housing components for performing the methods described herein and optionally instructions of uses. Kits for research purposes may contain the components in appropriate concentrations or quantities for running various experiments. Any of the kits described herein may further comprise components needed for performing the methods.

Each components of the kits, where applicable, may be provided in liquid form (e.g., in solution), or in solid form, (e.g., a dry powder). In certain cases, some of the components may be lyophilized, reconstituted, or processed (e.g., to an active form), for example, by the addition of a suitable solvent or other species (for example, water or certain organic solvents), which may or may not be provided with the kit.

In some embodiments, the kits may optionally include instructions and/or promotion for use of the components provided. Instructions can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which can also reflect approval by the agency of manufacture, use or sale for animal administration. As used herein, “promoted” includes all methods of doing business including methods of education, hospital and other clinical instruction, scientific inquiry, drug discovery or development, academic research, pharmaceutical industry activity including pharmaceutical sales, and any advertising or other promotional activity including written, oral and electronic communication of any form, associated with the invention. Additionally, the kits may include other components depending on the specific application, as described herein.

The kits may contain any one or more of the components described herein in one or more containers. The components may be prepared sterilely, packaged in syringe and shipped refrigerated. Alternatively, it may be housed in a vial or other container for storage. A second container may have other components prepared sterilely. Alternatively, the kits may include the active agents premixed and shipped in a vial, tube, or other container.

The kits may have a variety of forms, such as a blister pouch, a shrink wrapped pouch, a vacuum sealable pouch, a sealable thermoformed tray, or a similar pouch or tray form, with the accessories loosely packed within the pouch, one or more tubes, containers, a box or a bag. The kits may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kits may also include other components, depending on the specific application, for example, containers, cell media, salts, buffers, reagents, syringes, needles, a fabric, such as gauze, for applying or removing a disinfecting agent, disposable gloves, a support for the agents prior to administration etc.

### ADDITIONAL EMBODIMENTS

Additional embodiments of the present disclosure are encompassed by the following numbered paragraphs.

1. A method comprising:

mining from a protein database putative recombinase sequences based on conserved recombinase domain architecture or other measure of homology to known recombinases;

linking the putative recombinase sequences to prokaryotic genomic sequences containing their corresponding coding sequences;

scanning those genomic sequences to identify prophage sequences containing the coding sequences;

aligning the prophage sequences and their boundary-flanking sequences with homologous genomic sequences, optionally, from the same genus to produce sequence alignments; and

automatically solving for putative cognate recombinase recognition sites by detecting overlapping sequences in the sequence alignments, thereby producing a solved recombinase list.

2. The method of paragraph 1, wherein the mining is based on a precisely ordered recombinase domain superfamily architecture or other measure of homology to known recombinases.

3. The method of paragraph 1 or 2, wherein the linking includes accessing a database that comprises annotated records of genomes assembled from long-read nucleotide sequences, short-read nucleotide sequences, or a combination of long- and short-read nucleotide sequences, or directly annotated records of long-read nucleotide sequences.

4. The method of any one of the preceding paragraphs, wherein the linking includes automatically removing uninformative nucleotide sequences from the genomic coding sequences.

5. The method of any one of the preceding paragraphs, wherein the genomic coding sequences includes at least 2, at least 5, at least 10, at least 25, at least 50, or at least 100 annotated genomic coding sequences.

6. The method of any one of the preceding paragraphs, wherein the boundary-flanking sequences have a length of at least 20 kilobases.

7. The method of any one of the preceding paragraphs, wherein the automatically solving includes defining multiple putative cognate recombinase recognition sites for a single recombinase.

8. The method of any one of the preceding paragraphs, wherein the automatically solving includes implementation of an algorithm that includes a measure of confidence in each predicted recombinase recognition site set, optionally in the form of ambiguity scores.

9. The method of any one of the preceding paragraphs, further comprising verifying that all putative cognate recombinase recognition sites solved flank a sequence encoding at least one of the putative recombinase sequences.

10. The method of any one of the preceding paragraphs, wherein the putative recombinase sequences comprise tyrosine and/or serine recombinase sequences.

11. The method of paragraph 10, wherein the serine recombinase sequences comprise resolvase and/or integrase sequences.

12. The method of any one of the preceding paragraphs, wherein the method is a computer-implemented method.

13. The method of any one of the preceding paragraphs, wherein the entirety of the method is automated.

14. The method of any one of the preceding paragraphs, further comprising continuously updating the solved recombinase list as the protein database is updated.

15. A computer readable medium on which is stored a computer program which, when implemented by a computer processor, causes the processor to:

mine from a protein database putative recombinase sequences based on conserved recombinase domain architecture or other measure of homology to known recombinases;

link the putative recombinase sequences to prokaryotic genomic sequences containing their corresponding coding sequences;

scan those genomic sequences to identify prophage sequences containing the coding sequences;

align the prophage sequences and their boundary-flanking sequences with homologous genomic sequences from the same genus to produce sequence alignments; and

solve for putative cognate recombinase recognition sites by detecting overlapping sequences in the sequence alignments.

16. The computer readable medium of paragraph 15, wherein the mining is based on a precisely ordered recombinase domain superfamily architecture or other measure of homology to known recombinases.

17. The computer readable medium of paragraph 15 or 16, wherein the linking includes accessing a database that comprises annotated records of genomes assembled from long-read nucleotide sequences, short-read nucleotide sequences, or a combination of long- and short-read nucleotide sequences, or directly annotated records of long-read nucleotide sequences.

18. The computer readable medium of any one of paragraphs 15-17, wherein the linking includes automatically removing uninformative nucleotide sequences from the genomic coding sequences.

19. The computer readable medium of any one of paragraphs 15-18, wherein the genomic coding sequences includes at least 2, at least 5, at least 10, at least 25, at least 50, or at least 100 annotated genomic coding sequences.

20. The computer readable medium of any one of paragraphs 15-19, wherein the boundary-flanking sequences have a length of at least 20 kilobases.

21. The computer readable medium of any one of paragraphs 15-20, wherein the solving includes defining multiple putative cognate recombinase recognition sites for a single recombinase.

22. The computer readable medium of any one of paragraphs 15-21, wherein the solving includes implementation of an algorithm that includes a measure of confidence in each predicted recombinase recognition site set, optionally in the form of ambiguity scores.

23. The computer readable medium of any one of paragraphs 15-22, further comprising verifying that all putative cognate recombinase recognition sites solved flank a sequence encoding at least one of the putative recombinase sequences.

24. The computer readable medium of any one of paragraphs 15-23, wherein the putative recombinase sequences comprise tyrosine and/or serine recombinase sequences.

25. The computer readable medium of paragraph 24, wherein the serine recombinase sequences comprise resolvase and/or integrase sequences.

26. The computer readable medium of any one of paragraphs 15-25, further comprising continuously updating the solved recombinase list as the protein database is updated.

27. A system configured to perform:

mining a protein database putative recombinase sequences based on conserved recombinase domain architecture or other measure of homology to known recombinases;

linking the putative recombinase sequences to prokaryotic genomic sequences containing their corresponding coding sequences;

scanning those genomic sequences to identify prophage sequences containing the coding sequences;

aligning the prophage sequences and their boundary-flanking sequences with homologous genomic sequences from the same genus to produce sequence alignments; and

solving for putative cognate recombinase recognition sites by detecting overlapping sequences in the sequence alignments.

28. The system of paragraph 27, wherein the system is a computer system.

29. The system of paragraph 27 or 28, wherein the mining is based on a precisely ordered recombinase domain superfamily architecture or other measure of homology to known recombinases.

30. The system of any one of paragraphs 27-29, wherein the linking includes accessing a database that comprises annotated records of genomes assembled from long-read nucleotide sequences, short-read nucleotide sequences, or a combination of long- and short-read nucleotide sequences, or directly annotated records of long-read nucleotide sequences.

31. The system of any one of paragraphs 27-30, wherein the linking includes automatically removing uninformative nucleotide sequences from the genomic coding sequences.

32. The system of any one of paragraphs 27-31, wherein the genomic coding sequences includes at least 2, at least 5, at least 10, at least 25, at least 50, or at least 100 annotated genomic coding sequences.

33. The system of any one of paragraphs 27-32, wherein the boundary-flanking sequences have a length of at least 20 kilobases.

34. The system of any one of paragraphs 27-33, wherein the solving includes defining multiple putative cognate recombinase recognition sites for a single recombinase.

35. The system of any one of paragraphs 27-34, wherein the solving includes implementation of an algorithm that includes a measure of confidence in each predicted recombinase recognition site set, optionally in the form of ambiguity scores.

36. The system of any one of paragraphs 27-35, further comprising verifying that all putative cognate recombinase recognition sites solved flank a sequence encoding at least one of the putative recombinase sequences.

37. The system of any one of paragraphs 27-36, wherein the putative recombinase sequences comprise tyrosine and/or serine recombinase sequences.

38. The system of paragraph 37, wherein the serine recombinase sequences comprise resolvase and/or integrase sequences.

39. The system of any one of paragraphs 27-38, further comprising continuously updating the solved recombinase list as the protein database is updated.

### EXAMPLES

### Example 1. Discovery of Large Serine Phage Integrases

While this example describes a method for identifying large serine phage integrases, it should be understood that the method may be used to identify other site-specific recombinases.

Step 1: A Conserved Domain superfamily sub-architecture common to all characterized Large Serine Phage Integrases was manually defined by performing an NCBI Conserved Domain (CD) search (http://www.ncbi.nlm nih.gov/Structure/cdd/wrpsb.cgi) on their amino acid sequences with default parameters (E<0.01) and deducing the largest consecutive Conserved Domain superfamily subarchitecture shared by them all. The largest common consecutive Conserved Domain superfamily subarchitecture (N-terminus to C-terminus direction) is: [\hat{ }]˜[c102788(Ser_Recombinase superfamily)]˜[c106512(Recombinase superfamily)], where [\hat{ }] denotes that no other Conserved Domain occurs N-terminal to c102788. The region C-terminal to c106512 is free to contain any number and combination of Conserved Domain superfamilies, or none at all.

The Accession.version identifiers of putative Large Serine Phage Integrase proteins in the NCBI Entrez non-redundant (nr) Protein Database are manually retrieved for each unique CDART architecture based on the Conserved Domain superfamily sub-architecture defined, using NCBI's CDART (http://www.ncbi.nlm nih.gov/ Structure/lexington/lexington.cgi) with default parameters, and concatenated together.

Step 2: Records of all nucleotide sequences encoding all putative Large Serine Phage Integrase proteins identified in Step 1 are retrieved as Identical Protein Groups (IPG) Records. For each unique protein sequence, this record details, for every annotated occurrence in the NCBI Entrez Nucleotide database of a coding sequence for the protein, the: unique IPG identifier of the protein sequence, the accession.version of the nucleotide record containing the coding sequence, the source database of this nucleotide record, the start and stop coordinates of the protein coding sequence within the whole nucleotide sequence, the strand encoding the protein (+/−), the accession.version of the protein record linked to this particular coding sequence occurrence, the protein name in the protein record linked to this particular coding sequence occurrence, the organism and strain linked to the nucleotide record containing the coding sequence, and the accession.version of the nucleotide Assembly record linked to the nucleotide record containing the coding sequence. This is achieved with the NCBI Entrez E-utlities command, EFetch, with db as “protein”, id as [a putative Large Serine Phage Integrase protein accession.version] and retype as “ipg”. By retrieving every annotated occurrence of a nucleotide sequence coding for each protein, (1) the chances of finding each putative Large Serine Phage Integrase gene in at least one genetic context that allows its associated att sites to be solved are increased, and (2) it becomes possible to independently solve associated att sites for a single Large Serine Phage Integrase protein found encoded in several genomic contexts, providing “biological replicates” and so information as to the specificity of an integrase for its attB and attP sites, for example.

Rows in the IPG record tables in which a nucleotide record is absent (Nucleotide Accession=“N/A”), or in which the nucleotide sequence is annotated as deriving from sources unlikely to yield attL/attR sites (e.g., artificial sequences, un-integrated plasmids, un-integrated phages), are removed to avoid wasteful downstream computation. Artificial sequences and un-integrated phages can be identified by string-searching the Organism column of the IPG record tables for the words “synthetic” or “artificial”, and “phage” or “virus”, respectively. Nucelotide sequences derived from plasmids may be identified by retrieving the Document Summary of the remaining Nucleotide records (NCBI Entrez E-utlities command, EFetch, with db as nuccore, id as the Nucleotide record accession.version, and retype as docsum), and string-searching the Document Summary Title field for the word “plasmid”. Note, there are other ways to restrict the IPG record table rows to exclude all nucleotide records coming from undesired/unuseful sources. By using methods that enable automatic removal of uninformative nucleotide sequences, including artificial/synthetic nucleotide sequences, from the search list, which can be common for classes of proteins such as integrases, speed and automation are added to the pipeline.

After this filtering step, the remaining nucleic acid sequences named in the IPG record tables are uniqued on their accession.version identifiers and scanned to detect the presence and approximate location of any putative prophages. This is achieved within the script by accessing the web-based Phaster program, through their URL API, with built-in pause times and error-handling to avoid crashes due to download failures. The input submitted to Phaster is the nucleotide's accession.version, rather than the nucleotide sequence itself, allowing pre-computed Phaster records associated to certain NCBI Entrez nucleotide accession.versions to be instantly retrieved, and avoiding the need to download the nucleotide sequences pre-prophage-screening. The loop used to submit this set of Entrez accession.version-identified jobs to Phaster may be continuously re-run, or after a suitable time-delay, until all jobs have returned a Phaster report (JSON format) containing a non-null “error” field or a “status” field containing “Complete”. Note, there are many other open-source prophage-detection programs that may be used for this purpose, both web-based and locally executable (in which case FASTA files containing all the unique nucleotide sequences named in the filtered IPG record tables need to be first downloaded to use as the input for the prophage-detection program, using the Entrez E-utlities command, EFetch, with db as “nuccore”, id as [the Nucleotide record accession.version], and retype as “fasta”), such as Prophage Hunter, Prophinder, Phast and PhiSpy.

Step 3: The set of Phaster (or other prophage-detection software) output files are parsed to extract all instances of predicted intact/active prophages along with their predicted approximate coordinates within the submitted nucleotide sequences. For each prophage, its coordinates are compared with the coordinates of the set of putative Large Serine Phage Integrases encoded within the same nucleotide sequence (as recorded in the IPG record tables). An error margin for the predicted prophage coordinates is permitted (e.g., 20 kilobases (kb) for each boundary), and if a putative Large Serine Phage Integrase coding sequence overlaps this extended putative prophage range, the putative prophage details (including nucleotide Entrez accession.version, prophage unique identifier and predicted prophage coordinates), are kept for the later steps (note there may be several unique predicted prophages within a given nucleotide sequence). The concept of an error-margin in the prediction of prophage coordinates is included, so that putative Large Serine Phage Integrase coding sequences that do not lie within the originally predicted prophage coordinates but may later be discovered to indeed lie within the precisely solved prophage coordinates are not prematurely discounted (many Large Serine Phage Integrase coding sequences may lie close to one end of a prophage, and phage-detection software is known to display large error in prophage boundary prediction).

The unique set of Entrez nucleotide accession.version identifiers containing this set of predicted prophages lying close to or coinciding with a putative Large Serine Phage Integrase coding sequence is computed and their associated nucleotide sequences are downloaded from NCBI, if not already present from Step 2 if a locally-executed prophage-detection program is used (Entrez E-utlities command, EFetch, with db as “nuccore”, id as [the Nucleotide record accession.version], and retype as “fasta”).

Independently, the BLAST-formatted NCBI Entrez nucleotide (nt) database is downloaded/updated. Also independently, the unique set of genera from which the nucleotide sequences containing the set of predicted prophages lying close to or coinciding with a putative Large Serine Phage Integrase coding sequence are derived are computed, by taking the first word of the associated Organism values. (All genus words then surrounded by square brackets are re-defined as “unclassified”, following NCBI taxonomy annotation rules). An alternative approach is retrieving the NCBI genus taxonomy id associated to each full Organism name. For each unique resulting genus, the set of accession.version identifiers of all whole-genome-derived sequences in the Entrez Nucleotide database ascribed to this genus are retrieved from NCBI, using the Entrez E-utlities commands, Esearch then Efetch, with db as “nuccore”, term as [(genus[Organism]) AND (complete genome[title] OR chromosome[title])], and retype as “acc”. Also independently, the set of accession.version identifiers of all whole-genome-derived sequences in the Entrez Nucleotide database ascribed to prokaryotes is retrieved from NCBI, using the Entrez E-utlities commands, Esearch then Efetch, with db as “nuccore”, term as [(bacteria[Filter] OR archaea[Filter]) AND (complete genome[title] OR chromosome[title])], and retype as “acc”. Other Entrez search strategies may also be used to the same effect. For each of these genus-specific accession.version lists, and the total prokaryotic accession.version list, an associated BLAST+ alias database of the Entrez nucleotide database (titled to identify the genus it is based on, or the fact that it contains sequences from prokaryotes in general) is then created using the NCBI BLAST+ blastdb_aliastool command.

When this has been accomplished, all unique predicted prophages are extracted along with a chosen length of flanking DNA sequence, and aligned against the appropriate subset of whole-genome-derived sequences from the NCBI nucleotide database. First, the DNA sequence centered on each predicted prophage, and including a defined length (for example, 20 kb) on each side, is extracted using the prophage coordinates predicted by the prophage-detection software along with the relevant downloaded nucleotide sequences. If the predicted prophage start coordinate is less than this length from the start of the nucleotide sequence, or the predicted prophage stop coordinate is less than this length from the end of the nucleotide sequence, then the left flank will extend only to the start of the nucleotide sequence, and the right flank will extend only to the end of the nucleotide sequence, respectively. Alternatively, circular nucleotide sequences may be identified through an Entrez search, and in these cases, the full-length flanks may be extracted by accounting for this circularity. The coordinates of the putative Large Serine Phage Integrase coding sequences and the predicted prophages within the extracted DNA sequences are recorded for future steps. Extracting long (e.g., at least 20 kb) flanks surrounding predicted prophages for alignment increases the success rate of solving precise prophage boundaries in Step 5, as the large error in prophage boundary prediction by prophage-detection software (exacerbated by prophage sequences sometimes being disrupted by other mobile elements) can result in the ends of the true prophage not being reached when shorter flanks are taken.

Step 4: Each unique extracted DNA sequence containing a predicted prophage is aligned against the appropriate subset of whole-genome-derived sequences from the NCBI Nucleotide ndatabase using the BLASTn command from the NCBI BLAST+software package. For an optimal balance of speed and sensitivity, the following parameters are used: -task MegaBLAST, -word_size 32, -evalue 0.1, -max_target_seqs 200, with -outfmt 6. The appropriate alias BLAST database to use as the reference set is determined by extracting the genus word associated to each predicted prophage instance, in precisely the same way as was done to compute the unique set of genera above. Predicted prophage-containing sequences ascribed to a genus for which a non-empty alias database was not successfully constructed are instead aligned against the all-prokaryote alias database, using the same parameters as for the genus-specific alignments. Cases in which an appropriate non-empty genus-specific alias database was successfully created but returned no hits in a BLAST search may be re-attempted using the all-prokaryote alias BLAST database as reference set, in case of, for example, taxonomy errors.

In Steps 3 and 4, a rapid, efficient, and scalable, automated strategy for alignment of predicted prophage-containing DNA sequences against whole-genome-derived reference sequences is provided. A non-redundant NCBI Entrez Nucleotide database may be used in combination with rapid Entrez search/fetch-enabled retrieval of the accession.version identifiers of all whole-genome/chromosomederived sequences for a desired genus (or all prokaryotes) within this nucleotide database and respective alias file creation. This in turn enables fast BLAST execution independent of the NCBI compute resources, during customized BLAST parameters may be utilized. Finally, these steps included a strategy to handle cases where genus-specific alignment searches fail, such as known/unknown taxonomic misclassification or a scarcity of sequenced genomes for a particular genus, by using a broader reference set (all whole-genome-derived prokaryotic sequences in the nucleotide database) for these cases. The more intensive computation necessitated by this larger reference set is made feasible by the methods provided herein.

Step 5: A custom algorithm is applied to automatically search for cases where predicted prophage-containing sequences have been aligned with partially homologous sequences lacking the prophage, and to use the alignment information to solve the putative att core sequence for the prophage in question. The putative core sequence may be ambiguous due to alignment details, in which case the most likely core sequence is recorded, possibly along with other potential core sequences and with an ambiguity score. Core sequences are used to infer putative attL and attR sites by taking a ˜66 bp region centered on the core sequence at the left and right ends of the prophage, respectively, and putative attB and attP sites are computed based on strand exchange between the cores of attL and attR. att sites are associated with the ambiguity score of their inferred core sequence. Multiple/all reported alignments are considered for each predicted prophage-containing sequence, resulting in the potential for multiple core/attL/attR/attB/attP site sets to be inferred for each putative prophage. As different reference sequences can result in different alignment details, this can result in some putative prophages being associated to both ambiguous and unambiguous sites (in which case unambiguous sites can be prioritized), and allows for assessment of confidence in the inferred att sites (for some putative prophages, different reference sequences may give rise to the same set of inferred att sites, while for others, there may be inconsistencies between sets inferred from different reference sequences). To avoid false positives, putative att sites are only solved for a given alignment if at least one of the putative Large Serine Phage Integrase coding sequences associated to the predicted prophage in question lies within the precise prophage boundaries defined by the left and right core sites.

Each non-empty alignment output table from Step 4 is read in and processed as follows: all individual alignment ranges shorter than a given length (e.g., 900 bp) can be discarded to reduce computation time; a list of reference sequences producing more than 1 (filtered) alignment range with the predicted prophage-containing sequence in question is computed; for each of these reference sequences, its alignment ranges with the predicted prophage-containing sequence in question are categorized as aligning to the left prophage boundary region, the right prophage boundary region, or neither and so are discarded (a prophage boundary prediction error-margin is again permitted, e.g., 6 kb, such that any alignment range who's right end stops before the predicted prophage start coordinate plus this error margin is categorized as aligning to the left prophage boundary region, and any alignment range who's left end starts after the predicted prophage stop coordinate minus this error margin is categorized as aligning to the right prophage boundary region); for all iso-oriented combinations of left/right prophage boundary region alignment ranges for which at least one of the associated putative Large Serine Phage Integrase coding sequences lies fully between them, an overlap length between them with respect to their reference sequence coordinates is computed; if this yields a single overlap with a length longer than lbp and less than an appropriate upper limit, e.g., 31 bp, then the precise overlapping regions of the predicted prophage-containing sequence are extracted as the “left overlap” and “right overlap”, according to the prophage boundary they come from (if multiple such overlaps are detected, the alignment with this particular reference sequence is deemed complex and is flagged for, e.g., later manual analysis); if the “left overlap” and “right overlap” are identical, their sequence is unambiguously defined as the att core sequence, but if they are not identical (due to one or both alignment ranges extending beyond the core site), the longest exact matching substring(s) between the “left overlap” and “right overlap” is taken as the most likely core sequence(s); an ambiguity score is attributed to core sequences, and the set of att sites based on them, depending on whether “left overlap” and “right overlap” were identical (0), “left overlap” and “right overlap” were non-identical but there was a single longest exact matching substring between them (1), or “left overlap” and “right overlap” were non-identical and there were multiple longest exact matching substrings between them (# longest exact matches); the coordinates of all putative left/right core pairs in the context of the original complete nucleic acid sequence containing the predicted prophage are recorded for later quality control steps (by referring to the coordinates of the region extracted in Step 4); putative attL and attR sites are computed from each putative core sequence, by extracting a ˜66 bp region centered on the core sequence at the left or right prophage boundary, respectively; putative attB and attP sites are reconstructed on the basis of strand exchange between the cores of attL and attR. The coordinates of the attL and attR cores are compared with the coordinates of all putative Large Serine Phage Integrase coding sequences located in the same original Entrez nucleotide record as the predicted prophage-containing sequence in question, and all integrase coding sequences falling within these cores are recorded as potentially acting on the inferred att sites.

Here, an efficient algorithm for solving att sites automatically is implemented, as well as providing an automatic measure of confidence in each predicted att site set, in the form of ambiguity scores. Related to this, also provided is a strategy to automatically handle cases where the sequences of a “left overlap” and “right overlap” are non-identical.

For each putative prophage, the method considers multiple/all pairs of “left overlap” and “right overlap” detected from the alignment output to potentially define a list of att core sequences associated to that prophage (along with an ambiguity score for each). This can help improve the best ambiguity score achieved for a given prophage's att sites, as some alignments of the same predicted prophage-containing sequence may provide less ambiguous information than others, as well as provide other information relating to the overall confidence in the inferred att sites of a given prophage (e.g., one may infer different att core sequences for a given prophage, but with each having an ambiguity score of 0, indicating a potential problem in the alignment analysis for this predicted prophage-containing sequence).

Also included in the method is an explicit, efficient verification that all att site sets solved enclose at least one coding sequence for a putative Large Serine Phage Integrase from the Step 2 list, by only considering for overlap analysis left- and right-prophage boundary alignment range pairs that enclose one.

Further, a single prophage may contain multiple Large Serine Phage Integrases, any one of which may have been responsible for the recombination reaction between the original phage's attP site and the attB site of the prokaryotic chromosome where it is now detected as having integrated. With no rapid informatic way to deduce which integrase was responsible for the integration reaction, it is advantageous to document that any inferred att sites for this prophage may be the substrate of any of the integrases contained within it. This is achieved automatically and rapidly by using the integrase coding sequence coordinates found in the IPG records tables.

Step 6: Another, non-homologous class of phage integrases, the Tyrosine Phage Integrases, may occur within a prophage with Large Serine Phage Integrases, and so also demand consideration as the integrase responsible for a given integration reaction. IPG records for putative Tyrosine Phage Integrases may be obtained using similar homology-based methods as those detailed in Steps 1-3 for Large Serine Phage Integrases (Conserved Domain Architecture, but also, e.g., BLAST/PSI-BLAST). The coordinates of all putative attL/attR core pairs are thus compared with coordinates of putative Tyrosine Phage Integrase coding sequences, as in Step 5 for putative Large Serine Phage Integrase coding sequences, and an integrase is again ascribed to an att site set if its coding sequence falls within those core sites. If a Tyrosine Phage Integrase was responsible for the integration, the inferred attB and attP sites are less likely to be valid, due to their different typical lengths between Large Serine and Tyrosine Phage Integrases. It should also be noted that integrase coding sequences may be disrupted upon integration, which raises a small possibility that the integration was catalyzed by an undetected integrase (these cases could be detected with a more thorough informatic search for split integrase coding sequences).

Continuous Operation: With all steps of the pipeline fully automated, the exponentially growing volume of public sequence data can be leveraged by employing it continuously. New sequence data may be used in three ways:

(1) Predicted prophage regions previously found to carry putative Large Serine Phage Integrase coding sequences within (or reasonably near) them in Step 4, but with currently unsolved or only ambiguous att sites (“unsolved prophages”) can be aligned against new reference sequences as they are made available. For this, the local NCBI nucleotide database may be automatically updated at a regular time interval (e.g., weekly, monthly) using NCBI's update_blastdb.pl script, and the unique set of genera from which the current set of “unsolved prophages” is derived can be automatically computed as described in Step 4. For each unique resulting genus, the set of accession.version identifiers of all new whole-genome-derived sequences in the Entrez Nucleotide database ascribed to this genus are retrieved from NCBI using the Esearch/Efetch strategy described in Step 4 but with the addition of searching the Publication Date field with a date range from the date of the last local update to the current date. The same can be done for the new total prokaryotic accession.version list, using the other search criteria described in Step 4. An associated set of BLAST+ alias database files can be created from these accession.version lists, which can then be used as the subject sets for BLAST alignment with the current set of “unsolved prophage” sequences, according to the method of Step 4, with the methods of Step 5 and Step 6 following on. The list of current “unsolved prophages” is updated after each such update.

(2) Putative Large Serine Phage Integrases that have been previously mined but for which no coding sequences have been found to occur within (or close to) a predicted prophage (“unplaced integrases”) can potentially be located in new genetic contexts. New coding sequence instances of these proteins can be continuously mined by retrieving IPG records for them at regular intervals and comparing them with the previous records to extract new row entries. Any new entries can then be automatically passed through the remainder of Steps 3-6. The lists of current “unplaced integrases” and “unsolved prophages” are updated after each such update.

(3) Finally, records for new putative Large Serine Phage Integrase proteins can be retrieved from the NCBI Entrez Protein database as they are made available and be automatically submitted to the entire pipeline described in Steps 3-6, as they are up until now completely unanalyzed. CDART does not currently enable automatic retrieval of proteins with defined architectures, but new putative Large Serine Phage Integrase proteins may be automatically mined by updating a local copy of the NCBI non-redundant Protein database at a regular time interval (using the update_blastdb.pl script as in (1)), and searching this database for homologs of the current list of putative Large Serine Phage Integrase sequences using e.g., BLAST or PSI-BLAST (alternatively, newly added non-redundant sequences can be automatically downloaded in FASTA format, formatted as a database for a higher-performance aligner, e.g., DIAMOND, and aligned with this instead). The list of current putative Large Serine Phage Integrases is updated after each such update, as are the lists of current “unsolved prophages” and “unplaced integrases”.

Examples 2-4 below include newly-identified site-specific recombinases and their four (4) cognate recognition sites. These recombinases and recognition sites are grouped according to a shared characteristic or feature. Each group represents a new category of recombinases that has not been previously identified, and thus expands the capability to preform site specific recombination of DNA in vitro, in cells, and in vivo.

### Example 2. New Recombinases Families Grouped by Shared Homology

Described herein is a database of 395 site-specific recombinase amino acid sequences, each associated with at least four predicted att DNA substrates (L, R, B, P), where 64 of these recombinase target site pairings were previously known, and 331 are newly identified and disclosed herein (Tables 1 and 2). Site-specific recombinases and their associated DNA target pairs for recombinases that differ substantially in amino acid sequence from known recombinases with known DNA target sites were identified by clustering at 30% amino acid protein identity.

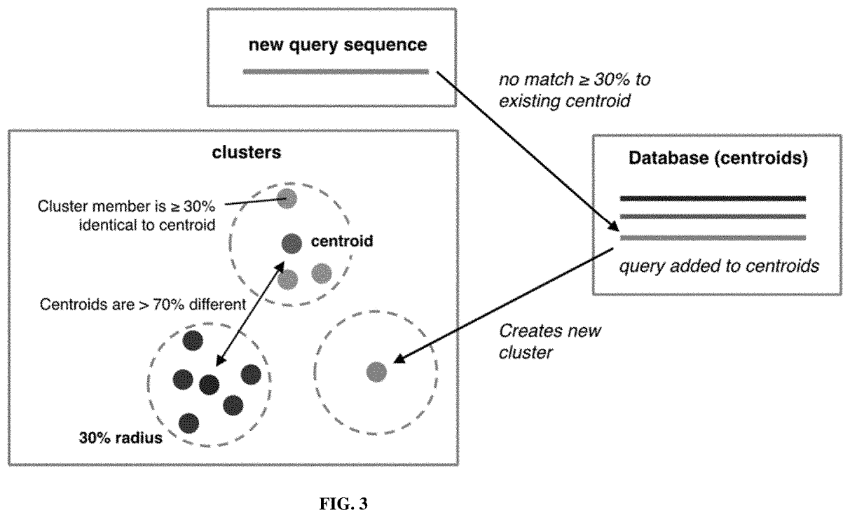

Clustering these sequences at 30% amino acid identity reveals 88 clusters. Within each of the 88 clusters, the member sequences share more than some threshold degree of homology at the amino acid level to the cluster's centroid—that threshold has been set to be 30%. All members to a given cluster are closer in homology space to their assigned cluster centroid than to any other cluster centroid. This means that cluster centroids are more than 70% different relative to each other (FIG. 3).

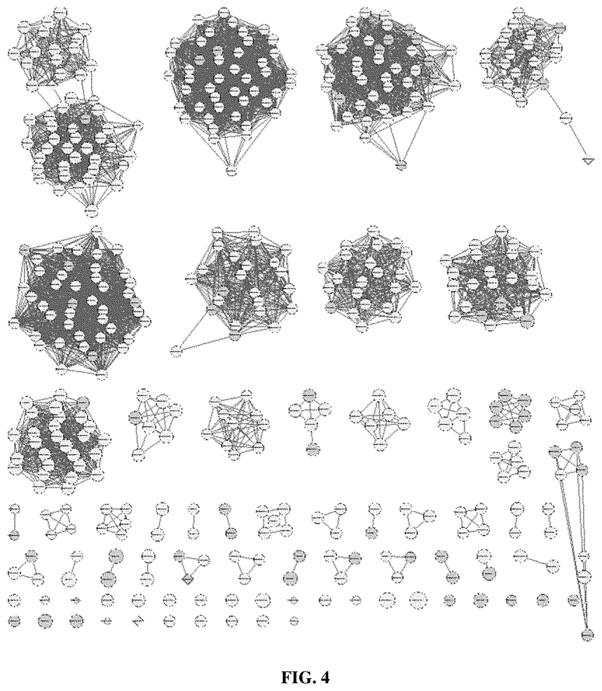

Of the 88 identified clusters, 51 clusters are entirely new—meaning that they do not contain any known recombinase genes that have previously described target sites (see FIG. 4). Each new site-specific recombinase cluster represents a new family of recombinases that is only distantly related (in homology space) to known enzymes. Each of these clusters represents therefore a new region of both recombinase and DNA target site sequence space.

The 110 new site-specific recombinases that together comprise 51 newly identified clusters (with no previously known site-solved members) along with their target sites are provided in Tables 1 and 2 (“New Recombinases” or “New R” indicated). Each centroid (“Cent”) can represent the entire cluster, as all clustered sequences are more than 30% similar to the centroid sequence.

TABLE 1
Recombinases and cognate recognition sites

| Protein Accession | SEQ | | | | | | Predicted Recognition Sites⁺ | | | |
|---|---|---|---|---|---|---|---|---|---|---|
| Number | ID NO: | Organism | C | New C | Cent | New R | L | R | B | P |
| | | | | | | | SEQ ID NO: | | | |
| AAD26564.1 | 1 | Enterococcus phage | 65 | No | No | No | | | | |
| | | phiFC1 | | | | | | | | |
| AAG59740.1 | 2 | Mycobacterium virus | 12 | No | No | No | | | | |
| | | Bxb1 | | | | | | | | |
| ABC40426.1 | 3 | Bacillus virus Wbeta | 49 | No | No | No | | | | |
| ADF59162.1 | 4 | Bacillus phage phi105 | 59 | No | No | No | | | | |
| AFV51369.1 | 5 | Streptomyces phage | 67 | No | Yes | No | | | | |
| | | phiCAM | | | | | | | | |
| AJG57936.1 | 6 | Bacillus cereus D17 | 49 | No | No | Yes | 396 | 727 | 1058 | 1389 |
| AKY03507.1 | 7 | Streptomyces phage | 19 | No | Yes | No | | | | |
| | | Danzina | | | | | | | | |
| AKY03881.1 | 8 | Streptomyces phage | 66 | No | Yes | No | | | | |
| | | Verse | | | | | | | | |
| AND10894.1 | 9 | Bacillus thuringiensis | 49 | No | No | Yes | 397 | 728 | 1059 | 1390 |
| | | serovar alesti | | | | | | | | |
| APC43293.1 | 10 | Streptomyces phage Joe | 19 | No | No | No | | | | |
| ASN71670.1 | 11 | Staphylococcus | 73 | No | No | Yes | 398 | 729 | 1090 | 1391 |
| | | epidermidis | | | | | | | | |
| BAA07372.1 | 12 | Streptomyces phage R4 | 67 | No | No | No | | | | |
| BAE05705.1 | 13 | Staphylococcus | 73 | No | No | No | | | | |
| | | haemolyticus | | | | | | | | |
| | | JCSC1435 | | | | | | | | |
| BAF03598.1 | 14 | Streptomyces phage | 13 | No | No | No | | | | |
| | | phiK38-l | | | | | | | | |
| BAF67264.1 | 15 | Staphylococcus aureus | 73 | No | No | No | | | | |
| | | subsp. aureus str. | | | | | | | | |
| | | Newman | | | | | | | | |
| BAG46462.1 | 16 | Burkholderia | 5 | No | No | No | | | | |
| | | multivorans ATCC | | | | | | | | |
| | | 17616 | | | | | | | | |
| CAD00410.1 | 17 | Bacteriophage A118] | 78 | No | No | No | | | | |
| | | [Listeria | | | | | | | | |
| | | monocytogenes EGD-e | | | | | | | | |
| CAR95427.1 | 18 | Streptococcus phage | 27 | No | No | No | | | | |
| | | phi-m46.1 | | | | | | | | |
| CBG73463.1 | 19 | Streptomyces scabiei | 41 | No | Yes | No | | | | |
| | | 87.22 | | | | | | | | |
| CYZ86932.1 | 20 | Streptococcus suis | 58 | Yes | No | Yes | 399 | 730 | 1061 | 1392 |
| EFD80439.2 | 21 | Fusobacterium | 82 | Yes | No | Yes | 400 | 731 | 1062 | 1393 |
| | | nucleatum subsp. | | | | | | | | |
| | | animalis D11 | | | | | | | | |
| EFR90504.1 | 22 | Listeria monocytogenes | 31 | Yes | No | Yes | 401 | 732 | 1063 | 1394 |
| EOE27531.1 | 23 | Enterococcus faecalis | 9 | Yes | No | Yes | 402 | 733 | 1064 | 1395 |
| | | EnGen0285 | | | | | | | | |
| EOK04340.1 | 24 | Enterococcus faecalis | 65 | No | No | Yes | 403 | 734 | 1065 | 1396 |
| | | EnGen0367 | | | | | | | | |
| EOP86000.1 | 25 | Bacillus cereus HuB4-4 | 53 | No | No | Yes | 404 | 735 | 1066 | 1397 |
| EQE33494.1 | 26 | Clostridioides difficile | 74 | No | Yes | Yes | 405 | 736 | 1067 | 1398 |
| ETI84184.1 | 27 | Streptococcus | 27 | No | No | Yes | 406 | 737 | 1068 | 1399 |
| | | anginosus DORA_7 | | | | | | | | |
| GDD80774.1 | 28 | Escherichia coli | 30 | Yes | Yes | Yes | 407 | 738 | 1069 | 1400 |
| KDF51021.1 | 29 | Enterobacter | 4 | Yes | Yes | Yes | 408 | 739 | 1070 | 1401 |
| | | roggenkampii CHS 79 | | | | | | | | |
| KEK15983.2 | 30 | Lactobacillus reuteri | 57 | No | No | Yes | 409 | 740 | 1071 | 1402 |
| KIS18008.1 | 31 | Streptococcus equi | 57 | No | No | Yes | 410 | 741 | 1072 | 1403 |
| | | subsp. zooepidemicus | | | | | | | | |
| | | Sz4is | | | | | | | | |
| KIS38487.1 | 32 | Stenotrophomonas | 5 | No | No | Yes | 411 | 742 | 1073 | 1404 |
| | | maltophilia WJ66 | | | | | | | | |
| KXO02427.1 | 33 | Bacillus thuringiensis | 49 | No | No | Yes | 412 | 743 | 1074 | 1405 |
| NP_047974.1 | 34 | Streptomyces virus | 2 | No | No | No | | | | |
| | | phiC31 | | | | | | | | |
| NP_112664.1 | 35 | Lactococcus phage | 54 | No | Yes | No | | | | |
| | | TP901-1 | | | | | | | | |
| NP_268897.1 | 36 | Streptococcus phage | 54 | No | No | No | | | | |
| | | 370.1 | | | | | | | | |
| NP_268897.1 | 37 | Streptococcus pyogenes | 54 | No | No | Yes | 413 | 744 | 1075 | 1406 |
| | | M1 GAS | | | | | | | | |
| NP_415076.1 | 38 | Escherichia coli str. K- | 42 | Yes | No | Yes | 414 | 745 | 1076 | 1407 |
| | | 12 substr. MG1655 | | | | | | | | |
| NP_463492.1 | 39 | Listeria monocytogenes | 78 | No | No | Yes | 415 | 746 | 1077 | 1408 |
| NP_470568.1 | 40 | Listeria innocua | 53 | No | No | No | | | | |
| | | Clip11262 | | | | | | | | |
| NP_813744.2 | 41 | Streptomyces virus | 7 | No | Yes | No | | | | |
| | | phiBT1 | | | | | | | | |
| NP_817623.1 | 42 | Mycobacterium virus | 32 | No | Yes | No | | | | |
| | | Bxz2 | | | | | | | | |
| NP_831691.1 | 43 | Bacillus cereus ATCC | 49 | No | No | Yes | 416 | 747 | 1078 | 1409 |
| | | 14579 | | | | | | | | |
| QBI96918.1 | 44 | Mycobacterium phage | 45 | No | No | No | | | | |
| | | Veracruz | | | | | | | | |
| SCC33377.1 | 45 | Bacillus cereus | 49 | No | No | Yes | 417 | 748 | 1079 | 1410 |
| SHX05262.1 | 46 | Mycobacteroides | 77 | Yes | Yes | Yes | 418 | 749 | 1080 | 1411 |
| | | abscessus subsp. | | | | | | | | |
| | | abscessus | | | | | | | | |
| SQB82501.1 | 47 | Streptococcus | 54 | No | No | Yes | 419 | 750 | 1081 | 1412 |
| | | dysgalactiae | | | | | | | | |
| SQI07626.1 | 48 | Streptococcus | 57 | No | Yes | Yes | 420 | 751 | 1082 | 1413 |
| | | pasteurianus | | | | | | | | |
| TBW91720.1 | 49 | Staphylococcus hominis | 73 | No | No | Yes | 421 | 752 | 1083 | 1414 |
| WP_000215775.1 | 50 | Bacillus cereus VD115 | 56 | No | No | Yes | 422 | 753 | 1084 | 1415 |
| WP_000286204.1 | 51 | Bacillus cereus MSX- | 35 | No | Yes | Yes | 423 | 754 | 1085 | 1416 |
| | | D12 | | | | | | | | |
| WP_000633501.1 | 52 | Streptococcus | 57 | No | No | Yes | 424 | 755 | 1086 | 1417 |
| | | agalactiae FSL S3-105 | | | | | | | | |
| WP_000633509.1 | 53 | Streptococcus | 57 | No | No | Yes | 425 | 756 | 1087 | 1418 |
| | | pneumoniae 670-6B | | | | | | | | |
| WP_000650392.1 | 54 | Bacillus thuringiensis | 70 | Yes | Yes | Yes | 426 | 757 | 1088 | 1419 |
| | | serovar kurstaki str. | | | | | | | | |
| | | YBT-1520 | | | | | | | | |
| WP_000709069.1 | 55 | Escherichia coli 5.0588 | 42 | Yes | No | Yes | 427 | 758 | 1089 | 1420 |
| WP_000709099.1 | 56 | Escherichia coli 55989 | 42 | Yes | No | Yes | 428 | 759 | 1090 | 1421 |
| WP_000844785.1 | 57 | Bacillus thuringiensis | 8 | No | No | Yes | 429 | 760 | 1091 | 1422 |
| | | serovar chinensis CT-43 | | | | | | | | |
| WP_000844788.1 | 58 | Bacillus thuringiensis | 8 | No | No | Yes | 430 | 761 | 1092 | 1423 |
| | | HD-789 | | | | | | | | |
| WP_000861306.1 | 59 | Staphylococcus aureus | 71 | No | No | Yes | 431 | 762 | 1093 | 1424 |
| | | subsp. aureus 132 | | | | | | | | |
| WP_000872533.1 | 60 | Bacillus sp. 2D03 | 49 | No | No | Yes | 432 | 763 | 1094 | 1425 |
| WP_000872535.1 | 61 | Bacillus cereus | 49 | No | No | Yes | 433 | 764 | 1095 | 1426 |
| | | BAG3X2-2 | | | | | | | | |
| WP_000989160.1 | 62 | Streptococcus | 57 | No | No | Yes | 434 | 765 | 1096 | 1427 |
| | | agalactiae FSL S3-277 | | | | | | | | |
| WP_001044789.1 | 63 | Streptococcus | 54 | No | No | Yes | 435 | 766 | 1097 | 1428 |
| | | agalactiae CCUG | | | | | | | | |
| | | 39096 A | | | | | | | | |
| WP_001233549.1 | 64 | Shigella boydii | 5 | No | No | Yes | 436 | 767 | 1098 | 1429 |
| WP_002165157.1 | 65 | Bacillus cereus VD048 | 8 | No | No | Yes | 437 | 768 | 1099 | 1430 |
| WP_002349497.1 | 66 | Enterococcus faecium | 9 | Yes | No | Yes | 438 | 769 | 1100 | 1431 |
| | | R501 | | | | | | | | |
| WP_002359484.1 | 67 | Enterococcus faecalis | 65 | No | No | Yes | 439 | 770 | 1101 | 1432 |
| WP_002381434.1 | 68 | Enterococcus faecalis | 65 | No | No | Yes | 440 | 771 | 1102 | 1433 |
| WP_002399935.1 | 69 | Enterococcus faecalis | 65 | No | No | Yes | 441 | 772 | 1103 | 1434 |
| | | TX0309B | | | | | | | | |
| WP_002409538.1 | 70 | Enterococcus faecalis | 65 | No | No | Yes | 442 | 773 | 1104 | 1435 |
| | | TX0645 | | | | | | | | |
| WP_002416055.1 | 71 | Enterococcus faecalis | 65 | No | No | Yes | 443 | 774 | 1105 | 1436 |
| | | ERV103 | | | | | | | | |
| WP_002469492.1 | 72 | Staphylococcus | 73 | No | No | Yes | 444 | 775 | 1106 | 1437 |
| | | epidermidis | | | | | | | | |
| WP_002475509.1 | 73 | Staphylococcus | 73 | No | No | Yes | 445 | 776 | 1107 | 1438 |
| | | epidermidis 14.1.R1.SE | | | | | | | | |
| WP_002502891.1 | 74 | Staphylococcus | 73 | No | No | Yes | 446 | 777 | 1108 | 1439 |
| | | epidermidis NIHLM003 | | | | | | | | |
| WP_003199542.1 | 75 | Bacillus | 8 | No | No | Yes | 447 | 778 | 1109 | 1440 |
| | | pseudomycoides | | | | | | | | |
| WP_003365993.1 | 76 | Clostridium botulinum | 40 | Yes | Yes | Yes | 448 | 779 | 1110 | 1441 |
| | | C str. Eklund | | | | | | | | |
| WP_003514343.1 | 77 | Hungateiclostridium | 82 | Yes | Yes | Yes ^{T} | 449 | 780 | 1111 | 1442 |
| | | thermocellum JW20 | | | | | | | | |
| WP_003727736.1 | 78 | Listeria monocytogenes | 78 | No | No | Yes | 450 | 781 | 1112 | 1443 |
| | | J0161 | | | | | | | | |
| WP_003731148.1 | 79 | Listeria monocytogenes | 31 | Yes | No | Yes | 451 | 782 | 1113 | 1444 |
| | | FSL N1-017 | | | | | | | | |
| WP_003731150.1 | 80 | Listeria monocytogenes | 27 | No | No | Yes | 452 | 783 | 1114 | 1445 |
| WP_003770016.1 | 81 | Listeria innocua | 78 | No | No | Yes | 453 | 784 | 1115 | 1446 |
| WP_003903979.1 | 82 | Mycobacterium | 69 | No | Yes | No | | | | |
| | | tuberculosis | | | | | | | | |
| WP_005908927.1 | 83 | Fusobacterium | 63 | Yes | No | Yes | 454 | 785 | 1116 | 1447 |
| | | nucleatum subsp. | | | | | | | | |
| | | animalis F0419 | | | | | | | | |
| WP_008698549.1 | 84 | Fusobacterium | 61 | Yes | Yes | Yes | 455 | 786 | 1117 | 1448 |
| | | ulcerans 12-1B | | | | | | | | |
| WP_008700773.1 | 85 | Fusobacterium | 63 | Yes | Yes | Yes | 456 | 787 | 1118 | 1449 |
| | | nucleatum subsp. | | | | | | | | |
| | | polymorphum F0401 | | | | | | | | |
| WP_009269238.1 | 86 | Enterococcus faecium | 9 | Yes | No | Yes | 457 | 788 | 1119 | 1450 |
| WP_009269239.1 | 87 | Enterococcus faecium | 9 | Yes | Yes | Yes | 458 | 789 | 1120 | 1451 |
| WP_009329281.1 | 88 | Bacillus licheniformis | 59 | No | No | Yes | 459 | 790 | 1121 | 1452 |
| WP_010082246.1 | 89 | Wolbachia | 52 | Yes | Yes | Yes | 460 | 791 | 1122 | 1453 |
| | | endosymbiont of | | | | | | | | |
| | | Drosophila simulans wAu | | | | | | | | |
| WP_010708035.1 | 90 | Enterococcus faecalis | 65 | No | No | Yes | 461 | 792 | 1123 | 1454 |
| | | EnGen0061 | | | | | | | | |
| WP_010717149.1 | 91 | Enterococcus faecalis | 65 | No | Yes | Yes | 462 | 793 | 1124 | 1455 |
| | | EnGen0115 | | | | | | | | |
| WP_010725837.1 | 92 | Enterococcus faecium | 80 | Yes | Yes | Yes | 463 | 794 | 1125 | 1456 |
| | | EnGen0163 | | | | | | | | |
| WP_010826647.1 | 93 | Enterococcus faecalis | 65 | No | No | Yes | 464 | 795 | 1126 | 1457 |
| | | EnGen0359 | | | | | | | | |
| WP_010990844.1 | 94 | Listeria innocua | 53 | No | No | Yes | 465 | 796 | 1127 | 1458 |
| | | Clip11262 | | | | | | | | |
| WP_010991183.1 | 95 | Listeria innocua | 78 | No | No | Yes | 466 | 797 | 1128 | 1459 |
| | | Clip11262 | | | | | | | | |
| WP_011017563.1 | 96 | Streptococcus pyogenes | 54 | No | No | Yes | 467 | 798 | 1129 | 1460 |
| | | MGAS10270 | | | | | | | | |
| WP_011276651.1 | 97 | Staphylococcus | 73 | No | No | Yes | 468 | 799 | 1130 | 1461 |
| | | haemolyticus | | | | | | | | |
| | | JCSC1435 | | | | | | | | |
| WP_012991015.1 | 98 | Staphylococcus | 73 | No | No | Yes | 469 | 800 | 1131 | 1462 |
| | | lugdunensis HKU09-01 | | | | | | | | |
| WP_013237059.1 | 99 | Clostridium ljungdahlii | 27 | No | Yes | Yes | 470 | 801 | 1132 | 1463 |
| | | DSM 13528 | | | | | | | | |
| WP_013524454.1 | 100 | Geobacillus sp. | 56 | No | No | Yes | 471 | 802 | 1133 | 1464 |
| | | Y412MC61 | | | | | | | | |
| WP_014387031.1 | 101 | Enterococcus faecium | 27 | No | No | Yes | 472 | 803 | 1134 | 1465 |
| | | Aus0004 | | | | | | | | |
| WP_014636355.1 | 102 | Streptococcus suis | 84 | Yes | No | Yes | 473 | 804 | 1135 | 1466 |
| WP_014929968.1 | 103 | Listeria monocytogenes | 27 | No | No | Yes | 474 | 805 | 1136 | 1467 |
| | | FSL N1-017 | | | | | | | | |
| WP_014930216.1 | 104 | Listeria monocytogenes | 78 | No | No | No | | | | |
| WP_015407429.1 | 105 | Dehalococcoides | 51 | Yes | Yes | Yes | 475 | 806 | 1137 | 1468 |
| | | mccartyi BTF08 | | | | | | | | |
| WP_015407430.1 | 106 | Dehalococcoides | 9 | Yes | No | Yes | 476 | 807 | 1138 | 1469 |
| | | mccartyi BTF08 | | | | | | | | |
| WP_015407431.1 | 107 | Dehalococcoides | 83 | Yes | Yes | Yes | 477 | 808 | 1139 | 1470 |
| | | mccartyi BTF08 | | | | | | | | |
| WP_015611741.1 | 108 | Streptomyces | 17 | No | No | Yes | 478 | 809 | 1140 | 1471 |
| | | fulvissimus DSM 40593 | | | | | | | | |
| WP_015891191.1 | 109 | Brevibacillus brevis | 57 | No | No | Yes | 479 | 810 | 1141 | 1472 |
| | | NBRC 100599 | | | | | | | | |
| WP_015957900.1 | 110 | Clostridium botulinum | 8 | No | No | Yes | 480 | 811 | 1142 | 1473 |
| | | B1 str. Okra | | | | | | | | |
| WP_016097900.1 | 111 | Bacillus cereus HuB4-4 | 70 | Yes | No | Yes | 481 | 812 | 1143 | 1474 |
| WP_016130176.1 | 112 | Bacillus cereus | 8 | No | No | Yes | 482 | 813 | 1144 | 1475 |
| | | VDM053 | | | | | | | | |
| WP_016570474.1 | 113 | Streptomyces albulus | 29 | Yes | Yes | Yes | 483 | 814 | 1145 | 1476 |
| | | ZPM | | | | | | | | |
| WP_017696931.1 | 114 | Bacillus subtilis S1-4 | 36 | No | No | Yes | 484 | 815 | 1146 | 1477 |
| WP_019725860.1 | 115 | Pseudomonas | 5 | No | No | Yes | 485 | 816 | 1147 | 1478 |
| | | aeruginosa 213BR | | | | | | | | |
| WP_021374870.1 | 116 | Clostridioides difficile | 8 | No | No | Yes | 486 | 817 | 1148 | 1479 |
| WP_021534391.1 | 117 | Escherichia coli HVH | 30 | Yes | No | Yes | 487 | 818 | 1149 | 1480 |
| | | 147 (4-5893887) | | | | | | | | |
| WP_021775307.1 | 118 | Streptococcus pyogenes | 54 | No | No | Yes | 488 | 819 | 1150 | 1481 |
| | | GA41046 | | | | | | | | |
| WP_023107160.1 | 119 | Pseudomonas | 5 | No | No | Yes | 489 | 820 | 1151 | 1482 |
| | | aeruginosa BL04 | | | | | | | | |
| WP_023115516.1 | 120 | Pseudomonas | 5 | No | No | Yes | 490 | 821 | 1152 | 1483 |
| | | aeruginosa | | | | | | | | |
| | | BWHPSA021 | | | | | | | | |
| WP_023552493.1 | 121 | Listeria monocytogenes | 78 | No | No | Yes | 491 | 822 | 1153 | 1484 |
| WP_024052970.1 | 122 | Streptococcus sp. | 84 | Yes | Yes | Yes | 492 | 823 | 1154 | 1485 |
| | | HMSC034E12 | | | | | | | | |
| WP_024233971.1 | 123 | Escherichia coli STEC | 14 | Yes | Yes | Yes | 493 | 824 | 1155 | 1486 |
| | | O174:H46 str. 1-151 | | | | | | | | |
| WP_024399342.1 | 124 | Streptococcus suis 89- | 84 | Yes | No | Yes | 494 | 825 | 1156 | 1487 |
| | | 5259 | | | | | | | | |
| WP_025191276.1 | 125 | Enterococcus faecalis | 65 | No | No | Yes | 495 | 826 | 1157 | 1488 |
| | | EnGen0367 | | | | | | | | |
| WP_025782674.1 | 126 | Clostridioides difficile | 74 | No | No | Yes | 496 | 827 | 1158 | 1489 |
| | | CD211 | | | | | | | | |
| WP_028992649.1 | 127 | Thermoanaerobacter | 31 | Yes | Yes | Yes ^{T} | 497 | 828 | 1159 | 1490 |
| | | thermocopriae JCM | | | | | | | | |
| | | 7501 | | | | | | | | |
| WP_029159931.1 | 128 | Clostridium | 18 | Yes | Yes | Yes | 498 | 829 | 1160 | 1491 |
| | | scatologenes | | | | | | | | |
| WP_031642347.1 | 129 | Listeria monocytogenes | 78 | No | No | Yes | 499 | 830 | 1161 | 1492 |
| WP_031645248.1 | 130 | Listeria monocytogenes | 78 | No | No | Yes | 500 | 831 | 1162 | 1493 |
| WP_031645680.1 | 131 | Listeria monocytogenes | 78 | No | No | Yes | 501 | 832 | 1163 | 1494 |
| WP_031673611.1 | 132 | Pseudomonas | 5 | No | No | Yes | 502 | 833 | 1164 | 1495 |
| | | aeruginosa | | | | | | | | |
| WP_031788255.1 | 133 | Staphylococcus aureus | 71 | No | No | Yes | 503 | 834 | 1165 | 1496 |
| WP_031890776.1 | 134 | Staphylococcus aureus | 71 | No | No | Yes | 504 | 835 | 1166 | 1497 |
| WP_033654380.1 | 135 | Enterococcus faecium | 27 | No | No | Yes | 505 | 836 | 1167 | 1498 |
| | | R501 | | | | | | | | |
| WP_033943750.1 | 136 | Pseudomonas | 5 | No | No | Yes | 506 | 837 | 1168 | 1499 |
| | | aeruginosa | | | | | | | | |
| WP_035338239.1 | 137 | Bacillus | 59 | No | No | Yes | 507 | 838 | 1169 | 1500 |
| | | paralicheniformis | | | | | | | | |
| WP_035437377.1 | 138 | Lactobacillus | 15 | Yes | Yes | Yes | 508 | 839 | 1170 | 1501 |
| | | fermentum | | | | | | | | |
| WP_035437379.1 | 139 | Lactobacillus | 9 | Yes | No | Yes | 509 | 840 | 1171 | 1502 |
| | | fermentum | | | | | | | | |
| WP_037835118.1 | 140 | Streptomyces sp. NRRL | 25 | Yes | Yes | Yes | 510 | 841 | 1172 | 1503 |
| | | S-455 | | | | | | | | |
| WP_038521242.1 | 141 | Streptomyces albulus | 29 | Yes | No | Yes | 511 | 842 | 1173 | 1504 |
| WP_039388693.1 | 142 | Listeria monocytogenes | 78 | No | No | Yes | 512 | 843 | 1174 | 1505 |
| WP_039660878.1 | 143 | Pantoea sp. MBLJ3 | 46 | Yes | Yes | Yes | 513 | 844 | 1175 | 1506 |
| WP_042515162.1 | 144 | Bacillus cereus | 49 | No | No | Yes | 514 | 845 | 1176 | 1507 |
| WP_043503403.1 | 145 | Pseudomonas | 5 | No | No | Yes | 515 | 846 | 1177 | 1508 |
| | | aeruginosa | | | | | | | | |
| WP_044751504.1 | 146 | Xanthomonas oryzae | 5 | No | Yes | Yes | 516 | 847 | 1178 | 1509 |
| | | pv. oryzicola | | | | | | | | |
| WP_044791785.1 | 147 | Bacillus thuringiensis | 76 | Yes | Yes | Yes | 517 | 848 | 1179 | 1510 |
| WP_044981554.1 | 148 | Streptococcus suis | 58 | Yes | Yes | Yes | 518 | 849 | 1180 | 1511 |
| WP_045667426.1 | 149 | Geobacter | 75 | Yes | No | Yes | 519 | 850 | 1181 | 1512 |
| | | sulfurreducens | | | | | | | | |
| WP_046058042.1 | 150 | Clostridioides difficile | 31 | Yes | No | Yes | 520 | 851 | 1182 | 1513 |
| WP_046377505.1 | 151 | Listeria monocytogenes | 78 | No | No | Yes | 521 | 852 | 1183 | 1514 |
| WP_046559965.1 | 152 | Bacillus velezensis | 59 | No | No | Yes | 522 | 853 | 1184 | 1515 |
| WP_046655502.1 | 153 | Clostridium tetani | 8 | No | No | Yes | 523 | 854 | 1185 | 1516 |
| WP_046811198.1 | 154 | Listeria monocytogenes | 64 | Yes | Yes | Yes | 524 | 855 | 1186 | 1517 |
| WP_048020573.1 | 155 | Bacillus aryabhattai | 53 | No | No | Yes | 525 | 856 | 1187 | 1518 |
| WP_048962262.1 | 156 | Enterococcus faecalis | 65 | No | No | Yes | 526 | 857 | 1188 | 1519 |
| WP_049368564.1 | 157 | Staphylococcus | 73 | No | No | Yes | 527 | 858 | 1189 | 1520 |
| | | epidermidis | | | | | | | | |
| WP_049381135.1 | 158 | Staphylococcus | 71 | No | No | Yes | 528 | 859 | 1190 | 1521 |
| | | epidermidis | | | | | | | | |
| WP_049401331.1 | 159 | Staphylococcus | 73 | No | No | Yes | 529 | 860 | 1191 | 1522 |
| | | epidermidis | | | | | | | | |
| WP_049431410.1 | 160 | Staphylococcus hominis | 73 | No | No | Yes | 530 | 861 | 1192 | 1523 |
| WP_049492617.1 | 161 | Streptococcus | 57 | No | No | Yes | 531 | 862 | 1193 | 1524 |
| | | pseudopneumoniae | | | | | | | | |
| WP_049891860.1 | 162 | Listeria monocytogenes | 78 | No | No | Yes | 532 | 863 | 1194 | 1525 |
| WP_050330935.1 | 163 | Staphylococcus | 71 | No | No | Yes | 533 | 864 | 1195 | 1526 |
| | | schleiferi | | | | | | | | |
| WP_050337544.1 | 164 | Staphylococcus | 71 | No | No | Yes | 534 | 865 | 1196 | 1527 |
| | | schleiferi | | | | | | | | |
| WP_051428004.1 | 165 | Paenibacillus larvae | 86 | Yes | Yes | Yes | 535 | 866 | 1197 | 1528 |
| | | subsp. larvae DSM | | | | | | | | |
| | | 25719 | | | | | | | | |
| WP_051626736.1 | 166 | Caballeronia | 6 | Yes | Yes | Yes | 536 | 867 | 1198 | 1529 |
| | | jiangsuensis | | | | | | | | |
| WP_052263176.1 | 167 | Clostridium | 40 | Yes | No | Yes | 537 | 868 | 1199 | 1530 |
| | | tyrobutyricum | | | | | | | | |
| WP_052497231.1 | 168 | Bacillus thuringiensis | 62 | No | No | Yes | 538 | 869 | 1200 | 1531 |
| | | serovar morrisoni | | | | | | | | |
| WP_052506912.1 | 169 | Streptococcus suis | 88 | Yes | Yes | Yes | 539 | 870 | 1201 | 1532 |
| WP_053020692.1 | 170 | Staphylococcus | 72 | Yes | No | Yes | 540 | 871 | 1202 | 1533 |
| | | haemolyticus | | | | | | | | |
| WP_053028958.1 | 171 | Staphylococcus | 73 | No | Yes | Yes | 541 | 872 | 1203 | 1534 |
| | | haemolyticus | | | | | | | | |
| WP_053290296.1 | 172 | Clostridium botulinum | 40 | Yes | No | Yes | 542 | 873 | 1204 | 1535 |
| WP_053497239.1 | 173 | Stenotrophomonas | 5 | No | No | Yes | 543 | 874 | 1205 | 1536 |
| | | maltophilia | | | | | | | | |
| WP_053512967.1 | 174 | Bacillus thuringiensis | 76 | Yes | No | Yes | 544 | 875 | 1206 | 1537 |
| | | serovar andalousiensis | | | | | | | | |
| WP_053903616.1 | 175 | Escherichia coli | 20 | Yes | Yes | Yes | 545 | 876 | 1207 | 1538 |
| WP_057383473.1 | 176 | Pseudomonas | 5 | No | No | Yes | 546 | 877 | 1208 | 1539 |
| | | aeruginosa | | | | | | | | |
| WP_057385580.1 | 177 | Pseudomonas | 5 | No | No | Yes | 547 | 878 | 1209 | 1540 |
| | | aeruginosa | | | | | | | | |
| WP_058016331.1 | 178 | Pseudomonas | 5 | No | No | Yes | 548 | 879 | 1210 | 1541 |
| | | aeruginosa | | | | | | | | |
| WP_058085641.1 | 179 | Clostridioides difficile | 27 | No | No | Yes | 549 | 880 | 1211 | 1542 |
| WP_058831750.1 | 180 | Listeria monocytogenes | 53 | No | No | Yes | 550 | 881 | 1212 | 1543 |
| WP_059456121.1 | 181 | Burkholderia | 5 | No | No | Yes | 551 | 882 | 1213 | 1544 |
| | | vietnamiensis | | | | | | | | |
| WP_059460907.1 | 182 | Burkholderia | 5 | No | No | Yes | 552 | 883 | 1214 | 1545 |
| | | vietnamiensis | | | | | | | | |
| WP_060670310.1 | 183 | Clostridium perfringens | 44 | Yes | Yes | Yes | 553 | 884 | 1215 | 1546 |
| WP_060798679.1 | 184 | Fusobacterium | 63 | Yes | No | Yes | 554 | 885 | 1216 | 1547 |
| | | nucleatum | | | | | | | | |
| WP_060868949.1 | 185 | Listeria monocytogenes | 31 | Yes | No | Yes | 555 | 886 | 1217 | 1548 |
| WP_061114351.1 | 186 | Listeria monocytogenes | 31 | Yes | No | Yes | 556 | 887 | 1218 | 1549 |
| WP_061322114.1 | 187 | Clostridium botulinum | 31 | Yes | No | Yes | 557 | 888 | 1219 | 1550 |
| WP_061355600.1 | 188 | Escherichia coli | 30 | Yes | No | Yes | 558 | 889 | 1220 | 1551 |
| WP_061660420.1 | 189 | Bacillus cereus | 68 | Yes | No | Yes | 559 | 890 | 1221 | 1552 |
| WP_061664507.1 | 190 | Listeria monocytogenes | 78 | No | No | Yes | 560 | 891 | 1222 | 1553 |
| WP_062078525.1 | 191 | Staphylococcus sp. | 73 | No | No | Yes | 561 | 892 | 1223 | 1554 |
| | | HMSC062D12 | | | | | | | | |
| WP_062723120.1 | 192 | Streptomyces | 17 | No | Yes | Yes | 562 | 893 | 1224 | 1555 |
| | | caeruleatus | | | | | | | | |
| WP_063280150.1 | 193 | Staphylococcus | 73 | No | No | Yes | 563 | 894 | 1225 | 1556 |
| | | epidermidis | | | | | | | | |
| WP_063855923.1 | 194 | Enterococcus faecalis | 79 | Yes | No | Yes | 564 | 895 | 1226 | 1557 |
| WP_064034122.1 | 195 | Listeria monocytogenes | 31 | Yes | No | Yes | 565 | 896 | 1227 | 1558 |
| WP_064206928.1 | 196 | Staphylococcus hominis | 73 | No | No | Yes | 566 | 897 | 1228 | 1559 |
| WP_064297673.1 | 197 | Ralstonia | 5 | No | No | Yes | 567 | 898 | 1229 | 1560 |
| | | solanacearum | | | | | | | | |
| WP_064470310.1 | 198 | Bacillus wiedmannii | 8 | No | No | Yes | 568 | 899 | 1230 | 1561 |
| WP_064549840.1 | 199 | Parageobacillus | 56 | No | Yes | Yes ^{T} | 569 | 900 | 1231 | 1562 |
| | | thermoglucosidasius | | | | | | | | |
| WP_064963684.1 | 200 | Paenibacillus polymyxa | 43 | Yes | Yes | Yes | 570 | 901 | 1232 | 1563 |
| WP_065354608.1 | 201 | Staphylococcus | 73 | No | No | Yes | 571 | 902 | 1233 | 1564 |
| | | pseudintermedius | | | | | | | | |
| WP_065724346.1 | 202 | Stenotrophomonas | 5 | No | No | Yes | 572 | 903 | 1234 | 1565 |
| | | maltophilia | | | | | | | | |
| WP_065733410.1 | 203 | Streptococcus | 54 | No | No | Yes | 573 | 904 | 1235 | 1566 |
| | | agalactiae | | | | | | | | |
| WP_066028610.1 | 204 | Streptococcus | 54 | No | No | Yes | 574 | 905 | 1236 | 1567 |
| | | dysgalactiae subsp. | | | | | | | | |
| | | equisimilis | | | | | | | | |
| WP_066864475.1 | 205 | Sphingobium sp. TCM1 | 26 | Yes | Yes | Yes | 575 | 906 | 1237 | 1568 |
| WP_069002610.1 | 206 | Listeria monocytogenes | 78 | No | No | Yes | 576 | 907 | 1238 | 1569 |
| WP_069019758.1 | 207 | Listeria monocytogenes | 64 | Yes | No | Yes | 577 | 908 | 1239 | 1570 |
| WP_069482207.1 | 208 | Lysinibacillus | 59 | No | Yes | Yes | 578 | 909 | 1240 | 1571 |
| | | fusiformis | | | | | | | | |
| WP_069500683.1 | 209 | Bacillus licheniformis | 59 | No | No | Yes | 579 | 910 | 1241 | 1572 |
| WP_070021558.1 | 210 | Staphylococcus aureus | 73 | No | No | Yes | 580 | 911 | 1242 | 1573 |
| WP_070030387.1 | 211 | Listeria monocytogenes | 78 | No | No | Yes | 581 | 912 | 1243 | 1574 |
| WP_070080197.1 | 212 | Escherichia coli | 42 | Yes | Yes | Yes | 582 | 913 | 1244 | 1575 |
| | | O157:H7 | | | | | | | | |
| WP_070210520.1 | 213 | Listeria monocytogenes | 31 | Yes | No | Yes | 583 | 914 | 1245 | 1576 |
| WP_070210526.1 | 214 | Listeria monocytogenes | 27 | No | No | Yes | 584 | 915 | 1246 | 1577 |
| WP_070254894.1 | 215 | Listeria monocytogenes | 78 | No | Yes | Yes | 585 | 916 | 1247 | 1578 |
| WP_070481549.1 | 216 | Staphylococcus sp. | 71 | No | No | Yes | 586 | 917 | 1248 | 1579 |
| | | HMSC068D08 | | | | | | | | |
| WP_070597291.1 | 217 | Staphylococcus sp. | 71 | No | Yes | Yes | 587 | 918 | 1249 | 1580 |
| | | HMSC068C09 | | | | | | | | |
| WP_070780189.1 | 218 | Clostridium sp. | 23 | Yes | No | Yes | 588 | 919 | 1250 | 1581 |
| | | HMSC19A10 | | | | | | | | |
| WP_070781449.1 | 219 | Listeria monocytogenes | 78 | No | No | Yes | 589 | 920 | 1251 | 1582 |
| WP_070784918.1 | 220 | Listeria monocytogenes | 78 | No | No | Yes | 590 | 921 | 1252 | 1583 |
| WP_070858703.1 | 221 | Staphylococcus sp. | 73 | No | No | Yes | 591 | 922 | 1253 | 1584 |
| | | HMSC077D09 | | | | | | | | |
| WP_071218019.1 | 222 | Paenibacillus sp. | 39 | Yes | Yes | Yes | 592 | 923 | 1254 | 1585 |
| | | LC231 | | | | | | | | |
| WP_071647453.1 | 223 | Clostridium botulinum | 8 | No | No | Yes | 593 | 924 | 1255 | 1586 |
| WP_071661745.1 | 224 | Listeria monocytogenes | 78 | No | No | Yes | 594 | 925 | 1256 | 1587 |
| WP_072217376.1 | 225 | Listeria monocytogenes | 78 | No | No | Yes | 595 | 926 | 1257 | 1588 |
| WP_073206676.1 | 226 | Bacillus safensis | 53 | No | No | Yes | 596 | 927 | 1258 | 1589 |
| WP_073656028.1 | 227 | Pseudomonas | 52 | Yes | No | Yes | 597 | 928 | 1259 | 1590 |
| | | aeruginosa | | | | | | | | |
| WP_073656076.1 | 228 | Pseudomonas | 16 | Yes | No | Yes | 598 | 929 | 1260 | 1591 |
| | | aeruginosa | | | | | | | | |
| WP_074046931.1 | 229 | Listeria monocytogenes | 78 | No | No | Yes | 599 | 930 | 1261 | 1592 |
| WP_074196983.1 | 230 | Pseudomonas | 5 | No | No | Yes | 600 | 931 | 1262 | 1593 |
| | | aeruginosa | | | | | | | | |
| WP_075841482.1 | 231 | Clostridium perfringens | 44 | Yes | No | Yes | 601 | 932 | 1263 | 1594 |
| WP_076231728.1 | 232 | Clostridium botulinum | 18 | Yes | No | Yes | 602 | 933 | 1264 | 1595 |
| | | B2 128 | | | | | | | | |
| WP_076613438.1 | 233 | Clostridioides difficile | 8 | No | No | Yes | 603 | 934 | 1265 | 1596 |
| WP_076934419.1 | 234 | Burkholderia | 75 | Yes | Yes | Yes | 604 | 935 | 1266 | 1597 |
| | | pseudomallei | | | | | | | | |
| WP_077143729.1 | 235 | Enterococcus faecalis | 65 | No | No | Yes | 605 | 936 | 1267 | 1598 |
| WP_077319577.1 | 236 | Listeria monocytogenes | 31 | Yes | No | Yes | 606 | 937 | 1268 | 1599 |
| WP_077700294.1 | 237 | Staphylococcus hominis | 73 | No | No | Yes | 607 | 938 | 1269 | 1600 |
| WP_078177817.1 | 238 | Bacillus mycoides | 8 | No | No | Yes | 608 | 939 | 1270 | 1601 |
| WP_078209883.1 | 239 | Clostridium perfringens | 50 | Yes | Yes | Yes | 609 | 940 | 1271 | 1602 |
| WP_079167461.1 | 240 | Streptomyces | 13 | No | Yes | Yes | 610 | 941 | 1272 | 1603 |
| | | nanshensis | | | | | | | | |
| WP_079253086.1 | 241 | Streptococcus suis | 27 | No | No | Yes | 611 | 942 | 1273 | 1604 |
| WP_079270014.1 | 242 | Streptococcus suis 89- | 27 | No | No | Yes | 612 | 943 | 1274 | 1605 |
| | | 5259 | | | | | | | | |
| WP_079448828.1 | 243 | Listeria monocytogenes | 78 | No | No | Yes | 613 | 944 | 1275 | 1606 |
| WP_079757549.1 | 244 | Streptococcus sp. | 27 | No | No | Yes | 614 | 945 | 1276 | 1607 |
| | | HMSC034E12 | | | | | | | | |
| WP_080118482.1 | 245 | Bacillus cereus HuB4-4 | 53 | No | Yes | Yes | 615 | 946 | 1277 | 1608 |
| WP_080141533.1 | 246 | Listeria monocytogenes | 78 | No | No | Yes | 616 | 947 | 1278 | 1609 |
| WP_080334512.1 | 247 | Bacillus cereus D17 | 49 | No | No | Yes | 617 | 948 | 1279 | 1610 |
| WP_080499134.1 | 248 | Burkholderia | 16 | Yes | Yes | Yes | 618 | 949 | 1280 | 1611 |
| | | pseudomallei | | | | | | | | |
| WP_080624080.1 | 249 | Bacillus licheniformis | 38 | Yes | Yes | Yes | 619 | 950 | 1281 | 1612 |
| WP_080626969.1 | 250 | Bacillus licheniformis | 59 | No | No | Yes | 620 | 951 | 1282 | 1613 |
| WP_081101985.1 | 251 | Bacillus thuringiensis | 49 | No | No | Yes | 621 | 952 | 1283 | 1614 |
| WP_081113934.1 | 252 | Bacillus thuringiensis | 49 | No | No | Yes | 622 | 953 | 1284 | 1615 |
| WP_081115824.1 | 253 | Enterococcus faecalis | 79 | Yes | No | Yes | 623 | 954 | 1285 | 1616 |
| WP_081225183.1 | 254 | Staphylococcus xylosus | 72 | Yes | Yes | Yes | 624 | 955 | 1286 | 1617 |
| WP_081252865.1 | 255 | Bacillus thuringiensis | 49 | No | No | Yes | 625 | 956 | 1287 | 1618 |
| | | serovar alesti | | | | | | | | |
| WP_082870750.1 | 256 | Nocardia terpenica | 3 | Yes | Yes | Yes | 626 | 957 | 1288 | 1619 |
| WP_083983188.1 | 257 | Streptococcus | 54 | No | No | Yes | 627 | 958 | 1289 | 1620 |
| | | pneumoniae | | | | | | | | |
| WP_084882551.1 | 258 | Streptococcus oralis | 57 | No | No | Yes | 628 | 959 | 1290 | 1621 |
| | | subsp. oralis | | | | | | | | |
| WP_085060457.1 | 259 | Staphylococcus | 73 | No | No | Yes | 629 | 960 | 1291 | 1622 |
| | | haemolyticus | | | | | | | | |
| WP_085317587.1 | 260 | Staphylococcus | 73 | No | No | Yes | 630 | 961 | 1292 | 1623 |
| | | lugdunensis | | | | | | | | |
| WP_085430121.1 | 261 | Sporosarcina sp. P37 | 59 | No | No | Yes | 631 | 962 | 1293 | 1624 |
| WP_085547454.1 | 262 | Burkholderia | 75 | Yes | No | Yes | 632 | 963 | 1294 | 1625 |
| | | pseudomallei | | | | | | | | |
| WP_085547864.1 | 263 | Burkholderia | 16 | Yes | No | Yes | 633 | 964 | 1295 | 1626 |
| | | pseudomallei | | | | | | | | |
| WP_085707778.1 | 264 | Listeria monocytogenes | 78 | No | No | Yes | 634 | 965 | 1296 | 1627 |
| WP_087994267.1 | 265 | Bacillus thuringiensis | 78 | No | No | Yes | 635 | 966 | 1297 | 1628 |
| | | serovar konkukian | | | | | | | | |
| WP_088034496.1 | 266 | Bacillus thuringiensis | 8 | No | No | Yes | 636 | 967 | 1298 | 1629 |
| | | serovar navarrensis | | | | | | | | |
| WP_088113025.1 | 267 | Bacillus cereus | 49 | No | Yes | Yes | 637 | 968 | 1299 | 1630 |
| WP_089602000.1 | 268 | Salmonella enterica | 34 | Yes | Yes | Yes | 638 | 969 | 1300 | 1631 |
| WP_089997567.1 | 269 | Leuconostoc gelidum | 54 | No | No | Yes | 639 | 970 | 1301 | 1632 |
| | | subsp. gasicomitatum | | | | | | | | |
| WP_090835057.1 | 270 | Bacillus sp. ok634 | 56 | No | No | Yes | 640 | 971 | 1302 | 1633 |
| WP_094146498.1 | 271 | Shigella sonnei | 87 | Yes | Yes | Yes | 641 | 972 | 1303 | 1634 |
| WP_094396560.1 | 272 | Bacillus cytotoxicus | 62 | No | Yes | Yes | 642 | 973 | 1304 | 1635 |
| WP_096541455.1 | 273 | Enterococcus faecium | 31 | Yes | No | Yes | 643 | 974 | 1305 | 1636 |
| WP_096541458.1 | 274 | Enterococcus faecium | 27 | No | No | Yes | 644 | 975 | 1306 | 1637 |
| WP_096812886.1 | 275 | Listeria monocytogenes | 27 | No | No | Yes | 645 | 976 | 1307 | 1638 |
| WP_096865359.1 | 276 | Listeria monocytogenes | 78 | No | No | Yes | 646 | 977 | 1308 | 1639 |
| WP_096874316.1 | 277 | Listeria monocytogenes | 78 | No | No | Yes | 647 | 978 | 1309 | 1640 |
| WP_096962681.1 | 278 | Escherichia coli | 30 | Yes | No | Yes | 648 | 979 | 1310 | 1641 |
| WP_097501458.1 | 279 | Listeria monocytogenes | 27 | No | No | Yes | 649 | 980 | 1311 | 1642 |
| WP_097517744.1 | 280 | Listeria monocytogenes | 78 | No | No | Yes | 650 | 981 | 1312 | 1643 |
| WP_097528742.1 | 281 | Listeria innocua | 78 | No | No | Yes | 651 | 982 | 1313 | 1644 |
| WP_097529020.1 | 282 | Listeria monocytogenes | 78 | No | No | Yes | 652 | 983 | 1314 | 1645 |
| WP_097807826.1 | 283 | Bacillus thuringiensis | 68 | Yes | No | Yes | 653 | 984 | 1315 | 1646 |
| WP_097877701.1 | 284 | Bacillus cereus | 49 | No | No | Yes | 654 | 985 | 1316 | 1647 |
| WP_097988599.1 | 285 | Bacillus | 8 | No | No | Yes | 655 | 986 | 1317 | 1648 |
| | | pseudomycoides | | | | | | | | |
| WP_098035084.1 | 286 | Lactobacillus sp. | 57 | No | No | Yes | 656 | 987 | 1318 | 1649 |
| | | UMNPBX13 | | | | | | | | |
| WP_098046740.1 | 287 | Lactobacillus sp. | 57 | No | No | Yes | 657 | 988 | 1319 | 1650 |
| | | UMNPBX10 | | | | | | | | |
| WP_098091951.1 | 288 | Bacillus wiedmannii | 8 | No | No | Yes | 658 | 989 | 1320 | 1651 |
| WP_098161179.1 | 289 | Bacillus | 8 | No | No | Yes | 659 | 990 | 1321 | 1652 |
| | | pseudomycoides | | | | | | | | |
| WP_098188118.1 | 290 | Bacillus | 8 | No | No | Yes | 660 | 991 | 1322 | 1653 |
| | | pseudomycoides | | | | | | | | |
| WP_098360688.1 | 291 | Bacillus thuringiensis | 68 | Yes | No | Yes | 661 | 992 | 1323 | 1654 |
| WP_098367614.1 | 292 | Bacillus anthracis | 68 | Yes | Yes | Yes | 662 | 993 | 1324 | 1655 |
| WP_098395666.1 | 293 | Bacillus cereus | 8 | No | No | Yes | 663 | 994 | 1325 | 1656 |
| WP_098417350.1 | 294 | Bacillus cereus | 68 | Yes | No | Yes | 664 | 995 | 1326 | 1657 |
| WP_098431974.1 | 295 | Bacillus cereus | 49 | No | No | Yes | 665 | 996 | 1327 | 1658 |
| WP_099032247.1 | 296 | Lactobacillus | 57 | No | No | Yes | 666 | 997 | 1328 | 1659 |
| | | fermentum | | | | | | | | |
| WP_099434208.1 | 297 | Enterococcus faecalis | 79 | Yes | No | Yes | 667 | 998 | 1329 | 1660 |
| WP_099475464.1 | 298 | Listeria monocytogenes | 78 | No | No | Yes | 668 | 999 | 1330 | 1661 |
| WP_099704252.1 | 299 | Enterococcus faecalis | 65 | No | No | Yes | 669 | 1000 | 1331 | 1662 |
| WP_099770130.1 | 300 | Listeria monocytogenes | 78 | No | No | Yes | 670 | 1001 | 1332 | 1663 |
| WP_099890867.1 | 301 | Streptomyces sp. 61 | 11 | Yes | Yes | Yes | 671 | 1002 | 1333 | 1664 |
| WP_100469701.1 | 302 | Mycobacteroides | 55 | Yes | Yes | Yes | 672 | 1003 | 1334 | 1665 |
| | | abscessus subsp. | | | | | | | | |
| | | abscessus | | | | | | | | |
| WP_101933982.1 | 303 | Virgibacillus | 60 | Yes | Yes | Yes | 673 | 1004 | 1335 | 1666 |
| | | dokdonensis | | | | | | | | |
| WP_102135824.1 | 304 | Listeria monocytogenes | 27 | No | No | Yes | 674 | 1005 | 1336 | 1667 |
| WP_102578340.1 | 305 | Listeria monocytogenes | 78 | No | No | Yes | 675 | 1006 | 1337 | 1668 |
| WP_103629687.1 | 306 | Bacillus thuringiensis | 49 | No | No | Yes | 676 | 1007 | 1338 | 1669 |
| | | serovar alesti | | | | | | | | |
| WP_103686139.1 | 307 | Listeria monocytogenes | 78 | No | No | Yes | 677 | 1008 | 1339 | 1670 |
| WP_104869821.1 | 308 | Listeria monocytogenes | 27 | No | No | Yes | 678 | 1009 | 1340 | 1671 |
| WP_105241906.1 | 309 | Shigella dysenteriae | 20 | Yes | No | Yes | 679 | 1010 | 1341 | 1672 |
| WP_107539588.1 | 310 | Staphylococcus | 73 | No | No | Yes | 680 | 1011 | 1342 | 1673 |
| | | simulans | | | | | | | | |
| WP_107639985.1 | 311 | Staphylococcus hominis | 37 | No | No | Yes | 681 | 1012 | 1343 | 1674 |
| WP_109978683.1 | 312 | Streptomyces sp. | 11 | Yes | No | Yes | 682 | 1013 | 1344 | 1675 |
| | | CS090A | | | | | | | | |
| WP_111718485.1 | 313 | Streptococcus | 57 | No | No | Yes | 683 | 1014 | 1345 | 1676 |
| | | pasteurianus | | | | | | | | |
| WP_113850194.1 | 314 | Enterococcus | 79 | Yes | Yes | Yes | 684 | 1015 | 1346 | 1677 |
| | | gallinarum | | | | | | | | |
| WP_113851201.1 | 315 | Enterococcus faecalis | 79 | Yes | No | Yes | 685 | 1016 | 1347 | 1678 |
| WP_113936808.1 | 316 | Bacillus sp. DB-2 | 8 | No | No | Yes | 686 | 1017 | 1348 | 1679 |
| WP_114679402.1 | 317 | Enterococcus faecalis | 65 | No | No | Yes | 687 | 1018 | 1349 | 1680 |
| WP_114980936.1 | 318 | Clostridium botulinum | 21 | No | No | Yes | 688 | 1019 | 1350 | 1681 |
| WP_115205932.1 | 319 | Escherichia coli | 42 | Yes | No | Yes | 689 | 1020 | 1351 | 1682 |
| WP_115261900.1 | 320 | Streptococcus | 54 | No | No | Yes | 690 | 1021 | 1352 | 1683 |
| | | dysgalactiae | | | | | | | | |
| WP_115333169.1 | 321 | Escherichia coli | 1 | Yes | Yes | Yes | 691 | 1022 | 1353 | 1684 |
| WP_115597271.1 | 322 | Corynebacterium | 47 | Yes | Yes | Yes | 692 | 1023 | 1354 | 1685 |
| | | jeikeium | | | | | | | | |
| WP_117232108.1 | 323 | Staphylococcus aureus | 71 | No | No | Yes | 693 | 1024 | 1355 | 1686 |
| | | subsp. aureus | | | | | | | | |
| WP_118991797.1 | 324 | Bacillus thuringiensis | 49 | No | No | Yes | 694 | 1025 | 1356 | 1687 |
| | | LM1212 | | | | | | | | |
| WP_119503980.1 | 325 | Staphylococcus | 73 | No | No | Yes | 695 | 1026 | 1357 | 1688 |
| | | haemolyticus | | | | | | | | |
| WP_120150877.1 | 326 | Listeria monocytogenes | 27 | No | No | Yes | 696 | 1027 | 1358 | 1689 |
| WP_121590887.1 | 327 | Bacillus subtilis subsp. | 36 | No | Yes | Yes | 697 | 1028 | 1359 | 1690 |
| | | subtilis | | | | | | | | |
| WP_123159886.1 | 328 | Streptococcus sp. | 57 | No | No | Yes | 698 | 1029 | 1360 | 1691 |
| | | AM43-2AT | | | | | | | | |
| WP_123257979.1 | 329 | Bacillus circulans | 62 | No | No | Yes | 699 | 1030 | 1361 | 1692 |
| WP_123850201.1 | 330 | Burkholderia | 75 | Yes | No | Yes | 700 | 1031 | 1362 | 1693 |
| | | pseudomallei | | | | | | | | |
| WP_123850205.1 | 331 | Burkholderia | 16 | Yes | No | Yes | 701 | 1032 | 1363 | 1694 |
| | | pseudomallei | | | | | | | | |
| WP_124096936.1 | 332 | Pseudomonas | 5 | No | No | Yes | 702 | 1033 | 1364 | 1695 |
| | | aeruginosa | | | | | | | | |
| WP_124207899.1 | 333 | Pseudomonas | 5 | No | No | Yes | 703 | 1034 | 1365 | 1696 |
| | | aeruginosa | | | | | | | | |
| WP_124982970.1 | 334 | Ralstonia | 5 | No | No | Yes | 704 | 1035 | 1366 | 1697 |
| | | solanacearum | | | | | | | | |
| WP_125180711.1 | 335 | Enterococcus faecalis | 65 | No | No | Yes | 705 | 1036 | 1367 | 1698 |
| WP_125184747.1 | 336 | Streptococcus | 57 | No | No | Yes | 706 | 1037 | 1368 | 1699 |
| | | pneumoniae | | | | | | | | |
| WP_125387060.1 | 337 | Enterobacter asburiae | 4 | Yes | No | Yes | 707 | 1038 | 1369 | 1700 |
| WP_125742262.1 | 338 | Streptomyces sp. | 28 | Yes | Yes | Yes | 708 | 1039 | 1370 | 1701 |
| | | WAC01280 | | | | | | | | |
| WP_128382843.1 | 339 | Staphylococcus | 71 | No | No | Yes | 709 | 1040 | 1371 | 1702 |
| | | schleiferi | | | | | | | | |
| WP_128435673.1 | 340 | Enterococcus hirae | 31 | Yes | No | Yes | 710 | 1041 | 1372 | 1703 |
| WP_128435701.1 | 341 | Enterococcus hirae | 27 | No | No | Yes | 711 | 1042 | 1373 | 1704 |
| WP_129133149.1 | 342 | Clostridium tetani | 23 | Yes | Yes | Yes | 712 | 1043 | 1374 | 1705 |
| WP_129137749.1 | 343 | Bacillus subtilis | 22 | No | Yes | No | | | | |
| WP_129343574.1 | 344 | Enterococcus faecalis | 65 | No | No | Yes | 713 | 1044 | 1375 | 1706 |
| WP_131019985.1 | 345 | Clostridioides difficile | 27 | No | No | Yes | 714 | 1045 | 1376 | 1707 |
| WP_131020076.1 | 346 | Clostridioides difficile | 31 | Yes | No | Yes | 715 | 1046 | 1377 | 1708 |
| WP_131321169.1 | 347 | Burkholderia sp. | 0 | Yes | Yes | Yes | 716 | 1047 | 1378 | 1709 |
| | | WK1.1f | | | | | | | | |
| WP_131931307.1 | 348 | Bacillus thuringiensis | 78 | No | No | Yes | 717 | 1048 | 1379 | 1710 |
| WP_135025396.1 | 349 | Carnobacterium | 54 | No | No | Yes | 718 | 1049 | 1380 | 1711 |
| | | divergens | | | | | | | | |
| WP_136074427.1 | 350 | Streptococcus pyogenes | 85 | No | Yes | Yes | 719 | 1050 | 1381 | 1712 |
| WP_136074428.1 | 351 | Streptococcus pyogenes | 33 | Yes | Yes | Yes | 720 | 1051 | 1382 | 1713 |
| WP_136106493.1 | 352 | Streptococcus pyogenes | 54 | No | No | Yes | 721 | 1052 | 1383 | 1714 |
| WP_136111045.1 | 353 | Streptococcus pyogenes | 54 | No | No | Yes | 722 | 1053 | 1384 | 1715 |
| WP_136118942.1 | 354 | Streptococcus pyogenes | 54 | No | No | Yes | 723 | 1054 | 1385 | 1716 |
| WP_136266174.1 | 355 | Streptococcus pyogenes | 54 | No | No | Yes | 724 | 1055 | 1386 | 1717 |
| YP_001089468.1 | 356 | Clostridioides difficile | 74 | No | No | No | | | | |
| | | 630 | | | | | | | | |
| YP_001271396.1 | 357 | Lactobacillus reuteri | 57 | No | No | No | | | | |
| | | DSM 20016 | | | | | | | | |
| YP_001376196.1 | 358 | Bacillus cytotoxicus | 62 | No | No | No | | | | |
| | | NVH 391-98 | | | | | | | | |
| YP_001384783.1 | 359 | Clostridium botulinum | 8 | No | No | No | | | | |
| | | A str. ATCC 19397 | | | | | | | | |
| YP_001392519.1 | 360 | Clostridium botulinum | 21 | No | Yes | No | | | | |
| | | F str. Langeland | | | | | | | | |
| ΥP_001604091.1 | 361 | Staphylococcus virus | 73 | No | No | No | | | | |
| | | phiMR11 | | | | | | | | |
| ΥP_001646422.1 | 362 | Bacillus | 8 | No | No | No | | | | |
| | | weihenstephanensis | | | | | | | | |
| | | KBAB4 | | | | | | | | |
| ΥP_001886479.1 | 363 | Clostridium botulinum | 81 | No | Yes | No | | | | |
| | | B str. Eklund 17B | | | | | | | | |
| | | (NRP) | | | | | | | | |
| ΥP_002336631.1 | 364 | Bacillus cereus AH187 | 35 | No | No | No | | | | |
| ΥP_002736920.1 | 365 | Streptococcus | 57 | No | No | No | | | | |
| | | pneumoniae JJA | | | | | | | | |
| ΥP_002747001.1 | 366 | Streptococcus equi | 54 | No | No | No | | | | |
| | | subsp. equi 4047 | | | | | | | | |
| ΥP_002804732.1 | 367 | Clostridium botulinum | 24 | No | Yes | No | | | | |
| | | A2 str. Kyoto | | | | | | | | |
| ΥP_003251752.1 | 368 | Geobacillus sp. | 56 | No | No | No | | | | |
| | | Y412MC61 | | | | | | | | |
| ΥP_003358736.1 | 369 | Mycobacterium virus | 32 | No | No | No | | | | |
| | | Peaches | | | | | | | | |
| ΥP_003445547.1 | 370 | Streptococcus mitis B6 | 57 | No | No | No | | | | |
| ΥP_003472505.1 | 371 | Staphylococcus | 73 | No | No | No | | | | |
| | | lugdunensis HKU09-01 | | | | | | | | |
| ΥP_003880342.1 | 372 | Streptococcus | 57 | No | No | No | | | | |
| | | pneumoniae 670-6B | | | | | | | | |
| ΥP_004301563.1 | 373 | Brochothrix phage BL3 | 57 | No | No | No | | | | |
| ΥP_004586821.1 | 374 | Geobacillus | 56 | No | No | No | | | | |
| | | thermoglucosidasius | | | | | | | | |
| | | C56-YS93 | | | | | | | | |
| ΥP_005549228.1 | 375 | Bacillus | 36 | No | No | No | | | | |
| | | amyloliquefaciens XH7 | | | | | | | | |
| ΥP_005679179.1 | 376 | Clostridium botulinum | 8 | No | Yes | No | | | | |
| | | H04402 065 | | | | | | | | |
| ΥP_005759947.1 | 377 | Staphylococcus | 71 | No | No | No | | | | |
| | | lugdunensis N920143 | | | | | | | | |
| ΥP_005869510.1 | 378 | Lactococcus lactis | 54 | No | No | No | | | | |
| | | subsp. lactis CV56 | | | | | | | | |
| ΥP_006082695.1 | 379 | Streptococcus suis D12 | 85 | No | No | No | | | | |
| ΥP_006538656.1 | 380 | Enterococcus faecalis | 65 | No | No | No | | | | |
| | | D32 | | | | | | | | |
| ΥP_006906969.1 | 381 | Streptomyces phage | 17 | No | No | No | | | | |
| | | SV1 | | | | | | | | |
| ΥP_006906969.1 | 382 | Streptomyces | 17 | No | No | Yes | 725 | 1056 | 1387 | 1718 |
| | | venezuelae | | | | | | | | |
| ΥP_006907228.1 | 383 | Streptomyces virus TG1 | 2 | No | Yes | No | | | | |
| ΥP_008050906.1 | 384 | Streptomyces phage | 19 | No | No | No | | | | |
| | | Lika | | | | | | | | |
| ΥP_008051452.1 | 385 | Streptomyces phage | 19 | No | No | No | | | | |
| | | Sujidade | | | | | | | | |
| ΥP_008060284.1 | 386 | Streptomyces phage | 19 | No | No | No | | | | |
| | | Zemlya | | | | | | | | |
| YP_009200991.1 | 387 | Streptomyces phage | 19 | No | No | No | | | | |
| | | Lannister | | | | | | | | |
| YP_009208329.1 | 388 | Streptomyces phage | 66 | No | No | No | | | | |
| | | Amela | | | | | | | | |
| YP_009214300.1 | 389 | Mycobacterium phage | 45 | No | No | No | | | | |
| | | Theia | | | | | | | | |
| YP_009637934.1 | 390 | Mycobacterium virus | 48 | No | Yes | No | | | | |
| | | Benedict | | | | | | | | |
| YP_009638863.1 | 391 | Mycobacterium virus | 45 | No | Yes | No | | | | |
| | | Rebeuca | | | | | | | | |
| YP_189066.1 | 392 | Staphylococcus | 37 | No | Yes | No | | | | |
| | | epidermidis RP62A | | | | | | | | |
| YP_353073.2 | 393 | Rhodobacter | 10 | No | Yes | No | | | | |
| | | sphaeroides 2.4.1 | | | | | | | | |
| YP_706485.1 | 394 | Rhodococcus jostii | 12 | No | Yes | No | | | | |
| | | RHA1 | | | | | | | | |
| YP_950630.1 | 395 | Staphylococcus | 73 | No | No | Yes | 726 | 1057 | 1388 | 1719 |
| | | epidermidis | | | | | | | | |

C = Cluster;
New C = New Cluster;
Cent = Centroid;
New R = New recombinase;
L = attL;
R = attR;
B = attB;
R = attP
⁺Alternative predicted recognition sites are provided in Table 2.
^{T} Thermophilic organism

TABLE 2
Recombinases and cognate recognition sites with alternative recognition sites

| | | Alternative Predicted | | | | Alternative Predicted | | | |
|---|---|---|---|---|---|---|---|---|---|
| | | Recognition Sites | | | | Recognition Sites | | | |
| Protein Accession | | SEQ ID NO: | | | | SEQ ID NO: | | | |
| Number | Organism | L | R | B | P | L | R | B | P |
| WP_005908927.1 | Fusobacterium | 1720 | 1776 | 1832 | 1888 | | | | |
| | nucleatum subsp. | | | | | | | | |
| | animalis F0419 | | | | | | | | |
| WP_069019758.1 | Listeria monocytogenes | 1721 | 1777 | 1833 | 1889 | | | | |
| WP_071661745.1 | Listeria monocytogenes | 1722 | 1778 | 1834 | 1890 | 1944 | 1949 | 1954 | 1959 |
| WP_000286204.1 | Bacillus cereus MSX- | 1723 | 1779 | 1835 | 1891 | | | | |
| | D12 | | | | | | | | |
| WP_000650392.1 | Bacillus thuringiensis | 1724 | 1780 | 1836 | 1892 | | | | |
| | serovar kurstaki str. | | | | | | | | |
| | YBT-1520 | | | | | | | | |
| WP_002475509.1 | Staphylococcus | 1725 | 1781 | 1837 | 1893 | | | | |
| | epidermidis 14.1.R1.SE | | | | | | | | |
| WP_011276651.1 | Staphylococcus | 1726 | 1782 | 1838 | 1894 | | | | |
| | haemolyticus | | | | | | | | |
| | JCSC1435 | | | | | | | | |
| WP_003770016.1 | Listeria innocua | 1727 | 1783 | 1839 | 1895 | | | | |
| WP_131931307.1 | Bacillus thuringiensis | 1728 | 1784 | 1840 | 1896 | | | | |
| WP_059456121.1 | Burkholderia | 1729 | 1785 | 1841 | 1897 | | | | |
| | vietnamiensis | | | | | | | | |
| WP_010990844.1 | Listeria innocua | 1730 | 1786 | 1842 | 1898 | | | | |
| | Clip11262 | | | | | | | | |
| WP_098360688.1 | Bacillus thuringiensis | 1731 | 1787 | 1843 | 1899 | | | | |
| WP_061660420.1 | Bacillus cereus | 1732 | 1788 | 1844 | 1900 | | | | |
| WP_003731150.1 | Listeria monocytogenes | 1733 | 1789 | 1845 | 1901 | | | | |
| WP_097501458.1 | Listeria monocytogenes | 1734 | 1790 | 1846 | 1902 | | | | |
| WP_063280150.1 | Staphylococcus | 1735 | 1791 | 1847 | 1903 | | | | |
| | epidermidis | | | | | | | | |
| WP_053028958.1 | Staphylococcus | 1736 | 1792 | 1848 | 1904 | 1945 | 1950 | 1955 | 1960 |
| | haemolyticus | | | | | | | | |
| WP_002349497.1 | Enterococcus faecium | 1737 | 1793 | 1849 | 1905 | | | | |
| | R501 | | | | | | | | |
| WP_033654380.1 | Enterococcus faecium | 1738 | 1794 | 1850 | 1906 | | | | |
| | R501 | | | | | | | | |
| WP_044791785.1 | Bacillus thuringiensis | 1739 | 1795 | 1851 | 1907 | | | | |
| WP_033943750.1 | Pseudomonas | 1740 | 1796 | 1852 | 1908 | | | | |
| | aeruginosa | | | | | | | | |
| WP_057385580.1 | Pseudomonas | 1741 | 1797 | 1853 | 1909 | | | | |
| | aeruginosa | | | | | | | | |
| WP_011017563.1 | Streptococcus pyogenes | 1742 | 1798 | 1854 | 1910 | | | | |
| | MGAS10270 | | | | | | | | |
| WP_136111045.1 | Streptococcus pyogenes | 1743 | 1799 | 1855 | 1911 | 1946 | 1951 | 1956 | 1961 |
| WP_115261900.1 | Streptococcus | 1744 | 1800 | 1856 | 1912 | | | | |
| | dysgalactiae | | | | | | | | |
| WP_081113934.1 | Bacillus thuringiensis | 1745 | 1801 | 1857 | 1913 | | | | |
| WP_118991797.1 | Bacillus thuringiensis | 1746 | 1802 | 1858 | 1914 | | | | |
| | LM1212 | | | | | | | | |
| WP_015891191.1 | Brevibacillus brevis | 1747 | 1803 | 1859 | 1915 | | | | |
| | NBRC 100599 | | | | | | | | |
| WP_124982970.1 | Ralstonia | 1748 | 1804 | 1860 | 1916 | | | | |
| | solanacearum | | | | | | | | |
| WP_096962681.1 | Escherichia coli | 1749 | 1805 | 1861 | 1917 | | | | |
| WP_021534391.1 | Escherichia coli HVH | 1750 | 1806 | 1862 | 1918 | | | | |
| | 147 (4-5893887) | | | | | | | | |
| WP_037835118.1 | Streptomyces sp. NRRL | 1751 | 1807 | 1863 | 1919 | | | | |
| | S-455 | | | | | | | | |
| WP_002359484.1 | Enterococcus faecalis | 1752 | 1808 | 1864 | 1920 | 1947 | 1952 | 1957 | 1962 |
| WP_002381434.1 | Enterococcus faecalis | 1753 | 1809 | 1865 | 1921 | | | | |
| WP_043503403.1 | Pseudomonas | 1754 | 1810 | 1866 | 1922 | | | | |
| | aeruginosa | | | | | | | | |
| WP_057383473.1 | Pseudomonas | 1755 | 1811 | 1867 | 1923 | | | | |
| | aeruginosa | | | | | | | | |
| WP_002399935.1 | Enterococcus faecalis | 1756 | 1812 | 1868 | 1924 | | | | |
| | TX0309B | | | | | | | | |
| WP_069500683.1 | Bacillus licheniformis | 1757 | 1813 | 1869 | 1925 | | | | |
| WP_079448828.1 | Listeria monocytogenes | 1758 | 1814 | 1870 | 1926 | | | | |
| WP_070030387.1 | Listeria monocytogenes | 1759 | 1815 | 1871 | 1927 | | | | |
| WP_003727736.1 | Listeria monocytogenes | 1760 | 1816 | 1872 | 1928 | | | | |
| | J0161 | | | | | | | | |
| WP_072217376.1 | Listeria monocytogenes | 1761 | 1817 | 1873 | 1929 | | | | |
| WP_113936808.1 | Bacillus sp. DB-2 | 1762 | 1818 | 1874 | 1930 | | | | |
| WP_014636355.1 | Streptococcus suis | 1763 | 1819 | 1875 | 1931 | | | | |
| WP_079253086.1 | Streptococcus suis | 1764 | 1820 | 1876 | 1932 | | | | |
| WP_104869821.1 | Listeria monocytogenes | 1765 | 1821 | 1877 | 1933 | | | | |
| WP_096812886.1 | Listeria monocytogenes | 1766 | 1822 | 1878 | 1934 | | | | |
| WP_014929968.1 | Listeria monocytogenes | 1767 | 1823 | 1879 | 1935 | | | | |
| | FSL N1-017 | | | | | | | | |
| WP_064034122.1 | Listeria monocytogenes | 1768 | 1824 | 1880 | 1936 | | | | |
| WP_102135824.1 | Listeria monocytogenes | 1769 | 1825 | 1881 | 1937 | | | | |
| WP_128435673.1 | Enterococcus hirae | 1770 | 1826 | 1882 | 1938 | | | | |
| WP_128435701.1 | Enterococcus hirae | 1771 | 1827 | 1883 | 1939 | | | | |
| SHX05262.1 | Mycobacteroides | 1772 | 1828 | 1884 | 1940 | | | | |
| | abscessus subsp. | | | | | | | | |
| | abscessus | | | | | | | | |
| WP_131019985.1 | Clostridioides difficile | 1773 | 1829 | 1885 | 1941 | | | | |
| WP_131020076.1 | Clostridioides difficile | 1774 | 1830 | 1886 | 1942 | | | | |
| NP_831691.1 | Bacillus cereus ATCC | 1775 | 1831 | 1887 | 1943 | 1948 | 1953 | 1958 | 1963 |
| | 14579 | | | | | | | | |

### Example 3. Recombinases from Thermophilic Organisms

Presented herein is a group of sequences of recombinases and at least two pairs of DNA target sites (attL/attR; attB/attP) for recombinase genes that were identified from thermophilic organisms. Thermophiles are microorganisms that grow at above-normal temperatures, and thus, proteins identified from thermophilic organisms, are inherently more thermostable than proteins identified from non-thermophilic organisms.

Thermostable enzymes have proven incredibly valuable for biotechnological applications as they allow for enhanced function at elevated temperature. For example, Taq DNA polymerase is a naturally thermostable enzyme that remains functional even after being exposed to near boiling (95° C.+) temperatures and paved the way for the development of PCR. Thermostable recombinase variants are important for generating high-efficiency recombination in both prokaryotic and eukaryotic cells. For example, FlpE—an evolved thermostable variant of the S cerevisae recombinase Flp is more active than the wildtype version, including in bacteria, plants, and mice.

Natural recombinases from thermophilic organisms are therefore important for performing high efficiency recombination over a broad temperature range. Recombinases from thermophiles were identified by the taxonomy of the host organism in which their recognition sites were identified. Newly identified thermophilic recombinase sequences and their DNA targets can be found in Table 1, marked by a “T”.

### Example 4. Site-Specific Recombinases with Innate Nuclear Localization Signal Sequences

Site-specific DNA recombinases evolved to function in prokaryotes, but some of the most impactful applications of DNA recombination are in eukaryotes (e.g., for genome engineering of plants and mammalian cells). For efficient recombination to proceed in eukaryotes, prokaryotic derived recombinases are effectively transported to the nucleus. Certain natural recombinases, such as Cre recombinase, have nuclear localization signals (NLS) inherent in their sequence that allow for their efficient transport into the nucleus. NLS sequences can be also be appended to the N or C terminus of a site-specific recombinase that otherwise does not have a natural NLS-like signal embedded in its sequence. Although engineered recombinase-NLS fusion proteins can then move more efficiently into the nucleus than their wildtype parent, not all recombinases tolerate the NLS fusion and/or exhibit an increased nuclear transport function that puts them on par with natural NLS containing recombinases like Cre.

The publicly available NucPred software (can be accessed at nucpred.bioinfo.se/nucpred/) and the publicly available NLStradamus software (can be accessed at moseslab.csb.utoronto.ca/NLStradamus/) were used to determine if any of the 331 new site-specific recombinases that were identified with described target sites contain NLS-like sequences. NLS-like signal sequences were predicted for proteins that either had a NucPred score >0.8 (Brameier, 2007) or a 2 state HMM static NLStradamus score >0.6 (Nguyen Ba AN, 2009). Herein reported are the identification of 54 site-specific recombinases (from 18 unique clusters) and their associated DNA substrates for recombinases that inherently contain natural NLS-like signals in their amino acid sequences. NLS-containing recombinases and cognate recognition sites are provided in Table 3 (the corresponding recognition sites can be found in Table 1 by matching the Protein Accession Number and Organism).

TABLE 3
NLS-Containing Recombinases

| Protein Accession | |
|---|---|
| Number | Organism |
| WP_003199542.1 | Bacillus pseudomycoides |
| WP_071647453.1 | Clostridium botulinum |
| WP_046655502.1 | Clostridium tetani |
| WP_002349497.1 | Enterococcus faecium R501 |
| EOE27531.1 | Enterococcus faecalis EnGen0285 |
| WP_009269239.1 | Enterococcus faecium |
| WP_079167461.1 | Streptomyces nanshensis |
| WP_129133149.1 | Clostridium tetani |
| WP_038521242.1 | Streptomyces albulus |
| WP_016570474.1 | Streptomyces albulus ZPM |
| WP_003731148.1 | Listeria monocytogenes FSL N1-017 |
| WP_060868949.1 | Listeria monocytogenes |
| WP_128435673.1 | Enterococcus hirae |
| WP_064034122.1 | Listeria monocytogenes |
| WP_077319577.1 | Listeria monocytogenes |
| WP_089602000.1 | Salmonella enterica |
| NP_831691.1 | Bacillus cereus ATCC 14579 |
| WP_000872535.1 | Bacillus cereus BAG3X2-2 |
| WP_000872533.1 | Bacillus sp. 2D03 |
| WP_097877701.1 | Bacillus cereus |
| AND10894.1 | Bacillus thuringiensis serovar alesti |
| WP_081252865.1 | Bacillus thuringiensis serovar alesti |
| WP_098431974.1 | Bacillus cereus |
| WP_103629687.1 | Bacillus thuringiensis serovar alesti |
| WP_081113934.1 | Bacillus thuringiensis |
| WP_001044789.1 | Streptococcus agalactiae CCUG 39096 A |
| WP_065733410.1 | Streptococcus agalactiae |
| WP_083983188.1 | Streptococcus pneumoniae |
| WP_013524454.1 | Geobacillus sp. Y412MC61 |
| WP_123159886.1 | Streptococcus sp. AM43-2AT |
| WP_000633509.1 | Streptococcus pneumoniae 670-6B |
| WP_046559965.1 | Bacillus velezensis |
| WP_052497231.1 | Bacillus thuringiensis serovar morrisoni |
| WP_123257979.1 | Bacillus circulans |
| EOK04340.1 | Enterococcus faecalis EnGen0367 |
| WP_002399935.1 | Enterococcus faecalis TX0309B |
| WP_002409538.1 | Enterococcus faecalis TX0645 |
| WP_002416055.1 | Enterococcus faecalis ERV103 |
| WP_010717149.1 | Enterococcus faecalis EnGen0115 |
| WP_010826647.1 | Enterococcus faecalis EnGen0359 |
| WP_025191276.1 | Enterococcus faecalis EnGen0367 |
| WP_099704252.1 | Enterococcus faecalis |
| WP_002359484.1 | Enterococcus faecalis |
| WP_002381434.1 | Enterococcus faecalis |
| WP_010708035.1 | Enterococcus faecalis EnGen0061 |
| WP_048962262.1 | Enterococcus faecalis |
| WP_077143729.1 | Enterococcus faecalis |
| WP_114679402.1 | Enterococcus faecalis |
| WP_125180711.1 | Enterococcus faecalis |
| WP_129343574.1 | Enterococcus faecalis |
| WP_081225183.1 | Staphylococcus xylosus |
| WP_085707778.1 | Listeria monocytogenes |
| WP_113850194.1 | Enterococcus gallinarum |
| WP_051428004.1 | Paenibacillus larvae subsp. larvae DSM 25719 |

### Example 5. Site-Specific Recombinases with Valuable DNA Target Sequences

Recombinase genes where the DNA target sites themselves were interesting because they do not resemble any known DNA target site for a site-specific recombinase were identified.

Note that site-specific recombinases can be used in an engineered context to recombine at their given target site genomic location in arbitrary engineered nucleic acids (FIG. 4). Because so few site-specific recombinase target sites were previously known (only 64), for most researchers to be able to take advantage of recombinases, they first had (1) laboriously engineer the recombinase target site into a genomic location of choice (2) apply the recombinase to rearrange DNA at the newly added insertion site. Herein are provided site-specific recombinases with recognition sites already present in the genomes of clinically relevant and/or research-based model organisms. These recombinases are valuable because they may be directly applied in the organism that already contains the recombinase recognition sequences without having to perform the initial, laborious target site engineering work (FIG. 5).

Thus, these recombinases, in some embodiments, can be used directly to engineer the genomes of the bacterial organism that contains the identified DNA substrates with no prior engineering work. This is particularly valuable for the introduction of new DNA into a genome (for research, therapeutic or industrial purposes) and especially for organisms that are otherwise challenging to manipulate with current genetic engineering approaches, such as gram-positive bacteria. Co-transformation of an engineered nucleic acid vector that results in the expression of a recombinase and a donor DNA vector that contains one recombinase recognition site could be used to integrate the donor DNA specifically and directly into the natural bacterial genome at the precise location that naturally contains the second recombinase recognition sequence.

Of the 331 characterized site-specific recombinases disclosed here, 62 have DNA target sites in bacteria from genera for which no previously known site-specific recombinase had a target site. These genera are now “unlocked” for direct genome engineering. The 62 site specific recombinases and the genera that they may be used in are provided in Table 4 (the corresponding recognition sites can be found in Table 1 by matching the Protein Accession Number and Organism).

TABLE 4
Recombinase/recognition site pairs of new genera

| Protein Accession | | |
|---|---|---|
| Number | Organism | Genus |
| WP_115597271.1 | Corynebacterium jeikeium | Corynebacterium |
| WP_015407430.1 | Dehalococcoides mccartyi BTF08 | Dehalococcoides |
| WP_015407429.1 | Dehalococcoides mccartyi BTF08 | Dehalococcoides |
| WP_015407431.1 | Dehalococcoides mccartyi BTF08 | Dehalococcoides |
| WP_125387060.1 | Enterobacter asburiae | Enterobacter |
| KDF51021.1 | Enterobacter roggenkampii CHS 79 | Enterobacter |
| WP_115333169.1 | Escherichia coli | Escherichia |
| WP_024233971.1 | Escherichia coli STEC O174:H46 str. 1-151 | Escherichia |
| WP_053903616.1 | Escherichia coli | Escherichia |
| GDD80774.1 | Escherichia coli | Escherichia |
| WP_061355600.1 | Escherichia coli | Escherichia |
| WP_096962681.1 | Escherichia coli | Escherichia |
| WP_021534391.1 | Escherichia coli HVH 147 (4-5893887) | Escherichia |
| WP_115205932.1 | Escherichia coli | Escherichia |
| WP_000709069.1 | Escherichia coli 5.0588 | Escherichia |
| WP_000709099.1 | Escherichia coli 55989 | Escherichia |
| WP_070080197.1 | Escherichia coli O157:H7 | Escherichia |
| NP_415076.1 | Escherichia coli str. K-12 substr. MG1655 | Escherichia |
| WP_008698549.1 | Fusobacterium ulcerans 12-1B | Fusobacterium |
| WP_060798679.1 | Fusobacterium nucleatum | Fusobacterium |
| WP_005908927.1 | Fusobacterium nucleatum subsp. animalis F0419 | Fusobacterium |
| WP_008700773.1 | Fusobacterium nucleatum subsp. polymorphum F0401 | Fusobacterium |
| EFD80439.2 | Fusobacterium nucleatum subsp. animalis D11 | Fusobacterium |
| WP_045667426.1 | Geobacter sulfurreducens | Geobacter |
| WP_003514343.1 | Hungateiclostridium thermocellum JW20 | Hungateiclostridium |
| WP_089997567.1 | Leuconostoc gelidum subsp. gasicomitatum | Leuconostoc |
| WP_069482207.1 | Lysinibacillus fusiformis | Lysinibacillus |
| WP_100469701.1 | Mycobacteroides abscessus subsp. abscessus | Mycobacteroides |
| SHX05262.1 | Mycobacteroides abscessus subsp. abscessus | Mycobacteroides |
| WP_082870750.1 | Nocardia terpenica | Nocardia |
| WP_115597271.1 | Corvnebacterium jeikeium | Corvnebacterium |
| WP_071218019.1 | Paenibacillus sp. LC231 | Paenibacillus |
| WP_064963684.1 | Paenibacillus polymvxa | Paenibacillus |
| WP_051428004.1 | Paenibacillus larvae subsp. larvae DSM 25719 | Paenibacillus |
| WP_039660878.1 | Pantoea sp. MBLJ3 | Pantoea |
| WP_031673611.1 | Pseudomonas aeruginosa | Pseudomonas |
| WP_033943750.1 | Pseudomonas aeruginosa | Pseudomonas |
| WP_043503403.1 | Pseudomonas aeruginosa | Pseudomonas |
| WP_057383473.1 | Pseudomonas aeruginosa | Pseudomonas |
| WP_057385580.1 | Pseudomonas aeruginosa | Pseudomonas |
| WP_058016331.1 | Pseudomonas aeruginosa | Pseudomonas |
| WP_074196983.1 | Pseudomonas aeruginosa | Pseudomonas |
| WP_124096936.1 | Pseudomonas aeruginosa | Pseudomonas |
| WP_124207899.1 | Pseudomonas aeruginosa | Pseudomonas |
| WP_019725860.1 | Pseudomonas aeruginosa 213BR | Pseudomonas |
| WP_023107160.1 | Pseudomonas aeruginosa BL04 | Pseudomonas |
| WP_023115516.1 | Pseudomonas aeruginosa BWHPSA021 | Pseudomonas |
| WP_073656076.1 | Pseudomonas aeruginosa | Pseudomonas |
| WP_073656028.1 | Pseudomonas aeruginosa | Pseudomonas |
| WP_064297673.1 | Ralstonia solanacearum | Ralstonia |
| WP_124982970.1 | Ralstonia solanacearum | Ralstonia |
| WP_089602000.1 | Salmonella enterica | Salmonella |
| WP_001233549.1 | Shigella boydii | Shigella |
| WP_105241906.1 | Shigella dysenteriae | Shigella |
| WP_094146498.1 | Shigella sonnei | Shigella |
| WP_066864475.1 | Sphingobium sp. TCM1 | Sphingobium |
| WP_085430121.1 | Sporosarcina sp. P37 | Sporosarcina |
| WP_053497239.1 | Stenotrophomonas maltophilia | Stenotrophomonas |
| WP_065724346.1 | Stenotrophomonas maltophilia | Stenotrophomonas |
| KIS38487.1 | Stenotrophomonas maltophilia WJ66 | Stenotrophomonas |
| WP_028992649.1 | Thermoanaerobacter thermocopriae JCM 7501 | Thermoanaerobacter |
| WP_101933982.1 | Virgibacillus dokdonensis | Virgibacillus |
| WP_044751504.1 | Xanthomonas oryzae pv. oryzicola | Xanthomonas |

### Sequence Listing

TABLE 5

| SEQ | |
|---|---|
| ID | |
| NO: | Amino acid Sequence |
| 1 | MKRAALYIRVSTMEQAKEGYSIPAQTDKLKAFAKAKDMAVAKVYTDPGFSGAKMERPALQEMIS |
| | DIQNKKIDVVLVYKLDRLSRSQKNTLYLIEDVFLKNNVDFISMQESFDTSTPFGRATIGMLSVFAQL |
| | ERDTITERMHMGRTERAKQGYYHGSGIVPLGYDYVHGELIINDYEAQIIQEIYDLYVNQGKGQQYI |
| | TKRMVAKYPDKVKTLTIVKYALTNPLYIGKISWDGKVYDGHHSPIIDKSMYDKAQEIIARMAQKG |
| | GEQHGNQLGLLLGITYCGKCGAEVFRYVSGGKKYRYNYYMCRSVKKMLPSLVKDWNCKQPSLR |
| | QEVVEKKVIDSLKSLDFKKIERELKQVENKTKSKITTINNQISKKHNEKQKILDLYQYGTFDVTMLN |
| | ERMKKIDNEINALTANIANLEGTKSESLINKLETLKTFNWETETTENKILIIKEFVERIELFDDEVIIKY |
| | KF |
| 2 | MRALVVIRLSRVTDATTSPERQLESCQQLCAQRGWDVVGVAEDLDVSGAVDPFDRKRRPNLARW |
| | LAFEEQPFDVIVAYRVDRLTRSIRHLQQLVHWAEDHKKLVVSATEAHFDTTTPFAAVVIALMGTVA |
| | QMELEAIKERNRSAAHFNIRAGKYRGSLPPWGYLPTRVDGEWRLVPDPVQRERILEVYHRVVDNH |
| | EPLHLVAHDLNRRGVLSPKDYFAQLQGREPQGREWSATALKRSMISEAMLGYATLNGKTVRDDD |
| | GAPLVRAEPILTREQLEALRAELVKTSRAKPAVSTPSLLLRVLFCAVCGEPAYKFAGGGRKHPRYR |
| | CRSMGFPKHCGNGTVAMAEWDAFCEEQVLDLLGDAERLEKVWVAGSDSAVELAEVNAELVDLT |
| | SLIGSPAYRAGSPQREALDARIAALAARQEELEGLEARPSGWEWRETGQRFGDWWREQDTAAKNT |
| | WLRSMNVRLTFDVRGGLTRTIDFGDLQEYEQHLRLGSVVERLHTGMS |
| 3 | MKYAVYVRVSTDRDEQVSSVENQIDICRYWLEKNGYEWDPNAVYFDDGISGTAWLERHAMQLIL |
| | EKARRNELDTVVFKSIHRLARDLRDALEIKEILIGHGIRLVTIEENYDSLYEGGNDIKFEMFAMFAAQ |
| | LPKTISVSVSAAMQAKARRGEFIGKPGLGYDVIDKKLVINEKEAEIVREIFDLSYKGYGFKKIANILN |
| | DKGTYTKFGQLWSHTTVGKILKNQTYKGNLVLNSYKTVKVDGKKKRVYTPKERLTIIEDHYPTIVS |
| | KELWNAVNSDRASKKKTKQDTRNEFRGMMFCKHCGEPITAKYSGRYAKGSKKEWVYMKCSNYI |
| | RFNRCVNFDPAHYDDIREAIIYGLKQQEKELEIHFNPKMHQKRNDKSTEIKKQIKLLKVKKEKLIDL |
| | YVEGLIDKEMFSKRDLNFENEIKEQELALLKLTDQNKRNKEEKKIKEAFSMLDEEKDMHEVFKTLI |
| | KKITLSKDKYIDIEYTFSL |
| 4 | MNLMDENTPKNVGIYVRVSTEEQAKEGYSISAQKEKLKAYCISQGWDSYKFYIDEGKSAKDIHRPS |
| | LELMLRHIEQGIIDTVLVYRLDRLTRSVRDLYSLLDYFDKYQAVFRSATEVYDTGSATGRLFITLVA |
| | AMAQWERENLGERVKMGQVEKARQGQFSAPAPFGFTKEGESLVKNPEEGEVLLDMIDKIKKGYS |
| | LRELADYLDESDAIPKRGYKWHIASILVILKNPVLYGGFRWAGEILEGAFEGYISKKEFEQLQKMLH |
| | DRQNFKRRETSSIFIFQAKILCPNCGSRLTCERSIYFRKKDNKNVESNHYRCQACALNKKPAIGISEK |
| | KFEKALIEYMQNANFKREPKIPQEKQQDYDKLHQKIISIEKQRKKYQKAWSMELMTDQEFEQLMA |
| | ETKEALQKALAKLEQNDLHPIEKPLNIERAKELAKMFRENWSVLTGEEKRQTVQELIKHIEFEKKD |
| | NKARILDIHFY |
| 5 | MTISGGTDEALFYFRISLDATGERLGVERQEPPCLELCRSKGFTPGKAYIDNDLSATKEGVVRPEFE |
| | ALLRDLKLRPRPVIVWHTDRLVRVTKDLERVISTGVNVYAVHAGHFDLSTPAGRAVARTLTAWAQ |
| | YEGEQKALRQKEANLQRAQMGKPWWPRRPFGLEKDGELNEPEALSLRKAYADLLSGASLTDLAA |
| | DLNAAGHTTNKGGAWTSTSLRPVLMNARNAAIRTYDGEEIGPANWKAIVPEETWRAAVRLLSSPS |
| | RKTGGGGKRLHLMTGVAKCSVCDSDVKVEWRGKKGEPTAYTVYACRGKHCLSHRQKWVDDRV |
| | ETLVLERLSQEDAAAVWAVDNDTELADVREEVVTMRERLEAFAEDYADGAISRAQMQAGSARVR |
| | EKLEAAEAQMAYLAAGSPLGELIASNDVEKTWESLTLDRKRAVIEAMTRKVTLYPRGRGIRSHRPE |
| | DCQVEWVDERPRLSAVS |
| 6 | MAYAVYVRVSSDKDEQVSSVENQIDICRYWLENNGFEWDENAVYFDDGISGTAWLERHAIQLVLE |
| | KARKKEIDTVVFKSIHRLARDLKDALEIKEILLGHGVRLITIEEGYDSHYEGKNDMKFEMYAMFAS |
| | QLPKTLSVSITAALAAKVRRGGYTGGFVPYGYEIIDGKYAINEEEAALVREIFELYAQGFGYIKIANT |
| | INDKGARTRKGAPWTFSTLSKMIKNPAYKGTYIMQKYGTVKVNGRKKKVINPKEKWVIFEGHHPA |
| | IISHELWEKVNNKDPNKFKKKRRVSTTNELRGITVCAHCGTAMSKRNSINVSKNGRETEYSYMICN |
| | WSRITARRECVRHVPIHYKDLRALVLSKLKEKERELDKEFCSDENQLQVKLRKLKKDINDLKFKRE |
| | RLLDLYLEDERIDKDTFTIRNAKIEKEIGLKEMEIRKASNIEIQMKEKQEVRDAFALLEESKDLHSVF |
| | QKLIKRIEVAQDGAIDIYYRFEE |
| 7 | MWACSHLRADGTTPTSSSTLLTMSARDYDIEAEWTPADLALLKELEEAEALLPADAPRALLSVRLS |
| | VFTDDTTSPVRQELDLRQLAREKGHRVVGLASDLNVSATKVPPWKRKSLGDWLNNRAPEFDALLF |
| | WKIDRFIRNLNDLNVMIRWSETYSKNLISKNDPIDLTTTMGKMMVSLLGGVAEIEAANTKTRVESL |
| | WDYTKTQGEWHVGKPPFGYKTARDEAGKVVLVEDPLAVETLHTARELVMSGMSTTAAAKVLKE |
| | RGLISSTTATLTRRLRNPGVLGLRVEEDKDGGIRRSKLILGRDGQPIRIADPIFTEEQFEELQAVLDKR |
| | GKRQPHRQPGGATSFLGVLKCAVCETNMINHYTRNRHGDYAYLRCQGCKSGGYGAPNPQEVYDR |
| | LVEQVLAVLGDFPVEMREYARGEEKRKELKRLEESIAYYMKELEPGGRFTKTRFTQDQAEGTLDK |
| | LIAELEAIDPESAKDRWVYVAGGKTFREHWEEGGIDAMSADLIRAGIMCQVTRTKVPKVRAPQVH |
| | LKLMIPKDVRTRLVIRPDDFGQTF |
| 8 | MSKRAVIYTRVSRDDTGEGQSNQRQEAECRRLTDYRRLDVVAVEADISISASKGLERPAWLRVLG |
| | MIERGEVDYVIAYHMDRVTRSMTELEQLIEMCLKYDVGVATVSGDIDLTTDVGRMVARIIGAVAR |
| | AEVERKSARQKLANAQRAAEGKPHVSGIRPFGYADDHRQVVTIEAQAIRAAAEAALAGESMIGIAE |
| | SWSKDGLLSARARRGHDKGNRPTKAAWSARGVRNVLVNPRYAGIRFYNGERVGQGDWEPILDVE |
| | THLRLVEKLTDPTRRKGTVKTGRVAASLLTAIARCEVCGQTVRASSVRGRQTYACRNSHAHVDRS |
| | TADLMTQEWVISRLADPDTLAKLAPSGDDRVDEAKATIEKRREALKTYARLLATGAMDEDQFTEA |
| | SAVARSEMQEAEAVLTEAGTGDLLAGLDVGSDAVGPQFLALSLARQRGIVEALVDVTLRPASKAR |
| | KVVTPEHERVILADR |
| 9 | MKYAVYVRVSTDRDEQVSSIENQIDICRYWIEKNGYEWDENSIYKDEAVSGTAWLERRAMQLILG |
| | KARKKELDTVVFKSIHRLGRDLRDALEIKEILLGHGVRLVTIEEGYDSYYEGKNDLKFEMYAMFAS |
| | QLPKTLSVSISAALAAKVRRGEYTGGTVPYGYKIVDKKYVINQEEAEIVREMYELYDNGLGYLRIS |
| | NALNDVGKYKRSGKLWTYSAVKLIITNPMYKGDYVMGRSTEVKVDGRKKRIQEPREKWVVFENH |
| | HPAIIERPLWDKINNPKINKKIKRRVAVTNELRGIARCIHCGSPFVLHTYKYKNKEGEELNYGYLTC |
| | GTYKLTGGRGCVKHSGLRYERLRSLVLRKLKEKERDLEKVFKLNDKDKHQEKQKKLRKEKKELEI |
| | KRERLLDLYLDGGSIDKETFTKRDANFAKNIKEKELEILKLDDVKALIVEQQKVKDAFKLLEDSENL |
| | YPVFKKLIAGIDISQNGAVDIRYRFEE |
| 10 | MSNRLHEYDVEAEWSPADLALLRSLEEAESLLPESAPRALLSVRLSVFTEDTTSPVRQELDLRQLAR |
| | DKGMRVVGVASDLNVSATKVPPWKRKSLGTWLNDRVPEFDALLFWKVDRFIRNMSDLSRMIDWS |
| | NRYEKNLISKNDPIDLSTPLGKMMVTLLGGIAEIEAANTKARVESLWDYNKTQSEWLVGKPPYGYT |
| | TARDEQGKNRLVIDPKASEALHLTRLHLLEGGSVRSFVPVLKEKGLVSTGLTPSTLIRRLRNPALLG |
| | YRVEEDKKGGLRRSKVVVGHDGQPIVIADPIFTREEWDTLQAAMDARNKNQPPRQPSGATKFRGV |
| | LKCVECGTNMIVHHTRNKHGEYAYLRCQGCQSGGLGSPHPQDVYDALVGQVLTVLGDWPVQTRE |
| | YARGAEARAETKRLEETIAVYMKGLEPGGRYTKTRFTMEQAEATLDKLIAELEAIDPDTTTDRWV |
| | YVAGGKTFREHWEEGGMDAMTSDLLRAGITATVTRTKIPKVRAPKVELDLDIPKDVRERLIVREDD |
| | FAETF |
| 11 | MNYERSYLRSCQVSTLEQKEHGYSIEEQERKLKSFCEINDWSISDVFIDAGFSGAKRDRPELQRMM |
| | KDIKRFDLVLVYKLDRLTRNVRDLLDLLEIFEQNNVAFRSATEVYDTSTAMGRLFVTLVGAMAEW |
| | ERETIRERVMMGKRAAIKQGMILTPPPFYYDRVDNTYIPNDYKKVVLWAYDEVLKGVSSKGIARK |
| | LNDSDIPPPNGKRWEDRTITRALRSPITRGHYTWGDVFIENSHEPIITEEMYQQIKERLEERINTKIVS |
| | HVSVFRGKFICPRCGGTLTMNTATRKRKKGYVTYKTYYCNTCKGKKESFGFAENEALRVFRDYLS |
| | KLDLDKYEVKTKQKDDVVTIDIDKVMEQRKRYHKLYAKGLMQEEELFELIKETDETIAEYEKQKE |
| | LVPRKTLDVDKIKKFKNVLLESWKIFSSEDKADFIKMAIKSIDIEYVKFKNRHSIKINDIEFY |
| 12 | MNRGGPTVRADIYVRISLDRTGEELGVERQEESCRELCKSLGMEVGQVWVDNDLSATKKNVVRP |
| | DFEAMIASNPQAIVCWHTDRLIRVTRDLERVIDLGVNVHAVMAGHLDLSTPAGRAVARTVTAWAT |
| | YEGEQKAERQKLANIQNARAGKPYTPGIRPFGYGDDHMTIVTAEADAIRDGAKMILDGWSLSAVA |
| | RYWEELKLQSPRSMAAGGKGWSLRGVKKVLTSPRYVGRSSYLGEVVGDAQWPPILDPDVYYGVV |
| | AILNNPDRFSGGPRTGRTPGTLLAGIALCGECGKTVSGRGYRGVLVYGCKDTHTRTPRSIADGRASS |
| | STLARLMFPDFLPGLLASGQAEDGQSAASKHSEAQTLRERLDGLATAYAEGAISLSQMTAGSEALR |
| | KKLEVIEADLVGSAGIPPFDPVAGVAGLISGWPTTPLPTRRAWVDFCLVVTLNTQKGRHASSMTVD |
| | DHVTIEWRDVAE |
| 13 | MKVAVYCRVSTLEQKEHGHSIEEQERKLKSFCDINDWTVYDTYIDAGYSGAKRDRPELQRLMNDI |
| | NKFDLVLVYKLDRLTRNVRDLLDLLEIFEKNDVSFRSATEVYDTTTAMGRLFVTLVGAMAEWERE |
| | TIRERTQMGKLAALRKGIMLTTPPFYYDRVDNKFVPNKYKDVILWAYDEAMKGQSAKAIARKLN |
| | NSDIPPPNNTQWQGRTITHALRNPFTRGHFDWGGVHIENNHEPIITDEMYEKVKDRLNERVNTKKV |
| | RHTSIFRGKLVCPVCNARLTLNSHKKKSNSGYIFVKQYYCNNCKVTPNLKPVYIKEKEVIKVFYNY |
| | LKRFDLEKYEVTQKQNEPEITIDINKVMEQRKRYHKLYASGLMQEDELFDLIKETDQTIAEYEKQN |
| | ENREVKQYDIEDIKQYKDLLLEMWDISSDEDKEDFIKMAIKNIYFEYIIGTGNTSRKRNSLKITSIEFY |
| 14 | MPGMTTETGPDPAGLIDLFCRKSKAVKSRANGAGQRRKQEISIAAQETLGRKVAALLGMQVRHV |
| | WKEVGSASRFRKGKARDDQSKALKALESGEVGALWCYRLDRWDRGGAGAILKIIEPEDGMPRRL |
| | LFGWDEDTGRPVLDSTNKRDRGELIRRAEEAREEAEKLSERVRDTKAHQRENGEWVNARAPYGLR |
| | VVLVTVSDEEGDEYDERKLAADDEDAGGPDGLTKAEAARLVFTLPVTDRLSYAGTAHAMNTREIP |
| | SPTGGPWIAVTVRDMIQNPAYAGWQTTGRQDGKQRRLTFYNGEGKRVSVMHGPPLVTDEEQEAA |
| | KAAVKGEDGVGVPLDGSDHDTRRKHLLSGRMRCPGCGGSCSYSGNGYRCWRSSVKGGCPAPTYV |
| | ARKSVEEYVAFRWAAKLAASEPDDPFVIAVADRWAALTHPQASEDEKYAKAAVREAEKNLGRLL |
| | RDRQNGVYDGPAEQFFAPAYQEALSTLQAAKDAVSESSASAAVDVSWIVDSSDYEELWLRATPTM |
| | RNAIIDTCIDEIWVAKGQRGRPFDGDERVKIKWAART |
| 15 | MKVAIYTRVSTLEQKEKGHSIEEQERKLRAYSDINDWKIHKVYTDAGYSGAKKDRPALQEMLNEI |
| | DNFDLVLVYKLDRLTRSVKDLLEILELFENKNVLFRSATEVYDTTSAMGRLFVTLVGAMAEWERT |
| | TIQERTAMGRRASARKGLAKTVPPFYYDRVNDKFVPNEYKKVLRFAVEEAKKGTSLREITIKLNNS |
| | KYKAPLGKNWHRSVIGNALTSPVARGHLVFGDIFVENTHEAIISEEEYEEIKLRISEKTNSTIVKHNAI |
| | FRSKLLCPNCNQKLTLNTVKHTPKNKEVWYSKLYFCSNCKNTKNKNACNIDEGEVLKQFYNYLK |
| | QFDLTSYKIENQPKEIEDVGIDIEKLRKERARCQTLFIEGMMDKDEAFPIISRIDKEIHEYEKRKDNDK |
| | GKTFNYEKIKNFKYSLLNGWELMEDELKTEFIKMAIKNIHFEYVKGIKGKRQNSLKITGIEFY |
| 16 | MQLDATLTLRDEGLSAFHQRHIKQGALGVFLRAIEDGRIQPGSVLIVEGLDRLSRAEPIQAQAQLAQ |
| | IINAGITVVTASDGREYNRERLKAQPMDLVYSLLVMIRAHEESDTKSKRVKAAIRRQCEGWVAGT |
| | WRGIIRNGKDPHWVRLGEHGKFEHVPERVLAVRTMIDLFLEGHGAIEITRRLTEQNLYVSNAGNYS |
| | VHMYRIVRNQALIGEKRISVDGEEFRLDGYYPPILTREEFAELQQTMSERGRRKGKGEIPNIITGLSIT |
| | VCGYCGRAMTTQNSKARAPKGKSVVRRLSCPMNSFNEGCPIGGSCESEIVERALMRYCSDQFNLSR |
| | LLEGDDGTARRTAQLAVARQRASDIEAQIQRVTDALLSDDGKAPAAFTRRARELETQLEEQRREIE |
| | ALEHQIAASSAHGIPAAAEAWAQLVDGVLALDYDARMKARQLVADTFRKIVVYQRGFAPIDDAA |
| | ADRWKRSGTIGLMLVTKRGGMRLLNVDRRTGCWQAEDDLDPSLIPSDGLPMLPLDA |
| 17 | MKAAIYIRVSTQEQIENYSIQAQTEKLTALCRSKDWDVYDIFIDGGYSGSNMNRPALNEMLSKLHEI |
| | DAVVVYRLDRLSRSQRDTITLIEEYFLKNNVEFVSLSETLDTSSPFGRAMIGILSVFAQLERETIRDR |
| | MVMGKIKRIEAGLPLTTAKGRTFGYDVIDTKLYINEEEAKQLQMIYDIFEEEKSITSLQKRLKKLGF |
| | KVKSYSSYNNWLTNDLYCGYVSYADKVHTKGVHEPIISEEQFYRVQEIFSRMGKNPNMNRDSASL |
| | LNNLVVCSKCGLGFVHRRKDTVSRGKKYHYRYYSCKTYKHTHELEKCGNKIWRADKLEELIIDRV |
| | NNYSFASRNVDKEDELDNLNEKLKTEHTKKKRLFDLYISGSYEVSELDAMMADIDAQINYYEAQIE |
| | ANEELKKNKKIQENLADLATVDFDSLEFREKQLYLKSLINKIYIDGEQVTIEWL |
| 18 | MGKSITVIPAKKVQTSVLHQDRKKIKVAAYCRVSTDQEEQLSSYENQVNYYREFISKHEDYELVDI |
| | YADEGISATNTKKRDAFNRLIQDCRAGKVDRILVKSISRFARNTLDCIKYVRELKELGVGVTFEKEN |
| | IDSLDSKGEVLLTILSSLAQDESRSISENATWGIRKKFERGEVRVNTTKFMGYDKDENGRLIINPQQA |
| | ETVKFIYEKFLEGYSPESIAKYLNDNEIPGWTGKANWYPSAIQKMLQNEKYKGDALLQKTFTVDFL |
| | TKKRVQNDGQVNQYYVENSHEAIIDEETWETVQLEMARRKTYRDEHQLKSYIMQSEDNPFTTKVF |
| | CGACGSAFGRKNWATSRGKRKVWQCNNRYRIKGVEGCYSSHLDEATLEQIFLKALELLSENIDLL |
| | DGKWEKILAENRLLDKHYSMALSDLLRQEQIDFNPSDMCRVLDHIRIGLDGEITVCLLEGTEVDL |
| 19 | MPIAPEFLSLAYPGQEFPAYLYGRASRDPKRKGRSVQSQLDEGRATCLDAGWPIAGEFKDVDRSAS |
| | AYARRTRDEFEEMIAGIQAGECRILVAFEASRYYRDLEAYVRLRRVCREAGVLLCYNGQVYDLSK |
| | SADRKATAQDAVNAEGEADDIRERNLRTTRLNAKRGGAHGPVPDGYKRRYDPDSGDLVDQIPHP |
| | DRAGLITEIFRRAAAAEPLAAICRDLNERGETTHRGKAWQRHHLHAILRNPAYIGHRRHLGVDTGK |
| | GMWAPICDDEDFAETFQAVQEILSLPGRQLSPGPEAQHLQTGIALCGEHPDEPPLRSVTVRGRTNYN |
| | CSTRYDVAMREDRMDAFVEESVITWLASDEAVAAFEDNTDDERTRKARIRLKVLEEQLEAAQKQA |
| | RTLRPDGMGMLLSIDSLAGLEAELTPQIDKARQESRSLHVPALLRDLLGKPRADVDRAWNEALTLP |
| | QRRMILRMVVTIRLFKAGSRGVRAIEPGRITLSYVGEPGFKPVGGNRAKQ |
| 20 | MDRNKVAIYVRVSTQGQVDDGYSLDEQVDLLTNYCKLKEWTLYDVYVDPGISGKNMHRPEIERL |
| | TRDAKRKLFDIVLIYDLKRLGRSQKENIVLVEDVFNPNGIRLVSFTENFDASTPVGKMVFGMLSAY |
| | AELDRANIAERMMMGKIGRAKAGKAMSWGMPPFAYDYNKETGDLELDEVKAPIVEMIYSEFLKG |
| | ASVNKIVQKLNSMSYHGKNHEWKHHAVTVIIDNPVYCGMMKYMGQTYQAKHTPIIDKKTFELAQ |
| | LERKKRLSKYHDADWLGPFQRKYIGSKICYCGLCGAHLKSEKDKKNKLTGIRSISFFCPNTRSRGTG |
| | ECTNPRFKQSVLEGYILNEVAKLQQNPEKLKDIKPAEDNELHNKIATYEKKIKQNSSKLSKLNDLYL |
| | NDLISLDDLKQQSKSLLNENEFMEEQIKLLSATTREDELRKKIDTFLAFPDILTADYDTQKQAVELVI |
| | SRVEATKEGIDIFFNF |
| 21 | MINVVGYARYSSDNQREESIVAQERAIREFCQKNNYNLIKVYKDEAISGTSIKDRTEFLELIEDSKKK |
| | EFQCVVVHKFDRFARNRYDHAIYEKKLNDNGVKLLSVLEQLNDSPESVILKSVLTGMNEYYSLNLS |
| | REVKKGLNENALNCIHNGGIPPLGYNLDEDRRYIINEIEAETVRIIYKLYIEGIGYASIAEQLNQMGRL |
| | NKLGKPFRKTSIRDILLNEKYTGVFVYGKKDGHGKLTGNEVKIEGGIPQIISKEDFEKIQIKMKNRKT |
| | GSRATAHETYYLTGVCTCGECGGRYSGGYRSRQRDGSITYGYTCINRKTKVNDCRNKPIRKEILEE |
| | FVFKTIKKKYLQKRG |
| 22 | MKKITKIDELPQGQLPNTNLRVAAYARVSTDSDEQLESLKAQREHYERYIKSNPEWEFAGLYYDEG |
| | ISGTKMEKRTELLRMIRNCKQGRIDFIITKSISRFARNTVDCLELVRKLIDIGVYIYFEKENLNTGDME |
| | SELMLSILSGFAAEESASISQNSTWSIQKRFQNGSYVGTPPYGYTNTDGEMVIVPEEAEIIKRIFTECL |
| | SGKGGGTIARGLNKDKIPARRGNHWSAGTVIDMLRNEKYMGDVLLQKTYTDSNYNRHPNTGEKD |
| | QYYYKDSHEAIISREDFAKAQDLIDERAKMKCKGVKKNVYLNRYALSGKIVCGECGRNFRRKTNY |
| | SAGRSYIAWSCIGHIEDKESCSMLFLRDGEIKATFTTMMNKLAFSNKLILEPLFKSISQIDEESDRER |
| | MDAIDKRMEQLMEERNTLITLMAKGFLEPALFNQERNVLDSEIKNLTTEKTNLVTNSTSGVLRAND |
| | IKDLIDYVSADNFNGDYTEELFEEFVENIIVNSRDELTFNLKCGLSLKEKVVR |
| 23 | MKVIQKIEPTKPKIAKRKRVAAYARVSVDKGRTMHSLSAQVSYYSKLIQKNPDWEYVGVYSDGGI |
| | SGRTTESRNEFKRLIKDCKDGKVDIILTKSISRFARNTVDLLETVRDLRAINVEVRFEKENIHSLSGD |
| | GELMLSILASFAQEESRSISNNIKWSIQKRFKEGKHNGRFNIYGYRWVGQELIVEPSEAENIKLMYA |
| | NYMNGLSAEFTAKQLTKMGVTAMKGGPFKATSVRQILKNITYTGNLLLQKEYTPDPITGKSRYNN |
| | GEMPQYFVENHHEAIIPMEEWQAVQDERLKRRKLGAHANKSINTTCFTSKIKCGNCGKNFRRSGK |
| | RQGKNKELYHIWTCRNKSEKGVKVCNARNIPEPALKKYATEVLGLEVFDEQIFIDSIEEIVASEGNM |
| | LQFKFYGGREVEVKWTSTARKDYWTPEVRRAWSERNKRKESRTWNGRTTEFTGFVVCGRCGAN |
| | YRRQAVTSKTDGTVRRKWHCSNSAVACNEGKSRNCIYEEDLKVMVAEILGIPTFNEPTMDEKLSRI |
| | SIIDTEVTFHFKDGHDEVRTFEIPKKKARTFSEEERARRRLVMKKRWEEKKRDEESNNDTSDNH |
| 24 | MDKKAAIYIRVSTQEQATEGYSIQAQTDRLTKYVEAKDFILYKKYIDAGYSASKLERPAMQELIQD |
| | VQSKKVDVVIVYKLDRLSRSQKDTMYLIEDIFRPNDVELISMQESFDTSTAFGSATVGMLSVFAQLE |
| | RKSISERMITGRVERAKKGFYHTGGQDRPPAGYQFNSDNQLIINEYEAAAIKDLFRLYNDGLGKSSI |
| | SEYLKKNYPGKNKWLPSSIDRMLKNSLYIGKVKFSGAEYDGIHEPIIDEVTFYKTQKEIARRKQTNT |
| | KRYNYVALLGGLCECGICGAKMANRRAVGRKGKVYRYYRCYSKKGSPKHMMKTDGCSSKAQQ |
| | QFIIDEAVINNLKNIDVEAELKRRSAPQTNTSLISSQIESIDKQINKLIDLFQVDSMPLDVISEKIDKLN |
| | KEKQSMEKLLERKNKLDKTELQHRFDVLKSFDWDNSSIESKRVVIEMLVQKVIIHDNSIEIILVE |
| 25 | MTTGIYIRVSTEEQAKEGYSIANQKEKLIAFCESQGWSSYKIYSDEGYSAKDMKRPALQEMFNDMT |
| | QGVIKIILVYKLDRLTRSVRDLYTMLETFDKHDCKFKSATEVYDTTTAMGRLFITLVAALAQWERE |
| | NTAERVRVVMENNVKNGKWKGGTLAYGYQLKNGNIVINEDEAATVSFIFNKIKFTGPLAIVRELIK |
| | KNIPTRTGSDWHVDTIRGIITNPFYIGYQRFNDSLKQYKGSVKQQKLYKSSHESIISEDEFWEVQEIL |
| | NARKTHGSKKSTSTYYFSTVLTCGVCGASMCGHLSGNKKTYRCNKKKTSGNCDSSLILESTIVNWL |
| | LTNLESISKMLINNTITNTKGTITKEKHVNDFQKELKKITKLKEKHKTMYENDIIDIAELIEQTNKYR |
| | HREKEIKEIIHNIDKQDEKNEILKATLYNFNDAWAAATEPERKFLINSIFQNISIHAIGVHTRTKPRDIV |
| | ISSIY |
| 26 | MDKIKRVALYIRVSTEEQVLHGDSIRTQTEALEQYSKDNNFIIVDKYIDEGYSATNLKRPNLKRMIE |
| | DVKNNKIDLVMITKIDRLSRGVKNYYKIMETLEKHKCDWKTILEDYDSSTAAGRLHINIMLSVAEN |
| | EAAQTSERIKFVFQDKLKRGEVITGSVPFGYKIKDKHLVIKEDEASIVREAFDAYQDFSSLAKTIQHI |
| | NTKFSTKYMFKWMPKMLKNKIYIGIYEKGDLVVENYCEPIISREQFNFVQTLLKKNIRFSENKFKM |
| | NYLFSGMIVCGSCGRKMGGVHSRGGANRHYLYYRCPLSFATKLCDNKPYLNEKKVEAFLLENVK |
| | KELQKTILEHESNNKKRQKKNNNKNLRNKLEKQIEKLQDLYFDDLINKDTYKFKYKKLNDDLSEL |
| | NKAENEAESVEKDLKSMKIFLDTNFEDNYYDMNYSEKRTLWTSAIDRIEVQKNGELVIKFL |
| 27 | MSTDQEEQLSSYENQVNYYRDYISKHEDYELVDIYADEGISATNTKKRDAFNRLIQDCRAGKVDRI |
| | LVKSISRFARNTLDCIKYVRELKELGVGVTFEKENIDSLDSKGEVLLTILSSLAQDESRSISENATWGI |
| | RKKFERGEVRVNTTKFMGYDKDENGRLIINPGQAETVKFIYEKFLEGYSPESIAKYLNDNEIPGWTG |
| | KANWYPSAIQKMLQNEKYKGDALLQKTFTVDFLTKKRVQNDGQVNQYYVENSHEAIIDKDTWEL |
| | VQLELARRKDFREEHQLKAYIIQNDDNPFTTKVFCKACGSAFGRKNWTTSRGKRKVWQCNNRYR |
| | VKGQIGCQNNHIDEETLEKAVVMAVELLSENVDLLHGKWNKILEENRPLEKHYCTKLAEMINKPL |
| | WEFDSYEMCQVLDSITISEDGQISAKFLEGTEVDL |
| 28 | MKVPVWCYARISTLKQIDGFGIQRQINTINQFLQCVELDHRLPFTLDVDNVTQMVAEGKSAFRDKN |
| | WNEKTKLGQYRKLVMDGVISDSVLIVENIDRLTRLDPYMAIEIISGLVNRGTTILEIETGMTYSRYIP |
| | ESITVLVMQCNRANGESKRKSIMMQKSHANRYGKVSKVRPRWFDVVEIDGIKQYRPNETAKAIQR |
| | MYNDYINGIGAAHIVRTYGNTDNGKAWTLVTVLRALSDKRVADDARYPPIIDKKLYDSVQALKAA |
| | TNKKGNTHQKNMLNIFSGMSRCPVCNQSIIVKRNSHGNLFTVCLGKRTNKTCEARSISYFALERPLL |
| | TAIRDLDFSEVYKHEDKNVLTLRDQWIQNERDIAAFRERLSKASRYEKFVILDELETMNREQEELTI |
| | RLKSVDVPKDIQLTFDDDKLDLDTNYRIELNNRIKKLIQYINIVREDVTKSSYTIYCTIKYWTDVISH |
| | LVIIDVNIKRTGTGGTNTLTTTLRSVSSLNMDGTVSGNPDSDAWEYWKSFLDGTIGLVDYKK |
| 29 | MKKVFVYHRVSSDQQLDGSGIARQAELLEGYLERTGICAEMDDPAPVVLSDQGVSAFKGLNISEGE |
| | LGAWMEQVRNGMWDSSILVVESIDRFSRQNPFDVMGYINALMAHNVAIHDVMANIVISRSNSKDL |
| | PFVMMNAQQAYDESKYKSDRIRKGWAKKREQAFNKGTIVTNKRPQWIEVENDKYVLNHKAAVV |
| | KEIFALYQTGMGCPTIAKQLQTKEGEQYKFNRPWTGELVHKILTNRRVTGKIFISEIIRNHDDIENPV |
| | TQKKYDMDVYPVVINEEEFELVQELLKSRRPNAGRVTVKKDGQEEVLIKSNLFSGIARCTECGGPM |
| | YHNVVRAKRTPKKGDPKIEEYRYIRCLNERDGLCENKAMTYETVERFVVEHLLGMDLNTVIKEQE |
| | FNPEIEVIRIQIDQVKDHITNYENGIERRKSAGKAVSFEMREELDDAKLELEQLLARQASLATVQVD |
| | LPVLQDVNVTELYNVNNVDIRTRYENELNKIVSNIRLKRNGNFYTIDIIYKQNELKRHVLFIENKKK |
| | EQKLISEVIIENVDGAKFYYTPSFVISVKDGEIRFQQTKEDLTIIDYSLLLNYVDAVDRCDAVGVWM |
| | RNNMSFLFTK |
| 30 | MKVALYVRVSTLEQAEEGYSINEQKDKLKKYCEIKDWTIVKEYVDPGRSGSNINRPSMQQLIKDAD |
| | TGLYDAVLVYKLDRLSRSQKDTLYLIEDVFQKNNIHFISLSENFDTSTAFGKAMIGILSVFAQLEREQ |
| | IKERMSMGRIGRAKSGKIMEFNNPAFGYEIDGDNYKVDPLRAEIVKRIYKMYLSGTSINKIKETLNS |
| | EGHIGNKKNWSDTRIRYILSNPTYLGKIRYDGKTYDGKFSPIIDEETFNKTQNELKERQTATYKRFN |
| | MKLRPFQSKYMLSGLLRCGYCGATLFVNSYVYNGKRKLRYNCPSTYKSKQKTRTYKIMDPNCPFK |
| | LVYAKDLEPAVINEIKNLALNPQSIQKPVKKTPDIDVEAIQKELAKVRKQQQRLIDLYVISDDVNIDN |
| | ISKKSADLKLQEETLKKQLAPLEDPDDDDKIVAFNEILDQIKDIDSLDYDKQKFIVKKLIKKIDVWN |
| | DNKIKIHWNI |
| 31 | MNKVAIYVRVSTKGQAEEGYSIDEQIAMLTSYCSIHKWTVFDTYVDAGISGATIERPELSRLSRDAQ |
| | KKKFNTMIVYDLKRLGRSQRNNIAFIEDVLEKNGIGFISLTENFDTSTPLGKAMVGILSAFGQLDRD |
| | TIRERMMMGKIGRAKSGKPMMTSTIAFGYTYDKSTSTLNINPVEAIIVKTIFNEYLSGMSLTKLRDY |
| | LNKNDLLRNGRPWNYQGVSRLLRNPVYMGMIRFSGKVYQGNHEPIIDAETFETTQKELKRRQIAT |
| | YEFNKNTRPFRAKYMLSGIIRCACCGAPLHLVLRNKRKDGTRNMHYQCVNRFPRTTKGITVYNDG |
| | KKCNTEFYDKTNLEIYVLGQVRLLQLNKSKLDKMFETPVIINTEEIENQINSLNNKMRRLNDLYLND |
| | MVTLADLKAQTHTFLKQKELLENELENNPAIRQEEDRKKFKKLLGTKDITQLSYEEQTFTVKNLID |
| | KVFVKPSSIDIHWKI |
| 32 | MATKARVYSYLRFSDPKQAAGSSAARQLEYAKRWAAEHGMALDAALSMQDEGLSAYHQRHVTK |
| | GALGVFLAAIDEGRIPAGSVLIVEGLDRLSRAEPIQAQAQLAQIINAGITVVTASDGREYNRAGLKA |
| | QPMDLVYSLLVMIRAHEESDTKSKRVRAAIHRQCKGWKDGTWRGVIRNGKDPSWTRLDPETKAF |
| | QLVPERAEAVKLAIRMFRDGHGAVRIMRTLAEEGLQLTNGGNPAGQLYRILRNRALIGEKVLEIDG |
| | EEYRLAGYYPSLLSAEQFADLQQATEQRAKQKGTGEIPGLITGLRISYCGYCGSAMVAQNLMNRG |
| | RREDGGPQHGHRRLICVGNSQGMGCAVAGSCSVVPIEHAIMSYCADQMNLARLFEGGDRSEALAG |
| | KLAIARARVADTTAKVERITDAMLADDAGDAPAAFMRRARELEASLVEQQAEVDALEHELAAIAS |
| | SPTPAVAKAWADVQEGVKALDYNARTKARQLVADTFERISIYHRGTEPEQTRSWKGTIDLVLVAK |
| | RGSARILHVDRQTGEWRGGEEVRDLPDDPIQ |
| 33 | MKYAVYVRVSTDRDEQVSSVENQIDICRYWLEKNGYEWDPNAVYFDDGISGTAWLERHAMQLIL |
| | EKARRNELDTVVFKSIHRLARDLRDALEIKEILIGHGIRLVTIEENYDSLYEGGNDIKFEMFAMFAAQ |
| | LPKTLSVSISAAMQAKARRGEVIGKPGLGYDVIDKRLVINEKEAEVVREIFDLSKKGFGYKKIASILN |
| | DKGIYTKSGQLWSDTTIAKVLKNQKYKGDLVLNRYKTVKVDGRKKRIYTPKDRLTIIEDHYPAIVS |
| | KELWNEVNNNRVSQKKVKQNMRNEFRGMIFCNHCGGSITVKYSGKCSKKNKKEWVYLKCSNFL |
| | RFNQCVNFNPIYYDEIREMYRLKQKEKELEIHFNPKIHEKREAKSIEIKKDIKLLKAKKEKLIDLYVE |
| | GLIDKDVFSKRDLNIALNEIKEQELELLKLMDQNKRVNEEQQIKKAFSMLDEEKDMHEVFKILIKKIT |
| | LSKDKYVEIEYTFSL |
| 34 | MDTYAGAYDRQSRERENSSAASPATQRSANEDKAADLQREVERDGGRFRFVGHFSEAPGTSAFGT |
| | AERPEFERILNECRAGRLNMIIVYDVSRFSRLKVMDAIPIVSELLALGVTIVSTQEGVFRQGNVMDLI |
| | HLIMRLDASHKESSLKSAKILDTKNLQRELGGYVGGKAPYGFELVSETKEITRNGRMVNVVINKLA |
| | HSTTPLTGPFEFEPDVIRWWWREIKTHKHLPFKPGSQAAIHPGSITGLCKRMDADAVPTRGETIGKK |
| | TASSAWDPATVMRILRDPRIAGFAAEVIYKKKPDGTPTTKIEGYRIQRDPITLRPVELDCGPIIEPAE |
| | WYELQAWLDGRGRGKGLSRGQAILSAMDKLYCECGAVMTSKRGEESIKDSYRCRRRKVVDPSAP |
| | GQHEGTCNVSMAALDKFVAERIFNKIRHAEGDEETLALLWEAARRFGKLTEAPEKSGERANLVAE |
| | RADALNALEELYEDRAAGAYDGPVGRKHFRKQQAALTLRQQGAEERLAELEAAEAPKLPLDQWF |
| | PEDADADPTGPKSWWGRASVDDKRVFVGLFVDKIVVTKSTTGRGQGTPIEKRASITWAKPPTDDD |
| | EDDAQDGTEDVAA |
| 35 | MTKKVAIYTRVSTTNQAEEGFSIDEQIDRLTKYAEAMGWQVSDTYTDAGFSGAKLERPAMQRLIN |
| | DIENKAFDTVLVYKLDRLSRSVRDTLYLVKDVFTKNKIDFISLNESIDTSSAMGSLFLTILSAINEFER |
| | ENIKERMTMGKLGRAKSGKSMMWTKTAFGYYHNRKTGILEIVPLQATIVEQIFTDYLSGISLTKLR |
| | DKLNESGHIGKDIPWSYRTLRQTLDNPVYCGYIKFKDSLFEGMHKPIIPYETYLKVQKELEERQQQT |
| | YERNNNPRPFQAKYMLSGMARCGYCGAPLKIVLGHKRKDGSRTMKYHCANRFPRKTKGITVYND |
| | NKKCDSGTYDLSNLENTVIDNLIGFQENNDSLLKIINGNNQPILDTSSFKKQISQIDKKIQKNSDLYLN |
| | DFITMDELKDRTDSLQAEKKLLKAKISENKFNDSTDVFELVKTQLGSIPINELSYDNKKKIVNNLVS |
| | KVDVTADNVDIIFKFQLA |
| 36 | MRKVAIYSRVSTINQAEEGYSIQGQIEALTKYCEAMEWKIYKNYSDAGFSGGKLERPAITELIEDGK |
| | NNKFDTILVYKLDRLSRNVKDTLYLVKDVFTANNIHFVSLKENIDTSSAMGNLFLTLLSAIAEFERE |
| | QIKERMQFGVMNRAKSGKTTAWKTPPYGYRYNKDEKTLSVNELEAANVRQMFDMIISGCSIMSIT |
| | NYARDNFVGNTWTHVKVKRILENETYKGLVKYREQTFSGDHQAIIDEKTYNKAQIALAHRTDTKT |
| | NTRPFQGKYMLSHIAKCGYCGAPLKVCTGRAKNDGTRRQTYVCVNKTESLARRSVNNYNNQKIC |
| | NTGRYEKKHIEKYVIDVLYKLQHDKEYLKKIKKDDNIIDITPLKKEIEIIDKKINRLNDLYINDLIDLP |
| | KLKKDIEELNHLKDDYNKAIKLNYLDKKNEDSLGMLMDNLDIRKSSYDVQSRIVKQLIDRVEVTM |
| | DNIDIIFKF |
| 37 | MRKVAIYSRVSTINQAEEGYSIQGQIEALTKYCEAMEWKIYKNYSDAGFSGGKLERPAITELIEDGK |
| | NNKFDTILVYKLDRLSRNVKDTLYLVKDVFTANNIHFVSLKENIDTSSAMGNLFLTLLSAIAEFERE |
| | QIKERMQFGVMNRAKSGKTTAWKTPPYGYRYNKDEKTLSVNELEAANVRQMFDMIISGCSIMSIT |
| | NYARDNFVGNTWTHVKVKRILENETYKGLVKYREQTFSGDHQAIIDEKTYNKAQIALAHRTDTKT |
| | NTRPFQGKYMLSHIAKCGYCGAPLKVCTGRAKNDGTRRQTYVCVNKTESLARRSVNNYNNQKIC |
| | NTGRYEKKHIEKYVIDVLYKLQHDKEYLKKIKKDDNIIDITPLKKEIEIIDKKINRLNDLYINDLIDLP |
| | KLKKDIEELNHLKDDYNKAIKLNYLDKKNEDSLGMLMDNLDIRKSSYDVQSRIVKQLIDRVEVTM |
| | DNIDIIFKF |
| 38 | MKKAIAYMRFSSPGQMSGDSLNRQRRLIAEWLKVNSDYYLDTITYEDLGLSAFKGKHAQSGAFSE |
| | FLDAIEHGYILPGTTLLVESLDRLSREKVGEAIERLKLILNHGIDVITLCDNTVYNIDSLNEPYSLIKAI |
| | LIAQRANEESEIKSSRVKLSWKKKRQDALESGTIMTASCPRWLSLDDKRTAFVPDPDRVKTIELIFK |
| | LRMERRSLNAIAKYLNDHAVKNFSGKESAWGPSVIEKLLANKALIGICVPSYRARGKGISEIAGYYP |
| | RVISDDLFYAVQEIRLAPFGISNSSKNPMLINLLRTVMKCEACGNTMIVHAVSGSLHGYYVCPMRR |
| | LHRCDRPSIKRDLVDYNIINELLFNCSKIQPVENKKDANETLELKIIELQMKINNLIVALSVAPEVTAI |
| | AEKIRLLDKELRRASVSLKTLKSKGVNSFSDFYAIDLTSKNGRELCRTLAYKTFEKIIINTDNKTCDI |
| | YFMNGIVFKHYPLMKVISAQQAISALKYMVDGEIYF |
| 39 | MKAAIYIRVSTQEQVENYSIQAQTEKLTALCRSKDWDVYDIFIDGGYSGSNMNRPALNEMLSKLHE |
| | IDAVVVYRLDRLSRSQRDTITLIEEYFLKNNVEFVSLSETLDTSSPFGRAMIGILSVFAQLERETIRDR |
| | MVMGKIKRIEAGLPLTTAKGRTFGYDVIDTKLYINEEEAKQLQLIYDIFEEEQSITFLQKRLKKLGFK |
| | VRTYNRYNNWLTNDLYCGYVSYKDKVHVKGIHEPIISEEQFYRVQEIFTRMGKNPNMNRDSASLL |
| | NNLVVCSKCGLGFVHRRKDTMSRGKKYHYRYYSCKTYKHTHELEKCGNKIWRADKLEELIINRV |
| | NNYSFASRNVDKEDELDSLNEKLKIEHAKKKRLFDLYINGSYEVSELDSMMNDIDAQINYYESQIE |
| | ANEELKKNKKIQENLADLATVDFDSLEFREKQLYLKSLINKIYIDGEQVTIEWL |
| 40 | MTVGIYIRVSTEEQVKEGFSISAQKEKLKAYCTAQGWEDFKFYVDEGKSAKDMHRPLLQEMISHIK |
| | KGLIDTVLVYKLDRLTRSVVDLHNLLSIFDEFNCAFKSATEVYDTSSAMGRFFITIISSVAQFERENT |
| | SERVSFGMAEKVRQGEYIPLAPFGYTKGTDGKLIVNKIEKEIFLQVVEMVSTGYSLRQTCEYLTNIG |
| | LKTRRSNDVWKVSTLIWMLKNPAVYGAIKWNNEIYENTHEPLIDKATFNKVAKILSIRSKSTTSRRG |
| | HVHHIFKNRLICPACGKRLSGLRTKYINKNKETFYNNNYRCATCKEHRRPAVQISEQKIEKAFIDYIS |
| | NYTLNKANISSKKLDNNLRKQEMIQKEIISLQRKREKFQKAWAADLMNDDEFSKLMIDTKMEIDA |
| | AEDRKKEYDVSLFVSPEDIAKRNNILRELKINWTSLSPTEKTDFISMFIEGIEYVKDDENKAVITKISF |
| | L |
| 41 | MSPFIAPDVPEHLLDTVRVFLYARQSKGRSDGSDVSTEAQLAAGRALVASRNAQGGARWVVAGEF |
| | VDVGRSGWDPNVTRADFERMMGEVRAGEGDVVVVNELSRLTRKGAHDALEIDNELKKHGVRFM |
| | SVLEPFLDTSTPIGVAIFALIAALAKQDSDLKAERLKGAKDEIAALGGVHSSSAPFGMRAVRKKVDN |
| | LVISVLEPDEDNPDHVELVERMAKMSFEGVSDNAIATTEEKEKIPSPGMAERRATEKRLASVKARR |
| | LNGAEKPIMWRAQTVRWILNHPAIGGFAFERVKHGKAHINVIRRDPGGKPLTPHTGILSGSKWLEL |
| | QEKRSGKNLSDRKPGAEVEPTLLSGWRFLGCRICGGSMGQSQGGRKRNGDLAEGNYMCANPKGH |
| | GGLSVKRSELDEFVASKVWARLRTADMEDEHDQAWIAAAAERFALQHDLAGVADERREQQAHL |
| | DNVRRSIKDLQADRKPGLYVGREELETWRSTVLQYRSYEAECTTRLAELDEKMNGSTRVPSEWFS |
| | GEDPTAEGGIWASWDVYERREFLSFFLDSVMVDRGRHPETKKYIPLKDRVTLKWAELLKEEDEAS |
| | EATERELAAL |
| 42 | MAQPLRALVGARVSVVQGPQKVSHIAQQETGAKWVAEQGHTVVGSFKDLDVSATVSPFERPDLG |
| | PWLSPELEGEWDILVFSKIDRMFRSTRDCVKFAEWAEAHGKILVFAEDNMTLNYRDKDRSGSLES |
| | MMSELFIYIGSFFAQLELNRFKSRARDSHRVLRGMDRWASGVPPLGFRIVDHPSGKGKGLDTDPEG |
| | KAILEDMAAKLLDGWSFIRIAQDLNQRKVLTNMDKAKIAKGKPPHPNPWTVNTVIESLTSPRTQGI |
| | KMTKHGTRGGSKIGTTVLDAEGNPIRLAPPTFDPATWKQIQEAAARRQGNRRSKTYTANPMLGVG |
| | HCGACGASLAQQFTHRKLADGTEVTYRTYRCGRTPLNCNGISMRGDEADGLLEQLFLEQYGSQPV |
| | TEKVFVPGEDHSEELEQVRATIDRLRRESDAGLIATAEDERIYEERMKSLIDRRTRLEAQPRRASGW |
| | VTQETDKTNADEWTKASTPDERRRLLMKQGIRFELVRGKPDPEVRLFTPGEIPEGEPLPEPSPR |
| 43 | MYELKYAVYVRVSTDRDEQVSSIENQIDICRYWIEKNGYEWDENSIYKDEAVSGTAWLERHAMQL |
| | ILEKVRRKELDTVVFKSIHRLGRDLRDALEIKEILLGHGVRLVTIEEGYDSYYEGKNDLKFEMYAMF |
| | ASQLPKTVSVSVSAALAAKVRRGEYTGGIVPYGYKIVDQKYTINEEEAELVRKMYELYDNGLGYM |
| | KIADAINDMGVPSRTGKLWAYPSIRAIITNAAYKGDYIMQKYAEVKVDGRKKMIINPKEKWVVFE |
| | NHHPAIITRDLWDRVNNSKTDKKTKRRVAIKNELRGLACCAHCRTPLALQQRMYKNKEGETRYYC |
| | YLICGRYKRMGARGCVKHSGLQYSDLRLFVLQKLKEKENDLEKVFNLNDTDKHQEKQKKLRKEK |
| | KELEIKRERLLDLYLDGGPIDKETFTKRDKNELKIIKEKELEILKLDDVKTLVVEQQKVKEAFELLEK |
| | SEDLYSTFKKLITRIEVSQDGVINIVYRFEE |
| 44 | MLGRLRLSRSTEESTSIERQREIVTAWADSNGHTVVGWAEDVDVSGAIDPFDTPSLGVWLDERRGE |
| | WDILCAWKLDRLGRDAIRLNKLFLWCQEHGKTVTSCSEGIDLGTPVGRLIANVIAFLAEGEREAIRE |
| | RVASSKQKLREIGRWGGGKPPFGYMGVRNPDGQGHILVVDPVAKPVVRRIVEDILEGKPLTRLCTE |
| | LTEERYLTPAEYYATLKAGAPRQQAEEGEVTAKWRPTAVRNLLRSKALRGHAHHKGQTVRDDQG |
| | RAIQLAEPLVDADEWELLQETLDGIAADFSGRRVEGASPLSGVAVCMTCDKPLHHDRYLVKRPYG |
| | DYPYRYYRCRDRHGKNVPAETLEELVEDAFLQRVGDFPVRERVWVQGDTNWADLKEAVAAYDE |
| | LVQAAGRAKSATARERLQRQLDILDERIAELESAPNTEAHWEYQPTGGTYRDAWENSDADERREL |
| | LRRSGIVVAVHIDGVEGRRSKHNPGALHFDIRVPHELTQRLIAP |
| 45 | MAYAVYVRVSSDKDEQVSSVENQIDICRYWLENNGFEWDENAVYFDDGISGTAWLERHAIQLVLE |
| | KARKKEIDTVVFKSIHRLARDLKDALEIKEILLGHGVRLITIEEGYDSHYEGKNDMKFEMYAMFAS |
| | QLPKTLSVSITAALAAKVRRGGYTGGFVPYGYEIIDGKYAINEEEAALVKEIFELYAQGFGYIKIANT |
| | INDKGARTRKGAPWTFSTLSKMIKNPAYKGTYIMQKYGTVKVNGRKKKVINPKEKWVIFEDHHPA |
| | IISHELWEKVNNKDPNKFKKKRRVSTTNELRGITVCAHCGTAMSKRNSINVSKNGTETEYSYMICN |
| | WSRITARRECVRHVPIHYKDLRALVLSKLKEKEKELDKEFGSDENQLQVKLRKLKKDINDLKFKRE |
| | RLLDLYLEDERIDKDTFTIRNAKIEKEIGLKEMEIRKASNIEIQMKEKQEVRDAFALLEESKDLHSVF |
| | QKLIKRIEVAQDGAIDIYYRFEE |
| 46 | MDRDGDGLAVERQREDCLKICTDRGWEPTQYIDNDTSASRGRRPSYERMLSDIRSGHIDAVVAWD |
| | LDRLHRQPKELEQFIELADEKRLSLATVGGDADLSTDNGRLFARIKGAVAKAEVERKSARQKRAFL |
| | QMAQSGKGWGPRAFGYNGDHEKAKIVPKEADALRSGYKMLMSGETLYSIAKSWNDAGLKTPRG |
| | NLFTGTTVRRILQNPRYTATRTYRNETVGDGDWPAIVDETTWEAAHSILSDPSRHQPRQVRRYLLG |
| | GLLTCSECGNKMAVGVQHRKNGNVPIYRCKHVSCGRVTRRVERMDEWVKELVLRRMSSRHWVP |
| | GNQDNRELALELREELDAIKHRMDSLAVDFAEGELTSSQLRIANERLQVKLDEVESKLRRTNVKPL |
| | PDGILTANDRGRFYDEMSLDARRALIEALCDSIVVHPIGLKGMQATHAPLGHNIDVHWHKPSNG |
| 47 | MNKVAIYVRVSTTMQAEEGYSIDEQIDKLTSYCKIKDWTVYDIYKDGGFSGGNIERPAMERLISDA |
| | NRKRFDTVLVYKLDRLSRSQKDTLYLIEEIFGKNDISFLSLNESFDTSTPFGKAMIGILSVFAQLEREQ |
| | IKERMLLGKIGRAKSGKSMMVSKVSFGYTYDKLKGELIVNQAEALVVRKIFDEYLGGRSLIKLRDY |
| | LNSNGIYRGDKYWNYRGLLLILSNPVYIGMIRYRGEIYPGNHQPIIDTEVFNKTQEEIKKRQIEALEFS |
| | NNPRPFRAKYMLSGLAKCGYCGTPLKIILGYKRKDGSRSMRYQCINRFPRNTKGITIYNDNKKCDS |
| | GFYEKADIEEFVIAQIRGLQLNSYKLDNMFDKQPIIDVEGIEKQITSLDNKLKRLNDLYLNDMIELDD |
| | LKKQTQSLRKQKTMLEDELINNPAIMQDKNKNHFKEILGTKDITTLDYETQKSIVNNLVNKVFVKA |
| | GHIKIEWKIPFKKV |
| 48 | MNTINKVAIYVRVSTSVQAEEGYSIDEQIDKLKSYCQIKDWTVYDVYKDGGFSGGNINRPALEKMII |
| | DAKKKRFDTVLVYKLDRLSRSQKDTLYLIEDVFSKNDISFLSLQENFDTSTPFGKAMVGLLSVFAQL |
| | EREQIKERMQLGMIGRAKSGKPMMFTNVSFGYTYSPKTQQLTINQAEAVIVKQIFNEFLGGMSPLR |
| | LMAYLNENNILRNGKEWNYQGIQRILRNPVYIGKIKYNNVIYPGLHEPIIDEESYYKAQKLLDARQD |
| | EMRVKGKNRQFKAKYMLSGTAKCGYCGAPLRIKIGNKRLDGTRLKVYQCCNRYPRKYAVVTYN |
| | DNKKCNSGNYQKEDLEQYVIAEIRKLQLKPEKIDKLFNKVSKIDTVQINKQIASIDKKINRLNDLYL |
| | NDMIDIDKLKADAEKFKEQKRVLEKELDKDLKIQEQEKNKEDFKKTIGFKDVTKLDYEEQSFIVKS |
| | LIDKILVKKGLIKILWKI |
| 49 | MNVAIYCRVSTLEQKEHGYSIEEQERKLKQFCEINDWNVADVFVDAGFSGAKRDRPELQRMMNDI |
| | KRFDLVLVYKLDRLTRNVRDLLDLLEIFEQNNVAFRSATEVYDTSTAMGRLFVTLVGAMAEWERE |
| | TIRERTQMGKLAALKKGIMLTTPPFYYDRVDNKFVPNKYKEVVLFAYEEALKGKSAKSIARKLNN |
| | SDIPPPNNRKWEDRSITRALRSPFTRGHFEWGGVYLENNHEPIITEEMYEKVKDRLEERTNTKKIKH |
| | VSIFRSKLVCPTCDSKLTMNTHKVTLKDRVYYNKHYYCNNCKETPNLKPVYVRSEEVERVFYEYL |
| | QHQDLTQYDIVEDKEEKEIVIDINKIMQQRKRYHKLYANGLMNEDELAELIEETDIAIEEYKKQSEN |
| | EEVKQYDTEDIKQYKNLLLEMWEVSSDEEKAEFIQIAIKNIFIEYVLGKNDNKKKRRSLKIKDIEFY |
| 50 | MTVGIYIRVSTEEQARDGFSISAQREKLKAYCIAQDWDSFKFYVDEGVSAKDTNRPQLNMMLDHI |
| | KQGLISIVLVYRLDRLTRSVMDLYKLLDTFDEYNCAFKSATEVYDTSTAMGRMFITIVAALAQWER |
| | ENLGERVRMGQLEKARQGEYSAKAPFGFDKNKHSKLVVNDIESKVVLDMVKKIEEGYSIRQLANH |
| | LDGYAKPIRGYKWHIRTILDILSNHAMYGAIRWSNEIIENAHQGIISKDRFLKVQKLLSSRQNFKKRK |
| | TTSIFMFQMKLICPNCGNHLTCERVTYHRKKDNKDIEHNRYRCQACVLNKKKAFSSSEKKIEKAFL |
| | DYIDEYRFTKIPELKKEADETKILKKKLSKIERQREKFQKAWSNDLMTDEEFADRMKETKNTLGEIK |
| | EELNKLGLNQDKKIDNDTVKRIVNDIKNKWSLLSPLEKKQFMSLFIKNIQLKKINEKNIVVNITFY |
| 51 | MYRPDSLDVCIYLRKSRKDVEEERRALEEGSSYNALERHRKRLFAIAKAENHNIIDIFEEVASGESIQ |
| | ERPQMQQLLRKLEGNEIDGVLVIDLDRLGRGDMLDAGMIDRAFRYSSTKIITPTDVYDPDDESWEL |
| | VFGIKSLISRQELKSITRRLQNGRIDSVKEGKHIGKKPPYGYLKDENLRLYPDPEKAWIVKKIFELMC |
| | DGKGRQMIAAELDRLGIDPPVTKRGAWDSSTITSIIKNEVYTGVIVWGKFKHKKRNGKYTRHKNPQ |
| | EKWIMYENAHEPIISKELFDAANEAHSSRHKPAVITSKELTNPLAGILKCKLCGYTMLIQTRKDRPH |
| | NYLRCNNPACKGKQKQSVFNLVEEKLLYSLQQIVDEYQAQKVEEVEIDDSKLISFKEKAIISKEKEL |
| | KELQTQKGNLHDLLEQGIYTVEIFLERQKNLVERITSIENDVEVLQKEIEIEQVKEHNKTEFIPALKTV |
| | IESYHKTTNVELKNQLLKTILSTVTYYRHPDWKANEFEIQVYFKI |
| 52 | MITTNKVAIYVRVSTTNQAEEGYSIEEQKDKLKSYCNIKDWNVFNVYTDGGFSGSNTERPALEQLI |
| | KDAKKKKFDTVLVYKLDRLSRSQKDTLYLIEDIFLENNIDFVSLLENFDTSTPFGKAMVGILSVFAQ |
| | LEREQIKERMQLGKLGRAKAGKSMMWAKVAYGYTYHKGSGEMTINELEAIVVREIFNSYLEGMSI |
| | TKLRDKINDTYPKTPAWSYRIIRQILDNPVYCGYNQYKGEVYKGNHEPIISEEDFNKTQDELKIRQR |
| | TAAEKFNPRPFQAKYMLSGIAQCGYCKAPLKIIMGAVRKDGTRFIKYECYQRHPRTTRGVTTYNNN |
| | QKCHSSSYYKQDVEDYVLREISKLQNDKKAIDELFENTNMDTIDRESIKKQIEAISSKIKRLNDLYID |
| | DRITIDELRKKSTEFTLSKTFLEEKLENDPILKQQESKDNIKKILSCDDILTMDYDQQKIIVKGLINKV |
| | QVTADKVIIKWKI |
| 53 | MITTNKVAIYVRVSTTNQVEEGYSIDEQKDKLSSYCDIKDWNVYKVYTDGGFSGSNTDRPALESLI |
| | KDAKKRKFDTVLVYKLDRLSRSQKDTLHLIEDVFIKNGIEFLSLQENFDTSTPFGKAMIGLLSVFAQ |
| | LEREQIKERMQLGKLGRAKSGKSMMWAKTSYGYDYHKETGTVTINPAQALTIKFIFESYLRGRSIT |
| | KLRDDLNEKYPKHVPWSYRAVRTILDNPVYCGFNQYKGEIYPGNHEPIISKEEYDKTQSELKIRQRT |
| | AAENVNPRPFQAKYILSGIAQCGYCGAPLKIMLGVKRKDGSRLKKYECHQRHPRTLRGVTTYNDN |
| | KKCDSGFYYKDKLEAYVLKEISKLQDDADYLDKIFSGDNAETIDRESYKKQIEELSKKLSRLNDLYI |
| | DDRITLEELQSKSAEFISMRGTLETELENDPALRKNKRKADMRKLLNAEKVFSMDYESQKVLVRRL |
| | INKVKVTAEDIVINWKI |
| 54 | MKCVIYRRVSTDMQVEEGISLDMQKLRLEQYAKSQDWIVVNDYCDEGYSAKNTERPAFQQMIRD |
| | MKKKQFDIILVYRLDRFTRSVSDLHSILKIMDEYNVKFKSSTEIFDTTTATGRMFITLVATLAQWER |
| | ETTAERVRDSMHKKAELGLRNGAKAPMGYNLKKGNLYINHTEAEIVKYIFEMYKTKGVVSIVKSL |
| | NSRGVKTKQGKIFNYDAVRYIINNPIYIGKIRWGEDILTDIAQEDFETFINKDTWYTVQQIQDSRKVG |
| | KVRLQNFFVFSNVLKCARCGKHFLGNRQVRSHNRIAVGYRCSSRHHQGICDMPQVPENILEKEFLN |
| | LLEDAVVELDASDEKPVELSNLQEQYNRIQDKKARLKFLFIEGDIPKKEYKKDMLTLNQEENIIQKQ |
| | LANITDTVSSIEIKELLNQLKDEWNNLNNESKKAAVNAIISSITVDIIKPARAGKNPIPPVIKVMDFKL |
| | K |
| 55 | MKKAIAYMRFSSPGQMSGDSLNRQRRLIAEWLKVNSDYYLDTITYEDLGLSAFKGKHAQSGAFSE |
| | FLDAIEHGYILPGTTLLVESLDRLSREKVGEAIERLKLILNHGIDVITLCDNTVYNIDSLNDPYSLIKAI |
| | LIAQRANEESEIKSSRVKLSWKKKRQDALESGTIMTASCPRWLSLDDKRTAFVPDPDRVKTIELIFK |
| | LRMERRSLNAIAKYLNDHAVKNFSGKESAWGPSVIEKLLANKALIGICVPSYRARGKGISEIAGYYP |
| | RVISDDLFYAVQEIRLAPFGISNSSKNPMLINLLRTVMKCEACGNTMIVHAVSGSLHGYYVCPMRR |
| | LHRCDRPSIKRDLVDYNIINELLFNCSKIQPVENKKDANETLELKIIELQMKINNLIAALSVAPEVTAI |
| | AEKIRVLDKELRRASVSLKTLKSKAVSSLGDFHAIDLTSKNGRELCRTLAYKTFEKIIINTDNKTCDI |
| | YFMNGIVFKHYPLMKTISAQQAISTLKYMVDGEVYF |
| 56 | MKKAIAYMRFSSPGQMSGDSLNRQRRLITEWLKVNSDYYLDTVTYEDLGLSAFNGKHAQSGAFSE |
| | FLDAIEHGYILPGTTLLVESLDRLSREKVGEAIERLKLILNHGIDVITLCDNTVYNIDSLNEPYSLIKAI |
| | LIAQRANEESEIKSSRVKLSWKKKRQDALESGTIMTASCPRWLSLDDKRTAFVPDPDRVKTIELIFK |
| | LRMERRSLNAIAKYLNDHAVKNFSGKESAWGPSVIEKLLANKALIGICVPSYRARGKGISEIAGYYP |
| | RVISDDLFYAVQEIRLAPFGISNSSKNPMLINLLRTVMKCEACGNTMIVHAVSGSLHGYYVCPMRR |
| | LHRCDRPSIKRDLVDYNIINELLFNCSKIQPVENKKDANETLELKIIELQMKINNLIAALSVAPEVTAI |
| | AEKIRVLDKELRRASVSLKTLKSKAVSSLGDFHAIDLTSKNGRELCRTLAYKTFEKIIINTDNKTCDI |
| | YFMNGIVFKHYPLMKTISAQQAISTLKYMVDGEVYF |
| 57 | MKTAIYLRKSRADLEAEARGEGETLAKHRTTLLKIAKEMNLNVLAVREEIVSGESLVKRPEMLALL |
| | EEIEDNKYDVVLCMDMDRLGRGGMKEQGIILETFKRSNTKIMTPRKTYDLNDEWDEEYSEFEAFM |
| | ARKELKIITRRMQRGRIASVEAGNYLGTHAPYGYDIHRLNKRERTLTINSEEASVVRMIFDWYANE |
| | DMGANAIRSKLNDLGYKSKLGNEWNPYSILDILKNNVYIGKVTWQKRKEVKRPDAVKRSCARQD |
| | KSDWIIADGKHEPIIPESLFEQVQEKLNSRYHVPYNTNGIKNPLAGIIKCSKCGYSMVQRYPKNRKE |
| | TMDCKHRGCENKSSYTELIEKRLLEALKEWYINYKADIALKHKQDDKLKETQVIQMNEAALRKLEK |
| | ELVDVQKQKNNLHDLLERGVYTVDMFLERSNVVSDRITEITSTMENLKKEIKTEIKKEKVKKDTIPQ |
| | VEHVLDLYFKTDDPKKKNSLLKSVLEKAVYKKEKWQRLDDFELVLYPKLPQDGDI |
| 58 | MKTAIYLRKSRADLEAEARGEGETLAKHRTTLLKIAKEMNLNVLSVREEIVSGESLVKRPEMLALL |
| | EEIEDNKYDVVLCMDMDRLGRGGMKEQGIILETFKRSNTKIMTPRKTYDLNDEWDEEYSEFEAFM |
| | ARKELKIITRRMQRGRVASVEAGNYLGTHAPFGYDIHRLNKRERTLTINPEEASVVRMIFDWYANE |
| | DMGANAIRSKLNDLGYKSKLGNEWNPYSILDILKNNVYIGKVTWQKRKEVKRPDAVKRSCTRQD |
| | KSDWIIADGKHEPIIPESLIALQVQEKLNSRYHIPYNTNGIKNPLAGIIKCSKCGYSMVQRYPKNRKET |
| | MDCKHRGCENKSSYTELIEKRLLEALKEWYINYKADIALKHKQDDKLKETQVIQMNEAALRKLEKE |
| | LVDVQKQKNNLHDLLERGVYTVDMFLERSNVVSDRITEITSTMENLKKEIKTEIKKEKVKKDTIPQ |
| | VEHVLDLYFKTDDPKKKNSLLKSVLEKAVYKKEKWQRLDDFELVLYPKLPQDGDI |
| 59 | MKVAIYTRVSSAEQANEGYSIHEQKRKLISFCEVNDWNRYEVFSDPGVSGGSMKRPSLQKLFDRLE |
| | EFDLVLVYKLDRLTRNVRDLLEMLEVIALKNNIAFKSATEVFDTNSAIGKLFITMVGAMAEWERETI |
| | RERSLMGSHAAIRSGKYIRARPFCYDLIDDKLKPNQHAKYIRFMVDKLMIGKSASEVVRQLESKKK |
| | PPGITKWNRKMILNKSPNPVMRGHTKFGDLLIENTHEPIISEDEYLKLIDIIEKRTYKTKSKHKAIFRG |
| | VLECPRCQSKLHLSRSIKKYDNGKTREVRRYSCDKCHRDNTVKNISFNESEIERQFINTLLKKGTDN |
| | FKISVPKKKSYDIEDNKVKINEQRANYTRSWSLGYIKDEEYFMLMDETENLLKDIEEKAKSHTDEK |
| | LNEEQIRTVKNLLIKGFKIATLEDKEDLITSSVDVIKFEFIPKEFNKNKTLNTVKINEIQFKF |
| 60 | MKYAVYVRVSTDKDEQVSSIQNQIEICRYWIEKNGFEWDENSIYKDEAVSGTAWLERRAMQLILG |
| | KARKKELDTVVFKSIHRLGRDLRDALEIKEILLGHGVRLVTIEEGYDSYYEGENDLKFEMYAMFAS |
| | QLPKTLSVSISAALAAKVRRGEYTGGTVPYGYKIVDKKYVINQEEAEIVREMYELYDNGLGYLRIS |
| | NALNDVGKYKRSGKLWTYSAVKLIITNPMYKGDYVMGRSTEVKVDGRKKRIQEPREKWVVFENH |
| | HPAIIERSLWDKINNPKINKKIKRRVAVTNELRGIARCIHCGSPFVLHTYKYKNKEGEELNYGYLTC |
| | GTYKLTGGRGCVKHSGLRYERLRSLVLRKLKEKERDLEKVFKLNDKDKHQEKQKKLRKEKKELEI |
| | KRERLLDLYLDGGSIDKATFTKRDANFAKNIKEKELEILKLDDVKALIVEQQKVKDAFKLLEDSEN |
| | LYPVFKKLIARIDISQNGAVDIRYRFEE |
| 61 | MKYAVYVRVSTDKDEQVSSIQNQIEICRYWIEKNGFEWDENSIYKDEAVSGTAWLERRAMQLILG |
| | KARKKELDTVVFKSIHRLGRDLRDALEIKEILLGHGVRLVTIEEGYDSYYEGKNDLKFEMYAMFAS |
| | QLPKTLSVSISAALAAKVRRGEYTGGTVPYGYKIVDKKYVINQEEAEIVREMYELYDNGLGYLRIS |
| | NALNDVGKYKRSGKLWTYSAVKLIITNPMYKGDYVMGRSTEVKVDGRKKRIQEPREKWVVFENH |
| | HPAIIERPLWDKINNPKINKKIKRRVAVTNELRGIARCIHCGSPFVLHTYKYKNKEGEELNYGYLTC |
| | GTYKLTGGRGCVKHSRLRYERLRSLVLRKLKEKERDLEKVFKLNDKDKHQEKQKKLRKEKKELEI |
| | KRERLLDLYLDGGSIDKETFTKRDANFAKNIKEKELEILKLDDVKALIVEQQKVKDAFKLLEDSENL |
| | YPVFKKLIARIDISQNGAVDIRYRFEE |
| 62 | MMTTNKVAIYVRVSTTNQAEEGYSIDEQKDKLSSYCHIKDWSIYNIYTDGGFSGSNTERPALEQLV |
| | KDAKNKKFDTVLVYKLDRLSRSQKDTLYLIEDIFLENKIDFVSLLENFDTSTPFGKAMVGILSVFAQ |
| | LEREQIKERMQLGKLGRAKSGKSMMWAKTSYGYDYHKETGEMTINELEAIVIREIFQSYLGGRSIT |
| | KLRDDINQRYPKTPAWSYRIIRQILDNPVYCGYNQYKGKIYKGNHEPIISEEVYNKTQEELKIRQRT |
| | AAEKFNPRPFQAKYMLSGIAQCGYCQAPLTIIMGMVRKDGTRFIKYECKQRHPRKTTGVTVYNNN |
| | EKCHSGAYQKEEVEEYVLKEISKLQNDTSYLDEIFSTPETESIDRDSYQKQIDELTKKLSRLNDLYID |
| | DRITLEELQKKSAEFTTIRAFLEAELENDPSLKQQEKKEDMRKILGAEDIFLMDYEGQKTMVKGLIN |
| | KVQVTAEDISIKWKI |
| 63 | MNKVAIYVRVSTTMQAEEGYSIDEQIDKLKSYCKIKDWTVYDIYKDGGFSGGNIERPAMERLISDA |
| | KRKKFDTVLVYKLDRLSRSQKDTLFLIEEVFDKNDISFLSLNESFDTSTAFGKAMIGILSVFAQLERE |
| | QIKERMLLGKIGRAKTGKSMMFSKVSFGYTYDKLKDELVVNQAESIIVRKIFDAYLGGLSLNKLRD |
| | YLNNNGIYRGDKPWNYQGLRRILSNPVYIGMIRYREEIYPGNHKAIIDIDDYNKTQEEIKKRQIKALE |
| | FSNNPRPFRSKYMLSGIAKCGYCGTPLQIILGSKRKDGTRNMRYQCINRFPRNTKGVTIYNDGKKCE |
| | SGFYEKADIEEFVINEIRSLQINYNKLDAMFDRHPTVNSDDIKKQIITLDNKLKRLNDLYINNMIELD |
| | DLKKQTQSLRKQKTILEDELLNNPAITQEKNKKHFKEMLATKDITKLDYETQKNIVNNLINKVFVK |
| | SGYIKIEWKIPFKKA |
| 64 | MRKVYSYIRFSSTKQAFGDSHRRQSKAIQDWLASHPDHILDESLSFEDLGRSAFHGDHLKEGGALR |
| | AFLEAVKQGLIPPDSVLLVESLDRVSRQSISHAQETIRAILEQGITVVTLSDGETYNRQSLDDSLALIR |
| | MIILQERSHNESVIKSDRIKKVWSHKRQQFEQDGTKITGNCPGWLKLNSDGKSFSLIPHHVETIHRIF |
| | DEKLSGKSLHAIARDLNLENIPTITNKKVDTGWTPTRVRDLLLKESLIGVAYGVSDYFPPAISKEKFH |
| | AVQMISKRPISDVL |
| 65 | MKTAIYLRKSRADLEAEARGEGETLAKHRSTLLKIAKEMNLNVLAVREEIVSGESLVKRPEMLALL |
| | EEIEDNKYDAVLCMDMDRLGRGGMKEQGIILETFKRSNTKIMTPRKTYDLNDEWDEEYSEFEAFM |
| | ARKELKIITRRMQRGRIASVEAGNYLGTHAPFGYDIHRLNKRERTLTLNSEEASVVRMIFDWYANE |
| | DMGANAIRSKLNDLGYKSKLGNEWNPYSILDILKNNVYIGKVTWQKRKEVKHPDTVKRSCARQD |
| | KSDWIIADGKHEPIIPESLFEQVQEKLNSRYHIPYNTNGIKNPLAGIIKCAKCGYSMVQRYPKNRKET |
| | MDCKHRGCENKSSYTELIEKRLLEALKEWYINYKADFEKHKQDDKLKETQVIQMNEAALRKLEKE |
| | LVDVQKQKNNLHDLLERGVYTVDMFLERSNVVSVRITEITSTMENLKKEIKTEIKKEKVKKDTIPQ |
| | VEHVLDLYFKTDDPKKKNSLLKSVLEKAVYKKEKWQRLDDFELVLYPKLPQDGDI |
| 66 | MRIVNKIEAKTPQIPHRKRVAAYARVSMESERLQHSLSAQVSFYSSLIQSNPAWEYVGVYADNGIT |
| | GTKAEAREEFNRMIADCEAGKIDIVLTKSISRFARNTVDLLNTVRRLKELGVSVQ1ALKERIDSLTED |
| | GELMLTLLASFAQEEIRSLSDNVKWGTRKRFEKGIPNGRFQIYGYRWEGDHLVIHEEEAKIVRLIYD |
| | NYMNGLSAETTEKQLAEMGVKSYKGQHFGNTSIRQILGNITYTGNLLFQKEYVADPISKKSRINRG |
| | ELPQYFVENTHEAIIPMEVYQAVQAEKARRRELGALANWSINTSCFTSKIKCGRCGKSYQRSNRKG |
| | RKDPNANYTIWVCGTRRKTGNAYCQNKDIPEQMLKDACAEVMGLDTFDEIIFSEQIDHIEIPAPNEM |
| | IFYFKDGRIVPHHWESTMRKDCWTDERRAAKGRYVQEHQLGPNTSCFTSRIRCDSCGENYRRQRS |
| | RHKDGSFDSVWRCASGGKCQSPSIKEDALKNLCADAMGLEEFSETVFREQIVCIHITAPYQLSIRFF |
| | DGHTIALTAWENKRKMPRHTEERKQHMREVMIQRWREKRGESNDNTCDDKPIHGNADQ |
| 67 | MKGESELDKKAAIYIRVSTQEQATEGYSIQAQTDRLTKYVEAKDFILYKKYIDAGYSASKLERPAM |
| | QELIQDVKSKKVDVVIVYKLDRLSRSQKDTMYLIEDIFRPNDVELISMQESFDTSTAFGSATVGMLS |
| | VFAQLERKSISERMITGRVERAKKGFYHTGGQDRPPAGYQFNSDNQLIINEYEAAAIKDLFRLYND |
| | GLGKSSISEYLKKNYPGKNKWLPSSIDRMLKNSLYIGKVKFSGVEYDGIHEPIIDEVTFYKTQKEIAR |
| | RKQSNTKRYNYVALLGGLCECGICGAKMANRRAVGRKGKVYRYYRCYSKKGSPKHMMKTDGCS |
| | SKAQQQFIIDEAVINNLKNIDVEAELKRRSAPQTNTSLISSQIESIDKQINKLIDLFQVDSMPLDVISEK |
| | IDKLNKEKQSMEKLLERKNRLDKTELQHRFDILKSFDWDNSSIESKRAVIEMLVQKVIIHDNSIEIILV |
| | E |
| 68 | MKGESELDKKAAIYIRVSTQEQATEGYSIQAQTDRLTKYVEAKDFILYKKYIDAGYSASKLERPAM |
| | QELIQDVQSKKVDVVIVYKLDRLSRSQKDTMYLIEDIFRPNDVELISMQESFDTSTAFGSATVGMLS |
| | VFAQLERKSISERMITGRVERAKKGFYHTGGQDRPPAGYQFNSDNQLIINEYEAAAIKDLFRLYND |
| | GLGKSSISEYLKKNYPGKNKWLPSSIDRMLKNSLYIGKVKFSGAEYDGIHEPIIDEVTFYKTQKEIAR |
| | RKQSNTKRYNYVALLGGLCECGICGAKMANRRAVGRKGKVYRYYRCYSKKGSPKHMMKTDGCS |
| | SKAQQQFIIDEAVINNLKNIDVEAELKRRSAPQTNTSLISSQIESIDKQINKLIDLFQVDSMPLDVISEK |
| | IDKLNKEKQSMEKLLERKNKLDKTELQHRFDVLKSFDWDNSSIESKRVVIEMLVQKVIIHDNSIEIIL |
| | VE |
| 69 | MDKKAAIYIRVSTQEQATEGYSIQAQTDRLTKYVEAKDFILYKKYIDAGYSASKLERPAMQELIQD |
| | VQSKKVDVVIVYKLDRLSRSQKDTMYLIEDIFRPNDVELISMQESFDTSTAFGSATVGMLSVFAQLE |
| | RKSISERMITGRVERAKKGFYHTGGQDRPPAGYQFNSDSQLIINEYEAAAIKDLFRLYNDGLGKSSIS |
| | EYLKKNYPGKNKWLPSSIDRMLKNSLYIGKVKFSGTEYDGIHEPIIDEVTFYKTQKEIARRKQTNTK |
| | RYNYVALLGGLCECGICGAKMANRRAVGRKGKVYRYYRCYSKKGSPKHMMKTDGCSSKAQQQF |
| | IIDEAVINNLKNIDVEAELKRRSAPQTNTSLISSQIESIDKQINKLIDLFQVDSMPLDVISEKIDKLNKE |
| | KQSMEKLLERKNKLDKTELQHRFDVLKSFDWDNSSIESKRVVIEMLVQKVIIHDNSIEIILVE |
| 70 | MKGESELDKKAAIYIRVSTQEQATEGYSIQAQTDRLIKYVEAKDFILYNKYIDAGYSASKLERPAM |
| | QELIQDVQSKKVDVVIVYKLDRLSRSQKDTMYLIEDIFRPNDVELISMQESFDTSTAFGSATVGMLS |
| | VFAQLERKSISERMITGRVERAKKGFYHTGGQDRPPAGYQFNSDNQLIINEYEAAAIKDLFRLYND |
| | GLGKSSISEYLKKNYPGKNKWLPSSIDRMLKNSLYIGKVKFSGAEYDGIHEPIIDEVTFYKTQKEIAR |
| | RKQTNTKRYNYVALLGGLCECGICGAKMANRRAVGRKGKVYRYYRCYSKKGSPKHMMKTDGCS |
| | SKAQQQFIIDEAVINNLKNIDVEAELKRRSAPQTNTSLISSQIESIDKQINKLIDLFQVDSMPLDVISEK |
| | IDKLNKEKQSMEKLLERKNRLDKTELQHRFDVLKSFDWDNSSIESKRVVIEMLVQKVIIHDNSIEIIL |
| | VE |
| 71 | MKGESELDKKAAIYIRVSTQEQATEGYSIQAQTDRLIKYVEAKDFILYKKYIDAGYSASKLERPAM |
| | QELIQDVQSKKVDVVIVYKLDRLSRSQKDTMYLIEDIFRPNDVELISMQESFDTSTAFGSATVGMLS |
| | VFAQLERKSISERMITGRVERAKKGFYHTGGQDRPPAGYQFNSDNQLIINEYEAAAIKDLFRLYND |
| | GLGKSSISEYLKKNYPGKNKWLPSSIDRMLKNSLYIGKVKFSGAEYDGIHEPIIDEVTFYKTQKEIAR |
| | RKQTNTKRYNYVALLGGLCECGICGAKMANRRAVGRKGKVYRYYRCYSKKGSPKHMMKTDGCS |
| | SKAQQQFIIDEAVINNLKNIDVEAELKRRSAPQTNTSLISSQIESIDKQINKLIDLFQVDSMPLDVISEK |
| | IDKLNKEKQSMEKLLERKNKLDKTELQHRFDVLKSFDWDNSSIESKRVVIEMLVQKVIIHDNSIEIIL |
| | VE |
| 72 | MNYERSYLRSCQVSTLEQKEHGYSIEEQERKLKSFCEINDWSVSDVFIDAGFSGAKRERPELQRMM |
| | NDIKRFDLVLVYKLDRLTRNVRDLLDLLEIFEQNNVAFRSATEVYDTSTAMGRLFVTLVGAMAEW |
| | ERETIRERVMMGKRAAIKQGMILTPPPFYYDRVDNTYIPNDYKKVVLWAYDEVMKGVSSKGIARK |
| | LNDSDIPPPNGKRWEDRTITRALRSPITRGHYTWGDVFIENSHEPIITEEMYQQIKERLEERINTKIVS |
| | HVSVFRGKFICPRCGGTLTLNTVTRKRKKGYVTYKTYYCNTCKAKKESFGFSENEALRVFRDYLSE |
| | LDLDKYKVKTKQNDDVVTIDIDKIMEQRKRYHKLYAKGLMQEEELFELIKETDETIAEYEKQKELV |
| | PRKSLDIDKIKKFKNALLESWEIFSLEDKADFIKMAIKSIDIEYVKLKNRHSIEIKDIEFY |
| 73 | MNYERSYLRSCQVSTLEQKEHGYSIEEQERKLKSFCEINDWSISDVFIDAGFSGAKRERPELQRMM |
| | KDIKRFDLVLVYKLDRLTRNVRDLLDLLEIFEQNNVAFRSATEVYDTSTAMGRLFVTLVGAMAEW |
| | ERETIRERVMMGKRAAIKQGMILTPPPFYYDRVDNTYVPNNYKKVVLWAYDEVLKGVSSKGIARK |
| | LNDSDIPPPNGKRWEDRTITRALRSPITRGHYTWGDVFIENSHEPIITEEMYQQIKERLEERINTKIVS |
| | HVSVFRGKFICPKCGGTLTMNTATRKRKKGYVTYKTYYCNTCKTKKQSFGFSENEALRVFRDYLS |
| | KLDLEKYEIKTKQKDDVVTIDIDKIMEQRKRYHKLYAKGLMQEEELFELIKETDETIAEYEKQKELA |
| | PSKTLDVAKIKKFKNALLESWKIFSLEDKADFIKMAIKSIDIDYVKLKNRHSIKINDIEFY |
| 74 | MNYERRYIRSCQVSTLEQKEHGYSIEEQERKLKSFCEINDWSVSDVFIDAGFSGAKRDRPELQRMM |
| | NDIKRFDLVLVYKLDRLTRNVRDLLDLLEIFEQNNVAFRSATEVYDTSTAMGRLFVTLVGAMAEW |
| | ERETIRERVMMGKRAAIKQGMILTPPPFYYDRVDNTYIPNDYKKVVLWAYDEVMKGNSSKAIARK |
| | LNDSDIPPPNGKRWEDRTITRALRNPITRGHYTWGDVFIENSHEPIITEEMYQQIKERLEERINTKIVS |
| | HVSVFRGKFICPRCGGTLTMNTATRKRKKGYVTYKTYYCNTCKTRKQSFGFSENEALRVFRDYLS |
| | KLDLDKYEVKTKQKDDVVTIDIDKIMEQRKRYHKLYAKGLMQEEELFELIKETDETIAEYEKQKEL |
| | VPRKILDIDKIKSFKNVLLESWNIFSLEDKADFIKMAIKSIEIEYVELKNRHSIEIKEIEFY |
| 75 | MKTAIYLRKSRADLEAEARGEGETLAKHRTTLLKIAKELNLDVLSVREEIVSGESLVKRPEMLALLE |
| | EIEDNKYDVVLCMDMDRLGRGGMKEQGIILETFKRSNTKIMTPRKTYDLNDEWDEEYSEFEAFMA |
| | RKELKIITRRMQRGRIASVEAGNYLGTHAPYGYDILRLNKRERTLTINSEEASVVRMIFEWYANED |
| | MGASVITNKLNQLGYKSKLGNDWNPYSVLDMLKNNIYIGKVTWQKRKEVKRPDATKRSCARQDK |
| | SEWIIADGKHDPIISKSLFEKAQEKLNTRYHVPYNTNGLKNPLAGIIRCGKCGYSMVQRYPKNRKKT |
| | MDCKHRGCENKSSYTELIERRLLEALKEWYINYKADFAKNNQDSLSKEKQVIKINQAALRKLEKEL |
| | LDVQKQKNNLHDLLERGVYTVDMFLERSNVVSDRMNEITEMMENLQKEINTEIKKERVKKDTIPQ |
| | VEHVLDLYFKTDDPKKKNSLLKSVLEKAVYTKEKWQRLDDFKLVLYPRLPKDGDK |
| 76 | MKIAIYSRKSVSTDKGESIKNQIEICKEYFLRRNTNIEFEIFEDEGFSGGNTNRPAFKFMMSKIKMFD |
| | VVACYKIDRIARNIVDFVNVYDELNKLGIKLISVTEGFDPSTPLGKLIMMILASFAEMERENIRQRVK |
| | DNMKELAKAGRWTGGNVPFGFISQRIEEGGKKATYLKLDENKKQLIKEIFDMYISANSMHKVQKQ |
| | LYIIHNIKWSLSTIKNILTSPVYVKADKDVVKYLNNFGKVFGEPNGANGMITYNRRPYTNGKHRWN |
| | DKGMFYSISRHEGIIDSSTWLKVQSIQEKTKVAPRPKNSKVSYLTGILKCAKCGSPMTISYNHKNKD |
| | GSITYVYLCTGRKTYGKEYCTCKQVKQTIMDKEIENALNSYIQLNIEEFKKVIGSPNDTENFNKNILC |
| | IEKKIETNKVKINNLVDKISILSNTASAPLLSKIEELTKLNEDLKKELLFIQQEHINSTFVSPEEKYERL |
| | KQFSYTLNTNDIDLKRELLSFSVQEIKWDSDEKCIDIII |
| 77 | MHKAAAYARYSSDNQREESIEAQLRAIREYCQKNNIQLVKIYTDEAKSATTDDRPGFLQMIQDSSM |
| | GLFSAVIVHKLDRFSRDRYDSAFYKRQLKKNGVRLISVLENLDDSPESIILESVLEGMAEYYSRNLA |
| | REVMKGMRETALQCKHTGGKPPLGYDVAEDKTYIVNEQEAQAVRLIFEMYASGKGYSDIMYALN |
| | KEGYRTQTGRPFGKNSIHDILRNEKYRGVFIFNRTERKINGKRNHHRNKDDSEIIRIEGGMPRIIDDE |
| | TWERVQERMSKNKKGANSAKENYLLAGLIYCGKCGGAMTGNRHRCGRNKTLYVTYECSTRKRT |
| | KECDMKAINKDYIENLVIEHLEKNVFAPEAIERLVAKISEYAASQVEEINRDIKTFTDQLAGIQTEIN |
| | NIVNAIAAGMFHPSMKEKMDELETKKANLLLKLEEAKFVFCK |
| 78 | MKAAIYIRVSTQEQVENYSIQAQTEKLTALCRSKDWDVYDIFIDGGYSGSNMNRPALNEMLSKLHE |
| | IDAVVVYRLDRLSRSQRDTITLIEEYFLKNNVEFVSLSETLDTSSPFGRAMIGILSVFAQLERETIRDR |
| | MVMGKIKRIEAGLPLTTAKGRTFGYDVIDTKLYINEEEAKQLQMIYDIFEEEKSITSLQKRLKKLGF |
| | KVKSYSSYNNWLTNDLYCGYVSYADKVHTKGVHEPIISEEQFYRVQEIFSRMGKNPNMNRDSASL |
| | LNNLVVCSKCGLGFVHRRKDTVSRGKKYHYRYYSCKTYKHTHELEKCGNKIWRADKLEELIIDRV |
| | NNYSFASRNVDKEDELDNLNEKLKTEHTKKKRLFDLYISGSYEVSELDAMMADIDAQINYYEAQIE |
| | ANEELKKNKKIQENLADLATVDFDSLEFREKQLYLKSLINKIYIDGEQVTIEWL |
| 79 | MKTIHKLARPQLPEPPKLKVAAYARASTSSNEQLASLQTQITHYENHIQNNDQWEYVGVYYDEGT |
| | SGTKVEKRDGLHRLIKDAELGKIDLILTKSISRFSRNTVDCLNLVRKLTDIGVTIFFEKENINTGDMES |
| | ELLLSILSSLAESESYSHSENMKWANRKRMAKGIFKTVPPYGYQRKGADFYLIPDEAKVIEQIFKWA |
| | LEGVSAYQVAKRLNEKNIFTRKGSKWQDSGINNILHNIVYTGTMIHQRYFNDDQFRKKKNNGELP |
| | MYRIDNNHPPIISWEDYERVQELITLRANAKGTSKGSQKYSQRYVFTKRIICDKCGCNYKRVHIAGK |
| | GNTKVVKWSCTGHLKNKDGCDALPITDESLKTAYLTMLNKLILGHTIVLEPLINTPVEGKASKQEL |
| | EKLSIEITKIDEKLEVLASLNASGVVSTKTALEEQGRLQMELNKLQEKQHKIMESVNGTSTQRIQLE |
| | QLHQFTKRSEMLTEWDEDLFLRFAELIVVYSRQEVSFELKCGLLLKERLEA |
| 80 | MPIQKSRRLSKVAGKKVTVIPMKPRQWAAENTEEKPKLKVAAYCRVSTEMEEQASSYEAQVQHY |
| | TDYIQRNPDWELAGIFADEGISGTGTKKRDGFNRMIEACQKGDVEYIITKSISRFARNTVDCLQYIRQ |
| | LKDLHIAVFFEKENINTMDAKGEVLLTIMASLAQQESQSLSQNTKMGVQYRFQQGQLRINHNHFLG |
| | YTKDEDGNLVIEPKEAEVIKRIFREYLEGSSLQEIANGLMSDGILTGGKRKLWRGEGVRLILRNEKY |
| | MGDALLQKTYTTDFLTKKRVKNDGSYAQQYYVENSHPAIIPRDIFMQVQQELDRRKSMKNKHSQ |
| | CFSGKYALSGITVCGDCGNAYRRVHWKNRGTVWRCKSRVDKREHNCSGRTIYEKDLHEAIIKAIN |
| | ETVVDREDFLQQLSENINSVLTDGLTGRLEELDSKLKELESEIISMAIGGQGYDELASQIFSLRDERD |
| | AVAKQIAANTNLQQRVDEMVVFVKEHDVINEYSEVLVRRLIEKVTIFEKNIVVDFKSGVRVTVEI |
| 81 | MKAAIYIRVSTQEQIENYSIQAQTEKLTALCRSKDWDVYDIFIDGGYSGSNMNRPALNEMLSKLHEI |
| | DAVVVYRLDRLSRSQRDTITLIEEYFLKNNVEFVSLSETLDTSSPFGRAMIGILSVFAQLERETIRDR |
| | MVMGKIKRIEAGLPLTTAKGRTFGYDVIDTKLYINEEEAKQLRLIYDIFEEEQSITFLQKRLKKLGFK |
| | VRTYNRYNNWLTNDLYCGYVSYKDKVHVKGIHEPIISEEQFYRVQEIFSRMGKNPNMNKESASLL |
| | NNLVVCSKCGLGFVHRRKDTVSRGKKYHYRYYSCKTYKHTHELEKCGNKIWRADKLEELIIDRVN |
| | NYSFASRNIDKEDELDSLNEKLKIEHTKKKRLFDLYISGSYEVSELDAMMSDIDAQINYYEAQIEAN |
| | EELKKNKKIQENLADLATVDFNSLEFREKQLYLKSLINKIYIDDEQVTIEWL |
| 82 | MRYTTPVRAAVYLRISEDRSGEQLGVARQREDCLKLCGQRKWVPVEYLDNDVSASTGKRRPAYE |
| | QMLADITAGKIAAVVAWDLDRLHRRPIELEAFMSLADEKRLALATVAGDVDLATPQGRLVARLKG |
| | SVAAHETEHKKARQRRAARQKAERGHPNWSKAFGYLPGPNGPEPDPRTAPLVKQAYADILAGASL |
| | GDVCRQWNDAGAFTITGRPWTTTTLSKFLRKPRNAGLRAYKGARYGPVDRDAIVGKAQWSPLVD |
| | EATFWAAQAVLDAPGRAPGRKSVRRHLLTGLAGCGKCGNHLAGSYRTDGQVVYVCKACHGVAI |
| | LADNIEPILYHIVAERLAMPDAVDLLRREIHDAAEAETIRLELETLYGELDRLAVERAEGLLTARQV |
| | KISTDIVNAKITKLQARQQDQERLRVFDGIPLGTPQVAGMIAELSPDRFRAVLDVLAEVVVQPVGKS |
| | GRIFNPERVQVNWR |
| 83 | MEKVAIYIRVSKKEQTRDKGSDSSLNLQLKKCLDYCKEKGYEVLKVYQDIESGRIDDRKEFNELFE |
| | AISKKIYTKIVFWEISRIARKISTGMKFFEELELYKITFDSISQPYLKDFMTLSIFLAWGTEDLKQMSL |
| | RIKSNLEEKTKAGYFVHGRPATGYIRGENKMIIPDPEKAPYILSIFETYAKNFNLTETARIFNKTRMDI |
| | VDIIDNKIYIGYVPFRKYIQELNQKKRIQVSKKDIKWYKGLHEPIVPLELFEFCQSIREKNIKSRAAYG |
| | DYKPHLLFSSMIYCECGDKMYQQKRNRTYKDNTNYVYYSYSCKNRKHKKSFSARIMDKTIKEMIL |
| | NSKELEDLNNYNSNDIEKNEKKLLKLEKNLKVLENERERIINLFQKSYISEDELENRFKDLNARIKIA |
| | KEKKIEFEKNLNIPKNNDIKLLEKLKFIIENYDEEDVIETRKILKMLIKEIRVISFYPLKISILFY |
| 84 | MQTLQAKIAVKYSRVSTNKQDLRGSKDGQEAEIDKFAIANNFTIISSFTDTDHGDIAKRKGLSSMKE |
| | YLRLNQAVKYVLVYHSDRFTRSFQDGMRDLFFLEDLGIKLISVLEGEIVADGTFNSLPSLVRLIGAQ |
| | EDKAKIIKKTTDASYKYAKTNRYLGGNILPWFKLESGYVYGKKCKVIVKNEATWEYYRGFFLAMI |
| | KYKNILRAAKEYNLNSFTVAEWLTKPELIGYRTYGKKGKIDQYHNKGRRKNYQTTEEKIFPAILTE |
| | EEFLVLNEMRKYNRAKYNKDIYTYLYSNLSYHSCGGKLEGERIKKKDSFVYYYKCNCCKKRFNQK |
| | KIETAIAENILNNPGLQIINDINFRLADIYDEIKNINNMIEEENSSEKRILSLVSKNVVGVEAAEEELLKI |
| | KKQKNFLKKLLEEKIKLIEEENKKEITEDHISLLKNLLEYSQEDDDDFRGKLKEIINLIVRKIEVSSLD |
| | KINIIF |
| 85 | MEKVAIYIRVSKKEQTRDKGSDSSLNLQLKKCLDYCKEKGYEVLKVYQDIESGRIDDRKEFNELFE |
| | AISKKIYTKIVFWEISRIARKISTGMKFFEELELYKITFDSISQPYLKDFMTLSIFLAWGTEDLKQMSL |
| | RIKSNLEEKTKAGYFVHGRPATGYIRGENKMIIPDPEKAPYILSIFETYAKNFNLTETARIFNKTRMDI |
| | VDIIDNKIYIGYVPLRKYVKELNQKNRTQVSKKDIKWYKGLHEPIVPLELFEFCQSIREKNIKSRVVY |
| | GDYKPYLLFSSMIYCECGDKMYQQKRNRSYKDNTKYAYYSYSCKNRKHRKSFSAKIMDKTIKEMI |
| | LNSKELEDLNNYNSNDIEKNEKKLLKLEKNLKVLENERERIINLFQKSYISEDELENRFKDLNARIKI |
| | AKEKKIEFEKNLNIPKNNDIKLLEKLKFIIENYDEEDVIETRKILKMLIKEIRVISFYPLKISILFY |
| 86 | MAQRKVTAIPATITKYTAVPIGSKRKRRVAGYARVSTDHEDQVTSYEAQVDYYTNYIKGRDDWEF |
| | VAIYTDEGISATNTKRREGFKAMVADALAGKIDLIVTKSVSRFARNTVDSLTTVRTLKEKGVEIYFE |
| | KENIWTLDAKGELLITIMSSLAQEESRSISENTTWGQRKRFADGKASVAYKRFLGYDRGPNGGFVV |
| | NQEQAKTVKLIYKLFLDGLTCHAIAKELTERKLPTPGGKAVWSQSTVRSILTNEKYKGDALLQKEF |
| | TVDFLQKKTKKNEGEVPQYYVEGNHEAIIDPATFDYVQAEMARRMKDKHRYSGVSMFSSKIKCGE |
| | CGCWYGSKVWHSTDKYRRVIYQCNHKYKGGKTCGTPHVTEKQVKGAFVRATNILLSERDELTAN |
| | TRMVIVMLCDSTELEKRQAELKEELEVVVGLVERCVAENARTALDQDEYTERYNGLVSRYETVKT |
| | RFDEVTQAIADKADRKKLLEQFLHTVETQEPVTQFDERLWSSLVDFVTVYSEKDIRVTFKDGTEIQ |
| | V |
| 87 | MPNLRKIEAAVPAIREKKKVAAYARVSMQSERMLHSLSAQVSYYSGLIQKNPDWEYAGVYADDFI |
| | SGTNTVKRDEFKRMLADCEAGKIDIILTKSISRFARNTVDLLETVRHLKDLGVEVQFEKERIRSMDG |
| | DGELMLTILASFAQEESRSISDNVKWGIRKRMQNGIPNGHFRIYGYRWEGDELVIVPEEAEVVKRIF |
| | RNFLDGKSRLETERELAAEGITTRDGCRWVDSNIKVVLTNVTYTGNLLLQKEFISDPISKQRKKNRG |
| | ELPQYYVEDTHPAIIDKATFDFVQEEMARRRELGALANKSLNTSCFTGKIKCPYCGQSYMHNKRTD |
| | RGDMEFWNCGSKKKKKKGTGCPVGGTINHKNMVKVCTEVLGLDEFDEAIFLEKVDHIDVPERYTL |
| | EFHMADGNVVTKDCLNTGHRDCWTPERRAEVSMKRRKNGTNPIGASCFTGKIKCVSCGCNFRKA |
| | TRNCKDGSKVSHWRCAEHNGCDSPSLREDLLEQMAAEVLGLDAFDAAAFREKIDRVEVLSSSELR |
| | FCFKDGRTVSRNWQPPERVGRPWTEEQRAKFKESIKGAYTPERRRQMSEHMKQLRKERGDKWRR |
| | EK |
| 88 | MTVGIYIRVSTEEQAREGFSISAQREKLKAYCISQDWQDYKFYVDEGKSAKDTNRPYLKLMLDHIQ |
| | QGLINVVLVYRLDRLTRSVKDLYKLLDLFDKNNCIFRSATEVYDTGSATGRLFITLVAAMAQWERE |
| | NLGERVTMGQVEKARQGQYSAPAPFGFKKQDETLVKDKKQGYILMDMIDKVKKGWSIRQIAKYL |
| | DQSYLPIRGYKWHIATILSILHNPALYGALRWKDELNETSHEGYLTKEEFEELQNILYSRQNFRKRQI |
| | ESAHIFQMKLVCPQCGNRLGCERSVYFRKKDQKNVESLHYRCQSCALNERPSISVSEKKLEKALLL |
| | FMKNVKFDLEPVVKEEKNETTEIQNAIVKIERQREKFQKAWASDLMTDEEFTARMSETRKAHENFT |
| | KRLSEIQRATPVPIDIKKAKKLVNEFKINWAYLNTEEKREFVQSFIEKIEFTKKDQNPHILNVSFY |
| 89 | MLKEVRCAIYTRKSNEDGLEQKFNSLDAQRVVCEKYIKSREGWVALAKKYDDGGFSGSNLNRPAI |
| | KELFEDVKVGEVDCVVVYTLDRLSRETKDCIEVTSFFRRHRISFVAVTQIFDNNTPMGKFVQTVLSG |
| | AAQLEREMIVERVKNKIATSKEQGLWMGGNPPLGYDVKEKELIINEKEAKIIKHIFERYMELKSMA |
| | ELARELNREGYRTKAKSDIFKKATVRRIITNPIYMGKIRHYEKQYKGKHEAIIEEEKWQKAQELISN |
| | QPYRKAKYEEALLKGIIKCKSCDVNMTLTYSKKENKRYRYYVCNNHLRGKNCESVNRTIVAGEIE |
| | KEVMKRAECLYGDGENLSFREQKEAMKKLIKGVMVKEDGIEVCSESEEKFIPMKKKGNKCIVIEPE |
| | GKTNNALLKAVVRAHSWKRQLEEGKYRSVKELSKKINVGTRRIQQILRLNYLAPKIKEDIVNGRQP |
| | RGLKLVDLKEIPMLWSEQREKFYGLDL |
| 90 | MKGESELDKKAAIYIRVSTQEQATEGYSIQAQTDRLTKYVEAKDFILYKKYIDAGYSASKLERPAM |
| | QELIQDVQSKKVDVVIVYKLDRLSRSQKDTMYLIEDIFRPNDVELISMQESFDTSTAFGSATVGMLS |
| | VFAQLERKSISERMITGRVERAKKGFYHTGGQDRPPAGYQFNSDNQLIINEYEAAAIKDLFRLYND |
| | GLGKSSISEYLKKNYPGKNKWLPSSIDRMLKNSLYIGKVKFSGAEYDGIHEPIIDEVTFYKTQKEIAR |
| | RKQSNTKRYNYVALLGGLCECGICGAKMANRRSVGRKGKVYRYYRCYSKKGSPKHMMKTDGCS |
| | SKAQQQFIIDEAVINNLKNIDVEAELKRRSAPQTNTSLISSQIESIDKQINKLIDLFQVDSMPLDVISEK |
| | IDKLNKEKQSMEKLLERKNRLDKTELQHRFDILKSFDWDNSSIESKRAVIEMLVQKVIIHDNSIEIILV |
| | E |
| 91 | MKGESELDKKAAIYIRVSTQEQATEGYSIQAQTDRLIKYVEAKDFILYKKYIDAGYSASKLERPAM |
| | QDLIQDVQSKKVDVVIVYKLDRLSRSQKDTMYLIEDIFRPNDVELISMQESFDTSTAFGSATVGMLS |
| | VFAQLERKSISERMITGRVERAKKGFYHTGGQDRPPAGYQFNSDNQLIINEYEAAAIKDLFRLYND |
| | GLGKSSISEYLKKNYPGKNKWLPSSIDRMLKNSLYIGKVKFSGAEYDGIHEPIIDEVTFYKTQKEIAR |
| | RKQTNTKRYNYVALLGGLCECGICGAKMANRRAVGRKGKVYRYYRCYSKKGSPKHMMKTDGCS |
| | SKAQQQFIIDEAVINNLKNIDVEAELKRRSAPQTNTSLISSQIESIDKQINKLIDLFQVDSMPLDVISEK |
| | IDKLNKEKQSMEKLLERKNKLDKTELQHRFDVLKSFDWDNSSIESKRVVIEMLVQKVIIHDNSIEIIL |
| | VE |
| 92 | MRTGLYVRVSTAEQEKHGYSIKVQLEKLRAFASAKDYTVVKEYIDAAQSGAKLERPGLKQLIEDV |
| | ENNALDCVLVYRLDRLSRSQKDTMYLIEDVFLKNSVAFVSLQESFDTTSSFGRAMIGMLSVFAQLE |
| | RDNITERLFSGRAHRAKRGFHHGGGIIPFGYRYDVETGELKRFENESNEVKAMFEMIANGKSVSSV |
| | AKEFNTYDTTIRRRIANSVYIGKIQFDGETFDGQHEPIISKELFDKANVRMNARASNLPFKRTYLLSG |
| | LIYCGKCGERCSAYESRSKHNGKEYRRAYYRCNARTWKYKQKHGRTCEQPHIRVDELEQAVMEQ |
| | VKRLPLKHKVKKRAFDFKPVENKIATIDKQKERLLDLYLNEHLDNEMFNKKSKELDKSRDKLAKQ |
| | LERMRMQAADSVESYQWLDGIDWDALDKDTLREVLERIIERIVIRDKDVEIYFK |
| 93 | MKGESELDKKAAIYIRVSTQEQATEGYSIQAQTDRLTKYVEAKDFILYKKYIDAGYSASKLERPAM |
| | QELIQDVKSKKVDVVIVYKLDRLSRSQKDTMYLIEDIFRPNDVELISMQESFDTSTAFGSATVGMLS |
| | VFAQLERKSISERMITGRVERAKKGFYHTGGQDRPPAGYQFNSDNQLIINEYEAAAIKDLFRLYND |
| | GLGKSSISEYLKKNYPGKNKWLPSSIDRMLKNSLYIGKVKFSGAEYDGIHEPIIDEVTFYKTQKEIAR |
| | RKQTNTKRYNYVALLGGLCECGICGAKMANRRAVGRKGKVYRYYRCYSKKGSPKHMMKTDGCS |
| | SKAQQQFIIDEAVINNLKNIDVEAELKRRSAPQTNTSLISSQIESIDKQINKLIDLFQVDSMPLDVISEK |
| | IDKLNKEKQSMEKLLERKNKLDKTELQHRFDVLKSFDWDNSSIESKRVVIEMLVQKVIIHDNSIEIIL |
| | VE |
| 94 | MTVGIYIRVSTEEQVKEGFSISAQKEKLKAYCTAQGWEDFKFYVDEGKSAKDMHRPLLQEMISHIK |
| | KGLIDTVLVYKLDRLTRSVVDLHNLLSIFDEFNCAFKSATEVYDTSSAMGRFFITIISSVAQ1-ERENT |
| | SERVSFGMAEKVRQGEYIPLAPFGYTKGTDGKLIVNKIEKEIFLQVVEMVSTGYSLRQTCEYLTNIG |
| | LKTRRSNDVWKVSTLIWMLKNPAVYGAIKWNNEIYENTHEPLIDKATFNKVAKILSIRSKSTTSRRG |
| | HVHHIFKNRLICPACGKRLSGLRTKYINKNKETFYNNNYRCATCKEHRRPAVQISEQKIEKAFIDYIS |
| | NYTLNKANISSKKLDNNLRKQEMIQKEIISLQRKREKFQKAWAADLMNDDEFSKLMIDTKMEIDA |
| | AEDRKKEYDVSLFVSPEDIAKRNNILRELKINWTSLSPTEKTDFISMFIEGIEYVKDDENKAVITKISF |
| | L |
| 95 | MKAAIYIRVSTQEQVENYSIQAQTEKLTALCRSKDWDVYDTFIDGGYSGSNMNRPALNEMLSKLH |
| | EIDAVVVYRLDRLSRSQKDTITLIEEYFLKNNVEFVSLSETLDTSSPFGRAMIGILSVFAQLERETIRD |
| | RMVMGKIKRIEAGLPLTTAKGRTFGYDVIDTKLYINEEEAKQLRLIYDIFEEEQSITFLQKRLKKLGF |
| | KVRTYNRYNNWLTNDLYCGYVSYKDKVHVKGIHEPIISEEQFYRVQEIFSRMGKNPNMNKESASL |
| | LNNLVVCSKCGLGFVHRRKDTVSRGKKYHYRYYSCKTYKHTHELEKCGNKIWRADKLEELIIDRV |
| | NNYSFASRNIDKEDELDSLNEKLKIEHAKKKRLFDLYINGSYEVSELDSMMNDIDAQINYYEAQIEA |
| | NEELKKNKKIQENLADLATVDFNSLEFREKQLYLKSLINKIYIDGEQVTIEWL |
| 96 | MRKVAIYSRVSTINQAEEGYSIQGQIEALTKYCEAMEWKIYKNYSDAGFSGGKLERPAITELIEDGK |
| | NNKFDTILVYKLDRLSRNVKDTLYLVKDVFTANNIHFVSLKENIDTSSAMGNLFLTLLSAIAEFERE |
| | QIKERMQFGVMNRAKSGKTTAWKTPPYGYRYNKDEKTLSVNELEAANVRQMFDMIISGCSIMSIT |
| | NYARDNFVGNTWTHVKVKRILENETYKGLVKYREQTFSGDHQAIIDEKTYNKAQIALAHRTDTKT |
| | NTRPFQGKYMLSHIAKCGYCGAPLKVCTGRAKNDGTRRQTYVCVNKTESLARRSVNNYNNQKIC |
| | NTGRYEKKHIEKYVIDVLYKLQHDKEYLKKIKKDDNIIDITPLKKEIEIIDKKINRLNDLYINDLIDLP |
| | KLKKDIEELNHLKDDYNKAIKLNYLDKKNEDSLGMLMDNIDIRKSSYDVQSRIVKQLIDRVEVTMD |
| | NIDIIFKF |
| 97 | MKVAVYCRVSTLEQKEHGHSIEEQERKLKSFCDINDWTVYDTYIDAGYSGAKRDRPELQRLMNDI |
| | NKFDLVLVYKLDRLTRNVRDLLDLLEIFEKNDVSFRSATEVYDTTTAMGRLFVTLVGAMAEWERE |
| | TIRERTQMGKLAALRKGIMLTTPPFYYDRVDNKFVPNKYKDVILWAYDEAMKGQSAKAIARKLN |
| | NSDIPPPNNTQWQGRTITHALRNPFTRGHFDWGGVHIENNHEPIITDEMYEKVKDRLNERVNTKKV |
| | RHTSIFRGKLVCPVCNARLTLNSHKKKSNSGYIFVKQYYCNNCKVTPNLKPVYIKEKEVIKVFYNY |
| | LKRFDLEKYEVTQKQNEPEITIDINKVMEQRKRYHKLYASGLMQEDELFDLIKETDQTIAEYEKQN |
| | ENREVKQYDIEDIKQYKDLLLEMWDISSDEDKEDFIKMAIKNIYFEYIIGTGNTSRKRNSLKITSIEFY |
| 98 | MKVAIYTRVSTLEQREKGHSIDEQERKLRSFCDINDWTVKDVYVDAGFSGAKRDRPELTRLLDDIS |
| | EFDLVLVYKLDRLTRSVRDLLDLLEVFENNNVAFRSATEVYDTTTAIGRLFVTLVGAMAEWERETI |
| | RERSLMGKRAAIKKGMILTAPPFYYDRVNNTYIPNQYKDVVLDVYNKVKKGYSIAHIARLYNNSD |
| | VKPPNGNEEWTTRMLMHALRNPVTRGHYQWGEIYIEDSHEPIITDEMYNTIIDRLDKHTNTKVVAH |
| | TSVFRGKLICPNCGYALTLNSQKRKRKNDTIVYKTYYCNNCKITKGMKPHHITETETLRVFKDHLS |
| | KIDLKQYETQEKEKQSHVTIDLSKVMEQRKRYHKLYASGMMQENELFELIKETDEMIEEYEKQRK |
| | QVDVKEFDICKIKEIKDVLLKSWDIFTLEDKADFIQMSIKAINIEYTKLKRGKSSNSMKIKDIEFY |
| 99 | MPKVSVIPAKQVQVINGIKDKKKKRVCAYCRVSTDTDEQLTSYEAQVTYYESYIRGKPEYEFAGIF |
| | ADEGITGTNTKHRTEFKRMIDEALAGKFDMIITKSISRFARNTLDCLKYVRLLRDKGIGVYFEKENID |
| | TLDSKGEVLLTILSSLAQDESRNISENSRWGIVRRFQQGKVRVNHKRFLGYDKDENGELIIDEEQAKI |
| | VRRIYKEYLEGKGIRAIGKDLERDNILTGAGGRKWHDSTIQKILRNEKYSGDALLQKTITTDFLTHK |
| | RVKNKGEVQQYYVEDSHPAIISKEMFRMVQEEIKRRASLIGYSEKTKSRYTNKYAFSGRIVCGNCG |
| | SKFRRKRWGPGEKYKKYVWLCANHIDNGLKACSMKAVSEEKLKAAFVRSINKIIENKEAFIKTMM |
| | ENISRVSESKEDRSELKIINESLEELKEQMMNLVRLNVRSSLDNQIYDEEYERLEEEIKQLKEKKAGF |
| | DNTELIKKEGIQEVKEIERILRDRQDIIKDFDRELFMQIVDKVKVISLVEVEFIYKSGVVVKEIL |
| 100 | MKVAIYVRVSTDEQAKEGFSIPAQRERLRAFCASQGWEIVQEYIEEGWSAKDLDRPQMQRLLKDIK |
| | KGNIDIVLVYRLDRLTRSVLDLYLLLQTFEKYNVAFRSATEVYDTSTAMGRLFITLVAALAQWERE |
| | NLAERVKFGIEQMIDEGKKPGGHSPYGYKFDKDFNCTIIEEEADVVRMIYRMYCDGYGYRSIADRL |
| | NELMVKPRIAKEWNHNSVRDILTNDIYIGTYRWGDKVVPNNHPPIISETLFKKAQKEKEKRGVDRK |
| | RVGKFLFTGLLQCGNCGGHKMQGHFDKREQKTYYRCTKCHRITNEKNILEPLLDEIQLLITSKEYF |
| | MSKFSDRYDQQEVVDVSALTKELEKIKRQKEKWYDLYMDDRNPIPKEELFAKINELNKKEEEIYSK |
| | LSEVEEDKEPVEEKYNRLSKMIDFKQQFEQANDFTKKELLFSIFEKIVIYREKGKLKKITLDYTLK |
| 101 | MELSRNITVIPARKRVGNTAAAEQRPKLKVAAYCRVSTDSEEQASSYEVQVAHYTQFIQKNPEWEL |
| | AGIYADDGITGTNTKKREEFNRMIQDCMDGNIDMIITKSISRFARNTLDCLKYIRELKEKNIPVFFEK |
| | ENINTMDSKGEVLLTIMASLAQQESQSLSQNIKLGLQYRFQNGEVRVNHSRFLGYTKDEEGNLIIEP |
| | AEAEVVKRIYREYLEGASLLQIGRGLEADGILTGAGKTKWRPETLKKILQNEKYIGDALLQKTYTID |
| | FLSKKRVKNNGIVPQYYVENSHEPIIPRELFMQVQEEMVRRANLRGGKGGKKRVYSSKYALSSIVY |
| | CGQCGDIYRRVHWNNRGYKSIVWRCVSRLEEKGSECTAPTINEETLQAAVVKAINELLTKKEPFLS |
| | TLQKNIATVLNEENDNTTDDIDRKLEELQQQLLIQAKSKNDYEDVADEIYRLRELKQNALVENAER |
| | EGKRQRIAEMTDFLNEQSCELEEYDEQLVRRLIEKVTVFDEKMTIEFKSGVTIEGRI |
| 102 | MSVKKIRVNKQKNKQRICAYIRVSTTNGSQLESLENQKQYFINLYSNRDDIDFVGVYHDRGISGSK |
| | DNRPNFQAMIENCRKGMIDVIHTKSIARFARNTVTVLEISRELKAIGVDIFFEEQNIHTLSSEGEVML |
| | SVLASIAEDELRSMSGNQRWAFQKKFQRGELVINTKRFLGYDLDENGELIINPEEALIVRQIFALYLE |
| | GYGTHRIAKLLNEKGVATVTGAKWHDTTIRQMLSNEKYNGSVLLQKYFHDGVNGPKKLNQGELE |
| | QYFIEDNHEAIISMEDWQTVQAKLNRRRWQQGRNKTYKFTGLLKCQHCGSTLKRQVSYKKKIVW |
| | CCSKYIKEGKAACQGMRVPEVDISNWTVTSPVKVIERDRDGEKYYSYSSQESADQYSSSGQEENQS |
| | SRILSSVHRPRRTAIKL |
| 103 | MKPRQWAAENTEEKPKLKVAAYCRVSTEMEEQASSYEAQVQHYTDYIQRNPDWELAGIFADEGIS |
| | GTGTKKRDGFNRMIEACQKGDVEYIITKSISRFARNTVDCLQYIRQLKDLHIAVFFEKENINTMDAK |
| | GEVLLTIMASLAQQESQSLSQNTKMGVQYRFQQGQLRINHNHFLGYTKDEDGNLVIEPKEAEVIKR |
| | IFREYLEGSSLQEIANGLMSDGILTGGKRKLWRGEGVRLILRNEKYMGDALLQKTYTTDFLTKKRV |
| | KNDGSYAQQYYVENSHPAIIPRDIFMQVQQELDRRKSMKNKHSQCFSGKYALSGITVCGDCGNAY |
| | RRVHWKNRGTVWRCKSRVDKREHNCSGRTIYEKDLHEAIIKAINETVVDREDFLQQLSENINSVLT |
| | DGLTGRLEELDSKLKELESEIISMAIGGQGYDELASQIFSLRDERDAVAKQIAANTNLQQRVDEMVV |
| | FVKEHDVINEYSEVLVRRLIEKVTIFEKNIVVDFKSGVRVTVEI |
| 104 | MKAAIYIRVSTQEQIENYSIQAQTEKLTALCRSKDWDVYDIFIDGGYSGSNMNRPALNEMLSKLHEI |
| | DAVVVYRLDRLSRSQRDTITLIEEYFLKNNVEFVSLSETLDTSSPFGRAMIGILSVFAQLERETIRDR |
| | MVMGKIKRIEAGLPLTTAKGRTFGYDVIDTKLYINEEEAKQLQMIYDIFEEEKSITTLQKRLKKLGF |
| | KVKSYSSYNNWLTNDLYCGYVSYADKVHTKGVHEPIISEEQFYRVQEIFSRMGKNPNMNRDSASL |
| | LNNLVVCGKCGLGFVHRRKDTISRGKKYHYRYYSCKTYKHTHELEKCGNKIWRADKLEELIIDRV |
| | NNYSFASRNVDKEDELDNLNEKLKTEHKKKKRLFDLYISGSYEVSELDAMMADIDAQINYYEAQIE |
| | ANEELKKNKKIQENLADLATVDFDSLEFREKQLYLKSLINKIYIDGEQVTIEWL |
| 105 | MPTRIILPKPEESKKKRTAAYCRVSSSSEEQLHSLAAQTSYYENFFASAKDAEFAGIYADSGLSGTRT |
| | KNRTEFLRLIEDCRAGMVDAIITKSVSRFGRNTVDTLVFTRELRNLGIDVEFEKEDLHSCSPEGELLL |
| | TLMAAMAESEVVSMSDNIKWGKRKRFEKGMIESLALNNIYGFRKTADGIDIFETEACVVRHIYELF |
| | LSGLGYAEIAKRLNAENAPTRRDGSVWESTTVKNIITNEKNCGNCLFQKTFIRDPLSHKSRPNKGEL |
| | PQFLVEDCLPSIIDKETWLIAQRMRERNHRNGSSVPSEEYPFAGMLFCGICGAPVGFYYSKGEGFVM |
| | KTVYRCSSRKTRTAKAVEGVTYTPPHKSNYTKNPSPGLIEYREKYSGQYLQPRPMICTDIRIPLDRP |
| | QKAFVQAWNYIVGQRGRYHATLKRTVENNDDVLVRYRAREMLELFDGVGRLNTFDFPLMLRTLD |
| | RVETTKDEKLTFIFQSGIRITI |
| 106 | MSNKNVTVIPAKPTGFMQGLPGLITKRKVAGYARVSTDKDEQQNSYEAQVEYYTDYIKRNPEWEF |
| | VEVYTDEGISGTSTKHREGFKRMIADALDGKIDLILTKSVSRFARNTVDSLTTIRQLKDKGTEVYIAL |
| | KENIFTMDSKGELLLTIMSSLAQEESRSISKNITWGKRKSMADGKVSFAYSSFLGYDMGADGHLYI |
| | VEDQAKIVHRIYDEFLAGKTTYDIAVRLTEDGIPTPMNKVKWQASTVSNILQNVKYRGDSILQQYF |
| | VEDFLTKKIKKNTGELPLYYVSQNHPPIIPPEKFLMVQEEFRRRKEGGPYTCISPFSGRIVCGNCGGF |
| | YGRKVWHSGSSYQSFVWHCNNKFTKRKYCSTPSVKEDAIMKCFVDAFNNLIARKDEIARNYEECL |
| | AAITDDSAYKTRLAEVENLSAGLATRMHDNLTRESRMMDDCGEDSPIKKERDEITVEYEALQKEH |
| | KELNSKIALCAAKKVQVRGFLQLLKKQKKALVEFDPLVWQAAVHYMVINEDCTVKFVFRDGTEL |
| | PWVIDPGVKSYKKRKTVESCPQE |
| 107 | MEKQIIDITPTRTAFAVKQRVAAYARVSCDKDTMLHSLAAQIDYYRKYITRNPEWMFVGVYADEA |
| | KTGTKDDREQFQKLLSDCRSGLIDMVVTKSISRFARNTVTLLGTVRELKEIGINVFFEEQNINSISEE |
| | GELMLTLLASQAQEESLSCSENCKWKIRKGFERGQPNTCTMLGYRLVNGEITLVPDEAEIVKEIFDL |
| | YLSGCGVQKIANTLNKRSVRTEKIPFWHLDTIRGILRNEKYMGDLLLQKSLSESHLTKRQVKNEGQ |
| | LQQFYINDDHEPIVSRTVFAETQSEVQRRAEKHKCKAGTKSVFTGKIRCGICGKNYRRKTTPHNIV |
| | WCCSTFNTRGKAFCASKAIPENTLKDCISHALGSKYFTEDFFTETVDFIVAEPCNTMRLIFKNGTEK |
| | RITWQDRSRSESWTDEMREAVRQRMLERDGQKNEQ |
| 108 | MTPAQAPATFQGSHVDTDGEPWLGYIRVSTWKEEKISPELQETALRAWAARTGRRLLEPLIIDLDA |
| | TGRNFKRRIMGGIQRVEAGEARGIAVWKFSRFGRNNLGIAVNLARLEHAGGQLASATEDIDVRTAV |
| | GRFNRRILFDLAVFESDRAGEQWKETHQWRRAHGVPATGGRRLGYTWHPRRIPHPTLIGQWATQR |
| | EWYEVEESARTHIERLYARKIGTDLRAPEGYGSLSAWLNSLGYRTGNGNPWRADSVRRYMLSGFA |
| | AGLLRIHDLECRCDYTANGGQCIRWTHIDGAHEAIITPETWERYVAHVAERRRMAPRVRNPTYPLT |
| | GLIRCGGCREGAAATSARRAAGQILGYAYACGQSRSGLCDSPVWVQRAIVEDELLLWISREVAAE |
| | VDAAPPTGIPQQRDDGTERTQAERARLEGEHTRLTNALTNLAVDRATNPEKYPDGIFEAAREQILQ |
| | QKRAVSEALEAHTMVAALPQRSTLIPLAVGLLDEWDTFHPPETNGILRSLLRRVVITRGAAGRKGV |
| | RGSAQTKIEFHPAWEPDPWEGLE |
| 109 | MKVAIYLRVSTQEQVDNYSIEAQRERLEAFCKAKGWTVYDVYVDAGFTGSNTDRPGLQRLLMEL |
| | DKVDVVAVYKLDRLSRSQRDTLTLIEDHFLKNKVDFVSLTEALDTSTPFGKAMIGILAVFAQLERET |
| | IAERMRLGHIKRAEEGLRGMGGDYDPAGYKRQDGRLVLVPEEAQHIQEAFNLYEQYLSITKVQKR |
| | LKELNYPVWRFRRYRDILSNKLYCGYVQFADKHYKGQHESIITEEQFDRVQILLSRHKGRNAFKAK |
| | EALLTGLAVCGECGESYVSYHCRAKGKHYRYYTCRARRFPSEYPEKCHNKNWRSEAIEKFIQDAL |
| | YTIADEKETSEREFVAIDYGTQLKKIDQKLERLVDLYADGSIEKSVLDKQVTKLNNEKRDIAEQQA |
| | AQTERAARSVNRKQLQDYAIVLESAAFPDRQAIVQKLIRRLAIHKDRLEIEWNF |
| 110 | MRICMYLRKSRADEELEKTLGEGETLSKHRKALLKFAKEKNLNIVEIKEEIVSGESLFFRPKMLELL |
| | KEIENKQYSGVLVMDMQRLGRGNMQDQGIILETFKKSNTKIITPMKTYDLSNDFDEEYSEFEAFMS |
| | RKELKMINRRMQGGRVRSVEDGNYIATNAPYGYDIHWINKARTLKPNQKESEIVKLIFKLYIEGNG |
| | AGTIAKHLNSLGYKTKFENSFNNSSIIFILKNPVYIGKITWKKKDIRKSKDPNKIKDTRTRDKSEWIV |
| | VDGKHDPIIDQITWKQAQEILNNRYHIPYKLVNGPANPLAGLIICATCKSKMVMRKLRGTDRILCKN |
| | NKCNNISNRFDAVEKSVVESLENYLKAYKVNLPELNEISNLKLYEQQISTLKKELKILNEQRLKLFD |
| | FLERGIYDEDTFLKRSKNLDERIEITNESLSNLNQIIAKENKAIKKEDIIKFEKVLDSYKSTADIRLKNE |
| | LMKTLIFKIEYTKNKKGNDFKIKVFPKLKPLNI |
| 111 | MKCVIYRRVSTDMQVEEGISLDMQKLRLEQYAKSQGWVVVNDYCDEGYSAKNTERPAFQKMIKD |
| | MKKKQFDIILVYRLDRFTRSVSDLHSILKIMDEYNVKFKSSTEIFDTTTATGRMFITLVATLAQWER |
| | ETTAERVRDSMHKKAELGLRNGAKSPMGYDLNKGNLYINHTEAEIVKYIFEMFKTKGIISIVKSLNS |
| | RGVKTKRGKIFNYDAVRYIINNPIYIGKIRWGDDILTDIAQKDFETFIDKDTWYTVQQVQDSRKRGK |
| | VRLHNFFVFSNVLKCARCGKHFLGNKQVRSHNRIVMSYRCSSRHHKGTCDMPQVPEDVIEKEFLN |
| | LLEDAIVDLDDTEEKPIELSNLQEQYNRIQDKKARLKYLFIEGDIPKNEYKKDMLTLTQEENIIQKQL |
| | ANITDTASSLEIKELLNQLKDEWYNLNNESKKAAVNAIVSSITVEVTKPARVGKNPIAPVIKVTDFKI |
| | K |
| 112 | MKTAIYLRKSRADLEAEARGEGETLAKHRSTLLKIAKEMNLNVLAVREEIVSGESLVKRPEMLALL |
| | EEIEDNKYDAVLCMDMDRLGRGGMKEQGIILETFKRSNTKIMTPRKTYDLNDEWDEEYSEFEAFM |
| | ARKELKIITRRMQRGRVASVEAGNYLGTHAPFGYDIHRLNKRERTLTINSEEASVVRMIFDWYANE |
| | EMGANAIRSKLNDLGYKSKLGNEWNPYSILDILKNNVYIGKVTWQKRKEVKRPDAVKRSCARQD |
| | KSDWIIADGKHEPIISESLFEQVQDKLNSRYHVPYNTNGIKNPLAGIIKCGKCGYSMVQRYPKNRKE |
| | AMDCKHRGCENKSSYTELIEKRLLEALKEWYVNYKADFEKHKQDDKLKETQVIQMNEVALRKLE |
| | KELVDVQKQKNNLHDLLERGVYTVDMFLERSNVISDRINEITSTMEKLQNEIKTEIKKEKVKKDTIP |
| | QVEHVLDLYFKTDDPKKKNSLLKSVLEKAVYKKEKWQRLDDFELVLYPKLPQDGDI |
| 113 | MASHSSWEIHPDLAAALASGKTVEEWLDGRTPVVSYARISVDLQKVKAIGVARQHGMHCDPAAK |
| | EQGWAVVYRYTDNDLTAADPDVQRPAFLQMVRDLRARQTAEGIAIRGILAVEEERVVRLPEDYLK |
| | LYRALTVEEDAVLYYTDKRQLVDVYAEVEQTRGLMSSSMGETEVRKVKRRAKRSTKDRAAEGKY |
| | TGGARRFGWLGADKDLGRTQNEKLDPDESVWLRNMIDMKLCGKGWHTIAVWLISESIATVRGGE |
| | WTSTGVKSLLTNPAICGYRILNGELVLDPGTGEPKVGNWETIATPEEWHQICEMAWPGGKLAKTK |
| | KPKGTKRARKHLSTGILRCGWIPKSGPKEDMCLHSMVGRPPHGNHKWGNYVCNGTDCRKVSRRM |
| | DKIDRIVEGIVVRTLKDQFATLAPEEKTWHGQHTLERLTARRQELKAAYKAEHISMADYLEFIDPL |
| | DAQIKESQADRDAFYAEQAAKNFLAGFTEERWHDFDLEQKQTAIGTVLQAVIVHPLPEGRSRKAPF |
| | DPSLIEIVFKNPH |
| 114 | MAKELTKTASVAAYLRKSREDADQDDTLARHRKQLIDLVKQRGFENVDWYEEIGSADSIKNRPVF |
| | SDLLKKIENDEYDAVCVVAYDRLSRGNQIESGIISKAFKDTETLLITPTRTYDWSIEGDEMLSEFESM |
| | IARSEYRVIKKRLKQGKINAVKNGRLHSGNVPYGYKWDKNDKTAKIDKEKHEIYRLMVKWFLDEE |
| | YSATEIADKLNELGIPSPSGGSTWYSEVVADILTNDFHRGLVWYGKYRARKNGIGIEKNPDSSSIIM |
| | HKGNHEPMKSDEEHGAIIRRISKLRTFKPGRKLNKNTFKLSGLVRCPHCGKVQVVHTPKNRNPHVR |
| | KCLKKSKTRTTECNNTTGIPEEALYKAIVMKIREYNEVLFSKDSSEKKDEEARTYMNQILSLHEKAI |
| | SKSNKRIEKIKEMYMDEIIDKDEFKSRIDKEKKSILEAENEIRTLKESADYHDEIEHEQRKIKWNHEK |
| | VQEFIESDQGFTPSEINLILKLIISHVSYTMVKNEYGEFDVDLRVNFN |
| 115 | MGKNGARVYSYLRFSDPRQATGSSADRQLAYASAWASKHGMELDATLTLRDEGLSAYHETHVK |
| | QGALGAFLRAVDEGRIPAGSVLIVEGLDRLSRAEPLLAQAQLGQIVNAGITVVTASDGREYNREGL |
| | KAEPMNLVYSLLVMIRAHEESDTKSKRVKAAVRRQCEAWVTGSYRGRIVSGKDPQWLAWDGDS |
| | WQFIPERVEAVRFALDAYRSGIGAARLVRLMHEKGMVLSDWGIAAQQVYRLVRLPALRGAKRISI |
| | DGEDFMLEDYYPRLLSDEEFSELETLVGQRYRRRGKDEIVGIVTGIGITRCGYCGTALVAQNLMQR |
| | VKADGSLEDGHRRLHCVSYSKNGGCNGGSCSSVPIERAVLAYCSDQMNLQRLLEPSSAGEDLRPR |
| | LVEAQKGVAEIERQLERVTDALLADDSGAAPLAFVRKARELEEDLERRRSAVQALEQELVAKSAS |
| | APAAGASKWAELAERAKSMADAEAREQARQLVMDTFETLVVYMRGVIPNPKGRYIDVMMKSRA |
| | GQTRWIRVDRRTGVWKEGADRPTTRRP |
| 116 | MSIAIYLRKSRADEEAEKQGEFETLSRHKSTLLKLAKEQNLDVIEIKEELVSGESIIHRPKMLELLKE |
| | VEENKYDAVLVMDLDRLGRGDMKDQGIILETFKESKTKIITPRKTYDLTDEFDEEYSEFEAFMARK |
| | ELKLISRRMQRGRVKSVEEGNFIGTSAPFGYDAVTTGRKERILVPNKDADIVRTIFDLYINEDMGCS |
| | KISKYLNNLGIKTATGANWYNSAITNIIKNKVYCGYIQWQKKDYKKSKNPNKIKTVKLRPKDEWIE |
| | AKGKHEPLISEITWKKAQNILKKNGHVSYGNQIKNPLAGIVICKNCGRPLVYRPYADHDYIICYHPG |
| | CNKSSRFEFIEAAILKSLEDTVKKYQLKASDIDLDKNNKGSNIEFQKRVLKGLETELKELSKQKNKL |
| | YDLLERGIYDEDTFIERSNNISSRTEEIKDSIKTVKNKLNSVKKDNAKIIEDIKTVLSLYHDSDSLGKN |
| | KLLKSVIDKAIYYKSKEQKLDSFELMVHLKLHEDQ |
| 117 | MKVPVWCYARISTLKQIDGFGIQRQINTINQFLQYVVLDHRLPFTLDVDNVTQMVAEGKSAFRGK |
| | NWNEKTKLGQYRKMVMDGVINDSVLIVENIDRLTRLDTFQAVEIISGLVNRGTTILEIETGMTYSRY |
| | IPESITVLVMQCNRANGESKRKSIMMQKSHANRYGKVSKVRPRWFDVVEIDGIKQYRPNETAKAIQ |
| | RMYNDYINGIGAAHIVRTYGNTDNGKAWTLVTVLRALSDKRVADDARYPPIIDKELYDSVQALKA |
| | ATNKKGNTHQKNMLNIFSGMSRCPVCNQSIIVKRNSHGNLFTVCLGKRTNKTCEARSISYFALERPL |
| | LTAISGLDFSEVYKHEDKNVLTLRDQWIQNERDIAAFRERLNKASRHEKFAILDELEIMNREQEELTI |
| | RLKSVDVPKDIQLTFDDDKLDLDTNYRIELNNRIKKLIQHINIVREDVSKSSYTIYCTIKYWTDVISHL |
| | VIIDVNIKRTGTGGTNTLTTTLRSVSSLNMDGTVSGNPDSDAWEYWKSFLDGTIGLVDYKK |
| 118 | MRKVAIYSRVTTINQAEEGYSIQGQIEALTKYCEAMEWKIYKNYSDAGFSGGKLERPAITELIEDGK |
| | NNKFDTILVYKLDRLSRNVKDTLYLVKDVFTANNIHFVSLKENIDTSSAMGNLFLTLLSAIAEFERE |
| | QIKERMQFGVMNRAKSGKTTAWKTPPYGYRYNKDEKTLSVNELEAANVRQMFDMIISGCSIMSIT |
| | NYARDNFVGNTWTHVKVKRILENETYKGLVKYREQTFSGDHQAIIDEKTYNKAQIALAHRTDTKT |
| | NTRPFQGKYMLSHIAKCGYCGAPLKVCTGRAKNDGTRRQTYVCVNKTESLARRSVNNYNNQKIC |
| | NTGRYEKKHIEKYVIDVLYKLQHDKEYLKKIKKDDNIIDITPLKKEIEIIDKKINRLNDLYINDLIDLP |
| | KLKKDIEELNHLKDDYNKAIKLNYLDKKNEDSLGMLMDNLDIRKSSYDVQSRIVKQLIDRVEVTM |
| | DNIDIIFKF |
| 119 | MGKNGARVYSYLRFSDPRQATGSSADRQLAYASAWASKHGMELDATLTLRDEGLSAYHETHVK |
| | QGALGAFLRAVDEGRIPAGSVLIVEGLDRLSRAEPLLAQAQLGQIVNAGITVVTASDGREYNREGL |
| | KAEPMNLVYSLLVMIRAHEESDTKSKRVKAAVRRQCEAWVAGSYRGRIVSGKDPQWLAWDGDS |
| | WQFIPERVEAVRFALDAYRSGIGAARLVRLMHEKGMVLSDWGIAAQQVYRLVRLPALRGAKRISI |
| | DGEDFMLEDYYPRLLSDEEFSELETLVGQRYRRRGKDEIVGIVTGIGITRCGYCGTALVAQNLMQR |
| | VKADGSLEDGHRRLHCVSYSKNGGCNGGSCSSVPIERAVLAYCSDQMNLQRLLEPSSAGEDLRPR |
| | LVEAQKGVAEIERQLERVTDALLADDSGAAPLAFVRKARELEEDLERRRSAVRALEQELVAKSAS |
| | APAAGASKWAELAERAKSMADVAAREQARQLVMDTFETLVVYMRGVIPNPKGRYIDVMMKSRA |
| | GQTRWIRVDRRTGVWKEGADRPTTRRP |
| 120 | MQSPKVYSYFRFSDPRQAAGSSADRQLAYASAWASKHGMELDATLTLRDEGLSAYHETHVKQGA |
| | LGAFLRAVDEGRIPAGSVLIVEGLDRLSRAEPLLAQAQLGQIVNAGITVVTASDGREYNREGLKAEP |
| | MNLVYSLLVMIRAHEESDTKSKRVKAAVRRQCEAWVAGSYRGRIVSGKDPQWLTWGGDSWQFIP |
| | ERVEAVRFALDAYRSGIGAARLVRLMHEKGMVLSDWGIAAQQVYRLVRLPALRGAKRISIDGEDF |
| | MLEDYYPRLLSDEEFSELETLVGQRYRRRGKDEIVGIVTGIGITRCGYCGTALVAQNLMQRVKADG |
| | SLADGHRRLHCVSYSKNGGCNAGSCSSVPIEHAVLAYCSDQMNLQRLLEPSSADEELRPRLAEAQQ |
| | RVAEVERQLQRVTDALVADDSGAAPLSFVRKARELEEELERRRSAVRVLERELVAMASSVPVAEA |
| | SKWAELAEQAKSVSNVEAREQARQLVMDTFERIVVYMRGVVPEGRRSKYIDVLLVSRAGQSRWL |
| | RVGRRTGTWSAGGDWNGSAP |
| 121 | MKAAIYIRVSTQEQIENYSIQAQTEKLTALCLSKDWDVYDIFIDGGYSGSNMNRPALNEMLSKLHEI |
| | DAVVVYRLDRLSRSQRDTITLIEEYFLKNNVEFVSLSETLDTSSPFGRAMIGILSVFAQLERETIRDR |
| | MVMGKIKRIEAGLPLTTAKGRTFGYDVIDTKLYINEEEAKQLRLIYDIFEEEQSITFLQKRLKKLGFK |
| | VRTYNRYNNWLTNDLYCGYVSYKDKVHVKGIHEPIISEEQFYRVQEIFSRMGKNPNMNKESASLL |
| | NNLVVCSKCRLGFVHRRKDTVSRGKKYHYRYYSCKTYKHTHELEKCGNKIWRADKLEELIIDRVN |
| | NYSFASRNIDKEDELDSLNEKLKIEHAKKKRLFDLYINGSYEVSELDSMMNDIDAQINYYEARIEAN |
| | EELKKNKKIQENLADLATVDFNSLEFREKQLYLKSLINKIYIDGEQVTIEWL |
| 122 | MSVKKIRVNRQKHRKRVCAYIRVSTTNGSQLDSLENQKQYFENLYSNRDDIDFMGVYQDRGISGS |
| | KDKRPDFQAMIEECRKGKIDVIHTKSIARFARNTVTVLEISRELKAIGVDIFFEEQNIHTLSSEGEVML |
| | SVLASIAEDELRSMSGNQRWAFQKKFQRGELVINTKRFLGYDVDENGELIINPEEALIVRQIFALYLE |
| | GYGTHRIAKLLNEKGVATVTGAKWHDTTIRQMLSNEKYKGSVLLQKYFHDGVNGPKKLNQGELE |
| | QYLIEDNHEAIISKEDWQAVQDKLNSRRWQQGRNKTYKFTGLLKCQHCGSTLKRQVSYKKKIVW |
| | CCSKYIKEGKVACRGMRVPEVDIPNWEITSPITVLERDRNGEKYYSYSGQESEDQRSSSGQEENQGS |
| | RILSSVHRPRRTAIKL |
| 123 | MKTKLYSYIRFSSMRQNDGSSYERQIRMAREIAVKYDLELVNDYQDLGVSAFKGANSKTGALSRF |
| | LDAIGRSVPVGSWLFIENLDRLSRADIVSAQELFLSIIRRGITIVTGMDNKIYSLDTVTANPMDLMFSI |
| | LLFIRGNEESQTKRNRTNSSALIKIKAHQENPQNPAVAIEEIGKNMWWTDTTSGYVLPHPVFFPIVQ |
| | EVVELRRNGRSTAEILDHLNATYTPPPAASHKRHSNWSRAMIERLFHTRALIGIKEISVDGVKYELK |
| | DYYPRVLDDAEFYHLKKSIGVRACNFGDKEEAKPIPLLSGVGLLKCEHCGSAMVKVKGTNRRPNQ |
| | YRYSCDAMRSSRIECVHTNWSFRGDQLEKAVLQLLADKIWIAEDKANPVPALKVQIDEISRKIDNLI |
| | TLSAMTGATKELADQITTLNSERETLYNQLKMAEEEMYSVDSQGWEKLAEFDLEDVYNEDRIKVR |
| | FKIKQALKRIGCSRIDKYKNLFVLEYIDGKTQRVVIENSRGPRKGRIFVDLKTINDRQILESNGLVLH |
| | PCLDMLTDKNWKPEEEIPGPLQEFGI |
| 124 | MSVKKIRVNRQKHRKRVCAYIRVSTTNGSQLDSLENQKQYFENLYSNRDDIDFIGVYHDRGISGSK |
| | DNRPNFQAMIEDCRRGKIDVIHTKSIARFARNTVTVLEISRELKAIGVDIFFEEQNIHTLSSEGEVMLS |
| | VLASIAEDELRSMSGNQRWAFQKKFQRGELVINTKRFLGYDVDENGELIINPEEALIVRQIFALYLE |
| | GYGTHRIAKLLNEKGVATVTGAKWHDTTIRQMLSNEKYNGSVLLQKYFHDGVNGPKKLNQGELE |
| | QYFIEDNHEPIISMEDWQTVQEKLNSRRWQQGRNKTYKFTGLLKCQHCGSTLKRQVSYKKKIVWC |
| | CSKYIKEGKAACQGMRVPEVDISNWTVTSPVKVIERDRDGEKYYSYSCQESAEQRSTSGQKENQCS |
| | RILPSVHRSRRTAIKL |
| 125 | MKGESKLDKKAAIYIRVSTQEQATEGYSIQAQTDRLTKYVEAKDFILYKKYIDAGYSASKLERPAM |
| | QELIQDVQSKKVDVVIVYKLDRLSRSQKDTMYLIEDIFRPNDVELISMQESFDTSTAFGSATVGMLS |
| | VFAQLERKSISERMITGRVERAKKGFYHTGGQDRPPAGYQFNSDNQLIINEYEAAAIKDLFRLYND |
| | GLGKSSISEYLKKNYPGKNKWLPSSIDRMLKNSLYIGKVKFSGAEYDGIHEPIIDEVTFYKTQKEIAR |
| | RKQTNTKRYNYVALLGGLCECGICGAKMANRRAVGRKGKVYRYYRCYSKKGSPKHMMKTDGCS |
| | SKAQQQFIIDEAVINNLKNIDVEAELKRRSAPQTNTSLISSQIESIDKQINKLIDLFQVDSMPLDVISEK |
| | IDKLNKEKQSMEKLLERKNKLDKTELQHRFDVLKSFDWDNSSIESKRVVIEMLVQKVIIHDNSIEIIL |
| | VE |
| 126 | MKRVALYIRVSTEEQVLHGDSIRTQTEALEQYSKDNNFIIVDKYIDEGYSATNLKRPNLKRMIEDVK |
| | NNKIDLVMITKIDRLSRGVKNYYKIMETLEKHKCDWKTILEDYDSSTAAGRLHINIMLSVAENEAA |
| | QTSERIKFVFQDKLKRGEVITGSVPFGYKIKDKHLVIKEDEASIVREAFDAYQDFSSLAKTIQHINTK |
| | FSTKYMFKWMPKMLKNKIYIGIYEKGDLVVENYCEPIISREQFNFVQTLLKKNIRFSENKFKMNYLF |
| | SGMIVCGSCGRKMGGVHSRGGANRHYLYYRCPLSFATKLCDNKPYLNEKKVEAFLLENVKKELQ |
| | KTILEHESNNKKRQKKNNNKNLRNKLEKQIEKLQDLYFDDLINKDTYKFKYKKLNDDLSELNKAE |
| | NEAESVEKDLKSMKIFLDTNIALDNYYDMNYSEKRTLWTSAIDRIEVQKNGELVIKFL |
| 127 | MRKVTRIDGNNALQAFKPKVRVAAYCRVSTDSDEQMASLEAQKDHYESYIKANPDWEFAGIYYD |
| | EGISGTKKENRTGLLRLLADCENKKIDFIITKSVSRFARNTTDCIEMVRKLTDLGVFIYFEKENINTQR |
| | MEGELVLTILSSLAENESLSIAENSKWSIRRRFQNGTYKISYPPYGYDYVDGKLFINKEQAEIIKRIFS |
| | EALVGKGTQKIADGLNLDKIPTKRGSHWTATTIRGILSNEKYTGDVLLQKTYTDENFKRHYNRGEK |
| | DQYMIKDHHEAIISHEEFEAVKEILKQRGKEKGVIKGSSKYQNRYPFSGKIKCAECGSSFKRRIHGSG |
| | NHKYIAWCCTKHIKDASACSMKFVREDGIHQAFVVMMNKLIFGHKFILRPLLQSLKKTNYSDNITK |
| | IQELETKIKENTERVQVIMGLMAKGYLEPALFNTQKNELSKEAALLKEQKEAINRAINGSQTILVEV |
| | EKLLKFATKAEKQIDAFDSKIFEDFIEEIIVFSQEEISFKMKCGLNLRERLVK |
| 128 | MDTKVAIYVRVSTHHQIDKDSLPLQKQDLINYANYVLNTNNYEIFEDAGYSAKNTDRPGFQNMMS |
| | RIRNNEFTHLLVWKIDRISRNLLDFCDMYNELKKINVTFVSKNEQFDTSSAMGEAMLKIILVFAELE |
| | RKLTGERVTAVMLDRATKGLWNGAPIPLGYIWDKIKKFPVIDDAEKNTIELIYNTYLKVKSTTAIRS |
| | LLNANNIKTKRNGTWTTKTISDIIRNPFYKGTYRYNYREPGRGKVKSENEWVVIEDNHKGIISKELW |
| | RKCNAIMDENAKRNNAAGFRANGKVHVFAGLLECGECHNNLYSKQDKPNLDGFIPSVYVCSGRY |
| | NHLGCNQKTISDNYVGTFIFNFISNILKTQNKIKKLDSKLLEKALLNGNVFKDIIGIENIEDLQNKSYA |
| | SNVLKNKKNANEDNSFGLEVNKKEKAKYERALERLEDLYLFDDNAMSEKDYIIRKKKIAEKLNEV |
| | NEKLKELNTFADEQEINLLSKISSFTLSKELLNAYNIHYKELILNIGRNQLKDFANTIIDKIIIKDKKILN |
| | IKFKNNLKISFVHRG |
| 129 | MKAAIYIRVSTQEQVENYSIQAQTEKLTALCRSKDWDVYDIFIDGGYSGSNMNRPALNEMLSKLHE |
| | IDAVVVYRLDRLSRSQRDTITLIEEYFLKNNVEFVSLSETLDTSSPFGRAMIGILSVFAQLERETIRDR |
| | MVMGKIKRIEAGLPLTTAKGRTFGYDVIDTKLYINEEEAKQLRLIYDIFEEEQSITFLQKRLKKLGFK |
| | VRTYNRYNNWLTNDLYCGYVSYKDKVHVKGIHEPIISEEQFYRVQEIFARMGKNPNMNRDSASLL |
| | NNLVVCSKCGLGFVHRRKDTMSRGKKYHYRYYSCKTYKHTHELEKCGNKIWRADKLEELIIDRV |
| | NNYSFASRNVDKEDELDSLNEKLKTEHTKKKRLFDLYISGSYEVSELDAMMSDIDAQINYYEAQIE |
| | ANEELKKNKKIQENLADLATVDFDSLEFREKQLYLKSLINKIYIDGEQVTIEWL |
| 130 | MKAAIYIRVSTQEQVENYSIQAQTEKLTALCRSKDWDVYDTFIDGGYSGSNMNRPALNEMLSKLH |
| | EIDAVVVYRLDRLSRSQKDTITLIEEYFLKNNVEFVSLSETLDTSSPFGRAMIGILSVFAQLERETIRD |
| | RMVMGKIKRIEAGLPLTTAKGRTFGYDVIDTKLYINEEEAKQLQMIYDIFEEEKSITTLQKRLKKLG |
| | FKVKSYSSYNNWLTNDLYCGYVSYADKVHTNGVHEPIISEEQFYRVQEIFSRMGKNPNMNRDSAS |
| | LLNNLVVCGKCGLGYVHRRKDTVSRGKKYHYRYYSCKTYKHTHELEKCGNKIWRADKLEELIIDR |
| | VNNYSFASRNVDKEDELDSLNEKLKTEHAKKKRLFDLYISGSYEVSELDGMMADIDAQINYYEAQI |
| | EANEELKKNKKIQENLADLATVDFDSLEFREKQLYLKSLINKIYIDGEQVTIEWL |
| 131 | MKAAIYIRVSTQEQIENYSIQAQTEKLTALCRSKDWDVYDIFIDGGYSGSNMNRPALNEMLSKLHEI |
| | DAVVVYRLDRLSRSQRDTITLIEEYFLKNNVEFVSLSETLDTSSPFGRAMIGILSVFAQLERETIRDR |
| | MVMGKIKRIEAGLPLTTAKGRTFGYDVIDTKLYINEEEAKQLQMIYDIFEEEKSITTLQKRLKKLGF |
| | KVKSYSSYNNWLTNDLYCGYVSYADKVHTNGVHEPIISEEQFYRVQEIFSRMGKNPNMNRDSASL |
| | LNNLVVCGKCGLGYVHRRKDTVSRGKKYHYRYYSCKTYKHTHELEKCGNKIWRADKLEELIIDR |
| | VNNYSFASRNVDKEDELDSLNEKLKTEHAKKKRLFDLYISGSYEVSELDGMMADIDAQINYYEAQI |
| | EANEELKKNKKIQENLADLATVDFDSLEFREKQLYLKSLINKIYIDGEQVTIEWL |
| 132 | MGKNGARVYSYLRFSDPRQATGSSADRQLAYASAWASKHGMELDATLTLRDEGLSAYHETHVK |
| | QGALGAFLRAVDEGRIPAGSVLIVEGLDRLSRAEPLLAQAQLGQIVNAGITVVTASDGREYNREGL |
| | KAEPMNLVYSLLVMIRAHEESDTKSKRVKAAVRRQCEGWVTGSYRGRIVSGKDPQWLAWDGDS |
| | WQFIPERVEAVRFALDAYRSGIGAARLVRLMHEKGMVLSDWGIAAQQVYRLVRLPALRGAKRISI |
| | DGEDFMLEDYYPRLLSDEEFSELETLVGQRYRRRGKDEIVGIVTGIGITRCGYCGTALVAQNLMQR |
| | VKSDGSLEDGHRRLHCVSYSKNGGCNGGSCSSVPIERAVLAYCSDQMNLQRLLEPSSAGEDLRPRL |
| | VEAKKGVAEIERQLERVTDALLADDTGAAPMAFVRKARELEEDLERRRSAVRALEQELVTKSAST |
| | PAAGASKWAELAERAKSMTDVEAREQARQLVMDTFETLVVYMRGVMPTPKGRYIDLMMRSRAG |
| | QTRWLRVDRRSGVWRESGDSSRRLEG |
| 133 | MKVAIYTRVSSAEQANEGYSIHEQKRKLISFCEVNDWNRYEVFSDPGVSGGSMKRPSLQKLFDRLE |
| | EFDLVLVYKLDRLTRNVRDLLEMLEVFEKNNIAFKSATEVFDTNSAIGKLFITMVGAMAEWERETI |
| | RERSLMGSHAAIRSGKYIRARPFCYDLIDDKLKPNQHAKYIRFMVDKLMIGKSASEVVRQLESKKK |
| | PPGITKWNRKMILNWIKNPVMRGHTKFGDLLIENTHEPIISEDEYLKLIDIIEKRTYKTKSKHKAIFRG |
| | VLECPRCQSKLHLSRSIKKYDNGKTREVRRYSCDKCHRDNTVKNISFNESEIERQFINTLLKKGTDN |
| | FKISVPKKKSYDIEDNKVKINEQRANYTRSWSLGYIKDEEYFMLMDETENLLKDIEEKAKSHTDEK |
| | LNEEQIRTVKNLLIKGFKIATLEDKEDLITSSVDVIKFEFIPKEFNKNKTLNTVKINEIQFKF |
| 134 | MKVAIYTRVSSAEQANEGYSIHEQKRKLISFCEVNDWNRYEVFSDPGVSGGSMKRPSLQKLFDRLE |
| | EFDLVLVYKLDRLTRNVRDLLEMLEVFEKNNIAFKSATELFDTTSAIGKLFITMVGAMAEWERETIR |
| | ERSLIGARAAVRSGKYIKVQPFCYDLVDQKLKPNQYAEYIRFIVDKLLSGKSANEVVRLLESKKKPP |
| | GITKWNRKTVLGWMRNP1LRGHTKHGDLLIKNTHEPIISEDEHSKMLDIIDKRTHKSKTKHNSIFRG |
| | VIECPQCQNKLYLFSSIQKRANGGSYEVRRYTCATCHKNKEVKDVSFNESEIEREFINTLLKKGTDN |
| | FMVNIPKPKDYDIENNKEKILEQRTNYTRAWSLGYIKDEEYFVLMDETDKLLKDIEEKESPRINIELN |
| | EQQIRTVKNLLIKGFKMATAENKEELITSTVDLIKIDFIPRRLNKESNINTVKINEIHFKY |
| 135 | MAKVTTIPATISRFTATPINEKKKRRTAAYARVSTDSEEQLTSYSAQVDYYTNYIKSRDDWEFVSV |
| | YTDEGITGTNTKHREGFKRMVADALAGKIDLIVTKSVSRFARNTVDSLTTVRQLKEKGVEIYIALKE |
| | NIWTLDSKGELLITIMSSLAQEESRSISENCTWGQRKRFADGKVTVPFKRFLGYDRGPDGNLVLNK |
| | DEAVIIRRIYSMFLQGMTPHGIAARLTADGIKSPGGKDKWNAGAVRSILTNEKYKGDALLQKSYTV |
| | DFLTKKKKVNEGEIPQYYVEGNHEAIIQPEVFELVQQELERRKSSRGRHSGVHLFSGKIRCGQCGE |
| | WYGSKVWHSNSKYRRVIWQCNHKYDGEEKCSTPHLTEDEIKAMFVSAANKLIGKKAAIISPLRNSL |
| | DVAFDTSALETEVAELQDEIMVVSDLIEKCIYENAHVALDQTEYQKRYDGLTTRFDTAKARLEEIE |
| | AALADKKSRRAAIDAFLDTLAQADPMEKFDPALWCGLIDYVTVYARDDVRFAFKDGQEIKA |
| 136 | MGKNGARVYSYLRFSDPRQATGSSADRQLAYASAWASKHGMELDATLTLRDEGLSAYHETHVK |
| | QGALGAFLRAVDEGRIPAGSVLIVEGLDRLSRAEPLLAQAQLGQIVNAGITVVTASDGREYNREGL |
| | KAEPMNLVYSLLVMIRAHEESDTKSKRVKAAVRRQCEGWVTGSYRGRIVSGKDPQWLAWDGDS |
| | WQFIPERVEAVRFALDAYRSGIGAARLVRLMHEKGMVLSDWGIAAQQVYRLVRLPALRGAKRISI |
| | DGEDFMLEDYYPRLLSDEEFSELETLVGQRYRRRGKDEIVGIVTGIGITRCGYCGTALVAQNLMQR |
| | VKSDGSLEDGHRRLHCVSYSKNGGCNGGSCSSVPIERAVLAYCSDQMNLQRLLEPTSAGEDLRPRL |
| | VEAQKGVAEIERQLERVTDALLADDSGAAPLAFVRKARELEEDLERRRSAVQALEQELVAKSASA |
| | PAAGASKWAELAERAKSMADVDAREQARQLVMDTFETLVVYMRGVIPNPKGRYIDVMMKSRAG |
| | QTRWIRVDRRTGVWKEGADRPTTRRP |
| 137 | MTVGIYIRVSTEEQAREGFSISAQREKLKAYCISQDWQDYKFYVDEGKSAKDTNRPYLKLMLDHIQ |
| | QGLINVVLVYRLDRLTRSVKDLYKLLDLFDKNNCIFRSATEVYDTGSATGRLFITLVAAMAQWERE |
| | NLGERVTMGQVEKARQGQYSAPAPFGFKKQDETLVKDKKQGYILMDMIDKVKKGWSIRQIAKYL |
| | DQSYLPIRGYKWHIATILSILHNPALYGALRWKDELNETSHEGYLTKEEFEELQNILYSRQNFRKRQI |
| | ESAHIFQMKLVCPQCGNRLGCERSVYFRKKDQKNVESLHYRCQSCALNERPSISVSEKKLEKALLL |
| | FMKNVKFDLEPVVKEEKNETTEIQNAIVKIERQREKFQKAWASDLMTDEEFTARMSETRKAHENFT |
| | KRLSEIQRATPLPIDIKKAKKLVNEFKINWAYLNTEEKREFVQSFIEKIEFTKKDQNPHILNVSFY |
| 138 | MSTITKIQSYQRDVKQLRVAAYCRVSTNNIEQLESLENQREHYQKYISNQPNWQLAKIYYDEGISGT |
| | KLTKRDALKELLTDCHNHQIDLVITKSISRLSRNTTDCLRIVRELQQLNIPIIFEKEHINTGEMASELFL |
| | SIFSSLAQDESHSTAGNLRWAIRQRFASGKFHVSSAPYGYSIKDGNLVINHTEAKTVRQVFQRFLSGI |
| | SASQIAKKLNQKQVPTKRGGQWRSNTVINILRNINYTGGMLCQKTYRDDQYHRHFNQGEITQYLIE |
| | DHHPSLINHRSYHRAQVLIKEAAQKHHIEVGSHKYQQHYLFSGKITCGYCGTVFKRQTRPHKICWA |
| | CQQHLKSAQQCPVKAVSEKSLEAAFCNMINELVYSEKFLLRPLLEGLKEEANANSDGQLISLTKQIK |
| | TNDHKAETLTELMHASLLDKAIYVNQTAKLEQDTYQCREKIKQLNGQNTDSANNFEDVRALLRW |
| | CQQGQMLTEFDGTLFQEFVRQVVVNSSNEATFNLKCGLSLPEKLNKNATIDGHFYRDIIKQRYNDPI |
| | KQTEYLYSIIESEGDLIG |
| 139 | MGKVRIIPAHQQKGNSVQPQQSRQPFEQLRVAAYCRVSTDYDEQASSYETQVVHYKELIQKEPTW |
| | EFAGIYADDGISGTNTKKREQFNQMIAACKAGKIDLIVTKSISRFARNTIDCLKYIRDLKAINVAIFFE |
| | KENINTMDAKGEVLITIMASLAQQESESLSQNVKMGIQYRYQQGKIFVNHNHFLGYTKDAQGNLVI |
| | EPAEAKIIKRIFYSYLNGMSMKQIADSLKADGILTGGKTKNWQSSGVSRILKNEKYMGDALLQKTY |
| | TVDFLNKKRVKNNGIMPQYYVENDHPAIIPKPVFMQVQQLIKQRQNGITTKNGKHRRLNGKYCFS |
| | QRVFCGKCGDIFQRNMWYWPEKVAVWRCASRIKRSKSGRRCMIRNVKEPLLKEATVQAFNQLIEG |
| | HKLADKQIKANIMKVIKNSKGPTLDQLDKQLEEVQMKLIQAANQHQDCDALTQQIMDLRKQKEK |
| | VQSRETDQQAKLHNLDEINKLVELHKYGLVDFDEQLVRRLVEKITIFQRYMEFTFKDGEVIRVNM |
| 140 | MTTPLRGLSVLRLSVLTDETTSPERQRTANHDAGAALGIDFSDREAVDLGVSASKTTPFERPELGA |
| | WLKRPDDFDALVFWRFDRAVRSMDDMHELSKWARDHRKMIVIAEGPGGRLVLDFRNPLDPMAQ |
| | LMVTLFAFAAQFEAQSIRERVLGAQAAMRTMPLRWRGSKPPYGYMPAPLESGGMTLVQDEKAVV |
| | VIERAIKELKNGKTLSAICHELNEAGIPSPRDHWSLVQGRKKGGGVGNSVGERIKKESFKWRHGAL |
| | KKLLTSESLLGWKMTRSGPVRDDEGAPVMATREPILTREEFDAVGALIIEANEDGTKWERRDSTAL |
| | LLRVILCDGCGQHMFVGNPSANSKGISAVYKCGAWGRGEKCPEPASVKLEWAEDYVRERFLRSVG |
| | GMRLTETRRIPGYDPQPEIDATTAEYEAHMREQGQQKSKAAQAAWKRRADALDARLAELESREA |
| | RPARVEIVQLGMTIADAWRDADDKERRDMLREAGVTVRIKRAKRGRTFKLNEDRVKWHMANEFF |
| | AQGAEELEAIARDEEHANGSQ |
| 141 | MASHSSWEIHPDLAAALASGKTVEEWLDGRTPVVSYARISVDLQKVKAIGVARQHGMHCDPAAK |
| | EQGSAVVYRYTDNDLTAADPDVQRPAFLQMVRDLRARQTAEGIAIRGILAVEEERVVRLPEDYLK |
| | LYRALTVEEDAVLYYTDKRQLVDVYAEVEQTRGLMSSSMGETEVRKVKRRAKRSTKDRAAEGKY |
| | TGGARRFGWLGADKDLGRTQNEKLDPNESVWLRNMIDMKLCGKGWHTIAVWLISESIATVRGGE |
| | WTSTGVKSLLTNPAICGYRILNGELVLDPGTGEPKVGNWETIATPEEWHQICEMAWPGGKLAKTK |
| | KPKGKKRARKHLSTGILRCGWIPKSDPKEDMCLHSMVGRPPHGNHKWGNYVCNGTDCRKVSRR |
| | MDKIDRIVEGIVVRTLKDQFATLAPEEKTWHGQYTLERLTARRQELKAAYKAEHISMADYLEFIDP |
| | LDAQIKESQADRDAFYAEQAAKNFLAGFTEERWHDFDLEQKQTAIGTVLQAVIVHPLPEGRSRKAP |
| | FDPSLIEIVFKNPH |
| 142 | MKAAIYIRVSTQEQIENYSIQAQTEKLTALCRSKDWDVYDTFIDGGYSGSNMNRPALNEMLSKLHE |
| | IDAVVVYRLDRLSRSQRDTITLIEEYFLKNNVEFVSLSETLDTSSPFGRAMIGILSVFAQLERETIRDR |
| | MVMGKIKRVESGLPLTTAKGRTYGYDVVDTKLYINEEEAQHLQLIYDIFEEEKSITFLQKRLKKLGF |
| | KVKSYSSYNKWLMNDLYIGYVSYSDKVHAKGIHEPIISEDQFYRVKEIFSRMGKNPNMNKESSSLL |
| | NNLIVCEKCGLGYVHRAKDTVSRGKKYHYRYYSCKTYKHTHELEKCGNKIWRADKLEEIIISRVK |
| | NYSFATRNLDKEDELDSITEKLKTEHSKKKRLFDLYINGSYEVAELDKMMADIDAQINYYDSQIEA |
| | NKELKRNKKVQESLAELATVDFDSLEFREKQIYLKSIINKIYIDGEQVTIEWI |
| 143 | MIQAFSYVRFSTKSQATGTSLERQLNASKLFCQQHNLELSSKGYNDLGISGFKNVKRPELDQMLEAI |
| | QSGVIPSGSYILIEAIDRLSRKGISHTQDVLKSILLHDIKVAFVGEDAKTLAGQILNKNSLNDLSSVILV |
| | ALAADLAHKESLRKSKLIKAAKAIIREKAQQGKKIRGHTMFWIDWSESNNKFVLNDKKSIIKEIVKL |
| | RLAGNGPRKIATVLNEQQIPSPSGKQWNHMTVKVALRSPTLYGAYQTHQIIEGKAVPDILIKDHYPA |
| | ITNYETYLQLQSDSSKANKGKPSKANPFSGILKCSCGHGMNFSKKVMVYKDKPHEYEYHFCSASTE |
| | GRCPNKKRIRDLVPLLTSLMDKLTIKQTTKKNLNLEEIKLKEQKIEKLNLMLLEMDNPPLSVLKTIQ |
| | KLEEELNLLLKTTDSPDVSQNDVESLSSINDAQEYNMHLKRIVRKIEVHQLDTTGKNLRIKVLKTDG |
| | HSQNFLIKSGEVLFKSDTEQMKNLLKTMKEA |
| 144 | MAYAVYVRVSSDKDEQVSSVENQIDICRYWLENNGFEWDKNAVYFDDGISGTAWLERHAMQLIL |
| | AKARKKELDTVVFKSIHRLARDLKDALEIKEILLGHGVRLITIEEGYDSHYEGKNDMKFEMYAMFA |
| | SQLPKTLSVSVTAALAAKVRRGGYTGGFVPYGYEIVDDKYAINEEEAELVREIFELYAQGFGYIKIS |
| | NIINDQGKRTRKGAPWTYSTLCKMIKNPTYKGDYTMQKYGTVKVNGKKKKVINPEEKWVVFENH |
| | HPAIVSRELWDKVNNKDPNKFQKKRRISTTNELRGITFCAHCGTAMSKRNNVRVNKNGTVKEYSY |
| | MICDWSRVTARRECVKHVPIHYKDLRALVLSKLKEKESVLDKEFYSDEDQLDVKLKKLNRDIKDL |
| | KFKRERLLDLYLEDERIDKDTFTIRDAKLEKEIELKELEMRKANNIELQMKERQEIRDAFALLEESK |
| | DLNSAFKKLIKRIEVAQDGAVDIHYRFAE |
| 145 | MGKNGARVYSYLRFSDPRQATGSSADRQLAYASAWASKHGMELDATLTLRDEGLSAYHETHVK |
| | QGALGAFLRAVDEGRIPAGSVLIVEGLDRLSRAEPLLAQAQLGQIVNAGITVVTASDGREYNREGL |
| | KAEPMNLVYSLLVMIRAHEESDTKSKRVKAAVRRQCEAWVAGSYRGRIVSGKDPQWLAWDGDS |
| | WQFIPERVEAVRFALDAYRSGIGAARLVRLMHEKGMVLSDWGIAAQQVYRLVRLPALRGAKRISI |
| | DGEDFMLEDYYPRLLSDEEFSELETLVGQRYRRRGKDEIVGIVTGIGITRCGYCGTALVAQNLMQR |
| | VKADGSLEDGHRRLHCVSYSKNGGCNGGSCSSVPIERAVLAYCSDQMNLQRLLEPSSAGEDLRPR |
| | LVEAQKGVAEIERQLERVTDALLADDSGAAPLAFVRKARELEEDLERRRSAVRALEQELVAKSAS |
| | APAAGASKWAELAERAKSMADVDAREQARQLVMDTFETLVVYMRGVIPNPKGRYIDVMMKSRA |
| | GQTRWIRVDRRTGVWKEGADRPTTRRP |
| 146 | MRSESTSAFGQPNDINPILLLSDTATPGSMAIKAKVYSYLRFSDPKQAAGSSADRQMEYARRWAAE |
| | HGMTLDSELSMQDAGLSAYHQRHVTRGALGLFLQAIDDARIPAGSVLVVEGLDRLSRAEPIQAQA |
| | QLAQIINAGITVVTASDGREYNREGLKAQPMDLVYSLLVMIRAHEESDTKSKRVRAAIHRQCQGW |
| | MAGTWHGLVRNGKDPHWLRLVGQAYEIVPERGEAVRTAVSMFRQGHGAVRIMRSLADSGLQITN |
| | GGNPSQQLYRIVRNRALIGEKVLAVDGQEYRLAGYYPPLLSPAEFADLQHLTAQRSRHKGTGEIPG |
| | LITGMRIAFCGYCGAAMVSQNLMNRGRQEDGRPQNGHRRLICVSNSQGGGCPVAGSCSVVPIEHA |
| | LLTFCADQMNLSRLLDFGNRANGIAGQLSIARVQVSDTTARIDKITDALLASDAGQAPAAFLRRAR |
| | ELESELAEQQKRVEALEHELAAVALSPEPAAAKAWAGLVEGVEALDHDARIKARQLVADTFDRIV |
| | VFHRGRTPEHSRSWKGTIDLLLMAKRGGARLLHIDRQTGGWKAGEEIDTIQIPLPPGVAEATSQSEA |
| | LPGLVSR |
| 147 | MKCAIYRRVSTDEQAEKGFSLENQLLRLQAFADSQGWEIVADYMDDGYSGKNTDRPALKKMFAE |
| | IDNFDVILVYKLDRFTRSVRDLNDMLETIKGHDIAFKSVTEAIDTTTATGRMILNMMGTTAQWERE |
| | MISERIKDVLGKLAEQGIFPKGKPTYGYKIKNGVISIDEKEAEVVKLIFEKSKTLGQHAVSKYLRDN |
| | GIYTPSGSTWMSGGIGRIIRNPFYYGEMKVNGKLIAIKNEGYKPLISKEEFDLVNRISKSRNIKNPKR |
| | KSDIIYPFSGIALCPRCNKPLRGDRSKVGGKYYTYYRCINTREGRCTMKRIRTQVIDNAFSEYVAGA |
| | FNEANIQIDNKDERNALERKIEALKSKIDRLKELYIDGDITKVRYKEQTEAINSEINSTQDKMLSLDD |
| | GKITEKAIEKAKELDKVWLLLDDKTKDESLRSVFDTITLEETERGIIITGHSFL |
| 148 | MMDRNKVAIYVRVSTQGQVDDGYSLDEQVDLLTNYCKLKEWTLYDVYVDPGISGKNMHRPEIER |
| | LTRDAKRKLFDIVLIYDLKRLGRSQKENIVLVEDVFNPNGIRLVSFTENFDASTPVGKMVFGMLSAY |
| | AELDRANIAERMMMGKIGRAKAGKAMSWGMPPFAYDYNKETGDLELDEVKAPIVEMIYSEFLKG |
| | ASVNKIVQKLNSMSYHGKNHEWKHHAVTVIIDNPVYCGMMKYMGQTYQAKHTPIIDKKTFELAQ |
| | LERKKRLSKYHDADWLGPFQRKYIGSKICYCGLCGAHLKSEKDKKNKLTGIRSISFFCPNTRSRGTG |
| | ECTNPRFKQSVLEGYILNEVAKLQQNPEKLKDIKPAEDNELHNKIATYEKKIKQNSSKLSKLNDLYL |
| | NDLISLDDLKQQSKSLLNENEFMEEQIKLLSATTREDELRKKIDTFLAFPDILTADYDTQKQAVELVI |
| | SRVEATKEGIDIFFNF |
| 149 | MKAVVTKKRCAVYTRVSTDERLDQSFNSLDAQREAGQAYIVSQRAEGWLPVGDDYDDGGYSGG |
| | NMERPALKRLLADIVADQIDIVVVYKIDRLTRSLTDFAKLVEVFERHKVSFVSVTQQFNTTTSMGR |
| | LMLNILLSFAQFEREVTGERIRDKIAASKRKGLWMGGYTPLGYEIKDRKLVIEEKDAEIIRRIFTRFTE |
| | LRSITDVVRELALEGLTTKPNRLKDGRVRNGTPMDKKYISKLLRNPIYVGEIRHKGTVFAGQHEPIIT |
| | RQLWDRVQGILAEDAYERMGKTQTRHKTDALLRGLMYGPDGGKYHITYSKKPSGKKYRYYIPKA |
| | DSRYGYRSSATGMIPADQIEEVVVNLLVGALQSPESIQGVWNTVRDKYPEIDEPTTVLAMRRLGEV |
| | WKQLFPAEQVRLVNLLIERVQLLSDGVDIVWRESGWRELAGELQADSIGGELLEMEMTP |
| 150 | MKKITKIEGNQDYIFKPKTRVVAYCRVSTDSDEQLVSLQAQKAHYETYIKANPEWEYAGLYYDEGI |
| | SGTKKENRSGLLRMLSDCETRSIDLIITKSISRFARNTTDCLEMVRKLMDLGVHIYFEKENINTGSME |
| | SELMLSILSGLAESESISISENTKWAIQRRFQNGTFKISYPPYGYQNIDGRMIVNPKQAEIVKYIFAEV |
| | LSGKGTQKIADDLNRKGIPSKRGGRWTATTIRGILTNEKYTGDVILQKTYTDSRFNRHTNYGEKNM |
| | YLVENHHEAIISHEDFEAVEAILNQRAKEKGIEKRNSKYLNRYSFSGKIICSECGSTFKRRIHSSGRRE |
| | YIAWCCSKHISHITECSMQFIRDEDIKTAFVTMMNKLIFGHKFILRPLLNGLRSQNNAESFRRIEELET |
| | KIENNMEQSQMLTGLMAKGYLEPAMFNKEKNSLEAERESLFAEKEQLTHSVNGIFTKVEEVDRLL |
| | KFTTKSKMLTAYEDELFKNYVEKIIVFSREVVGFVLKCGITLKERLVN |
| 151 | MKAAIYIRVSTQEQIENYSIQAQTEKLTALCRSKDWDVYDIFIDGGYSGSNMNRPALNEMLSKLHEI |
| | DAVVVYRLDRLSRSQRDTITLIEEYFLKNNVEFVSLSETLDTSSPFGRAMIGILSVFAQLERETIRDR |
| | MVMGKIKRIEAGLPLTTAKGRTFGYDVIDTKLYINEEEAKQLQMIYDIFEEEKSITTLQKRLKKLGF |
| | KVKSYSSYNNWLTNDLYCGYVSYADKVHTKGVHEPIISEEQFYRVQEIFSRMGKNPNMNRDSASL |
| | LNNLVVCGKCGLGYVHRRKDTVSRGKKYHYRYYSCKTYKHTHELEKCGNKIWRADKLEELIIDR |
| | VNNYSFASRNVDKEDELDSLNEKLKTEHTKKKRLFDLYISGSYEVSELDAMMSDIDAQINYYEAQI |
| | EANEELKKNKKIQENLADLATVDFDSLEFREKQLYLKSLINKIYIDGEQVTIEWL |
| 152 | MSLMDENTQKNVGIYVRVSTEEQAKEGYSISAQKEKLKAYCISQGWNSYKFYIDEGKSAKDIHRPS |
| | LELMLRHIEQGIIDTVLVYRLDRLTRSVRDLYSLLDYFDKYQAVFRSATEVYDTGSATGRLFITLVA |
| | AMAQWERENLGERVKMGQVEKARQGQFSAPAPFGFTKEGESLVKNPEEGEVLLDMIDKIKKGYS |
| | LRELADYLDESDAIPKRGYKWHIASILVILKNPVLYGGFRWAGEILEGAFEGYISKKEFEQLQKMLH |
| | DRQNFKRRETSSIFIFQAKILCPNCGSRLTCERSIYFRKKDNKNVESNHYRCQACALNKKPAIGISEK |
| | KFEKALIEYMQNANFKREPKIPQEKQQDYDKLHQKIISIEKQRKKYQKAWSMELMTDQEFEQLMA |
| | ETKEALQKALAKLEQNDLHPIEKPLNIERAKELAKMFRENWSVLTGEEKRQTVQELIKHIEFEKKD |
| | NKARILDIHFY |
| 153 | MNKICIYLRKSRADEELEKTLGEGETLSKHRKALLKFAKEKKLNIVEIKEEIVSGESLFFRPKMLELL |
| | KEVENKQYTGVLVMDMQRLGRGNMQDQGIILETFKKSNTKIITPMKTYDLSNDFDEEYTEFEAFM |
| | SRKELKMINRRMQGGRVRSVEDGNYIATNPPLGYDIHWIKKSRTLKINAHECEIIKLIFKLYTEGNG |
| | AGSIAEHLNNLGYKTKFNNNFSRSSVLFILKNPIYIGKVTWKKKEIKKSKNPNKTKDTRTRDKSEWI |
| | VVDGKHEPIISMKMWNKAQEILNNKYHIPYQLVNGPANPLAGIVICSKCKFKMVMRKLKGIDRLLC |
| | RNNKCDNISNRYDSTEKAIVQALERYLNEYRINISNKNKTSNIKPYERQVNILEKELAALNEQKLKL |
| | FDFLERGIYDENTFLERSKNIEKRITKTSSGIEKINDIINKEKKVIKEEDVIKFQKLLDGYKNTDDIKLK |
| | NELMKKLVNKVEYTKDKRGETFGIDIFPKLKP |
| 154 | MTVGIYIRVSTEEQVKEGFSISAQKEKLKAYCTAQGWEDFKFYVDEGKSAKDMHRPLLQEMITHIK |
| | KGLIDTVLVYKLDRLTRSVVDLHNLLSIFDEYNCAFKSATEVYDTSSAMGRFFITIISSVAQFERENT |
| | SERVSFGMAEKVRQGEYIPLAPFGYVKGPDGKLIINEAEKEIFLHVVNMVSTGYSLRQTCEYLTNIG |
| | LKTRRSNDVWKVSTLIWMLKNPAVYGAIKWNNEIYENTHEPLIDKTTFDKLANILSIRSKSTTSRRG |
| | HVHHVFKGRLICPQCGKRLSGLRTKYVNKNKETFYNNNYRCATCKEHRRPAIQISEQKIEKAFIDYI |
| | SNYTLNKANISSKKLDNNLRKQEMIQKEIISLQRKREKFQKAWAADLMNDDEFSKLMIDTKMEIDA |
| | AEDRKKEYDVSLFVSPEDIAKRNNILRELKINWTSLSPTEKTDFISMFIEGIEYVKDDENKAVITKISF |
| | L |
| 155 | MKCIVYVRVSTEEQAKHGYSIAAQLEKLEAYCISQGWELTEKYVDEGYSAKDLHRPYFEKMMNKI |
| | KQGNVDILLVYRLDRLTRSVMDLYKILKILDDNNCMFKSATEVYDTTNAMGRLFITLVAAIAQWE |
| | RENLGERVRLGMEKKTKLGIWKGGTPPYGYKIVDKHLVINEKEQDVVKTVIALLSKTLGFYTVAKQ |
| | LTIKGFSTRKGGEWHVDSVRDIANNPVYAGYLTFNQNLKEYKKPPREQTLYEGNHEPIISKDEFWA |
| | LQDILDKRRTFGGKRETSNYYFSSILKCGRCGHSMSGHKSGNKKTYRCSGKKAGKNCSSHIILEDN |
| | LVKKVFHVFDQIVGSINGPTNATEYSFEKVLELENELKSIERILNKQKIMYENDIIGIDELITKSTELRE |
| | REKKINNELKNIKQNTPKNQKEIEYLTKNIESLWQHANDYERKQMITMIFSRIVIDTEDEYKRGSGN |
| | SREIIIVSAE |
| 156 | MKGESELDKKAAIYIRVSTQEQATEGYSIQAQTDRLTKYVEAKDFILYKKYIDAGYSASKLERPAM |
| | QELIQDVQSKKVDVIIVYKLDRLSRSQKDTMYLIEDIFRPNDVELISMQESFDTSTAFGSATVGMLS |
| | VFAQLERKSISERMITGRVERAKKGFYHTGGQDRPPAGYQFNSDNQLIINEYEAAAIKDLFRLYND |
| | GLGKSSISEYLKKNYPGKNKWLPSSIDRMLKNSLYIGKVKFSGAEYDGIHEPIIDEVTFYKTQKEIAR |
| | RKQSNTKRYNYVALLGGLCECGICGAKMANRRAVGRKGKVYRYYRCYSKKGSPKHMMKTDGCS |
| | SKAQQQFIIDEAVINNLKNIDVEAELKRRSAPQTNTSLISSQIESIDKQINKLIDLFQVDSMPLDVISEK |
| | IDKLNKEKQSMEKLLERKNKLDKTELQHRFDVLKSFDWDNSSIESKRVVIEMLVQKVIIHDNSIEIIL |
| | VE |
| 157 | MNYERSYLRSCQVSTLEQKEHGYSIEEQERKLKSFCEINDWSVSDVFIDAGFSGAKRDRPELQRMM |
| | NDIKRFDLVLVYKLDRLTRNVRDLLDLLEVFEQNNVAFRSATEVYDTSTAMGRLFVTLVGAMAE |
| | WERETIRERVMMGKRAAIKQGMILTPPPFYYDRVDNTYIPNDYKKVVLWAYDEVMKGNSSKAIA |
| | RKLNDSDIPPPNGKRWEDRTITRALRNPITRGHYTWGDVFIENSHEPIITEEMYQQIKERLEERINTKI |
| | VSHVSVFRGKFICPRCGGTLTLNTVTRKRKKGYVTYKTYYCNTCKAKKQSFGFSENEALRVFRDY |
| | LSKLDLEKYEVKTKQKDDVVTIDIDKIMEQRKRYHKLYAKGLMKEEELFGLIKETDETIAEYEKQK |
| | ELVPRKSLDIDKIKKFKNALLESWEIFSLEDKADFIKMAIKSIDIDYVKLKNRHSIKINDIEFY |
| 158 | MKVAIYTRVSTAEQNLNGFSIHEQRKKLISFCEINEWKEYEVFTDGGFSGGSTKRPALQDLFSRLTQ |
| | FDLVLVYKLDRLTRNVRDLLEMLERFEKYNVSFKSATEVFDTTTAIGKLFITIVGAMAEWERETIRE |
| | RSLFGSRAAVESGKYIREQPFVYDNIEGKLVPNENTKYIEYIVKKFKEGNSANEIARLLNSKKKPSKI |
| | KNWNRQTIIRLIKNPVLRGHTKFGDIFMENTHEPVLSDDDYHKVINAIENKTHKSKSKHNAIFRGVL |
| | KCPQCNGNLHLYAGTIRPKNGRSYNVRRYTCDKCHRDKYSRNISFNESEIENKFIEELEKMDLTRFE |
| | IHKPKKVEINIESDKKRIKEQRTKLLRAYTMGYVEEEEFKIIMDETQRQLEDIKREENKETVQEIDEK |
| | QIKSIGNFIIEGWKTLTIKEKEKLILSSVDKIDIEFIPREKNNNSNTNTVNIKKVHFIF |
| 159 | MNYERSYLRSCQVSTLEQKEHGYSIEEQERKLKSFCEINDWSVSDVFIDAGFSGAKRERPELQRMM |
| | KDIKRFDLVLVYKLDRLTRNVRDLLDLLEIFEQNNVAFRSATEVYDTSTAMGRLFVTLVGAMAEW |
| | ERETIRERVMMGKRAAIKQGMILTPPPFYYDRVDNTYIPNDYKKVVLWAYDEVMKGNSSKAIARK |
| | LNDSDIPPPNGKRWEDRTITRALRSPITRGHYTWGDVFIENSHEPIITEEMYQQIKERLEERINTKIVS |
| | HVSVFRGKFICPRCGGTLTLNTTTRKRKKGYVTYKTYYCNTCKGKKKSFGFAENEALRVFRDYLS |
| | KLDLEKYKVKTKQKDDVVTIDIDKIMEQRKRYHKLYAKGLMQEEELFELIKETDETVAEYEKQKE |
| | LVPRKSLDIDKIKKFKNALLESWEIFSLEDKADFIKMAIKSIDIEYVKLKNRHSIEIKDIEFY |
| 160 | MNVAIYCRVSTLEQKEHGYSIEEQERKLKSFCEINDWNVADVFVDAGFSGAKRDRPELQRMMNDI |
| | KRFDLVLVYKLDRLTRNVRDLLDLLEIFEQNNVAFRSATEVYDTSTAMGRLFVTLVGAMAEWERE |
| | TIRERTQMGKLAALKKGIMLTTPPFYYDRVDNKFVPNKYKEVVLFAYEEALKGKSAKSIARKLNN |
| | SDIPPPNNRKWEDRSITRALRSPFTRGHFEWGGVYLENNHEPIITEEMYEKVKDRLEERTNTKKIKH |
| | VSIFRSKLVCPTCHNKLTMNTHKVTLKDRVYYNKHYYCNNCKETPNLKPVYIRAEEVERVFYDHL |
| | QHQDLTQYDIVEDKEEKEVAIDINKVMQQRKRYHKLYANGLMNEDELAELIEETDIAIEEYKKQSE |
| | NKEVKQYDTEDIKQYKNLLLEMWDISSDEEKAEFIQMAIKNIFIEYVLGKNDNKKKRRSLKIKDIEF |
| | Y |
| 161 | MITTNKVAIYVRVSTTSQAEEGYSIEEQKAKLSSYCDIKDWSVYKIYTDGGFSGSNTDRPALEGLIK |
| | DAKKRKFDTVLVYKLDRLSRSQKDTLYLIEDIFIKNNIAFLSLQENFDTSTPFGKAMIGLLSVFAQLE |
| | REQIKERMQLGKLGRAKAGKSMMWAKTSYGYDYHRETGTITINPAQALAVKFIFESYIRGRSITKL |
| | RDDLNEKYPKHVPWSYRAVRAILDNPVYCGFNQFKGEIYPGNHEPIITEEVYNKTKEELKIRQRTAA |
| | ENVNPRPFQAKYILSGIGQCGYCGAPLKIILGVKRKDGSRFKKYECHQRHPRTLRGITTYNDNKKC |
| | DSGFYYKDDLEAYVLTEISKLQDDAGYLDKIFSEDSAETIDRKSYKKQIEELSKKLSRLNDLYIDDRI |
| | TLEELQNKSTEFISMRATLETELENDPALGKDKRKADMRELLNAEKVFSMDYEGQKVLVRGLINK |
| | VKVTAEDIIINWKI |
| 162 | MKAAIYIRVSTQEQIENYSIQAQTEKLTALCRSKDWDVYDIFIDGGYSGSNMNRPALNEMLSKLHEI |
| | DAVVVYRLDRLSRSQRDTITLIEEYFLKNNVEFVSLSETLDTSSPFGRAMIGILSVFAQLERETIRDR |
| | MVMGKIKRVESGLPLTTAKGRTYGYDVVDTKLYINEEEAQHLQLIYDIFEEEKSITFLQKRLKKLGF |
| | KVKSYSSYNKWLMNDLYIGYVSYSDKVHAKGIHEPIISEDQFYRVQEIFSRMGKNPNMNKESSSLL |
| | NNLIVCEKCGLGYVHRAKDTVSRGKKYHYRYYSCKTYKHTHELEKCGNKIWRADKLEEIIISRVK |
| | NYSFATRNLDKEDELDSITEKLKTEHSKKKRLFDLYINGSYEVAELDKMMADIDAQINYYDSQIEA |
| | NKELKRNKKVQESLAELATVDFDSLEFREKQIYLKSIINKIYIDGEQVTIEWI |
| 163 | MNVAIYCRVSSQEQANEGYSIHEQERKLKSFCEVNNWKNYKVFVDAGVSGGTINRPAFNNLLANL |
| | DKFDLVLVYKLDRLTRSVRDLLSLLETFEEHGVSFRSATEVFDTTSAIGKLFITIVGAMAEWERSTIR |
| | ERSLFGSHAAVREGNYIRVAPFCYDNIDGKLVPNEHKKVIEYIVKKLLEGVTATEIARRLNNANNYP |
| | PTIKNWSKTTVIRLVNNPVMRGHTKHGDLFIENTHEPIITEHNYKRISERLSSRVNYKKQTHTSVFRG |
| | VLECPQCGHKLHYFKSKLKNKSKTYYSEGYRCDYCRTDKTARNIAITFSEIEREFIEYMSNIRLSDN |
| | YGIEVEPKNEVIKIDINKIMRKRSRFQEAYGDGLMTKEEFKQKMKETQKLIDEYEEAESKNDVDDHI |
| | TKEQVQAVQNLFRHIWDSPNVTREDKEEFVRQSIKKIDFDFIPKSKVNKTPNTLKINNIDLHF |
| 164 | MNVAIYCRVSSQEQANEGYSIHEQERKLKSFCEVNNWKNYKVFVDAGVSGGTINRPAFNNLLANL |
| | DKFDLVLVYKLDRLTRSVRDLLSLLETFEEHGVSFRSATEVFDTTSAIGKLFITIVGAMAEWERSTIR |
| | ERSLFGSHAAVREGNYIRVAPFCYDNIDGKLVPNEHKKVVEYIVKKLLEGVTATEIARRLNNANNY |
| | PPTIKNWSKTTVIRLVNNPVMRGHTKHGDLFIENTHEPIITEHNYKRISERLSSRVNYKKQTHTSVFR |
| | GVLECPQCGHKLHYFKSKLKNKNKTYYSEGYRCDYCRTDKTARNIAITFSEIEREFIEYMSNIRLSD |
| | NYGIEVEPKNEVIKIDINKIMRKRSRFQEAYGDGLMTKEEFKQKMKETQKLIDEYEEAESKNDVDD |
| | HITKEQVQAVQNLFRHIWDSPNVTREDKEEFVRQSIKKIDFDFIPKSKVNKTPNTLKINNIDLHF |
| 165 | MKNKIAIYVRVSTTKESQKDSPEHQKWACIEHCKQIDLDTADLIIYEDRDTGTSIVARPQIQEMISDA |
| | QKGLFNTILFSSLSRFSRDALDSISLKRIFVNALGIRVISIEDFYDSQIEDNEMLFGIVSVVNQKLSEQIS |
| | VASKRGIKQSAAKGNFIGNIAPYGYQKVNIEGRKTLIVDIEKAKVVREIFDLYVNKKMGEKEITKHL |
| | NENAIPSAKGGTWGITSVQRILQNEIYTGYNVYGKYEIKKVYTNLKNIGDRKRKLVKKDQELWQK |
| | SEKRTHPEIISQELYKKAQEIRQIRGGGKRGGRRKYVNVFAKIIYCKHCGSAMVTASCKKSDKYRY |
| | LICSKRRRHGASGCPNDKWIPYYDFRDEVISWVVEKLKK |
| 166 | MARTKKATAPAIYASPRVYSYLRFSNAKQASGASIARQLDYAVKWAEQHGMELDTSLTLKDEGLS |
| | AFHEKHIEKGNFGVFLKAIEDGLIPPGSVLIVESLDRLSRAEPIIAQAQLYGILIAGIEVVTAADNTRIS |
| | LESVKKNPGILFLALGVSMRANEESERKKDRILDAAHRNAQAWQAGTSRKRAAVGKDPGWVKYN |
| | AKTNEYELLPEFVTPLMAMLGYFRAGASTRRCFAMLHEAGIPLPPPKLDLHGKLKKTRMGNVISGL |
| | ANTTRLYDIMSNRALIGEKTIVLGKSQYHDAQTYVLSGYYPPLMTEAEFEELQQMRKQGGRVANH |
| | QSRIVGIINGVGITKCMRCRSAMAGQNVLSRSRRADGKPQDGHRRLICTGVTKAKNLCTESSVSIVP |
| | IERAIMAYCSDQMNLTALFTEQEDQSRNLNGQLALARAAVAQTEAAMQKLLDAIEAAGDDTPAM |
| | FIQRARKREIELKTQQQAVADLEYKIESAHRASRPAMAEVWAKLRNGVEQLDPAARTKARLLVVD |
| | TFKRIEIKRATDRGQDLIEIRLESKQNVRRGFLIDRKTGAFYRGDHVENESIIAKPTTRPTRARRVKA |
| | AA |
| 167 | MLKIAIYSRKSVETDTGESIKNQIAICKQYFQRQNEECKFEIFEDEGFSGGNINRPDFKRMMQLVKIK |
| | QFDVVAVYKVDRIARNIVDFVNVFDELDKLNVKLVSVTEGFDPSTPIGKMMMMLLASFAEMERM |
| | NIAQRVKDNMRELAKLGRWSGGTAPSGYSVQKVKENGKEVSYLKKEKDADNIKLIFQKYASGYT |
| | AFEIHKYFKLKGFTYNPKTIYGILTNPTYLEATEESIKYLENKGYTVYGEPNGCGFLPYNRRPRYKGI |
| | KAWKDKSMMVGVSRHEPAVDLNLWIAVQSQLEKKTVAPHPHESKFTFLTGGIMKCRCGAGMGV |
| | SPGRIRSDGTRVYYFTCSGKRYRQNGCSNLSLRVDWAESKVKTFLEKMRDKETLTKYYNSNKKKS |
| | NVDRDIKSINKKIASNKKAVDSLVDKLILLSNDAAKPLAERIEDITQESNALKEELLKLEREKLFNSN |
| | DRLNIDLIHKAIIQFLDTDSLEEKKKFAKDIFDKITWDSASKELLFFLQM |
| 168 | MTVGIYIRVSTQEQASEGHSIDSQKERLASYCNIQGWEDYRFYVEEGISGKSTNRPKLQLLMDHIEK |
| | SQINTLLVYRLDRLTRSVIDLHKLLNFLNLHNCALKSATETYDTTTANGRMFMGIVALLAQWESEN |
| | MSERIKLNLEHKVLVEGERVGAVPYGFDLSDDEKLIKNEKSPILLDMVKKVESGWSANRVANYLN |
| | LTNNDRNWTANAIFRLLRNPAIYGATKWNDKIAEKTHEGIIDKERFVRLQQIFSDRSIHHRRDVKST |
| | YIFQGVLHCPNCSNKLSVNRFNRKRKDGSEYHGVIYRCQPCAKQNKMNFTIGEARFSKALIEYMAR |
| | VEFQPQEEEITSTKSGRDIHQSQLQQIERKRGKYQKAWASDLISDTEFEKLMNETRYAYDECKKKL |
| | HECEEPIKQDIERLKEIVFVFNETFNDLTQDEKKEFISRFIRNIRYTTQEQQPIRTDQSKSRKGKPKVII |
| | TEVEFY |
| 169 | MRAAIYTRVSTFDQVNGYSLDMQAHLAKQYCRDKGIDIYDVYCDEITGAKFDRPQLQRMLTDIVS |
| | KKIDLVVIHKLDRLSRSLKDTFVIVEDYLIANDVELVSLSEAIDTTTPIGKMMMGQFALYAQYERDV |
| | IRERMIMGKYGRAMTGKAMSWAPGYTPLGYDYKDGLYIPNNDKIIVVEIFDELYKGTKPKSLAKK |
| | LTYKGTLNKKWYHTSIKYIARNPVYIGKIKWRGKEFEGNHQPLIAKDFFRAVQEILDEYK |
| 170 | MYYERSYLRSCQVSTLEQKEHGYSIEEQERKLKSFCEINDWTVTDTFIDAGFSGAKRDRPELQRLM |
| | NDINKFDLVLVYKLDRLTRNVRDLLDLLEIFEQNNVAFRSATEVYDTSTAMGRLFVTLVGAMAEW |
| | ERETIRERTQMGKLAALKKGIMLTTPPFYYDRVDNKFVPNKYKEVVLFAYEEALKGKSAKSIARKL |
| | NNSDIPPPNNRKWEDRSITRALRSPFTRGHFEWGGVYLENNHEPIITEEMYNKIKDRLNERVNTKVI |
| | AHTSVFRGKLTCPTCGAKLTMNTNKKKTRNGYTTHKNYYCNNCKITPNLKPVYIKEREILRVFYD |
| | YLLNLNLEKYEIEEKQSEPEITVDIHKVMEQRKRYHKLYANGLMQEDELFDLIKETDEAIKEYESQT |
| | KNKVEKQFDIEDVKKYKKLLLEMWNVSTLEDKAEFVQMAIKSIEFDYIIDDGPPTSRKHSLKINQIIF |
| | Y |
| 171 | MYYGRSYLRSCQVSTLEQKEHGYSIEEQERKLKQFCEINDWTVSDTFIDAGFSGAKRDRPELQRLM |
| | NDINKFDLVLVYKLDRLTRNVRDLLDLLEIFEQNNVAFRSATEVYDTSTAMGRLFVTLVGAMAEW |
| | ERETIRERTQMGKLAALKKGIMLTTPPFYYDRVDNKFVPNKYKEVVLFAYEEALKGKSAKSIARKL |
| | NNSDIPPPNNRKWEDRSITRALRSPFTRGHFEWGGVYLENNHEPIITEEMYNKIKDRLNERVNTKVV |
| | AHTSVFRGKLTCPTCGAKLTMNTNRKKTQNGYTTHKNYYCNNCKIMPNLKPVYIKEREVLRVFYD |
| | YLLNLNLEKYEIEEKQSEPEITVDIHKVMEQRKRYHKLYANGLMQEDELFDLIKETDEAIKEYESQT |
| | ENKVEKQFDIEGVKKYKKLLLEMWNVSTLEDKAEFVQMAIKSIEFDYIIDDGPPTGRKHSLKINQIIF |
| | Y |
| 172 | MLRIAIYSRKSVETDTGESIQNQIKLCKEYFKRQDPNCIFEIFEDEGYSGGNINRPSFQRMMELVKIK |
| | QFDIVAVYKIDRIARNIVDFVNTYDELDNIGVKLVSITEGFDPSTPAGKMMMLLLASFAEMERMNIA |
| | QRVKDNMRELAKMGRWSGGTPPKGYTTKKVIENGKKITYLDLIDDEAYIIKDAFKLYAEGYSTYKI |
| | NKHFKEKGIRLPQKTIQNMLNNPTYLISSKESVDFLKNKGYTVYGEPNGFGFLPYNRRPRTKGKKS |
| | WNDKSQFVGVSKHEGIIDLPLWIEVQNKLKERTVDPHPRESNFTFLSGGLLKCSCGSSMFVHPGHT |
| | RKDGSRLYYFRCMKNNGNCSNSKFLRVDYAESSILEFLESISSKEKLTEYQKKKKPRLDFSIEIKNLN |
| | KKIRDNSKAIDNLIDKLMILSNEAGKVVATKIEELTKQNNILKESLLEIERKKLLSGLEDNNLNILYN |
| | EIQNFIQTEDISLRRLKIKNIIKYITYNPQNDSLQVELVD |
| 173 | MATKARVYSYLRFSDPKQAAGSSADRQLEYAKRWAAEHGMTLDAALSMQDEGLSAYHQRHVTK |
| | GALGVFLAAIDEGRIPAGSVLIVEGLDRLSRAEPIQAQAQLAQIINAGITVVTASDGREYNRAGLKA |
| | QPMDLVYSLLVMIRAHEESDTKSKRVRAAIHRQCRGWQDGSWRGVIRNGKDPSWTRLEPETKTFQ |
| | LVPERAEAVKLAIRMFRDGHGAVRIMRTLAEEGLQLTNGGNPAGQLYRILRNRALIGEKVLEIDGE |
| | EYRLAGYYPSLLSAEQFADLQQATEQRAKQKGTGEIPGLITGLRISYCGYCGSAMVAQNLMNRGR |
| | REDGGPQHGHRRLICVGNSQGMGCAVAGSCSVVPIEHAIMSYCADQMNLARLFEGGDRSEALGGR |
| | LAIARARVADTTAKIERITDAMLADDAGDAPAAFMRRAREMEAALAAQQSEVEALEHEMAAIGSS |
| | PTPAVAKAWADLQEGVKALDYDARTKARQLVADTFERISIYHRGTEPEQTRSWKGTIDLVLVAKR |
| | GSARILHVDRQTGEWRGGEEVRDLPDDPVQ |
| 174 | MRCAIYRRVSTDEQAEKGHSLDNQKFRLESFAMSQGWEITGDYVDDGYSGKNMERPALKRMFAD |
| | IDNFDVILVYKLDRFTRSVRDLNDMLETIKGHEIAFKSVTEAIDTTTATGRMILNMMGSTAQWERE |
| | MISERIKDVLGKLAEQGIFPKGKPTYGYKIKNGVISIDEEEAKIVKLIFEKSKTLGQHAVSKYLRDNG |
| | IYTPSGSTWMSGGIGRIIRNPFYYGEMKVNGKLIAIKNEGYTPLISKEEFDLVNRISKSRNMKKTKRK |
| | SNIIYPFSGIALCPRCNKPLRGDRSKIGEKYYTYYRCMNAREGRCTIKRIKTQVIDIAFSEYVSGAFNE |
| | SNIQIDNKDESIALERKIEALKSKVDRLKELYIDGDITKVRYKEQTDAINIEINSMQDKMLSLDDGKI |
| | TEKAIEQAKELEKVWLLLDDKTKDESLRSVFDTITLKETEHGIIITSHSFL |
| 175 | MKLLVTYIRWSTKEQDSGDSLRRQTNLIDAFYSKHKNDYYLLPAHRYVDKGKSGFHQQHKNQGS |
| | DFRRMFENVMSGVIPEGSLIVVENFDRFSRADIDTAIDDVRQILRKGVSILTLGDGELYDKSALTDPV |
| | KLIKHIIIAERAHQESLVKQKRIAQVWNHKTQLARELKKPMGKQAPGWLELSDDGSHYIVDEDKAS |
| | LVNIIYDKRLSGMSMFAICKWLNEQGYPTINQRKVRISKTKKPDGNWSALSVKHILTSRSVLGYLPA |
| | KISTEDRKTVLREEIESFYPQIVTDSKFYAVQQLLEETGKGKTSSGEHWLYVNILKGLIRCKCGLVM |
| | TPTGIRKPVYQGTYRCNGNKESRCSYGTVSRKLLDTQLCSRLFSKLSQLHDEATDTAKLDELQRRL |
| | NIVDSELEKLTETLIQLPNITQIQEALRVKQGEKDELIVQLSREKARVKSVSSLNLSGLDMESVEGRT |
| | EAQIIIKRLVKEIVVSGNEKLVDIYLHNGNMIRGFPLDGKDDHTLTLEEATDEMQPLDDMLIFGEPV |
| | TRIYPAGDMEEVDA |
| 176 | MGKNGARVYSYLRFSDPRQATGSSADRQLAYASAWASKHGMELDATLTLRDEGLSAYHEAHVK |
| | QGALGAFLRAVDEGRIPAGSVLIVEGLDRLSRAEPLLAQAQLGQIVNAGITVVTASDGREYNREGL |
| | KAEPMNLVYSLLVMIRAHEESDTKSKRVKAAVRRQCEAWVAGSYRGRIVSGKDPQWLAWDGDS |
| | WQFIPERVEAVRFALDAYRSGIGAARLVRLMHEKGMVLSDWGIAAQQVYRLVRLPALRGAKRISI |
| | DGENFMLEDYYPRLLSDEEFSELETLVGQRYRRRGKDEIVGIVTGIGITRCGYCGTALVAQNLMQR |
| | VKADGSLEDGHRRLHCVSYSKNGGCNGGSCSSVPIERAVLAYCSDQMNLQRLLEPSSAGEDLRPR |
| | LVEAQKGVAEIERQLERVTDALLADDSGAAPLAFVRKARELEEDLERRRSAVQALEQELVAKSAS |
| | APAAGASKWAELAERAKSMADVDAREQARQLVMDTFETLVVYMRGVIPNPKGRYIDVMMKSRA |
| | GQTRWIRVDRRTGVWKKGADRPTTRRP |
| 177 | MGKNGARVYSYLRFSDPRQATGSSADRQLAYASAWASKHGMELDATLTLRDEGLSAYHETHVK |
| | QGALGAFLRAVDEGRIPVGSVLIVEGLDRLSRAEPLLAQAQLGQIVNAGITVVTASDGREYNREGL |
| | KAEPMNLVYSLLVMIRAHEESDTKSKRVKAAVRRQCEAWVTGSYRGRIVSGKDPQWLAWDGDS |
| | WQFIPERVEAVRFALDAYRSGIGAARLVRLMHEKGMVLSDWGIAAQQVYRLVRLPALRGAKRISI |
| | DGEDFMLEDYYPRLLSDEEFSELETLVGQRYRRRGKDEIVGIVTGIGITRCGYCGTALVAQNLMQR |
| | VKADGSLEDGHRRLHCVSYSKNGGCNGGSCSSVPIERAVLAYCSDQMNLQRLLEPSSAGEDLRLR |
| | LVEAQKGVAEIERQLGRVTDALLADDSGAAPLAFVRKARELEEDLERRRSAVQALEQELVAKSAS |
| | APAAGASKWAELAERAKSMADAEAREQARQLVMDTFETLVVYTRGVIPNPKGRYIDVMMKSRA |
| | GQTRWIRVDRRTGVWKEGADRPTTRRP |
| 178 | MGKNGARVYSYLRFSDPRQATGSSADRQLAYASAWASKHGMELDATLTLRDEGLSAYHEAHVK |
| | QGALGAFLRAVDEGRIPAGSVLIVEGLDRLSRAEPLLAQAQLGQIVNAGITVVTASDGREYNREGL |
| | KAEPMNLVYSLLVMIRAHEESDTKSKRVKAAVRRQCEAWVAGSYRGRIVSGKDPQWLAWDGDS |
| | WQFIPERVEAVRFALDAYRSGIGAARLVRLMHEKGMVLSDWGIAAQQVYRLVRLPALRGAKRISI |
| | DGENFMLEDYYPRLLSDEEFSELETLVGQRYRRRGKDEIVGIVTGIGITRCGYCGTALVAQNLMQR |
| | VKADGSLEDGHRRLHCVSCSKNGGCNGGSCSSVPIERAVLAYCSDQMNLQRLLEPSSAGEDLRPRL |
| | VEAQKGVAEIERQLERVTDALLADDSGAAPLAFVRKARELEEDLERRRSAVQALEQELVAKSASA |
| | PAAGASKWAELAERAKSMADVDAREQARQLVMDTFETLVVYMRGVIPNPKGRYIDVMMKSRAG |
| | QTRWIRVDRRTGVWKKGADRPTTRRP |
| 179 | MAVSRNVTVIPAIKRIGNNKNSESKPKIRVAAYCRVSTDSEEQASSYEIQIEYYTNYIKRNKEWELA |
| | GIFADDGITGTNTKKRDEFNRMIEECMAGNIDMIITKSISRFARNTLDCLKYIRQLKDKNIAVFFEKE |
| | NINTMDSKGEVLLTIMASLAQQESQSLSQNVKLGIQYRYQQGEVQVNHKRFLGYTKDENKQLVID |
| | PEGAKVVKRIYREYLEGASLLQIARGLEADGILTAAGKAKWRPETLKKILQNEKYIGDALLQKTYT |
| | VDFLSKKRVKNNGIVPQYYVENSHEPIIPRELFMQVQEEMVRRANIRGGKGGKKRVYSSKYALSSI |
| | VYCGQCGDIYRRVHWNNRGYKSIVWRCVSRLEEKGSECTAPTINEETLQAAVVKAINELLTNKEPF |
| | LSTLQKNIATVLNEENDNTTDDIDRRLEELQQQLLIQAKSKNDYEDVADEIYRLRELKQNALVENA |
| | DREGKRQRIAEMTDFLNKQSRELEEYDEQLVRRLIEKVTIYEAKLTVEFKSGIEIDEEI |
| 180 | MTVGIYIRVSTDEQVKEGFSISAQKEKLKAYCTAQGWEDFKFYVDEGKSAKDMHRPLLQEMITHIK |
| | KGLIDTVLVYKLDRLTRSVVDLHNLLSIFDEYNCAFKSATEVYDTSSAMGRFFITIISSVAQFERENT |
| | SERVSFGMAEKVRQGEYIPLAPFGYVKGPAGKLIVNEAEKEIFLHVVNMVSTGYSLRQTCEYLTNI |
| | GLKTRRSNDVWKVSTLIWMLKNPAVYGAIKWNNEIYENKHEPLINKATFNKLANILSIRSKSTTSRR |
| | GHVHHVFKGRLICPQCGKRLSGLRTKYVNKNKETFYNNNYRCATCKEHRRPAIQISEQKIEKAFID |
| | YISNYTLNKADISSKKIDNNLRKQEMIQKEIVSLQRKREKFQKAWAADLMSDDEFSKLMIDTKMEI |
| | DVAEDRKKEYDVSLFVSPEDIAKRNNILRELKINWTSLSPTEKTDFISMFIEGIEYVKNDENKAVITKI |
| | RFL |
| 181 | MSKLSKPKVYSYLRFSDPKQAAGSSADRQMEYAARWAAEHEMQLDASLTLRDEGLSAFHQRHIK |
| | QGALGVFLRAVEDGRILPGSVLVVEGLDRLSRAEPIQAQAQLAQIINAGITVVTASDGRRYNRERLK |
| | AQPMDLVYSLLVMIRAHEESDTKSKRVKAAIRRQCEGWVAGTWRGIVRNGKDPHWVRQVENGA |
| | FEFLPERELAIRTMIDLFLAGHGAIEIARILSERELYVSNAGNYSTHMYRIVRNRALIGEKSLTVDGEE |
| | FRLAGYYPALLTPDAFATLQEAMSERGRRKGKGEIPNILTGLSISSCGYCGLALVSQNTAIRPAKGR |
| | AFTRRLGCSGATFNTGCPVGGTCDARIVERALMHYCSDQFNLTRLLEGDDGAARRVAQLAVARQ |
| | RAGEIEMQIQRVTDALLSDDGVAPVAFMRRARELEGELEQQHREIEVLEHQIAASNAHEIPAAAEA |
| | WAQLVDGVLALDYGARMKARQLVADTFRKIVLFQRGFTPFNNAPADRWKRSGTIGLLLVTKRGG |
| | MRLLNIDRKTGQWEAEDNLDLAPHHADEIPLPPTVQGMEC |
| 182 | MSKLSKPKVYSYLRFSDPKQAAGSSADRQMEYAARWAAEHEMQLDASLTLRDEGLSAFHQRHIK |
| | QGALGVFLRAVEDGRILPGSVLVVEGLDRLSRAEPIQAQAQLAQIINAGITVVTASDGRKYNRERLK |
| | AQPMDLVYSLLVMIRAHEESDTKSKRVKAAIRRQCEGWVAGTWRGIVRNGKDPHWVRQVENGA |
| | FEFLPERELAIRTMIDLFLAGHGAIEIARILSERELYVSNAGNYSTHMYRIVRNRALIGEKSLTVDGEE |
| | FRLAGYYPALLTPDAFATLQEAMSERGRRKGKGEIPNILTGLSISSCGYCGLALVSQNTAIRPAKGR |
| | AFTRRLGCSGATFNTGCPVGGTCDARIVERALMHYCSDQFNLTRLLEGDDGAARRVAQLAVARQ |
| | RAGEIEMQIQRVTDALLSDDGVAPVAFMRRARELEGELEQQHREIEVLEHQIAASNAHEIPAAAEA |
| | WAQLVDGVLALDYGARMKARQLVADTFRKIVLFQRGFTPFNNAPADRWKRSGTIGLLLVTKRGG |
| | MRLLNIDRKTGQWEAEDNLDLAPHHADEIPLPPTVQGMEC |
| 183 | MKMKSVLYARVSTEDLEQNNSYIQQQLYQDDRFEIVKIFSDKASGSSVDGRESFLEMLKYVGISKE |
| | GNNYFVEHRTEIECIIVANVSRFSRSVVDARLIIDALHKNNVKVFFVDLNKFSDDADIFLQLNMYLM |
| | IEEQYLRDVSKKVKAGMQRKQSTGYILGSNKIWGYNYVTKDDGKGYLVPHETESLMVKNIFKEYI |
| | TGAGTRTLAKKYKLSSSTILGILKNTKYCGYMGYNLKSDNPTYVKSPFIEPLISTEAFEEVQRIIKGR |
| | CNSESGRGRRIKVRNLTGKIKCECGANYHYKQRETEWCCGREGVEGRTKGCGSPQFNTKLIIPYLE |
| | KNIDNIEKNLEFNLNREIKDINVGSFDRLNQRKEELIRQQDKLLDLYLDEDKLKNISKEMLERRSKLI |
| | KEEIEEVEEKLVILNDMSSHLNNLRRIKVEYKNEIKNIRRLIEEKNLDEIEKLISKIQLETIVNIINFRKE |
| | LRIKEIQFTCFNELYNTNFIFAPEPKKVWDK |
| 184 | MEKVAIYIRVSKKEQSRDKGSDSSLNLQLKKCLDYCKEKDYEVLKVYQDIESGRIDDRKEFNELFE |
| | AISKKIYTKIVFWEISRIARKISTGMKFFEELELYKITFDSISQPYLKDFMTLSIFLAWGTEDLKQMSL |
| | RIKSNLEEKTKAGYFVHGRPATGYIRGENKMIIPDPQKAPYILSIFETYAKNFNLTETARIFNKTRKDI |
| | VEIIDNKIYIGYVPFRKYIQELNQKKRTQVNKKDIKWYKGLHEPIVPLELFEFCQSIREKNIKSRAAY |
| | GDYKPHLLFSSMIYCECGDKMYQQKRNRTYKDNTNYVYYSYSCKNRKHKKSFSARIMDKTIKEMI |
| | LNSKELEDLNNYNSNDIEKSEKKLLKLENNLKLLENERERIINLFQKSYISEDELENKFKDLNTRIQIA |
| | KEKKIEFENTLNIPRNNDIKVLEKLKFIIENYDEEDVIETRKILKMIIKEIRVISFYPLKISILFY |
| 185 | MKTIHKLARPQLPEPPKLKVAAYARASTSSNEQLASLQTQITHYENHIQNNDQWEYVGVYYDEGT |
| | SGTKVEKRDGLHRLIKDAELGKIDLILTKSISSFSRNTVDCLNLVRKLTDIGVTIFFEKENINTGDMES |
| | ELLLSILSSLAESESYSHSENMKWANRKRMAKGIFKTVPPYGYQRKGADFYLIPDEAKVIEQIFKWA |
| | LEGVSAYQVAKRLNEKNIFTRKGSKWQDSGINNILHNIVYTGTMIHQRYFNDDQFRKKKNNGELP |
| | MYRIDNNHPPIISWEDYERVQELITLRANAKGTSKGSQKYSQRYVFTKRIICDKCGCNYKRVHIAGK |
| | GNTKVVKWSCTGHLKNKDGCDALPITDESLKTAYLTMLNKLILGHTIVLEPLINTPVEGKASKQEL |
| | EKLSIEITKIDEKLEVLASLNASGVVSTKTALEEQGRLQMELNKLQEKQHKIMESVNGTSTQRIQLE |
| | QLHQFTKRSEMLTEWDEDLFLRFAELIVVYSRQEVSFELKCGLLLKERLEA |
| 186 | MRKITTLDVTTSSAVKPKQKVAAYIRVSTSNEDQLISLEAQRRHYKTLIEKNVEWQLIDIYSDEGITG |
| | TKKDRRPELIRLISDCEKGKIDFILTKSISRFARNTIDCLELVRKLMDLGVHIYFEKENINTNSMESEL |
| | MLSILSSLAENESVSLSENSKWSIRQRFKRGTYKLSYPPYGYDYIDEQVIVNKKQAQVVKRIFNSVL |
| | EGVGTERIARQLNKEKIPTKRNGKWTGTTIRGIIKNEKYTGDVLLQKTYTDEHFNRKVNQGELDQY |
| | LIENHHEAIITHADIALVANRMLEYQASQKNIAVGSRKYLNRYPFSGKIECAECGDTFKRRIHTSTHS |
| | KYIAWCCSTHIKNKDECSMLFIREERIHQAFITMMNKLKFGYSYVLTSLSKQLETSNQDETYQKITEI |
| | EEQLEVIKDKLNTLIQLMAKGFLEPAIFNEQKIELSQRHMKLKEEREQLLYLINDGSNQLSEVKRLIK |
| | YFKQGKFIDAFDEESFQDIVKKIIVYSPNEIGFHLNCGITLREGVKR |
| 187 | MKRITKIEQDNANALMPKLRVAAYCRVSTASDDQLVSLEAQKTHYESYIKANPEWDFAGVYYDK |
| | GVTGTKTEGRDELLRLISDCENGLVDFIVTKSISRFSRNTLDCLELVRRLLDIGVFVYFEKENLNTQS |
| | MEGELMLSILSGLAESESVSISENNKWSAQKRFQNGTFKVAYPPYGYDNVDGQMVINEEQAEIVR |
| | WMFAQALAGKGAHKIASELNERGVPTRKGGNWTATTVRGLLANEKFTGDILFQKTYTDSQFNRH |
| | HNNGERDRYFMEDHHPAIVSRETFEAVAAVIGQRGKEKGVTRGSKYQNRYPFSGRIVCSECGSTFK |
| | RRIHYSTHQKYIAWCCSRHIEMIEACSMQFIRNDAVEAAFITMMNKLVYGHRTILRPLLDALRGTN |
| | DTGAYHKVAELESRMEEVMERSQVLTGLMTKGYLEPALFNKEKNALEAELENLQRQKDSLSRVL |
| | NGNLAKTEEVSRLLKFAAKAEMASDFDGDLIALKYVDRVVVYSRTEIGFELKCGLTLKERLVR |
| 188 | MKVPVWCYARISTLKQIDGFGIQRQINTINQFLQYVVLDHRLPFTLDVDNVTQMVAEGKSAFRGG |
| | NWKPSTKLGKYRKMVMDGVISDSVLIVENIDRLTRLDPFQAVEIISGLINRGTTILEIETGMTYSRYIP |
| | ESITVLTMQINRANGESKRKSIMMQKSHANRYGKVSKVRPRWFDVVEIDGIKQYRPNETAKAIQR |
| | MYNDYINGIGAAHIVRTYGNTDNGKAWTLVTVLRALSDKRVADDARYPPIIDKELYDSVQALKAA |
| | TNKKGNTHQKNMLNIFSGMSRCPVCNQSIIVKRNSHGNLFTVCLGKRTNKTCEARSISYFALERPLL |
| | TAISGLDFSEVYKHEDKNVLTLRDQWIQNERDIAAFRERLNKASRHEKFAILDELEIMNREQEELTIR |
| | LKSVDVPKDIQLTFDDDKLDLDTNYRIELNNRIKKLIQHINIVREDVSKSSYTIYCTIKYWTDVISHL |
| | VIIDVNIKRTGTGGTNTLTTTLRSVSSLNMDGTVSGNPDSDAWEYWKSFLDGTIGLVDYKK |
| 189 | MRCAIYRRVSTDEQAEKGFSLENQKLRLESFATSQGWEVVEDYVDDGFSGKDTNRPALQRMFSNV |
| | DKFDVILVYKLDRFTRSVKDLNEMLETIKKNEIAFKSATESIDTTTATGRMILNMMGTTAQWERETI |
| | SERIKDVFGKLRENGIFSTGHPPYGYRCSGNKSIEIVEEQAEMVRYIYELSKTMGLFKISVELNRKGI |
| | KTRRNNKFGQSAVKRILHNPFYCGYMEVDNKWVPIKNEGYTPIISEEEFKTTQKILTKRTKAQTRSR |
| | SVSYYPFSGIVLCPECQRAMRGDRAKYGDYYYRYYRCVYGRENINCTNRKRIRAEQVDKAFAEYI |
| | SRSFENTTIKLDSRDIKSDIEYELKHLDSKIERLSDIYIEGDITKSKYNEKMNSLLNEKEKLKKDLTSC |
| | KEHVDAEFVRNQINKLESIWNLIDDKTKSESIRSIFDTIKIKQDKNTVTIMDHTLL |
| 190 | MKAAIYIRVSTQEQVENYSIQAQTEKLTALCRSKDWDVYDIFVDGGYSGSNMNRPALNEMLSKLH |
| | EIDAVVVYRLDRLSRSQKDTITLIEEYFLKNNVEFVSLSETLDTSSPFGRAMIGILSVFAQLERETIRD |
| | RMVMGKIKRVESGLPLTTAKGRTFGYDVVDTKLYVNKEEAQHLQLIYDIFEEEKSITFLQKRLKEL |
| | GFKVKSYSSYNKWLMNDLYIGYVSYSDKVHVKGVHEAIISEEQFYRVQEIFSRMGKNPNMNRDSS |
| | SLLNNLIACEKCGLSFVHRVKDTASRGKKYRYRYYSCKTYKHTHELEKCGNKIWRADKLEEIIIDR |
| | VKNYSFATRNLDKEDELDSINAKLQVEHSKKKRLFDLYMNGSYEVAELDKMMADIDAQINYYNS |
| | QIEANEELKRNKKVQESLAELATVDFDSLEFREKQIYLKSIINKIYISDEQVTIEWI |
| 191 | MKVAIYTRVSTLEQREKGHSIDEQERKLRSFCDINDWTVKDVYVDAGFSGAKRDRPELTRLLDDIS |
| | EFDLVLVYKLDRLTRSVRDLLDLLEVFENNNVAFRSATEVYDTTTAIGRLFVTLVGAMAEWERETI |
| | RERSLMGKRAAIKKGMILTAPPFYYDRVNNTYIPNQYKDVVLDVYNKVKKGYSIAHIARLYNNSD |
| | VKPPNGNEEWTTRMLMHALRNPVTRGHYQWGEIYIEDSHEPIITDEMYNTIIDRLDKHTNTKVVAH |
| | TSVFRGKLICPNCGYALTLNSQKRKRKNDTIVYKTYYCNNCKITKGMKPHHITETETLRVFKDHLS |
| | KIDLKQYETQEKEKQSHVTIDLSKVMEQRKRYHKLYASGMMQENELFELIKETDEMIEEYEKQRK |
| | QVDVKEFDIGKIKEIKNVLLKSWDIFTLEDKADFIQMSIKAINIEYTKLKRGKASNSMKIKDIEFY |
| 192 | MTILDTPPTFRGLPPADDDAEKWLAYLRVSTWREDKISLDLQRTAIQAWERRGPRRVVEYVEDPD |
| | VTGRNFKRKIMGCIRRVEAGEIRGIVVWKFSRFGRNDMGIAVNLARVEKAGGDLVSATEDVDART |
| | AVGRFNRRILFDLATFESDRAGEQWKETHQWRRAHGLPATGGRRLGYIWHPRRIPHPTDPGQWTI |
| | QREWYEVEERARDHIEDLYARKIGDGYPVPDGYGSLAAWLNGLGYRTGDGNPWRADSLRRYMLS |
| | GFAAGLLRVHHPDCRCDYTANGGRCTRWIHIDGAHEAIITPETWERYEAHVAERRRMTPRARNPT |
| | YPLTGLIRCGGCREGAAATSARRASGRVLGYAYMCGQSRNGLCENPVWVQRYIVEDEVRGWLAR |
| | EVAADVDAAPATPEPVERDNRRAREERERARLEGEHTRLTNALTNLAVDRAMNPESYPEGVFEAA |
| | RERIVKQKQAVAEALEALAAVEATPERAALMPLAVGLLEEWETFEAPETNGILRSLVRRVALTRGA |
| | KGKKGVEGSGETRIEVHPVWEPDPWADDAPQ |
| 193 | MNYERRYLRSCQVSTLEQKEHGYSIEEQERKLKSFCEINDWSVSDVFIDAGFSGAKRDRPELQRMM |
| | NDIKRFDLVLVYKLDRLTRNVRDLLDLLEVFEQNNVAFRSATEVYDTSTAMGRLFVTLVGAMAE |
| | WERETIRERVMMGKRAAIKQGMILTPPPFYYDRVDNTYIPNNYKKVVLWAYDEVLKGVSSKGIAR |
| | KLNDSDIPPPNGKRWEDRTITRALRSPITRGHYTWGDVFIENSHEPIITEEMYQQIKERLEERINTKIV |
| | SHVSVFRGKFICPRCGGTLTLNTVTRKRKKGYVTYKTYYCNTCKAKKESFGFSENEALRVFRDYLS |
| | ELDLDKYKVKTKQNDDVVTIDIDKIMEQRKRYHKLYAKGLMQEEELFELIKETDETIAEYEKQKEL |
| | VPRKSLDIDKIKKFKNALLESWKIFSLEDKADFIKMAIKSIDIEYVKLKNRHSIKINDIEFY |
| 194 | MLKRAALYIRVSTDQQAKHGDSLDAQIATLKDYVSTQDNLTIIDTYIDDGISGQKLYRDEFQRLLED |
| | IKKNRIDIILFTKLDRWFRNLRHYLNIQEILDNSGVTWLAVSQPFFNTDTAYGRSFVNQSMSFAELE |
| | AQMASERIKAVFENKIRKGEVVTGSVPFGYKICDKKLIPNENAPIAKDIFKHYSIHNSIRLTVEYLFN |
| | EYDITRSSRTIKHMLRNRKYIGEVSGNKNYCPPIVDKETFEKVQNLLDKNISSIAKRTYIFSGLVVCS |
| | CCGKKMTGRYRKRKYIKKDGTVMYYTKKVYRCNGNTYKRNKCPNKINIPEEILEEYLLNNIKADA |
| | ENIALAKQKKIAVSAPEKNNNSKILKKIERLKKAYLNEVISLDEYKKDRKELEQMIVQVKPKETIVFK |
| | SNWFKKNIESTYRDFDEEEKRFVWRSVLKNLIVDPHGKITINFLTKN |
| 195 | MKTIHKLARPQLPEPPKLKVAAYARVSTSSNEQLASLQTQITHYENHIQNNDQWEYVGVYYDEGIS |
| | GTKVEKRDGLHRLIKDAELGKIDLILTKSISRFSRNTVDCLNLVRKLTDIGVTIFFEKENINTGDMESE |
| | LLLSILSSLAESESYSHSENMKWANRKRMAKGIFKTVPPYGYQRKGADFYLIPDEAKVIEQIFKWAL |
| | EGVSAYQVAKRLNEKNIFTRKGSKWQASGINNILHNIVYTGTMLHQRYFNDDQFRKKKNNGELPM |
| | YRIDNNHPPIISWEDYERVQELITLRANAKGTSKGSQKYSQRYAFTKRIICDKCGCNYKRVHTAGK |
| | GNTKVVKWSCTGHLKNKDGCDALPITDESLKTAYLTMLNKLILGHTIVLEPLINTPVEGKASKQEL |
| | EKLSIEITKIDEKLEVLASLNASGVVSTKTALEEQGRLQMELNKLQEKQHKIMESVNGTSTQRIQLE |
| | QLHQFTKRSEMLTEWDEDLFLRFAERIVVYSRQEVSFELKCGLLLKERLEA |
| 196 | MNVAIYCRVSTLEQKEHGYSIEEQERKLKSFCEINDWTVADVFVDAGFSGAKRDRPELQRLMNGIK |
| | RFDLVLVYKLDRLTRNVRDLLDLLEIFEQNNVAFRSATEVYDTSTAMGRLFVTLVGAMAEWERET |
| | IRERTQMGKLAALKKGIMLTTPPFYYDRVDNKFVPNKYKEVVLFAYEEALKGKSSKSIARKLNNSD |
| | IPPPNNRKWEDRSITRALRSPFTRGHFEWGGVYLENNHEPIITEEMYEKVKDRLEERTNTKKIKHVSI |
| | FRSKLVCPVCDSKLTMNTHKVTLKDRVYYNKHYYCNNCKETPNLKPVYIRAEEVERVFYEYLQH |
| | QDLTQYEVVEDTEEKEVAIDINKVMQQRKRYHKLYANGLMNEDELAELIEETDAAIEEYKKQNEN |
| | KEVKQYSDEDITEYKSLLLEMWNISSDEEKAEFIQMAIKNIFIEYVLGKNDNKKKRRSLKIKDIEFY |
| 197 | MSKARVYSYLRFSDPKQAAGSSADRQIEYARRWAAERNLELDDTLSLRDEGLSAYHQRHVKQGA |
| | LGVFLSAAEGGRIAPGSVLIVEGLDRLSRAEPIQAQAQLAQIVNAGITVVTASDGKEYNRERLRSQP |
| | MDLVYSLLVMIRAHEESDTKSKRVKAALRRQCQQWIDGKWRGIIRSGRDPHWVEIRDGQFALVPE |
| | RVAAVREALALFSRGHGKTKILRTLTERGLSMSNAGNHGTFIYRLVRNPMLMGTRVFEIDKEEFRL |
| | EGYYPALLSPEEFAVLQHLADERKGTRVKGEIPGLLTGLGITHCGYCGAAMVAQNYMGRARKAD |
| | GTPQDGHRRLHCVSDSQNSGCVVAGSVSIVPIERAIMTFCADQMNLTKLVEGDDGSAAVAGRLAL |
| | ARQKARGLQAQLERLTTALLADDGNAPPATFLRRARELEEELSSERRAIESLEREVLASANTTAPAA |
| | ADVWAKLTHGVLALDYESRVRARQLVADTFSRIVIFHAGFRPGEGTEKRIGIQLVAKHGNVRMLD |
| | VDRKSGDWRAAEDFDLRALT |
| 198 | MKTAIYLRKSRADLEAEARGEGETLAKHRTTLLKIAKEMNLNVLSVREEIVSGESLVKRPEMLALL |
| | EEIEDNKYDVVLCMDMDRLGRGGMKEQGIILETFKRSNTKIMTPRKTYDLNDEWDEEYSEFEAFM |
| | ARKELKIITRRMQRGRVASVEAGNYLGTHAPFGYDIHRLNKRERTLTINSEEASVVRMIFDWYANE |
| | DMGANAIRSKLNDLGYKSKLGNEWNPYSILDILKNNVYIGKVTWQKRKEVKQPDAVKRSCARQD |
| | KSDWIIADGKHEPIIPESLFEQVQEKLNSRYHVPYNTNGIKNPLAGIIKCSKCGYSMVQRYPKNRKE |
| | TMDCKHRGCENKSSYTELIEKRLLEALKEWYINYKADFEKHKQDDKLKETQVIQINEAALRKLEKE |
| | LVDVQKQKSNLHDLLERGVYTVDMFLERSNVVSDRITEITSTMENLKKEIKTEIKKEKVKKDTIPQV |
| | EHVLDLYFKTDDPKKKNNLLKSVLEKAVYKKEKWQRLDDFELVLYPKLPQDGDI |
| 199 | MRTALYIRVSTEDQAREGYSIQAQKNKLEAYCVSQGWDIAGFYVDDGYSAKDLERPEMKRMIKHI |
| | KQGLIDCVLVYRLDRLTRSVLDLYKLLELFEKHNCKFKSATEVYDTTTAMGRMFITIVAALAQWE |
| | RENLAERVRMGLQEKARQGKWVINKAPFGYDIDRESDTLVINEKEAAVVRKIFDLYISGKGMSKIA |
| | VELNKSQIHTKSGFGWSDSKIKYILKNPVYIGTMRYNYRVNQENYFEVKNAVPAIISEETFEKAQKI |
| | MNKRSKVHPKAATSEFIFSGIARCARCGGPLSGKHGYSKRKTKTHKLKTYYCYNRRYGLCDLPYM |
| | SERFIEQQFLKLIETIEIQDEILDDLQHNDEDSKERIKAIQNELKAIEKRRIKWQYAWANETISDEDFA |
| | QRMKEENEKEEELKKELEKIQPKQGEMMSIDKLKELAKDIRNNWEYMEPLEKKSLLQMIVKEMVI |
| | DKISLQPKPESVKIVDIKFY |
| 200 | MDNTSYIIKYVALYLRKSRGEEDIDLEKHRFILREMCVKHGWKYVEYVEIANSETIEYRPKFKSLLS |
| | DVEEGIYDAVLVVDYQRLGRGELEDQGKIKRIFRDSETYIVTPEKIYNLVDDTDDLLVDVRGLLAR |
| | QEYKTTTKNLQRGKKIGARLGKWTNGPAPFPYVYTAAIKGLEVVPERNVIYQEMKSRVLGGESLE |
| | AIGWDFNRRGIPGPGPKKGLWHSNTIGRILISEVHLGKIISNKTKGSGHKKKKTQPLVINPREEWVV |
| | VENCHAAVKTEEEHMKLLAMLEKNQVVPNRAKAGTYALSGLVFCGKCKKMMRYNVRSDGYTT |
| | NSIKACNKYDHFGNYCTNSGVKVNILTDFIDREIIDYEQRIIDSDNYINTDVIEKLERIIREKEAQLTKL |
| | NRALSKIKEMYEMEEYTREEYEERKAKRQQEISALESELAVHRYEINYDSREKNKERMKLINSFKDI |
| | WSSESATEHDKNMIAKMIISRIEYIHDKGTNNLNISIQFN |
| 201 | MKVAIYTRVSTHEQSLHGFSIEEQERKLKQFCEFNDWKVYKIYTDAGYSGAKRDRPALNQLIQDV |
| | DKLDLVLVYKLDRLTRSVRDLLDILEILEKNDVSFRSATEVYDTSTAMGRLFVTLVGAMAEWERTT |
| | IQERTFMGRRAAAQKGLIKTTPPFFYDRVDNKFIPNEYSKVLRFAVDEIKKGTSLREITIKLNNSNYK |
| | PPIGNRWHRSVLRNALKSPVARGHYYFSDVFVENTHEPIISDEEYEEIRERISERTNSVVVRHTSVFR |
| | GKLVCPVCGNRCTLNTNKHVTQKRGTWYSKHYYCDRCKCDKSVENFNFSEEEVLKQFYTYISNFD |
| | LTNYEVEMAEEEEPEIEIDIDKINEERKRYHILFAKGLMREDELTPLIKDLDDMVAAYNKQIKENKIK |
| | VYDYEQIKNFKYSLLEGWERMDLELKAEFIKRAIKSIKIEYIKGVRGKRPNSINILDVDFY |
| 202 | MATKARVYSYLRFSDPKQAAGSSADRQLEYAKRWAAEHGMALDAALSMQDEGLSAYHQRHVTK |
| | GALGVFLAAIDEGRIPAGSVLIVEGLDRLSRAEPIQAQAQLAQIINAGITVVTASDGREYNRAGLKA |
| | QPMDLVYSLLVMIRAHEESDTKSKRVRAAIHRQCKGWQDGTWRGVIRNGKDPSWTRLDPETKAF |
| | QLVPERAEAVKLAIRMFRDGHGAVRIMRTLAEEGLQLTNGGNPAGQLYRILRNRALIGEKVLEIDG |
| | EEYRLAGYYPSLLSAEQFADLQQATEQRAKQKGTGEIPGLITGLRISYCGYCGSAMVAQNLMNRG |
| | RREDGGPQHGHRRLICVGNSQGMGCAVAGSCSVVPIEHAIMSYCADQMNLARLFEGGDRSEALAG |
| | KLAIARARVADTTAKVERITDAMLADDAGDAPAAFMRRARELETSLVEQQAEVDALEHELAAVA |
| | SSPTPAVAKAWADLQEGVKALDYDARTKARQLVADTFERISIYHRGTEPEQTRSWKGTIDLVLVA |
| | KRGSARILHVDRQTGEWRGGEEVRDLPDDPIQ |
| 203 | MNKVAIYVRVSTTMQAEEGYSIDEQIDKLKSYCKIKDWTVYDIYKDGGFSGGNIKRPAMERLISDA |
| | KRKKFDTVLVYKLDRLSRSQKDTLFLIEEVFDKNDISFLSLNESFDTSTAFGKAMIGILSVFAQLERE |
| | QIKERMLLGKIGRAKTGKSMMFSKVSFGYTYDKLKDELVVNQAESIIVRKIFDAYLGGLSLNKLRD |
| | YLNNNGIYRGDKPWNYQGLRRILSNPVYIGMIRYREEIYPGNHKAIIDIDDYNKTQEEIKKRQIKALE |
| | FSNNPRPFRSKYMLSGIAKCGYCGTPLQIILGSKRKDGTRNMRYQCINRFPRNTKGVTIYNDGKKCE |
| | SGFYEKADIEEFVINEIRSLQINYNKLDAMFDRHPTVNSDDIKKQIITLDNKLKRLNDLYINNMIELD |
| | DLKKQTQSLRKQKTILEDELLNNPAITQEKNKKHFKEMLATKDITKLDYETQKNIVNNLINKVFVK |
| | SGYIKIEWKIPFKKA |
| 204 | MRKVAIYSRVSTINQAEEGYSIQGQIEALTKYCEAMEWKIYKNYSDAGFSGGKLERPAITELIEDGK |
| | NNKFDTILVYKLDRLSRNVKDTLYLVKDVFTANNIHFVSLKENIDTSSAMGNLFLTLLSAIAEFERE |
| | QIKERMQFGVMNRAKSGKTTAWKTPPYGYRYNKDEKTLSVNELEAANVRQMFNMIISGCSIMSIT |
| | NYARDNFVGNTWTYVKVKRILENETYKGLVKYREQTFSGDHQAIIDEKTYNKAQIALAHRTDTKT |
| | NTRPFQGKYMLSHIAKCGYCGAPLKVCTGRAKNDGTRRQTYVCVNKTESLARRSVNNYNNQKIC |
| | NTGRYEKKHIEKYVIDVLYKLQHDKEYLKKIKKDDNIIDITPLKKEIEIIDKKINRLNDLYINDLIDLP |
| | KLKKDIEELNHLKDDYNKAIKLNYLDKKNEDSLGMLMDNIDIRKSSYDVQSRIVKQLIDRVEVTMD |
| | NIDIIFKF |
| 205 | MTDPTLTRSKKPAYIYARFSSLEQAKGFSLERQLTTARSYIERKGWQLAEELADEGRSAFKGSNRD |
| | EGAALFEFESRARSGHFKNGAVLVVESIDRLSRQGPKAAAQLIWSLNENGVDVASYHDDQVYRAG |
| | SGDMLEIFGLIIKASLAHEESDKKSKRAKASWEKKYGDIEAGSKKAITKQVPAWLTVTADNDIIENP |
| | ARVKVVREIFEWYVEGIGLHTIMKRLNERGEPAFSGRETSKGWSKSAINHVLSNRAVLGEFATQQG |
| | KHIPVVYYPQVVSRDLFNRAEAMRATKTRTGGSSKYQGNNLFAGIAKCEVCDGPMGFVRDGGISR |
| | YTTASGEQRVYKSKGHNYLICDAARRGFGCDNKVHAPYATLEAATLQQLLWATIDDEEAQADPK |
| | ADALRSKLDAVLHSIDLKNQQISNIIDSMAEAPSKAMAARVAALEAETDALGAECDELQKALAVQ |
| | TSAPSLRDDIAQLRDLTELMNSEDEDVRRAARLRTNASLKRVIDHMTIDRAANVTVMSMDVGVW |
| | QFDKLGNRIGGQAL |
| 206 | MKAAIYIRVSTQEQVENYSIQAQTEKLTALCRSKDWDVYDIFIDGGYSGSNMNRPALNEMLSKLHE |
| | IDAVVVYRLDRLSRSQRDTITLIEEYFLKNNVEFVSLSETLDTSSPFGRAMIGILSVFAQLERETIRDR |
| | MVMGKIKRIEAGLPLTTAKGRTFGYDVIDTKLYINEEEAKQLRLIYDIFEEEQSITFLQKRLKKLGFK |
| | VRTYNRYNNWLTNDLYCGYVSYKDKVHVKGIHEPIISEEQFYRVQEIFARMGKNPNMNRDSASLL |
| | NNLVVCSKCGLGFVHRRKDTMSRGKKYHYRYYSCKTYKHTHELEKCGNKIWRADKLEELIIDRV |
| | NNYSFASRNVDKEDELDSLNEKLKTEHTKKKRLFDLYISGSYEVSELDAMMADIDAQINYYEAQIE |
| | ANEELKKNKKIQENLADLATVDFDSLEFREKQLYLKSLINKIYIDGEQVTIEWL |
| 207 | MTVGIYIRVSTDEQVKEGFSISAQKEKLKAYCTAQGWEDFKFYVDEGKSAKDMHRPLLQEMITHIK |
| | KGLIDTVLVYKLDRLTRSVVDLHNLLSIFDEYNCAFKSATEVYDTSSAMGRFFITIISSVAQFERENT |
| | SERVSFGMAEKVRQGEYIPLAPFGYVKGPDGKLIVNEAEKEIFLHVVNMVSTGYSLRQTCEYLTNI |
| | GLKTRRSNDMWKVSTLIWMLKNPAVYGAIKWNNEIYENKHEPLIDKATFDKLANILSIRSKSTTSR |
| | RGHVHHVFKGRLICPQCGKRLSGLRTKYVNKNKETFYNNNYRCATCKEHRRPAIQISEQKIEKAFI |
| | DYISNYTLNKADISSKKLDNNLRKQEMIQKEIVSLQRKREKFQKAWAADLMSDDEFSKLMIDTKM |
| | EIDVAEDRKKEYDVSLFVSPEDIAKRNNILRELKINWTSLSPTEKTDFISMFIEGIEYVKNDENKAVIT |
| | KIRFL |
| 208 | MTVGIYIRVSTEEQANEGYSISAQRERLKAFCLAQNWHDYKFYVDEGISGRDTKRPQLKKMMEDI |
| | KAGHINVLLVYRLDRLTRSVRDLHRILDELEKYSCTFRSATEFYDTSTAMGKMFITIIAAIAEWESA |
| | NLGERVTMGQVEKARQGEWAAQPPYGFFKDDKHKLQIHKEEIKAVKLMVKKIREGMSFRQLAFY |
| | MDSTQYKPKRGYKWHVRTLLSLMHNPALYGAMYWKEQIYENTHQGIMTKEEFDQLQKIISSRQN |
| | YKSRNVSSHFVFQTKLICPDCGSRCTSERYTWKRKTDNAVEVRNSYRCQVCALNNPKSTPFSVREV |
| | KVDEALIEYMINFTVAPSEVVELNENDQLLDIKNNLRKIENQREKYQRAWANDLITDDEFKVRMDE |
| | SRLQFDSLQNDLKNIEGEKYDVVDIERYIEITKTFNDNYLNLTQEERRTFIQTFIESVKVEIVEHTKGK |
| | GYRNQKIRIADVSFY |
| 209 | MTVGIYIRVSTEEQAREGFSISAQREKLKAYCVSQDWTDYKFYVDEGKSAKDTNRPYLKLMLDHI |
| | QQGLIDVVLVYRLDRLTRSVKDLYKLLDLFDKNNCIFRSATEVYDTGSATGRLFITLVAAMAQWE |
| | RENLGERVSMGQVEKARQGEFSAPAPFGFRKQGETLIKDEKQGPILLDIIEKVKKGWSIRQVAKFLD |
| | ESEHMPIRGYKWHIGTILSILHNPALYGAFRWKDEIYEDSHEGYITKEEFEELQEILYSRQNFKKREV |
| | KSNFIFQTKLVCPQCGNRLGCERSVYFRKKDQKNVESHHYRCQSCALNYKPAVGVSEKKIEKALLT |
| | YMKNVTFDLKPIVKEEKDDSLEIQNQIKKIERKREKFQKAWASDLMTDEEFAARMSETKNAYEEL |
| | KKQLSEIQPNEDLTVDIKKAKKLVNEFKLNWSYLNHAEKREYVQSFIEKIEFEKKGLTPRIRNVSFY |
| 210 | MKVAIYTRVSTLEQKEKGHSIEEQERKLRAYSDINDWKIHKVYTDAGYSGAKKDRPALQEMLNEI |
| | DNFDLVLVYKLDRLTRSVKDLLEILELFENKNVLFRSATEVYDTTSAMGRLFVTLVGAMAEWERT |
| | TIQERTAMGRRASARKGLAKTVPPFYYDRVNDKFVPNEYKKVLRFAVEEAKKGTSLREITIKLNNS |
| | KYKAPLGKNWHRSVIGNALTSPVARGHLVFGDIFVENTHEAIISEEEYEEIKLRISEKTNSTIVKHNAI |
| | FRSKLLCPNCNQKLTLNTVKHTPKNKEVWYSKLYFCSNCKNTKNKNACNIDEGEVLKQFYSYLKQ |
| | FDLTSYKIENQPKEIEDVGIDIEKLRKERARCQTLFIEGMMDKDEAFPIISRIDKEIHEYEKRKDNDKG |
| | KTFNYEKIKNFKYSLLNGWELMEDELKTEFIKMAIKNIHFEYVKGIKGKRQNSLKITGIEFY |
| 211 | MKAAIYIRVSTQEQVENYSIQAQTEKLTALCRSKDWDVYDIFIDGGYSGSNMNRPALNEMLSKLHE |
| | IDAVVVYRLDRLSRSQRDTITLIEEYFLKNNVEFVSLSETLDTSSPFGRAMIGILSVFAQLERETIRDR |
| | MVMGKIKRIEAGLPLTTAKGRTFGYDVIDTKLYINEEEAKQLQLIYDIFEEEQSITFLQKRLKKLGFK |
| | VRTYNRYNNWLTNDLYCGYVSYKDKVHVKGIHEPIISEEQFYRVQEIFARMGKNPNMNRDSASLL |
| | NNLVVCGKCGLGFVHRRKDTVSRGKKYHYRYYSCKTYKHTHELEKCGNKIWRADKLEELIIDRV |
| | NNYSFASRNVDKEDELDSLSEKLKIEHVKKKRLFDLYISGSYEVSELDAMMADIDAQINYYEAQIE |
| | ANEELKKNKQIQENLADLATVDFDSLEFREKQLYLKSLINKIYIDGEQVTIEWL |
| 212 | MKKAIAYMRFSSPGQMSGDSLNRQRRLIAEWLKVNSDYYLDTITYEDLGLSAFKGKHAQSGAFSE |
| | FLDAIEHGYILPGTTLLVESLDRLSREKVGEAIERLKLILNHGIDVITLCDNTVYNIDSLNEPYSLIKAI |
| | LIAQRANEESEIKSSRVKLSWKKKRQDALESGTIMTASCPRWLSLDDKRTAFVPDPDRVKTIELIFK |
| | LRMERRSLNAIAKYLNDHAVKNFSGKESAWGPSVIEKLLANKALIGICVPSYRARGKGISEIAGYYP |
| | RVISDDLFYAVQEIRLAPFGISNSSKNPMLINLLRTVMKCEACGNTMIVHAVSGSLHGYYVCPMRR |
| | LHRCDRPSIKRDLVDYNIINELLFNCSKIQPVENKKDANETLELKIIELQMKINNLIVALSVAPEVTAI |
| | AEKIRLLDKELRRALVSLKTLKSKAVSSLGDFHAIDLTSKNGRELCRTLAYKTFEKIIINTDNKTCDI |
| | YFMNGIVFKHYPLMKTISAQQAISTLKYMVDGEVYF |
| 213 | MKKITKIDELPQGQLPNTKLRVAAYARVSTDSDEQLESLKAQREHYERYIKSNPEWVFAGLYYDEG |
| | ISGTKMEKRTELLRMIRDCKQGRIDFIITKSISRFARNTVDCLELVRKLIDIGVYIYFEKENLNTGDME |
| | SELMLSILSGFAAEESASISQNSKWSIQKRFQNGSYIGTPPYGYTNIDGEMVIVPEEAEIIKRIFSECLS |
| | GKGGGTIARGLNKDKIPARRGNHWSAGTVIDMLRNEKYMGDVLLQKTYTDSNYNRHPNTGEKDQ |
| | YYYKDNHEPIISREDFAKAQDLIDERAKMKCKGVKKNVYLNRYALSGKIVCGECGRNFRRKTNYS |
| | AGRSYIAWSCIGHIEDKESCSMLFLRDGEIKATLTTMMNKLAFSHKLILEPLFKSISQIDEESDRERM |
| | DAIDKRMEQLMEERNTLITLMAKGFLEPALFNQERNVLDSEIKNLTTEKTNLVTNSTSGVLRANDIK |
| | DLIDYVSADNFNGEYTEELFEEFVENIIVNSRDELTFNLKCGLSLKEKVVR |
| 214 | MVIPARKRVGSTAAKEKIKKLRVAAYCRVSTETEEQNSSYEVQVAHYTEFIKKNTEWEFAGIFADD |
| | GISGTNTKKREEFNRMIAECMDGNIDMVITKSISRFARNTLDCLQYIRQLKDKNISVYFEKENINTM |
| | DAKGEVLLTIMASLAQQESQSLSQNVKLGLQYRYQQGKVQVNHKRFMGYSKDEDGNLIIVPEEAE |
| | IIKRIYREYLEGQSLVGIGQGLEKDGILTAAGKPRWRPESVKKILQNEKYIGDALLQKTVTVDFLTK |
| | KRVKNEGHVPQYYVENSHEAIIPKDLFLQVQEEIHRRRNIYTGADKNKRIYSSKYALSAITFCGDCG |
| | DIYRRTYWNIHGRKEFVWRCVTRIEQGPEVCKNRTVKEDELYGAVMTATNRLLAGGDNMIRTLEE |
| | NIHAVIGDTTEYQISELNSLLEENQKELISLANKGKDYESLADEIDELREKRQTLLIEDASLSGENERI |
| | NELIEFVRDNKYCTLRYDDTLVRKIIQNVTVYEDHFVIGFKSGIEIEVE |
| 215 | MKAAIYIRVSTQEQIENYSIQAQTEKLTALCRSKDWDVYDIFIDGGYSGSNMNRPALNEMLSKLHEI |
| | DAVVVYRLDRLSRSQRDTITLIEEYFLKNNVEFVSLSETLDTSSPFGRAMIGILSVFAQLERETIRDR |
| | MVMGKIKRIEAGLPLTTAKGRTFGYDVIDTKLYINEEEAKQLQMIYDIFEEEKSITTLQKRLKKLGF |
| | KVKSYSSYNNWLTNDLYCGYVSYADKVHTKGVHEPIISEEQFYRVQEIFSRMGKNPNMNRDSASL |
| | LNNLIVCGKCGLGFVHRRKDTVSRGKKYHYRYYSCKTYKHTHELEKCGNKIWRADKLEELIIDRV |
| | NNYSFASRNVDKEDELDSLNEKLKTEHTKKKRLFDLYISGSYEVSELDGMMADIDARINYYEAQIE |
| | ANEELKKNKKIQENLADLATVDFDSLEFREKQLYLKSLINKIYIDGEQVTIEWL |
| 216 | MKVAIYTRVSSAEQANEGYSIHEQKKKLISYCEIHDWNEYKVFTDAGISGGSMKRPALQNLMKQL |
| | SYFDLVLVYKLDRLTRNVRDLLDMLEEFEQYNVSFKSATEVFDTTSAIGKLFITMVGAMAEWERET |
| | IRERSLFGSRAAVREGNYIREAPFCYDNIEGKLHPNEHAKVIDLIVSMFKKGISANEIARRLNSSKVH |
| | VPNKKSWNRNSLIRLMRSPVLRGHTKYGDMLIENTHEPVLSEHDYNAINDAISSKTHKSKVKHHAI |
| | FRGALVCPQCNRRLHLYAGTVKDRKGYKYDVRRYKCETCSKNKDVKNVSFNESEVENKFINLLKS |
| | YELNKFHIRKVEPVKKIEYDIDKINKQKINYTRSWSLGYIEDDEYFELMEEINATKKMIEEQTTENK |
| | QSVSKEQIQSINNFILKGWEELTIKDKEELILSTVDKIEFNFIPKDKKHKTNTLDINSIHFKF |
| 217 | MKVAIYTRVSSYEQATEGYSIHEQERKLKAFCEVQNWHNFKVFTDAGVSGGSMNRPALKRIMDNL |
| | EYYDLVLVYKLDRLTRNVKDLLEMLEKFEKYNVAFKSATEVFDTTTAIGKLFITMVGAMAEWERA |
| | TIRERALFGSRAAVREGNYIREAPFCYDNVDGKLVPNKHKWVIDYLVEQFKHGVSGNEIARQMNL |
| | KKVNVPKVKKWNRTSIIRLMKNPVLRGHTKYGDMYIENTHEPVLSESDYKRIIDVIENKTHRSKVK |
| | HHAIFRGVLTCPQCHNKLHLYAGKITDKKGYSYEVRRYKCDTCSKDKNVQTISFNESEVEDKFIEL |
| | LKTYDMNKFKVDIVEESTPKLDYDIDKIMKQREKLTRSWSLGYIEDDEYFSLMDETKEILDEVERG |
| | GTEVESTQTVTNEQLNMIDDILIKGWSKLNVEQKEELILSTVKEIAFDFVPRKDNESGKVNTLNIREI |
| | TFKF |
| 218 | MKAAIYSRKSKFTGKGESIENQIEMCKKYASDNEYDEIFIYEDEGFSGGNINRPEFKQMMKDAKSH |
| | KFDVIICYRLDRISRNVSDFSTLIDKLKLLNIGFISIKEQFDTTSPMGTAMMFISSVFAQLERETIAERI |
| | KDNMYELAKTGRWLGGTPPFGFISEQSLYSDTNGKQKKMFQLAPVGSECELIKYMYEKYLALGSL |
| | GKLQKHLSSKEIKTRNNATWDIKALQLILRNPVYVKSDEVVLSYLESKGAKVFGEVNGNGILSYNK |
| | KDSKDKYKDISEWILSVAKHNGLIDSSLWLLVQKKLDKNKSLAPRLVSNDSSGLLSRVLYCKKCG |
| | GKMIQKKGHTSVKTKEPFRYYVCLNKMNFKSCDSKNIRADILEKHVADKIIEETSDTGSLIKAIDDY |
| | KNKLQLDSGKSNNLNFIKKQILLKQTQINNLMENISKNPKLFDLFNSKIEELNSELKSLKFKKFEAES |
| | VKENTSNALKEIDASTQMLLNFKRLWMYADSSTKKLLIENIVDSVCYDADNKTADVKLICCKKKG |
| | AL |
| 219 | MKAAIYIRVSTQEQIENYSIQAQTEKLTALCRSKDWDVYDIFIDGGYSGSNMNRPALNEMLSKLHEI |
| | DAVVVYRLDRLSRSQRDTITLIEEYFLKNNVEFVSLSETLDTSSPFGRAMIGILSVFAQLERETIRDR |
| | MVMGKIKRIEAGLPLTTAKGRTFGYDVIDTKLYINEEEAKQLQMIYDIFEEEKSITTLQKRLKKLGF |
| | KVKSYSSYNNWLTNDLYCGYVSYADKVHTKGVHEPIISEEQFYRVQEIFSRMGKNPNMNRDSASL |
| | LNNLVVCGKCGLGFVHRRKDTVSRGKKYHYRYYSCKTYKHTHELEKCGNKIWRADKLEELIIDRV |
| | NNYSFASRNVDKEDELDNLNEKLKTEHTKKKRLFDLYISGSYEVSELDAMMADIDAQINYYEAQIE |
| | ANEELKKNKKIQENLADLATVDFDSLEFREKQLYLKSLINKIYIDGEQVTIEWL |
| 220 | MKAAIYIRVSTQEQIENYSIQAQTEKLTALCRSKDWDVYDIFIDGGYSGSNMKRPALNEMLSKLHEI |
| | DAVVVYRLDRLSRSQRDTITLIEEYFLKNNVEFVSLSETLDTSSPFGRAMIGILSVFAQLERETIRDR |
| | MVMGKIKRIEAGLPLTTAKGRTFGYDVIDTKLYINEEEAKQLQMIYDIFEEEKSITTLQKRLKKLGF |
| | KVKSYSSYNNWLTNDLYCGYVSYADKVHTKGVHEPIISEEQFYRVQEIFSRMGKNPNMNRDSASL |
| | LNNLVVCGKCGLGYVHRRKDTVSRGKKYHYRYYSCKTYKHTHELEKCGNKIWRADKLEELIIDR |
| | VNNYSFASRNVDKEDELDSLNEKLKTEHAKKKRLFDLYISGSYEVSELDGMMADIDAQINYYEAQI |
| | EANEELKKNKKIQENLADLATVDFDSLEFREKQLYLKSLINKIYIDGEQVTIEWL |
| 221 | MNYERRYLRSCQVSTLEQKEHGYSIEEQERKLKSFCEINDWSVSDVFIDAGFSGAKRERPELQRMM |
| | NDIKRFDLVLVYKLDRLTRNVRDLLDLLEIFEQNNVAFRSATEVYDTSTAMGRLFVTLVGAMAEW |
| | ERETIRERVMMGKRAAIKQGMILTPPPFYYDRVDNTYIPNDYKKVVLWAYDEVMKGNSSKAIARK |
| | LNDSDIPPPNGKRWEDRTITRALRSPITRGHYTWGDVFIENSHEPIITEEMYQQIKERLEERINTKIVS |
| | HVSVFRGKFICPRCGGTLTLNTVTRKRKKGYVTYKTYYCNTCKAKKQSFGFSENEALRVFRDYLS |
| | KLDLEKYEVKTKQKDDVVTIDIDKIMEQRKRYHKLYAKGLMKEEELFGLIKETDETIAEYEKQKEL |
| | VPRKSLDIDKIKKFKNALLESWEIFSLEDKADFIKMAIKSIDIDYVKLKNRHSIKINDIEFY |
| 222 | MENKIKCGIYARVSTDRQGDSIENQVGQGTEYIKRLGDEYDTENIEVFRDEAVSGYYTSVFDRAEM |
| | KRAIEYAREKKIQLLVFKEVSRVGRDKQENPAIIGMFEQYGVRVIAINDNYDSMNKDNITFDILSVL |
| | SEQESRKTSVRVSTARKQKAARGQWNGEPPYGYIVNPETKRLEIHEERGKIPPLVFDLYVNRGMGT |
| | FKVAEYLNKKGYVTKNGKLWSRETVNRLIRNQAYIGQVAYGTRRNVLKREYDERGAMTKKKVQI |
| | KINRQEWQIVEDAHPALVDKELFYKAQKILMSRTHERGGAKRAHHPLTGVLVCGSCGEGMVCQK |
| | RSFKDKEYRYYICKTYHKYGREACSQANINADDIERAVVEAVRNKISRLPADTLLITADREQDIKKL |
| | TSELKDNNSRRDKLMKDQLDIFEQRELFPDDLYRSKMIEIKNSIAHLEEEKEIIEKQIEGIKEKITESSS |
| | LQHIIEEFKELDIEDVGRLRVLIHETVGSITVKGDNLRIEYVYDFDS |
| 223 | MDRICIYLRKSRADEELEKTIGEGETLSKHRKALLKFAKEKKLNIVEIKEEIVSADSIFFRPKMIELLK |
| | EVETKRYIGVLVMDIQRLGRGDTEDQGIITRIFKESHTKIITPQKTYDLDDDLDEDYFEFESFMGRKE |
| | YKMIKKRMQGGRVRSVEDGNYIATNPPFGYDVHWINKSRTLKANSKESEIVKLIFKLYIKGNGAGT |
| | IAKHLNDLGYKTKFGNNFSNSSVIFILKNPVYIGKITWKKKDIKKSKDPNKVKDTRTRDKSEWIIAD |
| | GKHKAIIDSNIWNKAQEILSNKYHIPYKLANPPANPLAGLVICSKCNGKMVMRKYGKKLPHLICTN |
| | TKCNNKSARFDYIEKAILEGLEEYLKNYKVNVKGNGKKANLKPYEQQLNALSKELIVLNEQKLKLF |
| | DFLEREVYTEEIFLERSKNLDERINTSTLAINKIKKILDDEKKKNNKNDIVKFEKILEGYKETKDIQKK |
| | NELMKSLIFKIEYKKEQHQRNDDFDIRLFPKLLR |
| 224 | MKAAIYIRVSTQEQIENYSIQAQTEKLTALCRSKDWDVYDIFIDGGYSGSNMNRPALNEMLSKLHEI |
| | DAVVVYRLDRLSRSQRDTITLIEEYFLKNNVEFVSLSETLDTSSPFGRAMIGILSVFAQLERETIRDR |
| | MVMGKIKRIEAGLPLTTAKGRTFGYDVIDTKLYINEEEAKQLQMIYDIFEEEKSITTLQKRLKKLGF |
| | KVKSYSSYNNWLTNDLYCGYVSYADKVHTKGVHEPIISEEQFYRVQEIFSRMGKNPNMNRDSASL |
| | LNNLVVCGKCGLGYVHRRKDTVSRGKKYHYRYYSCKTYKHTHELEKCGNKIWRADKLEELIIDR |
| | VNNYSFASRNVDKEDELDYLNEKLKIEHAKKKRLFDLYINGSYEVSELDSMMNDIDAQINYYESQI |
| | EANEELKKNKKIQENLADLATVDFDSLEFREKQLYLKSLINKIYIDDEQVTIEWL |
| 225 | MKAAIYIRVSTQEQIENYSIQAQTEKLTALCRSKDWDVYDIFIDGGYSGSNMKRPALNEMLSKLHEI |
| | DAVVVYRLDRLSRSQRDTITLIEEYFLKNNVEFVSLSETLDTSSPFGRAMIGILSVFAQLERETIRDR |
| | MVMGKIKRIEAGLPLTTAKGRTFGYDVIDTKLYINEEEAKQLRLIYDIFEEEQSITFLQKRLKKLGFK |
| | VRTYNRYNNWLTNDLYCGYVSYKDKVHVKGIHEPIISEEQFYRVQEIFSRMGKNPNMNKESASLL |
| | NNLVVCSKCGLGFVHRRKDTVSRGKKYHYRYYSCKTYKHTHELEKCGNKIWRADKLEELIIDRVN |
| | NYSFASRNIDKEDELDSLNEKLKIEHAKKKRLFDLYINGSYEVSELDSMMNDIDAQINYYESQIEAN |
| | EELKKNKKIQENLADLATVDFNSLEFREKQLYLKSLINKIYIDDEQVTIEWL |
| 226 | MEDSSNKSVGIYVRVSTDEQAKEGFSISAQKEKLKAYCVSQGWANFKFYVDEGKSAKDTHRPSLE |
| | LLLRHIEQGIIDTVLVYRLDRLTRSVRDLYTLLDYFDKYNAVFRSATEVYDTGSATGRLFITLVAAM |
| | AQWERENLGERVKMGQNEKARQGQFSAPAPFGFIKEGKSLVKNHEQGEILLEIIDKVKKGYSTRQI |
| | ANYLDDSGLLPIRGYRWHPGTILTLLKNPILYGSFRWGDEIIEDTHEGYISKDEFDRIQEILKERSIVK |
| | KRDSYSVFIFQSKIVCAGCGNRLASERSKYFRKKDKQYVETNNYRCQTCAQNRKPSIMGSEKKFQK |
| | ALVKYMQNVTPKLEPKIPEEKKHDYEKVHQKILNLEKQRKKYQKAWSLDLMTDEEFEQLMYETK |
| | EALKSAQNELAAAHSSDSQNSQIDIERAKEIVKMFNENWSVLTNEEKRSIVQELIKHINFTKEDGEIII |
| | THIEFY |
| 227 | MSSVRRNQTPAITPKKRCAVYTRKSTDEGLDQEYNSLEAQRDAGLAFIASQRHEGWIAVDDGYDD |
| | GGYSGGNMERPGLRRLMIDIEAGKIDTVVVYKIDRLTRSLPDFAKLVDVFDRNGVSFVSVTQQFNT |
| | TTSMGRLTLNILLSFAQFEREVTGERIRDKIAASKAKGMWMGGVPPLGYDVVERKLVVNEREAVL |
| | VRDIFRRYAEHGSAARLVRELEIEGHTTKAWVTQSGRERLGRSIDQQYLFTLLRNRIYLGEICNHDT |
| | WYSAQHDPIISQELWDAAHAFIERRKQAPREHRAKHPALLAGLLFAPDGQRMLHSFVKKKNGRQY |
| | RYYVPYLHKRRNAGASLAPHTPDVGHLPAAEIEEAVLAQIHAALSSPQILIAVWRSCQQHPVGAAL |
| | DEAQVVVAMQRIGDVWSQLFPAEQQRITRLLIERVQLHGHGLDIVWREDGWIGFGADISTHPLIEES |
| | QERVEEVWA |
| 228 | MQAEEFSIPGADQPPTFRAAEYVRMSTEHQQYSTENQADKIREYAARRNIEIVRTYADEGKSGLRID |
| | GRRALQQLIKDVETGSADFQIILVYDVSRWGRFQDADESAYYEYICRRAGIQVAYCAEQFENDGSP |
| | VSTIVKGVKRAMAGEYSRELSAKVFAGQCRLIELGFRQGGPAGYGLRRVLVDQSGTLKGELARGE |
| | HKSLQTDRVILQPGPDDEVAVVNQIYRWFVADNMTELDIAERLNAQGTRTDLGRDWTRATIREVL |
| | SNEKYIGNNIYNRRSFKLKKHRVVNSPEMWIKKEGAFEGIVPPELFYTAQGILRARAHRYSDEELIE |
| | KLRNLYQRHGYLSGLIIDEAEGMPSSAAYAHRFGSLIRAYQTVGFTPDRDYQYLEANQFLRRLHPEI |
| | VGQTERMIAEVGGMVERDPATDLLTVNREFTVSLVLARCQLLDNGRRRWKVRFDTSLAPDITVAV |
| | RLDDSNQAALDYYLLPRLDFGQARIHLADHNGIEFECYRFDSLDYLYGMARRIRIRRAA |
| 229 | MKAAIYIRVSTQEQIENYSIQAQTEKLTALCRSKDWDVYDIFIDGGYSGSNMNRPALNEMLSKLHEI |
| | DAVVVYRLDRLSRSQRDTITLIEEYFLKNNVEFVSLSETLDTSSPFGRAMIGILSVFAQLERETIRDR |
| | MVMGKIKRIEAGLPLTTAKGRTFGYDVIDTKLYINEEEAKQLQMIYDIFEEEKSITSLQKRLKKLGF |
| | KVKSYSSYNNWLTNDLYCGYVSYADKVHTKGVHEPIISEEQFYRVQEIFSRMGKNPNMNRDSASL |
| | LNNLVVCSKCGLGFVHRRKDTVSRGKKYHYRYYSCKTYKHTHELEKCGNKIWRADKLEELIIDRV |
| | NNYSFASRNVDKEDELDNLNEKLKTEHTKKKRLFDLYISGSYEVSELDAMMADIDAQINYYEAQIE |
| | ANEELKKNKKIQENLADLATVDFNSLEFREKQLYLKSLINKIYIDGEQVTIEWL |
| 230 | MGKNGARVYSYLRFSDPRQATGSSADRQLAYASAWASKHGMELDATLTLRDEGLSAYHETHVK |
| | QGALGAFLRAVDEGRIPAGSVLIVEGLDRLSRAEPLLAQAQLGQIVNAGITVVTASDGREYNREGL |
| | KAEPMNLVYSLLVMIRAHEESDTKSKRVKAAVRRQCEGWVTGSYRGRIVSGKDPQWLAWDGDS |
| | WQFIPERVEAVRFALDAYRSGIGAARLVRLMHEKGMVLSDWGIAAQQVYRLVRLPALRGAKRISI |
| | DGEDFMLEDYYPRLLSDEEFSELETLVGQRYRRRGKDEIVGIVTGIGITRCGYCGTALVAQNLMQR |
| | VKSDGSLEDGHRRLHCVSYSKNGGCNGGSCSSVPIERAVLAYCSDQMNLQRLLEPSSAGEDLRPRL |
| | VEAKKGVAEIERQLERVTDALLADDTGAAPMAFVRKARELEEDLERRRSAVRALEQELVTKSAST |
| | PAAGASKWAELAERAKSMTDVEAREQARQLVMDTFETLVVYMRGVMPTPKGRHIDLMMRSRAG |
| | QTRWLRVDRRSGVWRESGDSSRRLEG |
| 231 | MKMKSVLYARVSTEDLEQNNSYIQQQLYQDDRFEIVKIFSDKASGSSVDGRESFLEMLKYVGISKE |
| | GNNYFVEHRTEIECIIVANVSRFSRSVVDARLIIDALHKNNVKVFFVDLNKFSDDADIFLQLNMYLM |
| | IEEQYLRDVSKKVKAGMQRKQSTGYILGSNKIWGYNYVTKDDGKGYLVPHETESLMVKNIFKEYI |
| | TGAGTRTLAKKYKLSSSTILGILKNTKYCGYMGYNLKSDNPTYVKSPFIEPLISTEAFEEVQRIIKGR |
| | CNSESGRGRRIKVRNLTGKIKCECGANYHYKQRETEWCCGREGVEGRTKGCGSPQFNTKLIIPYLE |
| | KNMDNIEKNLQFNLNREIKDINVGSFDRLNQRKEELIRQQDKLLDLYLDEDKLKNISKEMLERRSK |
| | LIKEEIEEVEEKLVILNDVNSHLNNLRRIKVEYKNEIKNIRRLIEEKNLDEIEKLISKIQLETIVNIINFR |
| | KELRIKEIQFSCFNELYNTNFIFAPEPKKVK |
| 232 | MNNKVAIYVRVSTHHQIDKDSLPLQRQDLINYTKYVLNINEYELFEDAGYSAKNTDRPNFQNMMT |
| | KIRNNEFSHLLVWKIDRISRNLLDFCDMYEELKKYNCTFVSKNEQFDTSSAMGEAMLKIILVFAELE |
| | RKLTGERVTAVMLDRASKGLWNGAPIPLGYVWDKVKKFPIIDRTEKSTIELIYNTYLKAKSTTEVR |
| | GLLNANGIKTKRGGSWTTKTVSDIIRNPFYKGTYRYNYKEPGRGKIKNKNEWIVIEDNHPGIIEKEL |
| | WKKCNEIMDVNAQRNNASGFRANGKVHVFAGILECGECYKNLYAKQDKPNIEGFRPSIYVCSGRY |
| | NHLGCSQKTISDNYVGTFIFNFISNILTVQRKIKKLDLEVLEKTLIKGKAFTNVVGIENIEVLQQLSYS |
| | ESTFKSKNIEDKENSFELEVIKKEKSKYERALERLEDLYLFDDESMSEKDYVLKKNKINEKLNDANE |
| | KLRKIDNYNDISELNLEKEASDFMLSKQLLNTECINYKNLVLNVGRDILKEFVNTIIDKIIVKDKKISS |
| | VKFKSGLVIKFVYKC |
| 233 | MNVAIYLRKSRADEEAEKQGEFETLSRHKSTLLKLAKEQNLDVIEIKEELVSGESIIHRPKMLELLKE |
| | VEENKYDAVLVMDLDRLGRGDMKDQGIILETFKESKTKIITPRKTYDLTDEFDEEYSEFEAFMARK |
| | ELKLISRRMQRGRIKSVEEGNFIGTSAPFGYDAVTTGRKERILVPNKDADVVRTIFDLYINEDMGCS |
| | KISKYLNNLGIKTATGANWYNSAITNIIKNKVYCGYIQWQKKDYKKSKNPNKIKTVKLRPKDEWIE |
| | AKGKHEPLISEITWKKAQNILKKNGHVSYGNQIKNPLAGIVICKNCARPLVYRPYADHDYIICYHPG |
| | CNKSSRFEFIEAAILKSLEDTVKKYQLKASDLDLDKNNKDSNIEFQKRVLKGLETELKELGKQKNK |
| | LYDLLERGIYDEDTFIERSNNISSRTEEIKDSINTVKNRLSTVKKDNSKIIEDIKTVLSLYHDSDSLGKN |
| | KLLKSVIDKAVYYKSKEQKLDSFELMVHLKLHEDQ |
| 234 | MSVIVTKKRCAVYTRVSTDERLDQSFNSLDAQREAGQAYIAAQRHEGWLPVDDDYDDGGYSGGN |
| | MERPALKRLLALIATDQIDVVVVYKIDRLTRSLVDFARLIEAFERHKVSFVSVTQQFNTTTSMGRL |
| | MLNILLSFAQFEREVTGERIRDKIAASKRKGMWMGGYPPLGYDLKDRKLFVNEREAPTVQRIFERF |
| | AALGSVTELCRELAQDGVKTKAWQTRDGRMRNGTVMDKQYLSKALRNPVYVGEIRHKNVVHAG |
| | QHTPIISRQLWDRVQAILAADADQRAGMTRTRGKCDALLRGLLFGPNGEKYYPTFTKKASGKRYR |
| | YYYPQSDKKYGFGSSALGMLPADQIEEVVVNLVIQALQSPESMQAVWDHVRQNHPEIDEPTTVLA |
| | MRQLGEVWKQLFPEEQVRLINLLIERIDVLPDGIDIAWREIGWKELAGELAPDTIGSEMLEVERSQ |
| 235 | MKGESELDKKAAIYIRVSTQEQATEGYSIQAQTDRLTKYVEAKDFILYKKYIDAGYSASKLERPAM |
| | QELIQDVQSKKVDVVIVYKLDRLSRSQKDTMYLIEDIFRPNDVELISMQESFDTSTAFGSATVGMLS |
| | VFAQLERKSISERMITGRVERAKKGFYHTGGQDRPPAGYQFNSDNHLIINEYEAAAIKDLFRLYND |
| | GLGKSSISEYLKKNYPGKNKWLPSSIDRMLKNSLYIGKVKFSGVEYDGIHEPIIDEVTFYKTQKEIAR |
| | RKQSNTKRYNYVALLGGLCECGICGAKMANRRAVGRKGKVYRYYRCYSKKGSPKHMMKTDGCS |
| | SKAQQQFIIDEAVINNLKNIDVEAELKRRSAPQTNTSLISSQIESIDKQINKLIDLFQVDSMPLDVISEK |
| | IDKLNKEKQSMEKLLERKNRLDKTELQHRFDVLKSFDWDNSSIESKRVVIEMLVQKVIIHDNSIEIIL |
| | VE |
| 236 | MKTIHKLARPQLPEPPKLKVAAYARVSTSSNEQLASLQTQITHYENHIQNNDQWEYVGVYYDEGIS |
| | GTKVEKRDGLHRLIKDAELGKIDLILTKSISRFSRNTVDCLNLVRKLTDIGVTIFFEKENINTGDMESE |
| | LLLSILSSLAESESYSHSENMKWANRKRMAKGIFKTVPPYGYQRKGADFYLIPDEAKVIEQIFKWAL |
| | EGVSAYQVAKRLNEKNIFTRKGSKWQASGINNILHNIVYTGTMLHQRYFNDDQFRKKKNNGELPM |
| | YRIDNNHPPIISWEDYERVQELITLRANAKGTSKGSQKYSQRYAFTKRIICDKCGCNYKRVHTAGK |
| | GNTKVVKWSCTGHLKNKDGCDALPITDESLKTAYLTMLNKLILGHTIVLEPLINTPVEGKASKQEL |
| | EKLSIEITKIDEKLEVLASLNASGVVSTKTSLEEQGRLQMELNKLQEKQHKIMESVNGTSTQRIQLEQ |
| | LHQFTKRSEMLTEWDEDLFLRFAERIVVYSRQEVSFELKCGLLLKERLEA |
| 237 | MKVAIYCRVSTLEQKEHGYSIEEQERKLRSYCDINDWNVKDVYVDAGFSGAKRDRPELQRMMND |
| | IKRFDLVLVYKLDRLTRNVRDLLDLLEIFEQNNVAFRSATEVYDTSTAMGRLFVTLVGAMAEWER |
| | ETIRERTQMGKLAALKKGIMLTTPPFYYDRVDNKFVPNKYKEVVLFAYEEALKGKSAKSIARKLN |
| | NSDIPPPNNRKWEDRSITRALRSPFTRGHFEWGGVYLENNHEPIITEEMYEKVKDRLEERTNTKKIK |
| | HVSIFRSKLVCPVCDSKLTMNTHKVTLKDRVYYNKHYYCNNCKETPNLKPVYIRSEEVERVFYEY |
| | LQHQDLTEYDIVEDKEEKEVAIDINKVMQQRKRYHKLYANGLMNEDELAELIEETDIAIEEYKKQS |
| | ENEEVKQYDTEDIKQYKNLLLEMWDISSDEEKAEFIQMAIKNIFIEYVLGKNDNKKKRRSLKIKDIE |
| | FY |
| 238 | MKTAIYLRKSRADLEAEARGEGETLAKHRSTLLKIAKEMNLNVLAVREEIVSGESLVKRPEMLALL |
| | EEIEDNKYDVVLCMDMDRLGRGGMKEQGIILETFKRSNTKIMTPRKTYDLNDEWDEEYSEFEAFM |
| | ARKELKIITRRMQRGRIASVEAGNYLGTHAPFGYDIHRLNKRERTLTMDPEEASVVRMIFDWYANE |
| | DMGASAIRNKLNDLGYKSKLGNEWNPYSILDILKNNVYIGKVTWQKRKEVKRPDAVKRSCARQD |
| | KSDWIIADGKHEPIIPESLFEQVQEKLNSRYHVPYNTNGIKNPLAGIIKCSKCGYSMVQRYPKNRKE |
| | TMDCKHRGCENKSSYTELIEKRLLEALKEWYINYKADFEAHKQGDKLKETQVIQMNEAALRKLEK |
| | ELVDVQKQKNNLHDLLERGVYTVDMFLERSQVISDRINEITSTMENLKKEIKTEIKKEKVKKDTIPQ |
| | VEHVLDLYFKTDDPKKKNSLLKSVLEKAVYKKEKWQRLDDFELVLYPKLPQDGDI |
| 239 | MKQIAIYIRKSVKGDENSISLEAQTEIIKHYFKGENNFIIYKDDGFSGGNTNRPAFQKLMADAVENK |
| | FDTIACYKLDRIARNTLDFLTTFNLLKEYNIDLICVEDKYDPSTPAGRLMMTLLASLAEMERENIKQ |
| | RVSDSMLNLAKQGRWTGGTPPFGYKVITLDGGKYLEIEDKNNIKYIFNEFINGKSIIKLGNEFNCNK |
| | KKISRILHNITYLQSSKDASIYLKQILGYEVIGESNGYGYLPYGNYKVVNGKKIKNTDGLKIACISRH |
| | EAIIDLNTFIKVQEKLKTFEGKKAPRISTKSFLAQMVQCTCGSNMLIVLGHKKKDGSRKLYFSCPNK |
| | CGNNFATVKEIEDDTLTVLKNVDFFNKIRQNNTNLNKDNSKIKSTILKELEEKKKLLDGLVNKLAL |
| | VDSSLANVLIEKMESLNIDIKNLQNKIDLLEKEEIASSYNKEDFNLKEESRKHFIEQFENMDTKERQN |
| | AIRGVINKIIWTGKNIIIS |
| 240 | MGEETDYNPADWIDLFCRKSQAVKSKASRGRKQELSISAQETLGRRVAALLGKQVRHVWKEVGS |
| | ASRFRRKGARTDQDQALAAVVKGEVGALWCYRLDRWDRRGAGAILHIIEPEDGIPRRILFGWNEE |
| | TGRPELDSSNKRDRGELIRSAERAREETEVLSERIKNTKDHQRANGEWVNARAPYGLEVVLVETLD |
| | EEGDLYDERRLRVSAELSGDPKGRTKAEIARLWHTLPVTDGLSLRSIAERLSDEGVPNPSGTAGWA |
| | FATGRDIINNPAYAGWQTTGRQEGQNQRRRVFRDENGDKLSVMAGEALVTDEEQLAAKEAVQGE |
| | EGIGVPNDGSEHSVKAKHLMTDASYCESCEGSMPWAGTGYGCWKTKSGQRAACEKPAFVARKA |
| | AEEYIGKRWQDRLIHAEPDDPILIEVAKRYRAAKNPKTSEHESEVLDALARAETALKRVWADRKG |
| | GLYDGPSEEFFKPDLDEATERVTAIQSELERVRGGSNKVDVSWIFDPDLVRHTWERADEKTRRMLL |
| | RLAIDEIWISKAAYQGQPFDGDSRITINWHGESPARRRVKTRKLPSGKVVPLIRPQKGK |
| 241 | MKVAAYCRVSTDQEEQLSSYENQVNYYREFISKHEDYELVDIYADEGISATNTKKRDAFNRLIQDC |
| | RAGKVDRILVKSISRFARNTLDCIKYVRELKELGVGVSFEKENIDSLDSKGEVLLTILSSLAQDESRSI |
| | SENATWGIRKKFERGEVRVNTTKFMGYDKDDNGRLIINPQQAETVKFIYEKFLDGYSPESIAKYLN |
| | DNEIPGWTGKANWYPSAIQKMLQNEKYKGDALLQKTITVDFLTKKRVQNDGQVNQYYVENSHEA |
| | IIDKDTWELVQLELERRKAYREEHQLKSYIMQNDDNPFTTKVFCAECGSAFGRKNWATSRGKRKV |
| | WQCNNRYRVKGQIGCQNNHIDEETLEKAVVIAVELLSENVDLLHGKWNKILEENRPLEKHYCTKL |
| | AEMINKTSWEFDSYEMCQVLDSITISEDGQISVKFLEGTEVDL |
| 242 | MNVAAYCRVSTDQDEQLSSYENQVNYYRDYISKHEDYELVDIYADEGISATNTKKRDAFNRLIQD |
| | CRAGKVDRILVKSISRFARNTLDCIKYVRELKDLGIGVT1AEKENIDSLDSKGEVLLTILSSLAQDESRS |
| | ISENATWGIRKRFERGEVRVNTTKFMGYDKDKDGNLIINREQAKVVRYIYEQFLKGYTPESIARDL |
| | NDQEVPGWSGKANWYPSSILKMLQNEKYKGDALLQKTYTVDFLTKKRTENDGQVNQFYVANNH |
| | EGIIDHEMWETVQLEIARRKAFREEHGIPFYHLQNEDNPFMTKVFCAECGDAFGRKNWTTSRGKR |
| | KVWQCNNRYRVTGVMGCSNNHIDEEMLEKAFMKAVSILNDHKTDVLDKLERLSKGDNLLHKHY |
| | AKFMNQLLDLDHFDSTIMCEILDNITISESGEIRISFLEGTQVDL |
| 243 | MKAAIYIRVSTQEQIENYSIQAQTEKLTALCRSKDWDVYDIFIDGGYSGSNMNRPALNEMLSKLHEI |
| | DAVVVYRLDRLSRSQRDTITLIEEYFLKNNVEFVSLSETLDTSSPFGRAMIGILSVFAQLERETIRDR |
| | MVMGKIKRIEAGLPLTTAKGRTFGYDVIDTKLYINEEEAKQLRLIYDIFEEEQSITFLQKRLKKLGFK |
| | VRTYNRYNNWLTNDLYCGYVSYKDKVHVKGIHEPIISEEQFYRVQEIFARMGKNPNMNRDSASLL |
| | NNLVVCGKCGLGFVHRRKDTVSRGKKYHYRYYSCKTYKHTHELEKCGNKIWRADKLEELIIDRV |
| | NNYSFASRNVDKEDELDSLSEKLKIEHVKKKRLFDLYISGSYEVSELDAMMADIDAQINYYEAQIE |
| | ANEELKKNKQIQENLADLATVDFDSLEFREKQLYLKSLINKIYIDGEQVTIEWL |
| 244 | MKVAAYCRVSTDQEEQLSSYENQVNYYRDYISKHEDYELVDIYADEGISATNTKKRDAFNRLIQD |
| | CRAGKVDRILVKSISRFARNTLDCIKYVRELKELGVGVTFEKENIDSLDSKGEVLLTILSSLAQDESR |
| | SISENATWGIRKKFERGEVRVNTTKFMGYDKDENGRLIINPGQAETVKFIYEKFLEGYSPESIAKYL |
| | NDNEIPGWTGKANWYPSAIQKMLQNEKYKGDALLQKTFTVDFLTKKRVQNDGQVNQYYVENSH |
| | EAIIDKDTWELVQLELARRKDFREEHQLKAYIIQNDDNPFTTKVFCKACGSAFGRKNWTTSRGKRK |
| | VWQCNNRYRVKGQIGCQNNHIDEETLEKAVVMAVELLSENVDLLHGKWNKILEENRPLEKHYCT |
| | KLAEMINKPLWEFDSYEMCQVLDSITISEDGQISAKFLEGTEVDL |
| 245 | MIIYLNKIILGGSSLTTGIYIRVSTEEQAKEGYSIANQKEKLIAFCESQGWSSYKIYSDEGYSAKDMK |
| | RPALQEMFNDMTQGVIKIILVYKLDRLTRSVRDLYTMLETFDKHDCKFKSATEVYDTTTAMGRLFI |
| | TLVAALAQWERENTAERVRVVMENNVKNGKWKGGTLAYGYQLKNGNIVINEDEAATVSFIFNKI |
| | KFTGPLAIVRELIKKNIPTRTGSDWHVDTIRGIITNPFYIGYQRFNDSLKQYKGSVKQQKLYKSSHESI |
| | ISEDEFWEVQEILNARKTHGSKKSTSTYYFSTVLTCGVCGASMCGHLSGNKKTYRCNKKKTSGNC |
| | DSSLILESTIVNWLLTNLESISKMLINNTITNTKGTITKEKHVNDFQKELKKITKLKEKHKTMYENDII |
| | DIAELIEQTNKYRHREKEIKEIIHNIDKQDEKNEILKATLYNFNDAWAAATEPERKFLINSIFQNISIHA |
| | IGVHTRTKPRDIVISSIY |
| 246 | MKAAIYIRVSTQEQIENYSIQAQTEKLTALCRSKDWDVYDIFIDGGYSGSNMNRPALNEMLSKLHEI |
| | DAVVVYRLDRLSRSQRDTITLIEEYFLKNNVEFVSLSETLDTSSPFGRAMIGILSVFAQLERETIRDR |
| | MVMGKIKRIEAGLPLTTAKGRTFGYDVIDTKLYINEEEAKQLQMIYDIFEEEKSITSLQKRLKKLGF |
| | KVKSYSSYNNWLTNDLYCGYVSYADKVHTKGVHEPIISEEQFYRVQEIFSRMGKNPNMNRDSASL |
| | LNNLVVCGKCGLGFVHRRKDTVSRGKKYHYRYYSCKTYKHTHELEKCGNKIWRADKLEELIIDRV |
| | NNYSFASRNVDKEDELDSLNEKLKIEHTKKKRLFDLYISGSYEVSELDAMMSDIDAQINYYEAQIEA |
| | NEELKKNKKIQENLADLATVDFNSLEFREKQLYLKSLINKIYIDDEQVTIEWL |
| 247 | MVIVAYAVYVRVSSDKDEQVSSVENQIDICRYWLENNGFEWDENAVYFDDGISGTAWLERHAIQL |
| | VLEKARKKEIDTVVFKSIHRLARDLKDALEIKEILLGHGVRLITIEEGYDSHYEGKNDMKFEMYAM |
| | FASQLPKTLSVSITAALAAKVRRGGYTGGFVPYGYEIIDGKYAINEEEAALVREIFELYAQGFGYIKI |
| | ANTINDKGARTRKGAPWTFSTLSKMIKNPAYKGTYIMQKYGTVKVNGRKKKVINPKEKWVIFEGH |
| | HPAIISHELWEKVNNKDPNKFKKKRRVSTTNELRGITVCAHCGTAMSKRNSINVSKNGRETEYSYM |
| | ICNWSRITARRECVRHVPIHYKDLRALVLSKLKEKERELDKEFCSDENQLQVKLRKLKKDINDLKF |
| | KRERLLDLYLEDERIDKDTFTIRNAKIEKEIGLKEMEIRKASNIEIQMKEKQEVRDAFALLEESKDLH |
| | SVFQKLIKRIEVAQDGAIDIYYRFEE |
| 248 | MWASAGATTYPATVTRQRETQDGVKAGWSRTVALDHTDDADTAQALPLRAAEYVRMSTEHQQY |
| | STENQRDRIREYAARRGLEIVRTYADEGKSGLRIDGRQALQQLIHDVESGTANFQMILVYDVSRWG |
| | RFQDADESAYYEYICKRAGIQVAYCAEQLILNDGSPVSTIVKGVKRAMAGEYSRELSAKVFAGQCR |
| | LIELGFRQGGPAGYGLRRILVDQHGLMKGDLQRGEHKCLQTDRVILMPGPESETRIVNLIYDWFIDE |
| | ALNEYEIAARLNGMRIRTELGREWTRATVREVLTNEKYIGNNVYNRVSFKLKKTRVVNPPEMWIR |
| | KDGAFQSIVPSETFYTAQGIMRARARRYSFEELIERLRNLYRSRGFLSGVVIDETEGMPSASVYAYR |
| | FGSLIRAYQTVGFTPGRDYRYVETNRFLRQLHPEIVAETEKKITDLGGTVSRDPATDLLTVNTEFTA |
| | CIVLSRCQAHDNGRNHWKVRFDTSLLPDITVAVRLNHENAAALDYYLLPRLDFGQLRIHLADHNPI |
| | EFESYRFDTLDYLYGMAERARLRRGA |
| 249 | MLRAAIYIRVSTKLQEEKYSLRAQTTELRRYVEQQRWRLVDEFQDIESGGKLHKKGLNALLDIVEE |
| | GKIDVVVCIDQDRLSRLDTISWEYLKSTLRENKVKIAEPGTIVDLGDEDQEFVSDIKNLIAKREKKAL |
| | VKRMMRGKRQRMREGKGWGQAPYEYYYDKKEEQYKLKKEWAWVIPFIDRLYLEEQLGMRSITD |
| | ELNKISKTPSGIMWNEHLVHTRLTTKAYHGVQEKTFANGEVIAAENIFPKLRTKETWEKIQIERNKR |
| | GNQYKVTSRKRNDLHLLRRTYFVCGECGRKISLAAHGTKEAPRYYLKHGRKLRLADGSVCDVSIN |
| | TVRVEGNIIQAIKDIVTSKELAKQYVNLENEKEEITQLEQNIKNNEQIIQKHTTKNEKLIDLYLDNHL |
| | TKEQLNKKQHEIKNITENLQTQLKRDKAKLETLKSDSWSYDFLSELFESINFFDSDFSPLERAMLMG |
| | NIFPEGIVYRDHIILKANVGGLNFDVKVLVNEDPFPWHYSKSNSKQK |
| 250 | MTVGIYIRVSTEEQAREGFSISAQREKLKAYCVSQDWTDYKFYVDEGKSAKDTNRPYLKLMLDHI |
| | QQGLIDVVLVYRLDRLTRSVKDLYKLLDLFDKNNCIFRSATEVYDTGSATGRLFITLVAAMAQWE |
| | RENLGERVTMGQVEKARQGQYSAPAPFGFKKQDETLVKDKKQGYILMDMIDKVKKGWSIRQIAK |
| | YLDQSYLPIRGYKWHIATILSILHNPALYGALRWKDELNETSHEGYLTKEEFEELQNILYSRQNFRK |
| | RQIESAHIFQMKLVCPQCGNRLGCERSVYFRKKDQKNVESLHYRCQSCALNERPSISVSEKKLEKA |
| | LLLFMKNVKFDLEPVVKEEKNETTEIQNAIVKIERQREKFQKAWASDLMTDEEFTARMSETRKAHE |
| | NFTKRLSEIQRATPLPIDIKKAKKLVNEFKINWAYLNTEEKREFVQSFIEKIEFTKKDQNPHILNVSFY |
| 251 | MKTLKYAVYVRVSTDRDEQVSSVENQIDICRYWLEKNGYEWDPNAVYFDDGISGTAWLERHAMQ |
| | LILEKARRNELDTVVFKSIHRLARDLRDALEIKEILIGHGIRLVTIEENYDSLYEGGNDIKFEMFAMF |
| | AAQLPKTLSVSISAAMQAKARRGEVIGKPGLGYDVIDKRLVINEKEAEVVREIFDLSKKGFGYKKIA |
| | SILNDKGIYTKSGQLWSDTTIAKVLKNQKYKGDLVLNRYKTVKVDGRKKRIYTPKDRLTIIEDHYP |
| | AIVSKELWNEVNNNRVSQKKVKQNMRNEFRGMIFCNHCGGSITVKYSGKCSKKNKKEWVYLKCS |
| | NFLRFNQCVNFNPIYYDEIREIHYRLKQKEKELEIHFNPKIHEKREAKSIEIKKDIKLLKAKKEKLIDL |
| | YVEGLIDKDVFSKRDLNFENEIKEQELELLKLMDQNKRVNEEQQIKKAFSMLDEEKDMHEVFKILI |
| | KKITLSKDKYVEIEYTFSL |
| 252 | MYELKYAVYVRVSTDKDEQVSSIQNQIEICRYWIEKNGFEWDENSIYKDEAVSGTAWLERHAMQL |
| | ILEKVRRKELDTVVFKSIHRLGRDLRDALEIKEILLGHGVRLVTIEEGYDSYYEGKNDLKFEMYAMF |
| | ASQLPKTVSVSVSAALAAKVRRGEYTGGIVPYGYKIVDQKYTINEDEAELVKKMYELYDNGLGYM |
| | KIADAINDMGVPSRTGKLWAYPSIRAIITNAAYKGDYIMQKYAEVKVDGRKKMIINPKEKWVVFE |
| | NHHPAIITRDLWDKVNNPKTDKKTKRRVAINNELRGLACCAHCGTPLALQQRMYKNKEGETRYY |
| | CYLICGRYKRMGARGCVKHSGLQYSDLRLFVLQKLKEKENDLEKVFNLNDTDKHQEKQKKLRKE |
| | KKELEIKRERLLDLYLDGGPIDKETFTKRDKNFEKIIKEKELEILKLDDVKALVVEQQKVKEAFELLE |
| | ESKDLYSTFKKLITRIEVNQDGVINIVYRFEE |
| 253 | MLKRAALYIRVSTDQQAKHGDSLDAQIATLKDYVSTQDNLTIIDTYIDDGISGQKLYRDEFQRLLED |
| | IKKNRIDIILFTKLDRWFRNLRHYLNIQEILDNSGVTWLAVSQPFFNTDTAYGRSFVNQSMSFAELE |
| | AQMASERIKAVFENKIRKGEVVTGSVPFGYKICDKKLIPNENAPIAKDIFKHYSIHNSIRLTVEYLFN |
| | EYDITRSSRTIKHMLRNRKYIGEVSGNKNYCPPIVDKETFEKVQNLLDKNISSIAKRTYIFSGLVVCS |
| | CCGKKMTGRYRKRKYIKKDGTVMYYTKKVYRCNGNTYKRNKCPNKINIPEEILEEYLLYNIKADA |
| | ENFEAKQKKIAVSAPEKNNNSKVLKKIERLKKAYLNEVISLDEYKKDRKELEQMIVQVKPKETIVF |
| | KSNWFKKNIESTYRDFDEEEKRFVWRSVLKNLIVDPHGKITINFLTKN |
| 254 | MKKVAIYTRVSTLEQANEGYSIEGQEQRLKAYCQVHDWDNFEFFVDAGQSASNTKRAGLQNLLN |
| | RLDEFDLVLVYKLDRLTRSVRDLMSLLDTFEEKDVKFRSATEVFDTTSAIGKLFITLVGAMAEWER |
| | STITERTTQGRRIATEKGVYTTVPPFFYDKIEGKLYPNDKKEIVDYIVSRAKAGVSIRGITEELNNSIY |
| | NPPKGKRWDKSVISYVLTSPVSRGHTHIGDVYVENTHEPVISEEDYTIYMQSISQRTHSRGIKHTAIF |
| | RGKLTCPNCAHSLTLNTSKRTKRDGSVDYDERYICDRCRSDKSAENITIQSKEVERAFIDFIQHGEIE |
| | VNVEDTEEQEEQSVIDVDKIKRQRKKYQQAWAMDLMSDEEFQSLIKETDDLLDQHNRQQLRKKE |
| | NKDNHKQIEATHDLILNLWDKMASNDKEDLINASISNIDYNFYRGHGHGKNRTPNSMSVTHIDYK |
| | V |
| 255 | MYELKYAVYVRVSTDRDEQVSSIENQIDICRYWIEKNGYEWDENSIYKDEAVSGTAWLERRAMQL |
| | ILGKARKKELDTVVFKSIHRLGRDLRDALEIKEILLGHGVRLVTIEEGYDSYYEGKNDLKEEMYAM |
| | FASQLPKTLSVSISAALAAKVRRGEYTGGTVPYGYKIVDKKYVINQEEAEIVREMYELYDNGLGYL |
| | RISNALNDVGKYKRSGKLWTYSAVKLIITNPMYKGDYVMGRSTEVKVDGRKKRIQEPREKWVVF |
| | ENHHPAIIERPLWDKINNPKINKKIKRRVAVTNELRGIARCIHCGSPFVLHTYKYKNKEGEELNYGY |
| | LTCGTYKLTGGRGCVKHSGLRYERLRSLVLRKLKEKERDLEKVFKLNDKDKHQEKQKKLRKEKK |
| | ELEIKRERLLDLYLDGGSIDKETFTKRDANFAKNIKEKELEILKLDDVKALIVEQQKVKDAFKLLED |
| | SENLYPVFKKLIAGIDISQNGAVDIRYRFEE |
| 256 | MKSKALVGARVSVYSDSKVSHQAQRESGHRWCQANGAEVLDEFEDLGVSAIKVSTFERPDLGAW |
| | LTPERSHEWDTIVWAKVDRAWRSMRDGLAFMHWAEDNRKRVVFADDGLELDYRNGRKKGDMQ |
| | AVITDMFMLLLSMFAQIEGERFVQRSLSAHGELKTTDRWQAGTPPFGYLTVDRPSGKGKGLAKNP |
| | DQQEILHEMARLFLEGWSYNRLAIWLNDNQIKTNHNLSVTAKAQKTGKSPKKPLSDRPWQDGTVK |
| | KILTSPATQGFKVINMQPDPEKRKHGIDPDYQIASDPVTGEPIRMADPTFDPETWAKIQDKAAERTA |
| | KPRDKTKWSNPMLGVVYCNCGAAFTRISKEDRNYFYFRCGRERGQACKDRTVRGDFLESTIREFFL |
| | QGHLAHRRVTQRKFVPGNDRSEEFEQIQTSIRNMRRNYEKGYYKGEEDEYEAKMDGLVAKRDRIE |
| | SEGVVIRGGYVTEDTGRTWGDLFSESEDWSVIQEAVKDAGIRLMVEGTYPLIVRVDDPNERDGIPY |
| | FSVEMKRAPDLRSNQYRIWAAIQKDPEANDTVIGSRLGVHPVTVGRWRKRMPADGIDPKPEPQYW |
| | IEPFGGTPDPGESHPGDAAA |
| 257 | MITTNKVAIYVRVSTTNQVEEGYSIDEQKDKLSSYCDIKDWNVYKVYTDGGFSGSNTDRPALESLI |
| | KDAKKRKFDTVLVYKLDRLSRSQKDTLHLIEDVFIKNGIEFLSLQENFDTSTPFGKAMIGLLSVFAQ |
| | LEREQIKERMQLGKLGRAKSGKSMMWAKTSYGYDYHKETGTVTINPAQALTIKFIFESYLRGRSIT |
| | KLRDDLNEKYPKHVPWSYRAVRTILDNPVYCGFNQYKGEIYPGNHEPIISKEEYDKTQSELKIRQRT |
| | AAENVNPRPFQAKYILSGIAQCGYCGAPLKIMLGVKRKDGSRLKKYECHQRHPRTLRGVTTYNDN |
| | KKCDSGFYYKDKLEASVLKEISKLQDDADYLDKIFSGDNTETIDRESYKKQIEELSKKLSRLNDLYI |
| | DDRITLEELQSKSAEFISMRGTLETELENDPALRKNKRKADMRKLLNAEKVFSMDYENQKVLVRR |
| | LINKVKVTAEDIVINWKI |
| 258 | MKITNKVAIYVRVSTTSQVEEGYSIDEQKAKLSSYCDIKDWNVYKIYTDGGFSGANTDRPALEGLI |
| | KDAKRKKFDTVLVYKLDRLSRSQKDTLYLIEDIFIKNNIAFLSLQENFDTSTPFGKAMIGLLSVFAQL |
| | EREQIKERMQLGKIGRAKAGKSMMWARTSYGYDYHRGTGTITVNPAQALAVKFIFESYLRGRSIT |
| | KLRDDLNENYPKHVPWSYRAVRAILDNPVYCGFNQFKGEVYPGNHEPIITEEVYNKTKAELKIRQR |
| | TAAENVNPRPFQAKYILSGIGQCGYCGAPLKIILGVKRKDGSRFKKYECHQRHPRTLRGITTYNDNK |
| | KCDSGFYYKDDLETYVLTEISKLQDDAGYLDKIFSEDSAETIDRESYKRQIEELSKKLSRLNDLYIDD |
| | RITLEELQNKSAEFINMRATLETELENDPALRKGKRKADMRELLNAEKVFSMDYESQKVLVRGLIN |
| | KVRVTAEDIVIKWKI |
| 259 | MKVAVYCRVSTLEQANGGHSIEEQERKLKSFCDINDWSIYDTYVDAGYSGAKRDRPELQRLMKDI |
| | NKFDLVLVYKLDRLTRNVRDLLDLLEIFEKNDVSFRSATEVYDTTTAMGRLFVTLVGAMAEWERE |
| | TIRERTQMGKLAALRKGIMLTTPPFYYDRVDNKFVPNKYKDVILWAYDEAMKGQSAKAIARKLN |
| | NSDIPPPNNTQWQGRTITHALRNPFTRGHFDWGGVHIENNHEPIITDEMYEKVKDRLNERVNTKKV |
| | KHTSIFRGKLVCPNCSARLTLNSHKKKSNSGYIFAKQYYCNNCKVTPNLKPVYIKEKEVIKVFYNY |
| | LKRFDLEKYEVTQKQNEPEITIDINKVMEQRKRYHKLYASGLMQEDELFDLIKETDQTIAEYEKQN |
| | ENREVKQYDIEDIKQYKDLLLEMWDISSDEDKEDFIKMAIKNIYFEYIIGTGNTSQKNNSLKITSIEFY |
| 260 | MKVAIYTRVSTLEQREKGHSIDEQERKLRSFCDINDWTVKDVYVDAGFSGAKRDRPELTRLLDDIS |
| | EFDLVLVYKLDRLTRSVRDLLDLLEVFENNNVAFRSATEVYDTTTAIGRLFVTLVGAMAEWERETI |
| | RERSLMGKRAAIKKGMILTAPPFYYDRVNNTYIPNQYKDVVLDVYNKVKKGYSIAHIARLYNNSD |
| | VKPPNDNKEWTTRMLMHALRNPVTRGHYQWGEIYIEDSHEPIITDEMYNTIIDRLDKHTNTKVVAH |
| | TSVFRGKLICPNCGYALTLNSNKRKRKNDTIVYKTYYCNNCKTTKGMKPHHITETETLRVFKDHLS |
| | KIDLKQYETQEKEKQSHVTIDLSKVMEQRKRYHKLYASGMMQENELFELIKETDEMIEEYEKQRK |
| | QVDVKEFDIGKIKEIKDVLLKSWDIFTLEDKADFIQMSIKAINIEYTKLKRGKSSNSMKIKDIEFY |
| 261 | MTVGIYIRVSTEEQAAEGYSISAQRERLKAFCVAQDYADYKFYVDEGISGRNTKRPQFKKLMGDIK |
| | AGHIKVLLVYRLDRLTRSVRDLHNILDKLEKYNCVFRSATEIYDTFTAMGRMFITIVAAIAEWESAN |
| | LGERVSMGQIEKARQGEWAAQAPYGFYKDENHKLHIDDQQIKAIKIMIQKVREGLSFRQLSIYMDS |
| | TEHKPKRGYKWHIRTLMDLMQNPVLYGAMYFKGTVYENTHQGIMDKKEFDQLQKLITSRQNYKT |
| | RNVTSHFVYQMKIVCPDCGSRCTSERSVWKRKTDGSTQVRNSYRCQVCALNHRDITPFNVREFTV |
| | DEALMEFMDNFPLTPDDKPQEKTDDESLELKQELKRIENQRGKYQRAWATDLVTDEEFKIRMDES |
| | RSRMEEIQVMLKEMKCEVHEEVDIERYKEIAQNFNINFENLSPKERREFVQMFIESVEIEILERTKAK |
| | GFRNQRIRVSSVHFY |
| 262 | MSDSLIRRLRCAVYTRKSTDEGLDQEYNSIDAQRDAGHAYIASQRAEGWIPVADDYDDPAYSGGN |
| | MDRPAIKRLMADIEAGKIDIVVIYKIDRLTRSLTDFARMVDVFERHGVSFVSVTQQFNTTTSMGRL |
| | MLNILLSFAQFEREVTGERIRDKIAASKRKGMWMGGIPPIGYDVVNRRLVLNDGEAKLVRHIFRRF |
| | VEIGSSTLLVKELRLDGVTSKAWTTQDGKVRKGRPIDKALIYKLLHNRTYLGELRHRDQWYPGEH |
| | PSIIDSELWDRVHAILSTNGRARASATRAKVAKVHCLLRGMVFGSDGRALSPISTVKKDGRRYRYY |
| | VPQREKKEHAGASGLPTLPAAELEAAVLDQLRAILRSPGLIGDMLPRAIALDPSLDEAMVTVAMTR |
| | LDAIWDQLFPAEQTRIVNLLVEKVIVSPDDLEVRLRANGIERLVLELRPATDGGAEEVMA |
| 263 | MYRAAEYVRMSTEHQQYSTENQADKIREYAERRGIQIVRTYADEGKSGLSIDGRQALQRLIRDVES |
| | GDADFEMILVYDVSRWGRFQDADESAYYEYICRRAGIQVTYCAEQFENDGSPVSTIVKGVKRAMA |
| | GEYSRELSAKVFAGQCRLIELGYRQGGPAGYGLRRVLVDQTGTFKSELARGEHKSLQTDRVILMPG |
| | PEQEVATVNQIYRWFVDDGLTESEIASRLNAGCVPTDLGREWTRATVRQVLSNEKYIGNNIYNRISF |
| | KLKKHRVVNEPEMWIRKDGAFEAIVPPDIFYTAQGILRARSHRYSNEELLEKLRNLFRQRGVLSGLI |
| | IDEAEGMPSTAAYIHRFGSLLRAYEAVGFTPDRDYRFLEVNQFLRRLHPEIISQTERMILDLGGSVQR |
| | DLATDLLDVNREFTVSMVLARCLVLDNGRRRWKVRFDASLLPDITVAVRLDESNENPLDYYLLPR |
| | LDFGQPGISLADHNRIEYESYRFENLDYLYGMAERYRLRRAA |
| 264 | MKAAIYIRVSTQEQIENYSIQAQTEKLTALCRSKDWDVYDIFIDGGYSGSNMNRPALNEMLSKLHEI |
| | DAVVVYRLDRLSRSQRDTITLIEEYFLKNNVEFVSLSETLDTSSPFGRAMIGILSVFAQLERETIRDR |
| | MVMGKIKRIEAGLPLTTAKGRTFGYDVIDTKLYINEEEAKQLQMIYDIFEEEKSITTLQKRLKKLGF |
| | KVKSYSSYNNWLTNDLYCGYVSYADKVHTKGVHEPIISEEQFYRVQEIFSRMGKNPNMNRDSASL |
| | LNNLVVCGKCGLGYVHRRKDTVSRGKKYHYRYYSCKTYKHTHELEKCGNKIWRADKLEELIIDR |
| | VNNYSFASRNVDKEDELDNLNEKLKTEHTKKKRLFDLYISGSYEVSELDAMMADIDAQINYYNSQI |
| | EANEELKRDKKVQESLAELAAVDFDSLEFREKQIYLKSIINKIYIDGEQVTIEWI |
| 265 | MTKAAIYIRVSTQDQVENYSIEVQRERIRAFCKAKGWDIYDEYIDGGYSGSNLERPGIKKLITDLKNI |
| | DAVVVLKLDRLSRSQRDTLELIEEHFLKNKVDFVSITETLDTSTPFGKAMIGILSVFAQLERETIAER |
| | MRMGHIKRAENGLRGNGGDYDPAGYTRKDGHLVIKKDEAVHIKRAFDLYEQYYSITKVQEVLKE |
| | EGYPIWRFRRYRDILSNTLYIGRVTFSGKEYEGQHEPIISSEQFKRVQALLKRHKGHNAHKAKQSLL |
| | SGLITCSCCGENYVSYSTGKSKAAESKRYYYYICRAKRFPAEYEERCMNKTWSRKKLEEVIISELKN |
| | LTEEKKQTNKKEKKINYEKLIKDIDKKMERLLDLFMNTTNISKGLLEQQMEKLNLEKEKLLLKQQR |
| | SEEESISHEVTLTAIDDAFEILDFKEKQVIINNFIEQIYINQNNVKIIWRF |
| 266 | MKTAIYLRKSRADLEAEARGEGETLAKHRSTLLKIAKEMNLNVLAVREEIVSGESLVKRPEMLALL |
| | EEIEDNKYDVVLCMDMDRLGRGGMKEQGIILETFKRSNTKIMTPRKTYDLNDEWDEEYSEFEAFM |
| | ARKELKIITRRMQRGRIASVEAGNYLGTHAPFGYDIHRLNKRERTLTINSEEASVVRMIFDWYANED |
| | MGASAIRNKLNDLGYKSKLGNEWNPYSILDILKNNVYIGKVTWQKRKEVKRPDAVKRSCARQDK |
| | SDWIIADGKHEPIIPESLFEQVQEKLNSRYHIPYNTNGIKNPLAGIIKCAKCGYSMVQRYPKNRKETM |
| | DCKHRGCENKSSYTELIEKRLLEALKEWYINYKSDFEKYKQDDKLKETQVIQMNEVALRKLEKEL |
| | VDVQKQKNNLHDLLERGVYTVDMFLERSNVVSDRINEITLTMEKLQKEIKTEIKKEKVKKDTIPQV |
| | EHVLDLYFKTDDPKKKNSLLKSVLEKAVYKKEKWQRLDDFELVLYPKLPQDGDI |
| 267 | MVIVAYAVYVRVSSDKDEQVSSVENQIDICRYWLENNGFEWDENAVYFDDGISGTAWLERHAIQL |
| | VLEKARKKEIDTVVFKSIHRLARDLKDALEIKEILLGHGVRLITIEEGYDSHYEGKNDMKFEMYAM |
| | FASQLPKTLSVSITAALAAKVRRGGYTGGFVPYGYEIIDGKYAINEEEAALVKEIFELYAQGFGYIKI |
| | ANTINDKGARTRKGAPWTFSTLSKMIKNPAYKGTYIMQKYGTVKVNGRKKKVINPKEKWVIFEDH |
| | HPAIISHELWEKVNNKDPNKFKKKRRVSTTNELRGITVCAHCGTAMSKRNSINVSKNGTETEYSYM |
| | ICNWSRITARRECVRHVPIHYKDLRALVLSKLKEKEKELDKEFGSDENQLQVKLRKLKKDINDLKF |
| | KRERLLDLYLEDERIDKDTFTIRNAKIEKEIGLKEMEIRKASNIEIQMKEKQEVRDAFALLEESKDLH |
| | SVFQKLIKRIEVAQDGAIDIYYRFEE |
| 268 | MASENDKNHKVRVAQYLRMSTDHQQYSLHNQSEYIKDYAEKNNMEIAYTYDDAGKSGVSIIGRH |
| | SLQQLLSDVEQKKIDIQAVLFYDVSRFGRFQNSDEAAYYSFLFERNGVDLIYCSEPIPTKDFPLESSVI |
| | LNIKRSSAAYHSRNLSEKVFIGQVNLIKLGYHQGGMAGYGLRRLLVDENGIAKEILGFRKRKSIQTD |
| | RVILIPGPKNEIKIVNSIYDLFIDDNMPEFIIAERLNEQNIPAENGTLWTRAKIHQILTNEKYIGNNIYN |
| | KTSSKLKSRLVKNPKNEWVRCDKAYKPIISKKKYNKAQEIIQLRSVHLTNEELLEKLKQKLETNGK |
| | LSGFIIDEDDTGPSSSVYRTRFGGLLRAYTLIGYKPEHDYSYIQINEALRSFYSGIIEDFKGEIIKSNCYI |
| | DEYKYAPMLYINDEFLISVLITKCTHMKSGKLRWKVRFDNSQKADITIVIRMDSQNITPLDFYIIPKIE |
| | NEYSKMCMTETNNIRLDLYRFDNLDKLLQIITRMKVRELYAA |
| 269 | MNKKVAIYVRVSTLEQAESGYSIGEQIDKLKKFADIKEWQVYDVYEDGGFSGSNTTRPALERMISD |
| | AKRKLFDTVLVYKLDRLSRSQKDTLFLIEDVFKVNNIDFVSLNENFDTSTAFGTAMIGILSVFAQLE |
| | REQIRERMKLGLVGRAKSGKAMGWHMTPFGYTYDKKSGNFIIDEVAAGVVKMIFDDYLSGISITK |
| | LRDKLNSEGHIGKDRNWSYRTLRQTLDNPTYTGVVKYDGKTFPGNHEPILTSETFQSVQYELDIRQ |
| | KQAYLKNNNSRPFQSKYILSGIAKCGYCGAPLVSILGNKRKDGTRLLKYQCANRIIRKAHPVTTYN |
| | DNKQCDSGFYMMQNIEAYVINSISELQTNPQKIQEIIKLDNDQPVIDTLYLESELAKISSRLKKLSDL |
| | YMSDLMTLDDLKNRTKELKQTRKNIEAKIFSEENKHGHTKSDIFRSRIDGNNITELDYDKQSMLAK |
| | SLIRKVSVTNETIEISWDF |
| 270 | MRCAIYARVSTEEQAVEGYSISAQKKKLKAYCDAQDWDVVGYYVDEGISAKNTNRPELKRMIEHI |
| | EKGLIDCVLVHRLDRLTRSVLDLYTLLDVELEYDCKFKSATEVYDTTTAIGRLFITIIAALAQWERE |
| | NIGERVRVGQQEKVRQGKYTSPRKPYGYNADHKEGILTIIEEEAKVVRSIYNDYLKGHSATRISKRL |
| | NATKTAGRDYWNEKAVMYILENPLYIGTLRWRKETEHYFEVPNSVPAIIEEEMFNSVQILRESRQES |
| | HPRSQYGSYIFSGILKCPRCGRSLVGNYVVSKKKDGTKIKYKHYYCKGRKLNVCTMGNMSERKLE |
| | QAIIPHILSFYIDATDEDVKLENSNTENEIEQIKSELKIIEKRRKKWQYAWANDHLKDEEFTEFMQEE |
| | NENEKVLTEELYKLKPAENKKLQNEELKNILKDIKLNWANLNDEEKKIFMQIILKKLVIERSDKLHA |
| | YKLEIVEMEFN |
| 271 | MRTVITYLRFSSAIQGAEGADSTRRQNDLFKQWLKKNGDAQIVASFSDEGLSGYKGKHLTGQFGD |
| | MLARIEAGEFPEGTILLVESIDRIGRLEHLETEALMNRILGNGIEIHTLQDGLIYTKDALADDLGISIIQ |
| | RVKAYIAHQKSKQKSFRVSQKWGQRAKLALAGEQRLTKMVPGWIDPETFKLNEHAETVRLIFKLL |
| | LDGESLHNIARHLQSNGIKSFSRRKDANGFSVHSVRTILRSETTIGTLPASQRNDRPAIPNYYEGVVD |
| | IPTFNKAQEILDKNRKAVHLQVTTH |
| 272 | MAVGIYIRVSTQEQASEGHSIESQKKKLASYCEIQGWDDYRFYIEEGISGKNTNRPKLKLLMEHIEK |
| | GKINILLVYRLDRLTRSVIDLHKLLNFLQEHGCAFKSATETYDTTTANGRMSMGIVSLLAQWETEN |
| | MSERIKLNLEHKVLVEGERVGAIPYGFDLSDDEKLVKNEKSTILLDMVERVENGWSVNRIVNYLNL |
| | TNNDRNWSPNGVLRLLRNPVLYGATRWNDKIAENTHEGIISKERFNRLQQILSDRSIHHRRDVKGT |
| | YIFQGVLRCPVCDQTLSVNRFIKKRKDGTEYYGALYRCQPCAKQNKYNFAIGEARFLKALNEYMS |
| | TVEFQTEEDEVSSEKNEREILESQLQQIARKREKYQKAWASDLMSDDEFEKLMVETRETYNECKQQ |
| | LENCKDPVKIDTKYLKEIVFMFHQTFNSLESEKQKEFISKFIRTIRYTIKEQQPIRPDKSKTGKGKQKV |
| | IITEVEFYQ |
| 273 | MKKITKIDGNKGTSIIKPKLRVAAYCRVSTDNDEQLVSLQAQKSHYETYIKANPEWEYVGLYYDEG |
| | ISGTKKENRSELLRMLSDCENKKIDLIITKSISRFARNTTDCLEMVRKLLDLGIYIYFEKENINTQSME |
| | SELMLSILSGLAESESISISENNKWAIQRRFQNGTFKISYPPYGYDNIDGQMVVNPEQAEIVKYIFAEV |
| | LSGKGTQKIADDLNQKGIPSKRGGRWTATTIRGILKNEKYTGDVILQKTYTDSRFNKRTNYGEKNR |
| | YLIENHHEAIISHEDFEAVDAVLNQRAKEKGIEKRNCKYLNRYAFSSKIICSECGSTFKRRIHSSGRK |
| | YIAWCCSKHISNITECSMQFIRDEDIKTAFVTMMNKLIFGQKFILRPLLNGLRSQNNAESFRRIEELET |
| | KIESNMEQSQMLTGLMAKGYLEPALFNKEKNSLETERERFLAEKYQLTRSVNGDFAKVEEVDRLL |
| | KFATKSKMLNAYEDEVFEDYVEKIIVFSREKVGFELKCGITLKERLVN |
| 274 | MAVSRNVTVIPAIKRIGNNKNSESKPKIRVAAYCRVSTDSEEQASSYEIQIEHYTNYIKRNKEWELA |
| | GIFADDGITGTNTKKRDEFNRMIEECMAGNIDMIITKSISRFARNTLDCLKYIRQLKDKNIAVFEEKE |
| | NINTMDSKGEVLLTIMASLAQQESQSLSQNVRLGIQYRYQQGEVQVNHKRFLGYTKDENKQLVIDP |
| | EGAEVVKRIFREYLEGSSLLQIARGLEADGILTAAGKSKWRPETLKKILQNEKYIGDALLQKTYTIDF |
| | LSKKRVKNNGIVPQYYVENSHEPIIPRELFMQVQEEMVRRVNLRGGKGGKKRVYSSKYALSSIVYC |
| | GQCGDIYRRVHWNNRGYKSIVWRCVSRLEEKGSECTAPTINEETLQAAVVKAINELLTKKEPFLST |
| | LQKNIATVLNEENDNTTDDIDRKLEELQQQLLIQAKSKNDYEDVADEIYRLRELKQNALVENAERE |
| | GKRQRIAEMTDFLNEQSCELEEYDEQLVRRLIEKVAVLEDKLVIEFKSGIEIEEEM |
| 275 | MKPRQWAAENTEEKPKLKVAAYCRVSTEMEEQASSYEAQVQHYTDYIQRNPDWELAGIFADEGIS |
| | GTGTKKRDGFNRMIEECKKGDVEYIITKSISRFARNTVDCLQYIRQLKDLHIAVFFEKENINTMDAK |
| | GEVLLTIMASLAQQESQSLSQNTKMGVQYRFQQGQLRINHNHFLGYTKDEDGNLVIEPKEAEVIKR |
| | IFREYLEGSSLQEIANGLMSDGILTGGKRKLWRGEGVRLILRNEKYMGDALLQKTYTTDFLTKKRV |
| | KNDGSYAQQYYVENSHPAIIPRDIFMQVQQELDRRKSMKNKHSQCFSGKYALSGITVCGDCGNAY |
| | RRVHWKNRGTVWRCKSRVDKREHNCSGRTIYEKDLHEAIIKAINETLVDREDFLQQLSENINSVLT |
| | DGLTGRLEELDSKLKELESEIISMAFGGQGYDELATKILALRNERDMVGREIAADANMQQRIDEMG |
| | DFVKNHDTISEYSEVLVRRLIEKVTIFEKDIVVDFKSGVNIAIEI |
| 276 | MKAAIYIRVSTQEQIENYSIQAQTEKLTALCRSKDWDVYDIFIDGGYSGSNMNRPALNEMLSKLHEI |
| | DAVVVYRLDRLSRSQRDTITLIEEYFLKNNVEFVSLSETLDTSSPFGRAMIGILSVFAQLERETIRDR |
| | MVMGKIKRIEAGLPLTTAKGRTFGYDVIDTKLYINEEEAKQLRLIYDIFEEEQSITFLQKRLKKLGFK |
| | VRTYNRYNNWLTNDLYCGYVSYKDKVHVKGIHEPIISEEQFYRVQEIFARMGKNPNMNRDSASLL |
| | NNLVVCGKCGLGFVHRRKDTVSRGKKYHYRYYSCKTYKHTHELEKCGNKIWRADKLEELIIDRV |
| | NNYSFASRNVDKEDELDYLNEKLKIEHAKKKRLFDLYINGSYEVSELDSMMNDIDAQINYYESQIE |
| | ANEELKKNKKIQENLADLATVDFDSLEFREKQLYLKSLINKIYIDDEQVTIEWL |
| 277 | MKAAIYIRVSTQEQVENYSIQAQTEKLTALCRSKDWDVYDTFIDGGYSGSNMNRPALNEMLSKLH |
| | EIDAVVVYRLDRLSRSQRDTITLIEEYFLKNNVEFVSLSETLDTSSPFGRAMIGILSVFAQLERETIRD |
| | RMVMGKIKRIEAGLPLTTAKGRTFGYDVIDTKLYINEEEAKQLQMIYDIFEEEKSITTLQKRLKKIGF |
| | KVKSYSSYNNWLTNDLYCGYVSYADKVHTKGVHEPIISEEQFYRVQEIFSRMGKNPNMNRDSASL |
| | LNNLVVCGKCGLGFVHRRKDTVSRGKKYHYRYYSCKTYKHTHELEKCGNKIWRADKLEELIIDRV |
| | NNYSFASRNVDKEDELDSLNEKLKIEHTKKKRLFDLYISGSYEVSELDAMMSDIDAQINYYEAQIEA |
| | NEELKKNKKIQENLADLATVDFNSLEFREKQLYLKSLINKIYIDDEQVTIEWL |
| 278 | MKVPVWCYARISTLKQIDGFGIQRQINTINQFLQCVELDHRLPFTLDVDNVTQMVAEGKSAFREKN |
| | WNEKTKLGQYRKLVMDGVVKESVLITESIDRLTRLDPYKAVEILSGLINRGTTILEVDTGMTYSRYI |
| | PESLSVLTMQINRANGESKRKSIMMQKSHANRYGKVSKVRPRWFDVVEIDDIKQYRPNETAKAIQR |
| | MYNDYINGIGAAHIVRTYGNTDNGKAWTLVTVLRALSDKRVADDARYPPIIDKDLYDSVQALKAA |
| | TNKKGNTHQKNMLNIFSGMSRCPVCNQSIIVKRNSHGNLFTVCLGKRTNKTCSARSISYFALERPLL |
| | TAIRGLDFSEVYKHEDKNVLTLRDQWIQNERDIAAFRERLNKASRHEKFAILDELEIMNREQEELTI |
| | RLKSVDVPKDIQLTFDDDKLDLDTNYRIELNNRIKKLIQHINIVREDVSKSSYTIYCTIKYWTDVISHL |
| | VIIDVNIKRTGTGGTNTLTTTLRSVSSLNMDGTVSGNPDSDAWEYWKSFLDNLK |
| 279 | MKPRQWAAENTEEKPKLKVAAYCRVSTEMEEQASSYEAQVQHYTDYIQRNPDWELAGIFADEGIS |
| | GTGTKKRDGFNRMIEACQKGDVEYIITKSISRFARNTVDCLQYIRQLKDLHIAVFFEKENINTMDAK |
| | GEVLLTIMASLAQQESQSLSQNTKMGVQYRFQQGQLRINHNHFLGYTKDEDGNLVIEPKEAEVIKR |
| | IFREYLEGSSLQEIANGLMSDGILTGGKRKLWRGEGVRLILRNEKYMGDALLQKTYTTDFLTKKRV |
| | KNDGSYAQQYYVENSHPAIIPRDIFMQVQQELDRRKSMKNKHSQCFSGKYALSGITVCGDCGNAY |
| | RRVHWKNRGTVWRCKSRVDKREHNCSGRTIYEKDLHEAIIKAINETVVDREDFLQQLSENINSVLT |
| | DGLTGRLEELDSKLKELESEIISMAIGGQGYDELVSQIFSLRDERDAVAKQIAANTNLQQRVDEMVV |
| | FVKEHDVINEYSEVLVRRLIEKVTIFEKNIVVDFKSGVRVTVEI |
| 280 | MKAAIYIRVSTQEQIENYSIQAQTEKLTALCRSKDWDVYDIFIDGGYSGSNMNRPALNEMLSKLHEI |
| | DAVVVYRLDRLSRSQRDTITLIEEYFLKNNVEFVSLSETLDTSSPFGRAMIGILSVFAQLERETIRDR |
| | MVMGKIKRIEAGLPLTTAKGRTFGYDVIDTKLYINEEEAKQLRLIYDIFEEEQSITFLQKRLKKLGFK |
| | VRTYNRYNNWLTNDLYCGYVSYKDKVHVKGIHEPIISEEQFYRVQEIFARMGKNPNMNRDSASLL |
| | NNLVVCGKCGLGFVHRRKDTISRGKKYHYRYYSCKTYKHTHELEKCGNKIWRADKLEELIIDRVN |
| | NYSFASRNVDKEDELDYLNEKLKIEHAKKKRLFDLYINGSYEVSELDSMMNDIDAQINYYESQIEA |
| | NEELKKNKKIQENLADLATVDFDSLEFREKQLYLKSLINKIYIDDEQVTIEWL |
| 281 | MKAAIYIRVSTQEQIENYSIQAQTEKLTALCRSKDWDVYDIFIDGGYSGSNMNRPALNEMLSKLHEI |
| | DAVVVYRLDRLSRSQRDTITLIEEYFLKNNVEFVSLSETLDTSSPFGRAMIGILSVFAQLERETIRDR |
| | MVMGKIKRIEAGLPLTTAKGRTFGYDVIDTKLYINEEEAKQLRLIYDIFEEEQSITFLQKRLKKLGFK |
| | VRTYNRYNNWLTNDLYCGYVSYKDKVHVKGIHEPIISEEQFYRVQEIFSHMGKNPNMNKESASLL |
| | NNLVVCSKCGLGFVHRRKDTVSRGKKYHYRYYSCKTYKHTHELEKCGNKIWRADKLEELIIDRVN |
| | NYSFASRNIDKEDELDSLNEKLKIEHTKKKRLFDLYISGSYEVSELDAMMNDIDAQINYYEAQIEAN |
| | EELKKNKKIQENLADLATVDFNSLEFREKQLYLKSLINKIYIDDEQVTIEWL |
| 282 | MKAAIYIRVSTQEQVENYSIQAQTEKLTALCRSKDWDVYDTFIDGGYSGSNMNRPALNEMLSKLH |
| | EIDAVVVYRLDRLSRSQKDTITLIEEYFLKNNVEFVSLSETLDTSSPFGRAMIGILSVFAQLERETIRD |
| | RMVMGKIKRIEAGLPLTTAKGRTFGYDVIDTKLYINEEEAKQLRLIYDIFEEEQSITFLQKRLKKLGF |
| | KVRTYNRYNNWLTNDLYCGYVSYKDKVHVKGIHEPIISEEQFYRVQEIFSRMGKNPNMNKESASL |
| | LNNLVVCSKCGLGFVHRRKDTVSRGKKYHYRYYSCKTYKHTHELEKCGNKIWRADKLEELIIDRV |
| | NNYSFASRNIDKEDELDSLNEKLKIEHAKKKRLFDLYINGSYEVSELDYMMNDIDAQINYYEAQIEA |
| | NEELKKNKKIQENLADLATVDFNSLEFREKQLYLKSLINKIYIDGEQVTIEWL |
| 283 | MRCAIYRRVSTDEQAEKGFSLENQKLRLESFATSQGWEVVEDYVDDGFSGKDTNRPALQRMFSNV |
| | DKFDVILVYKLDRFTRSVKDLNEMLETIKENEIAFKSATESIDTTTATGRMILNMMGTTAQWERETI |
| | SERIKDVFGKLRENGIFSTGHPPYGYRCSGNKSIEIVEEQAEIVRYIYELSKTMGLFKISVELNRKGIK |
| | TRRNNKFGQSAVKRILHNPFYCGYMEVNNKWVPIKNEGYIPIISEEEFKTTQKILTKRNKAQTRSRS |
| | VSYYPFSGIVLCPECQRAMRGDRAKYGDYYYRYYRCVYGRENINCTNRKRIRAEQVDKAFAEYIS |
| | GSFENTTIKLDSKDIKSDIEYELKHLDSKIERLSDIYIEGDITKSKYNEKMNSLLNEKEKLKKDLTSCK |
| | ENVDAEFVRDQINKLESIWHLIDDKTKSESIRSIFDTIKIKQDKNKVTIMDHTLL |
| 284 | MKYAVYVRVSTDKDEQVSSIQNQIEICRYWIEKNGFEWDENSIYKDEAVSGTAWLERRAMQLILG |
| | KARKKELDTVVFKSIHRLGRDLRDALEIKEILLGHGVRLVTIEEGYDSYYEGKNDLKFEMYAMFAS |
| | QLPKTLSVSISAALAAKVRRGEYTGGTVPYGYKIVDKKYVINQEEAEIVREMYELYDNGLGYLRIS |
| | NALNDVGKYKRSGKLWTYSAVKLIITNPMYKGDYVMGRSTEVKVDGRKKRIQEPREKWVVFENH |
| | HPAIIERPLWDKINNPKINKKIKRRVAVTNELRGIARCIHCGSPFVLHTYKYKNKEGEELNYGYLTC |
| | GTYKLTGGRGCVKHSGLRYERLRSLVLRKLKEKERDLEKVFKLNDKDKHQEKQKKLRKEKKELEI |
| | KRERLLDLYLDGGSIDKETFTKRDANFAKNIKEKELEILKLDDVKALIVEQQKVKDAFKLLEDSENL |
| | YPVFKKLIARIDISQNGAVDIRYRFEE |
| 285 | MKTAIYLRKSRADLEAEARGEGETLAKHRTTLLKIAKEKNLNVLAVREEIVSGESLVKRPEMLALL |
| | EEIEDNKYDVVLCMDMDRLGRGGMKEQGIILETFKRSNTKIMTPRKTYDLNDEWDEEYSEFEAFM |
| | ARKELKIITRRMQRGRIASVEAGNYIGTHAPYGYDILRLNKRERTLTINLEEASVVRMIFEWYANED |
| | MGASVITNKLNQLGYKSKLGNDWNPYSVLDMLKNNIYIGKVTWQKRKEVKRPDATKRSCTRQDK |
| | SEWIIADGKHDPIISESLFEKAQEKLNTRYHVPYNTNGLKNPLAGVIRCGKCGYSMVQRYPKNRKK |
| | TMDCKHRGCENKSSYTELIEKRLLEALKEWYINYKADFNKNNQENLSKEKQTIKINQAALRKLEKE |
| | LLDVQKQKNNLHDLLERGVYTVDMFLERSNVVSDRINEITETMENLRKEIKTEITKEKVKKDTIPQV |
| | EHVLDLYFKTDDPQKKNSLLKSVLEKAVYTKEKWQRLDDFKLVLYPKLPQDGDK |
| 286 | MKVALYVRVSTLEQAEEGYSINEQKDKLKKYCEIKDWTIVKEYIDPGRSGSNINRPSMQQLIKDAD |
| | TGLYDAVLVYKLDRLSRSQKDTLYLIEDVFQKNNIHFISLSENFDTSTAFGKAMIGILSVFAQLEREQ |
| | IKERMSMGRVGRAKSGKIMEFNNPAFGYEIDGDNYKVDPLRAEIVKRIYKMYLSGTSINKIKETLNS |
| | EGHIGNKKNWSDTRIRYILSNPTYLGKIRYDGKTYDGKFSPIIDEETFNKTQNELKERQTATYKRFN |
| | MKLRPFQSKYMLSGLLRCGYCGATLFVNSYVYNGKRKLRYNCPSTYKSKQKTRTYKIMDPNCPFK |
| | LVYAKDLEPAVINEIKNLALNPQSIQKPIKKKPDIDVETIQKELAKIRKQQQRLIDLYVISDDVNIDNI |
| | SKKSADLKLQEETLKKQLAPLEEPDNDDKIVAFNEILAQIKDIDSLDYDKQKFIVKKLIKKIDVWND |
| | NKIKIHWNI |
| 287 | MREQKDKLKKYCEIKDWTIVKEYIDPGRSGSNINRPSMQQLIKDADTGLYDAVLVYKLDRLSRSQ |
| | KDTLYLIEDVFQKNNIHFISLSENFDTSTAFGKAMIGILSVFAQLEREQIKERMSMGRVGRAKSGKI |
| | MEFNNPAFGYEVDGDNYKVDPLRAEIVKRIYKMYLSGTSINKIKETLNSEGHIGNKKNWSDTRIRYI |
| | LSNPTYLGKIRYDGKTYDGKFSPIIDEETFNKTQNELKERQTATYKRFNMKLRPFQSKYMLSGLLRC |
| | GYCGATLFVNSYVYNGKRKLRYNCPSTYKSKQKTRTYKIMDPNCPFKLVYAKDLEPAVINEIKNL |
| | ALNPQSIQKPVKKTPDIDVEAIQKELAKVRKQQQRLIDLYVISDDVNIDNISKKSADLKLQEETLKK |
| | QLAPLEEPDNDDKIVAFNEILDQIKDIDSLDYDKQKFIVKKLIKKIDVWNDNKIKIHWNI |
| 288 | MKTAIYLRKSRADLEAEARGEGETLAKHRSTLLKIAKEMNLNVLAVREEIVSGESLVKRPEMLALL |
| | EEIEDNKYDVVLCMDMDRLGRGGMKEQGIILETFKRSNTKIMTPRKTYDLNDEWDEEYSEFEAFM |
| | ARKELKIITRRMQRGRIASVEAGNYLGTHAPFGYDIHRLNKRERTLTINSEEASVVRMIFDWYANED |
| | MGASAIRSKLNDLGYKSKLGNEWNPYSILDILKNNVYIGKVTWQKRKEVKRPDAVKRSCARQDKS |
| | DWIIADGKHEPIIPESLFEQVQEKLNSRYHIPYNTNGIKNPLAGIIKCSKCGYSMVQRYPKNRKETMD |
| | CKHRGCENKSSYTELIEKRLLEALKEWYINYKADEEKHKQDDKLKETQVIQMNEAALRKLEKELV |
| | DVQKQKNNLHDLLERGVYTVDMFLERSNVVSDRITEITSTMENLKKEIKTEIKKEKVKKDTIPQVE |
| | HVLDLYFKTDDPKKKNNLLKSVLEKAVYKKEKWQRLDDFELVLYPKLPQDGDI |
| 289 | MKTAIYLRKSRADLEAEARGEGETLAKHRTTLLKIAKEKNLNVLAVREEIVSGESLVKRPEMLALL |
| | EEIEDNKYDIVLCMDMDRLGRGGMKEQGIILETFKRSNTKIMTPRKTYDLNDEWDEEYSEFEAFMA |
| | RKELKIITRRMQRGRIASVEAGNYLGTHAPYGYDIHRLNKRERTLTINSEEASVVRMIFEWYANED |
| | MGANAIMRKLNELGYKSKLGNDWSPYSILDILKNNVYIGKVTWQKRKEVKRPDSVKRSCARQDK |
| | SEWIIADGKHEPILSESLFEKVQEKLNSRYHVPYNTNGLKNPLAGIIKCGKCGYSMVQRYPKNRKQ |
| | TMDCKHRGCENKSSYTELIEKRLLEALKEWYINYKADEEKNKQDESTKETQIIQMNEATLRKLEKE |
| | LVDVQKQKNNLHDLLERGVYTVDMFLERSNVVSNRINEITETMENLRKEIKTEITKEKVKKDTIPQ |
| | VEHVLDLYFKTDDPQKKNSLLKSVLEKAVYTKEKWQRLDDFKLLLYPKLPQDGDK |
| 290 | MKTAIYLRKSRADLEAEARGEGETLAKHRSTLLKIAKEKNLNVLTVREEIVSGESLVKRPEMLALL |
| | EEIEDNKYDVVLCMDMDRLGRGGMKEQGIILETFKRSNTKIMTPRKTYDLNDEWDEEYSEFEAFM |
| | ARKELKIITRRMQRGRIASVEAGNYLGTHAPYGYDIHRLNKRERTLTINLEEASVVRMIFEWYAHE |
| | DMGANAIMRKLNELGYKSKLGNDWNPYSILDMLKNNVYIGKVTWQKRKEVKRPDATKRSCTRQ |
| | DKSEWIIADGKHDPIIPESLFEKAQEKLNTRYHVPYNTNGLKNPLAGIVRCGKCGYSMVQRYPKNR |
| | KHTMDCKHRGCENKSSYTELIEKRLLEALKEWYINYKADFEKNKQDESTKETQIIQMNEAALRKLE |
| | KELVDVQKQKNNLHDLLERGVYTVDMFLERSNVVSNRINEITETMENLRKEIKTEITKEKVKKDTIP |
| | QVEHVLDLYFKTDDPKKKNSLLKSVLEKAVYTKEKWQRLDDFKLVLYPKLPQDDDK |
| 291 | MRCAIYRRVSTDEQAEKGFSLENQKLRLESFATSQGWEVVEDYVDDGFSGKDTNRPALQRMFSNV |
| | DKFDVILVYKLDRFTRSVKDLNEMLETIKKNEIAFKSATESIDTTTATGRMILNMMGTTAQWERETI |
| | SERIKDVFGKLRENGIFSTGHPPYGYRCSGNKSIEIVEEQAEMVRYIYELSKTMGLFKISVELNGKGI |
| | KTRRNNKFGQSAVKRILHNPFYCGYMEVDNKWVPIKNEGYTPIISEEEFKTTQKILTKRTKAQTRSR |
| | SVSYYPFSGIVLCPECQRAMRGDRAKYGDYYYRYYRCVYGRENINCTNRKRIRAEQVDKAFAEYI |
| | SRSFENTTIKLDSRDIKSDIEYELKHLDSKIERLSDIYIEGDITKSKYNEKMNSLLNEKEKLKKDLTSC |
| | KEHVDAEFVRNQINKLESIWNLIDDKTKSESIRSIFDTIKIKQDKNTVTIMDHTLL |
| 292 | MKCVIYRRVSTDEQAEKGFSLENQKLRLESFATSQGWEVVGDYVDDGYSGKNMERPALKRMFND |
| | VDKFDVILVYKLDRFTRSVRDLNDMMETIKEHDIAFKSATEFIDTTTATGRMILNMMGSTAQWERE |
| | TISERVTDTMYKRAESGLWNGGRIPFGYKQVGRNLIINEEESTIVKEMFDLSLSYGFLGVSLKLNER |
| | GYKTKTGCKWNRTGVRHILMNPIYCGYVRYGNQNNDTKDVVMAKIKQDGFKEIVSKERFDECQRI |
| | FESRKKNAPKPRHGEFNYFSGIFVCPNCGRKLYGVTYQQKDNIYKYYKCSKQSQKFCEGFHISLEV |
| | LDAAFLKELNLILDDVKISPLKKIDPVSIKKEIDEISKKKERIKNLYIDEIISRDEMKEKIEELNIKEKDL |
| | YNTLSEEEQQISESIIRETFENLSQNWKQIPDEIKMYMIRSVFESIEFKVIKKARGRWHKAVIEITDYK |
| | MR |
| 293 | MKTAIYLRKSRADLEAEARGEGETLAKHRSTLLKIAKEMNLNVLAVREEIVSGESLVKRPEMLALL |
| | EEIEDNKYDVVLCMDMDRLGRGGMKEQGIILETFKRSNTKIMTPRKTYDLNDEWDEEYSEFEAFM |
| | ARKELKIITRRMQRGRVASVEAGNYLGTHAPYGYDIHRLNKRERTLTINSEEASVVRMIFDWYANE |
| | DMGASAIRNKLNDLGYKSKLGNDWNPYSILDILKNNVYIGKVTWQKRKEVKRPDAVKRSCARQD |
| | KSDWIIADGKHEPIIPESLFEQVQEKLNSRYHVPYNTNGIKNPLAGIIKCSKCGYSMVQRYPKNRKE |
| | TMDCKHRGCENKSSYTELIEKRLLEALKEWYINYKADEEKHKQDDKLKETQVIQMNEAALRKLEK |
| | ELVDVQKQKNNLHDLLERGVYTVDMFLERSNVVSDRITEITSTMGNLKKEIKTEIKKEKVKKDTIP |
| | QVEHVLDLYFKTDDPKKKNSLLKSVLEKAVYKKEKWQRLDDFELVLYPKLPQDGDI |
| 294 | MRCAIYRRVSTDEQVEKGYSLENQKIRLESFATSQGWEVVGDYVDDGYSGKDTNRPAFKKMFKD |
| | VEKFDVILVYKLDRFTRSVKDLNEMLETIREHDIAFKSATESIDTTTATGRMILNMMGSTAQWERET |
| | ISERIKDVIDKQREQGIWNGGITPYGYRKTDGILSVQEDEAETVRFIFKNVIAYGYIKISKLLNEKGIP |
| | TAKGKGLWIAQSVRNIVKNHYYYGKMNYCNNGREEFAEIKIEGYKPIISKDEFNLAQKATKKRAST |
| | PTRSRSDEIYPFSGIAVCPQCGAKLGGTIVKVRGSKYKYYRCSKRNQNRCNSPAFRDTSLDEAFLKY |
| | LKMPYPDLKVKRVDNLNSSDVIKKEIKKLNSKKDKVKELYIEEFLTKKEFKDKIFTIDNKILELESEL |
| | ENNNQAISDDLYRETLLFMEQTWNGLDDETKAFSLRGLFDSLVFKKTGRSKVEFIDHTLL |
| 295 | MKYAVYVRVSTDKDEQVSSIQNQIEICRYWIEKNGFEWDENSIYKDEAVSGTAWLERRAMQLILG |
| | KARKKELDTVVFKSIHRLGRDLRDALEIKEILLGHGVRLVTIEEGYDSYYEGKNDLKFEMYAMFAS |
| | QLPKTLSVSISAALAAKVRRGEYTGGTVPYGYKIVDKKYVINQEEAEIVREMYELYDNGLGYLRIS |
| | NALNDVGKYKRSGKLWTYSAVKLIITNPMYKGDYVMGRSTEVKVDGRKKRIQEPREKWVVFENH |
| | HPAIIERPLWDKINNPKINKKIKRRVAVTNELRGIARCIHCGSPFVLHTYKYKNKEGEELNYGYLTC |
| | GTYKLTGGRGCVKHSGLRYERLRSLVLRKLKEKERDLEKVFKLNDKDKHQEKQKKLRKEKKELEI |
| | KRERLLDLYLDGGSIDKETFTKRDGNFVKNIQEKELEILKLDDVKALIVEQQKVKDAFKLLEDAEN |
| | LYPVFKKLIARIDISQNGAVDIRYRFEE |
| 296 | MSVAIYVRVSTLEQAESGYSIGEQTEKLKSYCKIKDWDIAKIYTDPGYSGSSLDRPAIQALISDCKAG |
| | FFDAVLVYKLDRLSRSQKDTLYLIEDVFNANNIHFMSLSENFDTSTPFGKAMIGLLSVFAQLEREQI |
| | KERMQMGKLGRAKAGKISAWANVPFGYVKNKDTYDIDPLRSEIVKRIYKDYLSGKSITRIMQDLN |
| | QEGHIGKDTLWSYRTVRQVLDNETYTGRTKYRGQVFNGLHKSIITKDDWDEVQRLLKIRQLDQAK |
| | KSNNPRPFQARYMLSGLLKCVYCGSTLAIAKSHTKDGPLWRYVCPSHNVRKYRNGGSAAHYRIAP |
| | INCKFKFKYMSELESAVIHEVKKIALDPSAVISSQDDQPEIDKAAIKAQLKKIKRQQDKLVDLYLLG |
| | DDLDVDQLHKRADQLKEQAAALRAQLKPSDKNIESFKKTVKDAKEIEKLDYEHQKSIVRMLIDHV |
| | NVGNDGINIFWKM |
| 297 | MLKRAALYIRVSTDQQAKHGDSLDAQIATLKDYVSTQDNLTIIDTYIDDGISGQKLYRDEFQRLLED |
| | IKKNRIDIILFTKLDRWFRNLRHYLNIQEILDNSGVTWLAVSQPFFNTDTAYGRSFVNQSMSFAELE |
| | AQMASERIKAVFENKIRKGEVVTGSVPFGYKICDKKLIPNENAPIAKDIFKHYSIHNSIRLTVEYLFN |
| | EYDITRSSRTIKHMLRNRKYIGEVSGNKNYCPPIVDKETFEKVQNLLDKNISSIAKRTYIFSGLVVCS |
| | CCGKKMTGRYRKRKYIKKDGTVMYYTKKVYRCNGNTYKRNKCPNKINIPEEILEEYLLNNIKADA |
| | ENFEAKQKKIAVSAPEKNNNSKILKKIERLKKAYLNEVISLDEYKKDRKELEQMIVQVKPKETIVFK |
| | SNWFNKNIESTYRDFDEEEKRFVWRSVLKNLLVDPHGKITINFLTKN |
| 298 | MKAAIYIRVSTQEQIENYSIQAQTEKLTALCRSKDWDVYDIFIDGGYSGSNMNRPALNEMLSKLHEI |
| | DAVVVYRLDRLSRSQRDTITLIEEYFLKNNVEFVSLSETLDTSSPFGRAMIGILSVFAQLERETIRDR |
| | MVMGKIKRIEAGLPLTTAKGRTFGYDVIDTKLYINEEEAKQLQMIYDIFEEEKSITTLQKRLKKLGF |
| | KVKSYSSYNNWLTNDLYCGYVSYADKVHTKGVHEPIISEEQFYRVQEIFSRMGKNPNMNRDSASL |
| | LNNLVVCGKCGLGYVHRRKDTVSRGKKYHYRYYSCKTYKHTHELEKCGNKIWRADKLEELIIDR |
| | VNNYSFASRNVDKEDELDSLNEKLKTEHTKKKRLFDLYISGSYEVSELDAMMSDIDAQINYYEAQI |
| | EANEELKKNKQIQENLADLATVDFDSLEFREKQLYLKSLINKIYIDGEQVTIEWL |
| 299 | MKGESKLDKKAAIYIRVSTQEQATEGYSIQAQTDRLTKYVEAKDFILYKKYIDAGYSASKLERPAM |
| | QELIQDVQSKKVDIVIVYKLDRLSRSQKDTMYLIEDIFRPNDVELISMQESFDTSTAFGSATVGMLS |
| | VFAQLERKSISERMITGRVERAKKGFYHTGGQDRPPAGYQFNSDNQLIINEYEAAAIKDLFRLYND |
| | GLGKSSISEYLKKNYPGKNKWLPSSIDRMLKNSLYIGKVKFSGAEYDGIHEPIIDEVTFYKTQKEIAR |
| | RKQTNTKRYNYVALLGGLCECGICGAKMANRRAVGRKGKVYRYYRCYSKKGSPKHMMKTDGCS |
| | SKAQQQFIIDEAVINNLKNIDVEAELKRRSAPQTNTSLISSQIESIDKQINKLIDLFQVDSMPLDVISEK |
| | IDKLNKEKQSMEKLLERKNKLDKTELQHRFDILKSFDWDNSSIESKRVVIEMLVQKVIIHDNSIEIIL |
| | VE |
| 300 | MKAAIYIRVSTQEQIENYSIQAQTEKLTALCRSKDWDVYDIFIDGGYSGSNMNRPALNEMLSKLHEI |
| | DAVVVYRLDRLSRSQRDTITLIEEYFLKNNVEFVSLSETLDTSSPFGRAMIGILSVFAQLERETIRDR |
| | MVMGKIKRIEAGLPLTTAKGRTFGYDVIDTKLYINEEEAKQLRLIYDIFEEEQSITFLQKRLKKLGFK |
| | VRTYNRYNNWLTNDLYCGYVSYKDKVHVKGIHEPIISEEQFYRVQEIFSRMGKNPNMNRDSASLL |
| | NNLVVCGKCGLGFVHRRKDTVSRGKKYHYRYYSCKTYKHTHELEKCGNKIWRADKLEELIIDRV |
| | NNYSFASRNIDKEDELDSLNEKLKIEHTKKKRLFDLYISGSYEVSELDAMMSDIDAQINYYEAQIEA |
| | NEELKKNKKIQENLADLATVDFNSLEFREKQLYLKSLINKIYIDDEQVTIEWL |
| 301 | MTALLQVVEPELWVGYIRVSTWNEEKISPEIQEDALRAWAIRTGRRLADPLVVDLDATGRNFNRKI |
| | QGAIERVERREAKGIAVWRFSRFGRNRVGNNVNLARLESVGGQLESATEPVDARTALGELQREMIF |
| | AFGNYESNRAGEQWRETHEVRLKNQLPATGRARFGYVWHPRRVPDPTAPTGWRLQDERYTLHQE |
| | YASVAEEMFERKLAKPVPQGFNTIGHWLNEELRVTTLRGGLWHTSTISRYMDSGFAAGYLLSHDR |
| | ECTCGYGKDPKQSKCANGRMLYLPGAQPKIIEDDVWEEYKAHRKLTKNKPPRTRKATYTLTGLLR |
| | HGYCRHHISHASATQKGVQVPGHWLVCSRNKNVSKIACPQGINASRKEVEDQVFDWLGRVAPKV |
| | DALPVIPGQTTAPKEDPRVATKRERAWINTELKKVEAALDRLVEDNAMDPDKYPADAFDRVRNKF |
| | VAKKGALTKQLAALGEAEATPQREDFQPLIDSLLAEWESFTNIERNAMLETAIRRVVVHDIRSEDSR |
| | FIKIRTEVHPVWEPDPWEPKKICRGPFGTRAGWLSAALFERPAEFDIEHQAQSEAAPAA |
| 302 | MVDAGQRVLGRIRLSRLTDESTSKERQQEVIEQWSQMNGHTIVGWAEDMDVSRSVDPFDTPALGE |
| | WLTKPEKVEQWDIVATWKLDRLATGSIYLNKMMHWCFKHGKVIVSVTENFDLSTWVGRMIANVI |
| | AGVAEGELEAIKERTKASRKKLVESGRWPGGKAPYGYRPVKLDDGGWALEINPEQEAVILRAAAE |
| | IIDGAAFESVAKRLREEGVPTPRGGTWAPSVLKKMLMNKSLLGHSTYRGETVRDAHGNPVLISDPI |
| | FQLDEWNRLQAAAEARTVAPRRTRQTSPLLGIVKCWECEENLAYKYYKTRHCYYHCRHSGEHTQ |
| | MMRSEDVEKWLEEEFLLKVGDELAQERVYVPAENHRQALDEATKAVDELTALLATVSSDTMRTR |
| | LLGQLGSLDAKISELEKMPSREAGWELREMDYTYRDAWERADTEGKRQLLLRSEITAQIKLTDRSA |
| | NGAGGAGMFHTKLNIPEDILERLAASRD |
| 303 | MEVAAYLRVSTDEQAESGHSLLEQQERLKAYAKVMGWDKPTFYIDDGYSAGSLKRPQLQKLIRDI |
| | ENRKVSILMTTKLDRLSRNLLDLLQIIKFMETHDCNYVSATESFDTSTAAGRMVLHLLGVFAEFER |
| | GRTSERVKDNMTSLARNTNIALSGPCFGFDIIDKQYVLNKKEAKYGLKMVEMTEAGHGTRSIAQW |
| | LNSMNVKTKRGKQWDSTTVRRLLRTETICGTRVINKRKKVNGKTVMRPKEEWIIKENNHEGFISPE |
| | RFKNLQNILDSRKINKQHENETYLLTGILKCGYCGGTMKGSSARVSRGDKKYEYYRYICSSYVKGS |
| | GCKHHAAHREDIENAVIIQIESITNSSNKELQLKVVTSNEDEDVFELKRALESLNKQMMRQIEAYGK |
| | GLIEEEDLERSNKHVKEQRQLLRNQLDSLEQFNTPKALKEKAKILLPDIKSLDRKKAKTTIAQLIDSL |
| | VLTDGELDIVWRI |
| 304 | MKPRQWAAENTEEKPKLKVAAYCRVSTEMEEQASSYEAQVQHYTDYIQRNPDWELAGIFADEGIS |
| | GTGTKKRDGFNRMIEECKKGDVEYIITKSISRFARNTVDCLQYIRQLKDLHIAVFFEKENINTMDAK |
| | GEVLLTIMASLAQQESQSLSQNTKMGVQYRFQQGQLRINHNHFLGYTKDEDGNLVIEPKEAEVIKR |
| | IFREYLEGSSLQEIANGLMSDGILTGGKRKLWRGEGVRLILRNEKYMGDALLQKTYTTDFLTKKRV |
| | KNDGSYAQQYYVENSHPAIIPRDIFMQVQQELDRRKSMKNKHSQCFSGKYALSGITVCGDCGNAY |
| | RRVHWKNRGTVWRCKSRVDKREHNCSGRTIYEKDLHEAIIKAINETLVDREYFLQQLSENINSVLT |
| | DGLTGRLEELDSKLKELESEIISMAIGGQGYDELATKILALRNERDMVEREIAADANMQQRIDEMG |
| | DFVKNHDTISEYSEVLVRRLIEKVTIFEKDIVVDFKSGVNIAIEI |
| 305 | MKAAIYIRVSTQEQVENYSIQAQTEKLTALCRSKDWDVYDIFIDGGYSGSNMNRPALNEMLSKLHE |
| | IDAVVVYRLDRLSRSQRDTITLIEEYFLKNNVEFVSLSETLDTSSPFGRAMIGILSVFAQLERETIRDR |
| | MVMGKIKRVESGLPLTTAKGRTFGYDVVDTKLYVNKEEAQHLQLIYDIFEEEKSITFLQKRLKKLG |
| | FKVKSYSSYNKWLMNDLYIGYVSYGDKVHVKGVHEPIISEEQFYRVQEVFSRMGKNPNMNKESSS |
| | LLNNLIVCEKCGLSFVHRVKDTVSRGKKYHYRYYSCKTYKHTHELEKCGNKTWRADKLEEIIIDRV |
| | KNYSFATRNVDKEDELDSINAKLKVEHLKKKRLFDLYINGSYEVAELDKMMADIDAQINYYNSQIE |
| | ANEELKRNKKVQESLAELATVDFDSLEFREKQIYLKSIINKIYIDGEQVTIEWI |
| 306 | MKYAVYVRVSTDKDEQVSSIQNQIEICRYWIEKNGFEWDENSIYKDEAVSGTAWLERRAMQLILG |
| | KARKKELDTVVFKSIHRLGRDLRDALEIKEILLGHGVRLVTIEEGYDSYYEGKNDLKFEMYAMFAS |
| | QLPKTLSVSISAALAAKVRRGEYTGGTVPYGYKIVDKKYVINQEEAEIVREMYELYDNGLGYLRIS |
| | NALNDVGKYKRSGKLWTYSAVKLIITNPMYKGDYVMGRSTEVKVDGRKKRIQEPREKWVVFENH |
| | HPAIIERPLWDKINNPKINKKIKRRVAVTNELRGIARCIHCGSPFVLHTYKYKNKEGEELNYGYLTC |
| | GTYKLTGGRGCVKHSGLRYERLRSLVLRKLKEKERDLEKVFKLNDKDKHQEKQKKLRKEKKELEI |
| | KRERLLDLYLDGGSIDKETFTKRDANFAKNIKEKELEILKLDDVKALIVEQQKVKDAFKLLEDSENL |
| | YPVFKKLIAGIDISQNGAVDIRYRFEE |
| 307 | MKAAIYIRVSTQEQIENYSIQAQTEKLTALCRSKDWDVYDIFIDGGYSGSNMNRPALNEMLSKLHEI |
| | DAVVVYRLDRLSRSQRDTITLIEEYFLKNNVEFVSLSETLDTSSPFGRAMIGILSVFAQLERETIRDR |
| | MVMGKIKRIEAGLPITTAKGRTFGYDVIDTKLYINEEEAKQLRLIYDIFEEEQSITFLQKRLKKLGFK |
| | VRTYNRYNNWLTNDLYCGYVSYKDKVHVKGIHEPIISEEQFYRVQEIFSRMGKNPNMNRDSASLL |
| | NNLVVCGKCGLGFVHRRKDTVSRGKKYHYRYYSCKTYKHTHELEKCGNKIWRADKLEELIIDRV |
| | NNYSFASRNVDKEDELDSLNEKLKIEHTKKKRLFDLYISGSYEVSELDAMMSDIDAQINYYEAQIEA |
| | NEELKKNKKIQENLADLATVDFNSLEFREKQLYLKSLINKIYIDDEQVTIEWL |
| 308 | MTGKQVTVIPMKPKKWVADNTEEKPKLKVAAYCRVSTEMEEQASSYEAQVQHYTDYIQKNPDW |
| | ELAGIFADEGISGTDTKKRAEFNRMIDACKNGEIEYIITKSISRFARNTVDCLQYIRKLKELKIAVFFE |
| | KENINTMDAKGEVLLTIMASLAQQESQSLSQNTKMGVQYRFQQGQLRINHNHFLGYTKDEDGNLV |
| | VEPKEAEIIKRIFREYLEGSSLQDIAKGLMDDGILTGGKRKLWRAEGVRLILRNEKYMGDALLQKTF |
| | TVDFLTKKRVKNDGSYAQQYYVENSHPAIIPKDIFTQAQQELDRRKSMKNKNSQCFSGKYALTGIT |
| | ICGDCGNVYRRVHWKNRGTVWRCKSRVDKREHNCNGRTIYEKDLHQGILQAINETLIDRDVFLQQ |
| | LTDNINSVLTDGLTEQLAGLDEQLKDLESEIISVAIGGQGYDELASQIFSLRDERDAVAKQIAANTNL |
| | QQRVDEMVVFVKEHDVINEYSEVLVRRLIEKVTIFEKNIVVDFKSGVRVTVEI |
| 309 | MKLLVTYIRWSTKEQDSGDSLRRQTILIDAFYSKHKNDYYLLPAHRYVDKGKSGFHQQHKAQGSD |
| | FRRMFENVMSGAIPEGSLIVVENFDRFSRADIDTAIDDVRQILRKGVSILTLGDGELYDKSALTDPVK |
| | LIKHIIIAERAHQESLVKQKRIAQVWNHKTQLARELKKPMGKQAPGWLELSEDGSHYIVDEDKASL |
| | VNIIYDKRLSGMSMFAICKWLNEQGYPTINQRKVRISKTKKPDGNWSALSVKHILTSRSVLGYLPA |
| | KISTEDRKTVLREEIEGFYPQIVTDSKFYAVQRLLEETGKGKTSSGEHWLYVNILKGLIRCRCGLVM |
| | TPTGIRKPVYQGTYRCNGNKESRCSYGTVSRKLLDTQLCSRLFSKLSQLHDEATDTAKLDELQRRL |
| | NTVDSELEKLTETLIQLPNITQIQEALRVKQEEKDELIVQLSREKGKRPISDVL |
| 310 | MVLVYKLDRLTRSVRDLLDLLEIFDQNNVAFRSATEVYDTTNAMGRLFVTLVGAMAEWERATITE |
| | RTLYGKEGALEGGKFLGHVPFYYDLVDNKLIPNENRKYVDYIIKRLKENISATQIGKELSNMKNTP |
| | VKFNKTMVIQILHSPTAHGHTKYGKFFKENTHEPVITQEDYNTAIKILSTRRHTYKQNHASIFRGKIA |
| | CPNNCGRFLHLNVNKIKRADGSYYLRQYYKCDKCSREKKPSTIIRYDMMQEAFMKYLNNLSFDTIE |
| | PPENNDDEEEFEIDIAKVMRQREKYQKAWAMDLMTDDEFKARMKETDKLLEEASEKEVENNELEF |
| | EQVIKIQKLLQKSWKNLSEDKKEDLIAATIDKIQIEIIRGNKTVNSPNEVKIKDVSFLL |
| 311 | MRTNEHNFHNIEEEIKHVAVYLRLSRGEDESELDNHKTRLLNRCELNNWSYELYKEIGSGSTIDDRP |
| | VMQKLLTDVEKNLYDAVLVVDLDRLSRGNGTDNDRILYSMKVSETLIVVESPYQVLDANNESDEE |
| | IILFKGFFARFEFKQINKRMREGKKLAQSRGQWINSVTPYGYKVNKTTKKLTPSEEEAKVVIMIKDF |
| | FFEGKSTSDIAWELNKRKIKPRRATEWRSSSIANILQNEVYIGNIVYNKSVGNKKPSKSKTRVITPYR |
| | RLPEEEWRRVYNAHQPLYSREEFDRIKQYFESNVKSHKGSEVRTYALTGLCKTPDGKTLRVTQGK |
| | KGTDDDLYLFPKKNKHGDSSIYKGISYNVVYETLKEVIVQVKDYLDSVLDQNENKDLVEELKEEL |
| | MKKEDELETIQKAKNRIVQGFLIGLYDEQGSIELKVEKEKEIDEKEKEIEAIKMKIDNAKTVNNSIKK |
| | TKIERLLSDVQSAESEKEINRFYKTLIKEIIVDRTDENEAKIKVNFL |
| 312 | MTLPDIPSTFHGSAHAGEPWIGYIRVSTWKEEKISPELQRTAIEQWAARTGRRIVDWIVDLDESGRH |
| | FKRKIMGGIERIERREVRGIAVWRYSRFGRNRTGNAANLARVEAVGGLLESATEPVDASTAIGRFA |
| | RGMYMEFAAFESDRAGEQWKETHEHRLAAKLPATGRPRFGYVWHRRRVPDPTAPSGIRLQDERY |
| | ALHPDHASVVEELYERKIEDHDGFNSLVHWLNEDLAIPTMRGKAWGVSSVSRYLDSGFAAGFLRT |
| | HDKTCPCGYSSGTRSGCPDNRFIYLPGAQPRIIDPDQWEAYKEHRKTIKATPPRARKATYTLTGLLR |
| | HGYCRFHMSAASYTSHGKQLRGHLLVCSRHKYANRVDCPKGISVKREYVEGEVLTWLKREAAPG |
| | VGVGSSATVHRAEPVEDPRARVQRERGRLQAELSKIEGALDRLVADNAMNPEKYPADSFARVRDQ |
| | FAGKKGSIMKALAELGEVETTPTREEYVPLMLDLIEAWPHMDAIERNAVLRQLVRRIVCHDIRAEG |
| | SRWIETRVEVHPVFEPDPWAPIVGEVVARKDEPAEVDDRADAVTLF |
| 313 | MNKVAIYVRVSTSVQAEEGYSIDEQIDKLKSYCQIKDWTVYDVYKDGGFSGGNINRPALEKMIIDA |
| | KKKRFDTVLVYKLDRLSRSQKDTLYLIEDVFSKNDISFLSLQENFDTSTPFGKAMVGLLSVFAQLER |
| | EQIKERMQLGMIGRAKSGKPMMFTNVSFGYTYSPKTQQLTINQAEAVIVKQIFNEFLGGMSPLRLM |
| | AYLNENNILRNGKEWNYQGIQRILRNPVYIGKIKYNNVIYPGLHEPIIDEESYYKAQKLLDARQDEM |
| | RVKGKNRQFKAKYMLSGTAKCGYCGAPLRIKIGNKRLDGTRLKVYQCCNRYPRKYAVVTYNDN |
| | KKCNSGNYQKEDLEQYVIAEIRKLQLKPEKIDKLFNKVSKIDTVQINKQIASIDKKINRLNDLYLND |
| | MIDIDKLKADAEKFKEQKRVLEKELDKDLKIQEQEKNKEDFKKTIGFKDVTKLDYEEQSFIVKSLID |
| | KILVKKGLIKILWKI |
| 314 | MQRVAIYMRVSTDQQAKHGDSLREQQETLDEYIKRNKNLKVVDKYIDGGISGQKLNRDEFQRLLD |
| | DVKNDQIDLILFTKLDRWFRNLRHYLNTQEILEKHNVSWNAVSQQYYDTTTAYGRTFIAQVMSFA |
| | ELEAQMTSERIKSVFSNKIQQGEVVSGKVPLGYKIENKRLVPTSDKDIVIDLFDYYVRVGSLRKTTT |
| | YLEEKHGIVRDYQSVRKLLTNEKYIGKLRNNTNYCEPIIDKDIFETVQLRLSQNVKTSGSHDYIFRGL |
| | VRCADCDGSMSCSTLKSKYIKKTDGEVSYYIRSCYRCTRRRNNPTRCKNKKTYYERALERYLLDNI |
| | QTNIAMHVRTLKKEVTKKDSVKRKKDALFVKIERLKKAYLNEIIELDEYKRDRELLENEIASLKEPK |
| | INKNIAPLKKVLSDDFFEKYEKASINQKNELWRSIIESIEVSVDGNITINFLP |
| 315 | MLKRAALYIRVSTDQQAKHGDSLDAQIATLKDYVSTQDNLTIIDTYIDDGISGQKLYRDEFQRLLED |
| | IKKNRIDIILFTKLDRWFRNLRHYLNIQEILDNSGVTWLAVSQPFFNTDTAYGRSFVNQSMSFAELE |
| | AQMASERIKAVFENKIRKGEVVTGSVPFGYKICDKKLIPNENAPIAKDIFKHHSIHNSIRLTVEYLFN |
| | EYDITRSSRTIKHMLRNRKYIGEVSGNKNYCPPIVDKETFEKVQNLLDKNISSIAKRTYIFSGLVVCS |
| | CCGKKMTGRYRKRKYIKKDGTVMYYTKKVYRCNGNTYKRNKCPNKINIAEEILEEYLLNNIKADA |
| | ENFEAKQKKIAVSAPEKNNNSKILKKIERLKKAYLNEVISLDEYKKDRKELEQMMIQVKPKETIVFK |
| | SNWFNKNIESTYRDFDEEEKRFVWRSVLKNLIVDPHSKITINFLTKN |
| 316 | MKTAIYLRKSRADLEAEARGEGETLAKHRTTLLKIAKEMNLNVLSVREEIVSGESLVKRPEMLALL |
| | EEIEDNKYDVVLCMDMDRLGRGGMKEQGIILETFKRSNTKIMTPRKTYDLNDEWDEEYSEFEAFM |
| | ARKELKIITRRMQRGRVASVEAGNYLGTHAPFGYDIHRLNKRERTLTINPEEASVVRMIFDWYANE |
| | DMGASAIRNKLNDLGYKSKLGNEWNPYSILDILKNNVYIGKVTWQKRKEVKRPDAVKRSCARQD |
| | KSDWIIADGKHEPIIPESLIALQAQEKLNSRYHVPYNTNGIKNPLAGIIKCSKCGYSMVQRYPKNRKE |
| | TMDCKHRGCENKSSYTELIEKRLLEALKEWYINYKADIALAHKQGDKLKETQVIQMNEAALRKLEK |
| | ELVDVQKQKNNLHDLLERGVYTVDMFLERSQVISDRINEITSTMENLKKEIKTEIKKEKVKKDTIPQ |
| | VEHVLDLYFKTDDPKKKNSLLKSILEKAVYKKEKWQRLDDFELVLYPKLPQDGDI |
| 317 | MKGESKLDKKAAIYIRVSTQEQATEGYSIQAQTDRLTKYVEAKDFILYKKYIDAGYSASKLERPAM |
| | QELIQDVKSKKVDVVIVYKLDRLSRSQKDTMYLIEDIFRPNDVELISMQESFDTSTAFGSATVGMLS |
| | VFAQLERKSISERMITGRVERAKKGFYHTGGQDRPPAGYQFNSDNQLIINEYEAAAIKDLFRLYND |
| | GLGKSSISEYLKKNYPGKNKWLPSSIDRMLKNSLYIGKVKFSGVEYDGIHEPIIDEVTFYKTQKEIAR |
| | RKQSNTKRYNYVALLGGLCECGICGAKMANRRAVGRKGKVYRYYRCYSKKGSPKHMMKTDGCS |
| | SKAQQQFIIDEAVINNLKNIDVEAELKRRSAPQTNTSLISSQIESIDKQINKLIDLFQVDSMPLDVISEK |
| | IDKLNKEKQSMEKLLERKNRLDKTELQHRFDILKSFDWDNSSIESKRAVIEMLVQKVIIHDNSIEIILV |
| | E |
| 318 | MIAAIYSRKSKFTEKGESVENQIEMCKDYLKRNFTSIEDIKIYEDEGFSGKDTNRPEFKKMMEDAKN |
| | KKFSILICYRLDRISRNVADFSNTIEELQKYSIDFISLKEQFDTSSPMGRAMMNIAAVFAQLERETIAE |
| | RIKDNMLELAKTGRWLGGTAPLGYKSEVIEYWNEDGKNKKMYKLATAENEIDIVKLIYKLYFKKR |
| | GFSSVATHLCKNKYKGKNGGEFSRETVRQIVINPVYCTADNKIFKWFKSKGATVYGTPDGIHGLM |
| | VYNKREGGKKEKPISEWVIAIGKHAGIISSDIWLKCQNIIEENKSKISPRSGTGEKFLLSGMIICGECGS |
| | GMSSWSHFNKKTNFMERYYRCNLRNRASNRCSNKMLNAYKAEEYISDYLKELDIDTLKEKYLKN |
| | KKSMATYDSSKQELAKLKNVLEDNNKLIKGLIRKLALLDDDIEIVTMLKNEIENIKKENNEINNNIN |
| | KIKSSLEESDRENKFLKELEQSLLNFKKFYDFVDTSEKRALIKSLISTLVWYSKDEILELNPIGIKPNIS |
| | QGVIKRRT |
| 319 | MKKAIAYMRFSSPGQMSGDSLNRQRRLIAEWLKVNSDYYLDTITYEDLGLSAFKGKHAQSGAFSE |
| | FLDAIEHGYILPGTTLLVESLDRLSREKVGEAIERLKLILNHGIDVITLCDNTVYNIDSLNDPYSLIKAI |
| | LIAQRANEESEIKSSRVKLSWKKKRQDALESGTIMTASCPRWLSLDDKRTAFIPDPDRVKTIELIFKL |
| | RMERRSLNAIAKYLNDHAVKNFSGKESAWGPSVIEKLLANKALIGICVPSYRARGKGISEIAGYYPR |
| | VISDDLFYAVQEIRLAPFGISNSSKNPMLINLLRTVMKCEACGNTMIVHAVSGSLHGYYVCPMRRL |
| | HRCGRPSIKRDLVDYNIINELLFNCSKIQPVENKKDANETLELKIIELHMKINNLIAALSVAPEVTAIA |
| | EKIRVLDKELRRASVSLKTLKCKAVSSLGDFHAIDLTSKNGRELCRTLAYKTFEKII1NTDNKTCDIY |
| | FMNGIVFKHYPLMKTISAQQAISTLKYMVDGEVYF |
| 320 | MRKVAIYSRVSTINQAEEGYSIQGQIEALTKYCEAMEWKIYKNYSDAGFSGGKLERPAITELIEDGK |
| | NNKFDTILVYKLDRLSRNVKDTLYLVKDVFTANNIHFVSLKENIDTSSAMGNLFLTLLSAIAEFERE |
| | QIKERMQFGVMNRAKSGKTTAWKTPPYGYRYNKDEKTLSVNELEAANVRQMFNMIISGCSIMSIT |
| | NYARDNFVGNTWTHVKVKRILENETYKGLVKYREQTFSGDHQAIIDEKTYNKAQIALAHRTDTKT |
| | NTRPFQGKYMLSHIAKCGYCGAPLKVCTGRAKNDGTRRQTYVCVNKTESLARRSVNNYNNQKIC |
| | NTGRYEKKHIEKYVIDVLYKLQHDKEYLKKIKKDDNIIDITPLKKEIEIIDKKINRLNDLYINDLIDLP |
| | KLKKDIEELNHLKDDYNKAIKLNYLDKKNEDSLGMLMDNIDIRKSSYDVQSRIVKQLIDRVEVTMD |
| | NIDIIFKF |
| 321 | MLRPICYERVSSIQQIEGGGGLDDQRSALEGYLDKNAGLFENDRLFIQDRGVSAFKNSNISSESQLGI |
| | FLQDVQNRKYGEGDALIVMSLDRISRRSSWAEDTIRFIVNSGIEVHDISASTVLRKDDPHSKLIMELI |
| | QMRSHNESLMKSVRAKAAWDRKIIEAVQNGTVISNKMPMWLKNVDNRYQVIQEKADLIIRCFEW |
| | YRDGFSTGEIVKRIADPKWQMVTVSRLVRDRRLLGEHKCYNDEVIHNVYPKVIDDDLFLTANRMM |
| | DRVMLEKNKPAEDLLLESDVVQEIFQLYESGLGSGAIVKRLPKGWSTVNVLRVLRDKNVVTQKIID |
| | NLTFERVNQKLSMNGVANRIRKDITIAQDDYITNLFPKILKCGYCGGNVAIHYNHVRTKYVICRNRE |
| | ERKICDAKSIQYIRIEKNILKCVKNVDFQKLMIESTGSETSVLDGLHEELSSLRREENSYSDKINERKL |
| | AGKRVGIHLNDGLTEVQDRIEEIEKEIINAQTVREIPKFDFDMDEVLDPMNIELRAKVRKQLRLVLK |
| | AVKYWMFDKRIFIQLEYFNDVLSHMLVIDNKRGGGDVIYEMSIEERKGERIYTVHENGHAVFIASV |
| | TIGTDIWSLALSRTRTIDSIGNYLSLLAREGFEIFVNEDQIDWF |
| 322 | MYGYNLKPCLTRRNTLKRMEQITPPPISASPLVKVAAYARISMETERTPLSLSTQVSYYQQLIHDTP |
| | GWTFAGVFADSGISGTTTHRPQFQEMLALAREGAIDLILTKSISRFARNTVDLLETVRELKDLGVEV |
| | RFEKENISSTSADGELMLTLLASFAQAESEQISQNVKWRIWKGFEEGKANGFHLYGYTDSADGTDV |
| | QIIEEEAAVVRWIFAQYMKETSCEKMAAQLIADGRVPHLADNKLPGEWVRHILKNPHYTGDLLLG |
| | RWSTPEGRPGRAVRNTGQLPQYLVENAIPAIIDRDTFVAVQTEIARRRELGARANWSIETVALTSKI |
| | KCVSCNCSFVRNVRNPKTQNSISTEHWICTERKKGRKTGCGTCEISDTALKGFIAQVLGIEAFDEDV |
| | FNERIDHIDVQGKDHYTFQYTDGTSSSHTWRPNLKKSSWTPARKAAWGELVRARWAEAKRLGLD |
| | NPRQAPTPPEALAKYRAVAKAEAERLRAERGER |
| 323 | MKVAIYTRVSSAEQANEGYSIHEQKRKLISFCEVNDWNRYEVFSDPGVSGGSMERPSLQKLFDRLE |
| | EFDLVLVYKLDRLTRNVRDLLEMLEVFEKNNIAFKSATEVFDTTSAIGKLFITIVGAMAEWERETIR |
| | ERSLMGSHAAVRSGKYIRAQPFCYDLIDDKLKPNQHAKYIRFMVDKLMIGKSASEVVRQLESKKK |
| | PPGITKWNRKTVLNWIKNPVMRGHTKFGDLLIENTHEPIISEDEYLKLIDIIEKRTYKTKSKHKAIFR |
| | GVLECPQCQSKLHLSRSIKKYDSGKTLEVRRYSCDKCHRDNSVKNISFNESEIEREFINTLLKKGTD |
| | NFKISVPKKKSYDIEDNKVKINEQRANYTRSWSLGYIKDEEYFMLMDETENLLKDIEEKAKSHTDE |
| | KLNEEQIRTVKNLLIKGFKIATLEDKEDLITSSVDVIKFEFIPKKFNKNKPLNTVKINEIQFRF |
| 324 | MVIVAYAVYVRVSSDKDEQVSSVENQIDICRYWLENNGFEWDENAVYFDDGISGTAWLERHAIQL |
| | VLEKARKKEIDTVVFKSIHRLARDLKDALEIKEILLGHGVRLITIEEGYDSHYEGKNDMKFEMYAM |
| | FASQLPKTLSVSITAALAAKVRRGGYTGGFVPYGYEIIDGKYAINEEEAALVREIFELYAQGFGYIKI |
| | ANTINDKGARTRKGAPWTFSTLSKMIKNPAYKGTYIMQKYGTVKVNGRKKKVINPKEKWVIFEDH |
| | HPAIISHELWEKVNNKDPNKFKKKRRVSTTNELRGITVCAHCGTAMSKRNSINISKNGTETEYSYMI |
| | CNWSRITARRECVRHVPIHYKDLRALVLSKLKEKEKDLDKEFGSDENQLQVKLRKLKKDINDLKF |
| | KRERLLDLYLEDERIDKDTFTIRNAKIEKEIGLKEMEIRKASNIEIQMKEKQEVRDAFALLEESKDLH |
| | SVFQKLIKRIEVAQDGAIDIYYRFEE |
| 325 | MYYERSYLRSCQVSTLEQKEHGYSIEEQERKLRSYCDINDWNVKDVYVDAGFSGAKRDRPELKRL |
| | LNDIKHFDLILVYKLDRLTRSVRDLLDLLEVFENNDVAFRSATEVYDTTTAMGRLFVTLVGAMAE |
| | WERETIRERTQMGKLAALKKGIMLTTPPFYYDRVDNKFVPNKYKEVVLFAYEEALKGKSAKSIAR |
| | KLNNSDIPPPNNRKWEDRSITRALRSPFTRGHFEWGGVYLENNHEPVITQEMYNKIKDRLNERVNT |
| | KVVAHTSVFRGKLTCPTCGTKLTMNTNKKKTRNGYTTHKSYYCNNCKITPNLKPVYIKEREVLRV |
| | FYDYLLNLNLEKYEIDEKQSEPEITVDIHKVMEQRKRYHKLYANGLMQEDELFDLIKETDEAIKEYE |
| | SQTENKVEKQFDIEGVKKYKKLLLEMWNVSTLEDKAEFVQMAIKSIEFDYIIDDGPPTSRKHSLKIN |
| | QIIFY |
| 326 | MKPRQWAAENTEEKPKLKVAAYCRVSTEMEEQASSYEAQVQHYTDYIQRNPDWELAGIFADEGIS |
| | GTGTKKRDGFNRMIEACQKGDVEYIITKSISRFARNTVDCLQYIRQLKDLHIAVFFEKENINTMDAK |
| | GEVLLTIMASLAQQESQSLSQNTKMGVQYRFQQGQLRINHNHFLGYTKDEDGNLVIEPKEAEVIKR |
| | IFREYLEGSSLQEIANGLMSDGILTGGKRKLWRGEGVRLILRNEKYMGDALLQKTYTTDFLTKKRV |
| | KNDGSYAQQYYVENSHPAIIPRDIFMQVQQELDRRKSMKNKHSQCFSGKYALSGITVCGDCGNAY |
| | RRVHWKNRGTVWRCKSRVDKREHNCSGRTIYEKDLHEAIIKAINETLVDREYFLQQLSENINSVLT |
| | DGLTGRLEELDSKLKELESEIISMAIGGQGYDELATKILALRNERDMVEREIAADANMQQRIDEMG |
| | DFVKNHDTISEYSEVLVRRLIEKVTIFEKDIVVDFKSGVNIAIEI |
| 327 | MAKELTKTASVAAYLRKSREDADQDDTLARHRKQLIDLVKQRGFENVDWYEEIGSADSIKNRPVF |
| | SDLLKKIENDEYDAVCVVAYDRLSRGNQIESGIISKAFKDTETLLITPTRTYDWSIEGDEMLSEFESM |
| | IARSEYRVIKKRLKQGKINAVKNGRLHSGNVPYGYKWDKNDKTAKIDKEKHEIYRLMVKWFLDEE |
| | YSATEIADKLNELGIPSPSGGSTWYSEVVADILTNDFHRGLVWYGKYRARKNGIGIEKNPDSSSIIM |
| | HKGNHEPMKSDEEHGAIIRRISKLRTFKPGRKLNKNTFKLSGLVRCPRCGKVQVVHTPKNRNPHVR |
| | KCLKKSKTRTTECNNTTGIPEEALYKAIVMKIREYNEVLFSKDSSEKKDEEARTYMNQILSLHEKAI |
| | SKSNKRIEKIKEMYMDEIIDKDEFKSRIDKEKKSILEAENEIRTLKESADYHDEIEHEQRKIKWNHEK |
| | VQEFIESDQGFTPSEINLILKLIISHVSYTMVKNEYGEFDVDLRVNFN |
| 328 | MNKVAVYVRVSTTSQLEEGYSIEEQKAKLESYCDIKDWNIYKIYTDGGFSGSTTDRPALEQLVQDA |
| | QSKLFDTVLVYKLDRLSRSQKDTLYLIEDIFLKNDIEFVSLLENFDTSTPFGRAVIGLLSVFAQLERE |
| | QIKERMQLGKLGRAKSGKSMMWAKTSYGYDYDKETGSMTVNEFEALAVKEIYASYLSGISITKLR |
| | DKMNAEYPKKPAWSYRTIRGILANPVYCGLNQYKGQTFQGTHKAIISLDDFEETQRELKKRQQTA |
| | QERLNPRPFQAKYMLSGLAQCGYCHAPLKVVLGQKRKDGTRTKRYECYQRHPRTTRGVTVYNDN |
| | KKCNSGYYYMDILEHYVLTRIAMLQNDPDKIQEIFSGGTSPVIDKQAIQKQIDSLSLKLSKLNDLYL |
| | DDRITLDELRSKSSDFIKQRAILEEEIKKASTDKQVGRRKKIEKLLDASSVIALMSYDNQKVIVRELIE |
| | KVQVTSDKIVIRWKI |
| 329 | MTVGIYIRVSTQEQANEGYSIGAQKERLIAYCAAQGWNDFKFYIDEGISAKDMNRPELQRLLDDVK |
| | NRRISMILVYRLDRFTRRVKDLYEMLEMLDKHNCSFKSATELYDTSNAMGRMFIGLVALLAQWET |
| | ENLSERIKVALEQKVSDGERVGAIPYGFDLTEDEKLIKNEKSKVVYDMIEKTFNGMSATQLANYLN |
| | KTNDDRTWHVKGVLRILKNPAIYGATRWNDKVYENTHEGIISKSQYKKLQEILNDRSKHHRREVT |
| | GNYLFQGKLSCPTCKKPLAVNRYLRKRKDGTEYQSTIYKCSSCYLKGKKIKQIGEKRFLDALYIYM |
| | KNIDLKGIEITEEPDETKHLTDQLKSLEKKREKYQRAWASDLISDSEFEHRMLETRELFEELKRKLSE |
| | KKKPIQVDIEEIKNVVFTFNQTFHFLTQEEKRMFISRFIKKIDYELIPQPPQRPDRCKYGKDLVTITDV |
| | LFY |
| 330 | MSDSLIRRLRCAVYTRKSTDEGLDQEYNSIDAQRDAGHAYIASQRAEGWIPVADDYDDPAYSGGN |
| | MDRPAIKRLMADIEAGKIDIVVIYKIDRLTRSLTDFARMVDVFERHGVSFVSVTQQFNTTTSMGRL |
| | MLNILLSFAQFEREVTGERIRDKIAASKRKGMWMGGIPPIGYDVVNRRLVLNDGEAKLVRHIFRRF |
| | GEIGSSTLLVKELRLDGVTSKAWTTQDGKVRKGRPIDKALIYKLLHNRTYLGELRHRDQWYPGEH |
| | PSIIDSELWDRVHAILSTNGRARASATRAKVAKVHCLLRGMVFGSDGRALSPISTVKKDGRRYRYY |
| | VPQREKKEHAGASGLPTLPAAELEAAVLDQLRAILRSPGLIGDMLPRAIALDPSLDEAMVTVAMTR |
| | LDAIWDQLFPAEQTRIVNLLVEKVIVSPDDLEVRLRANGIERLVLELRPATNGGAEEVMA |
| 331 | MWQENPPNDASPSSVTYRAAEYVRMSTEHQQYSTENQADKIREYAERRGIQIVRTYADEGKSGLSI |
| | DGRQALQQLIRDVESGQADFNAILVYDVSRWGRFQDADESAYYEYICKRAGIQVTYCAEQFENDG |
| | SPVSTIVKGVKRAMAGEYSRELSAKVFAGQCRLIELGYRQGGPAGYGLRRVLVDQSGTFKGELVR |
| | GEHKSLQTDRVILMPGPEQEVATVNQIYRWFVDDGLTESEIASRLNAGCVPTDLGREWTRATVRQ |
| | VLSNEKYIGNNIYNRISFKLKKHRVVNEPEMWIRKDGAFEAIVPPDIFYTAQGILRARSHRYSNEELL |
| | EKLRNLFRQRGVLSGLIIDEAEGMPSTAAYIHRFGSLLRAYEAVGFTPDRDYRFLEVNQFLRRLHPEI |
| | ISQTERMILDLGGSVQRDLATDLLDVNREFTVSMVLARCLVLDNGRRRWKVRFDASLLPDITVAV |
| | RLDESNESPLDYYLLPRLDFGQPGISLADHNRIEYESYRFENLDYLYGMAERYRLRRAA |
| 332 | MAKVYSYMRFSDPRQATGSSADRQLAYASAWASKHGMELDATLTLRDEGLSAYHETHVKQGAL |
| | GAFLRAIDAGRIPVGSVLIVEGLDRLSRAEPLLAQAQLGQIVSAGITVVTASDGREYNRDGLKAEPM |
| | NLVYSLLVMIRAHEESDTKSKRVKAAVRRQCEAWVAGSYRGRIVSGKDPQWLTWGGDSWQFIPE |
| | RVEAVRFALDAYRSGIGAARLVRLMHEKGMVLSDWGIAAQQVYRLVRLPALRGAKRISIDGEDFM |
| | LEDYYPRLLSDEEFSELETLVGQRYRRRGKDEIVGIVTGIGITRCGYCGTALVAQNLMQRVKADGS |
| | LVDGHRRLHCVSYSKNGGCNAGSCSSVPIEHAVLAYCSDQMNLQRLLEPSSADEELRTRLAEAQQ |
| | GVAEVERQLQRVTDALVADDSGAAPLSFVRKARELEEELERRRSAVRVLERELVAMASSVPVAEA |
| | SKWAELAEQAKSVSNVEAREQARQLVMDTFERIVVYMRGVVPEGRRSKYIDVLLVSRAGQSRWL |
| | RVGRRTGAWSAGGDWNGSAP |
| 333 | MGKNGARVYSYLRFSDPRQATGSSADRQLAYASAWASKHGMELDATLTLRDEGLSAYHETHVK |
| | QGALGAFLRAVDEGRIPAGSVLIVEGLDRLSRAEPLLAQAQLGQIVNAGITVVTASDGREYNREGL |
| | KAEPMNLVYSLLVMIRAHEESDTKSKRVKAAVRRQCEAWVAGSYRGRIVSGKDPQWLAWDGDS |
| | WQFIPERVEAVRFALDAYRSGIGAARLVRLMHEKGMVLSDWGIAAQQVYRLVRLPALRGAKRISI |
| | DGEDFMLEDYYPRLLSDEEFSELETLVGQRYRRRGKDEIVGIVTGIGITRCGYCGTALVAQNLMQR |
| | VKADGSLEDGHRRLHCVSYSKNGGCNGGSCSSVPIERAVLAYCSDQMNLQRLLEPSSAGEDLRPR |
| | LVEAQKVVAEIERQLERVTDALLADDSGAAPLAFVRKARELEEDLERRRSAVRALEQELVAKSAS |
| | APAAGASKWAELAERAKSMVDVDAREQARQLVMDTFETLVVYMRGVIPNPKGRYIDVMMKSRA |
| | GQTRWIRVDRRTGVWKEGADRPTTRRS |
| 334 | MSKARVYSYLRFSDPKQAAGSSADRQIEYARRWAAERNLELDDTLSLRDEGLSAYHQRHVKQGA |
| | LGVFLSAAEGGRIAPGSVLIVEGLDRLSRAEPIQAQAQLAQIVNAGITVVTASDGKEYNRERLRSQP |
| | MDLVYSLLVMIRAHEESDTKSKRVKAALRRQCQQWIDGKWRGIIRSGRDPHWVEIRDGQFALVPE |
| | RVAAVREALALFSRGHGKTKILRTLTERGLSMSNAGNHGTFIYRLVRNPMLMGTRVFEIDKEEFRL |
| | QGYYPALLSPEEFAVLQHLADERKGTRVKGEIPGLLTGLGITHCGYCGAAMVAQNYMGRARKAD |
| | GTPQDGHRRLHCVSDSQNSGCVVAGSVSIVPIERAIMTFCADQMNLTKLIEGDDGSAAVAGRLALA |
| | RQKASGLQAQLERLTTALLADDGNAPPATFLRRARELEEQLSAERRVIESLEREVLASASTTAPAAA |
| | DVWAKLTHGVLALDYESRVRARQLVADTFSRIVIYHAGFRPGEGTEKRIGIQLVAKHGNVRMLDV |
| | DRKSGGWRAAEDFDLRALT |
| 335 | MKGESELDKKAAIYIRVSTQEQATEGYSIQAQTDRLTKYVEAKDFILYKKYIDAGYSASKLERPAM |
| | QELIQDVQSKKVDVIIVYKLDRLSRSQKDTMYLIEEIFRPNDVELISMQESFDTSTAFGSATVGMLSV |
| | FAQLERKSISERMITGRVERAKKGFYHTGGQDRPPAGYQFNSDNQLIINEYEAAAIKDLFRLYNDGL |
| | GKSSISEYLKKNYPGKNKWLPSSIDRMLKNSLYIGKVKFSGAEYDGIHEPIIDEVTFYKTQKEIARRK |
| | QSNTKRYNYVALLGGLCECGICGAKMANRRAVGRKGKVYRYYRCYSKKGSPKHMMKTDGCSSK |
| | AQQQFIIDEAVINNLKNIDVEAELKRRSAPQTNTSLISSQIESIDKQINKLIDLFQVDSMPLDVISEKID |
| | KLNKEKQSMEKLLERKNKLDKTELQHRFDVLKSFDWDNSSIESKRVVIEMLVQKVIIHDNSIEIILVE |
| 336 | MKTTNKVAIYVRVSTTSQVEEGYSIEEQKDKLESYCKIKDWSVYKVYTDGGFSGSNTNRPAIEQLI |
| | KDAQKKKFDTVLVYKLDRLSRSQKDTLHLIEDVFIKNGIEFLSLQENFDTSTPFGKAMIGLLSVFAQ |
| | LEREQIKERMQLGKIGRAKAGKSMMWAKTSYGYDYHRETGTITINPAQALTIKFIFESYLRGRSITK |
| | LRDDLNEKYPKHVPWSYRAVRTILDNPVYCGFNQYKGEIYPGNHESIISKEEYDKTQSELKIRQRTA |
| | AENVNPRPFQAKYILSGIAQCGYCGAPLKIMLGVKRKDGSRLKKYECHQRHPRTLRGVTTYNDNK |
| | KCDSGFYYKDKLEAYVLTEISKLQDNAVYLDKIFSGDNAETIDRESYKKQIEELSKKLSRLNDLYID |
| | DRITLEELQSKSAEFISMRGTLETELENDPALRKNKRKADMRKLLNAEKIFSMDYEGQKVLVRGLI |
| | NKVQVTAEDIVINWKI |
| 337 | MLIQTKIRRFNMKKVFVYHRVSSDQQLDGSGIARQAELLEGYLERTGICAEMDDPAPVVLSDQGVS |
| | AFKGLNISEGELGAWMEQVRNGMWDSSILVVESIDRFSRQNPFDVMGYINALMAHNVAIHDVMA |
| | NIVISRSNSKDLPFVMMNAQRAYDESKYKSDRIRKGWAKKREQAFNKGTIVTNKRPQWIEVENDK |
| | YVLNHKAAVVKEIFALYQTGMGCPTIAKQLQTKEGEQYKFNRPWTGELVHKILTNRRVTGKIFISEI |
| | IRNHDDIENPVTQKKYDMDVYPVVINEEEFELVQELLKSRRPNAGRVTVKKDGQEEVLIKSNLFSGI |
| | ARCTECGGPMYHNVVRAKRTPKKGDPKIEEYRYIRCLNERDGLCENKAMTYETVERFVVEHLLSM |
| | DLNTVIKEQEFNPEIEVIRIQIDQVKDQITKEGANKQVISSQADSLIKISRIWADFFPANTSNQPI |
| 338 | MKLPDTFRSPPPDEEGEAYIGYVRVSTYKEEKISPELQREAILAWAKKTRRRIVKWVEDLDVSGRH |
| | FKRKITKCVEDVEAGTVQGVAVWKYSRFGRDRTGNALWLARLEEVGGQLESATEPVDATTAIGRF |
| | QRGMILEFAAFESDRAGEQWRETHNYRKYTLGLPAQGRARFGYVWHRRFDAATGVLQKERYEPD |
| | PETGPLVASLYHLYVAGTGFATLVIKLNEGGHQTIQGARWTNETLTRHMDSGFAAGLLRVHNPEC |
| | RCRNTGGSCRNKIYIQGAHEELIDWDIWEAYQRRRAVVRASHPRARNSLYTLTGLPSCGGCRWGA |
| | SVTNTSYGGEYRRAFAYRCGLRAKAGATACDGVFIVRTKVEHAVEEWLMDKAARGIDMAPSTGP |
| | GPTLTPIDDQAARARARVSAQADVDRHRAALARLRAEHAELPEDWGPGEYEDAVDVIRKKRAEA |
| | QSILDNLPDADPAPDRAEAQQLIASTAEAWPALDDRQKNALLRQMIRRVVLTRTGRGTADIEVHPL |
| | WEPDPWSKQVSPT |
| 339 | MNVAIYCRVSSQEQANEGYSIHEQERKLKSFCEVNNWKNYKVFVDAGVSGGTINRPAFNNLLANL |
| | DKFDLVLVYKLDRLTRSVRDLLSLLETFEEHGVSFRSATEVFDTTSAIGKLFITIVGAMAEWERSTIR |
| | ERSLFGSHAAVREGNYIRVAPFCYDNIDGKLVPNEHKKVIEYIVKKLLEGVTATEIARRLNNANNYP |
| | PTIKNWSKTTVIRLVNNPVMRGHTKHGDLFIENTHEPIITEHNYKRISERLSSRVNYKKQTHTSVFRG |
| | VLECPQCGHKLHYFKSKLKNKNKTYYSEGYRCDYCRTDKTARNIAITFSEIEREFIEYMSNIRLSEN |
| | YCIEVEPKNEVVKIDINKIMRKRSRFQEAYGDGLMTKEEFKQKMFETQKLIDEYEGMENEKDVDD |
| | HITKEQVQAIQNLFRHIWDSPSVSREDKEEFVRQSIKKIDFDFIPKSKVNKTPNTLKINNIDLHF |
| 340 | MKTIHKLARPQLPEPPKLKVAAYARVSTSSNEQLASLQTQITHYENHIQNNDQWEYVGVYYDEGT |
| | SGTKVEKRDGLHRLIKDAELGKIDLILTKSISRFSRNTVDCLNLVRKLTDIGVTIFFEKENINTGDMES |
| | ELLLSILSSLAESESYSHSENMKWANRKRMAKGIFKTVPPYGYQRKGADFYLIPDEAKVIEQIFKWA |
| | LEGVSAYQVAKRLNEKNIFTRKGSKWQDSGINNILHNIVYTGTMIHQRYFNDDQFRKKKNNGELP |
| | MYRIDNNHPPIISWEDYERVQELITLRANAKGTSKGSQKYSQRYVFTKRIICDKCGCNYKRVHIAGK |
| | GNTKVVKWSCTGHLKNKDGCYALPITDESLKTAYLTMLNKLILGHTIVLEPLINTPVEGKASKQEL |
| | EKLSIEITKIDEKLEVLASLNASGVVSTKTALEEQGRLQMELNKLQEKQHKIMESVNGTSTQRIQLE |
| | QLHQFTKRSEMLTEWDEDLFLRFAELIVVYSRQEVSFELKCGLLLKERLEA |
| 341 | MKPRQWAAENTEEKPKLKVAAYCRVSTEMEEQASSYEAQVQHYTDYIQRNSDWELAGIFADEGIS |
| | GTGTKKRDGFNRMIEACQKGDVEYIITKSISRFARNTVDCLQYIRQLKDLHIAVFFEKENINTMDAK |
| | GEVLLTIMASLAQQESQSLSQNTKMGVQYRFQQGQLRINHNHFLGYTKDEDGNLVIEPKEAEVIKR |
| | IFREYLEGSSLQEIANGLMSDGILTGGKRKLWRGEGVRLILRNEKYMGDALLQKTYTTDFLTKKRV |
| | KNDGSYAQQYYVENSHPAIIPRDIFMQVQQELDRRKSMKNKHSQCFSGKYALSGITVCGDCGNAY |
| | RRVHWKNRGTVWRCKSRVDKREHNCSGRTIYEKDLHEAIIKAINETVVDREDFLQQLSENINSVLT |
| | DGLTGRLEELDSKLKELESEIISMAIGGQGYDELASQIFSLRDERDAVAKQIAANTNLQQRVDEMVV |
| | FVKEHDVINEYSEVLVRRLIEKVTIFEKNIVVDFKSGVRVTVEI |
| 342 | MKAAIYSRKSVFTGKGESVENQIQMCKEYGEKNLGIKEFVIYEDEGFSGGNTKRPKFQELLRDVKK |
| | KKFDTLICYRLDRISRNVADFSTTLELLQDNNISFVSIKEQFDTSTPMGKAMVYIASVFAQLERETIA |
| | ERIRDNMLELAKTGRWLGGQTPLGFKSEKISYFDAEMKERTMYKLSPENKELELVKLIYNKYLETG |
| | SIHLTLKYLLSNSIKGKNGGEFASMSINDILRNPVYVRSNQMVIDYLKDKGMNVCGTANGNGILIY |
| | NKRNSKYKKKDINEWIAAVSKHKGIIPANTWIEVQKTLDKNSSKSTPRQGTSKKSILSGVLKCSRCS |
| | SPMRVTYGRKRKDGTSIYYYTCTMKAHSGKTRCDNPNVRGDYLEKAIIKKLQNLNSDVVIKELEE |
| | YKKQLAATTENSIIKNISKEIEEKKKEMDSLLKQLSKVESPVASEFIISKVDSLGTEIKDLEISLTKTNS |
| | KKKENSNIELNIEIVLQSLKEFNTFFNSVESLKTDELTIQRKRYLLERAVDEITIDGETKKIGIDLWGS |
| | KKK |
| 343 | MELKNIVNSYNITNILGYLRRSRQDMEREKRTGEDTLTEQKELMNKILTAIEIPYELKMEIGSGESID |
| | GRPVFKECLKDLEEGKYQAIAVKEITRLSRGSYSDAGQIVNLLQSKRLIIITPYKVYDPRNPVDMRQI |
| | RFELFMAREEFEMTRERMTGAKYTYAAQGKWISGLAPYGYQLNKKTSKLDPVEDEAKVVQLIFKI |
| | FLNGLNGKDYSYTAIASHLTNLQIPTPSGKKRWNQYTIKAILQNEVYIGTVKYKVREKTKDGKRTIR |
| | PEKEQIVVQDAHAPIIDKEQFQQSQVKIANKVPLLPNKDEFELSELAGVCTCSKCGEPLSKYESKRIR |
| | KNKDGTESVYHVKSLTCKKNKCTYVRYNDVENAILDYLSSLNDLNDSTLTKHINSMLSKYEDDNS |
| | NMKTKKQMSEHLSQKEKELKNKENFIFDKYESGIYSDELFLKRKAALDEEFKELQNAKNELNGLQ |
| | DTQSEIDSNTVRNNINKIIDQYHIESSSEKKNELLRMVLKDVIVNMTQKRKGPIPAQFEITPILRFNFIF |
| | DLTATNNFH |
| 344 | MKGESELDKKAAIYIRVSTQEQATEGYSIQAQTDRLTKYVEAKDFILYKKYIDAGYSASKLERPAM |
| | QELIQDVQSKKVDVIIVYKLDRLSRSQKDTMYLIEDIFRPNDVELISMQESFDTSTAFGSATVGMLS |
| | VFAQLERKSISERMITGRVERAKKGFYHTGGQDRPPAGYQFNSDNQLIINEYEAAAIKDLFRLYND |
| | GLGKSSISEYLKKNYPGKNKWLPSSIDRMLKNSLYIGKVKFSGAEYDGIHEPIIDEVTFYKTQKEIAR |
| | RKQSNTKRYNYVALLGGLCECGICGAKMANRRAVGRKGKVYRYYRCYSKKGSPKHMMKTDGCS |
| | SKAQQQFIIDEAVINNLKNIDVEAELKRRSAPQTNTSLISSQIESIDKQINKLIDLFQVDSMPLDVISKK |
| | IDKLNKEKQSMEKLLERKNKLDKTELQHRFDVLKSFDWDNSSIESKRVVIEMLVQKVIIHDNSIEIIL |
| | VE |
| 345 | MAGAKNITVIPARKRVGNTATPDNKPKLKVAAYCRVSTDSDEQATSYDAQVEHYTEFIRKNFEWE |
| | FAGIYADDGISGTNTKKREEFNRMIEDTMAGKIDMIITKSISRFARNTLDCLKYIRQLKEKNVPVFFE |
| | KENINTMDSKGEVLLTIMASLAQQESESLSKNVKMGLQFRYQNGEVQVNHNWFLGYTKDENGHLI |
| | IDEEQAVVVRRIFREYLQGASLKSIADGLMADGIPTATGNKKWRGDGIRKILTNEKYMGDALLQKT |
| | YTVDVLTKKRVSNNGIVPQYYVENNHEAIIPRQLFMQVQEELLRRAHLKTENGKTKRVYSSKYALS |
| | SIVYCGKCGDLFRRVAWKARGASYNKWRCASRIEKGPKEGCDADAISEVELQNAVVRAINKTLGG |
| | REQFLLQLQHNIEEVLNGDSTATLEYIDQRMAKLQEKLVMCVNKNVEYDVIANEIDALREKKASV |
| | VTKDAEQEMLKKRIDEMRQFLQTQTNRVTEYDEQMVRRLIEKITVFDDKLIFEFKSGMTIELKR |
| 346 | MRNVTKIDQVDLSIFKRLRVAAYCRVSTDSNEQELSLDTQRKHYESYIKANSEWEYAGIYYDDGIS |
| | GTKTAKRDGLLRLVEDCEKGLIDLVITKSISRFSRNTTDCLTLVRKLLNYDVYIIFEKENIHTGSMES |
| | ELMLAILASMAESESRSISENEKWSIKKRFQNGTYVISYPPYGYANVNGEMVIVPEQAEVVKEIFAG |
| | CLAGKSTHVIAKELNEKGVPSKKGGKWTGGTINGILTNEKYIGDALFQKTITDAAFKRKRNYGEEE |
| | QYYCEEHHEAIIDRETFEKAKEAIRQRGLGKGNCSEDISKYQNRYAMSGKIKCGECGRSFKRRYHY |
| | TSHGRSYNAWCCSGHLEDSKSCSMKYIRDDDLKRVFLTMMNKLRFGNDLVLKPLLIAITTDNSKK |
| | NIHSVEEIEKEIAANEEQRNHLSTLLTRGYLERPVFTDAHNKLITEYEHLLAKRDLLYRMDDAGYT |
| | MEQKLKELVDFLNGTEPFTEWDDTLFERFIEKVNVLSRDEVEFEFKFGLRLKERMD |
| 347 | MNTKITPQHQSKPAYIYIRQSTLAQVRHHQESTERQYALRDKALALGWPETAIRVLDRDLGQSGAQ |
| | MTGREDFKTLVADVSMGNVGAVFALEVSRLARSNLDWHRLLELCALTHTLVIDADGCYDAGDFN |
| | DGLILGLKGTMAQAELHFLRGRLQGGKLNKAKKGELRFPLPVGLCYGDDGRIVLDPDDEVRGAVQ |
| | LAFRLFQETGSAYAVVKRFAEEGLRFPKRAYGGAWAGRLIWGRLSHGRVLGLIRNPSYAGIYVSG |
| | RYQYRQRITAQAEVHKHVQPVPKTEWRVHLPDHHDGYITPEEFERNQEHLAQNRTNGEGTVLSGA |
| | AREGLALLQGLLICGGCGRALTVRYQGNGGLYPLYLCSARRREGLATTDCMSMRSELLDNAIGEA |
| | VFTALQPAELELAVTALSELEQRDHAIMRQWHMRIERAEYEVALAERRYQECDPANRLVAGTLER |
| | RWNDAMLHLEAIRTESAQFQSQKALVATSEQKAQVLALARNLPRLWRAPTTSAKDRKRMLRLLIR |
| | DITVERRSATRQALLHIRWQGSACTDITVDLPKPAADAMRYPAAFVEQVRELSQHLPDRQIVAHLN |
| | QEGLRSSTGKSFTLEMVKWIRYRYRIEVTCFKRPDELTVQQLAHRLHVSPHVVYYWIERQVVQAR |
| | KLDGRGPWWIALDAAKERQLDDWVRTSGHLQRQHSNTQL |
| 348 | MTKAAIYIRVSTQDQVENYSIEVQRERIRAYCKAKGWDIYDEYIDGGYSGSNLDRPDIKRLLNDLK |
| | KIDVVVVYKLDRLSRSQRDTLELIEEHFLKNNVDFVSITETLDTSTPFGKAMIGILSVFAQLERETIAE |
| | RMRMGHIKRAENGLRGNGGDYDPSGYTRVDGHLILNPNEAKHIKRAFDLYEQYHSITRVQEVLKE |
| | EGYTIWRFRRYRDVLSNTLYIGQITFAGKTYKGQHEPIVSLEQFKRVQALLKRHKGHNAHKAKQSL |
| | LSGLITCSCCGEKFVAYSTGKSKDIESKRYYYYICRAKRFPSEYDEKCLNKTWSRKKLEEVIFDELK |
| | NLTVKKSASQKKEKKINYEKLIKDIDKKMERLLDLFTNTTNISRQLLETKMDKLNLEKEHLILKQQS |
| | YEQEFSISKDMITTINESLETMDFKDKQIIINTFIQEIHIDHDVVDIIWR |
| 349 | MEINKLKAALYVRVSTTEQANEGYSISAQTEKLTNYAKAKDYQIVKTYTDPGISGAKLDRPALQN |
| | MITDIEKGMIDIVLVYKLDRLSRSQKNTLYLIEDVFLKNKVDFISMNESFDTSTSFGRAMIGILSVFA |
| | QLERDAITERTRMGKIERAKEGKWQGGGNFAPFGYRYENDILKVNEFEKIIVQEMFDLYLEGYGTN |
| | KIAEILGTKYPGKVKSPNLVKGILRNKIYIGKINFAGEIYDGLHETFIDKKIFQNVQEIYGKRANKTY |
| | KGDYNQKGLLLGKIYCAKCGAKYYRQVTGSVKYRYVKYACYSQNRSLSSKTMVKDRNCVNKRY |
| | NAEELEQSTIDKINKLTVAELTSTTNLKLLDNRKTIEKEIKNLESQINKLIDLFQLGNISTELLSSRIDN |
| | LNIQKNNLEIELSKLKKVKTKKEIESKLQTLKDFDWDTETTINKIKMIDEFIDKITINDDEVLIHWRL |
| 350 | MRTVRRIQPIKSPCSPKLKVAAYARVSDSRLHHSLSTQISYYNRLIQAHPDWELVGIYYDEGISGKE |
| | QSNRQGFQNIIKDCDNGKIDRIITKSIARFGRNTVELLTTVRQLRLKNIGVTFEKENIDSLSSEGELML |
| | TLLASVAQEESQNMSENIRWRVQKKFENGMPHTPQDMYGYRWDGEQYQIEPNEAKVIRNVFKWY |
| | LDGDSVQQIVDKLNQEHVLTRLGNPFTVASIREFFKQEAYFGRLVLQKTYREAFSRNPKRNKGQRT |
| | KYIIENAHEPIVTKEYFELVLHEKERRYQLMHQESHLNKGIFRDKIFCSDCGCLMIVKVDSKHVKKT |
| | VRYYCRTRNRFGASSCPCRTLGEKRLLASFKSKLGSVPDKEWVENNIKRIEYDFGHRIIKVTPVKGR |
| | KYPIEIRGGRY |
| 351 | MKKVITIEATPSIIRSSSDDFSLKKRRVAGYARVSTDHEDQATSYESQMRYYSEYINGRDDWEFVK |
| | MYSDEGISGTNTKLRTGFKSMVEDALNGKIDLIITKSVSRFARNTVDSLTTVRQLKEVGVEIYFEKE |
| | NIWTLDSKGELLITIMSSLAQEESRSISENVTWGLRKQFAEGKVHFPYTNVLGFKAGEDGAIVVDQD |
| | EAKTVRYIFQQALIGKSPYHIARDLTEQGIPSPSGKSQWNATTIKRMLRNEKYKGDALLQKTYTIDF |
| | LTKKKNINRGELPQYYVENNHEAIVDRETFDAVQQVLDNKGRKSSTTIFSSKLVCGDCGHFFGSKV |
| | WHSTSKYRRVIYRCNEKYNGSSKCSTPHVTEEEVKQWFVSAVNQVIDNRLEVIDNLSVLLSIGSFEV |
| | IDEQIKNLETDAEVVSQLVANLVSENAIISQDQDKYLKKYNQLTSKYEGIVREIESLELQRMEKSKR |
| | NKELQVFMEFLNNQEGLLTDFDELLWETMVESITINLEKKIFFKFKNGAVATI |
| 352 | MRKVAIYSRVSTINQAEEGYSIQGQIEALTKYCEAMEWKIYKNYSDAGFSGGKLERPAITELIEDGK |
| | NNKFDTILVYKLDRLSRNVKDTLYLVKDVFTANNIHFVSLKENIDTSSAMGNLFLTLLSAIAEFERE |
| | QIKERMQFGVMNRAKSGKTTAWKTPPYGYRYNKDEKTLSVNELEAANVRQMFDMIISGCSIMSIT |
| | NYARDNFVGNTWTHVKVKRILENETYKGLVKYREQTFSGDHQAIIDEKTYNKAQIALAHRTDTKT |
| | NTRPFQGKYMLSHIAKCGYCGAPLKVCTGRAKNDGTRRQTYVCVNKTESLARRSVNNYNNQKIC |
| | NTGRYEKKHIEKYVIDVLYKLQHDKEYLKKIKKDDNIIDITPLKKEIEIIDKKINRLNDLYINDLIDLP |
| | KLKKDIGELNHLKDDYNKAIKLNYLDKKNEDSLGMLMDNLDIRKSSYDVQSRIVKQLIDRVEVTM |
| | DNIDIIFKF |
| 353 | MRKVAIYSRVSTINQAEEGYSIQGQIEALTKYCEAMEWKIYKNYSDAGFSGGKLERPAITELIEDGK |
| | NNKFDTILVYKLDRLSRNVKDTLYLVKDVFTANNIHFVSLKENIDTSSAMGNLFLTLLSAIAEFERE |
| | QIKERMQFGVMNRAKSGKTTAWKTPPYGYRYNKDEKTLSVNELEAANVRQMFDMIISGCSIMSIT |
| | NYARDNFVGNTWTHVKVKRILENETYKGLVKYREQTFSGDHQAIIDEKTYNKAQIALAHRTDTKT |
| | NTRPFQGKYLLSHIAKCGYCGAPLKVCTGRAKNDGTRRQTYVCVNKTESLARRSVNNYNNQKICN |
| | TGRYEKKHIEKYVIDVLYKLQHDKEYLKKIKKDDNIIDITPLKKEIEIIDKKINRLNDLYINDLIDLPK |
| | LKKDIEELNHLKDDYNKAIKLNYLDKKNEDSLGMLMDNIDIRKSSYDVQSRIVKQLIDRVEVTMDN |
| | IDIIFKF |
| 354 | MRKVAIYSRVSTINQAEEGYSIQGQIEALTKYCEAMEWKIYKNYSDAGFSGGKLERPAITELIEDGK |
| | NNKFDTILVYKLDRLSRNVKDTLYLVKDVFTANNIHFVSLKENIDTSSAMGNLFLTLLSAIAEFERE |
| | QIKERMQFGVMNRAKSGKTTAWKTPPYGYRYNKDEKTLSVNELEAANVRQMFDMIISGCSIMSIT |
| | NYARDNFIGNTWTHVKVKRILENETYKGLVKYREQTFSGDHQAIIDEKTYNKAQIALAHRTDTKTN |
| | TRPFQGKYMLSHIAKCGYCGAPLKVCTGRAKNDGTRRQTYVCVNKTESLARRSVNNYNNQKICN |
| | TGRYEKKHIEKYVIDVLYKLQHDKEYLKKIKKDDNIIDITPLKKEIEIIDKKINRLNDLYINDLIDLPK |
| | LKKDIEELNHLKDDYNKAIKLNYLDKKNEDSLGMLMDNLDIRKSSYDVQSRIVKQLIDRVEVTMD |
| | NIDIIFKF |
| 355 | MRKVAIYSRVSTINQAEEGYSIQGQIEALTKYCEAMEWKIYKNYSDAGFSGGKLERPAITELIEDGK |
| | NNKFDTILVYKLDRLSRNVKDTLYLVKDVFTANNIHFVSLKENIDTSSAMGNLFLTLLSAIAEFERE |
| | QIKERMQFGVMNRAKSGKTTAWKTPPYGYRYNKDEKTLSVNELEAANVRQMFDMIISGCSIMSIT |
| | NYARDNFVGNTWTHVKVKRILENETYKGLVKYREQTFSGDHQAIIDEKTYNKAQIALAHRTDTKT |
| | NTRPFQGKYMLSHIAKCGYCGAPLKVCTGRAKNDGTRRQTYVCVNKTESLARRSVNNYNNQKIC |
| | NTGRYEKKHIEKYVIDVLYKLQHDKEYLKKIKKDDNIIDITPLKKEIEIIDKKINRLNDLYINDLIDLP |
| | KLKKDIEELKHLKDDYNKAIKLNYLDKKNEDSLGMLMDNIDIRKSSYDVQSRIVKQLIDRVEVTMD |
| | NIDIIFKF |
| 356 | MLRVALYIRVSTEEQALNGDSIRTQIEALEQYSKENDFNIVGKYIDEGCSATNLKRPNLQRLLRDVE |
| | KDKVDLVLMTKIDRLSRGVKNYYKIMETLEKHKCDWKTILENYDSSTAAGRLHINIMLSVAENEA |
| | AQTSERIKFVFQDKLRRKEVISGTIPIGYKIENKHLVIDKEKKYIVKAIFDEYEKSGSVRTLIETINNLH |
| | GELYSYNKIKNILRNELYIGIYNKRGFYVEDYCEPIISKKQFKQIQRILEKNKKTTPNKNIHYHIFSGL |
| | LKCKECGYTLKGNSSNVGEKLYLSYRCSTFYLNKNCVHNVTHNEKHIENYLLTNLKPQLHKHMVK |
| | LEAQNEKIRRNKKSNKKDEKKKIMKKLDKIKDLYLEDLIDKETYRKDYEKLQSQLDNITEEQESQII |
| | DTSHIKKFLDIDINEMYSDLSRVERRRFWLSIIDYIEIDNNKNITINFI |
| 357 | MQQLIKDADTGLYDAVLVYKLDRLSRSQKDTLYLIEDVFQKNNIHFISLSENFDTSTAFGKAMIGIL |
| | SVFAQLEREQIKERMSMGRVGRAKSGKIMEFNNPAFGYEVDGDNYKVDPLRAEIVKRIYKMYLSG |
| | TSINKIKETLNLEGHIGNKKNWSDTRIRYILSNPTYLGKIRYDGKTYDGKFSPIIDEETFNKTQNELKE |
| | RQTATYKRFNMKLRPFQSKYMLSGLLRCGYCGATLFVNSYVYNGKRKLRYNCPSTYKSKQKTRT |
| | YKIMDPNCPFKLVYAKDLEPAVINEIKNLALNPQSIQKPVKKKPDIDVEAIQKELAKVRKQQQRLID |
| | LYVISDDVNIDNISKKSADLKLQEETLKKQLAPLEEPNDDDKIVAFNEILAQIKDIDSLDYDKQKFIV |
| | KKLIKKIDVWNDNKIKIHWNI |
| 358 | MAVGIYIRVSTQEQASEGHSIESQKKKLASYCEIQGWDDYRFYIEEGISGKNTNRPKLKLLMEHIEK |
| | GKINILLVYRLDRLTRSVIDLHKLLNFLQEHGCAFKSATETYDTTTANGRMSMGIVSLLAQWETEN |
| | MSERIKLNLEHKVLVEGERVGAIPYGFDLSDDEKLVKNEKSAILLDMVERVENGWSVNRIVNYLNL |
| | TNNDRNWSPNGVLRLLRNPALYGATRWNDKIAENTHEGIISKERFNRLQQILADRSIHHRRDVKGT |
| | YIFQGVLRCPVCDQTLSVNRFIKKRKDGTEYCGVLYRCQPCIKQNKYNLAIGEARFLKALNEYMST |
| | VEFQTVEDEVIPKKSEREMLESQLQQIARKREKYQKAWASDLMSDDEFEKLMVETRETYDECKQK |
| | LESCEDPIKIDETYLKEIVYMFHQTFNDLESEKQKEFISKFIRTIRYTVKEQQPIRPDKSKTGKGKQKV |
| | IITEVEFYQ |
| 359 | MRICMYLRKSRADEELEKTLGEGETLSKHRKALLKFAKEKNLNIVEIKEEIVSGESLFFRPKMLELL |
| | KEIENKQYSGVLVMDMQRLGRGNMQDQGIILETFKKSNTKIITPMKTYDLSNDFDEEYSEFEAFMS |
| | RKELKMINRRMQGGRVRSVEDGNYIATNAPYGYDIHWINKARTLKPNQKESEIVKLIFKLYIEGNG |
| | AGTIAKHLNSLGYKTKFGNSFNNSSIIFILKNPVYIGKITWKKKDIRKSKDPNKVKDTRTRDKSEWII |
| | VDGKHDPIIDQITWKQAQEILNNRYHVPYKLVNGPANPLAGLIICTTCKSKMVMRKLRGTDRILCK |
| | NNKCNNISNRFDAVEKSVVESLENYLKAYKVNLPELNKTSNLKLYEQQISTLKKELKILNEQKLKLF |
| | DFLERGIYDEDTFLKRSKNLDERIEITNESLSNLNQIIAKENKAIKKEDIIKFEKVLDSYKSTADIRLKN |
| | ELMKTLIFKIEYTKNKKGNDFKIKVFPKLKPLNI |
| 360 | MIAAIYSRKSKFTGKGESVENQIEMCKEYLKRNFNNIDDIEIYEDEGFSGKDTNRPKFKKMIKAAKN |
| | KKFNILICYRLDRISRNVADFSNTIEELQKYNIDFISIKEQFDTSTPMGRAMMNIAAVFAQLERETIAE |
| | RIKDNMVELAKTGRWLGGTSPLGYKSEPIEYSNEDGKSKKMYKLTEVENEMNIVKLIYKLYLEKR |
| | GFSSVATYLCKNKYKGKNGGEFSRETARQIVINPVYCISDKTIFKWFKSKGATTYGTPDGIHGLMV |
| | YNKREGGKKDKPINEWHAVGKHRGVISSDIWLKCQNLIQQNNAKSSPRSGTGEKFLLSGMVVCKE |
| | CGSGMSSWSHFNKKTNFMERYYRCNLRNRASNRCSTKMLNAYKAEEYVANYLKELDINAIKKMY |
| | HSNKKNIIDYDAKYEVNKLNKSIEENKKIIQGIIKKIALFDDLDILGMLKNELERLKKENDEMKIKLK |
| | ELKSILELEDEEEIFLSTMEENISNFKKFYDFVNITQKRILIKGLVESIVWDTGGEEKILEINLIGSNTKL |
| | PSGKVKRRE |
| 361 | MKVAIYTRVSTLEQKEKGHSIEEQERKLRAYSDINDWTIQGVYVDAGYSGAKTDRPELNRLKENLS |
| | KIDLVLVYKLDRLTRNVKDLLDLLEIFERENVSFRSATEVYDTSTAMGRLFVTLVGAMAEWERETI |
| | RERAMMGKQAAIRKGMILTPPPFYYDRVDNKYIPNKYKDVVVWAYEEVKKGNSAKGIARKLNAS |
| | DIPPPNGIQWEDRTITRALRSPLSKGHYFWGDIFIENSHEPIITDEMYNEIKERLNERVNAKTITHTSV |
| | FRGKLICPNCNGRLCLNTSYRKLKRGDVIHKNYYCNNCKVNKSGAFSFTEKEALKVFYDYLSKLD |
| | LSKYKAKEKEDKKIVTIDINKVMEQRKRYHKLYANGMMQEEELFELIKETDEKISEYEKQKERVPK |
| | KRLDVSKIKNFKNILLDSWNAFTLEDKEDFIKMAIKSIEIEYIHVKRGKTKHSIKIKNIDFY |
| 362 | MKTAIYLRKSRADLEAEARGEGETLAKHRSTLLKIAKEMNLNVLSVREEIVSGESLVKRPEMLALL |
| | EEIEDNKYDAVLCMDMDRLGRGGMKEQGIILETFKRSNTKIMTPRKTYDLNDEWDEEYSEFEAFM |
| | ARKELKIITRRMQRGRIASVEAGNYLGTHAPFGYDIHRLNKRERTLTINSEEASVVRMIFDWYANED |
| | MGASAIRNKLNDLGYKSKLGNDWNPYSILDILKNNIYIGKVTWQKRKEVKRPDAVKRSCARQDKS |
| | DWIIADGKHEPIIPESLFEQAQEKLNSRYHVPYNTNGIKNPLAGIIKCSKCGYSMVQRYPKNRKETM |
| | DCKHRGCENKSSYTELIEKRLLEALKEWYINYKADFEAHKQGDKLKETQVIQMNEAALRKLEKEL |
| | VDVQKQKNNLHDLLERGVYTVDMFLERSQVISDRINEITSTMENLKKEIKTEIKKEKVKKDTIPQVE |
| | HVLDLYFKTDDPKKKNSLLKSVLEKAVYKKEKWQRLDDFELVLYPKLPQDGDI |
| 363 | MLRCAIYIRVSTEEQAMHGLSMDAQKADLTDYAKKHNYEIIDYYVDSGKTARKRLSKRKDLQRMI |
| | EDVKLNKIDIIIFTKLDRWFRNVRDYYKIQEVLEDHNVDWKTIFENYDTSTANGRLHINIMLSVAQD |
| | EADRTSERIKRVFENKLKNNEPTSGSLPIGYKIKEKSIIIDEEKAPIAKDVFDFYYYHQSQTKVFKEIL |
| | NKYNLSLCEKTIRRMLENKLYIGIYREHENFCPPLIDKNKFDEVQLILKRRNIKYIPTKRIFLFTSLLIC |
| | KECRHKMIGNAQIRNTKAGKIEYILYRCNQSYARHTCNHRKVIYENKIETYLLNNIESELKKFIYDY |
| | ELEDIPKVKNKVNKTNIKRKLEKLKELYINDLIDIDMYKEDYKKYTEILNTKEEKIEQRNLQPLKDF |
| | LNSDFKSLYSSISREEKRLLWRGIISEIQIDCNNDITIIPHP |
| 364 | MYRPESLDVCIYLRKSRKDVEEERRAIEEGSSYNALERHRKRLFAIAKAENHNIIDIFEEVASGESIQE |
| | RPQMQQLLRKLEGNEIDGVLVIDLDRLGRGDMLDAGMIDRAFRYSSTKIITPTDVYDPDDESWELV |
| | FGIKSLISRQELKSITKRLQNGRIDSVKEGKHIGKKPPYGYLKDENLRLYPDPEKAWIVKKIFELMCD |
| | GKGRQMIAAELDRLGIDPPVTKRGAWDSSTITSIIKNEVYTGVIVWGKFKHKKRNGKYTRHKNPQE |
| | KWIMYENAHEPIISKELFDAANEAHSSRHKPAVITSKKLTNPLAGILKCKLCGYTMLIQTRKDRPHN |
| | YLRCNNPACKGKQKQSVFNLVEEKLLYSLQQIVDEYQAQKVEEVEIDDSKLISFKEKAIISKEKELK |
| | ELQAQKGNLHDLLEQGIYTVEIFLERQKNLVERITSIENDIEVLQKEIETEQIKEHNKTEFIPALKTVIE |
| | SYHKTTNIELKNQLLKTILSTVTYYRHPDWKTNEIALIQVYFKI |
| 365 | MITTNKVAIYVRVSTTNQVEEGYSIDEQKDKLEAYCKIKDWKIYDVYVDGGFSGANTQRPELERLI |
| | SDVKRKKVDIVLVYKLDRLSRSQKDTLFLIEDVFAKNDVAFISLQENFDTSTPFGKASIGMLSVFAQ |
| | LEREQIKERMMLGKEGRAKNGKSMSWTTIAFGYDYSKETGVLSVNPTQALIVNRIFTEYLNGKPVV |
| | KIIRDLNAEGHVGRKRPWGETITKYLLKNETYLGKVKYKDKVYEGQHEPIITQELFDLVQLEVERR |
| | QISAYEKYNNPRPFRAKYMLSGLMKCGYCGASLGLRYTRKDKNGISHHKYQCRNRHSKDLEKRC |
| | ESGWYSKEELERGVIKELERIKFDPKYKNETLAKKEETIKVEEIKKQLERINNQVSKLTELYLDEIITR |
| | KELDEKNDKIKTERQFLEEQLENQKSNVLSIRKRKLTRLLKDFDVEKLSYEDASKIVKNIIKEIIVTK |
| | DGMSITLDF |
| 366 | MITTRKVAIYVRVSTTNQAEEGYSIQGQIDSLIKYCEAMGWIIYEEYTDAGFSGGKIDRPAMSKLITD |
| | AKHKRFDTILVYKLDRLSRSVRDTLYLVKDVFNQNNIHFVSLQENIDTSSAMGNLFLTLLSAIAEFE |
| | REQITERMTMGKIGRAKSGKTMAWTYTPFGYDYNKEKGELILDPAKAPIVKMIYTDYLKGMSIQKI |
| | VDKLNKMDYNGKDCTWFPHGVKHLLDNPVYYGMTRYNNKLFPGNHQPIITKELFDKTQRERQRR |
| | RLGIEENHYTIPFQAKYMLSKFLRCRQCGSRMGLELGRPRKKEGKRSKKYYCLNSRPKRTASCDTP |
| | LYDAETLEDYVLHEIAKIQKDPSIASRQKHIEDHELKYKRERIEANINKTVNQLSKLNNLYLNDLITL |
| | EDLKTQTNTLIAKKRLLENELDKTCDNDDELDRQETIADFLALPDVWTMDYEGQKYAVELLVQRV |
| | KVDRDNIDIHWTF |
| 367 | MKAIAIYARKSLFTGKGDSIGAQVDTCKRFIDYKFANEDYEIRTFKDEGWSGKTTDRPDFTNMVNL |
| | IKSKKIDYVITYKLDRIGRTARDLHNFLYELDNLGIVYLSATEPYDTTTSAGRFMISILAAMAQMERE |
| | RLAERVKSGMIQIAKKGRWLGGQCPLGFDSKREIYIDDMGKERQMMRLTPNKEEIKIVKLIYDKYL |
| | EMGSMSQVRKYCLENSIRGKNGGDFSTNTLKQLLTSPIYVKSSDNIFKYLESQNINVFGTPNGNGM |
| | LTFNKTKEIRIERDKSEWIAAVGKHKGIIDDNKWLQIQQQLQQQSEKQIKSSGRQGTTSTGLLSGIIK |
| | CSKCGNNLLIKTGHKSKKNPGTTYSYYVCGKKDNSYGHKCDNKNVRTDEADSAVITQLKLYNKEL |
| | LIKNLKEALIQNEKTDTDNIEILESKLKEKEKAVSNLVKKLSLIDDESISNIILNEVTNINKEINDIKLQ |
| | LSNETLKINEVTKATLDTEIYIKILENFNKKIDDITDPIEKMNLLKSALESVEWNGDSGEFKINLIGSK |
| | KK |
| 368 | MKVAIYVRVSTDEQAKEGFSIPAQRERLRAFCASQGWEIVQEYIEEGWSAKDLDRPQMQRLLKDIK |
| | KGNIDIVLVYRLDRLTRSVLDLYLLLQTFEKYNVAFRSATEVYDTSTAMGRLFITLVAALAQWERE |
| | NLAERVKFGIEQMIDEGKKPGGHSPYGYKFDKDFNCTIIEEEADVVRMIYRMYCDGYGYRSIADRL |
| | NELMVKPRIAKEWNHNSVRDILTNDIYIGTYRWGDKVVPNNHPPIISETLFKKAQKEKEKRGVDRK |
| | RVGKFLFTGLLQCGNCGGHKMQGHFDKREQKTYYRCTKCHRITNEKNILEPLLDEIQLLITSKEYF |
| | MSKFSDRYDQQEVVDVSALTKELEKIKRQKEKWYDLYMDDRNPIPKEELFAKINELNKKEEEIYSK |
| | LSEVEEDKEPVEEKYNRLSKMIDFKQQFEQANDFTKKELLFSIFEKIVIYREKGKLKKITLDYTLK |
| 369 | METMPQPLRALVGARVSVVQGPQKVSQQAQLETARKWAEAQGHEIVGTFEDLGVSASVRPDERP |
| | DLGKWLTDEGASKWDVIVWSKMDRAFRSTKHCVDFAQWAEERQKVVMFAEDNLRLDYRPGAA |
| | KGIDAMMAELFVYLGSFFAQLELNRFKSRAQDSHRVLRQTDRWASGLPPLGYKTVPHPSGKGFGL |
| | DTDEDTKAVLYDMAGKLLDGWSLIGIAKDLNDRGVLGSRSRARLAKGKPIDQAPWNVSTVKDAL |
| | TNLKTQGIKMTGKGKHAKPVLDDKGEQIVLAPPTFDWDTWKQIQDAVALREQAPRSRVHTKNPM |
| | LGIGICGKCGATLAQQHSRKKSDKSVVYRYYRCSRTPVNCDGVFIVADEADTLLEEAFLYEWADQ |
| | PVTRRVFVPGEDHTYELEQINETIARLRRESDAGLIVSDEDERIYLERMRSLITRRTKLEAMPRRSAG |
| | WVEETTGQTYGEAWETEDHQQLLKDAKVKFILYSNKPRNIEVVVPQDRVAVDLAI |
| 370 | MRNKVAIYVRVSTASQADEGYSIDEQKSKLEAYCEIKDWKIYDTYIDGGFSGANTQRPELERLISDA |
| | KRKKIDIVLVYKLDRLSRSQKDTLFLIEDVFAKNDVAFISLQENFDTSTPFGKASIGMLSVFAQLERE |
| | QIKERMMLGKEGRAKNGKSMSWTTIPFGYDYSKETGILSVNPTQALIVKRIFTEYLNGKSVVKIIRD |
| | LNAEGHVGRKRPWGETITKYLLKNETYLGKSKYKGKVFEGQHDAIISQELFDLVQLEVEKRQISAF |
| | EKYNNPRPFRAKYMLSGLMKCGYCGASLGLYVAPKNKNGVSKYKYQCRHRYHKDKAIRCNSGW |
| | YSKDELEKRVIKELERLKFDPKYKKETLAKKDETIKVEDIKKQLERINKQVSKLTELYLDEVITRKD |
| | LDEKNAKIKTERQYLEEQLENQKSNVMSIRKRKLSRLLKDFDIEKLSYEEASKIVKSVIKEIVVTKDD |
| | MTITLDF |
| 371 | MKVAIYTRVSTLEQREKGHSIDEQERKLRSFCDINDWTVKDVYVDAGFSGAKRDRPELTRLLDDIS |
| | EFDLVLVYKLDRLTRSVRDLLDLLEVFENNNVAFRSATEVYDTTTAIGRLFVTLVGAMAEWERETI |
| | RERSLMGKRAAIKKGMILTAPPFYYDRVNNTYIPNQYKDVVLDVYNKVKKGYSIAHIARLYNNSD |
| | VKPPNGNEEWTTRMLMHALRNPVTRGHYQWGEIYIEDSHEPIITDEMYNTIIDRLDKHTNTKVVAH |
| | TSVFRGKLICPNCGYALTLNSQKRKRKNDTIVYKTYYCNNCKITKGMKPHHITETETLRVFKDHLS |
| | KIDLKQYETQEKEKQSHVTIDLSKVMEQRKRYHKLYASGMMQENELFELIKETDEMIEEYEKQRK |
| | QVDVKEFDICKIKEIKDVLLKSWDIFTLEDKADFIQMSIKAINIEYTKLKRGKSSNSMKIKDIEFY |
| 372 | MITTNKVAIYVRVSTTNQVEEGYSIDEQKDKLSSYCDIKDWNVYKVYTDGGFSGSNTDRPALESLI |
| | KDAKKRKFDTVLVYKLDRLSRSQKDTLHLIEDVFIKNGIEFLSLQENFDTSTPFGKAMIGLLSVFAQ |
| | LEREQIKERMQLGKLGRAKSGKSMMWAKTSYGYDYHKETGTVTINPAQALTIKFIFESYLRGRSIT |
| | KLRDDLNEKYPKHVPWSYRAVRTILDNPVYCGFNQYKGEIYPGNHEPIISKEEYDKTQSELKIRQRT |
| | AAENVNPRPFQAKYILSGIAQCGYCGAPLKIMLGVKRKDGSRLKKYECHQRHPRTLRGVTTYNDN |
| | KKCDSGFYYKDKLEAYVLKEISKLQDDADYLDKIFSGDNAETIDRESYKKQIEELSKKLSRLNDLYI |
| | DDRITLEELQSKSAEFISMRGTLETELENDPALRKNKRKADMRKLLNAEKVFSMDYESQKVLVRRL |
| | INKVKVTAEDIVINWKI |
| 373 | MKLRAAIYVRVSTMEQAEEGYSISAQTEKLKSYANAKDYQVVKVFTDPGYSGAKLERPGLQNMIK |
| | SIESKEIDVVLVYKLDRLSRSQKNTLFLIEDVFLKNHVQFTSMQESFDTSTSFGRAMIGILSVFAQLE |
| | RDAITERMQMGAKERAKAGMWRGGPQSRLPFGYRYIDGVLLVDDYEAMIVKYMYTEFIKGTPLT |
| | KIQSKVAAKFPVKETLIYPSIMKNILQNNIYIGKIKYAGETYEGLHEHILDTETYDKAQQLWEHRNT |
| | NKKKYILSKYLLSGILYCGHCGGKMASTGAGLLKSGERVTDYICYSKKGTPSHMVVDRNCPSKRH |
| | RVNRLDPKIVELLKTITFEEMQKDNSFTDNTTTIKSEIESLDTKISKLLDLYQDGLVPIDVLNDRISKL |
| | NDDKELLQETLISQKKQIHPEEIAKNIQTAKDFDWANSDSAAKRAMVRALINKVELTNEDMKIEWN |
| | I |
| 374 | MKVATYVRVSTDEQAKEGFSIPAQRERLRAFCESQGWEIVEEYIEEGWSAKDLDRPQMQRLLKDIK |
| | KGNIDIVLVYRLDRLTRSVLDLYLLLQTFEKYNVAFRSATEVYDTSTAMGRLFITLVAALAQWERE |
| | NLAERVKFGIEQMIDEGKKPGGHSPYGYKFDKDFNCTIIEDEANTVRMIYRMYCDGYGYHSIAKRL |
| | NELGIKPRIAKEWNHNSVRDILTNDIYIGTYRWGNKVVLNNHPPIISETLFRKVQKEKEKRRVDRTR |
| | VGKFLLTGLLYCGNCNGHKMQGTFDKREQKTYYRCLKCNRITNEKNILEPLLDEIQLLITSKEYFMS |
| | KFSDQYDQKEEVDVSALKKELEKIKRQKEKWYDLYMDDRNPIPKEDLFAKINELNKKEEEIYNKL |
| | NEVEPEDKEPVEEKYNRLSKMIDFKQQFEQANDFTKKELLFSIFEKIVIYREKGKLKKITLDYTLK |
| 375 | MKYLALHENSRIAVYSRKSREDRDSEDTLAKHRNELEYLIKRENFKNVQWFEKVVSGETIDERPMF |
| | SLLLPRIENGEFDAVCAVAMDRLSRGSQIDSGRILEAFKQSGTLFITPKKTYDLSIEGDEMLSEFESII |
| | ARSEYRAIKRRTINGKKNATREGRLHSGSVPYGYKWDKNLKAAVVVEEKKKIYRMMIKWFLEEE |
| | YSCTVIAEMLNELKVPSPSGRSIWYGEVVSEILSNDFHRGYVWFGKYKKSKSNNSIVQNKNLDEVLI |
| | AKGHHETMKTDEEHALILNRIEKLRTYKVAGRRLNMNTHRLSGIVRCPYCHKAQAIEQPKGRRKH |
| | VRKCLRKSAERTKECEETKGIHEEVLFQSIMKEIKKYNESLFSPTEQDVNDDSYTAQLIGLREKAVK |
| | KAKGRIERIKEMYLDGDISKTEYKEKLKISQETLQKAENELAELIASTEFQNALSAETKKEKWSHHK |
| | VQEMIESTDGMSNSEINLILKMLISHVTYTVEDLGDGTKNLNIKVYYN |
| 376 | MKITLLYYIKKFNIYCNRYLSQQINISVDIIGFYQFKNVTNSVTDVLKRGDNLDRICIYLRKSRADEE |
| | LEKTIGVGETLSKHRKALLKFAKEKKLNIMEIKEEIVSADSIFFRPKMIELLKEVENNQYTGVLVMDI |
| | QRLGRGDTEDQGIIARIFKESHTKIITPMKTYDLDDDLDEDYILFESFMGRKEYKMIKKRMQGGRV |
| | RSVEDGNYIATNPPFGYDIHWINKSRTLKFNSKESEIVKLIFKLYTEGNGAGTISNYLNSLGYKTKFG |
| | NNFSNSSIIFILKNPVYIGKITWKKKDIRKSKDPHKVKDTRTRDKSEWIIADGKHEPIIDEKIWNKAQE |
| | ILNNKYHIPYKIANGPANPLAGVVICSKCNSKMVMRKYGKKLPHLICNNKECNNKSARFDYIEKAV |
| | LEGLDEYLKNYKVNVKANNKTSDIEPYEQQSNALNKELILLNEQKLKLFDFLEREIYTEEIFLERSK |
| | NLDERINTTTLAINKIKKILDNEKKKNNKNDIVKFEKILEGYKKTNDIQKKNELMKSLVFKIEYKKE |
| | QHQRNDGLLYIYFLSFCVRCISYLTQFISFFVYPYRILEIYLTFSFFIISYEH |
| 377 | MKVAIYTRVSSAEQANEGYSIHEQKKKLISYCEIHDWNEYKVFTDAGISGGSMKRPALQKLMKHL |
| | SSFDLVLVYKLDRLTRNVRDLLDMLEEFEQYNVSFKSATEVFDTTSAIGKLFITMVGAMAEWERET |
| | IRERSLFGSRAAVREGNYIREAPFCYDNIEGKLHPNEYAKVIDLIVSMFKKGISANEIARRLNSSKVH |
| | VPNKKSWNRNSLIRLMRSPVLRGHTKYGDMLIENTHEPVLSEHDYNAINNAISSKTHKSKVKHHAI |
| | FRGALVCPQCNRRLHLYAGTVKDRKGYKYDVRRYKCETCSKNKDVKNVSFNESEVENKFVNLLK |
| | SYELNKFHIRKVEPVKKIEYDIDKINKQKINYTRSWSLGYIEDDEYFELMEEINATKKMIEEQTTENK |
| | QSVSKEQIQSINNFILKGWEELTIKDKEELILSTVDKIEFNFIPKDKKHKTNTLDINNIHFKF |
| 378 | MSKKVAIYTRVSTTNQAEEGYSIDEQIDKLKMYCEAMDWKVSEIYTDAGFTGSKLTRPAMEKMIT |
| | DIGLKKFDTVIVYKLDRLSRSVRDTLYLVKDVFTKNEIDFISLSESIDTSSAMGSLFLTILSAINEFERE |
| | NIKERMTMGKIGRAKSGKSMMWAKTAFGYSHNQETGILEINPLEASIVEQIFNEYLKGTSITKLRDK |
| | LNEDGHIAKELPWSYRTIRQTLDNPVYCGYIKYKNNTFEGLHKPIISHETYLSVQKELEARQQQTYE |
| | KNNNPRPFQAKYLLSGIARCGYCGAPLRIVLGHRRKDGSRTMKYQCVNRFPRKTKGVTTYNDNKK |
| | CDSGAYDMQWIEDIVLKTLNGFQKSDKKLRKILNIKEESKVDTSGFQKQLKSINNKIQKNSDLYLN |
| | DFITMDDLKKRTEMLQGEKKLIQARINEVDKPSTSEIFDLVKSELGETTISKISYEDKKKIVNNLISKV |
| | DVTADNIDIIFKFQLA |
| 379 | MRTVRRIQPIKSPCKPRFKVAAYARVSDSRLHHSLSTQISYYNRLIQAHPDWELVGIYYDEGISGKE |
| | QSNRQGFLNLIKDCEDGKIDRIITKSIARFGRNTVELLTTVRQLRLKNIGVTFEKENIDSLSSEGELML |
| | TLLASVAQEESQNLSENIRWRIQKKFEKGIPHTPQDMYGYRWDGEQYQIEPNEAKVIRKVFKWYLD |
| | GDSVQQIVDKLNQEQVLTRLGNPFTVASIREFFKQEAYFGRLVLQKTYREAFSRNPKRNKGQRNKY |
| | IIENAHEPIVTKEYFDLVLHEKERRNQLMHQESHLNKGIFRDKISCSECGCLMIVKVDSKQVNKTVR |
| | YYCRTRNRFGASSCSCRTLGEKRLLASFKSKLGIVPDKEWVENNIKHIEYDFGYRILRVTPVKGRKY |
| | LIEIREGRY |
| 380 | MKGESELDKKAAIYIRVSTQEQATEGYSIQAQTDRLIKYVEAKDFILYKKYIDAGYSASKLERPAM |
| | QDLIQDVQSKKVDVVIVYKLDRLSRSQKDTMYLIEDIFRPNDVELISMQESFDTSTAFGSATVGMLS |
| | VFAQLERKSISERMITGRVERAKKGFYHTGGQDRPPAGYQFNSDNQLIINEYEAAAIKDLFRLYND |
| | GLGKSSISEYLKKNYPGKNKWLPSSIDRMLKNSLYIGKVKFSGAEYDGIHEPIIDEVTFYKTQKEIAR |
| | RKQTNTKRYNYVALLGGLCECGICGAKMANRRAVGRKGKVYRYYRCYSKKGSPKHMMKTDGCS |
| | SKAQQQFIIDEAVINNLKNIDVEAELKRRSAPQTNTSLISSQIESIDKQINKLIDLFQVDSMPLDVISEK |
| | IDKLNKEKQSMEKLLERKNKLDKTELQHRFDVLKSFDWDNSSIESKRVVIEMLVQKVIIHDNSIEIIL |
| | VE |
| 381 | MKRDLPSTFRGSRTPGEPWLGYIRVSTWREEKISPELQQSAIESWAARTGRRIVDWIVDLDATGRNF |
| | KRKIMGGIQRVEGREAVGIAVWKFSRFGRNDLGIAINLARLEQAGGDLASATEEVDARTAVGRFNR |
| | AILFDLAVFESDRAGEQWKETHAHRRALKLPATGRQRFGYVWHPRRVPDLTAPGGFRLQEERYER |
| | HPEFAPVAAELYERKLAGQGFSQLAYWLNDELLIPTTRGNRWGTNTVQRYLDSGFAAGLLRVHDP |
| | ECRCKLGQDHFSACKENRWLWLPGAQPALIVPEQWKEYGAHREQTRKTPPRARRASYPTSGIMRH |
| | GHCRGTAVARSGRDGKGGFVPGHVFVCFNRRNKGKSACEPGLYVRRDEVEAEVLKWLADTVAD |
| | DIDNAPALPAQRTAPGTAPDPRARLVEERTRTEAELAKIEGALDRLVTDYALDPDKYPADTFGRVR |
| | DQLLGKKGDIIKHLKSLSEVEVAPTREEFRPLIVGLLQEWDILHTTEKNAILRRLLRRLVIHNRKSDQ |
| | GAQWSVVRSFEFHPVWEPDPWS |
| 382 | MKRDLPSTFRGSRTPGEPWLGYIRVSTWREEKISPELQQSAIESWAARTGRRIVDWIVDLDATGRNF |
| | KRKIMGGIQRVEGREAVGIAVWKFSRFGRNDLGIAINLARLEQAGGDLASATEEVDARTAVGRFNR |
| | AILFDLAVFESDRAGEQWKETHAHRRALKLPATGRQRFGYVWHPRRVPDLTAPGGFRLQEERYER |
| | HPEFAPVAAELYERKLAGQGFSQLAYWLNDELLIPTTRGNRWGTNTVQRYLDSGFAAGLLRVHDP |
| | ECRCKLGQDHFSACKENRWLWLPGAQPALIVPEQWKEYGAHREQTRKTPPRARRASYPTSGIMRH |
| | GHCRGTAVARSGRDGKGGFVPGHVFVCFNRRNKGKSACEPGLYVRRDEVEAEVLKWLADTVAD |
| | DIDNAPALPAQRTAPGTAPDPRARLVEERTRTEAELAKIEGALDRLVTDYALDPDKYPADTFGRVR |
| | DQLLGKKGDIIKHLKSLSEVEVAPTREEFRPLIVGLLQEWDILHTTEKNAILRRLLRRLVIHNRKSDQ |
| | GAQWSVVRSFEFHPVWEPDPWS |
| 383 | MSVKVEGMVILAGGYDRQSAERENSSTASPATQRAANRGKAEALAKEYARDGVEVKWLGHFSEA |
| | PGTSAFTGVDRPEFNRILDMCRNREMNMIIVHYISRLSREEPLDIIPVVTELLRLGVTIVSVNEGTFRP |
| | GEMMDLIHLIMRLQASHDESKNKSVAVSNAKELAKRLGGHTGSTPYGFDTVEEMVPNPEDGGKL |
| | VAIRRLVPSAHTWEGAHGSEGAVIRWAWQEIKTHRDTPFKGGGAGSFHPGSLNGLCERLYRDKVP |
| | TRGTLVGKKRAGSDWDPGVLKRVLSDPRIAGYQADIAYKVRADGSRGGFSHYKIRRDPVTMEPLT |
| | LPGFEPYIPPAEWWELQEWLQGRGRGKGQYRGQSLLSAMDVLYCYGSGQLDPETGYSNGSTMAG |
| | NVREGDQAHKSSYACKCPRRVHDGSSCSITMHNLDPYIVGAIFARITAFDPADPDDLEGDTAALMY |
| | EAARRWGATHERPELKGQRSELMAQRADAVKALEELYEDKRNGGYRSAMGRRAFLEEEAALTLR |
| | MEGAEERLRQLDAADSPVLPIGEWLGDRGSDPTGPGSWWALAPLEDRRAFVRLFVDRIEVIKLPKG |
| | VQRPGRVPPIADRVRIHWAKPKVEEETEPETLNGFTAAA |
| 384 | MSARDYDIEAEWTPADLALLKELEEAEALLPADAPRALLSVRLSVFTDDTTSPVRQELDLRQLARE |
| | KGYRVVGLASDLNVSATKVPPWKRKSLGDWLNNRAPEFDALLFWKIDRFIRNLNDLNVMIRWSET |
| | YSKNLISKNDPIDLTTTMGKMMVSLLGGVAEIEAANTKTRVESLWDYTKTQGEWHVGKPPFGYKT |
| | GRDAAGKVVLVEDPPAVETLHTARELVMSGMSTTAAAKELKERGLISSTTATLTRRLRNPGILGLR |
| | VEEDKDGGIRRSKLILGRDGQPIRIADPIFTEEQFEELQAVLDKRGKRQPHRQPGGATSFLGVLKCA |
| | ECGTNMINHFTRNRHGDYAYLRCQGCKSGGCGAPNPQEVYDRLVEQVLAVLGDFPVEMREYARG |
| | EEKRKELKRLEESIAYYMKELEPGGRFTKTRFTQDQAEGTLDKLIAELEAIDPESAKDRWVYVAGG |
| | KTFREHWEEGGIDAMSADLIRAGIRCQVTRTKVPKVRAPQVHLKLMIPKDVRTRLVIRPDDFGQTF |
| 385 | MSARDYDIEAEWTPADLALLKELEEAEALLPADAPRALLSVRLSVFTDDTTSPVRQELDLRQLARE |
| | KGHRVVGLASDLNVSATKVPPWKRKSLGDWLNNRAPEFDALLFWKIDRFIRNLNDLNVMIRWSET |
| | YSKNLISKNDPIDLTTTMGKMMVSLLGGVAEIEAANTKTRVESLWDYTKTQGEWHVGKPPFGYRT |
| | GRDDSGKVVLVEDPLAVETLHTARELVMTGMSTTAAAKELKERGLISSTTATLTRRLRNPGILGLR |
| | VEEDKDGGIRRSKLILGRDGQPIRIADPIFTEEQFEELQAVLDKRGKRQPHRQPGGATSFLGVLKCA |
| | ECGTNMINHFTRNRHGDYAYLRCQGCKSGGYGAPNPQEVYDRLVEQVLAVLGDFPVEMREYARG |
| | EEKRKELKRLEESIAYYMKELEPGGRFTKTRFTQDQAEGTLDKLIAELEAIDPESAKDRWVYVAGG |
| | KTFREHWEEGGIDAMSADLIRAGIRCQVTRTKVPKVRAPQVHLKLMIPKDVRTRLVIRPDDFGQTF |
| 386 | MWACSHLRADGTTPTSSSTLLTMSARDYDIEAEWTPADLALLKELEEAEALLPADAPRALLSVRLS |
| | VFTDDTTSPVRQELDLRQLAREKGHRVVGLASDLNVSATKVPPWKRKSLGDWLNNRAPEFDALLF |
| | WKIDRFIRNLNDLNVMIRWSETYSKNLISKNDPIDLTTTMGKMMVSLLGGVAEIEAANTKTRVESL |
| | WDYTKTQGEWHVGKPPFGYKTARDEAGKVVLIEDPLAVETLHTARELVMSGMSTTAAAKVLKER |
| | GLISSTTATLTRRLRNPGVLGLRVEEDKDGGIRRSKLILGRDGQPIRIADPIFTEEQFEELQAVLDKRG |
| | KRQPHRQPGGATSFLGVLKCAECGTNMINHFTRNRHGDYAYLRCQGCKSGGYGAPNPQEVYDRL |
| | VEQVLTVLGDFPVEMREYARGEEKRKELKRLEESIAYYMKELEPGGRFTKTRFTQDQAEGTLDKLI |
| | AELEAIDPESAKDRWVYVAGGKTFREHWEEGGIDAMSADLIRAGIMCQVTRTKVPKVRAPQVHLK |
| | LMIPKDVRTRLVIRPDDFGQTF |
| 387 | MSDRASTYDIEAEWSPADLALLRSLEEAETLLPPDAPRALLSVRLSVFTEDTTSPVRQELDLRQLAR |
| | DKGMRVVGVASDLNVSATKVPPWKRKELGDWLGNKTPQFDALLFWKIDRFIRNMGDLSRMIEW |
| | ANRYEKNLISKNDPIDLKTPIGKMMTTLLGGVAEIESANTKARVESLWDYAKTQSDWLVGKPAYG |
| | YVTQRDESGKVSLAVDPKAREALHLARELVLGGMAARSVAEELKKREMVTPGLTAATLLRRMRN |
| | PALMGYRVEEDKRGGLRRSKLVLGHDGKPIRVADPVFTEEEFETLQAVLDSRGKNQPPRQPSGAT |
| | KFLGVLKCVDCRSNMIVHFTRNKHGEYAYLRCQKCKSGGLGAPHPQEVYDALVEQVLAVLGDFP |
| | VERREYARGEEARAEVKRLEESIAYYMQGLEPGGRYTKTRFTRENAERALDKLIAELEAVDPETTE |
| | DRWIYEPIGKTFRQHWEEGGMEAMALDLIRAGITCDVTRTKVPRVRAPQVELDLDIPSDVRERLVM |
| | RRDDFAEAF |
| 388 | MSKRAVIYTRVSRDDTGEGQSNQRQEAECRRLTDYRRLDVVAVEADISISASKGLERPAWLRVLG |
| | MIERGEVDYVIAYHMDRVTRSMTELEQLIEMCLKYDVGVATVSGDIDLTTDVGRMVARIIGAVAR |
| | AEVERKSARQKLANAQRAAEGKPHVSGIRPFGYADDHRQVVTIEAQAIRAAAEAALAGESMIGIAE |
| | SWSKDGLLSARARRGHDKGNRPTKAAWSARGVRNVLVNPRYAGIRLYNGERVGQGDWEPILDVE |
| | THLRLVEKLTDPTRRKGTVKTGRVAASLLTAIARCEVCGQTVRASSVRGRQTYACRNSHAHVDRS |
| | TADLMTQEWVISRLADPDTLAKLAPSGDDRVDEAKATIEKRREALKTYARLLATGAMDEDQFTEA |
| | SAVARSEMQEAEAVLTEAGTGDLLAGLDVGSDAVGPQFLALSLARQRGIVEALVDVTLRPASKAR |
| | KVVTPEHERVVLADR |
| 389 | MRVLGRIRLSRMMEESTSVERQREFIETWARQNDHEIVGWAEDLDVSGSVDPFDTQGLGPWLKEP |
| | KLREWDILCAWKLDRLARRAVPLHKLFGMCQDEQKVLVCVSDNIDLSTWVGRLVASVIAGVAEG |
| | ELEAIRERTLSSQRKLRELGRWAGGKPAYGFKAQEREDSAGYELVHDEHAANVMLGVIEKVLAGQ |
| | STESVARELNEAGELAPSDYIRARAGRKTRGTKWSNAQIRQLLKSKTLLGHVTHNGATVRDDDGIP |
| | IRKGPALISEEKFDQLQAALDARSFKVTNRSAKASPLLGVAICGLCGRPMHIRQHRRNGNLYRYYR |
| | CDSGSHSGGGGAAPEHPSNIIKADDLEALVEEHFLDEVGRFNVQEKVYVPASDHRAELDEAVRAV |
| | EELTQLLGTMTSATMKSRLMGQLTALDERIARLENLPSEEARWDYRATDQTYAEAWEEADTEGRR |
| | QLLIRSGITAEVKVTGGDRGVRGVLEFHLKVPEDVRERLSA |
| 390 | MRVLGRIRLSRVMEESTSVERQREIIETWARQNDHEIIGWAEDLDVSGSVDPIALTPALGPWLTDHR |
| | KHEWDILVAWKLDRLSRRAIPMNKLFGWVMENDKTLVCVSENLDLSTWIGRMIANVIAGVAEGE |
| | LEAIRERTKGSQKKLRELGRWGGGKPYYGYRAQEREDAAGWELVPDEHASAVLLSIIEKVLEGQS |
| | TESIARELNERGELSPSDYLRHRAGKPTRGGKWSNAHIRQQLRSKTLLGYSTHNGETIRDERGIAVR |
| | KGPALVSQDVFDRLQAALDSRSFKVTNRSAKASPLLGVLICRVCERPMHLRQHHNKKRGKTYRYY |
| | QCVGGVEKTHPANLTNADQMEQLVEESFLAELGDRKIQERVYIPAESHRAELDEAVRAVEEITPLL |
| | GTVTSDTMRKRLLDQLSALDARISELEKLPESEARWEYREGDETYAEAWNRGDAEARRQLLLKSG |
| | ITAAAEMKGREARVNPGVLHFDLRIPEDILERMSA |
| 391 | MRVLGRLRLSRSTEESTSIERQREIVTAWAESNGHTLVGWAEDVDVSGAIDPFDTPSLGPWLDERR |
| | GEWDILCAWKLDRLGRDAIRLNKLFGWCQEHGKTVASCSEGIDLSTPVGRLIANVIAFLAEGEREAI |
| | RERVTSSKQKLREVGRWGGGKPPFGYMGIPNPDGQGHILVVDPVAKPVVRRIVDDILDGKPLTRLC |
| | TELTEERYLTPAEYYATLKAGAPRQKAEPDETPAKWRPTALRNLLRSKALRGYAHHKGQTVRDLK |
| | GQPVRLAEPLVDADEWELLQETLDRVQANWSGRRVEGVSPLSGVVVCITCDRPLHHDRYLVKRPY |
| | GDYPYRYYRCRDRHGKNLPAEMVETLMEESFLARVGDYPVRERVWVQGDTNWADLKEAVAAY |
| | DELVQAAGRAKSATAKERLQRQLDALDERIAELESAPATEAHWEYRPTGGTYRDAWETADTDER |
| | REILRRSGIVLAVGVDGVDGRRSKHNPGALHFDFRVPEELTQRLGVS |
| 392 | MRTNEHNFHNIEEEIKHVAVYLRLSRGEDESELDNHKTRLLNRCELNNWSYELYKEIGSGSTIDDRP |
| | VMQKLLTDVEKNLYDAVLVVDLDRLSRGNGTDNDRILYSMKVSETLIVVESPYQVLDANNESDEE |
| | IILFKGFFARFEFKQINKRMREGKKLAQSRGQWVNSVTPYGYIVNKTTKKLTPSEEEAKVVIMIKDF |
| | FFEGKSTSDIAWELNKRKIKPRRATEWRSSSIANILQNEVYVGNIVYNKSVGNKKPSKSKTRVTTPY |
| | RRLPEEEWRRVYNAHQPLYSKEEFDRIKQYFECNVKSHKGSEVRTYALTGLCKTPDGKTMRVTQG |
| | KKGTDDDLYLFPKKNKHGDSSIYKGISYNVVYETLKEVILQVKDYLDSVLDQNENKDLVEELKEEL |
| | MKKEDELETIQKAKNRIVQGFLIGLYDEQDSIELKVEKEKEIDEKEKEIEAIKMKIDNAKTVNNSIKK |
| | TKIERLLSDVQSAESEKEINRFYKTLIKEIIVDRTDENEAKIKVNFL |
| 393 | MTNPASRPKAYSYIRMSSAIQIKGDSFRRQAEASAKYAAEHDLDLIDDYKLADLGVSAFKSDNLTT |
| | GALGRFVAECEAGEIEAGSFLLIESLDRLSRDKILDAFSLFARILKTGVKIVTLSDGQVYDGSSDQVG |
| | SIYYAISVMIRSNDESKIKSTRGLANWSQKRKLAAEHGVKMSSQCPAWLKLSVDRKSYLIDKERAK |
| | IVQRIFEASASGKGANLITKELNRDKVPTFGRGALWAEAFVSKTLRNRAVLGEFQPGQYVSGKRQP |
| | AGDPIPGYFPPVIEEELFDIVQASLRGRLLAGGRRGEGQSNIFTHVAFCGYCGSKMRHRSKGSRVKG |
| | NPPHRYLTCFNRFNGPGCDCKPLPYAAFERSFLTFVRDVDLRGLLEGAKRKSEAKTIADRITVNEEK |
| | VRKADERIRDYLIKIEGAPDLAEIFMERIRELKAEKDDLVRSIEESNDALSKIKSDNVTDEELASLIST |
| | FQNPCGENRIRLADRIKSIIERIDVYPNGEIRKDDPAIDLVRASGDPDAEKIIAAMNAGSRLKDDPYFI |
| | VTFRNGAVQTVVPNPSNPDDIRVSVYAGEKTRRVEGSAYEYESD |
| 394 | MDPQHKPTRALIVIRLSRLTDETTSPERQLEACERFCAARGWEVVGVAEDLDVSAGTTSPFERPSLS |
| | QWIGDGKDNPGRIGEFDTVVFYRVDRLVRRVRHLHDVIAWSERFDVNMVSATESHFDLSTTIGALI |
| | AQLVASFAEMELEGISQRATSAHRHNVQLGKFVGGSPPFGYMPEETPDGWRLVHDPDVVPIILEVV |
| | DRVLEGEPLRRITDDLNARGATTARDLVKQRKGKETEGHKWHSNVLKRRLMSPAMLGYALRREP |
| | LTDSKGKPKLSAKGAKLYGPEEIVRGPDGLPVQRAEPILPKPLFDRVVAELEARELQKEPTKRINSM |
| | LLRVLYCGVCGQPVYRAKGQGGRSDRYRCRSIQDGANCGNPSVLTYELDDLVEESILVLMGDSER |
| | LAHVWNPGEDNASELAEVEARLADRTGLIGVGAYKAGTPQRATLDTLIEADAKLYERLKAATPRP |
| | AGWTWEPTGETFAEWWAALDTGARNVYLRNMGVRVTYDKRPVPEQVSAGEKPRVHLELGEVRK |
| | MAEQVAVTGTIGTLTRNYTRLGEIGITHVDIDAGSGKAVFVTKSGERFELPLNIPEE |
| 395 | MNYERSYLRSCQVSTLEQKEHGYSIEEQERKLKSFCEINDWSVSDVFIDAGFSGAKRDRPELQRMM |
| | NDIKRFDLVLVYKLDRLTRNVRDLLDLLEVFEQNNVAFRSATEVYDTSTAMGRLFVTLVGAMAE |
| | WERETIRERVMMGKRAAIKQGMILTPPPFYYDRVDNTYIPNDYKKVVLWAYDEVMKGNSSKAIA |
| | RKLNDSDIPPPNGKRWEDRTITRALRNPITRGHYTWGDVFIENSHEPIITEEMYQQIKERLEERINTKI |
| | VSHVSVFRGKFICPRCGGTLTMNTATRKRKKGYVTYKTYYCNTCKTKKQSFGFSENEALRVFRDY |
| | LSKLDLDKYEVKTKQKDDVVTIDIDKIMEQRKRYHKLYAKGLMQEEELFELIKETDETIAEYEKQK |
| | ELVPRKSLDIDKIKKFKNALLESWKIFSLEDKADFIKMAIKSIDIDYVKLKNRHSIKINDIEFY |

TABLE 6

| SEQ | | SEQ | |
|---|---|---|---|
| ID | | ID | |
| NO: | attL | NO: | attR |
| 396 | TCTAACTCACGACACGTTGTACTCTTACC | 727 | CAGTTTTTATTTTATGCCTTAATTATACA |
| | AACCGCACTTGCGGTATGTCAATATGGCA | | CCGCACTTGCTCCCTCAAACGCTATAATC |
| | AAAAGCTATTC | | CCCATAGTT |
| 397 | CATTTTTACCTTGCTCTTCTCTCGAATTT | 728 | AGTTTTATTTTTGTCTGTATAGGCTGTCC |
| | CAGCATCTGCGGTATGCTTATAGGGACAA | | GCATCTGCATGGCGCATAACATATTTATG |
| | AAATTATAAA | | CGCTACAG |
| 398 | ACAATCAACAAAGATGTATGGTGGTACAT | 729 | TAACATATGTACGGAAGTATAGACACTCG |
| | GCATTAATATTTAATGTGTATACTTCCGT | | ATTAATATCGGATGTATACCGACTAAAAC |
| | ATTTTTATTT | | ATTAATTC |
| 399 | TACAGACTTACATGGGACCATTCTATAGC | 730 | TCAACTTTTAACCCTGTTTTAAGACCCAG |
| | AGCTTTAAAATACTTAGCAATAAAACAGG | | TATTAAGATGCGTGAGGGACAAGATTACC |
| | GGAATTGATA | | AGACTCAG |
| 400 | TGTAATTTCGGACACGAGTTCGACTCTCG | 731 | TTGTATATTGCTAACAAAAGTTTAGCCTC |
| | TCATCTCCACCATTTCTATCAATATACAT | | ATCTCCACCAAAATATCAATATCCAAGTC |
| | AGGAAATAGT | | TTTGAATT |
| 401 | ATATGTTCCCGCAAACAGCACACGTTGAG | 732 | TATCCCCTCCTCTCAAAACATGTAGAGAC |
| | ACGGTAGTATTGATGTCAAGGGTTGATAA | | TGTAGTACTTTTGCAGTTAAAAGATAAAT |
| | GTAAGCGTGT | | AAAGGACT |
| 402 | TCGGCTTAGTGATGCCGAGTTCAGCTGGT | 733 | TTTGCAATTGCTGGTGGTTCTGGTGCTTG |
| | AAACCTTGGGCGATTGCGAGGTTTAAGGC | | GCCTTGGGTACTTGCTTCTCAGCTACTTT |
| | TTTCCACTTTT | | CCCTCTTTT |
| 403 | GTCTTCTGGACCATGATGCGCCACTTCTG | 734 | TGTATCTTGATGTACAACATTGCTCTTTA |
| | AAATTTCAAATACAGATTAATGTTGTATA | | TTTTCAAAAAGATCAGTGGTCAAACGGCT |
| | AAGTAGCCCTG | | CATTAATTT |
| 404 | CGGGCAAATTGCTGCCATATGGACCGGAG | 735 | CTATTTATTAGATGTCTAAACAGTGCATT |
| | GCGGGACTCTACAACCTATATTAGACATC | | ACTACTTTAATTCCTTGGGCGCTTATTCC |
| | TTATAAAAAGT | | TGCCGCTGC |
| 405 | TGATTTGATTGTATTGGATATTATGTTAC | 736 | AATATAGTTGTATAAAAAGTCCTTTGCCA |
| | CAGATGGCGAAGGACTTTTTGTACAACAA | | GATGGCGAAGGTTATGATATTTGTAAAGA |
| | AAAGTCACAA | | AATAAGAA |
| 406 | GCCCGTGGATTTGTTTCCAATGACGCATC | 737 | CATAATATGGGTAAGACCTATCACCACAT |
| | ACGTGGAGTGTGTTGCTCTGCTCGTAAAA | | GTGGAGACGGTAGCACTTTTGTCCAAACT |
| | GCCTAGAAACC | | TGATGTCGA |
| 407 | GCTGGTGGTGGATATCGGCGGTGGTACGA | 738 | TCCATTAACTGTGGTGCACATCATAACAT |
| | CTGACTGTTCGTAGTCATGCAAGAATGTA | | AACTGTTCATTGCTGCTGATGGGGCCGCA |
| | ACCCGCAGTAA | | GTGGCGTTC |
| 408 | GGAGGCTAAAACCTTTTTTGCCTGATAAT | 739 | GGTGAAAATGTTGTAATAAGCGTCACACA |
| | CATACAAATGTGTTATGCTTATACAAACA | | CTCAAATAAGTGCCATTACAACAAATTGC |
| | AAAATTAGAAG | | AGGTGTATC |
| 409 | AGCTAAGTGTCCAAGCTGGCCCCCGATCC | 740 | TACATAATTTCGTATATTAGATATTACCA |
| | CAGTTTCAATTGGAAATACCTAATATACG | | GTTTCAATAGTTTGGGGAATCTTTGTAAG |
| | AAAAAAGGCG | | TGGGAGAC |
| 410 | ACAACAAAGACGCTAAGGTTTACGTGGTT | 741 | AATTAAACTAAGATATTTAGATACGCTAC |
| | AATGGAGACAAGAGTATCTAAATATCCTG | | TCGAGACAGTCGTCAAGATATTACAGGTT |
| | TTTTTTTCGC | | CATTTACA |
| 411 | CCCCAAAGTCGGCTTCGTCAGCCTTGGCT | 742 | GAAGTATAGGGTTTATTTCATTGGGGTGC |
| | GCCCGAAGGCCCTCTGAAGTAAACTCTTA | | CCGAAGGCCCTTGTTGATTCCGAGCGCAT |
| | TGACGCCCCG | | CCTCACCC |
| 412 | ATATCCCAAATGGAAAAGTTGTTAAACCG | 743 | AAAAATTTAGTTGGTTATTGGTTACTGTA |
| | TGTATAATCTTACGGTAACCAATAACCAA | | ACAAACGATACCAATCCCCCAACCTCCAA |
| | CTTTAAAACT | | GTGGATAT |
| 413 | AACGTTTGTAAAGGAGACTGATAATGGCA | 744 | ATGGATAAAAAAATACAGCGTTTTTCATG |
| | TGTACAACTATACTAGTTGTAGTGCCTAA | | TACAACTATACTCGTCGGTAAAAAGGCAT |
| | ATAATGCTTT | | CTTATGAT |
| 414 | GCCCAGGTGTGTCTGAGGTCATGGAAACG | 745 | CGCAGGTTCGAATCCTGCAGGGCGCGCCA |
| | GAAATCTTCAATTCCTGCACGACGACAAG | | TTTCTTCCTCATTTATGCCCGTCTTATCC |
| | CTGATAGCCAT | | GTTTCCGCT |
| 415 | TAACACCAATTAAGTGTTTAGTTCCCTCT | 746 | ATTTATAATTTTAGTTTCTCGTTTCTTCT |
| | TTGCGTCCAACGAGAGAAAACGAGGAACT | | TCTTCCCTCATAGCTTGATCCGAAAAAGT |
| | AAACAATCTAA | | TACAGCTGG |
| 416 | CTGAGTGGGCGAACTATTTATCTTTTACA | 747 | AATAATATTTTTATCCTTATTGACATATG |
| | ATGCCAATGCCATGTATAATTAGGGGATA | | AGGAAGCGGGTATAGCGGGAAGAAAGGAC |
| | AAAATAAAAA | | AAAATTTA |
| 417 | GAAACTATGGGGATTATAGCGTTTGAGGG | 748 | GAATAACTTTTTGCCGTATTGACATACCG |
| | AGCAAGTGCGGTGTATAATTAAGGCATAA | | CAAGTGCGGTTGGTAAGAGTAGCACGTGT |
| | AATAAAAAACG | | CGTGAATTA |
| 418 | CCGTCCCGCGACGGACCGAACCCAGTCGT | 749 | TATTGGTTAGGTGTCCTAGATCAACCTAC |
| | TGAGCCCGCTGTAAATCGGTCTATGACAT | | AGTCCCTTGTTCTCGTGAATCACCAATAC |
| | CTAACTAATA | | CGTGCCCC |
| 419 | AGACTCAAAAACTGCAACCTTAAAGCTTT | 750 | CTTCTTATTTAAACTAAGATATTTAGATA |
| | CACATTGCTTGAGATAAGAGTATCTAAAA | | CATTGCTTGAAAGCTTATTAACGCTATCA |
| | TTCACACTTTT | | GTAACAAGT |
| 420 | GACGACGTCAAATGAGAAATCTGTTACAC | 751 | TTTTTACAAAGAGGTATTTAGATACATGA |
| | GTGTAACAATGCCTGTATCTAAATACCTC | | GCTACATTAGCAGTTAACCGCCGTTTTAA |
| | TAAAGAAAGAC | | ATCGCAAAA |
| 421 | GTTAACAAGCACTTTAGACGGAATACAGC | 752 | ACATAAATATATGGAAGTATACACACTAT |
| | CATGGTTGGTTAATTGTGCATACTTCCAT | | ACATTTATGCATGTACCGCCATAGCTTTC |
| | AAAATATTAA | | TGTAAACT |
| 422 | AGAACTGCGCTTTTTACAACAAGAGCATT | 753 | TTTAGATTTTTCGTATTTACGATAACTTT |
| | TTGTTTGTGTAAACATAACATAAATACTA | | ACATGTTTATATTTAAATACAAAAAATCA |
| | ATAAAATGTTA | | AGTTATATA |
| 423 | TATAGGCTGACATAAGTGTACTGTGGCGA | 754 | TTTTCACTTCGTGTACATGGTGGAGTATT |
| | TTGTACTGGTTTAACTCTCTACCATGTAC | | AAACTGATTCACTTCCCCATACCCAAACA |
| | ACTTTTTTTC | | TATTACAC |
| 424 | TAAGGATAAGAAGGTTAAAGCATTTACAC | 755 | TCTGAATATCAATAATTTTAGTAACCTTG |
| | TTTTAGAAATCAAGGATAGTAAATTTCTT | | ATTGAGAGCCTTATTGTATTATCAGTAGT |
| | TATATTTTCC | | GGCATTTA |
| 425 | ATTCCAACCATCACCAAGAACATCTTTAC | 756 | AGATGCTCTCCCAGCTGAGCTAAACTCCC |
| | TTCCAAGTTCGATACCATTTGAAAACACA | | TAGAGCTAAGCGACTTCCCTATCTCACAG |
| | GGAGAACGAG | | GGGGCAAC |
| 426 | TCTGGCGGCAGTGCATTTCAAACACCATG | 757 | TGTGCTCTTTTATTGTAGTTATATAGTGT |
| | GTTTGGTCAATTAAACACAACCTAACTAC | | TTGGTCAATTGATGACTGGGCCACAGCTT |
| | ATTAAATAAA | | TTAGCTCA |
| 427 | TCCTAAGGGCTAATTGCAGGTTCGATTCC | 758 | AATCCCCTGCCGCTTCAAGTAGATGTCTG |
| | TGCAGGGGACACCATTTATCAGTTCGCTC | | CAGGGGACACCAGATACCCTTCAAACGAA |
| | CCATCCGTACC | | ATCTACCTT |
| 428 | AAATAGAAAAATGAATCCGTTGAAGCCTG | 759 | TAATGATTTTTAATGTTTCACGTTCAGCT |
| | CTTTTTTATACTAAGTTGGCATTATAAAA | | TTTTTATACTAACTTGAGCGAAACGGGAA |
| | AAGCATTGCTT | | GGTAAAAAG |
| 429 | GACGAAATAGATATTTTTTGTGGCCATTA | 760 | GATTTATGCTTTGTCGTCACCTTGTTGGT |
| | AGCGCATGAGGTTGTTACCAACAGGGTGA | | GTAATTAGATTTACCCCATTTAATCCTAA |
| | TAACAAAGCT | | AGCATCAT |
| 430 | AACGAAGTAGATGTTTTTTGTTGCCATTA | 761 | CGTTTATGCATTGTTGTCACCTTGTTGGT |
| | GGCGCATGAGGTTGACGACAACATGGTAG | | GTAATTAGATTTACCCCATTTAATCCTAA |
| | CGACAATATA | | TGCATCAT |
| 431 | AATATTAATAAGTTATATTGGGGGAACGT | 762 | TTTTTTTACGTGAATGTTTTGTAACAACT |
| | GTGCGGTCTACCGCGTAACACACCATTCA | | ACAGTAGAAGTGGTACCATTCATGTCCTT |
| | TCAAAATTTA | | ACGAGATA |
| 432 | ATCGCTGTAGCGCATAAATACGTTATGAG | 763 | GGTTTATAATTTTTGTCCCTATAAGCATA |
| | ACACGCAGATGCCGACAGACTATATAGAC | | CCGCAGATGCTGAAATTCGAGAAAAGAGC |
| | AAAAATAAAAC | | AAAGTAAAG |
| 433 | CATCTTTACTTTGCTCTTTTCTCGAATTT | 764 | AGTTTTATTTTTGTCTATATTGGCTGTCG |
| | CAGCATCTGCGGTATGCTTATAGGGACAA | | GCATCTGCGTGTCTCATAACGTATTTATG |
| | AAATTATAAAC | | CGCTACAGC |
| 434 | ATCCCATGATGAGCCGAGATGACATAACC | 765 | GTGGAAAATATAAAGAATTTTACTATCCT |
| | CACCATTTCAATTAAAGATACTAAATCTC | | ACATTTCATTGAATGTCATTCTCTCACCT |
| | TTGATTTTTGA | | TTATCAACC |
| 435 | TCAAAAGTTAAGGGTTAAAGCATTTACGC | 766 | CCTATTGAATGAGAGTTTTAGATACGCTT |
| | TTTTAGAATGTTTGGTATCTAAAACTCAC | | TTAGAATGTTTGGTAGCATTGGTTACAAT |
| | GCTTTTTTGA | | CACAGGAG |
| 436 | GTTACTATAGCTCAGATGATTAAGGGACA | 767 | AAACCATCAACAATTTTCCTCTGAGTGTC |
| | CAGCCTACTTCCCGTTTTTCCCGATTTGG | | ATTTAGGCTGTGTCCCTTAATTACGTAAG |
| | CTACATGACA | | CGTTGATA |
| 437 | GAATGATGCGTTGGGGCTTAATGGAGTAA | 768 | TCTTTTGTCATCACCCTGTTGGCGTCAAC |
| | ATCTAATTACACCAACAAGGTGACGACAA | | CTAATGCGCCTAATGGCTACAAAAGACAT |
| | AGCATAAACG | | CTACTTCG |
| 438 | GGATCAAAAAGAACGACGATTCTTTAGTG | 769 | TTTTCTTTTGTATCAAAATCAGTAGGAAC |
| | TTTTTGAAATAATCTTACTGAGTTTAATA | | ATAGATCCAACCATGGGTTCAGGTTCATT |
| | CAATGCCGTG | | GATGTTAA |
| 439 | GGAAATTAATGAGCCGTTTGACCACTGAT | 770 | CAGGGTTACTTTATACAACATTAATCTGT |
| | CTTTTTGAAAATAAAGAGCAATGTTGTAC | | ATTTGAAATTTCAGAAGTGGCGCATCATG |
| | ATCAAGATGCA | | GTCCAGAAG |
| 440 | GTCTTCTGGACCATGATGCGCCACTTCCG | 771 | TGTATCTTGATGTACAACATTGCTCTTTA |
| | AAATTTCAAATACAGAATAATGTTGCATA | | TTTTCAAAAAGATCAGTGGTCAAACGGCT |
| | TAATATTACTA | | CATTAATTT |
| 441 | GTCTTCTGGACCATGATGCGCCACTTCCG | 772 | TGTATCTTGATGTACAACATTGCTCTTTA |
| | AAATTTCAAATACAGAATAATGTTGCATA | | TTTTCAAAAAGATCAGTGGTCAAACGGCT |
| | TAATATCACTA | | CATTAATTT |
| 442 | GTCTTCTGGACCATGATGCGCCACTTCCG | 773 | TGTATCTTGATGTACAACATTGCTCTTTA |
| | AAATTTCAAATACAGATTAATGTTGTATA | | TTTTCAAAAAGATCAGTGGTCAAACGGCT |
| | AAGTAACCCTG | | CATTAATTT |
| 443 | GTCTTCTGGACCATGATGCGCCACTTCCG | 774 | TGTATCTTGATGTACAACATTACTCTTTA |
| | AAATTTCAAATACAGAATAATGTTGCATA | | TTTTCAAAAAGATCAGTGGTCAAACGGCT |
| | TAATATTACTA | | CATTAATTT |
| 444 | ACAATCAACAAAGATGTATGGCGGTACAT | 775 | TGATATAAGTACGGAAGTATAGACACTCG |
| | GCATTAATATTTAATGTGTATACTTCCGT | | ATTAATATCGGATGTATACCGACTAAAAC |
| | ATTATTGTTT | | ATTAATTC |
| 445 | ATGAATTAATGTTTTAGTCGGTATACATC | 776 | CTATAAAAATACGGAAGTATACACATTAA |
| | CGATATTAATCAAGTGTCTATACTTCCGT | | ATATTAATGCATGTACCGCCATACATCTT |
| | ACATAAGTTA | | TGTTGATT |
| 446 | ACAATCAACAAAGATGTATGGTGGTACAT | 777 | TAACATATGTACGGAAGTATAGACACTTG |
| | GCATTAATATTTAATGTGTATACTTCCGT | | ATTAATATCGGATGTATACCTACTAAAAC |
| | ATTTTTGTTT | | ATTAATTC |
| 447 | CTGTTTCAACAAATGATGCTCTTGGCCTT | 778 | AAATACATATTCTCTTGTTGTCATCATGT |
| | AATGGTGTAAACCTAATTACACCAAGAGG | | TGGTGTAAACCTTATGCGTTTAATGGCGA |
| | ATGACGACAAA | | CAAAACATA |
| 448 | AGAAAAAGTGAATGTATTCACTGTTGGCT | 779 | ATAATATAAAATACTGTTGTTCTATATGG |
| | GGATTGGAGTTGCAACACAACTACAAATG | | ATTGGAGTTGCATGCACTCACCCTCCTAT |
| | CAGTATAAAGG | | GCTAAGTGT |
| 449 | ATACGATTTCGGACAGGGGTTCGACTCCC | 780 | AGCAGGGCGATCCTGAGTTTAATCTGGCT |
| | CTCGCCTCCACCAGCAAAGGTCACAATCG | | CGCCTCCACCATTCAAATGAGCAAGTCGT |
| | TGTCGATGTCA | | AAAAACATA |
| 450 | AACCAGCTGTAACTTTTTCGGATCAAGCT | 781 | TTAGATTGTTTAGTTCCTCGTTTCCTCTC |
| | ATGAGGGAAGAAGAATAAACGAGATACCA | | GTTGGACGCAAAGAGGGAACTAAACACTT |
| | AAAAAGAACAT | | AATTGGTGT |
| 451 | TATGCAACCCGTCGATATGTTCCCGCAAA | 782 | ATAGTAGGAAGATACAGAGTGTACTCTCA |
| | CAGCTCACATCGAGTGTGTAGGACTGCTT | | ACGCACGTGGAAACCGTAGTACTCTTGCA |
| | ACACGTGTGGA | | GTTAAAAGA |
| 452 | TATCTTTTAACTGCAAGAGTACTACGGTT | 783 | TCCACACGTGTAAGCAGTCCTACACACTC |
| | TCCACGTGCGTTGAGAGTACACTCTGTAT | | GATGTGAGCTGTTTGCGGGAACATATCGA |
| | CTTCCTACTAT | | CGGGTTGCA |
| 453 | AACCAGCTGTAACTTTTTCGGATCGAGTT | 784 | TTAGATTATTTAGTACCTCGTTATCTCTC |
| | ATGATGGAAGAAGAAGAAACGAGAAACTA | | GCTGGACGTAAAGAGGGAACAAAGCATCT |
| | AAATTATAAAT | | AATAGGTGT |
| 454 | TTTTCCCCGAAAATCTTTAACACCGCTAT | 785 | TATTTTGGTAGTTTATAGAAGTAATTTCA |
| | CCGTTGATGTTCACTCCATTAATTACCAA | | GTTGATGTCCCAGCTCCTCCAAAGAAAAC |
| | AATTTAAAAA | | TAAATATT |
| 455 | GGATCAGAAGGTTAGGGGTTCGACTCCTC | 786 | AAATTTGTTAGGGTAAAAAAGTCATAGTT |
| | TTGGGTGCGCCATCGATTAACCCTAACTG | | GGGTGCGCCATTTAAAAATAATAATAAGA |
| | ATAAATAAAAA | | CTGTAGCCT |
| 456 | TTTTCCCCCGAAAATCTTTAACACCACTA | 787 | TTATTTTGGTAGTTTATAGAAGTAATTTC |
| | TCTGTTGATATTCACTCCATTAATTACCA | | AGTTGATGTCCCAGCTCCTCCAAAGAAAA |
| | AAAAAACAGG | | CTAAATAT |
| 457 | GTAAACTAAAATATGCCCAGACCCCATTG | 788 | TATGGAATTGTATCAATCTCGGCGTGGTT |
| | CGTTATCCGTTGCCACTCTGAAATTGATA | | TTGTCGATAATTTTTAGTTCTTCTGGTTT |
| | CAATGTAACA | | TAAATTAC |
| 458 | GTAAACTAAAATATGCCCAGACCCCATTG | 789 | TATGGAATTGTATCAATCTCGGCGTGGTT |
| | CGTTATCCGTTGCCACTCTGAAATTGATA | | TTGTCGATAATTTTTAGTTCTTCTGGTTT |
| | CAATGTAACA | | TAAATTAC |
| 459 | CTTGTGGATCACCTGGTTTTTCGTGTTCA | 790 | TGTCTCTTTTTATTAGGGTTTATATCAAC |
| | GATACACACATGTAAAGTAGACATAAACA | | TACACACATACGAAGTGCTCCTGAGAGAG |
| | GCAAAAATTTG | | AAAGCGCAT |
| 460 | GAAGGCAGACCATTAACAGGAAGGGATGG | 791 | TAAAGATCGTAAAAAAGAAATAGAGTTCC |
| | AGCATTTACACCATTTATAAAAAAGCTGC | | GAATTGACCTTACCCAGAAAAAGTGGAGA |
| | TGGAGGCAAG | | GAAAGAAA |
| 461 | GGAAATTAATGAGCCGTTTGACCACTGAT | 792 | TAGTAATATTATATGCAACATTATTCTGT |
| | CTTTTTGAAAATAAAGAGCAATGTTGTAC | | ATTTGAAATTTCGGAAGTGGCGCATCATG |
| | ATCAAGATACA | | GTCCAGAAG |
| 462 | GTCTTCTGGACCATGATGCGCCACTTCCG | 793 | TGTGTCTTGATGTACAACATTACTCTTTA |
| | AAATTTCAAATACAGAATAATGTTGCATA | | TTTTCAAAAAGATCAGTGGTCAAACGGCT |
| | TAATATTACTA | | CATTAATTT |
| 463 | GCTTCTGCTTGGATTTTACGCCATCCAGC | 794 | TTCATTATTTTAATAGAGATAGAAATCAA |
| | CAATATGCACATGGTAGCATGAGTGTTCT | | CCATGCAAGTGATCGCCGGTACGATGAAC |
| | ATGAAAAAAGA | | GTAGGGCGA |
| 464 | GTCTTCTGGACCATGATGCGCCACTTCCG | 795 | TGTATCTTGATGTACAACATTACTCTTTA |
| | AAATTTCAAATACAGAATAATGTTGCATA | | TTTTCAAAAAGATCAGTGGTCAAACGGCT |
| | TAATATTACTA | | CATTAATTT |
| 465 | AGCTTTTATTGCAAGAAAAATGGGTTATA | 796 | TATTTATATAAAATAGTGTTTTTGTAAAG |
| | AGTACACATCACCATATTTGACAAAAAAC | | TACACATCAGGTTATAGTAATATCGAAAA |
| | CTATAAATAA | | AGGAAGCG |
| 466 | AACCAGCTGTAACTTTTTCGGATCGAGTT | 797 | TTAGATTGTTTAGTATCTCGTTATCTCTC |
| | ATGATGGAGGGAGAAGAAACGGGATACCA | | GTTGGACGTAAAGAGGGAACAAAGCATCT |
| | AAAATAAAGAC | | AATAGGTGT |
| 467 | ACGTTTGTAAAGGAGACTGATAATGGCAT | 798 | TGGATAAAAAAATACAGCGTTTTTCATGT |
| | GTACAACTATACTCGTTGTAGTGCCTAAA | | ACAACTATACTCGTCGGTAAAAAGGCATC |
| | TAATGCTTTTA | | TTATGATGG |
| 468 | ACAATCATCAGATAACTATGGCGGCACGT | 799 | TTAATAAACTATGGAAGTATGTACAGTCT |
| | GCATTAATGTTGAGTGAACAAACTTCCAT | | TGCAACCACGGTTGTATCCCGTCTAAAGT |
| | AATAAAATAA | | ACTCGTAC |
| 469 | AACAATCTGCAAACATGTATGGCGGTACA | 800 | TTAATTTTTGTACGGAAGTAGATACTATC |
| | TGTATCAATATCCATGTTACTTAGTGCCA | | TTTCAACATTGGTTGTATTCCTACAAAGA |
| | TACAAAAACC | | CACTCATT |
| 470 | ACAGCCTGTGGATATGTTTGCACAGACTG | 801 | GTCTTTTTACCTTATATAACAGTTTCATG |
| | CTCACGTGGAGACGGTAGTATTGATGTCA | | CACGTGGAGTGTGTAGTTAAGCTAATCAA |
| | CGAAAAGAAAA | | GGTAAATCA |
| 471 | CGAGACGAGAAACGTTCCGTCCGTCTGGG | 802 | TGTTATAAACCTGTGTGAGAGTTAAGTTT |
| | TCAGTTGCCTAACCTTAACTTTTACGCAG | | ACATGGGCAAAGTTGATGACCGGGTCGTC |
| | GTTCAGCTTA | | CGTTCCTT |
| 472 | ATTCTCCTTTAACGAATGAAGCGACTAAT | 803 | TTGACTTTTGACATCAATACTACGCACTC |
| | TCGATATGGCTTGAGAGGACAGAATGAAT | | CACATGATGGGTTTGCGGGAAAAGATCTA |
| | GTCATTTGAGT | | CAGGCTGAA |
| 473 | CAGCCGGCTGATTTATTTCCAAATACGCA | 804 | TCCATAATATGGGTAAGACCTATCACCAC |
| | TCACGTGGAGTGTGTTGCTCTGCTTGTAA | | ACGTGGAGTGCGTAGTGTTGCTACAACGA |
| | AAGCTTAGAAA | | AGCAACGGG |
| 474 | TATGCAACCCGTCGATATGTTCCCGCAAA | 805 | ATAGTAGGAAGATACAGAGTGTACTCTCA |
| | CAGCTCACATCGAGTGTGTAGGACTGCTT | | ACGCACGTGGAAACCGTAGTACTCTTGCA |
| | ACACGTGTGGA | | GTTAAAAGA |
| 475 | AACAGAAGAAGGGAAGTTCTACCTATTGA | 806 | CCGAAGCATCGTATCAATGCTTCGGTCAA |
| | TACCTTTGGCAAAGGGCACGAGTTTGATA | | TGTTTGGTGGAGCTGAGGAGACGATATCT |
| | CAAAATGCACC | | AGAACCGAT |
| 476 | AACAGAAGAAGGGAAGTTCTACCTATTGA | 807 | CCGAAGCATCGTATCAATGCTTCGGTCAA |
| | TACCTTTGGCAAAGGGCACGAGTTTGATA | | TGTTTGGTGGAGCTGAGGAGACGATATCT |
| | CAAAATGCACC | | AGAACCGAT |
| 477 | AACAGAAGAAGGGAAGTTCTACCTATTGA | 808 | CCGAAGCATCGTATCAATGCTTCGGTCAA |
| | TACCTTTGGCAAAGGGCACGAGTTTGATA | | TGTTTGGTGGAGCTGAGGAGACGATATCT |
| | CAAAATGCACC | | AGAACCGAT |
| 478 | GTCTCGCTCGCCCACCGCGGGGTGCTCTT | 809 | GTAGCCACTTGTTTTACACGTCTTGTCTC |
| | TCTGGACGAGGCATGTAAAACAGGTGGGC | | TGGACGAGGCCCCGGAGTTCTCGGGGAAG |
| | TTGATCAGCTA | | GCGCTGGAC |
| 479 | CACTACAGTATGCAGATTTTGCAGCTTGG | 810 | TATGATAATTTTAGTATTCATGATTGGTT |
| | CAGCGTGAATAGCCCGTTATGAATACTAA | | GTTTGAATGGCTACAAGGTGAGGCGTTAG |
| | AAATTCCACTC | | AGCAACAGC |
| 480 | TCATCACTACTTAATATATCCATAAGAGA | 811 | ACCCTTAAACATATAACATGTTTAAGGGT |
| | AATTTCATTACCCACTTCATGTTGTATGT | | ATTCATTTCCTTCTTTGTCTACTCCTATA |
| | TATGTAAAAA | | GGATCTTG |
| 481 | TCTGGTGGCAGTGCATTTCAAACACCGTG | 812 | TGTGCTCTTTTGTTGTATTTATATGGCGT |
| | GTTTGGTCAATTAAACACAACCTAACTAC | | TTGGTCAATTGATGACTGGGCCACAGCTT |
| | ATCAAATGAA | | TTAGCTCA |
| 482 | GTTTTTTGTAGCCATTAGGCGCATGAGGT | 813 | GTCGTCACCTTGTTGGTGTAATTAGATTA |
| | TTACGCCAACAGGGTGATAACAAAAGAAG | | ACCCCATTAAGCCCTAAAGCGTCATTCGT |
| | GATTTTTTAAT | | CGAAACAGC |
| 483 | GATCACCCAGGACGTCTGCGCCTTCTACG | 814 | CCTGTATTGTGCTACTTAGAGCATAAGGC |
| | AGGACCATGCCTTACAAGCTCAAAATAGC | | GACCATGCCCTCTACGACGCCTACACGGG |
| | ACACGTTTCCG | | CGTGGTGGT |
| 484 | GCAACCGGCATCAGTGTAATACCGATAAT | 815 | CAAATAATGTAGTACCCAAATTAAGTTTC |
| | CGTAACAAGCAACCTTAATCGGGTACTAC | | ACACAACAGAGCCTGTCACGACCGGCGGA |
| | TTAATATCTA | | AAAAACGA |
| 485 | GTGAGGATGCGCTCGGAGTCGACCAGCGC | 816 | TCTGAGAATTAGTATATTTTCCTATTCGC |
| | CTTGGGGCACCCTAACGAAACCCATCCTA | | AGGGGCATCCAAGACTGACGAAGCCGACT |
| | TACTAGGGGC | | TTGGGAGT |
| 486 | ACAAGACCCCATCGGAACAGATAAAGAAG | 817 | ATACCAATAACATATAAAGAGTAGTGTGT |
| | GTAATGAAATAAACACTACTATTTATATG | | AATGAAATAAGTCTTTTAGATATACTTGG |
| | TTATTTTCTA | | CACAGAGG |
| 487 | GCTGGTGGTGGATATCGGCGGTGGTACGA | 818 | TCCATTAACTGTGGTGTACATCATAACAT |
| | CTGACTGTTCGTAGTCATGCAAGAATGTA | | AACTGTTCATTGCTGCTGATGGGGCCGCA |
| | CACCGCAGTAA | | GTGGCGTTC |
| 488 | CCATCATAAGATGCCTTTTTACCGACGAG | 819 | AAAGCATTATTTAGGCACTACAACTAGTA |
| | TATAGTTGTACATGAAAAACGCTGTATTT | | TAGTTGTACATGCCATTATCAGTCTCCTT |
| | TTTTATCCAT | | TACAAACG |
| 489 | CCACTCCCAAAGTCGGCTTCGTCAGTCTT | 820 | GCCCCTAGTATAGGATGGGTTTCGTTAGG |
| | GGATGCCCCTACGAATAGAAAAATATACT | | GTGCCCCAAGGCGCTGGTCGACTCCGAGC |
| | AATTCTCAGG | | GCATCCTC |
| 490 | CACTCCCAAAGTCGGCTTCGTCAGTCTTG | 821 | CCCCCAGTGTAGGATTTATATCACTAGGT |
| | GATGCCCCAACGAATAGAAAAGTAAACTA | | TGCCCCAAGGCGCTGGTCGACTCCGAGCG |
| | GCTTTCAGCG | | CATCCTCA |
| 491 | ACCAGCTGTAACTTTTTCGGATCAAGCTA | 822 | TAGATTGTTTAGTATCTCATTATCTCTCG |
| | TGAGGGACGGAGACGAATCGAGAAACTAA | | TTGGACGCAAAGAGGGAACTAAACACTTA |
| | AATTATAAATA | | ATTGGTGTT |
| 492 | AGTTCAGCCCGTGGATTTGTTTCCAATGA | 823 | TCGTTCCATAATATGGGTAAGACCTATCA |
| | CGCATCACATCGAGTGTGTGGTTCTGCTC | | CCACATGTGGAGTGCATAGCGTTGATACA |
| | GTAAAAGCCT | | AAGAGTGA |
| 493 | AGAAATCACTCAGCAAGAGTTAGCCAGGC | 824 | CCCCCTCGTGTTATTGTGGGTACATGATA |
| | GAATTGGCAACCCGAATGTAGTCAACCCA | | TTTGGCAAACCTAAACAGGAGATTACTCG |
| | AAATAACTAAA | | CCTATTTAA |
| 494 | CAGCCGACTGATTTGTTTCCGAATACGCA | 825 | ATATGACATCAATGCCATCAACTCGAGCC |
| | TCACGTGGAGTGTGTGGTTCTGCTCGTAA | | ACGTGGAGTGCGTAGTGTTGCTACAACGA |
| | AAGCCTAGAAA | | AGCAACGGG |
| 495 | GTCTTCTGGACCATGATGCGCCACTTCTG | 826 | TGTATCTTGATGTACAACATTGCTCTTTA |
| | AAATTTCAAATACAGATTAATGTTGTATA | | TTTTCAAAAAGATCAGTGGTCAAACGGCT |
| | AAGTAGCCCTG | | CATTAATTT |
| 496 | TGATTTGATTGTATTGGATATTATGTTAC | 827 | AATATAGTTGTATAAAAAGTCCTTTGCCA |
| | CAGATGGCGAAGGACTTTTTGTACAACAA | | GATGGCGAAGGTTATGATATTTGTAAAGA |
| | AAAGTCACAA | | AATAAGAA |
| 497 | AAAATGTGTAGACATGTTTCCTTATACGA | 828 | CGAAAGACATCAATACTGTCCTCTCGAGC |
| | CACATGTTGAGTGCGTCACATTGATGTCA | | CATGTTGAGACGGTAGTGTTAATGGAGAG |
| | AGGGTTTAGAA | | AAAGTAAGA |
| 498 | AATAACAAACTATTTTTTATAGAAACATG | 829 | AAAGAAAAAATTCTTTATTTCTACATACG |
| | GGGATGTCCGTATGTAGAAAATAGTAGGA | | GTTGTCAGATGAATGAAGAGGATTCCGAA |
| | ATATATGAGA | | AAATTATC |
| 499 | TAACACCAATTAAGTGTTTAGTTCCCTCT | 830 | CTTTATTTTTTTTGTATCCCATTTCCTCT |
| | TTGCGTCCAACGAGAGGAAATGAGGCACT | | CCCTCCCTCATAGCTTGATCCGAAAAAGT |
| | AAACCAGTTGA | | TACAGCTGG |
| 500 | TAACACCAATTAAGTGTTTAGTTCCCTCT | 831 | TGTTCTTTTTTTGGTATCTCGTTTCTTCT |
| | TTGCGTCCAACGAGAGAAAACGAGGTACT | | TCTTCCCTCATAGCTTGATCCGAAAAAGT |
| | AAATAAGCTAA | | TACAGCTGG |
| 501 | TAACACCAATTAAATGTTTAGTTCCCTCT | 832 | TGTTCTTTTTTTGGTATCTCGTTTCTTCT |
| | TTGCGTCCAACGAGAGAAAACGAGGTACT | | TCTTCCCTCATAGCTTGATCCGAAAAAGT |
| | AAATAAGCTAA | | TACAGCTGG |
| 502 | GGTGAGGATGCGCTCGGAGTCGACCAGCG | 833 | CTTAAAGATTGAGTTTACTTTTGCAGTCA |
| | CCTTGGGGCACCCTAACGAAACCCATCCT | | TTGGGGCATCCAAGACTGACGAAGCCGAC |
| | ATACTAGGGG | | TTTGGGAG |
| 503 | TTTATCCCGTAAGGACATGAATGGTACCA | 834 | TAAATTTTGATGAATGGTGTGTTACGCGG |
| | CTTCTACTGTAGTTGTTACAAAACATTCA | | TAGACCGCACACGTTCCCCCAATATAACT |
| | CGTAAAAAAA | | TATTAATA |
| 504 | TATCCCGTAAGGACATGAATGGTACCACT | 835 | AATATTAATGAGTGTTATGTAACTAGAAA |
| | TCTACCGCAATAGTTACAAAACATTCATT | | GACCGCACACGTTCCCCCAATATAACTTA |
| | AAAAATAACC | | TTAATATT |
| 505 | GGATCAAAAAGAACGACGATTCTTTAGTG | 836 | TTTTCTTTTGTATCAAAATCAGTAGGAAC |
| | TTTTTGAAATAATCTTACTGAGTTTAATA | | ATAGATCCAACCATGGGTTCAGGTTCATT |
| | CAATGCCGTG | | GATGTTAA |
| 506 | CACTCCCAAAGTCGGCTTCGTCAGTCTTG | 837 | CCCCTAGTATAGGATGGGTTTCGTTAGGG |
| | GATGCCCCAATGATTGCAAAAGTAAACTC | | TGCCCCAAGGCGCTGGTCGACTCCGAGCG |
| | AATCTTTAAG | | CATCCTCA |
| 507 | GTGGATCACCTGGTTTTTCGTGTTCAGAT | 838 | CTCTTTTTATTAGGGTTTATATCAACTAT |
| | ACAGGCATGTAAAGTAGACATAAACAGCA | | ACACATACGAAGTGCTCCTGAGACAGAAA |
| | AAAATTTGATA | | GCGCATATC |
| 508 | TCTATTTAAATTGTCTATTTTATTGACAG | 839 | AAGATATTACCCTGAATGAAGTCTTACGT |
| | GGGACCAATCTCTGCTAAGATTACCAAAT | | CGTCAAATTGAAGTGGCCGCTAATCAGTT |
| | AACCCCGACAA | | CCTTCAAAA |
| 509 | TCTATTTAAATTGTCTATTTTATTGACAG | 840 | AAGATATTACCCTGAATGAAGTCTTACGT |
| | GGGACCAATCTCTGCTAAGATTACCAAAT | | CGTCAAATTGAAGTGGCCGCTAATCAGTT |
| | AACCCCGACAA | | CCTTCAAAA |
| 510 | CCGAGCTGCCGATCACCGAGATCGCGTTC | 841 | TGGCCTCTCCTGAAGTGTCAGTTGAGCGC |
| | GCGTCCGGCTTTCCGAGTGCGCGTGAACT | | CTTCGGTTTCGCCAGCGTGCGGCAGTTCA |
| | ACAGTTCTAGC | | ACGACACGA |
| 511 | GATCACCCAGGACGTCTGCGCCTTCTACG | 842 | CCTGTATTGTGCTACTTAGAGCATAAGGC |
| | AGGACCATGCCTTACAAGCTCAAAATAGC | | GACCATGCCCTCTACGACGCCTACACGGG |
| | ACACGTTTCCG | | CGTGGTGGT |
| 512 | ACCAGCTGTAACTTTTTCGGATCAAGCTA | 843 | TACGTTGTTTAGTACCTCAATTTCTCTCT |
| | TGAGGGACGGAGACGAATCGAGAAACTAA | | CTGGACGCAAAGAGGGAACTAAACACTTA |
| | AATTATAAATA | | ATTGGTGTT |
| 513 | ACTGGCGAAGCGATTCTTGGTGCGAACAT | 844 | AAACCCATTTTTACCTTATGTAAAAAAAT |
| | TTTCCGTGATATGTTTACCAAATGACAAA | | CACGTGATTTTTTTGCGGGCATCCGTGAT |
| | AATGATATAAT | | GTGGTCGGC |
| 514 | TTCTAACTCACGACACGTTGTGCTCTTAC | 845 | GGTTTTTTATTTGTATGCCATAATTATAC |
| | CAACCGCACTTGCGGTATGTCAATAAGAC | | ACCGCACTCGCTCCCTCAAACGCTATAAT |
| | ATACGAATTT | | CCCCATAG |
| 515 | GGTGAGGATGCGCTCGGAGTCGACCAGCG | 846 | CTTAAAGATTGAGTTTACTTTTGCAGTCA |
| | CCTTGGGGCACCCTAACGAAACCCATCCT | | TTGGGGCATCCAAGACTGACGAAGCCGAC |
| | ATACTAGGGA | | TTTGGGAG |
| 516 | GCTGTGGCGGTTCCAAATTGGTGAGGCGC | 847 | AACGTGCCTTTGTCGCAGCTGCCAAAGTT |
| | CAAATCCGCTCAACTTGGTGGCGACCGAT | | TAGCCGACGTCCCCCCATCCTGAGTAGCA |
| | GCCTGCGGTCA | | GTCGGGTTT |
| 517 | AAAATCTAAATTTTCTTTTGGCAGACCTT | 848 | CCTTTAATTTTTGGGTTAAAGGAACATTG |
| | CTTCGCTAGTGAGTGTTATATTAACCCAA | | ACTCTACTCGTAATATTACCTAACACGGA |
| | AAAGAGCCTAC | | ACGAAATAA |
| 518 | TACAGACTTACATGGGACCATTCTATAGC | 849 | TCAACTTTTAACCCTGTTTTAAGACCCAG |
| | AGCTTTAAAATACTTAGCAATAAAACAGG | | TATTAAGATGCGTGAGGGACAAGATTACC |
| | GGAATTGATA | | AGACTCAG |
| 519 | ATCACGATGGGGAGCAGTTCGATGTACCC | 850 | TCCGTGATAGGCCGCGTGGCGTCGCCTCA |
| | CATCTCCACCACTTACCCAAAACCCAACC | | GCACCAGGTCCTTCACCACATAGTCCGCC |
| | CTTATCGGTTG | | GCCCCCTGC |
| 520 | GGTTAAGTGTATGGATATGTTCCCAAATA | 851 | ACTCAAATGACATTCATTCTGTCCTCTCA |
| | CTCCACACGTTGAGTGCGTAGTATTGATG | | AGCCATTGTGAGACGTGCGTACTTTTGTC |
| | TCAAGGGTTG | | CCACAAAA |
| 521 | AACCAGCTGTAACTTTTTCGGATCAAGCT | 852 | TCAACTGGTTTAGTGCCTCATTTCCTCTC |
| | ATGAGGGAAGAAGAAGAAACGAGATACCA | | GTTGGACGCAAAGAGGGAACTAAACACTT |
| | AAAAAAGAACA | | AATTGGTGT |
| 522 | CGTTTATGAATGACTTGATTTTTGGTATG | 853 | AGACATTCATTTTTATTAGGGTTTATGTA |
| | TAAAGTATAAGCATGTAAACTTAACATAA | | AAGTATAAGCAGACAAAATGCTCCTGGGA |
| | ATACAAATAA | | TAAAAAGC |
| 523 | TCTTCAAGATCCAATAGGAATAGATAAAG | 854 | AACATTTTACAAGTATATAACATGTAATA |
| | AAGGCAATGAATTACCCTGGACAAGTTGT | | GGCAATGAAATCTCTTTAATGGATGTTTT |
| | CAGTCTAGGG | | AGGTACAG |
| 524 | AACAGTTCCTTTTTCAATGTTACTGTAAC | 855 | TTATTTATAGGTTTTTTGTCAAATACGGT |
| | CTGATGTGTACTTTACAAAAACACTATTT | | GATGTGTACCTATAGCCCATCCGTCGCGC |
| | TATATAAATA | | AATGAAAG |
| 525 | GGGGCAAATTGCTGCGATTTGGGTTGGAG | 856 | AGAATAATTATATGTCTTCTATTGGCGGT |
| | GGGGAACCCCAGCATAGACAATATACATA | | AATACGTTGATTCCATGGGCGCTCATTCC |
| | TAATCTTTCT | | AGCTGCTG |
| 526 | GTCTTCTGGACCATGATGCGCCACTTCCG | 857 | TGTATCTTGATGTACAACATTGCTCTTTA |
| | AAATTTCAAATACAGAATAATGTTGCATA | | TTTTCAAAAAGATCAGTGGTCAAACGGCT |
| | TAATATTACTA | | CATTAATTT |
| 527 | ATGAATTAATGTTTTAGTCGGTATACATC | 858 | GGTTATTTTTACGGAAGTATACACATTAA |
| | CGATATTAATCAGGTGTCTATACTTCCGT | | ATATTAATGCATGTACCGCCATACATCTT |
| | ACATATGTTA | | TGTTGATT |
| 528 | GATGTTCGTAGCAACTATGGGAGGAACCG | 859 | GGTTTTTATATGTGCGTTATGTAACAAGC |
| | GTGCAACGGCTATAGTTACATAACCCACA | | ACCACATTAGTTGTTCCATTTATGTTTAT |
| | TTAAAATATA | | GTGGTTAA |
| 529 | ATGAATTAATGTTTTAGTCGGTATACATC | 860 | TTATTTTTTTACGGAAGTATACACAATAA |
| | CGATATTAATAGAGTGTCTATACTTCCGT | | ATATTAATGCATGTACCGCCATACATCTT |
| | ACATATGTTA | | TGTTGATT |
| 530 | ACAGTTTACAGAAAGCTATGGCGGTACAT | 861 | TTGATATTTTATGGAAGTATGCACAATTA |
| | GCATAAATGTATAGTGTGTGTACTTCCAT | | ACCAACCATGGCTGTATTCCGTCTAAAGT |
| | ATATTTATGC | | GCTTGTTA |
| 531 | ATAGAAGCACACTGATGATGAGCAAGACC | 862 | AATTGGAAAATATAAATAATTTTAGTAAC |
| | ACCAACATCTCAATAAAGGATAGTAAAAT | | CTACATTTCCACAAGTGTGAAAGCTTTAA |
| | TATTGATTTT | | CCTTAGCT |
| 532 | ACCAGCTGTAACTTTTTCGGATCAAGCTA | 863 | TACGTTGTTTAGTACCTCAATTTCTCTCT |
| | TGAGGGACGGAGACGAATCGAGAAACTAA | | CTGGACGCAAAGAGGGAACTAAACACTTA |
| | AATTATAAATA | | ATTGGTGTT |
| 533 | GGATTTCGTTGCACTGATGGGCGGTACTG | 864 | CTCTTTTTTATGTATGGTTTGTAACAATA |
| | GCGCGACCTACAAAGTGCTAAACCATACA | | TCCACTTTACTCGTTCCTTATTTATTTAT |
| | TGTTAAAAAT | | ATTTCTTT |
| 534 | GGATTTCATTGCACTGATGGGCGGTACTG | 865 | TCTTTTTTTATGTATGGTTTGTAACAATA |
| | GCGCGACCTACAAAGTGCTAAACCATACA | | TCCACTTTACTCGTTCCTTATTTATTTAT |
| | TGTTAAAAAT | | ATTTCTTT |
| 535 | TATATGTCTTCATATAATCGAGCAATGTG | 866 | TTAGGGTTACCATTGATCATGAAGACCAT |
| | TTCAGATCATCCAGCTCATAGTATTTTGT | | TATATAGTTGAGTCCGTATAATTGTGTAA |
| | CTCTTTCTTT | | AAAGCTAG |
| 536 | GCGCGCCGACTTTATGCAGGATCACATTG | 867 | TTCAAGTCTAGGATACGAACAGTACGTTT |
| | CTGGGCACACGATAACGTGCCGTTCGTAA | | GCGCACTTCGAACAGAAAGTAGCCGAGGA |
| | ACCGACGAGC | | AGAAGATG |
| 537 | TTCGTTAATTGGAGCTACGGCCATTGGTG | 868 | AGATGTGATGTTAATTATTCTGGTCAGTA |
| | GACCTCCTGACCGGATTAATTAATATCAC | | CCTCCTGACCACCCCCACTCGTAAGTCAT |
| | TAGGAAATGGC | | AATAATTAC |
| 538 | TAATGCATACATTGTCGTTGTCTTCCCAG | 869 | TTAATATCAGTTGTATTTATACTACTAGC |
| | AACCAGTAGCTAACGTTATATAAATACAC | | TCTGTCGGTCCAGTAAACACGAGTAGCCC |
| | TTAAAATAAA | | CTGTGAAT |
| 539 | GCTCTGCAAAAGCTTGATCGTCGGTTCAA | 870 | AAACCCTTGATATACCAATAGTTTCAAAT |
| | ATCCGTCTACCGCCTTTATTATAGGATTT | | CCGTCTACCGCCTTTTAATATTCTAAAAA |
| | TGTCCGAATT | | ACCTAGGA |
| 540 | ACAATCATCAGATAACTATGGCGGCACGT | 871 | TTAATTTAGTATGGAAGTATGCACAATTG |
| | GCATTAATGTATAATGTGTGTACTTCCAT | | AGCAACCACGGTTGTATCCCGTCTAAAGT |
| | ATATTTATAC | | ACTCGTAC |
| 541 | ATGTACGAGTACTTTAGACGGGATACAAC | 872 | GTATAAATATATGGAAGTACACACATTAT |
| | CGTGGTTGCTCAATTGTGTATACTTCCAT | | ACATTAATGCACGTGCCGCCATAGTTATC |
| | ACTAAATTAA | | TGATGATT |
| 542 | ATGAAGATTATAATAATTGGAGGTGGCTG | 873 | TCACGTGTTTTAATGGAGTTTTAACTGGT |
| | GTCTGGATGTGCAGCACAGGTAAAACTAC | | CTGGATGTGCAGCAGCCATAACAGCTAAA |
| | ACTAATTATTA | | AAGGCAGGT |
| 543 | AACCCCAAAGTCGGCTTCGTCAGCCTTGG | 874 | TAGAAGTATAGGGTTTGTTTCATTGGGGT |
| | CTGCCCGAAGGATGGTTGAGATATACTTT | | GCCCGAAGGCCCTCGTCGATTCCGAGCGC |
| | TGGCGAGCAG | | ATCCTCAC |
| 544 | GAATCTAAATTTTCTTTCGGTAATCCTTC | 875 | CTTTAATTTTTGGGTTAAAGGAACATTGA |
| | TTCACTACTAAGTGTTATATTAACCCAAA | | CTCTACTCGTAATATTTCCTAATACAGAA |
| | AAAGAGCCTTC | | CGAAATAAA |
| 545 | CTGGCTTGATTAATAGTTTAAAAGTCTTG | 876 | TCCTGAATGGTTACTACGATTGGTTTGGT |
| | GCTGGTGTTATTGCTGTGAATAAAGTTGT | | TGGTGTCACGAACGGTGCAATAGTGATCC |
| | TGGTGTAACCA | | ACACCCAAC |
| 546 | CACTCCCAAAGTCGGCTTCGTCAGTCTTG | 877 | CCCCTAGTATAGGATGGGTTTCGTTAGGG |
| | GATGCCCCAACGAATAGAAAAGTAAACTA | | TGCCCCAAGGCGCTGGTCGACTCCGAGCG |
| | GCTTTCAGCG | | CATCCTCA |
| 547 | GGTGAGGATGCGCTCGGAGTCGACCAGCG | 878 | CTTAAAGATTGAGTTTACTTTTGCAGTCA |
| | CCTTGGGGCACCCTAACGAAACCCATCCT | | TTGGGGCATCCAAGACTGACGAAGCCGAC |
| | ATACTAGGGG | | TTTGGGAG |
| 548 | CACTCCCAAAGTCGGCTTCGTCAGTCTTG | 879 | CCCCTAGTATAGGATGGGTTTCGTTAGGG |
| | GATGCCCCAACGAATAGAAAAGTAAACCA | | TGCCCCAAGGCGCTGGTCGACTCCGAGCG |
| | GTTTTCAGCG | | CATCCTCA |
| 549 | GGTTAAGTGTATGGATATGTTCCCAAATA | 880 | ACTCAAATGACATTCATTCTGTCCTCTCA |
| | CTCCACACGTTGAGTGCGTAGTATTGATG | | AGCCATTGTGAGACGTGCGTACTTTTGTC |
| | TCAAGGGTTG | | CCACAAAA |
| 550 | AGCTTTCATTGCGCGACGGATGGGCTATA | 881 | TTTTTATATAATATAGTGTTTTTGTTAAG |
| | GGTACACATCACTATATTTGACAAAAAGT | | TACACATCAGGATACAGTAACATTGAAAA |
| | CTATAAATAA | | AGGAACTG |
| 551 | CGCATGTTCGCGGCCGGCACGCTGGTCAC | 882 | GCCCTGTTAATATGTATATTGGCTAACGC |
| | GCTCGGCAACCCGAACGTTAGCCAATATA | | TCGGCAACCCGAAGATCATGCTGTTCTAT |
| | CAAACCATGCT | | CTGGCATTG |
| 552 | CGCATGTTCGCGGCCGGCACGCTGGTCAC | 883 | GCCCTGTTAATATGTATATCGGCTAACGC |
| | GCTCGGCAACCCGAACGTTAGCCAATATA | | TCGGCAACCCGAAGATCATGCTGTTCTAT |
| | CAAACCATGCT | | CTGGCGTTG |
| 553 | GGGTGGAAATAATATAAAAGGTGGCCTTA | 884 | AAATTTATAGTGAGGGTTTGTCATAGACA |
| | TAGGTCCTCCAATAAGATACAAGAACACA | | AGACCTGGAGTTCACGCTTCACATGGTAT |
| | ACGGCTTAAAA | | GGAGAGAAC |
| 554 | TTTTCCCCCGAAAATCTTTAACACCACTA | 885 | TTATTTTGGTAGTTTATAGAAGTAATTTC |
| | TCTGTTGATATTCACTCCATTAACTACCA | | AGTTGATGTCCCAGCTCCTCCAAAAAAAA |
| | AAATAAAAAA | | CTAAATAT |
| 555 | TATCTTTTAACTGCAAGAGTACTACGGTT | 886 | TCCACACGTGTAAGCAGTCCTACACACTC |
| | TCCACGTGCGTTGAGAGTACACTCTGTAT | | GATGTGAGCTGTTTGCGGGAACATATCGA |
| | CTTCCTACTAT | | CGGGTTGCA |
| 556 | ATCTTTTAACTGCAAAAGTACTACGGTCT | 887 | TTACCCTAGACATCAATGCTACCAACTCA |
| | CTACATGGGACGAGTTGATAGAATTGATG | | ACATGAGCTGTTTGCGGGAACATATCGAC |
| | TATTTGCGAT | | TGGTTGCA |
| 557 | TAAGGGCATGGACATGTTTCCTCATACAC | 888 | GAAATGACGTACTTTTCATTTCCTCGTGC |
| | CTCATGTGGAGACGGTGGTATTGATGTCA | | CATGTGGAAACTGTAGTTAAGCTAAGCAA |
| | AGGGCGGAGA | | ATAATATC |
| 558 | GCTGGTGGTGGATATCGGCGGTGGTACGA | 889 | TCCATTAACTGTGGTGTACATCATAACAT |
| | CTGACTGTTCGTAGTCATGCAAGAATGTA | | AACTGTTCATTGCTGCTGATGGGACCGCA |
| | CACCGCAGTAA | | GTGGCGTTC |
| 559 | ATAATCATCAAAGAGTTTAGGATTATCAA | 890 | TACTTTAATTTTAGGTTAATGGTCCATTT |
| | ATTCACTAGTAAATGTTATATTAACCCAA | | CCTCTATGATACGCCCTTCCGAAAGCTGA |
| | AAAAAAGAGTC | | TACTAACGA |
| 560 | ACCAGCTGTAACTTTTTCGGATCAAGCTA | 891 | CACATTATTTAGTTCCTCGTTTTCTCTCG |
| | TGAGGGACGGAGAATAAATGAGAAACTAA | | CTGGACGCAAAGAGGGAACTAAACACTTA |
| | AATACAAATAA | | ATTGGTGTT |
| 561 | AACAATCTGCAAACATGTATGGCGGTACA | 892 | ATTAATTTTGTACGGAAGTAGATACTATC |
| | TGTATCAATATCCATGTTACTTAGTGCCA | | TTTCAACATTGGTTGTATTCCTACAAAGA |
| | TACAAAAACC | | CACTCATT |
| 562 | AGGGCCTGGCTGCTGAACTCGGGCGTCTC | 893 | TCGCGGCCCACTTGCTTTACACGTCTCGT |
| | GTCGAGGAACGAGACGTATAAAACAAGTG | | CCAGGAAGAGGACGCCCCGGTGGGACAGG |
| | GCTACGGCCAG | | GACACCGCG |
| 563 | ACAATCAACAAAGATGTATGGTGGTACAT | 894 | TAACGTATGTACGGAAGTATAGACACCTG |
| | GCATTAATATTTAATGTGTATACTTCCGT | | ATTAATATCGGATGTATACCTACTAAAAC |
| | ATTTTTTATA | | ATTAATTC |
| 564 | ATGGCTGTTGCGTTGATAGCGCCAAGCGT | 895 | GTTTTTTTGTTTGCGTTAAATGGAATTAT |
| | TACTAGTAGGACATTTCCTAAAAGTGGCT | | CCAGTACGGCATATGCAGTAGAAACAACG |
| | AATTTTTTGT | | AGTCAACA |
| 565 | TATCTTTTAACTGCAAGAGTACTACGGTT | 896 | TCTTGGCGAGTGAGCAGACCTATACACTC |
| | TCCACGTGCGTTGACTGTCTACTTAGTAT | | GATGTGAGCTGTTTGCGGGAACATATCGA |
| | CTTCCTACTAT | | CGGGTTGCA |
| 566 | ATTAACAAGCACTTTAGATGGAATACAGC | 897 | GCATAAATATATGGAAGTACACACACTAT |
| | CATGGTTGGTTAATTGTGCATACTTCCAT | | ACATTTATGCATGTACCGCCATAGCTTTC |
| | AAAATATTAA | | TGTAAATT |
| 567 | GACCACAATCCGCGTGTGGGCTTTGTATC | 898 | GAAGCCGTATAGTATAGGAATGGTGTCGC |
| | CCTTGGGTGCCCGAGTGATGCTTAAAATA | | TTGGGTGCCCCAAGGCACTCGTCGATTCG |
| | CACTCGGTGCT | | GAGCAGATC |
| 568 | TTCGACGAATGATGCTTTAGGGCTGAATG | 899 | TTCATTAGCTTTGTTATCACCCTGTTGGT |
| | GAGTAAATCTAATTACACCAACAAGGTGA | | AACAACCTCATGCGCCTAATGGCTACAAA |
| | CAACAAAGCA | | AAACATCT |
| 569 | CAAAAATTGCAGTGCGTTCAGCGATGACA | 900 | TTTCTGCATTGTCCTATTATAATTATGAG |
| | GGACATTTGGTCATTATAATAGACCTATA | | CCATTTGATCGCTTCGACGATGCATACGA |
| | CACATAAACA | | AAGACGCT |
| 570 | AATTTTCTTGTCGATTGGCTATTCGACTT | 901 | TATTCTTAGTGGGGCTTAAGTCAACTTGT |
| | GTCATTGGTGTCATGTTTTCTTAAGCCTC | | CATTGGTGTCATGTGATGGAGAGAGAATC |
| | AAAATAAAAA | | TTTTGAGG |
| 571 | TTTTAAAATGATTAAAGGCGGCGTTCCAA | 902 | CTATTAATTGGGGGTATGTCTTACTTATT |
| | TAAGCGTACCTATTTCGCACCCCCAATAA | | AGCGTACCCAAGCCCCCAATAGTGCCGGC |
| | ACACCCCACC | | ATAACCGA |
| 572 | GGGTGAGGATGCGCTCGGAATCGACAAGG | 903 | CATCTACCGCAAAGTATAGGTATTTAATC |
| | GCCTTCGGGCACCCCAATGAAACAAACCC | | CTTCGGGCAGCCAAGGCTGACGAAGCCGA |
| | TATACTTCTA | | CTTTGGGG |
| 573 | AGCAACCCCCCTGCTGTTGGGCTTAACGT | 904 | TCAAAAAAGCGTGAGTTTTAGATACCAAA |
| | GCTTCTCTAAAAGCGTATCTAAAACTCTC | | CATTCGATGAAAGTGATACTGAGCCTGAG |
| | ATTCAATAGG | | AAATTAGA |
| 574 | CCATCATAAGATGCCTTTTTACCGACGAG | 905 | AAAGCATTATTTAGGTACTACAACTAGTA |
| | TATAGTTGTACATGAAAAACGCTGTATTT | | TAGTTGTACATGCCATTATCAGTCTCCTT |
| | TTTTATCCAT | | TACAAACG |
| 575 | CCAGATCAGTGCGCCCCCGGCGGTCCAGA | 906 | AAATCCTCCCTTTTACATCTGTACGGGCT |
| | GCAGGAAGCAGGCACGTACGGTTGTAAAA | | TGGAAGCGGACATGGCCCATGCGGAAGAG |
| | GGAAATCCTA | | GCCCGCTG |
| 576 | TAACACCAATTAAGTGTTTAGTTCCCTCT | 907 | TCTTTATTTTTTTGTATCCCATTTCCTCT |
| | TTGCGTCCAACGAGAGAAAACGAGAAACT | | CCCTCCCTCATAGCTTGATCCGAAAAAGT |
| | AAACAATCTAA | | TACAGCTGG |
| 577 | AACAGTTCCTTTTTCAATGTTACTGTAAC | 908 | TTATTTATAGACTTTTTGTCAAATATAGT |
| | CTGATGTGTACTTTACAAAAACACTATTT | | GATGTGTACCTATAGCCCATCCGTCGCGC |
| | TATATAAATA | | AATGAAAG |
| 578 | GTGAATGATTTGGTTTTTAATATTTAAAA | 909 | TTTAATTTATTCGTATTTACGTTACCTTC |
| | AAAGAACTACTAACTTCACATAAACCCAA | | ACTACAACAAAATGTTCCTGATTAAGTGA |
| | ACTTTTTACA | | AGTCATGT |
| 579 | GTGGATCACCTGGTTTTTCGTGTTCAGAT | 910 | CTCCTTTTATTAGGGTTTGTGTCATCTAC |
| | ACAGGCATGTAAAGTTTACATAAACCCTA | | ACACATACGAAGTGCTCCTGAGACAGAAA |
| | AAAAGATCGAC | | GCGCATATC |
| 580 | ACTTTTTATATTGCAAAAAATAAATGGCG | 911 | AGTGTGGTTGTTTTTGTTGGAAGTGTGTA |
| | GACGAGGTAACAGCATAGTTATTCCGAAC | | TCAGGTATCAGGATACCTCATCTGCCAAT |
| | TTCCAATTAAT | | TAAAATTTG |
| 581 | TAACACCAATTAAGTGTTTAGTTCCCTCT | 912 | ATGTTCTTTTTTTGTATCTCGTTTCTTCT |
| | TTGCGTCCAACGAGAGAAAACGAGGAACT | | TCTTCCCTCATAGCTTGAACCGAAAAAGT |
| | AAACAATCTAA | | TACAGCTGG |
| 582 | AGATAAAACACTCTCCAGGAAACCCGGGG | 913 | TGAGACAAACAGCCATGGCTGGTTCCCGG |
| | CGGTTCATACAATTATTTGTTATTGTGCA | | ATACAGATGGCGCACTCATCACCGGACTG |
| | TCATTCTGGT | | ACCTTTCT |
| 583 | ATATGTTCCCGCAAACAGCTCACGTTGAG | 914 | TATCCCCTCCTCTCAAAACATGTAGAGAC |
| | ACGGTAGTATTGATGTCAAGGGTAGATAA | | CGTAGTACTTTTGCAGTTAAAAGATAAAT |
| | GTAAGAGTGT | | AAAGGACT |
| 584 | ATATGTTCCCGCAAACAGCTCACGTTGAG | 915 | TATCCCCTCCTCTCAAAACATGTAGAGAC |
| | ACGGTAGTATTGATGTCAAGGGTAGATAA | | CGTAGTACTTTTGCAGTTAAAAGATAAAT |
| | GTAAGAGTGT | | AAAGGACT |
| 585 | AACCAGCTGTAACTTTTTCGGATCAAGCT | 916 | TTAGCTTATTTAGTACCTCGTTTTCTCTC |
| | ATGAGGGAAGAAGAATAAACGAGATACCA | | GTTGGACGCAAAGAGGGAACTAAACACTT |
| | AAAAAGAACAT | | AATTGGTGT |
| 586 | TGTTAACCACATAAACATAAATGGTACAA | 917 | TAAATTTTAATAGCAGTTGTGTCACTATT |
| | CTAATGTCTATCGTGTGACAAAACTAACA | | TAGGTGGCACCTGTACCACCCATAGTTAC |
| | TACAAAAACC | | CACGAACA |
| 587 | AAATGTTCGTTGCAACTATGGGGGGTACC | 918 | AGTTTTATACATAAAAATAGTGTAACAAG |
| | GGTGCTACCTACCCTGTAACACTACTACC | | CACTACATTAGTCGTTCCATTTATGTTTA |
| | ATTAAAATTT | | TGTGGTTA |
| 588 | ATAATGCAACATAGTCTCCAGTACCACCT | 919 | AAAAAAAGGCGCTCTTTGATGTAGCGCCC |
| | TTATATGCTCACTACATGAAAAAGCGATA | | ATATGCACCAGCAGTTGCTGAAAAATCTA |
| | ATTTTAAGTA | | TATTTGTT |
| 589 | ACCAGCTGTAACTTTTTCGGATCAAGCTA | 920 | TAGATTGTTTAGTTCCTCGTTTCCTCTCG |
| | TGAGGGACGGAGAATAAATGAGATACTAA | | TTGGACGCAAAGAGGGAACTAAACACTTA |
| | TCCATAATAAT | | ATTGGTGTT |
| 590 | AACCAGCTGTAACTTTTTCGGATCAAGCT | 921 | TTAGATTGTTTAGTTCCTCGTTTTCTCTC |
| | ATGAGGGAAGAAGAAGAAACGAGATACCA | | GTTGGACGCAAAGAGGGAACTAAACACTT |
| | AAAAAGAACAT | | AATTGGTGT |
| 591 | ATGAATTAATGTTTTAGTAGGTATACATC | 922 | GGTTATTTTTACGGAAGTATACACATTAA |
| | CGATATTAATCAGGTGTCTATACTTCCGT | | ATATTAATGCATGTACCACCATACATCTT |
| | ACATATGTTA | | TGTTGATT |
| 592 | AGCTGCGCGCGCAGTATTTCTCGAAGGAG | 923 | ATGACTTCGATAGTTAATTATGAAACACT |
| | CCCATGGATATAGGTGCATCAAAATTAAC | | CTTGGATCCGGACGTATCCATCATGGCGA |
| | TAAAGGAAAA | | TAATGACC |
| 593 | TCATCACTACTTAATATATCCATAAGAGA | 924 | TGCGTTAGGTGTATATCATGCCTAGCGCA |
| | AATTTCATTACATCATACATGTTGTACAC | | ATTCATTTCCTTCTTTATCTACTCCTATA |
| | CTACTTTAAA | | GGATCTTG |
| 594 | AACCAGCTGTAACTTTTTCGGTTCAAGCT | 925 | TTAGCTTGTTTAGTACCTCGATTTCTCTC |
| | ATGAGGGAGGGAGAAGAAACGGGATACCA | | GTTGGACGCAAAGAGGGAACTAAACACTT |
| | AAAATAAAGAC | | AATTGGTGT |
| 595 | AACCAGCTGTAACTTTTTCGGATCAAGCT | 926 | TCAACTGGTTTAGTGCCTCATTTCCTCTC |
| | ATGAGGGAAGAAGAAGAAACGAGATACCA | | GTTGGACGCAAAGAGGGAACTAAACACTT |
| | AAAAAAGAACA | | AATTGGTGT |
| 596 | ATGAAGGACTTGATTTTTAGTATTGAGAT | 927 | AGAATTTTATTAGTATTTATGTCAGGTTT |
| | AAAGACATGTAAACATAACATAAACACAA | | AAGCAAACGAAATTTTCCTGTTGTAAAAA |
| | AAAATCTTAT | | CCTCATAT |
| 597 | TCCCCGTGTCGGCGGTTCGATTCCGTCCC | 928 | TATGTGGGTTTGGTTTTCTGTTAAACTAC |
| | TGGGCACCAAAATTCAGCGCCCAACTGTT | | ACCACCATGAATACGACGAAAAGGCTCAC |
| | CTCAGTTGGGC | | CTCCGGGTG |
| 598 | TCCCCGTGTCGGCGGTTCGATTCCGTCCC | 929 | TATGTGGGTTTGGTTTTCTGTTAAACTAC |
| | TGGGCACCAAAATTCAGCGCCCAACTGTT | | ACCACCATGAATACGACGAAAAGGCTCAC |
| | CTCAGTTGGGC | | CTCCGGGTG |
| 599 | AACCAGCTGTAACTTTTTCGGATCAAGCT | 930 | TTAGATTGTTTAGTATCTCGTTATCTCTC |
| | ATGAGGGAGGGAGAAGAAACGGGATACCA | | GTTGGACGCAAAGAGGGAACTAAACACTT |
| | AAAATAAAGAC | | AATTGGTGT |
| 600 | GGTGAGGATGCGCTCGGAGTCGACCAGCG | 931 | CGCTGAAAGCTAGTTTACTTTTCTATTCG |
| | CCTTGGGGCACCCTAACGAAACCCATCCT | | TTGGGGCATCCAAGACTGACGAAGCCGAC |
| | ATACTAGGGG | | TTTGGGAG |
| 601 | GAGTTCTCTCCATACCATGCGAAGCGTGA | 932 | ATTCTTTAAAAAGAGTTCTCGTATTTTAT |
| | ACTCCAGGTCTTGTCTATGACATACCCTC | | TGGAGGACCTATAAGGCCACCTTTTATAT |
| | ACTATAAATTT | | TATTTCCAC |
| 602 | GAAAGTTTTTCTGAATCCTCTTCATTCAT | 933 | TTCTCTAATCTTCTTTATTTCTACATACG |
| | TTGGCAACCGTATGTAGAAATAAAGAAGT | | GTCAACCCCAGGTTTCTATGAAAAATTCA |
| | ATTGAGTAGTA | | CCTATAACA |
| 603 | AGCCTCTGTGCCAAGTATATCTAAAAGAC | 934 | TAGAAAATAACATATAAAAAGTAGTGTTT |
| | TTATTTCATTACACACTACTCTTTATATG | | ATTTCATTACCTTCTTTATCTGTTCCGAT |
| | TTATTGGTAT | | AGGGTCTT |
| 604 | AGGCAGATCACCTGTAACCCTTCGATTAT | 935 | AGGCCAGAGCAGCGTCTGGCCTTTAAATA |
| | TCTTGGTGGTGGAATGGCGACGAAATAAA | | ATGGTGGAGCGGAGGAGGATCGAACTCCC |
| | AACCCAAAAT | | GACCTTCG |
| 605 | GTCTTCTGGACCATGATGCGCCACTTCCG | 936 | TGTATCTTGATGTACAACATTGCTCTTTA |
| | AAATTTCAAATACAGATTAATGTTGTATA | | TTTTCAAAAAGATCAGTGGTCAAACGGCT |
| | AAGTAACCCTG | | CATTAATTT |
| 606 | TATGCAACCCGTCGATATGTTCCCGCAAA | 937 | ATAGTAGGAAGATACTAAGTAGACAGTCA |
| | CAGCTCACATCGAGTGTGTAGGACTGCTT | | ACGCACGTGGAAACCGTAGTACTCTTGCA |
| | ACACGTGTGGA | | GTTAAAAGA |
| 607 | GTTAACAAGCACTTTAGACGGAATACAGC | 938 | ACATAAATATATGGAAGTACACACACTAT |
| | CATGGTTGGTTGATTGTGCATACTTCCAT | | ACATTTATGCATGTACCGCCATAGCTTTC |
| | AAAATATTAA | | TGTAAACT |
| 608 | GAATGATGCGTTGGGGCTTAATGGAGTAA | 939 | TATATTGTCATCACCCTGTTGGCGTCAAC |
| | ATCTAATTACACCAACAAGGTGACGACAA | | CTAATGCGCCTAATGGCTACAAAAGACAT |
| | AGCATAAACG | | CTACTTCG |
| 609 | GTATTATTAGGGGTGTTTGCAATCGGGGC | 940 | TACATATTTTCATTATAATTTAAAGACGG |
| | ACCAGGAGTACGAGGTGTCTTTAAATAGT | | TAGGAGTCCCTGGGGGGACAGTAATGGCA |
| | TATGAAATTA | | TCATTAGG |
| 610 | GAAGAGCACCGAGCGCAGGAAGAGCGTGT | 941 | GGTCAGGCGGCACCTAGGGGGGTGGTTAA |
| | ACTGCTCCCATGAGCGTTGCGCACACCCT | | CGCTCCCACGCCGTCCACTCCGTGATGCG |
| | AATGTTGCCTC | | CCGGTCCGA |
| 611 | CAGCCGGCTGATTTATTTCCAAATACGCA | 942 | TCCATAATATGGGTAAGACCTATCACCAC |
| | TCACGTGGAGTGTGTTGCTCTGCTTGTAA | | ACGTGGAGTGCGTAGTGTTGCTACAACGA |
| | AAGCTTAGAAA | | AGCAACGGG |
| 612 | CAGCCGACTGATTTGTTTCCGAATACGCA | 943 | ATATGACATCAATGCCATCAACTCGAGCC |
| | TCACGTGGAGTGTGTGGTTCTGCTCGTAA | | ACGTGGAGTGCGTAGTGTTGCTACAACGA |
| | AAGCCTAGAAA | | AGCAACGGG |
| 613 | AACCAGCTGTAACTTTTTCGGATCAAGCT | 944 | TTAGATTGTTTAGTTCCTCGTTTTCTCTC |
| | ATGAGGGAGGGAGAAGAAACGGGATACCA | | GTTGGACGCAAAGAGGGAACTAAACACTT |
| | AAAATAAAGAC | | AATTGGTGT |
| 614 | AGTTCAGCCCGTGGATTTGTTTCCAATGA | 945 | TCGTTCCATAATATGGGTAAGACCTATCA |
| | CGCATCACATCGAGTGTGTGGTTCTGCTC | | CCACATGTGGAGTGCATAGCGTTGATACA |
| | GTAAAAGCCT | | AAGAGTGA |
| 615 | CGGGCAAATTGCTGCCATATGGACCGGAG | 946 | CTATTTATTAGATGTCTAAACAGTGCATT |
| | GCGGGACTCTACAACCTATATTAGACATC | | ACTACTTTAATTCCTTGGGCGCTTATTCC |
| | TTATAAAAAGT | | TGCCGCTGC |
| 616 | GTAACACCAATTAAGTGTTTAGTTCCCTC | 947 | TATTTATAATTTTAGTTTCTCGATTCGTC |
| | TTTGCGTCCAGCGAGAGATAACGAGGTAC | | TCCGTCCCTCATAGCTTGATCCGAAAAAG |
| | TAAATAATCTA | | TTACAGCTG |
| 617 | TCTAACTCACGACACGTTGTACTCTTACC | 948 | CAGTTTTTATTTTATGCCTTAATTATACA |
| | AACCGCACTTGCGGTATGTCAATATGGCA | | CCGCACTTGCTCCCTCAAACGCTATAATC |
| | AAAAGCTATTC | | CCCATAGTT |
| 618 | AGGCAGATCACCTGTAACCCTTCGATTAT | 949 | AGGCCAGAGCAGCGTCTGGCCTTTAAATA |
| | TCTTGGTGGTGGAATGGCGACGAAATAAA | | ATGGTGGAGCGGAGGAGGATCGAACTCCC |
| | AACCCAAAAT | | GACCTTCG |
| 619 | AGCAGGATGGAGATAACGAGCATGACGAC | 950 | AAACAAAAATAAGGGGTTATTACCCCTAT |
| | TAACATTTCAATAAATATGGGTAATAACC | | TTATTTCTATCAGTGTAAATCCCTTTTCA |
| | CTTAAATGATT | | TTCACAGTT |
| 620 | CTTGTGGATCACCTGGTTTTTCGTGTTCA | 951 | TGTCTCTTTTTATTAGGGTTTATATCAAC |
| | GATACACACATGTAAAGTAGACATAAACA | | TACACACATACGAAGTGCTCCTGAGAGAG |
| | GCAAAAATTTG | | AAAGCGCAT |
| 621 | ATATCCCAAATGGAAAAGTTGTTAAACCG | 952 | AAAAATTTAGTTGGTTATTGGTTACTGTA |
| | TGTATAATCTTACGGTAACCAATAACCAA | | ACAAACGATACCAATCCCCCAACCTCCAA |
| | CTTTAAAACT | | GTGGATAT |
| 622 | TTTAAATTTTGTCCTTTCTTCCCGCTATA | 953 | TTTTTATTTTTATCCCCTAATTATACATG |
| | CCCGCTTCCTCATATGTCAATAAGGATAA | | GGATTGGCATTGTAAAAGATAAATAGTTC |
| | AAATATTATT | | GCCCACTC |
| 623 | ATGGCTGTTGCGTTGATAGCGCCAAGCGT | 954 | GTTTTTTTGTTTGCGTTAAATGGAATTAT |
| | TACTAGTAGGACAGTTCCTAAAAGTGGCT | | CCAGTACGGCATATGCAGTAGAAACAACG |
| | AATTTTTTGT | | AGTCAACA |
| 624 | CCAAATATTAAATTCTGCAGTAGGCGTCC | 955 | AAAGTTTAGATGGGGTTTGTGGGTAGAGC |
| | AATTTCCGAATAACACACCAAAACCCCCA | | CTCCCAAAGGTTCCTCCACCCATAATTGT |
| | CATATGCCAC | | TATAGAAT |
| 625 | CATTTTTACCTTGCTCTTCTCTCGAATTT | 956 | AGTTTTATTTTTGTCTGTATAGGCTGTCC |
| | CAGCATCTGCGGTATGCTTATAGGGACAA | | GCATCTGCATGGCGCATAACATATTTATG |
| | AAATTATAAA | | CGCTACAG |
| 626 | TTTGCGAGACTACGGATCTGGATCTCGTC | 957 | GCTAACAGATCGGCATATGAGTGCTATCT |
| | CCACTGCTGGCAGTGAACTGTACTCAGAC | | ACTGCTGGCGCGGTCCCGCGATATCGCGC |
| | GCAAATAAGCA | | CGCAGGTAC |
| 627 | AGAAAAGCACGCTGATAATCAGCAAGACC | 958 | AATTGGAAAATATAAATAATTTTAGTAAC |
| | ACCAACATTTCAATCAAGGATAGTAAAAC | | CTACATTTCCACAAGTGTAAAAGCTTTAA |
| | TCTCACTCTT | | CCTTCGCT |
| 628 | ACACCAGAAATCAAGGAGTCTTACCAGTA | 959 | TTTTATCAAAAATTTTACTATCCTTGATT |
| | TGGAAATGTAGGTTACTAAAATTATTTAT | | GAGATGAAAATACAAGCTTCTTTACCAGT |
| | ATTTTCCACTT | | ATGATTCCG |
| 629 | ATGTACGAGTACTTTAGAGGGTATACAGC | 960 | TTATTTTATTATGGAAGTTTGTACACTTA |
| | CGTGGTTGCAAGACTGTACATACTTCCAT | | ACATTTATGCATGTGCCGCCAAAGTTGTC |
| | AGTTTATTAA | | TGAGGATT |
| 630 | AACAATCTGCAAACATGTATGGCGGTACA | 961 | ATTAATTTTGTACGGAAGTAGATACTATC |
| | TGTATCAATATAGAACGTTTATAGTTCCA | | TTTCAACATTGGTTGTATTCCTACAAAGA |
| | TACAAAAATA | | CACTCATT |
| 631 | TGTAACACTTCATTTTTGACGTTCAGAAA | 962 | TAAAATAGTATGTATTTATGTAAGTTTAA |
| | CAGCACGACCAACCTTACATAAATGGTAA | | CCACGACGAAATGTTCCTGGTTCAATGAC |
| | CTATTATATAT | | GACATATCT |
| 632 | GCTTCTGGACGCGGGTTCGATTCCCGCCG | 963 | CCCGACAGTTGATGACAGGGTGCGACCCC |
| | CCTCCACCAATATCCGAACCCTAACCGCT | | ACCACCACCCAACACCCCGGAAAGCCCTT |
| | CTCGGTTGGG | | GTTTTACA |
| 633 | GCTTCTGGACGCGGGTTCGATTCCCGCCG | 964 | CCCGACAGTTGATGACAGGGTGCGACCCC |
| | CCTCCACCAATATCCGAACCCTAACCGCT | | ACCACCACCCAACACCCCGGAAAGCCCTT |
| | CTCGGTTGGG | | GTTTTACA |
| 634 | GTAACACCAATTAAGTGTTTAGTTCCCTC | 965 | TATTTATAATTTTAGTTTCTCGATTCGTC |
| | TTTGCGTCCAGAGAGAGAAATTGAGGTAC | | TCCGTCCCTCATAGCTTGATCCGAAAAAG |
| | TAAACAACGTA | | TTACAGCTG |
| 635 | ACCGTAAAATAACATTTCTGTTTTTCCAG | 966 | GTAATTATTTTATGTATTCATTTCCGGCT |
| | CCCCGCAAGTAGCTAGTCTTGAATACCGA | | ATTCACACAGCCCAAATAAAAAAAGATTT |
| | AAAAAAATTC | | TTTCTGCT |
| 636 | GAATGATGCGTTGGGGCTTAATGGAGTAA | 967 | TATATTGTCATCACCCTGTTGGCGTCAAC |
| | ATCTAATTACACCAACAAGGTGACGACAA | | CTAATGCGCCTAATGGCTACAAAAGACAT |
| | AGCGCGAACG | | CTACTTTG |
| 637 | GAAACTATGGGGATTATAGCGTTTGAGGG | 968 | GAATAACTTTTTGCCGTATTGACATACCG |
| | AGCAAGTGCGGTGTATAATTAAGGCATAA | | CAAGTGCGGTTGGTAAGAGTAGCACGTGT |
| | AATAAAAAACG | | CGTGAATTA |
| 638 | TTCGGACGCGGGTTCAACTCCCGCCAGCT | 969 | GAATGAATAGCTAATTACAGGGACGCCAG |
| | CCACCAAATAAAACAAGGGGTTACGTGAA | | CCCAAATATTGATGTACTGAAGTTCAGTA |
| | AACGTAGCCCC | | AAGTCTACT |
| 639 | AATTTTTAAAAAAAGTCGACAAGCATTTA | 970 | TAATAGAAAGAAAAATATATTTATTATAT |
| | CTCTAATTGAAACGGCTTATAGTCATTAT | | CTAATTGAAGCAGCAATTGTGCTTTTCAT |
| | GTTTATTTTG | | TATTAGTT |
| 640 | AGAGAAGTTGCCGGAAGCATGGTTCTAGT | 971 | TAGATAGAGTTTATGGATTATAAGAGGTT |
| | TTCTTTGGGCAAAACCTCTTGAAATACAT | | TATTGGAAGAAAAGAAGGAACGAAGGAGT |
| | AAAAAGAGTT | | TAACGCGT |
| 641 | CACCTGGCGTGGCGAAGTGCGCAGTCTGG | 972 | AAGAGATTCACCAAGACTTTTAGATTGAC |
| | AAGCACTAGTACGTTGGCAGTCACCTGAA | | CACCTAAATAGCTGCGCGGAATAGTAGAT |
| | CGTGGGTTGAT | | CACTTTGAG |
| 642 | ATAACGCATACATTGTTGTTGTTTTTCCA | 973 | ATCAATAACGGTTGTATTTGTAGAACTTG |
| | GATCCAGTTTTTTTAGTAACATAAATACA | | ACCAGTTGGTCCTGTAAATATAAGCAATC |
| | ACTCCGAATA | | CATGTGAG |
| 643 | TATGTTCAGGTTTGATCATTTTCCAAAAA | 974 | ACTCAAATGACATCAATTCTGTCCTCTCA |
| | CGTATCATGTGGAGTGTGTTGTCTTGATG | | AGACAAAGCGTGTGTGTTCAACGTTTTTT |
| | TCAAGGGTGG | | TCTTTTCC |
| 644 | TATGTTCAGGTTTGATCATTTTCCAAAAA | 975 | ACTCAAATGACATCAATTCTGTCCTCTCA |
| | CGTATCATGTGGAGTGTGTTGTCTTGATG | | AGACAAAGCGTGTGTGTTCAACGTTTTTT |
| | TCAAGGGTGG | | TCTTTTCC |
| 645 | TATGCAACCCGTCGATATGTTCCCGCAAA | 976 | ATAGTAGGAAGATACTAAGTAGACAGTCA |
| | CAGCTCACATCGAGTGTGTAGGACTGCTT | | ACGCACGTGGAAACCGTAGTACTCTTGCA |
| | ACACGTGTGGA | | GTTAAAAGA |
| 646 | TAACACCAATTAAGTGTTTAGTTCCCTCT | 977 | GTCTTTATTTTTGGTATCCCGTTTCTTCT |
| | TTGCGTCCAACGAGAGAAATCGAGGTACT | | CCCTCCCTCATAGCTTGAACCGAAAAAGT |
| | AAACAAGCTAA | | TACAGCTGG |
| 647 | GTAACACCAATTAAGTGTTTAGTTCCCTC | 978 | ATTATTATGGATTAGTATCTCATTTATTC |
| | TTTGCGTCCAGCGAGAGATAACGAGGTAC | | TCCGTCCCTCATAGCTTGATCCGAAAAAG |
| | TAAATAATCTA | | TTACAGCTG |
| 648 | GCTGGTGGTGGATATCGGCGGTGGTACGA | 979 | TCCATTAACTGTGGTGTACATCATAACAT |
| | CTGACTGTTCGTAGTCATGCAATAATGTA | | AACTGTTCATTGCTGCTGATGGGGCCGCA |
| | CACCGCAGTAA | | GTGGCGTTC |
| 649 | TATGCAACCAGTCGATATGTTCCCGCAAA | 980 | ATAGTAGGAAGATACAGAGTGTACTCTCA |
| | CAGCTCACATCGAGTGTGTAGGACTGCTT | | ACGCATGTAGAGACCGTAGTACTTTTGCA |
| | ACACGTGTGG | | GTTAAAAG |
| 650 | AACCAGCTGTAACTTTTTCGGATCAAGCT | 981 | TTAGCTTGTTTAGTACCTCGATTTCTCTC |
| | ATGAGGGAGGGAGAAGAAACGGGATACCA | | GTTGGACGCAAAGAGGGAACTAAACATTT |
| | AAAATAAAGAC | | AATTGGTGT |
| 651 | AACCAGCTGTAACTTTTTCGGATCAAGTT | 982 | TTAGATTATTTAGTACCTCGTTATCTCTC |
| | ATGATGGAAGAAGAAGAAACGAGAAACTA | | GCTGGACGTAAAGAGGGAACAAAGCACCT |
| | AAATTATAAAT | | AATAGGTGT |
| 652 | TAACACCAATTAAGTGTTTAGTTCCCTCT | 983 | GTCTTTATTTTTGGTATCCCGTTTCTTCT |
| | TTGCGTCCAACGAGAGATAACGAGATACT | | CCCTCCCTCATAGCTTGAACCGAAAAAGT |
| | AAACAATCTAA | | TACAGCTGG |
| 653 | ATAATCATCAAAGATTTTAGGATTATCAA | 984 | TACTTTAATTTTGGGTTAATGGTCCATTT |
| | ATTCACTAGTAAATGTATTATTAACCCAA | | CCTCTATGATACGCCCTTCCGAAAGCTGA |
| | AAAAAGAGTCT | | TACTAACGA |
| 654 | CATCTTTACTTTGCTCTTTTCTCGAATTT | 985 | AGTTTTATTTTTGTCTATATAGGCTGTCG |
| | CAGCATCTGCGGTATGCTTATAGGGACAA | | GCATCTGCGTGTCTCATAACGTATTTATG |
| | AAATTATAAA | | CGCTACAG |
| 655 | CTGTTTCAACAAATGATGCTCTTGGCCTT | 986 | AAAAATAAATATCTTTGTCGCCATCGTGT |
| | AATGGTGTAAACCTAATTACACCAACAAG | | TGGTGTAAACCTTATGCGTTTAATGGCGA |
| | GTGACAACAAA | | CAAAACATA |
| 656 | AGCTAAGTGTCCTAATTGGCCCCCGATCC | 987 | TACATAATTTCGTATATTAGGTATAACCA |
| | CGGTTTCAATTGGAAATACCTAATATACG | | GTTTCAATAGTTTGGGGAATCTTTGTAAG |
| | AAAAAGGTGT | | TGGTAAGC |
| 657 | CGGCCTTCCACTTACAAAAATTCCGCAGA | 988 | CGCCTTTTTTCGTATATTAGGTATTTCCA |
| | CAATTGAAACTGGTTATACCTAATATACG | | ATTGAAACCGGGATCGGGGGCCAATTAGG |
| | AAAATATGCA | | ACACTTAG |
| 658 | GTAGATGTTTTTTGTTGCCATTAGGCGCA | 989 | CGCTTTGTTGTCACCTTGTTGGTGTAATT |
| | TGAGGTTGTTACCAACAGGGTGATAACAA | | AGATTTACTCCATTAAGCCCTAAAGCATC |
| | AGCTAATGAA | | ATTCGTCG |
| 659 | AATATGTTTTGTCGCCATTAAACGCATAA | 990 | TTTGTCGTCACCTTGTTGGTGTAATTAGG |
| | GGTTTACACCAACATGATGACAACGAAGA | | TTTACACCATTAAGGCCAAGAGCATCATT |
| | TATTTACTTTT | | TGTTGAAAC |
| 660 | AATATGTTTTGTCGCCATTAAACGCATAA | 991 | TTTGTCGTCATCTTGTTGGTGTAATTAGG |
| | GGTTTACACCAACTTGATGACGACAAAAA | | TTTACACCATTAAGGCCAAGAGCATCATT |
| | TATTTATTTTT | | TGTTGAAAC |
| 661 | CGTCGTTAGTATCAGCTTTCGGAAGGGCG | 992 | AGACTCTTTTTTTGGGTTAATAAAACATT |
| | TATCATAGAGGAAATGGACCATTAACCTA | | TACTAGTGAATTTGATAATCCTAAAATCT |
| | AAATTAAAGTA | | TTGATGATT |
| 662 | GCGCGTGATATTGCGACGTATTTTAATCA | 993 | ACAATACATTTTACTTCAATGTATAGGTA |
| | TACATTCGGCACAGCGAGTTTATCTATAA | | CATTCGGCACGACATTTACACTTCCGAAG |
| | GTTGAAGTAA | | TATGTCAT |
| 663 | GTTTTTTGTTGCCATTAGGCGCATGAGGT | 994 | GTCGTCACCTTGTTGGTGTAATTAGGTTG |
| | TGACGCCAACAGGGTGATGACAATATAAA | | ACTCCATTAAGCCCTAGAGCATCATTCGT |
| | CATTTCTTTTT | | CGAAACAGC |
| 664 | ATTGATTCTACAACAGAAGTTGGCATACT | 995 | CGCTCCTTTAATTTTGCTTAAAGGAGCAA |
| | AGAAACTAGTATCTTATTTATCTTAAGCT | | AGACTAGTACTTTAAGAGCACCAAAAATA |
| | AAAATTAAAAT | | AATAATGTA |
| 665 | CATCTTTACTTTGCTCTTCTCTCGAATTT | 996 | AGTTTAATTTTTGTCTATATTGGCTGTCT |
| | CAGCATCTGCGGTATACTTATAGGGACAA | | GCATCTGCATGGCGCATCACATATTTATG |
| | AAATTATAAA | | CGCTACAG |
| 666 | AAAATTAACAAGCTAATAATGAACAAGAC | 997 | TTTTATACCTTTTTGAATATATTTAGAGA |
| | AATCGTCATTTCAATAGCACTCCCCAAAT | | TCGTCATTTCCACCAGGGTAAAGCCCTTG |
| | CTTTTTAATAG | | GCCACCCGT |
| 667 | TTTGTTGACTCGTTGTTTCTACTGCATAT | 998 | ACAAAAAATTAGCCACTTTTAGGAACTGT |
| | GCCGTACTGGATAATTCCATTTAACGCAA | | CCTACTAGTAACGCTTGGCGCTATCAACG |
| | ACAAAAAAAC | | CAACAGCC |
| 668 | TAACACCAATTAAGTGTTTAGTTCCCTCT | 999 | TGTTCTTTTTTTGGTATCTCGTTTCTTCT |
| | TTGCGTCCAACGAGAGAAAACGAGGTACT | | TCTTCCCTCATAGCTTGATCCGAAAAAGT |
| | AAATAAACTAA | | TACAGCTGG |
| 669 | GTCTTCTGGACCATGATGCGCCACTTCCG | 1000 | TGTATCTTGATGTACAACATTGCTCTTTA |
| | AAATTTCAAATACAGAATAATGTTGCATA | | TTTTCAAAAAGATCAGTGGTCAAACGGCT |
| | AAATAGCCCTG | | CATTAATTT |
| 670 | TAACACCAATTAAGTGTTTAGTTCCCTCT | 1001 | ATGTTCTTTTTTGGTATCTCGTTTCTTCT |
| | TTGCGTCCAGCGAGAGATAACGAGGTACT | | TCTTCCCTCATAGCTTGATCCGAAAAAGT |
| | AAATAATCTAA | | TACAGCTGG |
| 671 | CGCGACACCAGCCTCGTCGTGGTCCCGCA | 1002 | GGTTTTCTTTGCCCCTTTGCGCGCACAGT |
| | GTTCCACGTATGTGCGCGCAAAGGGGGAA | | CCCACGTCAACGCCTGGGGCCTGCCGCAC |
| | GGAGGCGGCC | | GCGGTGTT |
| 672 | GTGTCGGCAGCCCTGCAGGTCGGATATCG | 1003 | CTGCATCTACCATGTTCTACAATCTACCA |
| | CAGCATCGACACTTCATTGGTAGGACTTG | | GCATCGACACCGCCAAGATCTACGACAAC |
| | GTAGAACGGT | | GAGGCGGG |
| 673 | TCCGCAGCAATATCTTCATACAAATCGGC | 1004 | GCGCATTTAGTTTGTGTTTTTAAAAGCAA |
| | AATAGGATCTCCTTTTGCTTTTAAAGACA | | TAGGATCTCCTTTTGCCTGGATATAAGTG |
| | TAACAAATAGT | | GCAGTGAAT |
| 674 | TATCTTTTAACTGCAAGAGTACTACGGTT | 1005 | TCTTGGCGAGTGAGCAGACCTATACACTC |
| | TCCACGTGCGTTGACTGTCTACTTAGTAT | | GATGTGAGCTGTTTGCGGGAACATATCGA |
| | CTTCCTACTAT | | CGGGTTGCA |
| 675 | ACCAGCTGTAACTTTTTCGGATCAAGCTA | 1006 | TACGTTGTTTAGTACCTCAATTTCTCTCT |
| | TGAGGGACGGAGACGAATCGAGAAACTAA | | CTGGACGCAAAGAGGGAACTAAACACTTA |
| | AATTATAAATA | | ATTGGTGTT |
| 676 | CATTTTTACCTTGCTCTTCTCTCGAATTT | 1007 | AGTTTTATTTTTGTCTGTATAGGCTGTCC |
| | CAGCATCTGCGGTATGCTTATAGGGACAA | | GCATCTGCATGGCGCATAACATATTTATG |
| | AAATTATAAA | | CGCTACAG |
| 677 | ACCAGCTGTAACTTTTTCGGATCAAGCTA | 1008 | TAGATTATTTAGTACCTCGTTATCTCTCG |
| | TGAGGGACGGAGACGAATCGAGAAACTAA | | CTGGACGCAAAGAGGGAACTAAACACTTA |
| | AATTATAAATA | | ATTGGTGTT |
| 678 | TATGCAACCCGTCGATATGTTCCCGCAAA | 1009 | ATAGTAGGAAGATACTAAGTAGACAGTCA |
| | CAGCTCACATCGAGTGTGTAGGTCTGCTT | | ATGCACGTGGAAACTGTAGTACTCTTGCA |
| | ACTCGTGTAGA | | GTTAAAAGA |
| 679 | TCGTTTCAATATGTCCGTACATGGAATAA | 1010 | ATCATCCTTATACGTGTTTAGCTATGTAA |
| | TAAAGCACCAGTATTCTTGCCTTAACACT | | AAGCACCAGAACTTTAGCCATTTCTAACC |
| | CATGGTATTC | | ACTCCTCG |
| 680 | CGAACATCTATAAATTCTGTATTGGTAGA | 1011 | GGTTTTTTTGTGTGTGGTTTTGTATGTTA |
| | AACATCACAATCAAAATGCTAATACCACA | | AATCACAGGTGCTTTCCCTCCTGGTGAAC |
| | CACTACAATA | | AGTACAAC |
| 681 | ATAGTATTAGCTGGCGGATGTGCAACTGG | 1012 | ATTACAATATTACTTTATTTAGTCTATCT |
| | CACATGGTGGAACTGGACTGAATTAAGTC | | TTAGGTATCGAGCTGGGGAAGGATTAATT |
| | AAAATATAAAC | | GGTAGTTGG |
| 682 | CGACAAGGACACCACGCTCGTCGTGGTCC | 1013 | CACCTTTTTTATTTGCCCCTTTAGGCGCA |
| | CTCAATTTCACGTCTGTGAGCCTAAAGGG | | CTGTTCCACGTGAACGCCTGGGGCCTGCC |
| | GCATCCCCAC | | GCACGCCA |
| 683 | GACGACGTCAAATGAGAAATCTGTTACAC | 1014 | TTTTTACAAAGAGGTATTTAGATACATGA |
| | GTGTAACAATGCCTGTATCTAAATACCTC | | GCTACATTAGCAGTTAACCGCCGTTTTAA |
| | TAAAGAAAGAC | | ATCGCAAAA |
| 684 | CTGTGCCGCCCGAGTGATCTGCGTGCACA | 1015 | AAAGTTTTTTTAGACGTACTAACCAATAT |
| | ATCATCCCAGCGGAAAGTATCAGTTAGGC | | CATCCCAGCGGCAGTCCCCAACCTTCGCA |
| | ACATAAATTAG | | GGCGGATAT |
| 685 | ATGGCTGTTGCGTTGATAGCGCCAAGCGT | 1016 | GGTTTTTTGTTTGCGTTAAATGGAATTAT |
| | TACTAGTAGGACAGTTCCTAAAAGTGGCT | | CCAGTACGGCATATGCAGTAGAAACAACG |
| | AATTTTTTGT | | AGTCAACA |
| 686 | GAATGATGCGTTGGGGCTTAATGGAGTAA | 1017 | TATATTGTCATCACCCTGTTGGCGTCAAC |
| | ATCTAATTACACCAACAAGGTGACGACAA | | CTAATGCGCCTAATGGCTACAAAAGACAT |
| | AGCACGAACG | | CTACTTTG |
| 687 | GTCTTCTGGACCATGATGCGCCACTTCCG | 1018 | TGTATCTTGATGTACAACATTGCTCTTTA |
| | AAATTTCAAATACAGATTAATGTTGTATA | | TTTTCAAAAAGATCAGTGGTCAAACGGCT |
| | AAGTAACCCTG | | CATTAATTT |
| 688 | ATAGAAATAGACCTTTCCACTGGCCAAGG | 1019 | AATTATTACTTGTGTTTTTGTAGTGGTTG |
| | AGCTGATAAAACTATTACAAATACACAAG | | CTGATAAAACCATGCAACAAGTTTTAAGT |
| | TATAGAAATAG | | AAAAGTGCA |
| 689 | TTGATATGATATTTTATAACGGTTAATAT | 1020 | GGGAAAGTTTTGGGGAAGATTTTACATCA |
| | ATTTATAATAAATATCCTCCGGCATAGCC | | TCATAAAACAACGGGCGTGTTATACGCCC |
| | GGAGGTTTTT | | GTTTCAAT |
| 690 | AACGTTTGTAAAGGAGACTGATAATGGCA | 1021 | ATGGATAAAAAAATACAGCGTTTTTCATG |
| | TGTACAACTATACTAGTTGTAGTGCCTAA | | TACAACTATACTCGTCGGTAAAAAGGCAT |
| | ATAATGCTTT | | CTTATGAT |
| 691 | GATAGTGATCGAATATATTCATGGTATGC | 1022 | TAAAATGTTCCCATTGATTGTGGTGTGTG |
| | CGTCCTTTCGTATACTATGGGAACATTTT | | TCCTTTCGTTTTTTAGCACAGGTTAAGAG |
| | GATTTAATAC | | CCGTTCAT |
| 692 | CCCGAAGGATGCTCCCCGCTCCACCACCG | 1023 | TGGGGTCTTGCATCCAGCGTGAATGGTTG |
| | TTTATGAAACTTTCATGCCACGCTGGATA | | TGCGACCCGACCTGTGGATCTGGTTCGCT |
| | CAAACGCGCG | | GTTGATCA |
| 693 | AATGTTTATCGTTACTTTTGGAGGTACGG | 1024 | TTTTTTTACGTGAATGTTTTGTAACTACT |
| | GTGCAACCTACCTCGTAACACACCATTCA | | ACGACATTGGTCGTCCCGTTCATGTTTAT |
| | TCAAAATCTA | | GTGGATGA |
| 694 | TAACTCACGACACGTTGTGCTCTTACCAA | 1025 | GTTTTTATTTTATGCCTTAATTATACACC |
| | CCGCACTTGCAGTATGTCAATATGGCAAA | | GCACTTGCTCCCTCAAACGCTATAATCCC |
| | AAGCTATTCT | | CATAGTTT |
| 695 | ACAATCATCAGATAACTATGGCGGCACGT | 1026 | TTAATTTAGTATGGAAGTATGCACAATTA |
| | GCATTAATGTTTAGTGTGTATACTTCCAT | | ACCAACCACGGTTGTATCCCGTCTAAAGT |
| | AAAAATTAAC | | ACTCGTAC |
| 696 | TATGCAACCAGTCGATATGTTCCCGCAAA | 1027 | ATAGTAGGAAGATACTAAGTAGACAGTCA |
| | CAGCTCACATCGAGTGTGTAGGACTGCTT | | ACGCATGTAGAGACCGTAGTACTTTTGCA |
| | ACACGTGTGG | | GTTAAAAG |
| 697 | GCAACCGGCATCAATGTAATACCGATAAT | 1028 | CAAATAATGTAGTACCCAAATTATGTTTC |
| | CGTAACAAGCAACCTTAATCGGGTACTAC | | ACACAACAGAGCCTGTCACGACCGGCGGA |
| | TTAATATCTA | | AAAAACGA |
| 698 | AAGAACACTAATAATCAGCAAAACAACTA | 1029 | TGGAAAATTTGATAAATTTGGTTACGTTC |
| | GCATTTCAATCAAGGATAGTGAAATTATT | | ATTTCAATCAGCGTAAAAGCTTTTACTTT |
| | GCTTTTTCGAA | | GAGTGTACG |
| 699 | GAGAGAGTAGAGTGTTGTTGTCTTGCCAG | 1030 | CTTGTTTTATTAATATTTACGTAACGTTA |
| | ACCCAGTTGGTAGCGTTACGTAAATATAA | | TCAGTTGGACCGGTCAGAATTATTAATCC |
| | CTAATTATTTA | | GTGTGCATG |
| 700 | CTTGTAAAACAAGGGCTTTCCGGGGTATT | 1031 | CCCAACCGAGAGCGGTTAGGGTTCGGATA |
| | GGGTGGTGGTGGGGTCGCACCCTTGTATG | | TTGGTGGAGGCGGCGGGAATCGAACCCGC |
| | AAACTGACCT | | GTCCAGAA |
| 701 | CTTGTAAAACAAGGGCTTTCCGGGGTATT | 1032 | CCCAACCGAGAGCGGTTAGGGTTCGGATA |
| | GGGTGGTGGTGGGGTCGCACCCTTGTATG | | TTGGTGGAGGCGGCGGGAATCGAACCCGC |
| | AAACTGACCT | | GTCCAGAA |
| 702 | CACTCCCAAAGTCGGCTTCGTCAGTCTTG | 1033 | CTCCCAGTGTAGGATTTATATCGCTAGGG |
| | GATGCCCCAACGAATAGAAAAGTAAACCA | | TGCCCCAAGGCGCTGGTCGACTCCGAGCG |
| | GTTTTCAGCG | | CATCCTCA |
| 703 | CACTCCCAAAGTCGGCTTCGTCAGTCTTG | 1034 | CCCCTAGTATAGGATGGGTTTCGTTAGGG |
| | GATGCCCCAACGAATAGAAAAGTAAACCA | | TGCCCCAAGGCGCTGGTCGACTCCGAGCG |
| | GCTTTCAGCG | | CATCCTCA |
| 704 | ATGATCTGCTCCGAATCGACGAGTGCCTT | 1035 | AGCGATGAGTATACTTTTGCTATCCTACG |
| | GGGGCACCCAAGCGACACCATTCCTATAC | | GGCACCCAAGGGATACAAAGCCCACACGC |
| | TATACGGCTTC | | GGATTGTGG |
| 705 | GTCTTCTGGACCATGATGCGCCACTTCCG | 1036 | TGTATCTTGATGTACAACATTGCTCTTTA |
| | AAATTTCAAATACAGAATAATGTTGCATA | | TTTTCAAAAAGATCAGTGGTCAAACGGCT |
| | TAATATTACTA | | CATTAATTT |
| 706 | AAAGCTAAGGTTAAAGCTTTTACATTGAT | 1037 | AAGAGTGAGAGTTTTACTATCCTTGATTG |
| | TGAAATGTAGGTTACTAAAATTATTTATA | | AAATGTTGGTGGTCTTGCTGATTATCAGC |
| | TTTTCCAATT | | GTGCTTTT |
| 707 | TAGATACACCTGCAATTTGTTGTAATGGC | 1038 | CTTCTAATTTTTGTTTGTATAAGCATAAC |
| | ACTTATTTGAGTGTGTGACGCTTATTACA | | ACATTTGTATGATTATCAGGCAAAAAAGG |
| | ACATTTTCACC | | TTTTAGAAT |
| 708 | TCGTACGCCGGGGAGACGACGTTCGCCGC | 1039 | AGCTCGGGTTCTTCGTGTTTTGCCACGTA |
| | GATGTTGACCGACAGACACGGCAAAACAC | | TGTTGACCGAGAGCGTGGCGACGAGGACG |
| | GCAGCGCCTAT | | GTCACCAGG |
| 709 | GGATTTCGTTGCACTGATGGGCGGTACTG | 1040 | TCTTTTTTTATGTATGGTTTGTAACAATA |
| | GCGCGACCTACAATGTGCTAAACCATACA | | TCCACTTTACTCGTTCCTTATTTATTTAT |
| | TGTTAAAAAT | | ATTTCTTT |
| 710 | AGTACAACCAGTCGATTTATTCCCACAAA | 1041 | ATAGTAGGAAGATACAGAGTGTACTCTCA |
| | CACATCACATCGAGTGTGTAGGACTGCTT | | ACGCATGTGGAATTAGTGGCGCTATTAGC |
| | ACACGTGTGG | | ACCTAAGG |
| 711 | AGTACAACCAGTCGATTTATTCCCACAAA | 1042 | ATAGTAGGAAGATACAGAGTGTACTCTCA |
| | CACATCACATCGAGTGTGTAGGACTGCTT | | ACGCATGTGGAATTAGTGGCGCTATTAGC |
| | ACACGTGTGG | | ACCTAAGG |
| 712 | ACATAAAAATATAGATTTTCCAGGGCATA | 1043 | CGAAATATCGCAATTACATAAAGCATGTA |
| | ATCATGCATGGTTTATAGTATTGCAACCA | | CATGCATGGCTATATGATGTGAATAAAAT |
| | TTCTACCAAAT | | AGAACCCGA |
| 713 | GTCTTCTGGACCATGATGCGCCACTTCCG | 1044 | TGTATCTTGATGTACAACATTGCTCTTTA |
| | AAATTTCAAATACAGAATAATGTTGCATA | | TTTTCAAAAAGATCAGTGGTCAAACGGCT |
| | TAATATTACTA | | CATTAATTT |
| 714 | GGTTAAGTGTATGGATATGTTCCCAAATA | 1045 | TGTTGAATAGGTTGGTCATTGGAGAACCG |
| | CGCCACACGTTGAGAGCGTAGTATTGTTG | | AGCCATTGTGAGACTGTAGTTAAACTTAT |
| | ACTAAAGCAC | | TAGAGAAT |
| 715 | GGTTAAGTGTATGGATATGTTCCCAAATA | 1046 | TGTTGAATAGGTTGGTCATTGGAGAACCG |
| | CGCCACACGTTGAGAGCGTAGTATTGTTG | | AGCCATTGTGAGACTGTAGTTAAACTTAT |
| | ACTAAAGCAC | | TAGAGAAT |
| 716 | AAAGCGAATGGCAAGCTCAGGCCACTCGG | 1047 | TTGAGCACTTGTGCAGTTCGCGTTGACCG |
| | CATTCCGACGGTGACTTCATAATGCACCT | | TCCCGAGCCTGCGGGATCGGATCGTGCAG |
| | CTCACAGTTG | | CGGGCTAT |
| 717 | TAAGAAGAAAGACTCTTTTTTTATTTGGG | 1048 | TGAATTTTTTTCGGTATTCAAGACCAGCT |
| | CTGTGTGAATAGCCCGAAATGAATACATA | | ACTTGCGGGGCTGGAAAAACTGAAATGCT |
| | AAAAGATAAC | | ATTTTACG |
| 718 | GACTGCGCCTCTAAAGATTTCCCTTGGAT | 1049 | CGTTTATAGTGTTTTAGGTGGTTGGCACC |
| | GAGCTACCGACATAGCTATATCAACCCTC | | CCTACCGATTGACTTAATCCCCCAACAAA |
| | AATAAATTTAT | | AGTCGTTTC |
| 719 | TCACACAATTGACCAACTATTAGTAACTC | 1050 | CTAATAATTGTATCAAATATGGAACGCAT |
| | ACGCAGAAGTGTGAGTTCTGAAATTGATA | | ACCGATACTGATCATATGGGGGATATCGA |
| | CAATACAACT | | AGTGGTTG |
| 720 | TCACACAATTGACCAACTATTAGTAACTC | 1051 | CTAATAATTGTATCAAATATGGAACGCAT |
| | ACGCAGAAGTGTGAGTTCTGAAATTGATA | | ACCGATACTGATCATATGGGGGATATCGA |
| | CAATACAACT | | AGTGGTTG |
| 721 | CCATCATAAGATGCCTTTTTACCGACGAG | 1052 | AAAGCATTATTTAGGCACTACAACTAGTA |
| | TATAGTTGTACATGAAAAACGCTGTATTT | | TAGTTGTACATGCCATTATCGGTCTCCTT |
| | TTTTATCCAT | | TACAAACG |
| 722 | CCATCATAAGATGCCTTTTTACCGACGAG | 1053 | AAAGCATTATTTAGGCACTACAACTAGTA |
| | TATAGTTGTACATGAAAAACGCTGTATTT | | TAGTTGTACATGCCATTATCAGTCTCCTT |
| | TTTTATCCAT | | TACAAACG |
| 723 | CCATCATAAGATGCCTTTTTACCGACGAG | 1054 | AAAGCATTATTTAGGCACTACAACTAGTA |
| | TATAGTTGTACATGAAAAACGCTGTATTT | | TAGTTGTACATGCCATTATCAGTCTCCTT |
| | TTTTATCCAT | | TACAAACG |
| 724 | ACGTTTGTAAAGGAGACTGATAATGGCAT | 1055 | TGGATAAAAAAATACAGCGTTTTTCATGT |
| | GTACAACTATACTCGTTGTAGTGCCTAAA | | ACAACTATACTCGTCGGTAAAAAGGCATC |
| | TAATGCTTTTA | | TTATGATGG |
| 725 | ACCTCCGCGCGGTCGCGCCGCGTGCGGTC | 1056 | AACGATGCTCGCGAGTCCTTTAGAGACAC |
| | GTTCACCCACGTCAGTGGATCTAAAGGAC | | TGACCCAGGGGTCCGGCAGGAACAGCCGC |
| | CACATCGGAGC | | CAGTTGACG |
| 726 | ACAATCAACAAAGATGTATGGTGGTACAT | 1057 | TAACTTATGTACGGAAGTATAGACACTC |
| | GCATTAATATTTAATGTGTATACTTCCGT | | GATTAATATCGGATGTATACCTACTAAA |
| | AAAAATAACC | | ACATTAATTC |

Alternative Recognition Sites

| 1720 | AAAATATTTAGTTTTCTTTGGAGGAGCTG | 1776 | TTTTTAAATTTTGGTAATTAATGGAGTG |
|---|---|---|---|
| | GGACATCAACTGAAATTACTTCTATAAAC | | AACATCAACGGATAGCGGTGTTAAAGAT |
| | TACCAAAATA | | TTTCGGGGAA |
| 1721 | AACAGTTCCTTTTTCAATGTTACTGTATC | 1777 | TTATTTATAGACTTTTTGTCAAATATAG |
| | CTGATGTGTACTTTACAAAAACACTATTT | | TGATGTGTACCTATAGCCCATCCGTCGC |
| | TATATAAATA | | GCAATGAAAG |
| 1722 | AACCAGCTGTAACTTTTTCGGTTCAAGCT | 1778 | TTAGCTTATTTAGTACCTCGTTTTCTCT |
| | ATGAGGGAGGGAGAAGAAACGGGATACCA | | CGTTGGACGCAAAGAGGGAACTAAACAC |
| | AAAATAAAGAC | | TTAATTGGTGT |
| 1723 | AAGTGTAATATGTTTGGGTATGGGGAAGT | 1779 | GAAAAAAAGTGTACATGGTAGAGAGTTA |
| | GAATCAGTTTAATACTCCACCATGTACAC | | AACCAGTACAATCGCCACAGTACACTTA |
| | GAAGTGAAAA | | TGTCAGCCTA |
| 1724 | AATGAGCTAAAAGCTGTGGCCCAGTCATC | 1780 | TTTATTTAATGTAGTTAGGTTGTGTTTA |
| | AATTGACCAAACACTATATAACTACAATA | | ATTGACCAAACCATGGTGTTTGAAATGC |
| | AAAGAGCACA | | ACTGCCGCCA |
| 1725 | ACAATCAACAAAGATGTATGGCGGTACAT | 1781 | TAACTTATGTACGGAAGTATAGACACTT |
| | GCATTAATATTTAATGTGTATACTTCCGT | | GATTAATATCGGATGTATACCGACTAAA |
| | ATTTTTATAG | | ACATTAATTC |
| 1726 | ACAATCGTCAGATAATTTTGGCGGTACAT | 1782 | TTAATAAACTATGGAAGTATGTACAGTC |
| | GCATAAATGTTGAGTGAACAAACTTCCAT | | TTGCAATCACGGCTGTATCCCCTCTAAA |
| | AATAAAATAA | | GTGCTCGTGC |
| 1727 | ACCAGCTGTAACTTTTTCGGATCAAGCTA | 1783 | TAGATTATTTAGTACCTCGTTATCTCTC |
| | TGAGGGACGGAGACGAATCGAGAAACTAA | | GCTGGACGCAAAGAGGGAACTAAACACT |
| | AATTATAAATA | | TAATTGGTGTT |
| 1728 | ACCGTAAAATAGCATTTCAGTTTTTCCAG | 1784 | GTTATCTTTTTATGTATTCATTTCGGGC |
| | CCCCGCAAGTAGCTGGTCTTGAATACCGA | | TATTCACACAGCCCAAATAAAAAAAGAG |
| | AAAAAATTCA | | TCTTTCTTCT |
| 1729 | AGCAACGCCAGATAGAACAGCATGATCTT | 1785 | AGCATGGTTTGTATATTGGCTAACGTTC |
| | CGGGTTGCCGAGCGTTAGCCAATATACAT | | GGGTTGCCGAGCGTGACCAGCGTGCCGG |
| | ATTAACAGGGC | | CCGCGAACATG |
| 1730 | AGCTTTCATTGCGCGACGGATGGGCTATA | 1786 | TATTTATATAAAATAGTGTTTTTGTAAA |
| | GGTACACATCACCATATTTGACAAAAAAC | | GTACACATCAGGTTACAGTAACATTGAA |
| | CTATAAATAA | | AAAGGAACTG |
| 1731 | ATAATCATCAAAGATTTTAGGATTATCAA | 1787 | TACTTTAATTTTAGGTTAATGGTCCATT |
| | ATTCACTAGTAAATGTTTTATTAACCCAA | | TCCTCTATGATACGCCCTTCCGAAAGCT |
| | AAAAAGAGTCT | | GATACTAACGA |
| 1732 | ATAATCATCAAAGATTTTCGGATTATCAA | 1788 | TACTTTAATTTTAGGTTAATGGTCCATT |
| | ATTCACTAGTAAATGTTTAATTAACCCAA | | TCCTCTATGATATGCCCTGCTGAAAGCT |
| | AAAAAGAGTCT | | GATACTAACGA |
| 1733 | ATCTTTTAACTGCAAAAGTACTACGGTCT | 1789 | CCACACGTGTAAGCAGTCCTACACACTC |
| | CTACATGCGTTGAGAGTACACTCTGTATC | | GATGTGAGCTGTTTGCGGGAACATATCG |
| | TTCCTACTAT | | ACTGGTTGCA |
| 1734 | ATCTTTTAACTGCAAAAGTACTACGGTCT | 1790 | CCACACGTGTAAGCAGTCCTACACACTC |
| | CTACATGCGTTGAGAGTACACTCTGTATC | | GATGTGAGCTGTTTGCGGGAACATATCG |
| | TTCCTACTAT | | ACTGGTTGCA |
| 1735 | ATGAATTAATGTTTTAGTAGGTATACATC | 1791 | TATAAAAAATACGGAAGTATACACATTA |
| | CGATATTAATCAGGTGTCTATACTTCCGT | | AATATTAATGCATGTACCACCATACATC |
| | ACATACGTTA | | TTTGTTGATT |
| 1736 | ATGTACGAGTACTTTAGACGGGATACAAC | 1792 | GTATAAATATATGGAAGTACACACATTA |
| | CGTGGTTGCTCAATTGTGCATACTTCCAT | | TACATTAATGCACGTGCCGCCATAGTTA |
| | ACTAAATTAA | | TCTGATGATT |
| 1737 | ATTTAACATCAATGAACCTGAACCCATGG | 1793 | CACGGCATTGTATTAAACTCAGTAAGAT |
| | TTGGATCTATGTTCCTACTGATTTTGATA | | TATTTCAAAAACACTAAAGAATCGTCGT |
| | CAAAAGAAAA | | TCTTTTTGAT |
| 1738 | ATTTAACATCAATGAACCTGAACCCATGG | 1794 | CACGGCATTGTATTAAACTCAGTAAGAT |
| | TTGGATCTATGTTCCTACTGATTTTGATA | | TATTTCAAAAACACTAAAGAATCGTCGT |
| | CAAAAGAAAA | | TCTTTTTGAT |
| 1739 | ATTTATTTCGTTCCGTGTTAGGTAATATT | 1795 | GTAGGCTCTTTTTGGGTTAATATAACAC |
| | ACGAGTAGAGTCAATGTTCCTTTAACCCA | | TCACTAGCGAAGAAGGTCTGCCAAAAGA |
| | AAAATTAAAGG | | AAATTTAGATT |
| 1740 | CACTCCCAAAGTCGGCTTCGTCAGTCTTG | 1796 | CCCCTAGTATAGGATGGGTTTCGTTAGG |
| | GATGCCCCAACGAATAGAAAAGTAAACTA | | GTGCCCCAAGGCGCTGGTCGACTCCGAG |
| | GCTTTCAGCG | | CGCATCCTCA |
| 1741 | CACTCCCAAAGTCGGCTTCGTCAGTCTTG | 1797 | CCCCTAGTATAGGATGGGTTTCGTTAGG |
| | GATGCCCCAATGACTGCAAAAGTAAACTC | | GTGCCCCAAGGCGCTGGTCGACTCCGAG |
| | AATCTTTAAG | | CGCATCCTCA |
| 1742 | CCATCATAAGATGCCTTTTTACCGACAAG | 1798 | AAAGCATTATTTAGGCACTACAACTAGT |
| | TATAGTTGTACATGAAAAACGCTGTATTT | | ATAGTTGTACATGCCATTATCAGTCTCC |
| | TTTTATCCAT | | TTTACAAACG |
| 1743 | CCATCATAAGATGCCTTTTTACCGACGAG | 1799 | AAAGCATTATTTAGGCACTACAACTAGT |
| | TATAGTTGTACATGAAAAACGCTGTATTT | | ATAGTTGTACATGCCATTATCGGTCTCC |
| | TTTTATCCAT | | TTTACAAACG |
| 1744 | CCATCATAAGATGCCTTTTTACCGACGAG | 1800 | AAAGCATTATTTAGGCACTACAACTAGT |
| | TATAGTTGTACATGAAAAACGCTGTATTT | | ATAGTTGTACATGCCATTATCAGTCTCC |
| | TTTTATCCAT | | TTTACAAACG |
| 1745 | CTGAGTGGGCGAACTATTTATCTTTTACA | 1801 | AATAATATTTTTATCCTTATTGACATAT |
| | ATGCCAATCCCATGTATAATTAGGGGATA | | GAGGAAGCGGGTATAGCGGGAAGAAAGG |
| | AAAATAAAAA | | ACAAAATTTA |
| 1746 | GAAACTATGGGGATTATAGCGTTTGAGGG | 1802 | GAATAGCTTTTTGCCATATTGACATACT |
| | AGCAAGTGCGGTGTATAATTAAGGCATAA | | GCAAGTGCGGTTGGTAAGAGCACAACGT |
| | AATAAAAACTG | | GTCGTGAGTTA |
| 1747 | GAAGGGAATAATAGCTCTGTTTTGCCTGC | 1803 | GTGGAATTTTTAGTATTCATAACGGGCT |
| | TCCACAAACAACCAATCATGAATACTAAA | | ATTCAAACTGCCCAAATCAAATATTCCG |
| | ATTATCATAAA | | ACAGCCCTGGT |
| 1748 | GACCACAATCCGCGTGTGGGCTTTGTATC | 1804 | GAAGCCGTATAGTATAGGAATGGTGTCG |
| | CCTTGGGTGCCCGTAGGATAGCAAAAGTA | | CTTGGGTGCCCCAAGGCACTCGTCGATT |
| | TACTCATCGCT | | CGGAGCAGATC |
| 1749 | GCGAACGCCACTGCGGCCCCATCAGCAGC | 1805 | TTACTGCGGTGTACATTATTGCATGACT |
| | AATGAACAGTTATGTTATGATGTACACCA | | ACGAACAGTCAGTCGTACCACCGCCGAT |
| | CAGTTAATGGA | | ATCCACCACCA |
| 1750 | GCGAACGCCACTGCGGTCCCATCAGCAGC | 1806 | TTACTGCGGTGTACATTCTTGCATGACT |
| | AATGAACAGTTATGTTATGATGTACACCA | | ACGAACAGTCAGTCGTACCACCGCCGAT |
| | CAGTTAATGGA | | ATCCACCACCA |
| 1751 | GCTGCCGATCACCGAGATCGCGTTCGCGT | 1807 | CTCTCCTGAAGTGTCAGTTGAGCGCCTT |
| | CCGGCTTTCCGAGTGCGCGTGAACTACAG | | CGGTTTCGCCAGCGTGCGGCAGTTCAAC |
| | TTCTAGCATG | | GACACGATCC |
| 1752 | GGAAATTAATGAGCCGTTTGACCACTGAT | 1808 | CAGGGTTACTTTATACAACATTAATCTG |
| | CTTTTTGAAAATAAAGAGCAATGTTGTAC | | TATTTGAAATTTCGGAAGTGGCGCATCA |
| | ATCAAGATACA | | TGGTCCAGAAG |
| 1753 | GGAAATTAATGAGCCGTTTGACCACTGAT | 1809 | TAGTAATATTATATGCAACATTATTCTG |
| | CTTTTTGAAAATAAAGAGCAATGTTGTAC | | TATTTGAAATTTCGGAAGTGGCGCATCA |
| | ATCAAGATACA | | TGGTCCAGAAG |
| 1754 | GGTGAGGATGCGCTCGGAGTCGACCAGCG | 1810 | CGCTGAAAGCTAGTTTACTTTTCTATTC |
| | CCTTGGGGCACCCTAACGAAACCCATCCT | | GTTGGGGCATCCAAGACTGACGAAGCCG |
| | ATACTAGGGG | | ACTTTGGGAG |
| 1755 | GGTGAGGATGCGCTCGGAGTCGACCAGCG | 1811 | CGCTGAAAGCTAGTTTACTTTTCTATTC |
| | CCTTGGGGCACCCTAACGAAACCCATCCT | | GTTGGGGCATCCAAGACTGACGAAGCCG |
| | ATACTAGGGG | | ACTTTGGGAG |
| 1756 | GTCTTCTGGACCATGATGCGCTACTTCCG | 1812 | TGTATCTTGATGTACAACATTGCTCTTT |
| | AAATTTCAAATACAGAATAATGTTGCATA | | ATTTTCAAAAAGATCAGTGGTCAAACGG |
| | TAATATCACTA | | CTCATTAATTT |
| 1757 | GTGGATCACCTGGTTTTTCGTGTTCAGAT | 1813 | CTCCTTTTATTAGGGTTTGTGTCATCTA |
| | ACAGGCATGTAAAGTTTACATAAACCCTA | | CACACATACGAAGTGCTCCTGAGACAGA |
| | AAAAGATCGA | | AAGCGCATAT |
| 1758 | TAACACCAATTAAATGTTTAGTTCCCTCT | 1814 | GTCTTTATTTTTGGTATCCCGTTTCTTC |
| | TTGCGTCCAACGAGAGAAAACGAGGAACT | | TCCCTCCCTCATAGCTTGATCCGAAAAA |
| | AAACAATCTAA | | GTTACAGCTGG |
| 1759 | TAACACCAATTAAGTGTTTAGTTCCCTCT | 1815 | GTCTTTATTTTTGGTATCCCGTTTCTTC |
| | TTGCGTCCAACGAGAGAAAACGAGGAACT | | TCCCTCCCTCATAGCTTGAACCGAAAAA |
| | AAACAATCTAA | | GTTACAGCTGG |
| 1760 | TAACACCAATTAAGTGTTTAGTTCCCTCT | 1816 | ATGTTCTTTTTTGGTATCTCGTTTATTC |
| | TTGCGTCCAACGAGAGGAAACGAGGAACT | | TTCTTCCCTCATAGCTTGATCCGAAAAA |
| | AAACAATCTAA | | GTTACAGCTGG |
| 1761 | TAACACCAATTAAGTGTTTAGTTCCCTCT | 1817 | TGTTCTTTTTTTGGTATCTCGTTTCTTC |
| | TTGCGTCCAACGAGAGGAAATGAGGCACT | | TTCTTCCCTCATAGCTTGATCCGAAAAA |
| | AAACCAGTTGA | | GTTACAGCTGG |
| 1762 | TACAAAGTAGATGTCTTTTGTAGCCATTA | 1818 | CGTTCGTGCTTTGTCGTCACCTTGTTGG |
| | GGCGCATTAGGTTGACGCCAACAGGGTGA | | TGTAATTAGATTTACTCCATTAAGCCCC |
| | TGACAATATA | | AACGCATCAT |
| 1763 | TACCCGTTGCTTCGTTGTAGCAACACTAC | 1819 | TTTCTAAGCTTTTACAAGCAGAGCAACA |
| | GCACTCCACGTGTGGTGATAGGTCTTACC | | CACTCCACGTGATGCGTATTTGGAAATA |
| | CATATTATGGA | | AATCAGCCGGC |
| 1764 | TACCCGTTGCTTCGTTGTAGCAACACTAC | 1820 | TTTCTAAGCTTTTACAAGCAGAGCAACA |
| | GCACTCCACGTGTGGTGATAGGTCTTACC | | CACTCCACGTGATGCGTATTTGGAAATA |
| | CATATTATGGA | | AATCAGCCGGC |
| 1765 | TATCTTTTAACTGCAAGAGTACTACAGTT | 1821 | TCTACACGAGTAAGCAGACCTACACACT |
| | TCCACGTGCATTGACTGTCTACTTAGTAT | | CGATGTGAGCTGTTTGCGGGAACATATC |
| | CTTCCTACTAT | | GACGGGTTGCA |
| 1766 | TATCTTTTAACTGCAAGAGTACTACGGTT | 1822 | TCTTGGCGAGTGAGCAGACCTATACACT |
| | TCCACGTGCGTTGACTGTCTACTTAGTAT | | CGATGTGAGCTGTTTGCGGGAACATATC |
| | CTTCCTACTAT | | GACGGGTTGCA |
| 1767 | TATCTTTTAACTGCAAGAGTACTACGGTT | 1823 | TCCACACGTGTAAGCAGTCCTACACACT |
| | TCCACGTGCGTTGAGAGTACACTCTGTAT | | CGATGTGAGCTGTTTGCGGGAACATATC |
| | CTTCCTACTAT | | GACGGGTTGCA |
| 1768 | TATGCAACCCGTCGATATGTTCCCGCAAA | 1824 | ATAGTAGGAAGATACTAAGTAGACAGTC |
| | CAGCTCACATCGAGTGTATAGGTCTGCTC | | AACGCACGTGGAAACCGTAGTACTCTTG |
| | ACTCGCCAAGA | | CAGTTAAAAGA |
| 1769 | TATGCAACCCGTCGATATGTTCCCGCAAA | 1825 | ATAGTAGGAAGATACTAAGTAGACAGTC |
| | CAGCTCACATCGAGTGTATAGGTCTGCTC | | AACGCACGTGGAAACCGTAGTACTCTTG |
| | ACTCGCCAAGA | | CAGTTAAAAGA |
| 1770 | TCCCTTAGGTGCTAATAGCGCCACTAATT | 1826 | CCACACGTGTAAGCAGTCCTACACACTC |
| | CCACATGCGTTGAGAGTACACTCTGTATC | | GATGTGATGTGTTTGTGGGAATAAATCG |
| | TTCCTACTAT | | ACTGGTTGTA |
| 1771 | TCCCTTAGGTGCTAATAGCGCCACTAATT | 1827 | CCACACGTGTAAGCAGTCCTACACACTC |
| | CCACATGCGTTGAGAGTACACTCTGTATC | | GATGTGATGTGTTTGTGGGAATAAATCG |
| | TTCCTACTAT | | ACTGGTTGTA |
| 1772 | TCGGGGCACGGTATTGGTGATTCACGAGA | 1828 | TATTAGTTAGATGTCATAGACCGATTTA |
| | ACAAGGGACTGTAGGTTGATCTAGGACAC | | CAGCGGGCTCAACGACTGGGTTCGGTCC |
| | CTAACCAATA | | GTCGCGGGAC |
| 1773 | TTATTCTCTAATAAGTTTAACTACAGTCT | 1829 | GTGCTTTAGTCAACAATACTACGCTCTC |
| | CACAATGGCTCGGTTCTCCAATGACCAAC | | AACGTGTGGCGTATTTGGGAACATATCC |
| | CTATTCAACA | | ATACACTTAA |
| 1774 | TTATTCTCTAATAAGTTTAACTACAGTCT | 1830 | GTGCTTTAGTCAACAATACTACGCTCTC |
| | CACAATGGCTCGGTTCTCCAATGACCAAC | | AACGTGTGGCGTATTTGGGAACATATCC |
| | CTATTCAACA | | ATACACTTAA |
| 1775 | TTTAAATTTTGTCCTTTCTTCCCGCTATA | 1831 | TTTTTATTTTTATCCCCTAATTATACAT |
| | CCCACTTCCTCATATGTCAATAAGGATAA | | GGCATTGGCATTGTAAAAGATAAATAGT |
| | AAATATTATT | | TCGCCCACTC |
| 1944 | TAACACCAATTAAATGTTTAGTTCCCTCT | 1949 | GTCTTTATTTTTGGTATCCCGTTTCTTC |
| | TTGCGTCCAACGAGAGAAATCGAGGTACT | | TCCCTCCCTCATAGCTTGATCCGAAAAA |
| | AAACAAGCTAA | | GTTACAGCTGG |
| 1945 | ACAATCATCAGATAACTATGGCGGCACGT | 1950 | TTAATTTAGTATGGAAGTATGCACAATT |
| | GCATTAATGTATAATGTGTGTACTTCCAT | | GAGCAACCACGGTTGTATCCCGTCTAAA |
| | ATATTTATAC | | GTACTCGTAC |
| 1946 | AATGTTTGTAAAGGAGACTGATAATGGCA | 1951 | ATGGATAAAAAAATACAGCGTTTTTCAT |
| | TGTACAACTATACTAGTTGTAGTGCCTAA | | GTACAACTATACTCGTCGGTAAAAAGGC |
| | ATAATGCTTT | | ATCTTATGAT |
| 1947 | GTCTTCTGGACCATGATGCGCCACTTCCG | 1952 | TGTATCTTGATGTACAACATTGCTCTTT |
| | AAATTTCAAATACAGATTAATGTTGTATA | | ATTTTCAAAAAGATCAGTGGTCAAACGG |
| | AAGTAACCCTG | | CTCATTAATTT |
| 1948 | TTTAAATTTTGTCCTTTCTTCCCGCTATA | 1953 | TTTTTATTTTTATCCCCTAATTATACAT |
| | CCCGCTTCCTCATATGTCAATAAGGATAA | | GGCATTGGCATTGTAAAAGATAAATAGT |
| | AAATATTATT | | TCGCCCACTC |
| SEQ | | SEQ | |
| ID | | ID | |
| NO: | attB | NO: | attP |
| 1058 | TCTAACTCACGACACGTTGTACTCTTACC | 1389 | CAGTTTTTATTTTATGCCTTAATTATAC |
| | AACCGCACTTGCTCCCTCAAACGCTATAA | | ACCGCACTTGCGGTATGTCAATATGGCA |
| | TCCCCATAGTT | | AAAAGCTATTC |
| 1059 | CATTTTTACCTTGCTCTTCTCTCGAATTT | 1390 | AGTTTTATTTTTGTCTGTATAGGCTGTC |
| | CAGCATCTGCATGGCGCATAACATATTTA | | CGCATCTGCGGTATGCTTATAGGGACAA |
| | TGCGCTACAG | | AAATTATAAA |
| 1090 | ACAATCAACAAAGATGTATGGTGGTACAT | 1391 | TAACATATGTACGGAAGTATAGACACTC |
| | GCATTAATATCGGATGTATACCGACTAAA | | GATTAATATTTAATGTGTATACTTCCGT |
| | ACATTAATTC | | ATTTTTATTT |
| 1061 | TACAGACTTACATGGGACCATTCTATAGC | 1392 | TCAACTTTTAACCCTGTTTTAAGACCCA |
| | AGCTTTAAGATGCGTGAGGGACAAGATTA | | GTATTAAAATACTTAGCAATAAAACAGG |
| | CCAGACTCAG | | GGAATTGATA |
| 1062 | TGTAATTTCGGACACGAGTTCGACTCTCG | 1393 | TTGTATATTGCTAACAAAAGTTTAGCCT |
| | TCATCTCCACCAAAATATCAATATCCAAG | | CATCTCCACCATTTCTATCAATATACAT |
| | TCTTTGAATT | | AGGAAATAGT |
| 1063 | ATATGTTCCCGCAAACAGCACACGTTGAG | 1394 | TATCCCCTCCTCTCAAAACATGTAGAGA |
| | ACGGTAGTACTTTTGCAGTTAAAAGATAA | | CTGTAGTATTGATGTCAAGGGTTGATAA |
| | ATAAAGGACT | | GTAAGCGTGT |
| 1064 | TCGGCTTAGTGATGCCGAGTTCAGCTGGT | 1395 | TTTGCAATTGCTGGTGGTTCTGGTGCTT |
| | AAACCTTGGGTACTTGCTTCTCAGCTACT | | GGCCTTGGGCGATTGCGAGGTTTAAGGC |
| | TTCCCTCTTTT | | TTTCCACTTTT |
| 1065 | GTCTTCTGGACCATGATGCGCCACTTCTG | 1396 | TGTATCTTGATGTACAACATTGCTCTTT |
| | AAATTTCAAAAAGATCAGTGGTCAAACGG | | ATTTTCAAATACAGATTAATGTTGTATA |
| | CTCATTAATTT | | AAGTAGCCCTG |
| 1066 | CGGGCAAATTGCTGCCATATGGACCGGAG | 1397 | CTATTTATTAGATGTCTAAACAGTGCAT |
| | GCGGGACTTTAATTCCTTGGGCGCTTATT | | TACTACTCTACAACCTATATTAGACATC |
| | CCTGCCGCTGC | | TTATAAAAAGT |
| 1067 | TGATTTGATTGTATTGGATATTATGTTAC | 1398 | AATATAGTTGTATAAAAAGTCCTTTGCC |
| | CAGATGGCGAAGGTTATGATATTTGTAAA | | AGATGGCGAAGGACTTTTTGTACAACAA |
| | GAAATAAGAA | | AAAGTCACAA |
| 1068 | GCCCGTGGATTTGTTTCCAATGACGCATC | 1399 | CATAATATGGGTAAGACCTATCACCACA |
| | ACGTGGAGACGGTAGCACTTTTGTCCAAA | | TGTGGAGTGTGTTGCTCTGCTCGTAAAA |
| | CTTGATGTCGA | | GCCTAGAAACC |
| 1069 | GCTGGTGGTGGATATCGGCGGTGGTACGA | 1400 | TCCATTAACTGTGGTGCACATCATAACA |
| | CTGACTGTTCATTGCTGCTGATGGGGCCG | | TAACTGTTCGTAGTCATGCAAGAATGTA |
| | CAGTGGCGTTC | | CACCGCAGTAA |
| 1070 | GGAGGCTAAAACCTTTTTTGCCTGATAAT | 1401 | GGTGAAAATGTTGTAATAAGCGTCACAC |
| | CATACAAATAAGTGCCATTACAACAAATT | | ACTCAAATGTGTTATGCTTATACAAACA |
| | GCAGGTGTATC | | AAAATTAGAAG |
| 1071 | AGCTAAGTGTCCAAGCTGGCCCCCGATCC | 1402 | TACATAATTTCGTATATTAGATATTACC |
| | CAGTTTCAATAGTTTGGGGAATCTTTGTA | | AGTTTCAATTGGAAATACCTAATATACG |
| | AGTGGGAGAC | | AAAAAAGGCG |
| 1072 | ACAACAAAGACGCTAAGGTTTACGTGGTT | 1403 | AATTAAACTAAGATATTTAGATACGCTA |
| | AATGGAGACAGTCGTCAAGATATTACAGG | | CTCGAGACAAGAGTATCTAAATATCCTG |
| | TTCATTTACA | | TTTTTTTCGC |
| 1073 | CCCCAAAGTCGGCTTCGTCAGCCTTGGCT | 1404 | GAAGTATAGGGTTTATTTCATTGGGGTG |
| | GCCCGAAGGCCCTTGTTGATTCCGAGCGC | | CCCGAAGGCCCTCTGAAGTAAACTCTTA |
| | ATCCTCACCC | | TGACGCCCCG |
| 1074 | ATATCCCAAATGGAAAAGTTGTTAAACCG | 1405 | AAAAATTTAGTTGGTTATTGGTTACTGT |
| | TGTATAACGATACCAATCCCCCAACCTCC | | AACAAATCTTACGGTAACCAATAACCAA |
| | AAGTGGATAT | | CTTTAAAACT |
| 1075 | AACGTTTGTAAAGGAGACTGATAATGGCA | 1406 | ATGGATAAAAAAATACAGCGTTTTTCAT |
| | TGTACAACTATACTCGTCGGTAAAAAGGC | | GTACAACTATACTAGTTGTAGTGCCTAA |
| | ATCTTATGAT | | ATAATGCTTT |
| 1076 | GCCCAGGTGTGTCTGAGGTCATGGAAACG | 1407 | CGCAGGTTCGAATCCTGCAGGGCGCGCC |
| | GAAATCTTCCTCATTTATGCCCGTCTTAT | | ATTTCTTCAATTCCTGCACGACGACAAG |
| | CCGTTTCCGCT | | CTGATAGCCAT |
| 1077 | TAACACCAATTAAGTGTTTAGTTCCCTCT | 1408 | ATTTATAATTTTAGTTTCTCGTTTCTTC |
| | TTGCGTCCCTCATAGCTTGATCCGAAAAA | | TTCTTCCAACGAGAGAAAACGAGGAACT |
| | GTTACAGCTGG | | AAACAATCTAA |
| 1078 | CTGAGTGGGCGAACTATTTATCTTTTACA | 1409 | AATAATATTTTTATCCTTATTGACATAT |
| | ATGCCAAGCGGGTATAGCGGGAAGAAAGG | | GAGGAATGCCATGTATAATTAGGGGATA |
| | ACAAAATTTA | | AAAATAAAAA |
| 1079 | GAAACTATGGGGATTATAGCGTTTGAGGG | 1410 | GAATAACTTTTTGCCGTATTGACATACC |
| | AGCAAGTGCGGTTGGTAAGAGTAGCACGT | | GCAAGTGCGGTGTATAATTAAGGCATAA |
| | GTCGTGAATTA | | AATAAAAAACG |
| 1080 | CCGTCCCGCGACGGACCGAACCCAGTCGT | 1411 | TATTGGTTAGGTGTCCTAGATCAACCTA |
| | TGAGCCCCTTGTTCTCGTGAATCACCAAT | | CAGTCCGCTGTAAATCGGTCTATGACAT |
| | ACCGTGCCCC | | CTAACTAATA |
| 1081 | AGACTCAAAAACTGCAACCTTAAAGCTTT | 1412 | CTTCTTATTTAAACTAAGATATTTAGAT |
| | CACATTGCTTGAAAGCTTATTAACGCTAT | | ACATTGCTTGAGATAAGAGTATCTAAAA |
| | CAGTAACAAGT | | TTCACACTTTT |
| 1082 | GACGACGTCAAATGAGAAATCTGTTACAC | 1413 | TTTTTACAAAGAGGTATTTAGATACATG |
| | GTGTAACATTAGCAGTTAACCGCCGTTTT | | AGCTACAATGCCTGTATCTAAATACCTC |
| | AAATCGCAAAA | | TAAAGAAAGAC |
| 1083 | GTTAACAAGCACTTTAGACGGAATACAGC | 1414 | ACATAAATATATGGAAGTATACACACTA |
| | CATGGTTTATGCATGTACCGCCATAGCTT | | TACATTGGTTAATTGTGCATACTTCCAT |
| | TCTGTAAACT | | AAAATATTAA |
| 1084 | AGAACTGCGCTTTTTACAACAAGAGCATT | 1415 | TTTAGATTTTTCGTATTTACGATAACTT |
| | TTGTTTGTTTATATTTAAATACAAAAAAT | | TACATGTGTAAACATAACATAAATACTA |
| | CAAGTTATATA | | ATAAAATGTTA |
| 1085 | TATAGGCTGACATAAGTGTACTGTGGCGA | 1416 | TTTTCACTTCGTGTACATGGTGGAGTAT |
| | TTGTACTGATTCACTTCCCCATACCCAAA | | TAAACTGGTTTAACTCTCTACCATGTAC |
| | CATATTACAC | | ACTTTTTTTC |
| 1086 | TAAGGATAAGAAGGTTAAAGCATTTACAC | 1417 | TCTGAATATCAATAATTTTAGTAACCTT |
| | TTTTAGAGAGCCTTATTGTATTATCAGTA | | GATTGAAATCAAGGATAGTAAATTTCTT |
| | GTGGCATTTA | | TATATTTTCC |
| 1087 | ATTCCAACCATCACCAAGAACATCTTTAC | 1418 | AGATGCTCTCCCAGCTGAGCTAAACTCC |
| | TTCCAAGCTAAGCGACTTCCCTATCTCAC | | CTAGAGTTCGATACCATTTGAAAACACA |
| | AGGGGGCAAC | | GGAGAACGAG |
| 1088 | TCTGGCGGCAGTGCATTTCAAACACCATG | 1419 | TGTGCTCTTTTATTGTAGTTATATAGTG |
| | GTTTGGTCAATTGATGACTGGGCCACAGC | | TTTGGTCAATTAAACACAACCTAACTAC |
| | TTTTAGCTCA | | ATTAAATAAA |
| 1089 | TCCTAAGGGCTAATTGCAGGTTCGATTCC | 1420 | AATCCCCTGCCGCTTCAAGTAGATGTCT |
| | TGCAGGGGACACCAGATACCCTTCAAACG | | GCAGGGGACACCATTTATCAGTTCGCTC |
| | AAATCTACCTT | | CCATCCGTACC |
| 1090 | AAATAGAAAAATGAATCCGTTGAAGCCTG | 1421 | TAATGATTTTTAATGTTTCACGTTCAGC |
| | CTTTTTTATACTAACTTGAGCGAAACGGG | | TTTTTTATACTAAGTTGGCATTATAAAA |
| | AAGGTAAAAAG | | AAGCATTGCTT |
| 1091 | GACGAAATAGATATTTTTTGTGGCCATTA | 1422 | GATTTATGCTTTGTCGTCACCTTGTTGG |
| | AGCGCATTAGATTTACCCCATTTAATCCT | | TGTAATGAGGTTGTTACCAACAGGGTGA |
| | AAAGCATCAT | | TAACAAAGCT |
| 1092 | AACGAAGTAGATGTTTTTTGTTGCCATTA | 1423 | CGTTTATGCATTGTTGTCACCTTGTTGG |
| | GGCGCATTAGATTTACCCCATTTAATCCT | | TGTAATGAGGTTGACGACAACATGGTAG |
| | AATGCATCAT | | CGACAATATA |
| 1093 | AATATTAATAAGTTATATTGGGGGAACGT | 1424 | TTTTTTTACGTGAATGTTTTGTAACAAC |
| | GTGCGGTAGAAGTGGTACCATTCATGTCC | | TACAGTCTACCGCGTAACACACCATTCA |
| | TTACGAGATA | | TCAAAATTTA |
| 1094 | ATCGCTGTAGCGCATAAATACGTTATGAG | 1425 | GGTTTATAATTTTTGTCCCTATAAGCAT |
| | ACACGCAGATGCTGAAATTCGAGAAAAGA | | ACCGCAGATGCCGACAGACTATATAGAC |
| | GCAAAGTAAAG | | AAAAATAAAAC |
| 1095 | CATCTTTACTTTGCTCTTTTCTCGAATTT | 1426 | AGTTTTATTTTTGTCTATATTGGCTGTC |
| | CAGCATCTGCGTGTCTCATAACGTATTTA | | GGCATCTGCGGTATGCTTATAGGGACAA |
| | TGCGCTACAGC | | AAATTATAAAC |
| 1096 | ATCCCATGATGAGCCGAGATGACATAACC | 1427 | GTGGAAAATATAAAGAATTTTACTATCC |
| | CACCATTTCATTGAATGTCATTCTCTCAC | | TACATTTCAATTAAAGATACTAAATCTC |
| | CTTTATCAACC | | TTGATTTTTGA |
| 1097 | TCAAAAGTTAAGGGTTAAAGCATTTACGC | 1428 | CCTATTGAATGAGAGTTTTAGATACGCT |
| | TTTTAGAATGTTTGGTAGCATTGGTTACA | | TTTAGAATGTTTGGTATCTAAAACTCAC |
| | ATCACAGGAG | | GCTTTTTTGA |
| 1098 | GTTACTATAGCTCAGATGATTAAGGGACA | 1429 | AAACCATCAACAATTTTCCTCTGAGTGT |
| | CAGCCTAGGCTGTGTCCCTTAATTACGTA | | CATTTACTTCCCGTTTTTCCCGATTTGG |
| | AGCGTTGATA | | CTACATGACA |
| 1099 | GAATGATGCGTTGGGGCTTAATGGAGTAA | 1430 | TCTTTTGTCATCACCCTGTTGGCGTCAA |
| | ATCTAATGCGCCTAATGGCTACAAAAGAC | | CCTAATTACACCAACAAGGTGACGACAA |
| | ATCTACTTCG | | AGCATAAACG |
| 1100 | GGATCAAAAAGAACGACGATTCTTTAGTG | 1431 | TTTTCTTTTGTATCAAAATCAGTAGGAA |
| | TTTTTGATCCAACCATGGGTTCAGGTTCA | | CATAGAAATAATCTTACTGAGTTTAATA |
| | TTGATGTTAA | | CAATGCCGTG |
| 1101 | GGAAATTAATGAGCCGTTTGACCACTGAT | 1432 | CAGGGTTACTTTATACAACATTAATCTG |
| | CTTTTTGAAATTTCAGAAGTGGCGCATCA | | TATTTGAAAATAAAGAGCAATGTTGTAC |
| | TGGTCCAGAAG | | ATCAAGATGCA |
| 1102 | GTCTTCTGGACCATGATGCGCCACTTCCG | 1433 | TGTATCTTGATGTACAACATTGCTCTTT |
| | AAATTTCAAAAAGATCAGTGGTCAAACGG | | ATTTTCAAATACAGAATAATGTTGCATA |
| | CTCATTAATTT | | TAATATTACTA |
| 1103 | GTCTTCTGGACCATGATGCGCCACTTCCG | 1434 | TGTATCTTGATGTACAACATTGCTCTTT |
| | AAATTTCAAAAAGATCAGTGGTCAAACGG | | ATTTTCAAATACAGAATAATGTTGCATA |
| | CTCATTAATTT | | TAATATCACTA |
| 1104 | GTCTTCTGGACCATGATGCGCCACTTCCG | 1435 | TGTATCTTGATGTACAACATTGCTCTTT |
| | AAATTTCAAAAAGATCAGTGGTCAAACGG | | ATTTTCAAATACAGATTAATGTTGTATA |
| | CTCATTAATTT | | AAGTAACCCTG |
| 1105 | GTCTTCTGGACCATGATGCGCCACTTCCG | 1436 | TGTATCTTGATGTACAACATTACTCTTT |
| | AAATTTCAAAAAGATCAGTGGTCAAACGG | | ATTTTCAAATACAGAATAATGTTGCATA |
| | CTCATTAATTT | | TAATATTACTA |
| 1106 | ACAATCAACAAAGATGTATGGCGGTACAT | 1437 | TGATATAAGTACGGAAGTATAGACACTC |
| | GCATTAATATCGGATGTATACCGACTAAA | | GATTAATATTTAATGTGTATACTTCCGT |
| | ACATTAATTC | | ATTATTGTTT |
| 1107 | ATGAATTAATGTTTTAGTCGGTATACATC | 1438 | CTATAAAAATACGGAAGTATACACATTA |
| | CGATATTAATGCATGTACCGCCATACATC | | AATATTAATCAAGTGTCTATACTTCCGT |
| | TTTGTTGATT | | ACATAAGTTA |
| 1108 | ACAATCAACAAAGATGTATGGTGGTACAT | 1439 | TAACATATGTACGGAAGTATAGACACTT |
| | GCATTAATATCGGATGTATACCTACTAAA | | GATTAATATTTAATGTGTATACTTCCGT |
| | ACATTAATTC | | ATTTTTGTTT |
| 1109 | CTGTTTCAACAAATGATGCTCTTGGCCTT | 1440 | AAATACATATTCTCTTGTTGTCATCATG |
| | AATGGTGTAAACCTTATGCGTTTAATGGC | | TTGGTGTAAACCTAATTACACCAAGAGG |
| | GACAAAACATA | | ATGACGACAAA |
| 1110 | AGAAAAAGTGAATGTATTCACTGTTGGCT | 1441 | ATAATATAAAATACTGTTGTTCTATATG |
| | GGATTGGAGTTGCATGCACTCACCCTCCT | | GATTGGAGTTGCAACACAACTACAAATG |
| | ATGCTAAGTGT | | CAGTATAAAGG |
| 1111 | ATACGATTTCGGACAGGGGTTCGACTCCC | 1442 | AGCAGGGCGATCCTGAGTTTAATCTGGC |
| | CTCGCCTCCACCATTCAAATGAGCAAGTC | | TCGCCTCCACCAGCAAAGGTCACAATCG |
| | GTAAAAACATA | | TGTCGATGTCA |
| 1112 | AACCAGCTGTAACTTTTTCGGATCAAGCT | 1443 | TTAGATTGTTTAGTTCCTCGTTTCCTCT |
| | ATGAGGGACGCAAAGAGGGAACTAAACAC | | CGTTGGAAGAAGAATAAACGAGATACCA |
| | TTAATTGGTGT | | AAAAAGAACAT |
| 1113 | TATGCAACCCGTCGATATGTTCCCGCAAA | 1444 | ATAGTAGGAAGATACAGAGTGTACTCTC |
| | CAGCTCACGTGGAAACCGTAGTACTCTTG | | AACGCACATCGAGTGTGTAGGACTGCTT |
| | CAGTTAAAAGA | | ACACGTGTGGA |
| 1114 | TATCTTTTAACTGCAAGAGTACTACGGTT | 1445 | TCCACACGTGTAAGCAGTCCTACACACT |
| | TCCACGTGAGCTGTTTGCGGGAACATATC | | CGATGTGCGTTGAGAGTACACTCTGTAT |
| | GACGGGTTGCA | | CTTCCTACTAT |
| 1115 | AACCAGCTGTAACTTTTTCGGATCGAGTT | 1446 | TTAGATTATTTAGTACCTCGTTATCTCT |
| | ATGATGGACGTAAAGAGGGAACAAAGCAT | | CGCTGGAAGAAGAAGAAACGAGAAACTA |
| | CTAATAGGTGT | | AAATTATAAAT |
| 1116 | TTTTCCCCGAAAATCTTTAACACCGCTAT | 1447 | TATTTTGGTAGTTTATAGAAGTAATTTC |
| | CCGTTGATGTCCCAGCTCCTCCAAAGAAA | | AGTTGATGTTCACTCCATTAATTACCAA |
| | ACTAAATATT | | AATTTAAAAA |
| 1117 | GGATCAGAAGGTTAGGGGTTCGACTCCTC | 1448 | AAATTTGTTAGGGTAAAAAAGTCATAGT |
| | TTGGGTGCGCCATTTAAAAATAATAATAA | | TGGGTGCGCCATCGATTAACCCTAACTG |
| | GACTGTAGCCT | | ATAAATAAAAA |
| 1118 | TTTTCCCCCGAAAATCTTTAACACCACTA | 1449 | TTATTTTGGTAGTTTATAGAAGTAATTT |
| | TCTGTTGATGTCCCAGCTCCTCCAAAGAA | | CAGTTGATATTCACTCCATTAATTACCA |
| | AACTAAATAT | | AAAAAACAGG |
| 1119 | GTAAACTAAAATATGCCCAGACCCCATTG | 1450 | TATGGAATTGTATCAATCTCGGCGTGGT |
| | CGTTATCGATAATTTTTAGTTCTTCTGGT | | TTTGTCCGTTGCCACTCTGAAATTGATA |
| | TTTAAATTAC | | CAATGTAACA |
| 1120 | GTAAACTAAAATATGCCCAGACCCCATTG | 1451 | TATGGAATTGTATCAATCTCGGCGTGGT |
| | CGTTATCGATAATTTTTAGTTCTTCTGGT | | TTTGTCCGTTGCCACTCTGAAATTGATA |
| | TTTAAATTAC | | CAATGTAACA |
| 1121 | CTTGTGGATCACCTGGTTTTTCGTGTTCA | 1452 | TGTCTCTTTTTATTAGGGTTTATATCAA |
| | GATACACACATACGAAGTGCTCCTGAGAG | | CTACACACATGTAAAGTAGACATAAACA |
| | AGAAAGCGCAT | | GCAAAAATTTG |
| 1122 | GAAGGCAGACCATTAACAGGAAGGGATGG | 1453 | TAAAGATCGTAAAAAAGAAATAGAGTTC |
| | AGCATTTGACCTTACCCAGAAAAAGTGGA | | CGAATTACACCATTTATAAAAAAGCTGC |
| | GAGAAAGAAA | | TGGAGGCAAG |
| 1123 | GGAAATTAATGAGCCGTTTGACCACTGAT | 1454 | TAGTAATATTATATGCAACATTATTCTG |
| | CTTTTTGAAATTTCGGAAGTGGCGCATCA | | TATTTGAAAATAAAGAGCAATGTTGTAC |
| | TGGTCCAGAAG | | ATCAAGATACA |
| 1124 | GTCTTCTGGACCATGATGCGCCACTTCCG | 1455 | TGTGTCTTGATGTACAACATTACTCTTT |
| | AAATTTCAAAAAGATCAGTGGTCAAACGG | | ATTTTCAAATACAGAATAATGTTGCATA |
| | CTCATTAATTT | | TAATATTACTA |
| 1125 | GCTTCTGCTTGGATTTTACGCCATCCAGC | 1456 | TTCATTATTTTAATAGAGATAGAAATCA |
| | CAATATGCAAGTGATCGCCGGTACGATGA | | ACCATGCACATGGTAGCATGAGTGTTCT |
| | ACGTAGGGCGA | | ATGAAAAAAGA |
| 1126 | GTCTTCTGGACCATGATGCGCCACTTCCG | 1457 | TGTATCTTGATGTACAACATTACTCTTT |
| | AAATTTCAAAAAGATCAGTGGTCAAACGG | | ATTTTCAAATACAGAATAATGTTGCATA |
| | CTCATTAATTT | | TAATATTACTA |
| 1127 | AGCTTTTATTGCAAGAAAAATGGGTTATA | 1458 | TATTTATATAAAATAGTGTTTTTGTAAA |
| | AGTACACATCAGGTTATAGTAATATCGAA | | GTACACATCACCATATTTGACAAAAAAC |
| | AAAGGAAGCG | | CTATAAATAA |
| 1128 | AACCAGCTGTAACTTTTTCGGATCGAGTT | 1459 | TTAGATTGTTTAGTATCTCGTTATCTCT |
| | ATGATGGACGTAAAGAGGGAACAAAGCAT | | CGTTGGAGGGAGAAGAAACGGGATACCA |
| | CTAATAGGTGT | | AAAATAAAGAC |
| 1129 | ACGTTTGTAAAGGAGACTGATAATGGCAT | 1460 | TGGATAAAAAAATACAGCGTTTTTCATG |
| | GTACAACTATACTCGTCGGTAAAAAGGCA | | TACAACTATACTCGTTGTAGTGCCTAAA |
| | TCTTATGATGG | | TAATGCTTTTA |
| 1130 | ACAATCATCAGATAACTATGGCGGCACGT | 1461 | TTAATAAACTATGGAAGTATGTACAGTC |
| | GCATTAACCACGGTTGTATCCCGTCTAAA | | TTGCAATGTTGAGTGAACAAACTTCCAT |
| | GTACTCGTAC | | AATAAAATAA |
| 1131 | AACAATCTGCAAACATGTATGGCGGTACA | 1462 | TTAATTTTTGTACGGAAGTAGATACTAT |
| | TGTATCAACATTGGTTGTATTCCTACAAA | | CTTTCAATATCCATGTTACTTAGTGCCA |
| | GACACTCATT | | TACAAAAACC |
| 1132 | ACAGCCTGTGGATATGTTTGCACAGACTG | 1463 | GTCTTTTTACCTTATATAACAGTTTCAT |
| | CTCACGTGGAGTGTGTAGTTAAGCTAATC | | GCACGTGGAGACGGTAGTATTGATGTCA |
| | AAGGTAAATCA | | CGAAAAGAAAA |
| 1133 | CGAGACGAGAAACGTTCCGTCCGTCTGGG | 1464 | TGTTATAAACCTGTGTGAGAGTTAAGTT |
| | TCAGTTGGGCAAAGTTGATGACCGGGTCG | | TACATGCCTAACCTTAACTTTTACGCAG |
| | TCCGTTCCTT | | GTTCAGCTTA |
| 1134 | ATTCTCCTTTAACGAATGAAGCGACTAAT | 1465 | TTGACTTTTGACATCAATACTACGCACT |
| | TCGATATGATGGGTTTGCGGGAAAAGATC | | CCACATGGCTTGAGAGGACAGAATGAAT |
| | TACAGGCTGAA | | GTCATTTGAGT |
| 1135 | CAGCCGGCTGATTTATTTCCAAATACGCA | 1466 | TCCATAATATGGGTAAGACCTATCACCA |
| | TCACGTGGAGTGCGTAGTGTTGCTACAAC | | CACGTGGAGTGTGTTGCTCTGCTTGTAA |
| | GAAGCAACGGG | | AAGCTTAGAAA |
| 1136 | TATGCAACCCGTCGATATGTTCCCGCAAA | 1467 | ATAGTAGGAAGATACAGAGTGTACTCTC |
| | CAGCTCACGTGGAAACCGTAGTACTCTTG | | AACGCACATCGAGTGTGTAGGACTGCTT |
| | CAGTTAAAAGA | | ACACGTGTGGA |
| 1137 | AACAGAAGAAGGGAAGTTCTACCTATTGA | 1468 | CCGAAGCATCGTATCAATGCTTCGGTCA |
| | TACCTTTGGTGGAGCTGAGGAGACGATAT | | ATGTTTGGCAAAGGGCACGAGTTTGATA |
| | CTAGAACCGAT | | CAAAATGCACC |
| 1138 | AACAGAAGAAGGGAAGTTCTACCTATTGA | 1469 | CCGAAGCATCGTATCAATGCTTCGGTCA |
| | TACCTTTGGTGGAGCTGAGGAGACGATAT | | ATGTTTGGCAAAGGGCACGAGTTTGATA |
| | CTAGAACCGAT | | CAAAATGCACC |
| 1139 | AACAGAAGAAGGGAAGTTCTACCTATTGA | 1470 | CCGAAGCATCGTATCAATGCTTCGGTCA |
| | TACCTTTGGTGGAGCTGAGGAGACGATAT | | ATGTTTGGCAAAGGGCACGAGTTTGATA |
| | CTAGAACCGAT | | CAAAATGCACC |
| 1140 | GTCTCGCTCGCCCACCGCGGGGTGCTCTT | 1471 | GTAGCCACTTGTTTTACACGTCTTGTCT |
| | TCTGGACGAGGCCCCGGAGTTCTCGGGGA | | CTGGACGAGGCATGTAAAACAGGTGGGC |
| | AGGCGCTGGAC | | TTGATCAGCTA |
| 1141 | CACTACAGTATGCAGATTTTGCAGCTTGG | 1472 | TATGATAATTTTAGTATTCATGATTGGT |
| | CAGCGTGAATGGCTACAAGGTGAGGCGTT | | TGTTTGAATAGCCCGTTATGAATACTAA |
| | AGAGCAACAGC | | AAATTCCACTC |
| 1142 | TCATCACTACTTAATATATCCATAAGAGA | 1473 | ACCCTTAAACATATAACATGTTTAAGGG |
| | AATTTCATTTCCTTCTTTGTCTACTCCTA | | TATTCATTACCCACTTCATGTTGTATGT |
| | TAGGATCTTG | | TATGTAAAAA |
| 1143 | TCTGGTGGCAGTGCATTTCAAACACCGTG | 1474 | TGTGCTCTTTTGTTGTATTTATATGGCG |
| | GTTTGGTCAATTGATGACTGGGCCACAGC | | TTTGGTCAATTAAACACAACCTAACTAC |
| | TTTTAGCTCA | | ATCAAATGAA |
| 1144 | GTTTTTTGTAGCCATTAGGCGCATGAGGT | 1475 | GTCGTCACCTTGTTGGTGTAATTAGATT |
| | TTACGCCATTAAGCCCTAAAGCGTCATTC | | AACCCCAACAGGGTGATAACAAAAGAAG |
| | GTCGAAACAGC | | GATTTTTTAAT |
| 1145 | GATCACCCAGGACGTCTGCGCCTTCTACG | 1476 | CCTGTATTGTGCTACTTAGAGCATAAGG |
| | AGGACCATGCCCTCTACGACGCCTACACG | | CGACCATGCCTTACAAGCTCAAAATAGC |
| | GGCGTGGTGGT | | ACACGTTTCCG |
| 1146 | GCAACCGGCATCAGTGTAATACCGATAAT | 1477 | CAAATAATGTAGTACCCAAATTAAGTTT |
| | CGTAACAACAGAGCCTGTCACGACCGGCG | | CACACAAGCAACCTTAATCGGGTACTAC |
| | GAAAAAACGA | | TTAATATCTA |
| 1147 | GTGAGGATGCGCTCGGAGTCGACCAGCGC | 1478 | TCTGAGAATTAGTATATTTTCCTATTCG |
| | CTTGGGGCATCCAAGACTGACGAAGCCGA | | CAGGGGCACCCTAACGAAACCCATCCTA |
| | CTTTGGGAGT | | TACTAGGGGC |
| 1148 | ACAAGACCCCATCGGAACAGATAAAGAAG | 1479 | ATACCAATAACATATAAAGAGTAGTGTG |
| | GTAATGAAATAAGTCTTTTAGATATACTT | | TAATGAAATAAACACTACTATTTATATG |
| | GGCACAGAGG | | TTATTTTCTA |
| 1149 | GCTGGTGGTGGATATCGGCGGTGGTACGA | 1480 | TCCATTAACTGTGGTGTACATCATAACA |
| | CTGACTGTTCATTGCTGCTGATGGGGCCG | | TAACTGTTCGTAGTCATGCAAGAATGTA |
| | CAGTGGCGTTC | | CACCGCAGTAA |
| 1150 | CCATCATAAGATGCCTTTTTACCGACGAG | 1481 | AAAGCATTATTTAGGCACTACAACTAGT |
| | TATAGTTGTACATGCCATTATCAGTCTCC | | ATAGTTGTACATGAAAAACGCTGTATTT |
| | TTTACAAACG | | TTTTATCCAT |
| 1151 | CCACTCCCAAAGTCGGCTTCGTCAGTCTT | 1482 | GCCCCTAGTATAGGATGGGTTTCGTTAG |
| | GGATGCCCCAAGGCGCTGGTCGACTCCGA | | GGTGCCCCTACGAATAGAAAAATATACT |
| | GCGCATCCTC | | AATTCTCAGG |
| 1152 | CACTCCCAAAGTCGGCTTCGTCAGTCTTG | 1483 | CCCCCAGTGTAGGATTTATATCACTAGG |
| | GATGCCCCAAGGCGCTGGTCGACTCCGAG | | TTGCCCCAACGAATAGAAAAGTAAACTA |
| | CGCATCCTCA | | GCTTTCAGCG |
| 1153 | ACCAGCTGTAACTTTTTCGGATCAAGCTA | 1484 | TAGATTGTTTAGTATCTCATTATCTCTC |
| | TGAGGGACGCAAAGAGGGAACTAAACACT | | GTTGGACGGAGACGAATCGAGAAACTAA |
| | TAATTGGTGTT | | AATTATAAATA |
| 1154 | AGTTCAGCCCGTGGATTTGTTTCCAATGA | 1485 | TCGTTCCATAATATGGGTAAGACCTATC |
| | CGCATCATGTGGAGTGCATAGCGTTGATA | | ACCACACATCGAGTGTGTGGTTCTGCTC |
| | CAAAGAGTGA | | GTAAAAGCCT |
| 1155 | AGAAATCACTCAGCAAGAGTTAGCCAGGC | 1486 | CCCCCTCGTGTTATTGTGGGTACATGAT |
| | GAATTGGCAAACCTAAACAGGAGATTACT | | ATTTGGCAACCCGAATGTAGTCAACCCA |
| | CGCCTATTTAA | | AAATAACTAAA |
| 1156 | CAGCCGACTGATTTGTTTCCGAATACGCA | 1487 | ATATGACATCAATGCCATCAACTCGAGC |
| | TCACGTGGAGTGCGTAGTGTTGCTACAAC | | CACGTGGAGTGTGTGGTTCTGCTCGTAA |
| | GAAGCAACGGG | | AAGCCTAGAAA |
| 1157 | GTCTTCTGGACCATGATGCGCCACTTCTG | 1488 | TGTATCTTGATGTACAACATTGCTCTTT |
| | AAATTTCAAAAAGATCAGTGGTCAAACGG | | ATTTTCAAATACAGATTAATGTTGTATA |
| | CTCATTAATTT | | AAGTAGCCCTG |
| 1158 | TGATTTGATTGTATTGGATATTATGTTAC | 1489 | AATATAGTTGTATAAAAAGTCCTTTGCC |
| | CAGATGGCGAAGGTTATGATATTTGTAAA | | AGATGGCGAAGGACTTTTTGTACAACAA |
| | GAAATAAGAA | | AAAGTCACAA |
| 1159 | AAAATGTGTAGACATGTTTCCTTATACGA | 1490 | CGAAAGACATCAATACTGTCCTCTCGAG |
| | CACATGTTGAGACGGTAGTGTTAATGGAG | | CCATGTTGAGTGCGTCACATTGATGTCA |
| | AGAAAGTAAGA | | AGGGTTTAGAA |
| 1160 | AATAACAAACTATTTTTTATAGAAACATG | 1491 | AAAGAAAAAATTCTTTATTTCTACATAC |
| | GGGATGTCAGATGAATGAAGAGGATTCCG | | GGTTGTCCGTATGTAGAAAATAGTAGGA |
| | AAAAATTATC | | ATATATGAGA |
| 1161 | TAACACCAATTAAGTGTTTAGTTCCCTCT | 1492 | CTTTATTTTTTTTGTATCCCATTTCCTC |
| | TTGCGTCCCTCATAGCTTGATCCGAAAAA | | TCCCTCCAACGAGAGGAAATGAGGCACT |
| | GTTACAGCTGG | | AAACCAGTTGA |
| 1162 | TAACACCAATTAAGTGTTTAGTTCCCTCT | 1493 | TGTTCTTTTTTTGGTATCTCGTTTCTTC |
| | TTGCGTCCCTCATAGCTTGATCCGAAAAA | | TTCTTCCAACGAGAGAAAACGAGGTACT |
| | GTTACAGCTGG | | AAATAAGCTAA |
| 1163 | TAACACCAATTAAATGTTTAGTTCCCTCT | 1494 | TGTTCTTTTTTTGGTATCTCGTTTCTTC |
| | TTGCGTCCCTCATAGCTTGATCCGAAAAA | | TTCTTCCAACGAGAGAAAACGAGGTACT |
| | GTTACAGCTGG | | AAATAAGCTAA |
| 1164 | GGTGAGGATGCGCTCGGAGTCGACCAGCG | 1495 | CTTAAAGATTGAGTTTACTTTTGCAGTC |
| | CCTTGGGGCATCCAAGACTGACGAAGCCG | | ATTGGGGCACCCTAACGAAACCCATCCT |
| | ACTTTGGGAG | | ATACTAGGGG |
| 1165 | TTTATCCCGTAAGGACATGAATGGTACCA | 1496 | TAAATTTTGATGAATGGTGTGTTACGCG |
| | CTTCTACCGCACACGTTCCCCCAATATAA | | GTAGACTGTAGTTGTTACAAAACATTCA |
| | CTTATTAATA | | CGTAAAAAAA |
| 1166 | TATCCCGTAAGGACATGAATGGTACCACT | 1497 | AATATTAATGAGTGTTATGTAACTAGAA |
| | TCTACCGCACACGTTCCCCCAATATAACT | | AGACCGCAATAGTTACAAAACATTCATT |
| | TATTAATATT | | AAAAATAACC |
| 1167 | GGATCAAAAAGAACGACGATTCTTTAGTG | 1498 | TTTTCTTTTGTATCAAAATCAGTAGGAA |
| | TTTTTGATCCAACCATGGGTTCAGGTTCA | | CATAGAAATAATCTTACTGAGTTTAATA |
| | TTGATGTTAA | | CAATGCCGTG |
| 1168 | CACTCCCAAAGTCGGCTTCGTCAGTCTTG | 1499 | CCCCTAGTATAGGATGGGTTTCGTTAGG |
| | GATGCCCCAAGGCGCTGGTCGACTCCGAG | | GTGCCCCAATGATTGCAAAAGTAAACTC |
| | CGCATCCTCA | | AATCTTTAAG |
| 1169 | GTGGATCACCTGGTTTTTCGTGTTCAGAT | 1500 | CTCTTTTTATTAGGGTTTATATCAACTA |
| | ACAGGCATACGAAGTGCTCCTGAGACAGA | | TACACATGTAAAGTAGACATAAACAGCA |
| | AAGCGCATATC | | AAAATTTGATA |
| 1170 | TCTATTTAAATTGTCTATTTTATTGACAG | 1501 | AAGATATTACCCTGAATGAAGTCTTACG |
| | GGGACCAAATTGAAGTGGCCGCTAATCAG | | TCGTCAATCTCTGCTAAGATTACCAAAT |
| | TTCCTTCAAAA | | AACCCCGACAA |
| 1171 | TCTATTTAAATTGTCTATTTTATTGACAG | 1502 | AAGATATTACCCTGAATGAAGTCTTACG |
| | GGGACCAAATTGAAGTGGCCGCTAATCAG | | TCGTCAATCTCTGCTAAGATTACCAAAT |
| | TTCCTTCAAAA | | AACCCCGACAA |
| 1172 | CCGAGCTGCCGATCACCGAGATCGCGTTC | 1503 | TGGCCTCTCCTGAAGTGTCAGTTGAGCG |
| | GCGTCCGGTTTCGCCAGCGTGCGGCAGTT | | CCTTCGGCTTTCCGAGTGCGCGTGAACT |
| | CAACGACACGA | | ACAGTTCTAGC |
| 1173 | GATCACCCAGGACGTCTGCGCCTTCTACG | 1504 | CCTGTATTGTGCTACTTAGAGCATAAGG |
| | AGGACCATGCCCTCTACGACGCCTACACG | | CGACCATGCCTTACAAGCTCAAAATAGC |
| | GGCGTGGTGGT | | ACACGTTTCCG |
| 1174 | ACCAGCTGTAACTTTTTCGGATCAAGCTA | 1505 | TACGTTGTTTAGTACCTCAATTTCTCTC |
| | TGAGGGACGCAAAGAGGGAACTAAACACT | | TCTGGACGGAGACGAATCGAGAAACTAA |
| | TAATTGGTGTT | | AATTATAAATA |
| 1175 | ACTGGCGAAGCGATTCTTGGTGCGAACAT | 1506 | AAACCCATTTTTACCTTATGTAAAAAAA |
| | TTTCCGTGATTTTTTTGCGGGCATCCGTG | | TCACGTGATATGTTTACCAAATGACAAA |
| | ATGTGGTCGGC | | AATGATATAAT |
| 1176 | TTCTAACTCACGACACGTTGTGCTCTTAC | 1507 | GGTTTTTTATTTGTATGCCATAATTATA |
| | CAACCGCACTCGCTCCCTCAAACGCTATA | | CACCGCACTTGCGGTATGTCAATAAGAC |
| | ATCCCCATAG | | ATACGAATTT |
| 1177 | GGTGAGGATGCGCTCGGAGTCGACCAGCG | 1508 | CTTAAAGATTGAGTTTACTTTTGCAGTC |
| | CCTTGGGGCATCCAAGACTGACGAAGCCG | | ATTGGGGCACCCTAACGAAACCCATCCT |
| | ACTTTGGGAG | | ATACTAGGGA |
| 1178 | GCTGTGGCGGTTCCAAATTGGTGAGGCGC | 1509 | AACGTGCCTTTGTCGCAGCTGCCAAAGT |
| | CAAATCCGACGTCCCCCCATCCTGAGTAG | | TTAGCCGCTCAACTTGGTGGCGACCGAT |
| | CAGTCGGGTTT | | GCCTGCGGTCA |
| 1179 | AAAATCTAAATTTTCTTTTGGCAGACCTT | 1510 | CCTTTAATTTTTGGGTTAAAGGAACATT |
| | CTTCGCTACTCGTAATATTACCTAACACG | | GACTCTAGTGAGTGTTATATTAACCCAA |
| | GAACGAAATAA | | AAAGAGCCTAC |
| 1180 | TACAGACTTACATGGGACCATTCTATAGC | 1511 | TCAACTTTTAACCCTGTTTTAAGACCCA |
| | AGCTTTAAGATGCGTGAGGGACAAGATTA | | GTATTAAAATACTTAGCAATAAAACAGG |
| | CCAGACTCAG | | GGAATTGATA |
| 1181 | ATCACGATGGGGAGCAGTTCGATGTACCC | 1512 | TCCGTGATAGGCCGCGTGGCGTCGCCTC |
| | CATCTCCAGGTCCTTCACCACATAGTCCG | | AGCACCACCACTTACCCAAAACCCAACC |
| | CCGCCCCCTGC | | CTTATCGGTTG |
| 1182 | GGTTAAGTGTATGGATATGTTCCCAAATA | 1513 | ACTCAAATGACATTCATTCTGTCCTCTC |
| | CTCCACATTGTGAGACGTGCGTACTTTTG | | AAGCCACGTTGAGTGCGTAGTATTGATG |
| | TCCCACAAAA | | TCAAGGGTTG |
| 1183 | AACCAGCTGTAACTTTTTCGGATCAAGCT | 1514 | TCAACTGGTTTAGTGCCTCATTTCCTCT |
| | ATGAGGGACGCAAAGAGGGAACTAAACAC | | CGTTGGAAGAAGAAGAAACGAGATACCA |
| | TTAATTGGTGT | | AAAAAAGAACA |
| 1184 | CGTTTATGAATGACTTGATTTTTGGTATG | 1515 | AGACATTCATTTTTATTAGGGTTTATGT |
| | TAAAGTATAAGCAGACAAAATGCTCCTGG | | AAAGTATAAGCATGTAAACTTAACATAA |
| | GATAAAAAGC | | ATACAAATAA |
| 1185 | TCTTCAAGATCCAATAGGAATAGATAAAG | 1516 | AACATTTTACAAGTATATAACATGTAAT |
| | AAGGCAATGAAATCTCTTTAATGGATGTT | | AGGCAATGAATTACCCTGGACAAGTTGT |
| | TTAGGTACAG | | CAGTCTAGGG |
| 1186 | AACAGTTCCTTTTTCAATGTTACTGTAAC | 1517 | TTATTTATAGGTTTTTTGTCAAATACGG |
| | CTGATGTGTACCTATAGCCCATCCGTCGC | | TGATGTGTACTTTACAAAAACACTATTT |
| | GCAATGAAAG | | TATATAAATA |
| 1187 | GGGGCAAATTGCTGCGATTTGGGTTGGAG | 1518 | AGAATAATTATATGTCTTCTATTGGCGG |
| | GGGGAACGTTGATTCCATGGGCGCTCATT | | TAATACCCCAGCATAGACAATATACATA |
| | CCAGCTGCTG | | TAATCTTTCT |
| 1188 | GTCTTCTGGACCATGATGCGCCACTTCCG | 1519 | TGTATCTTGATGTACAACATTGCTCTTT |
| | AAATTTCAAAAAGATCAGTGGTCAAACGG | | ATTTTCAAATACAGAATAATGTTGCATA |
| | CTCATTAATTT | | TAATATTACTA |
| 1189 | ATGAATTAATGTTTTAGTCGGTATACATC | 1520 | GGTTATTTTTACGGAAGTATACACATTA |
| | CGATATTAATGCATGTACCGCCATACATC | | AATATTAATCAGGTGTCTATACTTCCGT |
| | TTTGTTGATT | | ACATATGTTA |
| 1190 | GATGTTCGTAGCAACTATGGGAGGAACCG | 1521 | GGTTTTTATATGTGCGTTATGTAACAAG |
| | GTGCAACATTAGTTGTTCCATTTATGTTT | | CACCACGGCTATAGTTACATAACCCACA |
| | ATGTGGTTAA | | TTAAAATATA |
| 1191 | ATGAATTAATGTTTTAGTCGGTATACATC | 1522 | TTATTTTTTTACGGAAGTATACACAATA |
| | CGATATTAATGCATGTACCGCCATACATC | | AATATTAATAGAGTGTCTATACTTCCGT |
| | TTTGTTGATT | | ACATATGTTA |
| 1192 | ACAGTTTACAGAAAGCTATGGCGGTACAT | 1523 | TTGATATTTTATGGAAGTATGCACAATT |
| | GCATAAACCATGGCTGTATTCCGTCTAAA | | AACCAATGTATAGTGTGTGTACTTCCAT |
| | GTGCTTGTTA | | ATATTTATGC |
| 1193 | ATAGAAGCACACTGATGATGAGCAAGACC | 1524 | AATTGGAAAATATAAATAATTTTAGTAA |
| | ACCAACATTTCCACAAGTGTGAAAGCTTT | | CCTACATCTCAATAAAGGATAGTAAAAT |
| | AACCTTAGCT | | TATTGATTTT |
| 1194 | ACCAGCTGTAACTTTTTCGGATCAAGCTA | 1525 | TACGTTGTTTAGTACCTCAATTTCTCTC |
| | TGAGGGACGCAAAGAGGGAACTAAACACT | | TCTGGACGGAGACGAATCGAGAAACTAA |
| | TAATTGGTGTT | | AATTATAAATA |
| 1195 | GGATTTCGTTGCACTGATGGGCGGTACTG | 1526 | CTCTTTTTTATGTATGGTTTGTAACAAT |
| | GCGCGACTTTACTCGTTCCTTATTTATTT | | ATCCACCTACAAAGTGCTAAACCATACA |
| | ATATTTCTTT | | TGTTAAAAAT |
| 1196 | GGATTTCATTGCACTGATGGGCGGTACTG | 1527 | TCTTTTTTTATGTATGGTTTGTAACAAT |
| | GCGCGACTTTACTCGTTCCTTATTTATTT | | ATCCACCTACAAAGTGCTAAACCATACA |
| | ATATTTCTTT | | TGTTAAAAAT |
| 1197 | TATATGTCTTCATATAATCGAGCAATGTG | 1528 | TTAGGGTTACCATTGATCATGAAGACCA |
| | TTCAGATAGTTGAGTCCGTATAATTGTGT | | TTATATCATCCAGCTCATAGTATTTTGT |
| | AAAAAGCTAG | | CTCTTTCTTT |
| 1198 | GCGCGCCGACTTTATGCAGGATCACATTG | 1529 | TTCAAGTCTAGGATACGAACAGTACGTT |
| | CTGGGCACTTCGAACAGAAAGTAGCCGAG | | TGCGCACACGATAACGTGCCGTTCGTAA |
| | GAAGAAGATG | | ACCGACGAGC |
| 1199 | TTCGTTAATTGGAGCTACGGCCATTGGTG | 1530 | AGATGTGATGTTAATTATTCTGGTCAGT |
| | GACCTCCTGACCACCCCCACTCGTAAGTC | | ACCTCCTGACCGGATTAATTAATATCAC |
| | ATAATAATTAC | | TAGGAAATGGC |
| 1200 | TAATGCATACATTGTCGTTGTCTTCCCAG | 1531 | TTAATATCAGTTGTATTTATACTACTAG |
| | AACCAGTCGGTCCAGTAAACACGAGTAGC | | CTCTGTAGCTAACGTTATATAAATACAC |
| | CCCTGTGAAT | | TTAAAATAAA |
| 1201 | GCTCTGCAAAAGCTTGATCGTCGGTTCAA | 1532 | AAACCCTTGATATACCAATAGTTTCAAA |
| | ATCCGTCTACCGCCTTTTAATATTCTAAA | | TCCGTCTACCGCCTTTATTATAGGATTT |
| | AAACCTAGGA | | TGTCCGAATT |
| 1202 | ACAATCATCAGATAACTATGGCGGCACGT | 1533 | TTAATTTAGTATGGAAGTATGCACAATT |
| | GCATTAACCACGGTTGTATCCCGTCTAAA | | GAGCAATGTATAATGTGTGTACTTCCAT |
| | GTACTCGTAC | | ATATTTATAC |
| 1203 | ATGTACGAGTACTTTAGACGGGATACAAC | 1534 | GTATAAATATATGGAAGTACACACATTA |
| | CGTGGTTAATGCACGTGCCGCCATAGTTA | | TACATTGCTCAATTGTGTATACTTCCAT |
| | TCTGATGATT | | ACTAAATTAA |
| 1204 | ATGAAGATTATAATAATTGGAGGTGGCTG | 1535 | TCACGTGTTTTAATGGAGTTTTAACTGG |
| | GTCTGGATGTGCAGCAGCCATAACAGCTA | | TCTGGATGTGCAGCACAGGTAAAACTAC |
| | AAAAGGCAGGT | | ACTAATTATTA |
| 1205 | AACCCCAAAGTCGGCTTCGTCAGCCTTGG | 1536 | TAGAAGTATAGGGTTTGTTTCATTGGGG |
| | CTGCCCGAAGGCCCTCGTCGATTCCGAGC | | TGCCCGAAGGATGGTTGAGATATACTTT |
| | GCATCCTCAC | | TGGCGAGCAG |
| 1206 | GAATCTAAATTTTCTTTCGGTAATCCTTC | 1537 | CTTTAATTTTTGGGTTAAAGGAACATTG |
| | TTCACTACTCGTAATATTTCCTAATACAG | | ACTCTACTAAGTGTTATATTAACCCAAA |
| | AACGAAATAAA | | AAAGAGCCTTC |
| 1207 | CTGGCTTGATTAATAGTTTAAAAGTCTTG | 1538 | TCCTGAATGGTTACTACGATTGGTTTGG |
| | GCTGGTGTCACGAACGGTGCAATAGTGAT | | TTGGTGTTATTGCTGTGAATAAAGTTGT |
| | CCACACCCAAC | | TGGTGTAACCA |
| 1208 | CACTCCCAAAGTCGGCTTCGTCAGTCTTG | 1539 | CCCCTAGTATAGGATGGGTTTCGTTAGG |
| | GATGCCCCAAGGCGCTGGTCGACTCCGAG | | GTGCCCCAACGAATAGAAAAGTAAACTA |
| | CGCATCCTCA | | GCTTTCAGCG |
| 1209 | GGTGAGGATGCGCTCGGAGTCGACCAGCG | 1540 | CTTAAAGATTGAGTTTACTTTTGCAGTC |
| | CCTTGGGGCATCCAAGACTGACGAAGCCG | | ATTGGGGCACCCTAACGAAACCCATCCT |
| | ACTTTGGGAG | | ATACTAGGGG |
| 1210 | CACTCCCAAAGTCGGCTTCGTCAGTCTTG | 1541 | CCCCTAGTATAGGATGGGTTTCGTTAGG |
| | GATGCCCCAAGGCGCTGGTCGACTCCGAG | | GTGCCCCAACGAATAGAAAAGTAAACCA |
| | CGCATCCTCA | | GTTTTCAGCG |
| 1211 | GGTTAAGTGTATGGATATGTTCCCAAATA | 1542 | ACTCAAATGACATTCATTCTGTCCTCTC |
| | CTCCACATTGTGAGACGTGCGTACTTTTG | | AAGCCACGTTGAGTGCGTAGTATTGATG |
| | TCCCACAAAA | | TCAAGGGTTG |
| 1212 | AGCTTTCATTGCGCGACGGATGGGCTATA | 1543 | TTTTTATATAATATAGTGTTTTTGTTAA |
| | GGTACACATCAGGATACAGTAACATTGAA | | GTACACATCACTATATTTGACAAAAAGT |
| | AAAGGAACTG | | CTATAAATAA |
| 1213 | CGCATGTTCGCGGCCGGCACGCTGGTCAC | 1544 | GCCCTGTTAATATGTATATTGGCTAACG |
| | GCTCGGCAACCCGAAGATCATGCTGTTCT | | CTCGGCAACCCGAACGTTAGCCAATATA |
| | ATCTGGCATTG | | CAAACCATGCT |
| 1214 | CGCATGTTCGCGGCCGGCACGCTGGTCAC | 1545 | GCCCTGTTAATATGTATATCGGCTAACG |
| | GCTCGGCAACCCGAAGATCATGCTGTTCT | | CTCGGCAACCCGAACGTTAGCCAATATA |
| | ATCTGGCGTTG | | CAAACCATGCT |
| 1215 | GGGTGGAAATAATATAAAAGGTGGCCTTA | 1546 | AAATTTATAGTGAGGGTTTGTCATAGAC |
| | TAGGTCCTGGAGTTCACGCTTCACATGGT | | AAGACCTCCAATAAGATACAAGAACACA |
| | ATGGAGAGAAC | | ACGGCTTAAAA |
| 1216 | TTTTCCCCCGAAAATCTTTAACACCACTA | 1547 | TTATTTTGGTAGTTTATAGAAGTAATTT |
| | TCTGTTGATGTCCCAGCTCCTCCAAAAAA | | CAGTTGATATTCACTCCATTAACTACCA |
| | AACTAAATAT | | AAATAAAAAA |
| 1217 | TATCTTTTAACTGCAAGAGTACTACGGTT | 1548 | TCCACACGTGTAAGCAGTCCTACACACT |
| | TCCACGTGAGCTGTTTGCGGGAACATATC | | CGATGTGCGTTGAGAGTACACTCTGTAT |
| | GACGGGTTGCA | | CTTCCTACTAT |
| 1218 | ATCTTTTAACTGCAAAAGTACTACGGTCT | 1549 | TTACCCTAGACATCAATGCTACCAACTC |
| | CTACATGAGCTGTTTGCGGGAACATATCG | | AACATGGGACGAGTTGATAGAATTGATG |
| | ACTGGTTGCA | | TATTTGCGAT |
| 1219 | TAAGGGCATGGACATGTTTCCTCATACAC | 1550 | GAAATGACGTACTTTTCATTTCCTCGTG |
| | CTCATGTGGAAACTGTAGTTAAGCTAAGC | | CCATGTGGAGACGGTGGTATTGATGTCA |
| | AAATAATATC | | AGGGCGGAGA |
| 1220 | GCTGGTGGTGGATATCGGCGGTGGTACGA | 1551 | TCCATTAACTGTGGTGTACATCATAACA |
| | CTGACTGTTCATTGCTGCTGATGGGACCG | | TAACTGTTCGTAGTCATGCAAGAATGTA |
| | CAGTGGCGTTC | | CACCGCAGTAA |
| 1221 | ATAATCATCAAAGAGTTTAGGATTATCAA | 1552 | TACTTTAATTTTAGGTTAATGGTCCATT |
| | ATTCACTATGATACGCCCTTCCGAAAGCT | | TCCTCTAGTAAATGTTATATTAACCCAA |
| | GATACTAACGA | | AAAAAAGAGTC |
| 1222 | ACCAGCTGTAACTTTTTCGGATCAAGCTA | 1553 | CACATTATTTAGTTCCTCGTTTTCTCTC |
| | TGAGGGACGCAAAGAGGGAACTAAACACT | | GCTGGACGGAGAATAAATGAGAAACTAA |
| | TAATTGGTGTT | | AATACAAATAA |
| 1223 | AACAATCTGCAAACATGTATGGCGGTACA | 1554 | ATTAATTTTGTACGGAAGTAGATACTAT |
| | TGTATCAACATTGGTTGTATTCCTACAAA | | CTTTCAATATCCATGTTACTTAGTGCCA |
| | GACACTCATT | | TACAAAAACC |
| 1224 | AGGGCCTGGCTGCTGAACTCGGGCGTCTC | 1555 | TCGCGGCCCACTTGCTTTACACGTCTCG |
| | GTCGAGGAAGAGGACGCCCCGGTGGGACA | | TCCAGGAACGAGACGTATAAAACAAGTG |
| | GGGACACCGCG | | GCTACGGCCAG |
| 1225 | ACAATCAACAAAGATGTATGGTGGTACAT | 1556 | TAACGTATGTACGGAAGTATAGACACCT |
| | GCATTAATATCGGATGTATACCTACTAAA | | GATTAATATTTAATGTGTATACTTCCGT |
| | ACATTAATTC | | ATTTTTTATA |
| 1226 | ATGGCTGTTGCGTTGATAGCGCCAAGCGT | 1557 | GTTTTTTTGTTTGCGTTAAATGGAATTA |
| | TACTAGTACGGCATATGCAGTAGAAACAA | | TCCAGTAGGACATTTCCTAAAAGTGGCT |
| | CGAGTCAACA | | AATTTTTTGT |
| 1227 | TATCTTTTAACTGCAAGAGTACTACGGTT | 1558 | TCTTGGCGAGTGAGCAGACCTATACACT |
| | TCCACGTGAGCTGTTTGCGGGAACATATC | | CGATGTGCGTTGACTGTCTACTTAGTAT |
| | GACGGGTTGCA | | CTTCCTACTAT |
| 1228 | ATTAACAAGCACTTTAGATGGAATACAGC | 1559 | GCATAAATATATGGAAGTACACACACTA |
| | CATGGTTTATGCATGTACCGCCATAGCTT | | TACATTGGTTAATTGTGCATACTTCCAT |
| | TCTGTAAATT | | AAAATATTAA |
| 1229 | GACCACAATCCGCGTGTGGGCTTTGTATC | 1560 | GAAGCCGTATAGTATAGGAATGGTGTCG |
| | CCTTGGGTGCCCCAAGGCACTCGTCGATT | | CTTGGGTGCCCGAGTGATGCTTAAAATA |
| | CGGAGCAGATC | | CACTCGGTGCT |
| 1230 | TTCGACGAATGATGCTTTAGGGCTGAATG | 1561 | TTCATTAGCTTTGTTATCACCCTGTTGG |
| | GAGTAAACCTCATGCGCCTAATGGCTACA | | TAACAATCTAATTACACCAACAAGGTGA |
| | AAAAACATCT | | CAACAAAGCA |
| 1231 | CAAAAATTGCAGTGCGTTCAGCGATGACA | 1562 | TTTCTGCATTGTCCTATTATAATTATGA |
| | GGACATTTGATCGCTTCGACGATGCATAC | | GCCATTTGGTCATTATAATAGACCTATA |
| | GAAAGACGCT | | CACATAAACA |
| 1232 | AATTTTCTTGTCGATTGGCTATTCGACTT | 1563 | TATTCTTAGTGGGGCTTAAGTCAACTTG |
| | GTCATTGGTGTCATGTGATGGAGAGAGAA | | TCATTGGTGTCATGTTTTCTTAAGCCTC |
| | TCTTTTGAGG | | AAAATAAAAA |
| 1233 | TTTTAAAATGATTAAAGGCGGCGTTCCAA | 1564 | CTATTAATTGGGGGTATGTCTTACTTAT |
| | TAAGCGTACCCAAGCCCCCAATAGTGCCG | | TAGCGTACCTATTTCGCACCCCCAATAA |
| | GCATAACCGA | | ACACCCCACC |
| 1234 | GGGTGAGGATGCGCTCGGAATCGACAAGG | 1565 | CATCTACCGCAAAGTATAGGTATTTAAT |
| | GCCTTCGGGCAGCCAAGGCTGACGAAGCC | | CCTTCGGGCACCCCAATGAAACAAACCC |
| | GACTTTGGGG | | TATACTTCTA |
| 1235 | AGCAACCCCCCTGCTGTTGGGCTTAACGT | 1566 | TCAAAAAAGCGTGAGTTTTAGATACCAA |
| | GCTTCTCGATGAAAGTGATACTGAGCCTG | | ACATTCTAAAAGCGTATCTAAAACTCTC |
| | AGAAATTAGA | | ATTCAATAGG |
| 1236 | CCATCATAAGATGCCTTTTTACCGACGAG | 1567 | AAAGCATTATTTAGGTACTACAACTAGT |
| | TATAGTTGTACATGCCATTATCAGTCTCC | | ATAGTTGTACATGAAAAACGCTGTATTT |
| | TTTACAAACG | | TTTTATCCAT |
| 1237 | CCAGATCAGTGCGCCCCCGGCGGTCCAGA | 1568 | AAATCCTCCCTTTTACATCTGTACGGGC |
| | GCAGGAAGCGGACATGGCCCATGCGGAAG | | TTGGAAGCAGGCACGTACGGTTGTAAAA |
| | AGGCCCGCTG | | GGAAATCCTA |
| 1238 | TAACACCAATTAAGTGTTTAGTTCCCTCT | 1569 | TCTTTATTTTTTTGTATCCCATTTCCTC |
| | TTGCGTCCCTCATAGCTTGATCCGAAAAA | | TCCCTCCAACGAGAGAAAACGAGAAACT |
| | GTTACAGCTGG | | AAACAATCTAA |
| 1239 | AACAGTTCCTTTTTCAATGTTACTGTAAC | 1570 | TTATTTATAGACTTTTTGTCAAATATAG |
| | CTGATGTGTACCTATAGCCCATCCGTCGC | | TGATGTGTACTTTACAAAAACACTATTT |
| | GCAATGAAAG | | TATATAAATA |
| 1240 | GTGAATGATTTGGTTTTTAATATTTAAAA | 1571 | TTTAATTTATTCGTATTTACGTTACCTT |
| | AAAGAACAACAAAATGTTCCTGATTAAGT | | CACTACTACTAACTTCACATAAACCCAA |
| | GAAGTCATGT | | ACTTTTTACA |
| 1241 | GTGGATCACCTGGTTTTTCGTGTTCAGAT | 1572 | CTCCTTTTATTAGGGTTTGTGTCATCTA |
| | ACAGGCATACGAAGTGCTCCTGAGACAGA | | CACACATGTAAAGTTTACATAAACCCTA |
| | AAGCGCATATC | | AAAAGATCGAC |
| 1242 | ACTTTTTATATTGCAAAAAATAAATGGCG | 1573 | AGTGTGGTTGTTTTTGTTGGAAGTGTGT |
| | GACGAGGTATCAGGATACCTCATCTGCCA | | ATCAGGTAACAGCATAGTTATTCCGAAC |
| | ATTAAAATTTG | | TTCCAATTAAT |
| 1243 | TAACACCAATTAAGTGTTTAGTTCCCTCT | 1574 | ATGTTCTTTTTTTGTATCTCGTTTCTTC |
| | TTGCGTCCCTCATAGCTTGAACCGAAAAA | | TTCTTCCAACGAGAGAAAACGAGGAACT |
| | GTTACAGCTGG | | AAACAATCTAA |
| 1244 | AGATAAAACACTCTCCAGGAAACCCGGGG | 1575 | TGAGACAAACAGCCATGGCTGGTTCCCG |
| | CGGTTCAGATGGCGCACTCATCACCGGAC | | GATACATACAATTATTTGTTATTGTGCA |
| | TGACCTTTCT | | TCATTCTGGT |
| 1245 | ATATGTTCCCGCAAACAGCTCACGTTGAG | 1576 | TATCCCCTCCTCTCAAAACATGTAGAGA |
| | ACGGTAGTACTTTTGCAGTTAAAAGATAA | | CCGTAGTATTGATGTCAAGGGTAGATAA |
| | ATAAAGGACT | | GTAAGAGTGT |
| 1246 | ATATGTTCCCGCAAACAGCTCACGTTGAG | 1577 | TATCCCCTCCTCTCAAAACATGTAGAGA |
| | ACGGTAGTACTTTTGCAGTTAAAAGATAA | | CCGTAGTATTGATGTCAAGGGTAGATAA |
| | ATAAAGGACT | | GTAAGAGTGT |
| 1247 | AACCAGCTGTAACTTTTTCGGATCAAGCT | 1578 | TTAGCTTATTTAGTACCTCGTTTTCTCT |
| | ATGAGGGACGCAAAGAGGGAACTAAACAC | | CGTTGGAAGAAGAATAAACGAGATACCA |
| | TTAATTGGTGT | | AAAAAGAACAT |
| 1248 | TGTTAACCACATAAACATAAATGGTACAA | 1579 | TAAATTTTAATAGCAGTTGTGTCACTAT |
| | CTAATGTGGCACCTGTACCACCCATAGTT | | TTAGGTCTATCGTGTGACAAAACTAACA |
| | ACCACGAACA | | TACAAAAACC |
| 1249 | AAATGTTCGTTGCAACTATGGGGGGTACC | 1580 | AGTTTTATACATAAAAATAGTGTAACAA |
| | GGTGCTACATTAGTCGTTCCATTTATGTT | | GCACTACCTACCCTGTAACACTACTACC |
| | TATGTGGTTA | | ATTAAAATTT |
| 1250 | ATAATGCAACATAGTCTCCAGTACCACCT | 1581 | AAAAAAAGGCGCTCTTTGATGTAGCGCC |
| | TTATATGCACCAGCAGTTGCTGAAAAATC | | CATATGCTCACTACATGAAAAAGCGATA |
| | TATATTTGTT | | ATTTTAAGTA |
| 1251 | ACCAGCTGTAACTTTTTCGGATCAAGCTA | 1582 | TAGATTGTTTAGTTCCTCGTTTCCTCTC |
| | TGAGGGACGCAAAGAGGGAACTAAACACT | | GTTGGACGGAGAATAAATGAGATACTAA |
| | TAATTGGTGTT | | TCCATAATAAT |
| 1252 | AACCAGCTGTAACTTTTTCGGATCAAGCT | 1583 | TTAGATTGTTTAGTTCCTCGTTTTCTCT |
| | ATGAGGGACGCAAAGAGGGAACTAAACAC | | CGTTGGAAGAAGAAGAAACGAGATACCA |
| | TTAATTGGTGT | | AAAAAGAACAT |
| 1253 | ATGAATTAATGTTTTAGTAGGTATACATC | 1584 | GGTTATTTTTACGGAAGTATACACATTA |
| | CGATATTAATGCATGTACCACCATACATC | | AATATTAATCAGGTGTCTATACTTCCGT |
| | TTTGTTGATT | | ACATATGTTA |
| 1254 | AGCTGCGCGCGCAGTATTTCTCGAAGGAG | 1585 | ATGACTTCGATAGTTAATTATGAAACAC |
| | CCCATGGATCCGGACGTATCCATCATGGC | | TCTTGGATATAGGTGCATCAAAATTAAC |
| | GATAATGACC | | TAAAGGAAAA |
| 1255 | TCATCACTACTTAATATATCCATAAGAGA | 1586 | TGCGTTAGGTGTATATCATGCCTAGCGC |
| | AATTTCATTTCCTTCTTTATCTACTCCTA | | AATTCATTACATCATACATGTTGTACAC |
| | TAGGATCTTG | | CTACTTTAAA |
| 1256 | AACCAGCTGTAACTTTTTCGGTTCAAGCT | 1587 | TTAGCTTGTTTAGTACCTCGATTTCTCT |
| | ATGAGGGACGCAAAGAGGGAACTAAACAC | | CGTTGGAGGGAGAAGAAACGGGATACCA |
| | TTAATTGGTGT | | AAAATAAAGAC |
| 1257 | AACCAGCTGTAACTTTTTCGGATCAAGCT | 1588 | TCAACTGGTTTAGTGCCTCATTTCCTCT |
| | ATGAGGGACGCAAAGAGGGAACTAAACAC | | CGTTGGAAGAAGAAGAAACGAGATACCA |
| | TTAATTGGTGT | | AAAAAAGAACA |
| 1258 | ATGAAGGACTTGATTTTTAGTATTGAGAT | 1589 | AGAATTTTATTAGTATTTATGTCAGGTT |
| | AAAGACAAACGAAATTTTCCTGTTGTAAA | | TAAGCATGTAAACATAACATAAACACAA |
| | AACCTCATAT | | AAAATCTTAT |
| 1259 | TCCCCGTGTCGGCGGTTCGATTCCGTCCC | 1590 | TATGTGGGTTTGGTTTTCTGTTAAACTA |
| | TGGGCACCATGAATACGACGAAAAGGCTC | | CACCACCAAAATTCAGCGCCCAACTGTT |
| | ACCTCCGGGTG | | CTCAGTTGGGC |
| 1260 | TCCCCGTGTCGGCGGTTCGATTCCGTCCC | 1591 | TATGTGGGTTTGGTTTTCTGTTAAACTA |
| | TGGGCACCATGAATACGACGAAAAGGCTC | | CACCACCAAAATTCAGCGCCCAACTGTT |
| | ACCTCCGGGTG | | CTCAGTTGGGC |
| 1261 | AACCAGCTGTAACTTTTTCGGATCAAGCT | 1592 | TTAGATTGTTTAGTATCTCGTTATCTCT |
| | ATGAGGGACGCAAAGAGGGAACTAAACAC | | CGTTGGAGGGAGAAGAAACGGGATACCA |
| | TTAATTGGTGT | | AAAATAAAGAC |
| 1262 | GGTGAGGATGCGCTCGGAGTCGACCAGCG | 1593 | CGCTGAAAGCTAGTTTACTTTTCTATTC |
| | CCTTGGGGCATCCAAGACTGACGAAGCCG | | GTTGGGGCACCCTAACGAAACCCATCCT |
| | ACTTTGGGAG | | ATACTAGGGG |
| 1263 | GAGTTCTCTCCATACCATGCGAAGCGTGA | 1594 | ATTCTTTAAAAAGAGTTCTCGTATTTTA |
| | ACTCCAGGACCTATAAGGCCACCTTTTAT | | TTGGAGGTCTTGTCTATGACATACCCTC |
| | ATTATTTCCAC | | ACTATAAATTT |
| 1264 | GAAAGTTTTTCTGAATCCTCTTCATTCAT | 1595 | TTCTCTAATCTTCTTTATTTCTACATAC |
| | TTGGCAACCCCAGGTTTCTATGAAAAATT | | GGTCAACCGTATGTAGAAATAAAGAAGT |
| | CACCTATAACA | | ATTGAGTAGTA |
| 1265 | AGCCTCTGTGCCAAGTATATCTAAAAGAC | 1596 | TAGAAAATAACATATAAAAAGTAGTGTT |
| | TTATTTCATTACCTTCTTTATCTGTTCCG | | TATTTCATTACACACTACTCTTTATATG |
| | ATAGGGTCTT | | TTATTGGTAT |
| 1266 | AGGCAGATCACCTGTAACCCTTCGATTAT | 1597 | AGGCCAGAGCAGCGTCTGGCCTTTAAAT |
| | TCTTGGTGGAGCGGAGGAGGATCGAACTC | | AATGGTGGTGGAATGGCGACGAAATAAA |
| | CCGACCTTCG | | AACCCAAAAT |
| 1267 | GTCTTCTGGACCATGATGCGCCACTTCCG | 1598 | TGTATCTTGATGTACAACATTGCTCTTT |
| | AAATTTCAAAAAGATCAGTGGTCAAACGG | | ATTTTCAAATACAGATTAATGTTGTATA |
| | CTCATTAATTT | | AAGTAACCCTG |
| 1268 | TATGCAACCCGTCGATATGTTCCCGCAAA | 1599 | ATAGTAGGAAGATACTAAGTAGACAGTC |
| | CAGCTCACGTGGAAACCGTAGTACTCTTG | | AACGCACATCGAGTGTGTAGGACTGCTT |
| | CAGTTAAAAGA | | ACACGTGTGGA |
| 1269 | GTTAACAAGCACTTTAGACGGAATACAGC | 1600 | ACATAAATATATGGAAGTACACACACTA |
| | CATGGTTTATGCATGTACCGCCATAGCTT | | TACATTGGTTGATTGTGCATACTTCCAT |
| | TCTGTAAACT | | AAAATATTAA |
| 1270 | GAATGATGCGTTGGGGCTTAATGGAGTAA | 1601 | TATATTGTCATCACCCTGTTGGCGTCAA |
| | ATCTAATGCGCCTAATGGCTACAAAAGAC | | CCTAATTACACCAACAAGGTGACGACAA |
| | ATCTACTTCG | | AGCATAAACG |
| 1271 | GTATTATTAGGGGTGTTTGCAATCGGGGC | 1602 | TACATATTTTCATTATAATTTAAAGACG |
| | ACCAGGAGTCCCTGGGGGGACAGTAATGG | | GTAGGAGTACGAGGTGTCTTTAAATAGT |
| | CATCATTAGG | | TATGAAATTA |
| 1272 | GAAGAGCACCGAGCGCAGGAAGAGCGTGT | 1603 | GGTCAGGCGGCACCTAGGGGGGTGGTTA |
| | ACTGCTCCCACGCCGTCCACTCCGTGATG | | ACGCTCCCATGAGCGTTGCGCACACCCT |
| | CGCCGGTCCGA | | AATGTTGCCTC |
| 1273 | CAGCCGGCTGATTTATTTCCAAATACGCA | 1604 | TCCATAATATGGGTAAGACCTATCACCA |
| | TCACGTGGAGTGCGTAGTGTTGCTACAAC | | CACGTGGAGTGTGTTGCTCTGCTTGTAA |
| | GAAGCAACGGG | | AAGCTTAGAAA |
| 1274 | CAGCCGACTGATTTGTTTCCGAATACGCA | 1605 | ATATGACATCAATGCCATCAACTCGAGC |
| | TCACGTGGAGTGCGTAGTGTTGCTACAAC | | CACGTGGAGTGTGTGGTTCTGCTCGTAA |
| | GAAGCAACGGG | | AAGCCTAGAAA |
| 1275 | AACCAGCTGTAACTTTTTCGGATCAAGCT | 1606 | TTAGATTGTTTAGTTCCTCGTTTTCTCT |
| | ATGAGGGACGCAAAGAGGGAACTAAACAC | | CGTTGGAGGGAGAAGAAACGGGATACCA |
| | TTAATTGGTGT | | AAAATAAAGAC |
| 1276 | AGTTCAGCCCGTGGATTTGTTTCCAATGA | 1607 | TCGTTCCATAATATGGGTAAGACCTATC |
| | CGCATCATGTGGAGTGCATAGCGTTGATA | | ACCACACATCGAGTGTGTGGTTCTGCTC |
| | CAAAGAGTGA | | GTAAAAGCCT |
| 1277 | CGGGCAAATTGCTGCCATATGGACCGGAG | 1608 | CTATTTATTAGATGTCTAAACAGTGCAT |
| | GCGGGACTTTAATTCCTTGGGCGCTTATT | | TACTACTCTACAACCTATATTAGACATC |
| | CCTGCCGCTGC | | TTATAAAAAGT |
| 1278 | GTAACACCAATTAAGTGTTTAGTTCCCTC | 1609 | TATTTATAATTTTAGTTTCTCGATTCGT |
| | TTTGCGTCCCTCATAGCTTGATCCGAAAA | | CTCCGTCCAGCGAGAGATAACGAGGTAC |
| | AGTTACAGCTG | | TAAATAATCTA |
| 1279 | TCTAACTCACGACACGTTGTACTCTTACC | 1610 | CAGTTTTTATTTTATGCCTTAATTATAC |
| | AACCGCACTTGCTCCCTCAAACGCTATAA | | ACCGCACTTGCGGTATGTCAATATGGCA |
| | TCCCCATAGTT | | AAAAGCTATTC |
| 1280 | AGGCAGATCACCTGTAACCCTTCGATTAT | 1611 | AGGCCAGAGCAGCGTCTGGCCTTTAAAT |
| | TCTTGGTGGAGCGGAGGAGGATCGAACTC | | AATGGTGGTGGAATGGCGACGAAATAAA |
| | CCGACCTTCG | | AACCCAAAAT |
| 1281 | AGCAGGATGGAGATAACGAGCATGACGAC | 1612 | AAACAAAAATAAGGGGTTATTACCCCTA |
| | TAACATTTCTATCAGTGTAAATCCCTTTT | | TTTATTTCAATAAATATGGGTAATAACC |
| | CATTCACAGTT | | CTTAAATGATT |
| 1282 | CTTGTGGATCACCTGGTTTTTCGTGTTCA | 1613 | TGTCTCTTTTTATTAGGGTTTATATCAA |
| | GATACACACATACGAAGTGCTCCTGAGAG | | CTACACACATGTAAAGTAGACATAAACA |
| | AGAAAGCGCAT | | GCAAAAATTTG |
| 1283 | ATATCCCAAATGGAAAAGTTGTTAAACCG | 1614 | AAAAATTTAGTTGGTTATTGGTTACTGT |
| | TGTATAACGATACCAATCCCCCAACCTCC | | AACAAATCTTACGGTAACCAATAACCAA |
| | AAGTGGATAT | | CTTTAAAACT |
| 1284 | TTTAAATTTTGTCCTTTCTTCCCGCTATA | 1615 | TTTTTATTTTTATCCCCTAATTATACAT |
| | CCCGCTTGGCATTGTAAAAGATAAATAGT | | GGGATTCCTCATATGTCAATAAGGATAA |
| | TCGCCCACTC | | AAATATTATT |
| 1285 | ATGGCTGTTGCGTTGATAGCGCCAAGCGT | 1616 | GTTTTTTTGTTTGCGTTAAATGGAATTA |
| | TACTAGTACGGCATATGCAGTAGAAACAA | | TCCAGTAGGACAGTTCCTAAAAGTGGCT |
| | CGAGTCAACA | | AATTTTTTGT |
| 1286 | CCAAATATTAAATTCTGCAGTAGGCGTCC | 1617 | AAAGTTTAGATGGGGTTTGTGGGTAGAG |
| | AATTTCCAAAGGTTCCTCCACCCATAATT | | CCTCCCGAATAACACACCAAAACCCCCA |
| | GTTATAGAAT | | CATATGCCAC |
| 1287 | CATTTTTACCTTGCTCTTCTCTCGAATTT | 1618 | AGTTTTATTTTTGTCTGTATAGGCTGTC |
| | CAGCATCTGCATGGCGCATAACATATTTA | | CGCATCTGCGGTATGCTTATAGGGACAA |
| | TGCGCTACAG | | AAATTATAAA |
| 1288 | TTTGCGAGACTACGGATCTGGATCTCGTC | 1619 | GCTAACAGATCGGCATATGAGTGCTATC |
| | CCACTGCTGGCGCGGTCCCGCGATATCGC | | TACTGCTGGCAGTGAACTGTACTCAGAC |
| | GCCGCAGGTAC | | GCAAATAAGCA |
| 1289 | AGAAAAGCACGCTGATAATCAGCAAGACC | 1620 | AATTGGAAAATATAAATAATTTTAGTAA |
| | ACCAACATTTCCACAAGTGTAAAAGCTTT | | CCTACATTTCAATCAAGGATAGTAAAAC |
| | AACCTTCGCT | | TCTCACTCTT |
| 1290 | ACACCAGAAATCAAGGAGTCTTACCAGTA | 1621 | TTTTATCAAAAATTTTACTATCCTTGAT |
| | TGGAAATGAAAATACAAGCTTCTTTACCA | | TGAGATGTAGGTTACTAAAATTATTTAT |
| | GTATGATTCCG | | ATTTTCCACTT |
| 1291 | ATGTACGAGTACTTTAGAGGGTATACAGC | 1622 | TTATTTTATTATGGAAGTTTGTACACTT |
| | CGTGGTTTATGCATGTGCCGCCAAAGTTG | | AACATTGCAAGACTGTACATACTTCCAT |
| | TCTGAGGATT | | AGTTTATTAA |
| 1292 | AACAATCTGCAAACATGTATGGCGGTACA | 1623 | ATTAATTTTGTACGGAAGTAGATACTAT |
| | TGTATCAACATTGGTTGTATTCCTACAAA | | CTTTCAATATAGAACGTTTATAGTTCCA |
| | GACACTCATT | | TACAAAAATA |
| 1293 | TGTAACACTTCATTTTTGACGTTCAGAAA | 1624 | TAAAATAGTATGTATTTATGTAAGTTTA |
| | CAGCACGACGAAATGTTCCTGGTTCAATG | | ACCACGACCAACCTTACATAAATGGTAA |
| | ACGACATATCT | | CTATTATATAT |
| 1294 | GCTTCTGGACGCGGGTTCGATTCCCGCCG | 1625 | CCCGACAGTTGATGACAGGGTGCGACCC |
| | CCTCCACCACCCAACACCCCGGAAAGCCC | | CACCACCAATATCCGAACCCTAACCGCT |
| | TTGTTTTACA | | CTCGGTTGGG |
| 1295 | GCTTCTGGACGCGGGTTCGATTCCCGCCG | 1626 | CCCGACAGTTGATGACAGGGTGCGACCC |
| | CCTCCACCACCCAACACCCCGGAAAGCCC | | CACCACCAATATCCGAACCCTAACCGCT |
| | TTGTTTTACA | | CTCGGTTGGG |
| 1296 | GTAACACCAATTAAGTGTTTAGTTCCCTC | 1627 | TATTTATAATTTTAGTTTCTCGATTCGT |
| | TTTGCGTCCCTCATAGCTTGATCCGAAAA | | CTCCGTCCAGAGAGAGAAATTGAGGTAC |
| | AGTTACAGCTG | | TAAACAACGTA |
| 1297 | ACCGTAAAATAACATTTCTGTTTTTCCAG | 1628 | GTAATTATTTTATGTATTCATTTCCGGC |
| | CCCCGCACACAGCCCAAATAAAAAAAGAT | | TATTCAAGTAGCTAGTCTTGAATACCGA |
| | TTTTTCTGCT | | AAAAAAATTC |
| 1298 | GAATGATGCGTTGGGGCTTAATGGAGTAA | 1629 | TATATTGTCATCACCCTGTTGGCGTCAA |
| | ATCTAATGCGCCTAATGGCTACAAAAGAC | | CCTAATTACACCAACAAGGTGACGACAA |
| | ATCTACTTTG | | AGCGCGAACG |
| 1299 | GAAACTATGGGGATTATAGCGTTTGAGGG | 1630 | GAATAACTTTTTGCCGTATTGACATACC |
| | AGCAAGTGCGGTTGGTAAGAGTAGCACGT | | GCAAGTGCGGTGTATAATTAAGGCATAA |
| | GTCGTGAATTA | | AATAAAAAACG |
| 1300 | TTCGGACGCGGGTTCAACTCCCGCCAGCT | 1631 | GAATGAATAGCTAATTACAGGGACGCCA |
| | CCACCAAATATTGATGTACTGAAGTTCAG | | GCCCAAATAAAACAAGGGGTTACGTGAA |
| | TAAAGTCTACT | | AACGTAGCCCC |
| 1301 | AATTTTTAAAAAAAGTCGACAAGCATTTA | 1632 | TAATAGAAAGAAAAATATATTTATTATA |
| | CTCTAATTGAAGCAGCAATTGTGCTTTTC | | TCTAATTGAAACGGCTTATAGTCATTAT |
| | ATTATTAGTT | | GTTTATTTTG |
| 1302 | AGAGAAGTTGCCGGAAGCATGGTTCTAGT | 1633 | TAGATAGAGTTTATGGATTATAAGAGGT |
| | TTCTTTGGAAGAAAAGAAGGAACGAAGGA | | TTATTGGGCAAAACCTCTTGAAATACAT |
| | GTTAACGCGT | | AAAAAGAGTT |
| 1303 | CACCTGGCGTGGCGAAGTGCGCAGTCTGG | 1634 | AAGAGATTCACCAAGACTTTTAGATTGA |
| | AAGCACTAAATAGCTGCGCGGAATAGTAG | | CCACCTAGTACGTTGGCAGTCACCTGAA |
| | ATCACTTTGAG | | CGTGGGTTGAT |
| 1304 | ATAACGCATACATTGTTGTTGTTTTTCCA | 1635 | ATCAATAACGGTTGTATTTGTAGAACTT |
| | GATCCAGTTGGTCCTGTAAATATAAGCAA | | GACCAGTTTTTTTAGTAACATAAATACA |
| | TCCATGTGAG | | ACTCCGAATA |
| 1305 | TATGTTCAGGTTTGATCATTTTCCAAAAA | 1636 | ACTCAAATGACATCAATTCTGTCCTCTC |
| | CGTATCAAAGCGTGTGTGTTCAACGTTTT | | AAGACATGTGGAGTGTGTTGTCTTGATG |
| | TTTCTTTTCC | | TCAAGGGTGG |
| 1306 | TATGTTCAGGTTTGATCATTTTCCAAAAA | 1637 | ACTCAAATGACATCAATTCTGTCCTCTC |
| | CGTATCAAAGCGTGTGTGTTCAACGTTTT | | AAGACATGTGGAGTGTGTTGTCTTGATG |
| | TTTCTTTTCC | | TCAAGGGTGG |
| 1307 | TATGCAACCCGTCGATATGTTCCCGCAAA | 1638 | ATAGTAGGAAGATACTAAGTAGACAGTC |
| | CAGCTCACGTGGAAACCGTAGTACTCTTG | | AACGCACATCGAGTGTGTAGGACTGCTT |
| | CAGTTAAAAGA | | ACACGTGTGGA |
| 1308 | TAACACCAATTAAGTGTTTAGTTCCCTCT | 1639 | GTCTTTATTTTTGGTATCCCGTTTCTTC |
| | TTGCGTCCCTCATAGCTTGAACCGAAAAA | | TCCCTCCAACGAGAGAAATCGAGGTACT |
| | GTTACAGCTGG | | AAACAAGCTAA |
| 1309 | GTAACACCAATTAAGTGTTTAGTTCCCTC | 1640 | ATTATTATGGATTAGTATCTCATTTATT |
| | TTTGCGTCCCTCATAGCTTGATCCGAAAA | | CTCCGTCCAGCGAGAGATAACGAGGTAC |
| | AGTTACAGCTG | | TAAATAATCTA |
| 1310 | GCTGGTGGTGGATATCGGCGGTGGTACGA | 1641 | TCCATTAACTGTGGTGTACATCATAACA |
| | CTGACTGTTCATTGCTGCTGATGGGGCCG | | TAACTGTTCGTAGTCATGCAATAATGTA |
| | CAGTGGCGTTC | | CACCGCAGTAA |
| 1311 | TATGCAACCAGTCGATATGTTCCCGCAAA | 1642 | ATAGTAGGAAGATACAGAGTGTACTCTC |
| | CAGCTCATGTAGAGACCGTAGTACTTTTG | | AACGCACATCGAGTGTGTAGGACTGCTT |
| | CAGTTAAAAG | | ACACGTGTGG |
| 1312 | AACCAGCTGTAACTTTTTCGGATCAAGCT | 1643 | TTAGCTTGTTTAGTACCTCGATTTCTCT |
| | ATGAGGGACGCAAAGAGGGAACTAAACAT | | CGTTGGAGGGAGAAGAAACGGGATACCA |
| | TTAATTGGTGT | | AAAATAAAGAC |
| 1313 | AACCAGCTGTAACTTTTTCGGATCAAGTT | 1644 | TTAGATTATTTAGTACCTCGTTATCTCT |
| | ATGATGGACGTAAAGAGGGAACAAAGCAC | | CGCTGGAAGAAGAAGAAACGAGAAACTA |
| | CTAATAGGTGT | | AAATTATAAAT |
| 1314 | TAACACCAATTAAGTGTTTAGTTCCCTCT | 1645 | GTCTTTATTTTTGGTATCCCGTTTCTTC |
| | TTGCGTCCCTCATAGCTTGAACCGAAAAA | | TCCCTCCAACGAGAGATAACGAGATACT |
| | GTTACAGCTGG | | AAACAATCTAA |
| 1315 | ATAATCATCAAAGATTTTAGGATTATCAA | 1646 | TACTTTAATTTTGGGTTAATGGTCCATT |
| | ATTCACTATGATACGCCCTTCCGAAAGCT | | TCCTCTAGTAAATGTATTATTAACCCAA |
| | GATACTAACGA | | AAAAAGAGTCT |
| 1316 | CATCTTTACTTTGCTCTTTTCTCGAATTT | 1647 | AGTTTTATTTTTGTCTATATAGGCTGTC |
| | CAGCATCTGCGTGTCTCATAACGTATTTA | | GGCATCTGCGGTATGCTTATAGGGACAA |
| | TGCGCTACAG | | AAATTATAAA |
| 1317 | CTGTTTCAACAAATGATGCTCTTGGCCTT | 1648 | AAAAATAAATATCTTTGTCGCCATCGTG |
| | AATGGTGTAAACCTTATGCGTTTAATGGC | | TTGGTGTAAACCTAATTACACCAACAAG |
| | GACAAAACATA | | GTGACAACAAA |
| 1318 | AGCTAAGTGTCCTAATTGGCCCCCGATCC | 1649 | TACATAATTTCGTATATTAGGTATAACC |
| | CGGTTTCAATAGTTTGGGGAATCTTTGTA | | AGTTTCAATTGGAAATACCTAATATACG |
| | AGTGGTAAGC | | AAAAAGGTGT |
| 1319 | CGGCCTTCCACTTACAAAAATTCCGCAGA | 1650 | CGCCTTTTTTCGTATATTAGGTATTTCC |
| | CAATTGAAACCGGGATCGGGGGCCAATTA | | AATTGAAACTGGTTATACCTAATATACG |
| | GGACACTTAG | | AAAATATGCA |
| 1320 | GTAGATGTTTTTTGTTGCCATTAGGCGCA | 1651 | CGCTTTGTTGTCACCTTGTTGGTGTAAT |
| | TGAGGTTTACTCCATTAAGCCCTAAAGCA | | TAGATTGTTACCAACAGGGTGATAACAA |
| | TCATTCGTCG | | AGCTAATGAA |
| 1321 | AATATGTTTTGTCGCCATTAAACGCATAA | 1652 | TTTGTCGTCACCTTGTTGGTGTAATTAG |
| | GGTTTACACCATTAAGGCCAAGAGCATCA | | GTTTACACCAACATGATGACAACGAAGA |
| | TTTGTTGAAAC | | TATTTACTTTT |
| 1322 | AATATGTTTTGTCGCCATTAAACGCATAA | 1653 | TTTGTCGTCATCTTGTTGGTGTAATTAG |
| | GGTTTACACCATTAAGGCCAAGAGCATCA | | GTTTACACCAACTTGATGACGACAAAAA |
| | TTTGTTGAAAC | | TATTTATTTTT |
| 1323 | CGTCGTTAGTATCAGCTTTCGGAAGGGCG | 1654 | AGACTCTTTTTTTGGGTTAATAAAACAT |
| | TATCATAGTGAATTTGATAATCCTAAAAT | | TTACTAGAGGAAATGGACCATTAACCTA |
| | CTTTGATGATT | | AAATTAAAGTA |
| 1324 | GCGCGTGATATTGCGACGTATTTTAATCA | 1655 | ACAATACATTTTACTTCAATGTATAGGT |
| | TACATTCGGCACGACATTTACACTTCCGA | | ACATTCGGCACAGCGAGTTTATCTATAA |
| | AGTATGTCAT | | GTTGAAGTAA |
| 1325 | GTTTTTTGTTGCCATTAGGCGCATGAGGT | 1656 | GTCGTCACCTTGTTGGTGTAATTAGGTT |
| | TGACGCCATTAAGCCCTAGAGCATCATTC | | GACTCCAACAGGGTGATGACAATATAAA |
| | GTCGAAACAGC | | CATTTCTTTTT |
| 1326 | ATTGATTCTACAACAGAAGTTGGCATACT | 1657 | CGCTCCTTTAATTTTGCTTAAAGGAGCA |
| | AGAAACTAGTACTTTAAGAGCACCAAAAA | | AAGACTAGTATCTTATTTATCTTAAGCT |
| | TAAATAATGTA | | AAAATTAAAAT |
| 1327 | CATCTTTACTTTGCTCTTCTCTCGAATTT | 1658 | AGTTTAATTTTTGTCTATATTGGCTGTC |
| | CAGCATCTGCATGGCGCATCACATATTTA | | TGCATCTGCGGTATACTTATAGGGACAA |
| | TGCGCTACAG | | AAATTATAAA |
| 1328 | AAAATTAACAAGCTAATAATGAACAAGAC | 1659 | TTTTATACCTTTTTGAATATATTTAGAG |
| | AATCGTCATTTCCACCAGGGTAAAGCCCT | | ATCGTCATTTCAATAGCACTCCCCAAAT |
| | TGGCCACCCGT | | CTTTTTAATAG |
| 1329 | TTTGTTGACTCGTTGTTTCTACTGCATAT | 1660 | ACAAAAAATTAGCCACTTTTAGGAACTG |
| | GCCGTACTAGTAACGCTTGGCGCTATCAA | | TCCTACTGGATAATTCCATTTAACGCAA |
| | CGCAACAGCC | | ACAAAAAAAC |
| 1330 | TAACACCAATTAAGTGTTTAGTTCCCTCT | 1661 | TGTTCTTTTTTTGGTATCTCGTTTCTTC |
| | TTGCGTCCCTCATAGCTTGATCCGAAAAA | | TTCTTCCAACGAGAGAAAACGAGGTACT |
| | GTTACAGCTGG | | AAATAAACTAA |
| 1331 | GTCTTCTGGACCATGATGCGCCACTTCCG | 1662 | TGTATCTTGATGTACAACATTGCTCTTT |
| | AAATTTCAAAAAGATCAGTGGTCAAACGG | | ATTTTCAAATACAGAATAATGTTGCATA |
| | CTCATTAATTT | | AAATAGCCCTG |
| 1332 | TAACACCAATTAAGTGTTTAGTTCCCTCT | 1663 | ATGTTCTTTTTTGGTATCTCGTTTCTTC |
| | TTGCGTCCCTCATAGCTTGATCCGAAAAA | | TTCTTCCAGCGAGAGATAACGAGGTACT |
| | GTTACAGCTGG | | AAATAATCTAA |
| 1333 | CGCGACACCAGCCTCGTCGTGGTCCCGCA | 1664 | GGTTTTCTTTGCCCCTTTGCGCGCACAG |
| | GTTCCACGTCAACGCCTGGGGCCTGCCGC | | TCCCACGTATGTGCGCGCAAAGGGGGAA |
| | ACGCGGTGTT | | GGAGGCGGCC |
| 1334 | GTGTCGGCAGCCCTGCAGGTCGGATATCG | 1665 | CTGCATCTACCATGTTCTACAATCTACC |
| | CAGCATCGACACCGCCAAGATCTACGACA | | AGCATCGACACTTCATTGGTAGGACTTG |
| | ACGAGGCGGG | | GTAGAACGGT |
| 1335 | TCCGCAGCAATATCTTCATACAAATCGGC | 1666 | GCGCATTTAGTTTGTGTTTTTAAAAGCA |
| | AATAGGATCTCCTTTTGCCTGGATATAAG | | ATAGGATCTCCTTTTGCTTTTAAAGACA |
| | TGGCAGTGAAT | | TAACAAATAGT |
| 1336 | TATCTTTTAACTGCAAGAGTACTACGGTT | 1667 | TCTTGGCGAGTGAGCAGACCTATACACT |
| | TCCACGTGAGCTGTTTGCGGGAACATATC | | CGATGTGCGTTGACTGTCTACTTAGTAT |
| | GACGGGTTGCA | | CTTCCTACTAT |
| 1337 | ACCAGCTGTAACTTTTTCGGATCAAGCTA | 1668 | TACGTTGTTTAGTACCTCAATTTCTCTC |
| | TGAGGGACGCAAAGAGGGAACTAAACACT | | TCTGGACGGAGACGAATCGAGAAACTAA |
| | TAATTGGTGTT | | AATTATAAATA |
| 1338 | CATTTTTACCTTGCTCTTCTCTCGAATTT | 1669 | AGTTTTATTTTTGTCTGTATAGGCTGTC |
| | CAGCATCTGCATGGCGCATAACATATTTA | | CGCATCTGCGGTATGCTTATAGGGACAA |
| | TGCGCTACAG | | AAATTATAAA |
| 1339 | ACCAGCTGTAACTTTTTCGGATCAAGCTA | 1670 | TAGATTATTTAGTACCTCGTTATCTCTC |
| | TGAGGGACGCAAAGAGGGAACTAAACACT | | GCTGGACGGAGACGAATCGAGAAACTAA |
| | TAATTGGTGTT | | AATTATAAATA |
| 1340 | TATGCAACCCGTCGATATGTTCCCGCAAA | 1671 | ATAGTAGGAAGATACTAAGTAGACAGTC |
| | CAGCTCACGTGGAAACTGTAGTACTCTTG | | AATGCACATCGAGTGTGTAGGTCTGCTT |
| | CAGTTAAAAGA | | ACTCGTGTAGA |
| 1341 | TCGTTTCAATATGTCCGTACATGGAATAA | 1672 | ATCATCCTTATACGTGTTTAGCTATGTA |
| | TAAAGCACCAGAACTTTAGCCATTTCTAA | | AAAGCACCAGTATTCTTGCCTTAACACT |
| | CCACTCCTCG | | CATGGTATTC |
| 1342 | CGAACATCTATAAATTCTGTATTGGTAGA | 1673 | GGTTTTTTTGTGTGTGGTTTTGTATGTT |
| | AACATCACAGGTGCTTTCCCTCCTGGTGA | | AAATCACAATCAAAATGCTAATACCACA |
| | ACAGTACAAC | | CACTACAATA |
| 1343 | ATAGTATTAGCTGGCGGATGTGCAACTGG | 1674 | ATTACAATATTACTTTATTTAGTCTATC |
| | CACATGGTATCGAGCTGGGGAAGGATTAA | | TTTAGGTGGAACTGGACTGAATTAAGTC |
| | TTGGTAGTTGG | | AAAATATAAAC |
| 1344 | CGACAAGGACACCACGCTCGTCGTGGTCC | 1675 | CACCTTTTTTATTTGCCCCTTTAGGCGC |
| | CTCAATTCCACGTGAACGCCTGGGGCCTG | | ACTGTTTCACGTCTGTGAGCCTAAAGGG |
| | CCGCACGCCA | | GCATCCCCAC |
| 1345 | GACGACGTCAAATGAGAAATCTGTTACAC | 1676 | TTTTTACAAAGAGGTATTTAGATACATG |
| | GTGTAACATTAGCAGTTAACCGCCGTTTT | | AGCTACAATGCCTGTATCTAAATACCTC |
| | AAATCGCAAAA | | TAAAGAAAGAC |
| 1346 | CTGTGCCGCCCGAGTGATCTGCGTGCACA | 1677 | AAAGTTTTTTTAGACGTACTAACCAATA |
| | ATCATCCCAGCGGCAGTCCCCAACCTTCG | | TCATCCCAGCGGAAAGTATCAGTTAGGC |
| | CAGGCGGATAT | | ACATAAATTAG |
| 1347 | ATGGCTGTTGCGTTGATAGCGCCAAGCGT | 1678 | GGTTTTTTGTTTGCGTTAAATGGAATTA |
| | TACTAGTACGGCATATGCAGTAGAAACAA | | TCCAGTAGGACAGTTCCTAAAAGTGGCT |
| | CGAGTCAACA | | AATTTTTTGT |
| 1348 | GAATGATGCGTTGGGGCTTAATGGAGTAA | 1679 | TATATTGTCATCACCCTGTTGGCGTCAA |
| | ATCTAATGCGCCTAATGGCTACAAAAGAC | | CCTAATTACACCAACAAGGTGACGACAA |
| | ATCTACTTTG | | AGCACGAACG |
| 1349 | GTCTTCTGGACCATGATGCGCCACTTCCG | 1680 | TGTATCTTGATGTACAACATTGCTCTTT |
| | AAATTTCAAAAAGATCAGTGGTCAAACGG | | ATTTTCAAATACAGATTAATGTTGTATA |
| | CTCATTAATTT | | AAGTAACCCTG |
| 1350 | ATAGAAATAGACCTTTCCACTGGCCAAGG | 1681 | AATTATTACTTGTGTTTTTGTAGTGGTT |
| | AGCTGATAAAACCATGCAACAAGTTTTAA | | GCTGATAAAACTATTACAAATACACAAG |
| | GTAAAAGTGCA | | TATAGAAATAG |
| 1351 | TTGATATGATATTTTATAACGGTTAATAT | 1682 | GGGAAAGTTTTGGGGAAGATTTTACATC |
| | ATTTATAAAACAACGGGCGTGTTATACGC | | ATCATAATAAATATCCTCCGGCATAGCC |
| | CCGTTTCAAT | | GGAGGTTTTT |
| 1352 | AACGTTTGTAAAGGAGACTGATAATGGCA | 1683 | ATGGATAAAAAAATACAGCGTTTTTCAT |
| | TGTACAACTATACTCGTCGGTAAAAAGGC | | GTACAACTATACTAGTTGTAGTGCCTAA |
| | ATCTTATGAT | | ATAATGCTTT |
| 1353 | GATAGTGATCGAATATATTCATGGTATGC | 1684 | TAAAATGTTCCCATTGATTGTGGTGTGT |
| | CGTCCTTTCGTTTTTTAGCACAGGTTAAG | | GTCCTTTCGTATACTATGGGAACATTTT |
| | AGCCGTTCAT | | GATTTAATAC |
| 1354 | CCCGAAGGATGCTCCCCGCTCCACCACCG | 1685 | TGGGGTCTTGCATCCAGCGTGAATGGTT |
| | TTTATGACCCGACCTGTGGATCTGGTTCG | | GTGCGAAACTTTCATGCCACGCTGGATA |
| | CTGTTGATCA | | CAAACGCGCG |
| 1355 | AATGTTTATCGTTACTTTTGGAGGTACGG | 1686 | TTTTTTTACGTGAATGTTTTGTAACTAC |
| | GTGCAACATTGGTCGTCCCGTTCATGTTT | | TACGACCTACCTCGTAACACACCATTCA |
| | ATGTGGATGA | | TCAAAATCTA |
| 1356 | TAACTCACGACACGTTGTGCTCTTACCAA | 1687 | GTTTTTATTTTATGCCTTAATTATACAC |
| | CCGCACTTGCTCCCTCAAACGCTATAATC | | CGCACTTGCAGTATGTCAATATGGCAAA |
| | CCCATAGTTT | | AAGCTATTCT |
| 1357 | ACAATCATCAGATAACTATGGCGGCACGT | 1688 | TTAATTTAGTATGGAAGTATGCACAATT |
| | GCATTAACCACGGTTGTATCCCGTCTAAA | | AACCAATGTTTAGTGTGTATACTTCCAT |
| | GTACTCGTAC | | AAAAATTAAC |
| 1358 | TATGCAACCAGTCGATATGTTCCCGCAAA | 1689 | ATAGTAGGAAGATACTAAGTAGACAGTC |
| | CAGCTCATGTAGAGACCGTAGTACTTTTG | | AACGCACATCGAGTGTGTAGGACTGCTT |
| | CAGTTAAAAG | | ACACGTGTGG |
| 1359 | GCAACCGGCATCAATGTAATACCGATAAT | 1690 | CAAATAATGTAGTACCCAAATTATGTTT |
| | CGTAACAACAGAGCCTGTCACGACCGGCG | | CACACAAGCAACCTTAATCGGGTACTAC |
| | GAAAAAACGA | | TTAATATCTA |
| 1360 | AAGAACACTAATAATCAGCAAAACAACTA | 1691 | TGGAAAATTTGATAAATTTGGTTACGTT |
| | GCATTTCAATCAGCGTAAAAGCTTTTACT | | CATTTCAATCAAGGATAGTGAAATTATT |
| | TTGAGTGTACG | | GCTTTTTCGAA |
| 1361 | GAGAGAGTAGAGTGTTGTTGTCTTGCCAG | 1692 | CTTGTTTTATTAATATTTACGTAACGTT |
| | ACCCAGTTGGACCGGTCAGAATTATTAAT | | ATCAGTTGGTAGCGTTACGTAAATATAA |
| | CCGTGTGCATG | | CTAATTATTTA |
| 1362 | CTTGTAAAACAAGGGCTTTCCGGGGTATT | 1693 | CCCAACCGAGAGCGGTTAGGGTTCGGAT |
| | GGGTGGTGGAGGCGGCGGGAATCGAACCC | | ATTGGTGGTGGGGTCGCACCCTTGTATG |
| | GCGTCCAGAA | | AAACTGACCT |
| 1363 | CTTGTAAAACAAGGGCTTTCCGGGGTATT | 1694 | CCCAACCGAGAGCGGTTAGGGTTCGGAT |
| | GGGTGGTGGAGGCGGCGGGAATCGAACCC | | ATTGGTGGTGGGGTCGCACCCTTGTATG |
| | GCGTCCAGAA | | AAACTGACCT |
| 1364 | CACTCCCAAAGTCGGCTTCGTCAGTCTTG | 1695 | CTCCCAGTGTAGGATTTATATCGCTAGG |
| | GATGCCCCAAGGCGCTGGTCGACTCCGAG | | GTGCCCCAACGAATAGAAAAGTAAACCA |
| | CGCATCCTCA | | GTTTTCAGCG |
| 1365 | CACTCCCAAAGTCGGCTTCGTCAGTCTTG | 1696 | CCCCTAGTATAGGATGGGTTTCGTTAGG |
| | GATGCCCCAAGGCGCTGGTCGACTCCGAG | | GTGCCCCAACGAATAGAAAAGTAAACCA |
| | CGCATCCTCA | | GCTTTCAGCG |
| 1366 | ATGATCTGCTCCGAATCGACGAGTGCCTT | 1697 | AGCGATGAGTATACTTTTGCTATCCTAC |
| | GGGGCACCCAAGGGATACAAAGCCCACAC | | GGGCACCCAAGCGACACCATTCCTATAC |
| | GCGGATTGTGG | | TATACGGCTTC |
| 1367 | GTCTTCTGGACCATGATGCGCCACTTCCG | 1698 | TGTATCTTGATGTACAACATTGCTCTTT |
| | AAATTTCAAAAAGATCAGTGGTCAAACGG | | ATTTTCAAATACAGAATAATGTTGCATA |
| | CTCATTAATTT | | TAATATTACTA |
| 1368 | AAAGCTAAGGTTAAAGCTTTTACATTGAT | 1699 | AAGAGTGAGAGTTTTACTATCCTTGATT |
| | TGAAATGTTGGTGGTCTTGCTGATTATCA | | GAAATGTAGGTTACTAAAATTATTTATA |
| | GCGTGCTTTT | | TTTTCCAATT |
| 1369 | TAGATACACCTGCAATTTGTTGTAATGGC | 1700 | CTTCTAATTTTTGTTTGTATAAGCATAA |
| | ACTTATTTGTATGATTATCAGGCAAAAAA | | CACATTTGAGTGTGTGACGCTTATTACA |
| | GGTTTTAGAAT | | ACATTTTCACC |
| 1370 | TCGTACGCCGGGGAGACGACGTTCGCCGC | 1701 | AGCTCGGGTTCTTCGTGTTTTGCCACGT |
| | GATGTTGACCGAGAGCGTGGCGACGAGGA | | ATGTTGACCGACAGACACGGCAAAACAC |
| | CGGTCACCAGG | | GCAGCGCCTAT |
| 1371 | GGATTTCGTTGCACTGATGGGCGGTACTG | 1702 | TCTTTTTTTATGTATGGTTTGTAACAAT |
| | GCGCGACTTTACTCGTTCCTTATTTATTT | | ATCCACCTACAATGTGCTAAACCATACA |
| | ATATTTCTTT | | TGTTAAAAAT |
| 1372 | AGTACAACCAGTCGATTTATTCCCACAAA | 1703 | ATAGTAGGAAGATACAGAGTGTACTCTC |
| | CACATCATGTGGAATTAGTGGCGCTATTA | | AACGCACATCGAGTGTGTAGGACTGCTT |
| | GCACCTAAGG | | ACACGTGTGG |
| 1373 | AGTACAACCAGTCGATTTATTCCCACAAA | 1704 | ATAGTAGGAAGATACAGAGTGTACTCTC |
| | CACATCATGTGGAATTAGTGGCGCTATTA | | AACGCACATCGAGTGTGTAGGACTGCTT |
| | GCACCTAAGG | | ACACGTGTGG |
| 1374 | ACATAAAAATATAGATTTTCCAGGGCATA | 1705 | CGAAATATCGCAATTACATAAAGCATGT |
| | ATCATGCATGGCTATATGATGTGAATAAA | | ACATGCATGGTTTATAGTATTGCAACCA |
| | ATAGAACCCGA | | TTCTACCAAAT |
| 1375 | GTCTTCTGGACCATGATGCGCCACTTCCG | 1706 | TGTATCTTGATGTACAACATTGCTCTTT |
| | AAATTTCAAAAAGATCAGTGGTCAAACGG | | ATTTTCAAATACAGAATAATGTTGCATA |
| | CTCATTAATTT | | TAATATTACTA |
| 1376 | GGTTAAGTGTATGGATATGTTCCCAAATA | 1707 | TGTTGAATAGGTTGGTCATTGGAGAACC |
| | CGCCACATTGTGAGACTGTAGTTAAACTT | | GAGCCACGTTGAGAGCGTAGTATTGTTG |
| | ATTAGAGAAT | | ACTAAAGCAC |
| 1377 | GGTTAAGTGTATGGATATGTTCCCAAATA | 1708 | TGTTGAATAGGTTGGTCATTGGAGAACC |
| | CGCCACATTGTGAGACTGTAGTTAAACTT | | GAGCCACGTTGAGAGCGTAGTATTGTTG |
| | ATTAGAGAAT | | ACTAAAGCAC |
| 1378 | AAAGCGAATGGCAAGCTCAGGCCACTCGG | 1709 | TTGAGCACTTGTGCAGTTCGCGTTGACC |
| | CATTCCGAGCCTGCGGGATCGGATCGTGC | | GTCCCGACGGTGACTTCATAATGCACCT |
| | AGCGGGCTAT | | CTCACAGTTG |
| 1379 | TAAGAAGAAAGACTCTTTTTTTATTTGGG | 1710 | TGAATTTTTTTCGGTATTCAAGACCAGC |
| | CTGTGTGCGGGGCTGGAAAAACTGAAATG | | TACTTGAATAGCCCGAAATGAATACATA |
| | CTATTTTACG | | AAAAGATAAC |
| 1380 | GACTGCGCCTCTAAAGATTTCCCTTGGAT | 1711 | CGTTTATAGTGTTTTAGGTGGTTGGCAC |
| | GAGCTACCGATTGACTTAATCCCCCAACA | | CCCTACCGACATAGCTATATCAACCCTC |
| | AAAGTCGTTTC | | AATAAATTTAT |
| 1381 | TCACACAATTGACCAACTATTAGTAACTC | 1712 | CTAATAATTGTATCAAATATGGAACGCA |
| | ACGCAGATACTGATCATATGGGGGATATC | | TACCGAAGTGTGAGTTCTGAAATTGATA |
| | GAAGTGGTTG | | CAATACAACT |
| 1382 | TCACACAATTGACCAACTATTAGTAACTC | 1713 | CTAATAATTGTATCAAATATGGAACGCA |
| | ACGCAGATACTGATCATATGGGGGATATC | | TACCGAAGTGTGAGTTCTGAAATTGATA |
| | GAAGTGGTTG | | CAATACAACT |
| 1383 | CCATCATAAGATGCCTTTTTACCGACGAG | 1714 | AAAGCATTATTTAGGCACTACAACTAGT |
| | TATAGTTGTACATGCCATTATCGGTCTCC | | ATAGTTGTACATGAAAAACGCTGTATTT |
| | TTTACAAACG | | TTTTATCCAT |
| 1384 | CCATCATAAGATGCCTTTTTACCGACGAG | 1715 | AAAGCATTATTTAGGCACTACAACTAGT |
| | TATAGTTGTACATGCCATTATCAGTCTCC | | ATAGTTGTACATGAAAAACGCTGTATTT |
| | TTTACAAACG | | TTTTATCCAT |
| 1385 | CCATCATAAGATGCCTTTTTACCGACGAG | 1716 | AAAGCATTATTTAGGCACTACAACTAGT |
| | TATAGTTGTACATGCCATTATCAGTCTCC | | ATAGTTGTACATGAAAAACGCTGTATTT |
| | TTTACAAACG | | TTTTATCCAT |
| 1386 | ACGTTTGTAAAGGAGACTGATAATGGCAT | 1717 | TGGATAAAAAAATACAGCGTTTTTCATG |
| | GTACAACTATACTCGTCGGTAAAAAGGCA | | TACAACTATACTCGTTGTAGTGCCTAAA |
| | TCTTATGATGG | | TAATGCTTTTA |
| 1387 | ACCTCCGCGCGGTCGCGCCGCGTGCGGTC | 1718 | AACGATGCTCGCGAGTCCTTTAGAGACA |
| | GTTCACCCAGGGGTCCGGCAGGAACAGCC | | CTGACCCACGTCAGTGGATCTAAAGGAC |
| | GCCAGTTGACG | | CACATCGGAGC |
| 1388 | ACAATCAACAAAGATGTATGGTGGTACAT | 1719 | TAACTTATGTACGGAAGTATAGACACTC |
| | GCATTAATATCGGATGTATACCTACTAAA | | GATTAATATTTAATGTGTATACTTCCGT |
| | ACATTAATTC | | AAAAATAACC |

Alternative Recognition Sites

| | | | |
|---|---|---|---|
| 1832 | AAAATATTTAGTTTTCTTTGGAGGAGCTG | 1888 | TTTTTAAATTTTGGTAATTAATGGAGTG |
| | GGACATCAACGGATAGCGGTGTTAAAGAT | | AACATCAACTGAAATTACTTCTATAAAC |
| | TTTCGGGGAA (rev comp*) | | TACCAAAATA (rev comp) |
| 1833 | AACAGTTCCTTTTTCAATGTTACTGTATC | 1889 | TTATTTATAGACTTTTTGTCAAATATAG |
| | CTGATGTGTACCTATAGCCCATCCGTCGC | | TGATGTGTACTTTACAAAAACACTATTT |
| | GCAATGAAAG | | TATATAAATA |
| 1834 | AACCAGCTGTAACTTTTTCGGTTCAAGCT | 1890 | TTAGCTTATTTAGTACCTCGTTTTCTCT |
| | ATGAGGGACGCAAAGAGGGAACTAAACAC | | CGTTGGAGGGAGAAGAAACGGGATACCA |
| | TTAATTGGTGT | | AAAATAAAGAC |
| 1835 | AAGTGTAATATGTTTGGGTATGGGGAAGT | 1891 | GAAAAAAAGTGTACATGGTAGAGAGTTA |
| | GAATCAGTACAATCGCCACAGTACACTTA | | AACCAGTTTAATACTCCACCATGTACAC |
| | TGTCAGCCTA (rev comp) | | GAAGTGAAAA (rev comp) |
| 1836 | AATGAGCTAAAAGCTGTGGCCCAGTCATC | 1892 | TTTATTTAATGTAGTTAGGTTGTGTTTA |
| | AATTGACCAAACCATGGTGTTTGAAATGC | | ATTGACCAAACACTATATAACTACAATA |
| | ACTGCCGCCA (rev comp) | | AAAGAGCACA (rev comp) |
| 1837 | ACAATCAACAAAGATGTATGGCGGTACAT | 1893 | TAACTTATGTACGGAAGTATAGACACTT |
| | GCATTAATATCGGATGTATACCGACTAAA | | GATTAATATTTAATGTGTATACTTCCGT |
| | ACATTAATTC (rev comp) | | ATTTTTATAG (rev comp) |
| 1838 | ACAATCGTCAGATAATTTTGGCGGTACAT | 1894 | TTAATAAACTATGGAAGTATGTACAGTC |
| | GCATAAATCACGGCTGTATCCCCTCTAAA | | TTGCAATGTTGAGTGAACAAACTTCCAT |
| | GTGCTCGTGC | | AATAAAATAA |
| 1839 | ACCAGCTGTAACTTTTTCGGATCAAGCTA | 1895 | TAGATTATTTAGTACCTCGTTATCTCTC |
| | TGAGGGACGCAAAGAGGGAACTAAACACT | | GCTGGACGGAGACGAATCGAGAAACTAA |
| | TAATTGGTGTT | | AATTATAAATA |
| 1840 | ACCGTAAAATAGCATTTCAGTTTTTCCAG | 1896 | GTTATCTTTTTATGTATTCATTTCGGGC |
| | CCCCGCACACAGCCCAAATAAAAAAAGAG | | TATTCAAGTAGCTGGTCTTGAATACCGA |
| | TCTTTCTTCT (rev comp) | | AAAAAATTCA (rev comp) |
| 1841 | AGCAACGCCAGATAGAACAGCATGATCTT | 1897 | AGCATGGTTTGTATATTGGCTAACGTTC |
| | CGGGTTGCCGAGCGTGACCAGCGTGCCGG | | GGGTTGCCGAGCGTTAGCCAATATACAT |
| | CCGCGAACATG (rev comp) | | ATTAACAGGGC (rev comp) |
| 1842 | AGCTTTCATTGCGCGACGGATGGGCTATA | 1898 | TATTTATATAAAATAGTGTTTTTGTAAA |
| | GGTACACATCAGGTTACAGTAACATTGAA | | GTACACATCACCATATTTGACAAAAAAC |
| | AAAGGAACTG | | CTATAAATAA |
| 1843 | ATAATCATCAAAGATTTTAGGATTATCAA | 1899 | TACTTTAATTTTAGGTTAATGGTCCATT |
| | ATTCACTATGATACGCCCTTCCGAAAGCT | | TCCTCTAGTAAATGTTTTATTAACCCAA |
| | GATACTAACGA (rev comp) | | AAAAAGAGTCT (rev comp) |
| 1844 | ATAATCATCAAAGATTTTCGGATTATCAA | 1900 | TACTTTAATTTTAGGTTAATGGTCCATT |
| | ATTCACTATGATATGCCCTGCTGAAAGCT | | TCCTCTAGTAAATGTTTAATTAACCCAA |
| | GATACTAACGA | | AAAAAGAGTCT |
| 1845 | ATCTTTTAACTGCAAAAGTACTACGGTCT | 1901 | CCACACGTGTAAGCAGTCCTACACACTC |
| | CTACATGAGCTGTTTGCGGGAACATATCG | | GATGTGCGTTGAGAGTACACTCTGTATC |
| | ACTGGTTGCA | | TTCCTACTAT |
| 1846 | ATCTTTTAACTGCAAAAGTACTACGGTCT | 1902 | CCACACGTGTAAGCAGTCCTACACACTC |
| | CTACATGAGCTGTTTGCGGGAACATATCG | | GATGTGCGTTGAGAGTACACTCTGTATC |
| | ACTGGTTGCA (rev comp) | | TTCCTACTAT (rev comp) |
| 1847 | ATGAATTAATGTTTTAGTAGGTATACATC | 1903 | TATAAAAAATACGGAAGTATACACATTA |
| | CGATATTAATGCATGTACCACCATACATC | | AATATTAATCAGGTGTCTATACTTCCGT |
| | TTTGTTGATT (rev comp) | | ACATACGTTA (rev comp) |
| 1848 | ATGTACGAGTACTTTAGACGGGATACAAC | 1904 | GTATAAATATATGGAAGTACACACATTA |
| | CGTGGTTAATGCACGTGCCGCCATAGTTA | | TACATTGCTCAATTGTGCATACTTCCAT |
| | TCTGATGATT | | ACTAAATTAA |
| 1849 | ATTTAACATCAATGAACCTGAACCCATGG | 1905 | CACGGCATTGTATTAAACTCAGTAAGAT |
| | TTGGATCAAAAACACTAAAGAATCGTCGT | | TATTTCTATGTTCCTACTGATTTTGATA |
| | TCTTTTTGAT (rev comp) | | CAAAAGAAAA (rev comp) |
| 1850 | ATTTAACATCAATGAACCTGAACCCATGG | 1906 | CACGGCATTGTATTAAACTCAGTAAGAT |
| | TTGGATCAAAAACACTAAAGAATCGTCGT | | TATTTCTATGTTCCTACTGATTTTGATA |
| | TCTTTTTGAT (rev comp) | | CAAAAGAAAA (rev comp) |
| 1851 | ATTTATTTCGTTCCGTGTTAGGTAATATT | 1907 | GTAGGCTCTTTTTGGGTTAATATAACAC |
| | ACGAGTAGCGAAGAAGGTCTGCCAAAAGA | | TCACTAGAGTCAATGTTCCTTTAACCCA |
| | AAATTTAGATT (rev comp) | | AAAATTAAAGG (rev comp) |
| 1852 | CACTCCCAAAGTCGGCTTCGTCAGTCTTG | 1908 | CCCCTAGTATAGGATGGGTTTCGTTAGG |
| | GATGCCCCAAGGCGCTGGTCGACTCCGAG | | GTGCCCCAACGAATAGAAAAGTAAACTA |
| | CGCATCCTCA | | GCTTTCAGCG |
| 1853 | CACTCCCAAAGTCGGCTTCGTCAGTCTTG | 1909 | CCCCTAGTATAGGATGGGTTTCGTTAGG |
| | GATGCCCCAAGGCGCTGGTCGACTCCGAG | | GTGCCCCAATGACTGCAAAAGTAAACTC |
| | CGCATCCTCA (rev comp) | | AATCTTTAAG (rev comp) |
| 1854 | CCATCATAAGATGCCTTTTTACCGACAAG | 1910 | AAAGCATTATTTAGGCACTACAACTAGT |
| | TATAGTTGTACATGCCATTATCAGTCTCC | | ATAGTTGTACATGAAAAACGCTGTATTT |
| | TTTACAAACG (rev comp) | | TTTTATCCAT (rev comp) |
| 1855 | CCATCATAAGATGCCTTTTTACCGACGAG | 1911 | AAAGCATTATTTAGGCACTACAACTAGT |
| | TATAGTTGTACATGCCATTATCGGTCTCC | | ATAGTTGTACATGAAAAACGCTGTATTT |
| | TTTACAAACG | | TTTTATCCAT |
| 1856 | CCATCATAAGATGCCTTTTTACCGACGAG | 1912 | AAAGCATTATTTAGGCACTACAACTAGT |
| | TATAGTTGTACATGCCATTATCAGTCTCC | | ATAGTTGTACATGAAAAACGCTGTATTT |
| | TTTACAAACG (rev comp) | | TTTTATCCAT (rev comp) |
| 1857 | CTGAGTGGGCGAACTATTTATCTTTTACA | 1913 | AATAATATTTTTATCCTTATTGACATAT |
| | ATGCCAAGCGGGTATAGCGGGAAGAAAGG | | GAGGAATCCCATGTATAATTAGGGGATA |
| | ACAAAATTTA (rev comp) | | AAAATAAAAA (rev comp) |
| 1858 | GAAACTATGGGGATTATAGCGTTTGAGGG | 1914 | GAATAGCTTTTTGCCATATTGACATACT |
| | AGCAAGTGCGGTTGGTAAGAGCACAACGT | | GCAAGTGCGGTGTATAATTAAGGCATAA |
| | GTCGTGAGTTA (rev comp) | | AATAAAAACTG (rev comp) |
| 1859 | GAAGGGAATAATAGCTCTGTTTTGCCTGC | 1915 | GTGGAATTTTTAGTATTCATAACGGGCT |
| | TCCACAAACTGCCCAAATCAAATATTCCG | | ATTCAAACAACCAATCATGAATACTAAA |
| | ACAGCCCTGGT | | ATTATCATAAA |
| 1860 | GACCACAATCCGCGTGTGGGCTTTGTATC | 1916 | GAAGCCGTATAGTATAGGAATGGTGTCG |
| | CCTTGGGTGCCCCAAGGCACTCGTCGATT | | CTTGGGTGCCCGTAGGATAGCAAAAGTA |
| | CGGAGCAGATC (rev comp) | | TACTCATCGCT (rev comp) |
| 1861 | GCGAACGCCACTGCGGCCCCATCAGCAGC | 1917 | TTACTGCGGTGTACATTATTGCATGACT |
| | AATGAACAGTCAGTCGTACCACCGCCGAT | | ACGAACAGTTATGTTATGATGTACACCA |
| | ATCCACCACCA (rev comp) | | CAGTTAATGGA (rev comp) |
| 1862 | GCGAACGCCACTGCGGTCCCATCAGCAGC | 1918 | TTACTGCGGTGTACATTCTTGCATGACT |
| | AATGAACAGTCAGTCGTACCACCGCCGAT | | ACGAACAGTTATGTTATGATGTACACCA |
| | ATCCACCACCA (rev comp) | | CAGTTAATGGA (rev comp) |
| 1863 | GCTGCCGATCACCGAGATCGCGTTCGCGT | 1919 | CTCTCCTGAAGTGTCAGTTGAGCGCCTT |
| | CCGGCTTCGCCAGCGTGCGGCAGTTCAAC | | CGGTTTTCCGAGTGCGCGTGAACTACAG |
| | GACACGATCC | | TTCTAGCATG |
| 1864 | GGAAATTAATGAGCCGTTTGACCACTGAT | 1920 | CAGGGTTACTTTATACAACATTAATCTG |
| | CTTTTTGAAATTTCGGAAGTGGCGCATCA | | TATTTGAAAATAAAGAGCAATGTTGTAC |
| | TGGTCCAGAAG | | ATCAAGATACA |
| 1865 | GGAAATTAATGAGCCGTTTGACCACTGAT | 1921 | TAGTAATATTATATGCAACATTATTCTG |
| | CTTTTTGAAATTTCGGAAGTGGCGCATCA | | TATTTGAAAATAAAGAGCAATGTTGTAC |
| | TGGTCCAGAAG (rev comp) | | ATCAAGATACA (rev comp) |
| 1866 | GGTGAGGATGCGCTCGGAGTCGACCAGCG | 1922 | CGCTGAAAGCTAGTTTACTTTTCTATTC |
| | CCTTGGGGCATCCAAGACTGACGAAGCCG | | GTTGGGGCACCCTAACGAAACCCATCCT |
| | ACTTTGGGAG | | ATACTAGGGG |
| 1867 | GGTGAGGATGCGCTCGGAGTCGACCAGCG | 1923 | CGCTGAAAGCTAGTTTACTTTTCTATTC |
| | CCTTGGGGCATCCAAGACTGACGAAGCCG | | GTTGGGGCACCCTAACGAAACCCATCCT |
| | ACTTTGGGAG (rev comp) | | ATACTAGGGG (rev comp) |
| 1868 | GTCTTCTGGACCATGATGCGCTACTTCCG | 1924 | TGTATCTTGATGTACAACATTGCTCTTT |
| | AAATTTCAAAAAGATCAGTGGTCAAACGG | | ATTTTCAAATACAGAATAATGTTGCATA |
| | CTCATTAATTT | | TAATATCACTA |
| 1869 | GTGGATCACCTGGTTTTTCGTGTTCAGAT | 1925 | CTCCTTTTATTAGGGTTTGTGTCATCTA |
| | ACAGGCATACGAAGTGCTCCTGAGACAGA | | CACACATGTAAAGTTTACATAAACCCTA |
| | AAGCGCATAT | | AAAAGATCGA |
| 1870 | TAACACCAATTAAATGTTTAGTTCCCTCT | 1926 | GTCTTTATTTTTGGTATCCCGTTTCTTC |
| | TTGCGTCCCTCATAGCTTGATCCGAAAAA | | TCCCTCCAACGAGAGAAAACGAGGAACT |
| | GTTACAGCTGG (rev comp) | | AAACAATCTAA (rev comp) |
| 1871 | TAACACCAATTAAGTGTTTAGTTCCCTCT | 1927 | GTCTTTATTTTTGGTATCCCGTTTCTTC |
| | TTGCGTCCCTCATAGCTTGAACCGAAAAA | | TCCCTCCAACGAGAGAAAACGAGGAACT |
| | GTTACAGCTGG | | AAACAATCTAA |
| 1872 | TAACACCAATTAAGTGTTTAGTTCCCTCT | 1928 | ATGTTCTTTTTTGGTATCTCGTTTATTC |
| | TTGCGTCCCTCATAGCTTGATCCGAAAAA | | TTCTTCCAACGAGAGGAAACGAGGAACT |
| | GTTACAGCTGG (rev comp) | | AAACAATCTAA (rev comp) |
| 1873 | TAACACCAATTAAGTGTTTAGTTCCCTCT | 1929 | TGTTCTTTTTTTGGTATCTCGTTTCTTC |
| | TTGCGTCCCTCATAGCTTGATCCGAAAAA | | TTCTTCCAACGAGAGGAAATGAGGCACT |
| | GTTACAGCTGG (rev comp) | | AAACCAGTTGA (rev comp) |
| 1874 | TACAAAGTAGATGTCTTTTGTAGCCATTA | 1930 | CGTTCGTGCTTTGTCGTCACCTTGTTGG |
| | GGCGCATTAGATTTACTCCATTAAGCCCC | | TGTAATTAGGTTGACGCCAACAGGGTGA |
| | AACGCATCAT (rev comp) | | TGACAATATA (rev comp) |
| 1875 | TACCCGTTGCTTCGTTGTAGCAACACTAC | 1931 | TTTCTAAGCTTTTACAAGCAGAGCAACA |
| | GCACTCCACGTGATGCGTATTTGGAAATA | | CACTCCACGTGTGGTGATAGGTCTTACC |
| | AATCAGCCGGC (rev comp) | | CATATTATGGA (rev comp) |
| 1876 | TACCCGTTGCTTCGTTGTAGCAACACTAC | 1932 | TTTCTAAGCTTTTACAAGCAGAGCAACA |
| | GCACTCCACGTGATGCGTATTTGGAAATA | | CACTCCACGTGTGGTGATAGGTCTTACC |
| | AATCAGCCGGC (rev comp) | | CATATTATGGA (rev comp) |
| 1877 | TATCTTTTAACTGCAAGAGTACTACAGTT | 1933 | TCTACACGAGTAAGCAGACCTACACACT |
| | TCCACGTGAGCTGTTTGCGGGAACATATC | | CGATGTGCATTGACTGTCTACTTAGTAT |
| | GACGGGTTGCA (rev comp) | | CTTCCTACTAT (rev comp) |
| 1878 | TATCTTTTAACTGCAAGAGTACTACGGTT | 1934 | TCTTGGCGAGTGAGCAGACCTATACACT |
| | TCCACGTGAGCTGTTTGCGGGAACATATC | | CGATGTGCGTTGACTGTCTACTTAGTAT |
| | GACGGGTTGCA (rev comp) | | CTTCCTACTAT (rev comp) |
| 1879 | TATCTTTTAACTGCAAGAGTACTACGGTT | 1935 | TCCACACGTGTAAGCAGTCCTACACACT |
| | TCCACGTGAGCTGTTTGCGGGAACATATC | | CGATGTGCGTTGAGAGTACACTCTGTAT |
| | GACGGGTTGCA (rev comp) | | CTTCCTACTAT (rev comp) |
| 1880 | TATGCAACCCGTCGATATGTTCCCGCAAA | 1936 | ATAGTAGGAAGATACTAAGTAGACAGTC |
| | CAGCTCACGTGGAAACCGTAGTACTCTTG | | AACGCACATCGAGTGTATAGGTCTGCTC |
| | CAGTTAAAAGA (rev comp) | | ACTCGCCAAGA (rev comp) |
| 1881 | TATGCAACCCGTCGATATGTTCCCGCAAA | 1937 | ATAGTAGGAAGATACTAAGTAGACAGTC |
| | CAGCTCACGTGGAAACCGTAGTACTCTTG | | AACGCACATCGAGTGTATAGGTCTGCTC |
| | CAGTTAAAAGA (rev comp) | | ACTCGCCAAGA (rev comp) |
| 1882 | TCCCTTAGGTGCTAATAGCGCCACTAATT | 1938 | CCACACGTGTAAGCAGTCCTACACACTC |
| | CCACATGATGTGTTTGTGGGAATAAATCG | | GATGTGCGTTGAGAGTACACTCTGTATC |
| | ACTGGTTGTA (rev comp) | | TTCCTACTAT (rev comp) |
| 1883 | TCCCTTAGGTGCTAATAGCGCCACTAATT | 1939 | CCACACGTGTAAGCAGTCCTACACACTC |
| | CCACATGATGTGTTTGTGGGAATAAATCG | | GATGTGCGTTGAGAGTACACTCTGTATC |
| | ACTGGTTGTA (rev comp) | | TTCCTACTAT (rev comp) |
| 1884 | TCGGGGCACGGTATTGGTGATTCACGAGA | 1940 | TATTAGTTAGATGTCATAGACCGATTTA |
| | ACAAGGGGCTCAACGACTGGGTTCGGTCC | | CAGCGGACTGTAGGTTGATCTAGGACAC |
| | GTCGCGGGAC (rev comp) | | CTAACCAATA (rev comp) |
| 1885 | TTATTCTCTAATAAGTTTAACTACAGTCT | 1941 | GTGCTTTAGTCAACAATACTACGCTCTC |
| | CACAATGTGGCGTATTTGGGAACATATCC | | AACGTGGCTCGGTTCTCCAATGACCAAC |
| | ATACACTTAA (rev comp) | | CTATTCAACA (rev comp) |
| 1886 | TTATTCTCTAATAAGTTTAACTACAGTCT | 1942 | GTGCTTTAGTCAACAATACTACGCTCTC |
| | CACAATGTGGCGTATTTGGGAACATATCC | | AACGTGGCTCGGTTCTCCAATGACCAAC |
| | ATACACTTAA (rev comp) | | CTATTCAACA (rev comp) |
| 1887 | TTTAAATTTTGTCCTTTCTTCCCGCTATA | 1943 | TTTTTATTTTTATCCCCTAATTATACAT |
| | CCCACTTGGCATTGTAAAAGATAAATAGT | | GGCATTCCTCATATGTCAATAAGGATAA |
| | TCGCCCACTC (rev comp) | | AAATATTATT (rev comp) |
| 1954 | TAACACCAATTAAATGTTTAGTTCCCTCT | 1959 | GTCTTTATTTTTGGTATCCCGTTTCTTC |
| | TTGCGTCCCTCATAGCTTGATCCGAAAAA | | TCCCTCCAACGAGAGAAATCGAGGTACT |
| | GTTACAGCTGG (rev comp) | | AAACAAGCTAA (rev comp) |
| 1955 | ACAATCATCAGATAACTATGGCGGCACGT | 1960 | TTAATTTAGTATGGAAGTATGCACAATT |
| | GCATTAACCACGGTTGTATCCCGTCTAAA | | GAGCAATGTATAATGTGTGTACTTCCAT |
| | GTACTCGTAC (rev comp) | | ATATTTATAC (rev comp) |
| 1956 | AATGTTTGTAAAGGAGACTGATAATGGCA | 1961 | ATGGATAAAAAAATACAGCGTTTTTCAT |
| | TGTACAACTATACTCGTCGGTAAAAAGGC | | GTACAACTATACTAGTTGTAGTGCCTAA |
| | ATCTTATGAT (rev comp) | | ATAATGCTTT (rev comp) |
| 1957 | GTCTTCTGGACCATGATGCGCCACTTCCG | 1962 | TGTATCTTGATGTACAACATTGCTCTTT |
| | AAATTTCAAAAAGATCAGTGGTCAAACGG | | ATTTTCAAATACAGATTAATGTTGTATA |
| | CTCATTAATTT (rev comp) | | AAGTAACCCTG (rev comp) |
| 1958 | TTTAAATTTTGTCCTTTCTTCCCGCTATA | 1963 | TTTTTATTTTTATCCCCTAATTATACAT |
| | CCCGCTTGGCATTGTAAAAGATAAATAGT | | GGCATTCCTCATATGTCAATAAGGATAA |
| | TCGCCCACTC (rev comp) | | AAATATTATT (rev comp) |

*revcomp:thereversecomplementsequencealignstothefirstdeclaredtargetsitemostclosely

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein.

What is claimed is:

1. A method comprising:
mining from a protein database putative recombinase sequences based on conserved recombinase domain architecture or other measure of homology to known recombinases;
linking the putative recombinase sequences to prokaryotic genomic sequences containing their corresponding coding sequences;
scanning those genomic sequences to identify prophage sequences containing the coding sequences;
aligning the prophage sequences and their boundary-flanking sequences with homologous genomic sequences, optionally, from the same genus to produce sequence alignments; and
automatically solving for putative cognate recombinase recognition sites by detecting overlapping sequences in the sequence alignments, thereby producing a solved recombinase list.

2. The method of claim 1, wherein the mining is based on a precisely ordered recombinase domain superfamily architecture or other measure of homology to known recombinases.

3. The method of claim 1, wherein the linking includes accessing a database that comprises annotated records of genomes assembled from long-read nucleotide sequences, short-read nucleotide sequences, or a combination of long- and short-read nucleotide sequences, or directly annotated records of long-read nucleotide sequences.

4. The method of claim 1, wherein the linking includes automatically removing uninformative nucleotide sequences from the genomic coding sequences.

5. The method of claim 1, wherein the genomic coding sequences includes at least 2, at least 5, at least 10, at least 25, at least 50, or at least 100 annotated genomic coding sequences.

6. The method of claim 1, wherein the boundary-flanking sequences have a length of at least 20 kilobases.

7. The method of claim 1, wherein the automatically solving includes defining multiple putative cognate recombinase recognition sites for a single recombinase.

8. The method of claim 1, wherein the automatically solving includes implementation of an algorithm that includes a measure of confidence in each predicted recombinase recognition site set, optionally in the form of ambiguity scores.

9. The method of claim 1, further comprising verifying that all putative cognate recombinase recognition sites solved flank a sequence encoding at least one of the putative recombinase sequences.

10. The method of claim 1, wherein the putative recombinase sequences comprise tyrosine and/or serine recombinase sequences, optionally wherein the serine recombinase sequences comprise resolvase and/or integrase sequences.

11. The method of claim 1, further comprising continuously updating the solved recombinase list as the protein database is updated.

12. A computer readable medium on which is stored a computer program which, when implemented by a computer processor, causes the processor to:
mine from a protein database putative recombinase sequences based on conserved recombinase domain architecture or other measure of homology to known recombinases;
link the putative recombinase sequences to prokaryotic genomic sequences containing their corresponding coding sequences;
scan those genomic sequences to identify prophage sequences containing the coding sequences;
align the prophage sequences and their boundary-flanking sequences with homologous genomic sequences from the same genus to produce sequence alignments; and
solve for putative cognate recombinase recognition sites by detecting overlapping sequences in the sequence alignments.

13. The computer readable medium of claim 12, wherein the mining is based on a precisely ordered recombinase domain superfamily architecture or other measure of homology to known recombinases.

14. The computer readable medium of claim 12, wherein the linking includes accessing a database that comprises annotated records of genomes assembled from long-read nucleotide sequences, short-read nucleotide sequences, or a combination of long- and short-read nucleotide sequences, or directly annotated records of long-read nucleotide sequences.

15. The computer readable medium of claim 12, wherein the linking includes automatically removing uninformative nucleotide sequences from the genomic coding sequences.

16. The computer readable medium of claim 12, wherein the solving includes (i) defining multiple putative cognate recombinase recognition sites for a single recombinase; or (ii) implementation of an algorithm that includes a measure of confidence in each predicted recombinase recognition site set, optionally in the form of ambiguity scores.

17. The computer readable medium of claim 12, further comprising verifying that all putative cognate recombinase recognition sites solved flank a sequence encoding at least one of the putative recombinase sequences.

18. The computer readable medium of claim 12, further comprising continuously updating the solved recombinase list as the protein database is updated.

19. A system configured to perform:
mining a protein database putative recombinase sequences based on conserved recombinase domain architecture or other measure of homology to known recombinases;
linking the putative recombinase sequences to prokaryotic genomic sequences containing their corresponding coding sequences;
scanning those genomic sequences to identify prophage sequences containing the coding sequences;
aligning the prophage sequences and their boundary-flanking sequences with homologous genomic sequences from the same genus to produce sequence alignments; and
solving for putative cognate recombinase recognition sites by detecting overlapping sequences in the sequence alignments.

20. The system of claim 19, wherein the system is a computer system.
